SEQUENCE LISTING (812 sequences)

SEQ ID NO: 1-2 - skipped (2 entries)

SEQ ID NO: 3 - RNA, 45 nt, Unknown
  Description of Unknown: GluR2 sequence
guggaauaguauaacaauaugcuaaauguuguuauaguaucccac

SEQ ID NO: 4 - RNA, 145 nt, Artificial Sequence
  Description of Artificial Sequence: Synthetic polynucleotide
auggguauggccucuaaaaacauggccccagcagcuucagucccuuucucgucgaugguccacccuaugauauuguuguaaaucguauaacaauaugauaaggugagcacagccuuaugcacggccuuggagagcuucaggggug

SEQ ID NO: 5 - RNA, 145 nt, Artificial Sequence
  Description of Artificial Sequence: Synthetic polynucleotide
auggguauggccucuaaaaacauggccccagcagcuucagucccuuucucgucgaugguccacccuaugauauuguuguaaaucguauaacaauaugauaaggugagcacagccuuaugcacggccuggaggggagagaagcaga

SEQ ID NO: 6 - RNA, 100 nt, Artificial Sequence
  Description of Artificial Sequence: Synthetic polynucleotide
auggguauggccucuaaaaacauggccccagcagcuucagucccuuucucgucgauggucagcacagccuuaugcacggccuuggagagcuucaggggug

SEQ ID NO: 7 - DNA, 106 bp, Artificial Sequence
  Description of Artificial Sequence: Synthetic polynucleotide
atgggtatggcctctaaaaacatggccccagcagcttcagtcatacctttctcgtcgatggtcagcatgacagccttatgcacggccttggagagcttcaggggtg

SEQ ID NO: 8 - RNA, 100 nt, Artificial Sequence
  Description of Artificial Sequence: Synthetic polynucleotide
auggguauggccucuaaaaacauggccccagcagcuucagucccuuucucgucgauggucagcacagccuuaugcacggccuggaggggagagaagcaga

SEQ ID NO: 9 - RNA, 100 nt, Artificial Sequence
  Description of Artificial Sequence: Synthetic polynucleotide
auggguauggccucuaaaaacauggccccagcagcuucagucccggucucgucgauggucagcacagccguaugcacggccuggaggggagagaagcaga

SEQ ID NO: 10 - RNA, 106 nt, Artificial Sequence
  Description of Artificial Sequence: Synthetic polynucleotide
auggguauggccucuaaaaacauggccccagcagcuucagucauaccuuucucgucgauggucagcaugacagccuuaugcacggccuggaggggagagaagcaga

SEQ ID NO: 11 - RNA, 100 nt, Artificial Sequence
  Description of Artificial Sequence: Synthetic polynucleotide
ccccagugagcaucuugaaugugguuguauugccggcaccauucacucccaggaggccaaagcacucuccagugagaacucggaccacagccucccgcug

SEQ ID NO: 12 - RNA, 100 nt, Artificial Sequence
  Description of Artificial Sequence: Synthetic polynucleotide
ccccagugagcaucuugaaugugguuguauugccggcaccauucacucccaggaggccaaagcacucuccagugagaacucggacaccagccucccgcug

SEQ ID NO: 13 - RNA, 100 nt, Artificial Sequence
  Description of Artificial Sequence: Synthetic polynucleotide
ccccagugagcaucuugaaugugguuguauugccggcaccauucacucccaggaggccaaagcacucuccagggagaacucggaccacagccucccacug

SEQ ID NO: 14 - RNA, 100 nt, Artificial Sequence
  Description of Artificial Sequence: Synthetic polynucleotide
ccccagugagcaucuugaaugugguuguauugccggcaccauucacucccaggaggccaaagcacucuccagggagaacucggacaccagccucccacug

SEQ ID NO: 15 - RNA, 100 nt, Artificial Sequence
  Description of Artificial Sequence: Synthetic polynucleotide
cccagugagcaucuugaaugugguuguauugccggcaccauucacucccaggaggccaaagcacucuccagugcgaacucggaccacagccuguccgcug

SEQ ID NO: 16 - RNA, 100 nt, Artificial Sequence
  Description of Artificial Sequence: Synthetic polynucleotide
cccagugagcaucuugaaugugguuguauugccggcaccauucacucccaggaggccaaagcacucuccagugcgaacucggacaccagccuguccgcug

SEQ ID NO: 17 - skipped

SEQ ID NO: 18 - DNA, 150 bp, Artificial Sequence
  Description of Artificial Sequence: Synthetic polynucleotide
tactcttgcctgctacggtggcatcccctgaggtcactgtggtgtccccagtgagcatcttgaatgtggttgtattgccggcaccattcactcccaggaggccaaagcactctccagggcgaactccgacacacagcctgtccactgctg

SEQ ID NO: 19 - DNA, 150 bp, Artificial Sequence
  Description of Artificial Sequence: Synthetic polynucleotide
aatactcttgcctgctacggtggcatcccctgaggtcactgtggtgtccccagtgagcatcttgaatgtggttgtattgccggcaccattcactcccaggaggccaaagcactctccagggcgaactcacacacagcctgtccactgctg

SEQ ID NO: 20 - DNA, 150 bp, Artificial Sequence
  Description of Artificial Sequence: Synthetic polynucleotide
atactcttgcctgctacggtggcatcccctgaggtcactgtggtgtccccagtgagcatcttgaatgtggttgtattgccggcaccattcactcccaggaggccaaagcactctccagggcgaactcgacacacagcctgtccactgctg

SEQ ID NO: 21 - DNA, 150 bp, Artificial Sequence
  Description of Artificial Sequence: Synthetic polynucleotide
aatactcttgcctgctacggtggcatcccctgaggtcactgtggtgtccccagtgagcatcttgaatgtggttgtattgccggcaccattcactcccaggaggccaaagcactctccagtgagaactcggaccacagcctcccgctgctg

SEQ ID NO: 22 - DNA, 150 bp, Artificial Sequence
  Description of Artificial Sequence: Synthetic polynucleotide
aatactcttgcctgctacggtggcatcccctgaggtcactgtggtgtccccagtgagcatcttgaatgtggttgtattgccggcaccattcactcccaggaggccaaagcactctccagtgagaactcggacaccagcctcccactgctg

SEQ ID NO: 23 - DNA, 150 bp, Artificial Sequence
  Description of Artificial Sequence: Synthetic polynucleotide
aatactcttgcctgctacggtggcatcccctgaggtcactgtggtgtccccagtgagcatcttgaatgtggttgtattgccggcaccattcactcccaggaggccaaagcactctccagggagaactcggaccacagcctcccactgctg

SEQ ID NO: 24 - DNA, 150 bp, Artificial Sequence
  Description of Artificial Sequence: Synthetic polynucleotide
aatactcttgcctgctacggtggcatcccctgaggtcactgtggtgtccccagtgagcatcttgaatgtggttgtattgccggcaccattcactcccaggaggccaaagcactctccagggagaactcggacaccagcctcccactgctg

SEQ ID NO: 25 - DNA, 150 bp, Artificial Sequence
  Description of Artificial Sequence: Synthetic polynucleotide
gtgagcatcttgaatgtggttgtattgccggcaccattcactcccaggaggccaaagcactctccagggcgaactccgacacacagcctgtccactgctgggctggaggtgcctggataaatcttggttagttcatgtagccttaagatg

SEQ ID NO: 26 - DNA, 150 bp, Artificial Sequence
  Description of Artificial Sequence: Synthetic polynucleotide
gtgagcatcttgaatgtggttgtattgccggcaccattcactcccaggaggccaaagcactctccagggcgaactcggacacacagcctgtccactgctgggctggaggtgcctggataaatcttggttagttcatgtagccttaagatg

SEQ ID NO: 27 - DNA, 150 bp, Artificial Sequence
  Description of Artificial Sequence: Synthetic polynucleotide
cagtgagcatcttgaatgtggttgtattgccggcaccattcactcccaggaggccaaagcactctccagggcgaactcacacacagcctgtccactgctgggctggaggtgcctggataaatcttggttagttcatgtagccttaagatg

SEQ ID NO: 28 - DNA, 150 bp, Artificial Sequence
  Description of Artificial Sequence: Synthetic polynucleotide
agtgagcatcttgaatgtggttgtattgccggcaccattcactcccaggaggccaaagcactctccagggcgaactcgacacacagcctgtccactgctgggctggaggtgcctggataaatcttggttagttcatgtagccttaagatg

SEQ ID NO: 29 - DNA, 150 bp, Artificial Sequence
  Description of Artificial Sequence: Synthetic polynucleotide
cagtgagcatcttgaatgtggttgtattgccggcaccattcactcccaggaggccaaagcactctccagtgagaactcggaccacagcctcccgctgctgggctggaggtgcctggataaatcttggttagttcatgtagccttaagatg

SEQ ID NO: 30 - DNA, 150 bp, Artificial Sequence
  Description of Artificial Sequence: Synthetic polynucleotide
cagtgagcatcttgaatgtggttgtattgccggcaccattcactcccaggaggccaaagcactctccagtgagaactcggacaccagcctcccactgctgggctggaggtgcctggataaatcttggttagttcatgtagccttaagatg

SEQ ID NO: 31 - DNA, 150 bp, Artificial Sequence
  Description of Artificial Sequence: Synthetic polynucleotide
cagtgagcatcttgaatgtggttgtattgccggcaccattcactcccaggaggccaaagcactctccagggagaactcggaccacagcctcccactgctgggctggaggtgcctggataaatcttggttagttcatgtagccttaagatg

SEQ ID NO: 32 - DNA, 150 bp, Artificial Sequence
  Description of Artificial Sequence: Synthetic polynucleotide
cagtgagcatcttgaatgtggttgtattgccggcaccattcactcccaggaggccaaagcactctccagggagaactcggacaccagcctcccactgctgggctggaggtgcctggataaatcttggttagttcatgtagccttaagatg

SEQ ID NO: 33 - DNA, 150 bp, Artificial Sequence
  Description of Artificial Sequence: Synthetic polynucleotide
agtgagcatcttgaatgtggttgtattgccggcaccattcactcccaggaggccaaagcactctccagtgcgaactcggaccacagcctgtccgctgctgggctggaggtgcctggataaatcttggttagttcatgtagccttaagatg

SEQ ID NO: 34 - DNA, 150 bp, Artificial Sequence
  Description of Artificial Sequence: Synthetic polynucleotide
agtgagcatcttgaatgtggttgtattgccggcaccattcactcccaggaggccaaagcactctccagtgcgaactcggacaccagcctgtccgctgctgggctggaggtgcctggataaatcttggttagttcatgtagccttaagatg

SEQ ID NO: 35 - DNA, 124 bp, Artificial Sequence
  Description of Artificial Sequence: Synthetic polynucleotide
atatgctaatacgactcactataggtgccctctgatgtttttatccccattctacagcagtactgagcaatgccgtagtcagcaatctttgcaatgatggcagcattgggatacagtgtgaaaa

SEQ ID NO: 36 - DNA, 169 bp, Artificial Sequence
  Description of Artificial Sequence: Synthetic polynucleotide
atatgctaatacgactcactataggtgccctctgatgtttttatccccattctacagcagtactgagcaatgccgtagtcagcaatctttgcaatgatggcagcattgggatacagtgtgaaaagtggaatagtataacaatatgctaaatgttgttatagtatcccac

SEQ ID NO: 37 - DNA, 214 bp, Artificial Sequence
  Description of Artificial Sequence: Synthetic polynucleotide
atatgctaatacgactcactataggtggaatagtataacaatatgctaaatgttgttatagtatcccacgtgccctctgatgtttttatccccattctacagcagtactgagcaatgccgtagtcagcaatctttgcaatgatggcagcattgggatacagtgtgaaaagtggaatagtataacaatatgctaaatgttgttatagtatcccac

SEQ ID NO: 38 - skipped

SEQ ID NO: 39 - DNA, 111 bp, Artificial Sequence
  Description of Artificial Sequence: Synthetic polynucleotide
atatgctaatacgactcactatagaaggacgggtcgtactgagcaatgccctagtcaaagtggacagcaatctttgcaacgatggcagcatcgggatacacctgtgactaa

SEQ ID NO: 40 - DNA, 169 bp, Artificial Sequence
  Description of Artificial Sequence: Synthetic polynucleotide
atatgctaatacgactcactataggtgccctctgatgttcttatccccattccacagcagtactgagcaatgccgtagtcacaccctatgatattgttgtaaatcgtataacaatatgataaggtggcaatcttcgcaatgatggcagcattgggacacagtgtgaaaa

SEQ ID NO: 41 - DNA, 169 bp, Artificial Sequence
  Description of Artificial Sequence: Synthetic polynucleotide
atatgctaatacgactcactataggtgccctctgatgttcttatccccattccacagcagtactgagcaatgccgtagtcagtggaatagtataacaatatgctaaatgttgttatagtatcccacgcaatcttcgcaatgatggcagcattgggatacagtgtgaaaa

SEQ ID NO: 42 - DNA, 111 bp, Artificial Sequence
  Description of Artificial Sequence: Synthetic polynucleotide
atatgctaatacgactcactatagaaggacgggtcgtactgagcaatgccgtagtcaaagtggacagcaatctttgcaacgatggcagcatcgggatacacctgtgactaa

SEQ ID NO: 43 - DNA, 21 bp, Artificial Sequence
  Description of Artificial Sequence: Synthetic oligonucleotide
ttttcacactgtatcccaatg

SEQ ID NO: 44 - DNA, 19 bp, Artificial Sequence
  Description of Artificial Sequence: Synthetic oligonucleotide
ttagtcacaggtgtatccc

SEQ ID NO: 45 - skipped

SEQ ID NO: 46 - RNA, 100 nt, Artificial Sequence
  Description of Artificial Sequence: Synthetic polynucleotide
gugcccucugauguuuuuauccccauucuacagcaguacugagcaaugccguagucagcaaucuuugcaaugauggcagcauugggauacagugugaaaa

SEQ ID NO: 47 - RNA, 145 nt, Artificial Sequence
  Description of Artificial Sequence: Synthetic polynucleotide
gugcccucugauguuuuuauccccauucuacagcaguacugagcaaugccguagucagcaaucuuugcaaugauggcagcauugggauacagugugaaaaguggaauaguauaacaauaugcuaaauguuguuauaguaucccac

SEQ ID NO: 48 - RNA, 190 nt, Artificial Sequence
  Description of Artificial Sequence: Synthetic polynucleotide
guggaauaguauaacaauaugcuaaauguuguuauaguaucccacgugcccucugauguuuuuauccccauucuacagcaguacugagcaaugccguagucagcaaucuuugcaaugauggcagcauugggauacagugugaaaaguggaauaguauaacaauaugcuaaauguuguuauaguaucccac

SEQ ID NO: 49 - RNA, 100 nt, Artificial Sequence
  Description of Artificial Sequence: Synthetic polynucleotide
gugcccucugauguuuuuauccccauucgguuacaguaaugagcaaaugccgagucagcaagauuugcugguugggcagcauugggauacagugugaaaa

SEQ ID NO: 50 - RNA, 87 nt, Artificial Sequence
  Description of Artificial Sequence: Synthetic oligonucleotide
aaggacgggucguacugagcaaugcccuagucaaaguggacagcaaucuuugcaacgauggcagcaucgggauacaccugugacuaa

SEQ ID NO: 51 - RNA, 145 nt, Artificial Sequence
  Description of Artificial Sequence: Synthetic polynucleotide
gugcccucugauguucuuauccccauuccacagcaguacugagcaaugccguagucacacccuaugauauuguuguaaaucguauaacaauaugauaagguggcaaucuucgcaaugauggcagcauugggacacagugugaaaa

SEQ ID NO: 52 - RNA, 145 nt, Artificial Sequence
  Description of Artificial Sequence: Synthetic polynucleotide
gugcccucugauguucuuauccccauuccacagcaguacugagcaaugccguagucaguggaauaguauaacaauaugcuaaauguuguuauaguaucccacgcaaucuucgcaaugauggcagcauugggauacagugugaaaa

SEQ ID NO: 53 - RNA, 100 nt, Unknown
  Description of Unknown: target sequence
uuuucacacuguaucccaaugcugccaucauugcaaagauugcugacuacagcauugcucaguacugcuguagaauggggauaaaaacaucagagggcac

SEQ ID NO: 54 - RNA, 56 nt, Unknown
  Description of Unknown: target sequence
uguaucccaaugcugccaucauugcaaagauugcugacuacagcauugcucaguac

SEQ ID NO: 55 - DNA, 24 bp, Artificial Sequence
  Description of Artificial Sequence: Synthetic oligonucleotide
cgtagctgatggtttgagatacct

SEQ ID NO: 56 - DNA, 27 bp, Artificial Sequence
  Description of Artificial Sequence: Synthetic oligonucleotide
accaaatgaataaacatcagcctgttg

SEQ ID NO: 57 - DNA, 22 bp, Artificial Sequence
  Description of Artificial Sequence: Synthetic oligonucleotide
tttcctctggcaacttcaggtg

SEQ ID NO: 58 - DNA, 150 bp, Artificial Sequence
  Description of Artificial Sequence: Synthetic polynucleotide
tactcttgcctgctacggtggcatcccctgaggtcactgtggtgtccccagtgagcatcttgaatgtggttgtattgccggcaccattcactcccaggaggccaaagcactctccagggcgaactcggacacacagcctgtccactgctg

SEQ ID NO: 59 - DNA, 169 bp, Artificial Sequence
  Description of Artificial Sequence: Synthetic polynucleotide
taatagggatagggatagggacaactccctccttggcctttgaaagtcctttcatgaatacatccacggctaatgaattcctttacaccacactgtcgtcgaatggccactcccagttctcatataatttttggagcaggttttctgacttcggtcggaaaacccctcc

SEQ ID NO: 60 - DNA, 184 bp, Artificial Sequence
  Description of Artificial Sequence: Synthetic polynucleotide
taatagggatagggatagggacaactccctccttggcctttgaaagtcctttcatgaatacatccacggctaatgtggaatagtataacaatatgctaaatgttgttatagtatcccacgaattcctttacaccacatataatttttggagcaggttttctgacttcggtcggaaaacccctcc

SEQ ID NO: 61 - DNA, 197 bp, Artificial Sequence
  Description of Artificial Sequence: Synthetic polynucleotide
taatcaactccctccttggcctttgaaagtcctttcatgaatacaccctatgatattgttgtaaatcgtataacaatatgataaggtgcatccacggctaatgaattcctttacaccacactgtcgtcgaatggccactcccagttctcatataatttttggagcaggttttctgacttcggtcggaaaacccctcc

SEQ ID NO: 62 - DNA, 169 bp, Artificial Sequence
  Description of Artificial Sequence: Synthetic polynucleotide
taatagggatagggatagggacaactccctccttggcctttgaaagtcctttcatgaatacatccatagctaatgaattcctttacaccacactgtcgtcgaatggccactcccagttctcatataatttttggagcaggttttctgacttcggtcggaaaacccctcc

SEQ ID NO: 63 - DNA, 167 bp, Artificial Sequence
  Description of Artificial Sequence: Synthetic polynucleotide
taatagggatagggatagggaccctgtttggttctctcagcagcagccacaactccctccttggcctttgaaagtcctttcatgaatacatccatagctaatgaattcctttacaccacatataatttttggagcaggttttctgacttcggtcggaaaacccctcc

SEQ ID NO: 64 - DNA, 151 bp, Artificial Sequence
  Description of Artificial Sequence: Synthetic polynucleotide
taatgccacaactccctccttggcctttgaaagtcctttgatgcatacatccacggctaatgaattcctttacaccacactggaaaacataaaatacactttgatataatttttggagcaggttttctgacttcggtcggaaaacccctcc

SEQ ID NO: 65 - DNA, 201 bp, Artificial Sequence
  Description of Artificial Sequence: Synthetic polynucleotide
taatgccacaactccctccttggcctttgaaagtcctttgatgcatacatccacggctaatgaattcctttacaggagagtcaattatgatattatagagtttgactgaaaaatcatgagaaaccctccaaagtgtgagactcttttaagtgtatataatttttggagcaggttttctgacttcggtcggaaaacccctcc

SEQ ID NO: 66 - DNA, 182 bp, Artificial Sequence
  Description of Artificial Sequence: Synthetic polynucleotide
taatgccacaactccctccttggcctttgaaagtcctttgatgcatacatccacggctaatgaattcctttacaccacattagccagaaggcttgaaggcaaggcgtgagggagcgcccaggacgctctcggagatataatttttggagcaggttttctgacttcggtcggaaaacccctcc

SEQ ID NO: 67 - DNA, 162 bp, Artificial Sequence
  Description of Artificial Sequence: Synthetic polynucleotide
taatgcttctgccacaccctgtttggttttctcaaagggtatgaagaaggggagcttggcctttgaaagtcctttgatgcatacatccacggctaatgaattcctttacaccacatataatttttggagcaggttttctgacttcggtcggaaaacccctcc

SEQ ID NO: 68 - DNA, 151 bp, Artificial Sequence
  Description of Artificial Sequence: Synthetic polynucleotide
taatgccacaactccctccttggcctttgaaagtcctttgatgcatacatccacggctatacttttcctttacaccacactggaaaacataaaatacactttgatataatttttggagcaggttttctgacttcggtcggaaaacccctcc

SEQ ID NO: 69 - DNA, 151 bp, Artificial Sequence
  Description of Artificial Sequence: Synthetic polynucleotide
taatgccacaactccctccttggcctttgaaagtcctttgatgcatatatttacggctaatgaattcctttacaccacactggaaaacataaaatacactttgatataatttttggagcaggttttctgacttcggtcggaaaacccctcc

SEQ ID NO: 70 - DNA, 151 bp, Artificial Sequence
  Description of Artificial Sequence: Synthetic polynucleotide
taatgccacaactccctccttggcctttgaaagtgctttcacgaatacatccacggctaatgaattcctttacaccacactggaaaacataaaatacactttgatataatttttggagcaggttttctgacttcggtcggaaaacccctcc

SEQ ID NO: 71 - DNA, 151 bp, Artificial Sequence
  Description of Artificial Sequence: Synthetic polynucleotide
taatgccacaactccctccttggcctttgaaagtgctttcacgaatacatccacggctatacttttcctttacaccacactggaaaacataaaatacactttgatataatttttggagcaggttttctgacttcggtcggaaaacccctcc

SEQ ID NO: 72 - DNA, 155 bp, Artificial Sequence
  Description of Artificial Sequence: Synthetic polynucleotide
taatcaactccctccttggcctttgaaagtcctttcatgaatacatccacggctaatgaattcctttacaccacactgtcgtcgaatggccactcccagttctcatatataaatttttggagcaggttttctgacttcggtcggaaaacccctcc

SEQ ID NO: 73 - DNA, 155 bp, Artificial Sequence
  Description of Artificial Sequence: Synthetic polynucleotide
taatcaactccctccttggcctttgaaagtcctttcatgaatacatccatggctaatgaattcctttacaccacactgtcgtcgaatggccactcccagttctcatatataaatttttggagcaggttttctgacttcggtcggaaaacccctcc

SEQ ID NO: 74 - DNA, 185 bp, Artificial Sequence
  Description of Artificial Sequence: Synthetic polynucleotide
taatgccacaactccctccttggcctttgaaagtcctttcatgaatacatccacggctaatgaattcctttacaccacattagccagaaggcttgaaggcaaggcgtgagggagcgcccaggacgctctcggagatatataaatttttggagcaggttttctgacttcggtcggaaaacccctcc

SEQ ID NO: 75 - DNA, 175 bp, Artificial Sequence
  Description of Artificial Sequence: Synthetic polynucleotide
taatcctccttggcctttgaaagtcctttcatgaatacatccacggctaatgaattcctttacaccacattagccagaaggcttgaaggcaaggcgtgagggagcgcccaggacgctctcggagatatataaatttttggagcaggttttctgacttcggtcggaaaacccctcc

SEQ ID NO: 76 - DNA, 165 bp, Artificial Sequence
  Description of Artificial Sequence: Synthetic polynucleotide
taatctttgaaagtcctttcatgaatacatccacggctaatgaattcctttacaccacattagccagaaggcttgaaggcaaggcgtgagggagcgcccaggacgctctcggagatatataaatttttggagcaggttttctgacttcggtcggaaaacccctcc

SEQ ID NO: 77 - DNA, 185 bp, Artificial Sequence
  Description of Artificial Sequence: Synthetic polynucleotide
taatgccacaactccctccttggcctttgaaagtcctttgatgcatacatccacggctaatgaattcctttacaccacattagccagaaggcttgaaggcaaggcgacagggagcgcccaggacgctctcggagatatataaatttttggagcaggttttctgacttcggtcggaaaacccctcc

SEQ ID NO: 78 - DNA, 198 bp, Artificial Sequence
  Description of Artificial Sequence: Synthetic polynucleotide
taatgccacaactccctccttggcctttgaaagtcctttgatgcatacatccacggctaatgaattcctttacaccacattagcaaaggcagaaggcttgaaggcaaggacaccgggagcgcccaggacgctctcggaggggccgggatatataaatttttggagcaggttttctgacttcggtcggaaaacccctcc

SEQ ID NO: 79 - DNA, 184 bp, Artificial Sequence
  Description of Artificial Sequence: Synthetic polynucleotide
taatgccacaactccctccttggcctttgaaagtcctttcacgaatacatccacggctaatgaattcctttacaccacattagccagaaggcttgaaggcaaggcggagggagcgcccaggacgctctcggagatatataaatttttggagcaggttttctgacttcggtcggaaaacccctcc

SEQ ID NO: 80 - DNA, 183 bp, Artificial Sequence
  Description of Artificial Sequence: Synthetic polynucleotide
taatgccacaactccctccttggcctttgaaagtcctttcacgaatacaccacggctaatgaattcctttacaccacattagccagaaggcttgaaggcaaggcggagggagcgcccaggacgctctcggagatatataaatttttggagcaggttttctgacttcggtcggaaaacccctcc

SEQ ID NO: 81 - DNA, 190 bp, Artificial Sequence
  Description of Artificial Sequence: Synthetic polynucleotide
taatctttgaaagtcctttcatgaatacatccacggctaatgaattcctttacaccacaaggggcgaatggccactcccagttctccgctcacgagggtggaaataattaaggcgtgagggagcgcccaggacgctctcatatataaatttttggagcaggttttctgacttcggtcggaaaacccctcc

SEQ ID NO: 82 - DNA, 185 bp, Artificial Sequence
  Description of Artificial Sequence: Synthetic polynucleotide
taatgccacaactccctccttggcctttcgccaaaatttcatgaatacatccacggctaatgaattcctttacaccacattagccagaaggcttgaaggcaaggcgtgagggagcgcccaggacgctctcggagatatataaatttttggagcaggttttctgacttcggtcggaaaacccctcc

SEQ ID NO: 83 - DNA, 130 bp, Artificial Sequence
  Description of Artificial Sequence: Synthetic polynucleotide
taatgccacaactccctccttggcctttgaaagtcctttgatgcatacatccacggctaatgaattcctttacaccacaatatataaatttttggagcaggttttctgacttcggtcggaaaacccctcc

SEQ ID NO: 84 - DNA, 155 bp, Artificial Sequence
  Description of Artificial Sequence: Synthetic polynucleotide
taatgccacaactccctccttggcctttgaaagtcctttcatgccctgatccacggctaatgacttcatttacaccacactgtcgtcgaatggccactcccagtatatataaatttttggagcaggttttctgacttcggtcggaaaacccctcc

SEQ ID NO: 85 - DNA, 155 bp, Artificial Sequence
  Description of Artificial Sequence: Synthetic polynucleotide
taatgccacaactccctccttggcctttgaaagtcctttcacgaatacatccacggctaatgaattcctttacaccacactgtcgtcgaatggccactcccagtatatataaatttttggagcaggttttctgacttcggtcggaaaacccctcc

SEQ ID NO: 86 - DNA, 155 bp, Artificial Sequence
  Description of Artificial Sequence: Synthetic polynucleotide
taatgccacaactccctccttggcctttgaaagtcctttcacgaatacatccacggctaatgaattcctttacaccacactggaaaacataaaatacactttgaatatataaatttttggagcaggttttctgacttcggtcggaaaacccctcc

SEQ ID NO: 87 - DNA, 155 bp, Artificial Sequence
  Description of Artificial Sequence: Synthetic polynucleotide
taatgccacaactccctccttggcctttgaataacggttcacgaatacatccacggctaatgaattcctttacaccacactggaaaacataaaatacactttgaatatataaatttttggagcaggttttctgacttcggtcggaaaacccctcc

SEQ ID NO: 88 - DNA, 180 bp, Artificial Sequence
  Description of Artificial Sequence: Synthetic polynucleotide
taatgccacaactccctccttggcctttgaataacggttcacgaatacatccacggctaatgaattcctttacaccacactggaaaacataaaatacactttgactgtcgtcgaatggccactcccagtatatataaatttttggagcaggttttctgacttcggtcggaaaacccctcc

SEQ ID NO: 89 - DNA, 210 bp, Artificial Sequence
  Description of Artificial Sequence: Synthetic polynucleotide
taatgccacaactccctccttggcctttgaataacggttcacgaatacatccacggctaatgaattcctttacaccacactggaaaacataaaatacactttgattagccagaaggcttgaaggcaaggcgtgagggagcgcccaggacgctctcggagatatataaatttttggagcaggttttctgacttcggtcggaaaacccctcc

SEQ ID NO: 90 - DNA, 155 bp, Artificial Sequence
  Description of Artificial Sequence: Synthetic polynucleotide
taatgccacaactccctccttggcctttgaaagtcctttcatgccctgatccacggctaatgaatccctttaaaccacactggaaaacataaaatacactttgaatatataaatttttggagcaggttttctgacttcggtcggaaaacccctcc

SEQ ID NO: 91 - skipped

SEQ ID NO: 92 - DNA, 168 bp, Artificial Sequence
  Description of Artificial Sequence: Synthetic polynucleotide
taattttctcagcagcagccacaactccgaggaacccctttgaaagtcctttcatgaatacatccacggctaaacttctcctttacaccacactgtcgtcgaatggccactcccagtatatataaatttttggagcaggttttctgacttcggtcggaaaacccctcc

SEQ ID NO: 93 - DNA, 168 bp, Artificial Sequence
  Description of Artificial Sequence: Synthetic polynucleotide
taattttctcagcagcagccacaactccgaggaacccctttgaaagtcctttcatgaatacatccacggctaaacttctcctttacaccacactggaaaacataaaatacactttgaatatataaatttttggagcaggttttctgacttcggtcggaaaacccctcc

SEQ ID NO: 94 - DNA, 155 bp, Artificial Sequence
  Description of Artificial Sequence: Synthetic polynucleotide
taatgccacaactccgaggaacccctttgaaagtcctttcacgaatacatccacggctaaacttctcctttacaccacactgtcgtcgaatggccactcccagtatatataaatttttggagcaggttttctgacttcggtcggaaaacccctcc

SEQ ID NO: 95 - DNA, 155 bp, Artificial Sequence
  Description of Artificial Sequence: Synthetic polynucleotide
taatgccacaactccgaggaacccctttgaaagtcctttcacgaatacatccacggctaaacttctcctttacaccacactggaaaacataaaatacactttgaatatataaatttttggagcaggttttctgacttcggtcggaaaacccctcc

SEQ ID NO: 96 - DNA, 198 bp, Artificial Sequence
  Description of Artificial Sequence: Synthetic polynucleotide
taattttctcagcagcagccacaactccgaggaacccctttgaaagtcctttcatgaatacatccacggctaaacttctcctttacaccacattagccagaaggcttgaaggcaaggcgtgagggagcgcccaggacgctctcggagatatataaatttttggagcaggttttctgacttcggtcggaaaacccctcc

SEQ ID NO: 97 - DNA, 223 bp, Artificial Sequence
  Description of Artificial Sequence: Synthetic polynucleotide
taattttctcagcagcagccacaactccgaggaacccctttgaaagtcctttcatgaatacatccacggctaaacttctcctttacaccacactggaaaacataaaatacactttgattagccagaaggcttgaaggcaaggcgtgagggagcgcccaggacgctctcggagatatataaatttttggagcaggttttctgacttcggtcggaaaacccctcc

SEQ ID NO: 98 - DNA, 211 bp, Artificial Sequence
  Description of Artificial Sequence: Synthetic polynucleotide
taattttctcagcagcagccacaactccgaggaacccctttgaaagtcctttcatgaatacatccacggctaaacttctcctttacaccacattagcaaaggcagaaggcttgaaggcaaggacaccgggagcgcccaggacgctctcggaggggccgggatatataaatttttggagcaggttttctgacttcggtcggaaaacccctcc

SEQ ID NO: 99 - DNA, 236 bp, Artificial Sequence
  Description of Artificial Sequence: Synthetic polynucleotide
taattttctcagcagcagccacaactccgaggaacccctttgaaagtcctttcatgaatacatccacggctaaacttctcctttacaccacactggaaaacataaaatacactttgattagcaaaggcagaaggcttgaaggcaaggacaccgggagcgcccaggacgctctcggaggggccgggatatataaatttttggagcaggttttctgacttcggtcggaaaacccctcc

SEQ ID NO: 100 - DNA, 222 bp, Artificial Sequence
  Description of Artificial Sequence: Synthetic polynucleotide
taataccctgtttggtttctcagcagcaggggagtggccctccttggcctttgaaagtcctttcatgaatacatccacggctaaacttctcctttacaccacattagcaaaggcagaaggcttgaaggcaaggacaccgggagcgcccaggacgctctcggaggggccgggatatataaatttttggagcaggttttctgacttcggtcggaaaacccctcc

SEQ ID NO: 101 - DNA, 247 bp, Artificial Sequence
  Description of Artificial Sequence: Synthetic polynucleotide
taataccctgtttggtttctcagcagcaggggagtggccctccttggcctttgaaagtcctttcatgaatacatccacggctaaacttctcctttacaccacactggaaaacataaaatacactttgattagcaaaggcagaaggcttgaaggcaaggacaccgggagcgcccaggacgctctcggaggggccgggatatataaatttttggagcaggttttctgacttcggtcggaaaacccctcc

SEQ ID NO: 102 - RNA, 150 nt, Artificial Sequence
  Description of Artificial Sequence: Synthetic polynucleotide
accgauggguauggccucuaaaaacauggccccagcagcuucagucccuuucucgucgauggucagcacagccuuaugcacggccuggaggggagagaagcagaguggaauuuuuggagcagguuuucugacuucggucggaaaaccccu

SEQ ID NO: 103 - RNA, 150 nt, Artificial Sequence
  Description of Artificial Sequence: Synthetic polynucleotide
accgauggguauggccucuaaaaacauggccccagcagcuucagucccuuacucgucgauggucagcacagccuuaugcacggccuggaggggagagaagcagaguggaauuuuuggagcagguuuucugacuucggucggaaaaccccu

SEQ ID NO: 104 - DNA, 118 bp, Artificial Sequence
  Description of Artificial Sequence: Synthetic polynucleotide
tagggatagggatagggacaactccctccttggcctttgaaagtcctttcatgaatacatccacggctaatgaattcctttacaccacactgtcgtcgaatggccactcccagttctc

SEQ ID NO: 105 - DNA, 134 bp, Artificial Sequence
  Description of Artificial Sequence: Synthetic polynucleotide
tagggatagggatagggacaactccctccttggcctttgaaagtcctttcatgaatacatccacggctaatgtggaatagtataacaatatgctaaatgttgttatagtatcccacgaattcctttacaccaca

SEQ ID NO: 106 - DNA, 146 bp, Artificial Sequence
  Description of Artificial Sequence: Synthetic polynucleotide
caactccctccttggcctttgaaagtcctttcatgaatacaccctatgatattgttgtaaatcgtataacaatatgataaggtgcatccacggctaatgaattcctttacaccacactgtcgtcgaatggccactcccagttctca

SEQ ID NO: 107 - DNA, 119 bp, Artificial Sequence
  Description of Artificial Sequence: Synthetic polynucleotide
tagggatagggatagggacaactccctccttggcctttgaaagtcctttcatgaatacatccatagctaatgaattcctttacaccacactgtcgtcgaatggccactcccagttctca

SEQ ID NO: 108 - DNA, 117 bp, Artificial Sequence
  Description of Artificial Sequence: Synthetic polynucleotide
tagggatagggatagggaccctgtttggttctctcagcagcagccacaactccctccttggcctttgaaagtcctttcatgaatacatccatagctaatgaattcctttacaccaca

SEQ ID NO: 109 - DNA, 45 bp, Unknown
  Description of Unknown: target sequence
tacagcagtactgagcaatgctgtagtcagcaatctttgcaatga

SEQ ID NO: 110 - DNA, 45 bp, Unknown
  Description of Unknown: target sequence
tacagcagtactgagcaatgccgtagtcagcaatctttgcaatga

SEQ ID NO: 111 - DNA, 45 bp, Artificial Sequence
  Description of Artificial Sequence: Synthetic oligonucleotide
tacagcagtactgggtggtgccgtagtcagcaatctttgcaatga

SEQ ID NO: 112 - DNA, 45 bp, Artificial Sequence
  Description of Artificial Sequence: Synthetic oligonucleotide
tacagcaggactgagcagtgccgtagtgtcgaatctttgcaatga

SEQ ID NO: 113 - DNA, 45 bp, Artificial Sequence
  Description of Artificial Sequence: Synthetic oligonucleotide
taaagcaggactgagtagtgccgtagtcagcaatctttgcaatga

SEQ ID NO: 114 - DNA, 45 bp, Artificial Sequence
  Description of Artificial Sequence: Synthetic oligonucleotide
tacagcagtactgagcagtgccgtagtgtcgtttctttgcaatga

SEQ ID NO: 115 - DNA, 45 bp, Artificial Sequence
  Description of Artificial Sequence: Synthetic oligonucleotide
tacagcattactgagcagtgccgtagtgtcgtttctttgcaatga

SEQ ID NO: 116 - DNA, 45 bp, Artificial Sequence
  Description of Artificial Sequence: Synthetic oligonucleotide
tacagcaggactgagtagtgccgtagtgtcgaatctttgcaatga

SEQ ID NO: 117 - DNA, 45 bp, Artificial Sequence
  Description of Artificial Sequence: Synthetic oligonucleotide
tacagcaggactgagcggtgccgtagtcagcaatctttgcaatga

SEQ ID NO: 118 - DNA, 45 bp, Artificial Sequence
  Description of Artificial Sequence: Synthetic oligonucleotide
tacagcagtactgagcggtgccgtagtcagcaatctttgcaatga

SEQ ID NO: 119 - DNA, 45 bp, Artificial Sequence
  Description of Artificial Sequence: Synthetic oligonucleotide
tacagcaggactgggtgatgccgtagtcagcaatctttgcaatga

SEQ ID NO: 120 - DNA, 45 bp, Artificial Sequence
  Description of Artificial Sequence: Synthetic oligonucleotide
taaagcagtactgagcggtgccgtagtcagcaatctttgcaatga

SEQ ID NO: 121 - DNA, 45 bp, Artificial Sequence
  Description of Artificial Sequence: Synthetic oligonucleotide
tacagcagtcctgggtggtgccgtagtcagcaatctttgcaatga

SEQ ID NO: 122 - DNA, 45 bp, Artificial Sequence
  Description of Artificial Sequence: Synthetic oligonucleotide
tacagcaggactgggtagtgccgtagtgtcgaatctttgcaatga

SEQ ID NO: 123 - DNA, 45 bp, Artificial Sequence
  Description of Artificial Sequence: Synthetic oligonucleotide
tacagcaggactgagcagtgccgtagtcagcaatctttgcaatga

SEQ ID NO: 124 - DNA, 45 bp, Artificial Sequence
  Description of Artificial Sequence: Synthetic oligonucleotide
taaagcagtactgagcagtgccgtagtcagcaatctttgcaatga

SEQ ID NO: 125 - DNA, 45 bp, Artificial Sequence
  Description of Artificial Sequence: Synthetic oligonucleotide
taaagcaggcctgagcagtgccgtagtgtcgaatctttgcaatga

SEQ ID NO: 126 - DNA, 45 bp, Artificial Sequence
  Description of Artificial Sequence: Synthetic oligonucleotide
taaagcaggactgggcagtgccgtagtgtcgaatctttgcaatga

SEQ ID NO: 127 - DNA, 45 bp, Artificial Sequence
  Description of Artificial Sequence: Synthetic oligonucleotide
tacagcaggactgggcgatgccgtagtcagcaatctttgcaatga

SEQ ID NO: 128 - DNA, 45 bp, Artificial Sequence
  Description of Artificial Sequence: Synthetic oligonucleotide
tacagcaggactgggcagtgccgtagtcagcaatctttgcaatga

SEQ ID NO: 129 - DNA, 45 bp, Artificial Sequence
  Description of Artificial Sequence: Synthetic oligonucleotide
taaagcaggactgggcagtgccgtagtcagcaatctttgcaatga

SEQ ID NO: 130 - DNA, 45 bp, Artificial Sequence
  Description of Artificial Sequence: Synthetic oligonucleotide
tacagcattacggagcagtgccgtagtgtcgtttctttgcaatga

SEQ ID NO: 131 - DNA, 45 bp, Artificial Sequence
  Description of Artificial Sequence: Synthetic oligonucleotide
tacagcaggcctgagcagtgccgtagtgtcgaatctttgcaatga

SEQ ID NO: 132 - DNA, 45 bp, Artificial Sequence
  Description of Artificial Sequence: Synthetic oligonucleotide
taaagcaggactgggtagtgccgtagtgtcgaatctttgcaatga

SEQ ID NO: 133 - DNA, 45 bp, Artificial Sequence
  Description of Artificial Sequence: Synthetic oligonucleotide
tacagcagtactgagcagtgccgtagtgtcgaatctttgcaatga

SEQ ID NO: 134 - DNA, 45 bp, Artificial Sequence
  Description of Artificial Sequence: Synthetic oligonucleotide
tacagcaataccgagcagtgccgtagtcagcaatctttgcaatga

SEQ ID NO: 135 - DNA, 45 bp, Artificial Sequence
  Description of Artificial Sequence: Synthetic oligonucleotide
tacagcagtactgggcggtgccgtagtcagcaatctttgcaatga

SEQ ID NO: 136 - DNA, 45 bp, Artificial Sequence
  Description of Artificial Sequence: Synthetic oligonucleotide
tacagcaggactgggtggtgccgtagtcagcaatctttgcaatga

SEQ ID NO: 137 - DNA, 45 bp, Artificial Sequence
  Description of Artificial Sequence: Synthetic oligonucleotide
taaagcaggactgagcagtgccgtagtcagcaatctttgcaatga

SEQ ID NO: 138 - DNA, 45 bp, Artificial Sequence
  Description of Artificial Sequence: Synthetic oligonucleotide
taaagcaggactgagtagtgccgtagtgtcgaatctttgcaatga

SEQ ID NO: 139 - DNA, 45 bp, Artificial Sequence
  Description of Artificial Sequence: Synthetic oligonucleotide
tacagcagtactgagtggtgccgtagtcagcaatctttgcaatga

SEQ ID NO: 140 - DNA, 45 bp, Artificial Sequence
  Description of Artificial Sequence: Synthetic oligonucleotide
tacagcaggactgggcggtgccgtagtcagcaatctttgcaatga

SEQ ID NO: 141 - DNA, 45 bp, Artificial Sequence
  Description of Artificial Sequence: Synthetic oligonucleotide
tacagcagtcctgggcggtgccgtagtcagcaatctttgcaatga

SEQ ID NO: 142 - DNA, 45 bp, Artificial Sequence
  Description of Artificial Sequence: Synthetic oligonucleotide
taaagcagtactgagcagtgccgtagtgtcgaatctttgcaatga

SEQ ID NO: 143 - DNA, 45 bp, Artificial Sequence
  Description of Artificial Sequence: Synthetic oligonucleotide
taaagcaggactgagcagtgccgtagtgtcgaatctttgcaatga

SEQ ID NO: 144 - DNA, 45 bp, Artificial Sequence
  Description of Artificial Sequence: Synthetic oligonucleotide
tacagcaggactgggcagtgccgtagtgtcgaatctttgcaatga

SEQ ID NO: 145 - DNA, 63 bp, Artificial Sequence
  Description of Artificial Sequence: Synthetic oligonucleotide
gctaaatcgattctaaagcaggacggggtggtgccgtagtgtcgtatctttgcacttacagtc

SEQ ID NO: 146 - DNA, 63 bp, Artificial Sequence
  Description of Artificial Sequence: Synthetic oligonucleotide
gctaaatcgattctaaagcaggactgggtggtgccgtagtgtcgtatctttgcacttacagtc

SEQ ID NO: 147 - DNA, 63 bp, Artificial Sequence
  Description of Artificial Sequence: Synthetic oligonucleotide
gctaaatcgattctaaagcaggaccgggtggtgccgtagtgtcgtatctttgcacttacagtc

SEQ ID NO: 148 - DNA, 63 bp, Artificial Sequence
  Description of Artificial Sequence: Synthetic oligonucleotide
gctaaatcgattctagagcaggactgggtggtgccgtagtgtcgtatctttgcacttacagtc

SEQ ID NO: 149 - DNA, 63 bp, Artificial Sequence
  Description of Artificial Sequence: Synthetic oligonucleotide
gctaaatcgattctacagcaggactgggtggtgccgtagtgtcgtatctttgcacttacagtc

SEQ ID NO: 150 - DNA, 63 bp, Artificial Sequence
  Description of Artificial Sequence: Synthetic oligonucleotide
gctaaatcgattctaaagcatgactgggtggtgccgtagtcagcaatctttgcacttacagtc

SEQ ID NO: 151 - DNA, 63 bp, Artificial Sequence
  Description of Artificial Sequence: Synthetic oligonucleotide
gctaaatcgattctaaagcaggactgggtggtgccgtagtcagcaatctttgcacttacagtc

SEQ ID NO: 152 - DNA, 63 bp, Artificial Sequence
  Description of Artificial Sequence: Synthetic oligonucleotide
gctaaatcgattctaaagcagtactgggtggtgccgtagtcagcaatctttgcacttacagtc

SEQ ID NO: 153 - DNA, 63 bp, Artificial Sequence
  Description of Artificial Sequence: Synthetic oligonucleotide
gctaaatcgattctagagcatgactgggtggtgccgtagtcagcaatctttgcacttacagtc

SEQ ID NO: 154 - DNA, 63 bp, Artificial Sequence
  Description of Artificial Sequence: Synthetic oligonucleotide
gctaaatcgattctagagcaggacggggtggtgccgtagtcagcaatctttgcacttacagtc

SEQ ID NO: 155 - DNA, 272 bp, Artificial Sequence
  Description of Artificial Sequence: Synthetic polynucleotide
ccctggtgtgccctctgatgttttttaggggattctacagtaggactgagcactgccgagctgggcaatctttgcatactacgcagcattgggatacagtgtgaaaagcagcaccctggtgtgccctctgatgttttttaggggattctacagtaggactgagcactgccgagctgggcaatctttgcatactacgcagcattgggatacagtgtgaaaagcagcagtggaatttttggagcaggttttctgacttcggtcggaaaacccct

SEQ ID NO: 156 - DNA, 272 bp, Artificial Sequence
  Description of Artificial Sequence: Synthetic polynucleotide
ccctggtgtgccctctgatgttttttaggggattctacagcagtacagaggactgccgaggtcaccaatctttgcatactacgcagcattgggatacagtgtgaaaagcagcaccctggtgtgccctctgatgttttttaggggattctacagcagtacagaggactgccgaggtcaccaatctttgcatactacgcagcattgggatacagtgtgaaaagcagcagtggaatttttggagcaggttttctgacttcggtcggaaaacccct

SEQ ID NO: 157 - DNA, 272 bp, Artificial Sequence
  Description of Artificial Sequence: Synthetic polynucleotide
ccctggtgtgccctctgatgttttttaggggattctacaacagtactgagctatcccgaattcaacaatctttgcatactacgcagcattgggatacagtgtgaaaagcagcaccctggtgtgccctctgatgttttttaggggattctacaacagtactgagctatcccgaattcaacaatctttgcatactacgcagcattgggatacagtgtgaaaagcagcagtggaatttttggagcaggttttctgacttcggtcggaaaacccct

SEQ ID NO: 158 - DNA, 272 bp, Artificial Sequence
  Description of Artificial Sequence: Synthetic polynucleotide
ccctggtgtgccctctgatgttttttaggggcttctacagcagttcggaggaatcccgaggtcagcaatctttgcatactacgcagcattgggatacagtgtgaaaagcagcaccctggtgtgccctctgatgttttttaggggcttctacagcagttcggaggaatcccgaggtcagcaatctttgcatactacgcagcattgggatacagtgtgaaaagcagcagtggaatttttggagcaggttttctgacttcggtcggaaaacccct

SEQ ID NO: 159 - DNA, 270 bp, Artificial Sequence
  Description of Artificial Sequence: Synthetic polynucleotide
ccctggtgtgccctctgatgttttttaggggattctacggcggtactgaccaatcccgtagttagcaatctttgcatactacgcagcattgggatacagtgtgaaaagcagcccctggtgtgccctctgatgttttttaggggattctacggcggtactgaccaatcccgtagttagcaatctttgcatactacgcagcattgggatacagtgtgaaaagcagcgtggaatttttggagcaggttttctgacttcggtcggaaaacccct

SEQ ID NO: 160 - DNA, 272 bp, Artificial Sequence
  Description of Artificial Sequence: Synthetic polynucleotide
ccctggtgtgccctctgatgttttttaggggattctaccgcagtactacccgatcccgtagtcagcaatctttgcatactacgcagcattgggatacagtgtgaaaagcagcaccctggtgtgccctctgatgttttttaggggattctaccgcagtactacccgatcccgtagtcagcaatctttgcatactacgcagcattgggatacagtgtgaaaagcagcagtggaatttttggagcaggttttctgacttcggtcggaaaacccct

SEQ ID NO: 161 - DNA, 272 bp, Artificial Sequence
  Description of Artificial Sequence: Synthetic polynucleotide
ccctggtgtgccctctgatgttttttaggggattctacggcagttcatagcaatcccgtagtcaacaatctttgcctactacgcagcattgggatacagtgtgaaaagcagcaccctggtgtgccctctgatgttttttaggggattctacggcagttcatagcaatcccgtagtcaacaatctttgcctactacgcagcattgggatacagtgtgaaaagcagcagtggaatttttggagcaggttttctgacttcggtcggaaaacccct

SEQ ID NO: 162 - DNA, 45 bp, Artificial Sequence
  Description of Artificial Sequence: Synthetic oligonucleotide
attctacagcacgactgagcaatgccgtattcagcaatctttgca

SEQ ID NO: 163 - DNA, 45 bp, Artificial Sequence
  Description of Artificial Sequence: Synthetic oligonucleotide
attctagagcagtactgagcaatgccgtggttaccaatctttgca

SEQ ID NO: 164 - DNA, 45 bp, Artificial Sequence
  Description of Artificial Sequence: Synthetic oligonucleotide
attctacagcagtagtaagctttgccgaagtgagcaatctttaca

SEQ ID NO: 165 - DNA, 45 bp, Artificial Sequence
  Description of Artificial Sequence: Synthetic oligonucleotide
attctacagcacgactgagcaaggccgtagtcagcgatctttgca

SEQ ID NO: 166 - DNA, 45 bp, Artificial Sequence
  Description of Artificial Sequence: Synthetic oligonucleotide
attctacagcactacggggcaatgccgaagtctgcaatctttgca

SEQ ID NO: 167 - DNA, 45 bp, Artificial Sequence
  Description of Artificial Sequence: Synthetic oligonucleotide
attctacggcggtactgaccaatcccgtagttagcaatctttgca

SEQ ID NO: 168 - DNA, 45 bp, Artificial Sequence
  Description of Artificial Sequence: Synthetic oligonucleotide
attctacagcggtactcatcaatgccgtagtcgtcaatttttgca

SEQ ID NO: 169 - DNA, 45 bp, Artificial Sequence
  Description of Artificial Sequence: Synthetic oligonucleotide
attctaaagccgtactaagcagtaccgtagtccccaatctttgca

SEQ ID NO: 170 - DNA, 45 bp, Artificial Sequence
  Description of Artificial Sequence: Synthetic oligonucleotide
attctagagccatactgttcaatgccgaagtcagcaatctttgca

SEQ ID NO: 171 - DNA, 45 bp, Artificial Sequence
  Description of Artificial Sequence: Synthetic oligonucleotide
attctacagcggtactgggcaatgccgtggtctgcaatctttgca

SEQ ID NO: 172 - DNA, 45 bp, Artificial Sequence
  Description of Artificial Sequence: Synthetic oligonucleotide
attctacagctgtactgaggaatgccgtctttaccaatctttgca

SEQ ID NO: 173 - DNA, 45 bp, Artificial Sequence
  Description of Artificial Sequence: Synthetic oligonucleotide
attctacagcagtacttatcagtgccgttgttagcaatctttgca

SEQ ID NO: 174 - DNA, 45 bp, Artificial Sequence
  Description of Artificial Sequence: Synthetic oligonucleotide
attctatagctggactgagctctgccgtagtcgtcaatctttgca

SEQ ID NO: 175 - DNA, 45 bp, Artificial Sequence
  Description of Artificial Sequence: Synthetic oligonucleotide
attctacagcaatactcagcagtgccgtagtcaccaatctttgca

SEQ ID NO: 176 - DNA, 45 bp, Artificial Sequence
  Description of Artificial Sequence: Synthetic oligonucleotide
attctacaacagtactgcgcaccgccgtagtctgcaatctttgca

SEQ ID NO: 177 - DNA, 45 bp, Artificial Sequence
  Description of Artificial Sequence: Synthetic oligonucleotide
attctagagccgtactgcgctttgccgtagtcatcaatctttgca

SEQ ID NO: 178 - DNA, 45 bp, Artificial Sequence
  Description of Artificial Sequence: Synthetic oligonucleotide
attctacagccgtactgcccaatgccgaagtaaccaatctttgca

SEQ ID NO: 179 - DNA, 45 bp, Artificial Sequence
  Description of Artificial Sequence: Synthetic oligonucleotide
attctaccgtgggactgagcagtcccgtagtctgcaatctttgca

SEQ ID NO: 180 - DNA, 45 bp, Artificial Sequence
  Description of Artificial Sequence: Synthetic oligonucleotide
attctacagcagtactgttcagtgcgctagtaagcaatctttgca

SEQ ID NO: 181 - DNA, 45 bp, Artificial Sequence
  Description of Artificial Sequence: Synthetic oligonucleotide
attctacaggtgtactgttctatgccgttgttagccatctttgca

SEQ ID NO: 182 - DNA, 45 bp, Artificial Sequence
  Description of Artificial Sequence: Synthetic oligonucleotide
attctagggcagtactgagagatgccgatgtcagcaatctttgca

SEQ ID NO: 183 - DNA, 45 bp, Artificial Sequence
  Description of Artificial Sequence: Synthetic oligonucleotide
attctacagcagtattgggcagtcccgtagtcagcaatctttgca

SEQ ID NO: 184 - DNA, 45 bp, Artificial Sequence
  Description of Artificial Sequence: Synthetic oligonucleotide
attctacagcaggactgggcgatcccgtagtcatcaatctttgca

SEQ ID NO: 185 - DNA, 45 bp, Artificial Sequence
  Description of Artificial Sequence: Synthetic oligonucleotide
attctacagcagtactaagctatggcgtacgcaccaatctttgca

SEQ ID NO: 186 - DNA, 45 bp, Artificial Sequence
  Description of Artificial Sequence: Synthetic oligonucleotide
attctacagtaggactgagcactgccgagctgggcaatctttgca

SEQ ID NO: 187 - DNA, 45 bp, Artificial Sequence
  Description of Artificial Sequence: Synthetic oligonucleotide
attctaccgcagtactgagcaaagccgaggtaaacaatctttgca

SEQ ID NO: 188 - DNA, 45 bp, Artificial Sequence
  Description of Artificial Sequence: Synthetic oligonucleotide
attctacagcagtactgagcaattccgttaaccgcaatctttgca

SEQ ID NO: 189 - DNA, 45 bp, Artificial Sequence
  Description of Artificial Sequence: Synthetic oligonucleotide
attctacagtagtccggagctattccgtagtccgcaatctttgca

SEQ ID NO: 190 - DNA, 45 bp, Artificial Sequence
  Description of Artificial Sequence: Synthetic oligonucleotide
attctacagcaggactgagcaatggagtagtcggcaatctttgca

SEQ ID NO: 191 - DNA, 45 bp, Artificial Sequence
  Description of Artificial Sequence: Synthetic oligonucleotide
attctaccgcagtactacccgatcccgtagtcagcaatctttgca

SEQ ID NO: 192 - DNA, 45 bp, Artificial Sequence
  Description of Artificial Sequence: Synthetic oligonucleotide
attctacaggagttctgagcattcccgtcgtcatcaatctttgca

SEQ ID NO: 193 - DNA, 45 bp, Artificial Sequence
  Description of Artificial Sequence: Synthetic oligonucleotide
attctacagcagtacagaggactgccgaggtcaccaatctttgca

SEQ ID NO: 194 - DNA, 45 bp, Artificial Sequence
  Description of Artificial Sequence: Synthetic oligonucleotide
attctacagaaggaccgagcagtcccgtagtcagcaatctttgca

SEQ ID NO: 195 - DNA, 45 bp, Artificial Sequence
  Description of Artificial Sequence: Synthetic oligonucleotide
attctacagcattactgagcaatcccgtattaagcaatctttgca

SEQ ID NO: 196 - DNA, 45 bp, Artificial Sequence
  Description of Artificial Sequence: Synthetic oligonucleotide
attctacagcagtacgaagcaatcccgccgttagcaatctttgca

SEQ ID NO: 197 - DNA, 45 bp, Artificial Sequence
  Description of Artificial Sequence: Synthetic oligonucleotide
attctacagcagtactgttcaattccgatgtaagcaatctttgca

SEQ ID NO: 198 - DNA, 45 bp, Artificial Sequence
  Description of Artificial Sequence: Synthetic oligonucleotide
attctacaacagtactgagctatcccgaattcaacaatctttgca

SEQ ID NO: 199 - DNA, 45 bp, Artificial Sequence
  Description of Artificial Sequence: Synthetic oligonucleotide
attctacggcagttcatagcaatcccgtagtcaacaatctttgcc

SEQ ID NO: 200 - DNA, 45 bp, Artificial Sequence
  Description of Artificial Sequence: Synthetic oligonucleotide
cttctacagcagttcggaggaatcccgaggtcagcaatctttgca

SEQ ID NO: 201 - DNA, 45 bp, Artificial Sequence
  Description of Artificial Sequence: Synthetic oligonucleotide
attctacagctgtactaggctatcccgtagtccgcaatctttgca

SEQ ID NO: 202 - DNA, 45 bp, Artificial Sequence
  Description of Artificial Sequence: Synthetic oligonucleotide
attctacagccgtactgggaaatcccgtagtctgcaatctttgca

SEQ ID NO: 203 - DNA, 45 bp, Artificial Sequence
  Description of Artificial Sequence: Synthetic oligonucleotide
attctacagaagtacagagacatcccgtagttaacaatcttcgca

SEQ ID NO: 204 - DNA, 45 bp, Artificial Sequence
  Description of Artificial Sequence: Synthetic oligonucleotide
attctacagccgtactgagctatcccgtagtccgcaatctttgca

SEQ ID NO: 205 - DNA, 45 bp, Artificial Sequence
  Description of Artificial Sequence: Synthetic oligonucleotide
attctactgcagcaccgagcaatcccgcattccccaatctttgca

SEQ ID NO: 206 - DNA, 45 bp, Artificial Sequence
  Description of Artificial Sequence: Synthetic oligonucleotide
attctacagcagtcctgagcagtaccgtagtcagcaatctttgca

SEQ ID NO: 207 - DNA, 45 bp, Artificial Sequence
  Description of Artificial Sequence: Synthetic oligonucleotide
attctaaggcagtactgagccgcgccgtagtcggcaatctttgca

SEQ ID NO: 208 - DNA, 45 bp, Artificial Sequence
  Description of Artificial Sequence: Synthetic oligonucleotide
gaaagtactttgatgaatacatccacggctaatgaattcctttac

SEQ ID NO: 209 - DNA, 45 bp, Artificial Sequence
  Description of Artificial Sequence: Synthetic oligonucleotide
gaaagtcctttcaagaatacatccacggctatactattcctttac

SEQ ID NO: 210 - DNA, 45 bp, Artificial Sequence
  Description of Artificial Sequence: Synthetic oligonucleotide
gaaagtcctttgatgcatacatccacggctaatgaattcctttac

SEQ ID NO: 211 - DNA, 45 bp, Artificial Sequence
  Description of Artificial Sequence: Synthetic oligonucleotide
gaaagtcctttgatgcatacatccacggctatactattcctttac

SEQ ID NO: 212 - DNA, 45 bp, Artificial Sequence
  Description of Artificial Sequence: Synthetic oligonucleotide
gaaagtcctttgaggcatacatccacggctatactattcctttac

SEQ ID NO: 213 - DNA, 45 bp, Artificial Sequence
  Description of Artificial Sequence: Synthetic oligonucleotide
gaaagtcctttgaggaatacatccacggctatactattcctttac

SEQ ID NO: 214 - DNA, 45 bp, Artificial Sequence
  Description of Artificial Sequence: Synthetic oligonucleotide
gaaagtcctttgaagcatacatccacggctatactattcctttac

SEQ ID NO: 215 - DNA, 45 bp, Artificial Sequence
  Description of Artificial Sequence: Synthetic oligonucleotide
gaaagtactttcacgaatacatccacggctatactattcctttac

SEQ ID NO: 216 - DNA, 45 bp, Artificial Sequence
  Description of Artificial Sequence: Synthetic oligonucleotide
gaaagtcctttcacgcatacatccacggctatactattcctttac

SEQ ID NO: 217 - DNA, 45 bp, Artificial Sequence
  Description of Artificial Sequence: Synthetic oligonucleotide
gaaagtcctttcaagcatacatccacggctatactattcctttac

SEQ ID NO: 218 - DNA, 45 bp, Artificial Sequence
  Description of Artificial Sequence: Synthetic oligonucleotide
cccacctctccagggcgaatttggacatacagcctgtccactgct

SEQ ID NO: 219 - DNA, 43 bp, Artificial Sequence
  Description of Artificial Sequence: Synthetic oligonucleotide
cccacctctccgggacgaactcggacaacagttgtccactgct

SEQ ID NO: 220 - DNA, 43 bp, Artificial Sequence
  Description of Artificial Sequence: Synthetic oligonucleotide
cccacctctccaggacgaactcggacaacagatgtccactgct

SEQ ID NO: 221 - DNA, 43 bp, Artificial Sequence
  Description of Artificial Sequence: Synthetic oligonucleotide
cccacctctccggggcgaactcggacaacagttgtccactgct

SEQ ID NO: 222 - DNA, 45 bp, Artificial Sequence
  Description of Artificial Sequence: Synthetic oligonucleotide
cccacctctccagggcgaatttggtcacacagcctgtccactgct

SEQ ID NO: 223 - DNA, 45 bp, Artificial Sequence
  Description of Artificial Sequence: Synthetic oligonucleotide
cccacctctccagggcgaacttggacacacagcctgtccactgct

SEQ ID NO: 224 - DNA, 45 bp, Artificial Sequence
  Description of Artificial Sequence: Synthetic oligonucleotide
cccaccgctccagggcgaacttggtcacacagcctgtccactgct

SEQ ID NO: 225 - DNA, 43 bp, Artificial Sequence
  Description of Artificial Sequence: Synthetic oligonucleotide
cccacctctccgggacgaactcggacaacagatgtccactgct

SEQ ID NO: 226 - DNA, 45 bp, Artificial Sequence
  Description of Artificial Sequence: Synthetic oligonucleotide
cccacctctccaggacgaactcggacacacaggctgtccactgct

SEQ ID NO: 227 - DNA, 45 bp, Artificial Sequence
  Description of Artificial Sequence: Synthetic oligonucleotide
cccacctcttcagggcgatcttggacacacagcctgtccactgct

SEQ ID NO: 228 - DNA, 43 bp, Artificial Sequence
  Description of Artificial Sequence: Synthetic oligonucleotide
cccacctctccaggacgaactcggacaacagctgtccactgct

SEQ ID NO: 229 - DNA, 45 bp, Artificial Sequence
  Description of Artificial Sequence: Synthetic oligonucleotide
cccacctcttcagggcgaacttggacacacagcctgtccactgct

SEQ ID NO: 230 - DNA, 45 bp, Artificial Sequence
  Description of Artificial Sequence: Synthetic oligonucleotide
cccaccgctccagggcgaacttggacatacagcctgtccactgct

SEQ ID NO: 231 - DNA, 43 bp, Artificial Sequence
  Description of Artificial Sequence: Synthetic oligonucleotide
cccacctctccgggacgaactcggacaacagctgtccactgct

SEQ ID NO: 232 - DNA, 45 bp, Artificial Sequence
  Description of Artificial Sequence: Synthetic oligonucleotide
cccaccgctccagggcgaatttggtcatacagcctgtccactgct

SEQ ID NO: 233 - DNA, 45 bp, Artificial Sequence
  Description of Artificial Sequence: Synthetic oligonucleotide
cccaccgctccagggcgaatttggacatacagcctgtccactgct

SEQ ID NO: 234 - DNA, 45 bp, Artificial Sequence
  Description of Artificial Sequence: Synthetic oligonucleotide
ccgacctcttcagggcgatcttggacacacagcctgtccactgct

SEQ ID NO: 235 - DNA, 45 bp, Artificial Sequence
  Description of Artificial Sequence: Synthetic oligonucleotide
cccacctcttcagggcgatcttggacacacaacctgtccactgct

SEQ ID NO: 236 - DNA, 45 bp, Artificial Sequence
  Description of Artificial Sequence: Synthetic oligonucleotide
cccacctctccagggcgaatttggtcatacagcctgtccactgct

SEQ ID NO: 237 - DNA, 43 bp, Artificial Sequence
  Description of Artificial Sequence: Synthetic oligonucleotide
cccacctctccaggacgaactcggacaacagttgtccactgct

SEQ ID NO: 238 - DNA, 45 bp, Artificial Sequence
  Description of Artificial Sequence: Synthetic oligonucleotide
ccgacctctccagggcgatcttggacacacagcctgtccactgct

SEQ ID NO: 239 - DNA, 45 bp, Artificial Sequence
  Description of Artificial Sequence: Synthetic oligonucleotide
ccgacctcttcagggcgatcttggacacacaacctgtccactgct

SEQ ID NO: 240 - DNA, 45 bp, Artificial Sequence
  Description of Artificial Sequence: Synthetic oligonucleotide
cccacctctccagggcgaacttggacatacagcctgtccactgct

SEQ ID NO: 241 - DNA, 45 bp, Artificial Sequence
  Description of Artificial Sequence: Synthetic oligonucleotide
ccgacctcttcagggcgaactcggacacacagcctgtccactgct

SEQ ID NO: 242 - DNA, 45 bp, Artificial Sequence
  Description of Artificial Sequence: Synthetic oligonucleotide
cccacctctccgggacgaactcggacacacaggctgtccactgct

SEQ ID NO: 243 - DNA, 43 bp, Artificial Sequence
  Description of Artificial Sequence: Synthetic oligonucleotide
cccacctctccgggacgaactcggacaacaggtgtccactgct

SEQ ID NO: 244 - DNA, 45 bp, Artificial Sequence
  Description of Artificial Sequence: Synthetic oligonucleotide
cccacctctccagggcgaatttggacacacagcctgtccactgct

SEQ ID NO: 245 - DNA, 46 bp, Artificial Sequence
  Description of Artificial Sequence: Synthetic oligonucleotide
cccaccgctccagggcgaacttatcacatacagcctgtccactgct

SEQ ID NO: 246 - DNA, 45 bp, Artificial Sequence
  Description of Artificial Sequence: Synthetic oligonucleotide
cccacctctccagggcgaacttggtcatacagcctgtccactgct

SEQ ID NO: 247 - DNA, 45 bp, Artificial Sequence
  Description of Artificial Sequence: Synthetic oligonucleotide
cccaccgctccagggcgaatttggacacacagcctgtccactgct

SEQ ID NO: 248 - DNA, 45 bp, Artificial Sequence
  Description of Artificial Sequence: Synthetic oligonucleotide
cccaccgctccagggcgaacttggtcatacagcctgtccactgct

SEQ ID NO: 249 - DNA, 45 bp, Artificial Sequence
  Description of Artificial Sequence: Synthetic oligonucleotide
cccaccgctccagggcgaatttggtcacacagcctgtccactgct

SEQ ID NO: 250 - DNA, 147 bp, Artificial Sequence
  Description of Artificial Sequence: Synthetic polynucleotide
tgccggcaccattcactcccaggaggccaaagcactctccgggacgaactcggacaacagatgtccactgctgggctggaggtgcctggataaatcttggggtggaatttttggagcaggttttctgacttcggtcggaaaacccct

SEQ ID NO: 251 - DNA, 146 bp, Artificial Sequence
  Description of Artificial Sequence: Synthetic polynucleotide
cccagtgagcatcttgaatgtggttgttttgccggcaccattcactcccaggaggccaaagcactctccgggacgaactcggacaacagatgtccactgcgtggaatttttggagcaggttttctgacttcggtcggaaaacccct

SEQ ID NO: 252 - DNA, 146 bp, Artificial Sequence
  Description of Artificial Sequence: Synthetic polynucleotide
tgccggcaccattcactcccaggaggccaaagcactctccgggacgaactcggacacacaggctgtccactgctgggctggaggtgcctggataaatcttgtggaatttttggagcaggttttctgacttcggtcggaaaacccct

SEQ ID NO: 253 - DNA, 146 bp, Artificial Sequence
  Description of Artificial Sequence: Synthetic polynucleotide
cccagtgagcatcttgaatgtggttgttttgccggcaccattcactcccaggaggccaaagcactctccgggacgaactcggacacacaggctgtccactgtggaatttttggagcaggttttctgacttcggtcggaaaacccct

SEQ ID NO: 254 - DNA, 146 bp, Artificial Sequence
  Description of Artificial Sequence: Synthetic polynucleotide
tgccggcaccattcactcccaggaggccaaagcactcttcagggcgaacttggacacacagcctgtccactgctgggctggaggtgcctggataaatcttgtggaatttttggagcaggttttctgacttcggtcggaaaacccct

SEQ ID NO: 255 - DNA, 146 bp, Artificial Sequence
  Description of Artificial Sequence: Synthetic polynucleotide
cccagtgagcatcttgaatgtggttgttttgccggcaccattcactcccaggaggccaaagcactcttcagggcgaacttggacacacagcctgtccactgtggaatttttggagcaggttttctgacttcggtcggaaaacccct

SEQ ID NO: 256 - DNA, 146 bp, Artificial Sequence
  Description of Artificial Sequence: Synthetic polynucleotide
tgccggcaccattcactcccaggaggccaaagcactcttcagggcgatcttggacacacagcctgtccactgctgggctggaggtgcctggataaatcttgtggaatttttggagcaggttttctgacttcggtcggaaaacccct

SEQ ID NO: 257 - DNA, 146 bp, Artificial Sequence
  Description of Artificial Sequence: Synthetic polynucleotide
cccagtgagcatcttgaatgtggttgttttgccggcaccattcactcccaggaggccaaagcactcttcagggcgatcttggacacacagcctgtccactgtggaatttttggagcaggttttctgacttcggtcggaaaacccct

SEQ ID NO: 258 - DNA, 146 bp, Artificial Sequence
  Description of Artificial Sequence: Synthetic polynucleotide
tgccggcaccattcactcccaggaggccaaagcacgctccagggcgaacttggacatacagcctgtccactgctgggctggaggtgcctggataaatcttgtggaatttttggagcaggttttctgacttcggtcggaaaacccct

SEQ ID NO: 259 - DNA, 146 bp, Artificial Sequence
  Description of Artificial Sequence: Synthetic polynucleotide
cccagtgagcatcttgaatgtggttgttttgccggcaccattcactcccaggaggccaaagcacgctccagggcgaacttggacatacagcctgtccactgtggaatttttggagcaggttttctgacttcggtcggaaaacccct

SEQ ID NO: 260 - DNA, 146 bp, Artificial Sequence
  Description of Artificial Sequence: Synthetic polynucleotide
tgccggcaccattcactcccaggaggccaaagcactctccagggcgaacttggtcatacagcctgtccactgctgggctggaggtgcctggataaatcttgtggaatttttggagcaggttttctgacttcggtcggaaaacccct

SEQ ID NO: 261 - DNA, 146 bp, Artificial Sequence
  Description of Artificial Sequence: Synthetic polynucleotide
cccagtgagcatcttgaatgtggttgttttgccggcaccattcactcccaggaggccaaagcactctccagggcgaacttggtcatacagcctgtccactgtggaatttttggagcaggttttctgacttcggtcggaaaacccct

SEQ ID NO: 262 - DNA, 146 bp, Artificial Sequence
  Description of Artificial Sequence: Synthetic polynucleotide
cccagtgagcatcttgaatgtggttgttttgccggcaccattcactcccaggaggccaaagcactctccgggacgaactcggacaacagttgtccactgcgtggaatttttggagcaggttttctgacttcggtcggaaaacccct

SEQ ID NO: 263 - DNA, 146 bp, Artificial Sequence
  Description of Artificial Sequence: Synthetic polynucleotide
cccagtgagcatcttgaatgtggttgttttgccggcaccattcactcccaggaggccaaagcactctccgggacgaactcggacaacagctgtccactgcgtggaatttttggagcaggttttctgacttcggtcggaaaacccct

SEQ ID NO: 264 - skipped

SEQ ID NO: 265 - DNA, 146 bp, Artificial Sequence
  Description of Artificial Sequence: Synthetic polynucleotide
cccagtgagcatcttgaatgtggttgttttgccggcaccattcactcccaggaggccaaagcactctccaggacgaactcggacaacagatgtccactgcgtggaatttttggagcaggttttctgacttcggtcggaaaacccct

SEQ ID NO: 266 - DNA, 146 bp, Artificial Sequence
  Description of Artificial Sequence: Synthetic polynucleotide
cccagtgagcatcttgaatgtggttgttttgccggcaccattcactcccaggaggccaaagcactctccaggacgaactcggacaacagctgtccactgcgtggaatttttggagcaggttttctgacttcggtcggaaaacccct

SEQ ID NO: 267 - DNA, 146 bp, Artificial Sequence
  Description of Artificial Sequence: Synthetic polynucleotide
cccagtgagcatcttgaatgtggttgttttgccggcaccattcactcccaggaggccaaagcactctccagggcgaacttggacacacagcctgtccactgtggaatttttggagcaggttttctgacttcggtcggaaaacccct

SEQ ID NO: 268 - DNA, 146 bp, Artificial Sequence
  Description of Artificial Sequence: Synthetic polynucleotide
cccagtgagcatcttgaatgtggttgttttgccggcaccattcactcccaggaggccaaagcacgctccagggcgaatttggacacacagcctgtccactgtggaatttttggagcaggttttctgacttcggtcggaaaacccct

SEQ ID NO: 269 - DNA, 146 bp, Artificial Sequence
  Description of Artificial Sequence: Synthetic polynucleotide
cccagtgagcatcttgaatgtggttgttttgccggcaccattcactcccaggaggccaaagcacgctccagggcgaacttggtcacacagcctgtccactgtggaatttttggagcaggttttctgacttcggtcggaaaacccct

SEQ ID NO: 270 - DNA, 146 bp, Artificial Sequence
  Description of Artificial Sequence: Synthetic polynucleotide
cccagtgagcatcttgaatgtggttgttttgccggcaccattcactcccaggaggccaaagcacgctccagggcgaatttggtcatacagcctgtccactgtggaatttttggagcaggttttctgacttcggtcggaaaacccct

SEQ ID NO: 271 - DNA, 146 bp, Artificial Sequence
  Description of Artificial Sequence: Synthetic polynucleotide
cccagtgagcatcttgaatgtggttgttttgccggcaccattcactcccaggaggccaaagcactctccagggcgaatttggtcatacagcctgtccactgtggaatttttggagcaggttttctgacttcggtcggaaaacccct

SEQ ID NO: 272 - DNA, 146 bp, Artificial Sequence
  Description of Artificial Sequence: Synthetic polynucleotide
cccagtgagcatcttgaatgtggttgttttgccggcaccattcactcccaggaggccaaagcactctccagggcgaacttggacatacagcctgtccactgtggaatttttggagcaggttttctgacttcggtcggaaaacccct

SEQ ID NO: 273 - DNA, 146 bp, Artificial Sequence
  Description of Artificial Sequence: Synthetic polynucleotide
cccagtgagcatcttgaatgtggttgttttgccggcaccattcactcccaggaggccaaagcactctccagggcgaatttggacacacagcctgtccactgtggaatttttggagcaggttttctgacttcggtcggaaaacccct

SEQ ID NO: 274 - DNA, 146 bp, Artificial Sequence
  Description of Artificial Sequence: Synthetic polynucleotide
cccagtgagcatcttgaatgtggttgttttgccggcaccattcactcccaggaggccaaagcacgctccagggcgaacttggtcatacagcctgtccactgtggaatttttggagcaggttttctgacttcggtcggaaaacccct

SEQ ID NO: 275 - DNA, 91 bp, Artificial Sequence
  Description of Artificial Sequence: Synthetic oligonucleotide
cccacctcttcagggcgatcttggacacacagcctgtccactgctgtggaatttttggagcaggttttctgacttcggtcggaaaacccct

SEQ ID NO: 276 - DNA, 91 bp, Artificial Sequence
  Description of Artificial Sequence: Synthetic oligonucleotide
cccacctcttcagggcgatcttggacacacaacctgtccactgctgtggaatttttggagcaggttttctgacttcggtcggaaaacccct

SEQ ID NO: 277 - DNA, 91 bp, Artificial Sequence
  Description of Artificial Sequence: Synthetic oligonucleotide
ccgacctcttcagggcgatcttggacacacaacctgtccactgctgtggaatttttggagcaggttttctgacttcggtcggaaaacccct

SEQ ID NO: 278 - DNA, 146 bp, Artificial Sequence
  Description of Artificial Sequence: Synthetic polynucleotide
cccagtgagcatcttgaatgtggttgttttgccggcaccattcactcccaggaggccaaagcactctccggggcgaactcggacaacagttgtccactgcgtggaatttttggagcaggttttctgacttcggtcggaaaacccct

SEQ ID NO: 279 - DNA, 146 bp, Artificial Sequence; identical to SEQ ID NO: 251
  Description of Artificial Sequence: Synthetic polynucleotide

SEQ ID NO: 280 - DNA, 146 bp, Artificial Sequence
  Description of Artificial Sequence: Synthetic polynucleotide
cccagtgagcatcttgaatgtggttgttttgccggcaccattcactcccaggaggccaaagcactctccaggacgaactcggacaacagttgtccactgcgtggaatttttggagcaggttttctgacttcggtcggaaaacccct

SEQ ID NO: 281 - DNA, 146 bp, Artificial Sequence
  Description of Artificial Sequence: Synthetic polynucleotide
cccagtgagcatcttgaatgtggttgttttgccggcaccattcactcccaggaggccaaagcactctccaggacgaactcggacacacaggctgtccactgtggaatttttggagcaggttttctgacttcggtcggaaaacccct

SEQ ID NO: 282 - skipped

SEQ ID NO: 283 - DNA, 146 bp, Artificial Sequence
  Description of Artificial Sequence: Synthetic polynucleotide
cccagtgagcatcttgaatgtggttgttttgccggcaccattcactcccaggaggccaaagcacgctccagggcgaatttggacatacagcctgtccactgtggaatttttggagcaggttttctgacttcggtcggaaaacccct

SEQ ID NO: 284 - DNA, 146 bp, Artificial Sequence
  Description of Artificial Sequence: Synthetic polynucleotide
cccagtgagcatcttgaatgtggttgttttgccggcaccattcactcccaggaggccaaagcacgctccagggcgaacttatcacatacagcctgtccacgtggaatttttggagcaggttttctgacttcggtcggaaaacccct

SEQ ID NO: 285 - DNA, 146 bp, Artificial Sequence
  Description of Artificial Sequence: Synthetic polynucleotide
cccagtgagcatcttgaatgtggttgttttgccggcaccattcactcccaggaggccaaagcacgctccagggcgaatttggtcacacagcctgtccactgtggaatttttggagcaggttttctgacttcggtcggaaaacccct

SEQ ID NO: 286 - DNA, 146 bp, Artificial Sequence
  Description of Artificial Sequence: Synthetic polynucleotide
cccagtgagcatcttgaatgtggttgttttgccggcaccattcactcccaggaggccaaagcactctccagggcgaatttggtcacacagcctgtccactgtggaatttttggagcaggttttctgacttcggtcggaaaacccct

SEQ ID NO: 287-288 - skipped (2 entries)

SEQ ID NO: 289 - DNA, 146 bp, Artificial Sequence
  Description of Artificial Sequence: Synthetic polynucleotide
cccagtgagcatcttgaatgtggttgttttgccggcaccattcactcccaggaggccaaagcactctccagggcgaatttggacatacagcctgtccactgtggaatttttggagcaggttttctgacttcggtcggaaaacccct

SEQ ID NO: 290 - skipped

SEQ ID NO: 291 - DNA, 146 bp, Artificial Sequence
  Description of Artificial Sequence: Synthetic polynucleotide
cccagtgagcatcttgaatgtggttgttttgccggcaccattcactcccaggaggccaaagcactctccagggcgatcttggacacacagcctgtccactgtggaatttttggagcaggttttctgacttcggtcggaaaacccct

SEQ ID NO: 292 - DNA, 145 bp, Artificial Sequence
  Description of Artificial Sequence: Synthetic polynucleotide
ccccagtgagcatcttgaatgtggttgtattgccggcaccattcactcccaggaggccaaagcactctccagtgagaactcggaccacagcctcccgctgtggaatttttggagcaggttttctgacttcggtcggaaaacccct

SEQ ID NO: 293 - DNA, 146 bp, Artificial Sequence
  Description of Artificial Sequence: Synthetic polynucleotide
ccccagtgagcatcttgaatgtggttgttttgccggcaccattcactcccaggaggccaaagcactctcctctgagaactcggaccacagcctcccgctggtggaatttttggagcaggttttctgacttcggtcggaaaacccct

SEQ ID NO: 294 - DNA, 146 bp, Artificial Sequence
  Description of Artificial Sequence: Synthetic polynucleotide
ccccagtgagcatcttgaatgtggttgttttgccggcaccattcagaggcaggaggccaaagcactctcctctgagaactcggaccacagcctcccgctggtggaatttttggagcaggttttctgacttcggtcggaaaacccct

SEQ ID NO: 295 - DNA, 146 bp, Artificial Sequence
  Description of Artificial Sequence: Synthetic polynucleotide
ccccagtgagcatcttgaatgtggttgttttgcgccgaccattcagaggcaggaggccaaagcactctcctctgagaactcggaccacagcctcccgctggtggaatttttggagcaggttttctgacttcggtcggaaaacccct

SEQ ID NO: 296 - DNA, 146 bp, Artificial Sequence
  Description of Artificial Sequence: Synthetic polynucleotide
ccccagtgagcatcttgaatgtccatgttttgcgccgaccattcagaggcaggaggccaaagcactctcctctgagaactcggaccacagcctcccgctggtggaatttttggagcaggttttctgacttcggtcggaaaacccct

SEQ ID NO: 297 - DNA, 43 bp, Artificial Sequence
  Description of Artificial Sequence: Synthetic oligonucleotide
gccccagcagcttcagttccttaatcgtcgatgtagcacagcc

SEQ ID NO: 298 - DNA, 45 bp, Artificial Sequence
  Description of Artificial Sequence: Synthetic oligonucleotide
attcccatggccccagcagcttcagtccttactcgtcgatggtca

SEQ ID NO: 299 - DNA, 45 bp, Artificial Sequence
  Description of Artificial Sequence: Synthetic oligonucleotide
cctctaaaaacatggctccccacagcttcagtcctttctcgtcga

SEQ ID NO: 300 - DNA, 50 bp, Artificial Sequence
  Description of Artificial Sequence: Synthetic oligonucleotide
atggcctgtgcagcttcagtcccttactcgtcgatggccgtcggagctca

SEQ ID NO: 301 - DNA, 42 bp, Artificial Sequence
  Description of Artificial Sequence: Synthetic oligonucleotide
agtaaaacatgtacgccccagccttcagtcccttctcgtcga

SEQ ID NO: 302 - DNA, 45 bp, Artificial Sequence
  Description of Artificial Sequence: Synthetic oligonucleotide
aaaacaatactacccagcagcttcagtccacctcgtcgatggtca

SEQ ID NO: 303 - DNA, 45 bp, Artificial Sequence
  Description of Artificial Sequence: Synthetic oligonucleotide
ctaatccatggctaccccagcagcttgtccctttctcgtcgatgg

SEQ ID NO: 304 - DNA, 45 bp, Artificial Sequence
  Description of Artificial Sequence: Synthetic oligonucleotide
tgctaaaaacatggtgcagcagcttcagtcctgactcgtcgctgg

SEQ ID NO: 305 - DNA, 45 bp, Artificial Sequence
  Description of Artificial Sequence: Synthetic oligonucleotide
aaaaacatggccccagcagcttcagtccccaactcgtcgatggtc

SEQ ID NO: 306 - DNA, 45 bp, Artificial Sequence
  Description of Artificial Sequence: Synthetic oligonucleotide
aaaacatggccccagcagcttcagtccctgactcgtcgatggtca

SEQ ID NO: 307 - DNA, 44 bp, Artificial Sequence
  Description of Artificial Sequence: Synthetic oligonucleotide
ctaaaaacatggcacagcagcttcagtccctccattatcgatgg

SEQ ID NO: 308 - DNA, 45 bp, Artificial Sequence
  Description of Artificial Sequence: Synthetic oligonucleotide
aactacaaggccccagcagcttcatccctttactcgtcgatgtgg

SEQ ID NO: 309 - DNA, 47 bp, Artificial Sequence
  Description of Artificial Sequence: Synthetic oligonucleotide
ctaaaaacatggctcccagcagcttcatatcctttcctgcttcgagg

SEQ ID NO: 310 - DNA, 45 bp, Artificial Sequence
  Description of Artificial Sequence: Synthetic oligonucleotide
taaaaacatggcccccagttcagtccctaattcttatcgaatggt

SEQ ID NO: 311 - DNA, 45 bp, Artificial Sequence
  Description of Artificial Sequence: Synthetic oligonucleotide
ctaaaaacgccccagcagcttcagtcccacatttctcgtcgatgg

SEQ ID NO: 312 - DNA, 45 bp, Artificial Sequence
  Description of Artificial Sequence: Synthetic oligonucleotide
gccccgtcagttcagtccctgttctcgtcgatgcacagcattgcc

SEQ ID NO: 313 - DNA, 45 bp, Artificial Sequence
  Description of Artificial Sequence: Synthetic oligonucleotide
ctaaaaacccatggcgacagcagcttctccccttctcgtcgatgg

SEQ ID NO: 314 - DNA, 43 bp, Artificial Sequence
  Description of Artificial Sequence: Synthetic oligonucleotide
aagctatggccccagcagcttcaccctttctcgtcgtggatcc

SEQ ID NO: 315 - DNA, 45 bp, Artificial Sequence
  Description of Artificial Sequence: Synthetic oligonucleotide
aaaacatggcaccccagcagcttcagcctttctcgtcgaacgtca

SEQ ID NO: 316 - DNA, 48 bp, Artificial Sequence
  Description of Artificial Sequence: Synthetic oligonucleotide
acatggccccagagcttcagtgccctttctcgtcgatggtcctaagca

SEQ ID NO: 317 - DNA, 44 bp, Artificial Sequence
  Description of Artificial Sequence: Synthetic oligonucleotide
ctctaaaaacatgaccccagcagcttcagtcctccgttatcgat

SEQ ID NO: 318 - DNA, 43 bp, Artificial Sequence
  Description of Artificial Sequence: Synthetic oligonucleotide
acatcaggccccacagcttcagtcccttctcgtcgatggacag

SEQ ID NO: 319 - DNA, 44 bp, Artificial Sequence
  Description of Artificial Sequence: Synthetic oligonucleotide
ccccagcgtcttcagtccctttctcgtcgatggtcgaccagcct

SEQ ID NO: 320 - DNA, 45 bp, Artificial Sequence
  Description of Artificial Sequence: Synthetic oligonucleotide
ggccccagcagcttcagtcccagactcgtcgatggtcagcacaac

SEQ ID NO: 321 - DNA, 45 bp, Artificial Sequence
  Description of Artificial Sequence: Synthetic oligonucleotide
taaaaaacatggccccagcagtcagtccctttactcgtcgatggt

SEQ ID NO: 322 - DNA, 45 bp, Artificial Sequence
  Description of Artificial Sequence: Synthetic oligonucleotide
ggccagcagcttcaccaagtttctcgtcgaatatcagcacagcct

SEQ ID NO: 323 - DNA, 47 bp, Artificial Sequence
  Description of Artificial Sequence: Synthetic oligonucleotide
ctaaaaacatggccccagcagcttcagtcaacttctcgattcgatgg

SEQ ID NO: 324 - DNA, 49 bp, Artificial Sequence
  Description of Artificial Sequence: Synthetic oligonucleotide
tggcgccccagcagcttcatcccttctcgtcgggatgctgtcagcacag

SEQ ID NO: 325 - DNA, 100 bp, Artificial Sequence
  Description of Artificial Sequence: Synthetic polynucleotide
atgggtatggcctctaaaaacatggccccagcagcttcagtccctttctcgtcgatggtcagcacagccttatgcacggcctggaggggagagaagcaga

SEQ ID NO: 326 - DNA, 106 bp, Artificial Sequence
  Description of Artificial Sequence: Synthetic polynucleotide
atgggtatggcctctaaaaacatggccccagcagcttcagtcatacctttctcgtcgatggtcagcatgacagccttatgcacggcctggaggggagagaagcaga

SEQ ID NO: 327 - DNA, 145 bp, Artificial Sequence
  Description of Artificial Sequence: Synthetic polynucleotide
atgggtatggcctctaaaaacatggccccagcagcttcagtccctttctcgtcgatggtccaccctatgatattgttgtaaatcgtataacaatatgataaggtgagcacagccttatgcacggcctggaggggagagaagcaga

SEQ ID NO: 328 - DNA, 150 bp, Artificial Sequence
  Description of Artificial Sequence: Synthetic polynucleotide
tgagcatcttgaatgtggttgtcttgccggcaccattcactcccaggaggccaaagcactctccagggcgaacccagacacacagcctgtccactgctgggctggaggtgcctggataaatcttggttagttcatgtagccttaagatgt

SEQ ID NO: 329 - DNA, 100 bp, Artificial Sequence
  Description of Artificial Sequence: Synthetic polynucleotide
actgtggtgtccccagtgagcatcttgaatgtggttgtcttgccggcaccattcactcccaggaggccaaagcactctccagggcgaacccagacacaca

SEQ ID NO: 330 - DNA, 150 bp, Artificial Sequence
  Description of Artificial Sequence: Synthetic polynucleotide
ttggttaaaatactcttgcctgctacggtggcatcccctgaggtcactgtggtgtccccagtgagcatcttgaatgtggttgtcttgccggcaccattcactcccaggaggccaaagcactctccagggcgaacccagacacacagcctg

SEQ ID NO: 331 - DNA, 200 bp, Artificial Sequence
  Description of Artificial Sequence: Synthetic polynucleotide
cctgaggtcactgtggtgtccccagtgagcatcttgaatgtggttgtcttgccggcaccattcactcccaggaggccaaagcactctccagggcgaacccagacacacagcctgtccactgctgggctggaggtgcctggataaatcttggttagttcatgtagccttaagatgtcagtcttatttccaccagtaataat

SEQ ID NO: 332 - DNA, 100 bp, Artificial Sequence
  Description of Artificial Sequence: Synthetic polynucleotide
tgccggcaccattcactcccaggaggccaaagcactctccagggcgaacccagacacacagcctgtccactgctgggctggaggtgcctggataaatctt

SEQ ID NO: 333 - DNA, 100 bp, Artificial Sequence
  Description of Artificial Sequence: Synthetic polynucleotide
ccccagtgagcatcttgaatgtggttgtattgccggcaccattcactcccaggaggccaaagcactctccagggcgaactcacacacagcctgtccactg

SEQ ID NO: 334 - DNA, 100 bp, Artificial Sequence
  Description of Artificial Sequence: Synthetic polynucleotide
cccagtgagcatcttgaatgtggttgtattgccggcaccattcactcccaggaggccaaagcactctccagggcgaactcgacacacagcctgtccactg

SEQ ID NO: 335 - DNA, 150 bp, Artificial Sequence
  Description of Artificial Sequence: Synthetic polynucleotide
atactcttgcctgctacggtggcatcccctgaggtcactgtggtgtccccagtgagcatcttgaatgtggttgtattgccggcaccattcactcccaggaggccaaagcactctccagtgcgaactcggaccacagcctgtccgctgctg

SEQ ID NO: 336 - DNA, 150 bp, Artificial Sequence
  Description of Artificial Sequence: Synthetic polynucleotide
atactcttgcctgctacggtggcatcccctgaggtcactgtggtgtccccagtgagcatcttgaatgtggttgtattgccggcaccattcactcccaggaggccaaagcactctccagtgcgaactcggacaccagcctgtccgctgctg

SEQ ID NO: 337 - DNA, 100 bp, Artificial Sequence
  Description of Artificial Sequence: Synthetic polynucleotide
ccccagtgagcatcttgaatgtggttgtattgccggcaccattcactcccaggaggccaaagcactctccagggcgaactcggacacacagcctgtccac

SEQ ID NO: 338 - DNA, 100 bp, Artificial Sequence
  Description of Artificial Sequence: Synthetic polynucleotide
cccagtgagcatcttgaatgtggttgtattgccggcaccattcactcccaggaggccaaagcactctccagggcgaactccgacacacagcctgtccact

SEQ ID NO: 339 - DNA, 100 bp, Artificial Sequence
  Description of Artificial Sequence: Synthetic polynucleotide
gccggcaccattcactcccaggaggccaaagcactctccagtgagaactcggaccacagcctcccgctgctgggctggaggtgcctggataaatcttggt

SEQ ID NO: 340 - RNA, 66 nt, Artificial Sequence
  Description of Artificial Sequence: Synthetic oligonucleotide
ccccagugagcaucuugaaugugguuguauugccggcaccauucacucccaggaggccaaagcacu

SEQ ID NO: 341 - DNA, 112 bp, Artificial Sequence
  Description of Artificial Sequence: Synthetic oligonucleotide
  Description of Combined DNA/RNA Molecule: Synthetic oligonucleotide
ccccagugagcaucuugaaugugguuguauugccggcaccauucacucccaggaggccaaagcacugtggaatttttggagcaggttttctgacttcggtcggaaaacccct

SEQ ID NO: 342 - DNA, 45 bp, Artificial Sequence
  Description of Artificial Sequence: Synthetic oligonucleotide
cccacctctccagggcgaacttcgacacacagcctgtccactgct

SEQ ID NO: 343 - DNA, 45 bp, Artificial Sequence
  Description of Artificial Sequence: Synthetic oligonucleotide
cccacctctccagggcgaactccgacacacagcctgtccactgct

SEQ ID NO: 344 - DNA, 146 bp, Artificial Sequence
  Description of Artificial Sequence: Synthetic polynucleotide
gtgccctctgatgtttttatccccattctacagcattacggagcagtgccgtagtgtcgtttctttgcaatgatggcagcattgggatacagtgtgaaaagtggaatttttggagcaggttttctgacttcggtcggaaaacccct

SEQ ID NO: 345 - DNA, 115 bp, Artificial Sequence
  Description of Artificial Sequence: Synthetic polynucleotide
ctggcaacttcaggtgcacgaaaccctggtgtgccctctgatgtttttatccccattctacagcattacggagcagtgccgtagtgtcgtttctttgcaagtggaatttttggag

SEQ ID NO: 346 - DNA, 77 bp, Artificial Sequence
  Description of Artificial Sequence: Synthetic oligonucleotide
gaccatcgacaagaaagggactgaagctgctgatatgctaaatgtcagcagcttcagtccctttctcgtcgatggtc

SEQ ID NO: 347 - DNA, 77 bp, Artificial Sequence
  Description of Artificial Sequence: Synthetic oligonucleotide
gaccatcgacaagaaagggactgaagctgctgatatgctaaatgtcagcagcttcagtcccttactcgtcgatggtc

SEQ ID NO: 348 - DNA, 100 bp, Artificial Sequence; identical to SEQ ID NO: 325
  Description of Artificial Sequence: Synthetic polynucleotide

SEQ ID NO: 349 - DNA, 100 bp, Artificial Sequence
  Description of Artificial Sequence: Synthetic polynucleotide
atgggtatggcctctaaaaacatggccccagcagcttcagtcccttactcgtcgatggtcagcacagccttatgcacggcctggaggggagagaagcaga

SEQ ID NO: 350 - DNA, 60 bp, Artificial Sequence
  Description of Artificial Sequence: Synthetic oligonucleotide
catggccccagcagcttcagtcccttactcgtcgatggtcagcacagccttatgcacggc

SEQ ID NO: 351 - DNA, 65 bp, Artificial Sequence
  Description of Artificial Sequence: Synthetic oligonucleotide
aacatggccccagcagcttcagtcccttactcgtcgatggtcagcacagccttatgcacggcctg

SEQ ID NO: 352 - DNA, 70 bp, Artificial Sequence
  Description of Artificial Sequence: Synthetic oligonucleotide
aaaaacatggccccagcagcttcagtcccttactcgtcgatggtcagcacagccttatgcacggcctgga

SEQ ID NO: 353 - DNA, 75 bp, Artificial Sequence
  Description of Artificial Sequence: Synthetic oligonucleotide
tctaaaaacatggccccagcagcttcagtcccttactcgtcgatggtcagcacagccttatgcacggcctggagg

SEQ ID NO: 354 - DNA, 80 bp, Artificial Sequence
  Description of Artificial Sequence: Synthetic oligonucleotide
cctctaaaaacatggccccagcagcttcagtcccttactcgtcgatggtcagcacagccttatgcacggcctggagggga

SEQ ID NO: 355 - DNA, 85 bp, Artificial Sequence
  Description of Artificial Sequence: Synthetic oligonucleotide
ggcctctaaaaacatggccccagcagcttcagtcccttactcgtcgatggtcagcacagccttatgcacggcctggaggggagag

SEQ ID NO: 356 - DNA, 95 bp, Artificial Sequence
  Description of Artificial Sequence: Synthetic oligonucleotide
atgggtatggcctctaaaaacatggccccagcagcttcagtcccttactcgtcgatggtcagcacagccttatgcacggcctggaggggagagaa

SEQ ID NO: 357 - skipped

SEQ ID NO: 358 - DNA, 100 bp, Artificial Sequence
  Description of Artificial Sequence: Synthetic polynucleotide
tgacctcgggggggatagacatgggtatggcctctaaaaacatggccccagcagcttcagtcccttactcgtcgatggtcagcacagccttatgcacggc

SEQ ID NO: 359 - DNA, 95 bp, Artificial Sequence
  Description of Artificial Sequence: Synthetic oligonucleotide
tatggcctctaaaaacatggccccagcagcttcagtcccttactcgtcgatggtcagcacagccaaatgcacggcctggaggggagagaagcaga

SEQ ID NO: 360 - DNA, 98 bp, Artificial Sequence
  Description of Artificial Sequence: Synthetic oligonucleotide
tatggcctctaaaaacatggccccagcagcttcagtcccttactcgtcgatggtcagcacagccccagtatgcacggcctggaggggagagaagcaga

SEQ ID NO: 361 - DNA, 99 bp, Artificial Sequence
  Description of Artificial Sequence: Synthetic oligonucleotide
tatggcctctaaaaacatggccccagcagcttcagtcccttactcgtcgatggtcagcacagccagtcgtatgcacggcctggaggggagagaagcaga

SEQ ID NO: 362 - DNA, 95 bp, Artificial Sequence; identical to SEQ ID NO: 356
  Description of Artificial Sequence: Synthetic oligonucleotide

SEQ ID NO: 363 - skipped

SEQ ID NO: 364 - DNA, 99 bp, Artificial Sequence
  Description of Artificial Sequence: Synthetic oligonucleotide
atgggtatggcctctaaaaacatggccccagcagcttcagtcccttactcgtcgatggtcagcacagccagtcgtatgcacggcctggaggggagagaa

SEQ ID NO: 365 - DNA, 103 bp, Artificial Sequence
  Description of Artificial Sequence: Synthetic polynucleotide
acatgggtatggcctctaaaaacatggccccagcagcttcagtcccttactcgtcgatggtcagcacagccagtcgtatgcacggcctggaggggagagaagc

SEQ ID NO: 366 - DNA, 107 bp, Artificial Sequence
  Description of Artificial Sequence: Synthetic polynucleotide
agacatgggtatggcctctaaaaacatggccccagcagcttcagtcccttactcgtcgatggtcagcacagccagtcgtatgcacggcctggaggggagagaagcag

SEQ ID NO: 367 - DNA, 111 bp, Artificial Sequence
  Description of Artificial Sequence: Synthetic polynucleotide
atagacatgggtatggcctctaaaaacatggccccagcagcttcagtcccttactcgtcgatggtcagcacagccagtcgtatgcacggcctggaggggagagaagcagag

SEQ ID NO: 368 - DNA, 115 bp, Artificial Sequence
  Description of Artificial Sequence: Synthetic polynucleotide
ggatagacatgggtatggcctctaaaaacatggccccagcagcttcagtcccttactcgtcgatggtcagcacagccagtcgtatgcacggcctggaggggagagaagcagagac

SEQ ID NO: 369 - DNA, 119 bp, Artificial Sequence
  Description of Artificial Sequence: Synthetic polynucleotide
ggggatagacatgggtatggcctctaaaaacatggccccagcagcttcagtcccttactcgtcgatggtcagcacagccagtcgtatgcacggcctggaggggagagaagcagagacac

SEQ ID NO: 370 - DNA, 123 bp, Artificial Sequence
  Description of Artificial Sequence: Synthetic polynucleotide
ggggggatagacatgggtatggcctctaaaaacatggccccagcagcttcagtcccttactcgtcgatggtcagcacagccagtcgtatgcacggcctggaggggagagaagcagagacacgt

SEQ ID NO: 371 - DNA, 127 bp, Artificial Sequence
  Description of Artificial Sequence: Synthetic polynucleotide
cgggggggatagacatgggtatggcctctaaaaacatggccccagcagcttcagtcccttactcgtcgatggtcagcacagccagtcgtatgcacggcctggaggggagagaagcagagacacgttg

SEQ ID NO: 372 - skipped

SEQ ID NO: 373 - DNA, 111 bp, Artificial Sequence; identical to SEQ ID NO: 367
  Description of Artificial Sequence: Synthetic polynucleotide

SEQ ID NO: 374 - DNA, 114 bp, Artificial Sequence
  Description of Artificial Sequence: Synthetic polynucleotide
atagacatgggtatggcctctccacaaaacatggccccagcagcttcagtcccttactcatcgatggtcagcacagccagtcgtatgcacggcctggaggggagagaagcagag

SEQ ID NO: 375 - DNA, 115 bp, Artificial Sequence; identical to SEQ ID NO: 368
  Description of Artificial Sequence: Synthetic polynucleotide

SEQ ID NO: 376 - DNA, 118 bp, Artificial Sequence
  Description of Artificial Sequence: Synthetic polynucleotide
ggatagacatgggtatggcctctccacaaaacatggccccagcagcttcagtcccttactcatcgatggtcagcacagccagtcgtatgcacggcctggaggggagagaagcagagac

SEQ ID NO: 377 - DNA, 123 bp, Artificial Sequence; identical to SEQ ID NO: 370
  Description of Artificial Sequence: Synthetic polynucleotide

SEQ ID NO: 378 - DNA, 126 bp, Artificial Sequence
  Description of Artificial Sequence: Synthetic polynucleotide
ggggggatagacatgggtatggcctctccacaaaacatggccccagcagcttcagtcccttactcatcgatggtcagcacagccagtcgtatgcacggcctggaggggagagaagcagagacacgt

SEQ ID NO: 379 - skipped

SEQ ID NO: 380 - DNA, 100 bp, Artificial Sequence
  Description of Artificial Sequence: Synthetic polynucleotide
tgataaaaggcgtacataagtcttgtgtctactgtacagaagactgccgccagctggatttcccaattctgagtaacactctgcaatccaaacagggttc

SEQ ID NO: 381 - DNA, 131 bp, Artificial Sequence
  Description of Artificial Sequence: Synthetic polynucleotide
ccccagcagcttcagtcccttactcgtcgatggtcagcacagccttatgcacggcccagtgtctcctctgtgaccccggagaggtcagccccaccagtgtcggacagtttgggtaaatgtaagctggcaga

SEQ ID NO: 382 - DNA, 131 bp, Artificial Sequence
  Description of Artificial Sequence: Synthetic polynucleotide
ccccagcagcttcagtcccttactcgtcgatggtcagcacagccttatgcacggcccagtgtctcctctgtgaccccggagaggtcagccccaccagtgtcgacccaggacgctcttcagatcataggttc

SEQ ID NO: 383 - DNA, 131 bp, Artificial Sequence
  Description of Artificial Sequence: Synthetic polynucleotide
ccccagcagcttcagtcccttactcgtcgatggtcagcacagccttatgcacggcccagtgtcgacccaggacgctcttcagatcataggttcccagtgtcggacagtttgggtaaatgtaagctggcaga

SEQ ID NO: 384 - DNA, 171 bp, Artificial Sequence
  Description of Artificial Sequence: Synthetic polynucleotide
tatggcctctaaaaacatggccccagcagcttcagtcccttactcgtcgatggtcagcacagccttatgcacggcctggaggggagagaagcagaccagtgtctcctctgtgaccccggagaggtcagccccaccagtgtcggacagtttgggtaaatgtaagctggcaga

SEQ ID NO: 385 - DNA, 171 bp, Artificial Sequence
  Description of Artificial Sequence: Synthetic polynucleotide
tatggcctctaaaaacatggccccagcagcttcagtcccttactcgtcgatggtcagcacagccttatgcacggcctggaggggagagaagcagaccagtgtctcctctgtgaccccggagaggtcagccccaccagtgtcgacccaggacgctcttcagatcataggttc

SEQ ID NO: 386 - DNA, 171 bp, Artificial Sequence
  Description of Artificial Sequence: Synthetic polynucleotide
tatggcctctaaaaacatggccccagcagcttcagtcccttactcgtcgatggtcagcacagccttatgcacggcctggaggggagagaagcagaccagtgtcgacccaggacgctcttcagatcataggttcccagtgtcggacagtttgggtaaatgtaagctggcaga

SEQ ID NO: 387 - DNA, 95 bp, Artificial Sequence; identical to SEQ ID NO: 356
  Description of Artificial Sequence: Synthetic oligonucleotide

SEQ ID NO: 388 - DNA, 100 bp, Artificial Sequence; identical to SEQ ID NO: 380
  Description of Artificial Sequence: Synthetic polynucleotide

SEQ ID NO: 389 - DNA, 32 bp, Artificial Sequence
  Description of Artificial Sequence: Synthetic oligonucleotide
taggctttctggctttttaccggaaagcccct

SEQ ID NO: 390 - DNA, 11 bp, Artificial Sequence
  Description of Artificial Sequence: Synthetic oligonucleotide
aatttttggag

SEQ ID NO: 391 - DNA, 100 bp, Artificial Sequence
  Description of Artificial Sequence: Synthetic polynucleotide
atgggtatggcctctaaaaacatggccccagcagcttcagtccctttctcgtcgatggtcagcacagccttatgcacggccttggagagcttcaggggtg

SEQ ID NO: 392 - skipped

SEQ ID NO: 393 - DNA, 132 bp, Artificial Sequence
  Description of Artificial Sequence: Synthetic polynucleotide
atatgctaatacgactcactataggtgccctctgatgtttttatccccattcggttacagtaatgagcaaatgagcaaatgccgagtcagcaagatttgctggttgggcagcattgggatacagtgtgaaaa

SEQ ID NO: 394 - DNA, 31 bp, Artificial Sequence
  Description of Artificial Sequence: Synthetic oligonucleotide
caggttttctgacttcggtcggaaaacccct

SEQ ID NO: 395 - DNA, 145 bp, Artificial Sequence
  Description of Artificial Sequence: Synthetic polynucleotide
atgggtatggcctctaaaaacatggccccagcagcttcagtccctttctcgtcgatggtccaccctatgatattgttgtaaatcgtataacaatatgataaggtgagcacagccttatgcacggccttggagagcttcaggggtg

SEQ ID NO: 396 - DNA, 45 bp, Artificial Sequence
  Description of Artificial Sequence: Synthetic oligonucleotide
attctaaagcaggacggggtggtgccgtagtgtcgtatctttgca

SEQ ID NO: 397 - DNA, 45 bp, Artificial Sequence
  Description of Artificial Sequence: Synthetic oligonucleotide
attctaaagcaggactgggtggtgccgtagtgtcgtatctttgca

SEQ ID NO: 398 - DNA, 45 bp, Artificial Sequence
  Description of Artificial Sequence: Synthetic oligonucleotide
attctaaagcaggaccgggtggtgccgtagtgtcgtatctttgca

SEQ ID NO: 399 - DNA, 45 bp, Artificial Sequence
  Description of Artificial Sequence: Synthetic oligonucleotide
attctagagcaggactgggtggtgccgtagtgtcgtatctttgca

SEQ ID NO: 400 - DNA, 45 bp, Artificial Sequence
  Description of Artificial Sequence: Synthetic oligonucleotide
attctacagcaggactgggtggtgccgtagtgtcgtatctttgca

SEQ ID NO: 401 - DNA, 45 bp, Artificial Sequence
  Description of Artificial Sequence: Synthetic oligonucleotide
attctaaagcatgactgggtggtgccgtagtcagcaatctttgca

SEQ ID NO: 402 - DNA, 45 bp, Artificial Sequence
  Description of Artificial Sequence: Synthetic oligonucleotide
attctaaagcaggactgggtggtgccgtagtcagcaatctttgca

SEQ ID NO: 403 - DNA, 45 bp, Artificial Sequence
  Description of Artificial Sequence: Synthetic oligonucleotide
attctaaagcagtactgggtggtgccgtagtcagcaatctttgca

SEQ ID NO: 404 - DNA, 45 bp, Artificial Sequence
  Description of Artificial Sequence: Synthetic oligonucleotide
attctagagcatgactgggtggtgccgtagtcagcaatctttgca

SEQ ID NO: 405 - DNA, 45 bp, Artificial Sequence
  Description of Artificial Sequence: Synthetic oligonucleotide
attctagagcaggacggggtggtgccgtagtcagcaatctttgca

SEQ ID NO: 406 - protein, 6 aa, Artificial Sequence
  Description of Artificial Sequence: Synthetic peptide
  MOD_RES (2)..(2): Any amino acid
KXKEGV

SEQ ID NO: 407 - protein, 9 aa, Artificial Sequence
  Description of Artificial Sequence: Synthetic peptide
VGGAVVTGV

SEQ ID NO: 408 - protein, 6 aa, Artificial Sequence
  Description of Artificial Sequence: Synthetic peptide
DPDNEA

SEQ ID NO: 409 - DNA, 56 bp, Drosophila sp.
taacagcgacgacgccaaacgccggcatgtcggctctttgtgcgtgatcgctggtg

SEQ ID NO: 410 - RNA, 29 nt, Drosophila sp.
ugucggcucuuugugcgugaucgcugguc

SEQ ID NO: 411 - RNA, 27 nt, Drosophila sp.
uaacagcgacgacgccaaacgccggca

SEQ ID NO: 412 - RNA, 27 nt, Artificial Sequence
  Description of Artificial Sequence: Synthetic oligonucleotide
uggacaggcugugugucgaaguucgcc

SEQ ID NO: 413 - RNA, 27 nt, Artificial Sequence
  Description of Artificial Sequence: Synthetic oligonucleotide
ggcgaacuucgacacacagccugucca

SEQ ID NO: 414 - RNA, 25 nt, Artificial Sequence
  Description of Artificial Sequence: Synthetic oligonucleotide
ugagaacucggaccacagccucccg

SEQ ID NO: 415 - RNA, 25 nt, Artificial Sequence
  Description of Artificial Sequence: Synthetic oligonucleotide
ugagaacucggacaccagccucccg

SEQ ID NO: 416 - RNA, 27 nt, Artificial Sequence
  Description of Artificial Sequence: Synthetic oligonucleotide
ggcgaacuccgacacacagccugucca

SEQ ID NO: 417 - RNA, 27 nt, Artificial Sequence
  Description of Artificial Sequence: Synthetic oligonucleotide
ggcgaacucggacacacagccugucca

SEQ ID NO: 418 - RNA, 25 nt, Artificial Sequence
  Description of Artificial Sequence: Synthetic oligonucleotide
ggagaacucggaccacagccuccca

SEQ ID NO: 419 - RNA, 26 nt, Artificial Sequence
  Description of Artificial Sequence: Synthetic oligonucleotide
ugcgaacucggaccacagccuguccg

SEQ ID NO: 420 - RNA, 26 nt, Artificial Sequence
  Description of Artificial Sequence: Synthetic oligonucleotide
ggcgaacucgacacacagccugucca

SEQ ID NO: 421 - RNA, 25 nt, Artificial Sequence
  Description of Artificial Sequence: Synthetic oligonucleotide
ggcgaacucacacacagccugucca

SEQ ID NO: 422 - DNA, 39 bp, Artificial Sequence
  Description of Artificial Sequence: Synthetic oligonucleotide
gtggacaggctgtgtgtcgaagttcgccctggagagtgc

SEQ ID NO: 423 - DNA, 39 bp, Artificial Sequence
  Description of Artificial Sequence: Synthetic oligonucleotide
gcactctccagtgagaactcggaccacagcctcccgctg

SEQ ID NO: 424 - DNA, 40 bp, Artificial Sequence
  Description of Artificial Sequence: Synthetic oligonucleotide
gtggacaggctgtgtgtcgaagttcgccctggagagtgct

SEQ ID NO: 425 - DNA, 40 bp, Artificial Sequence
  Description of Artificial Sequence: Synthetic oligonucleotide
agcactctccagtgagaactcggacaccagcctcccgctg

SEQ ID NO: 426 - DNA, 39 bp, Artificial Sequence
  Description of Artificial Sequence: Synthetic oligonucleotide
gcactctccagggcgaactcacacacagcctgtccactg

SEQ ID NO: 427 - DNA, 41 bp, Artificial Sequence
  Description of Artificial Sequence: Synthetic oligonucleotide
agcactctccagggcgaactcgacacacagcctgtccactg

SEQ ID NO: 428 - DNA, 40 bp, Artificial Sequence
  Description of Artificial Sequence: Synthetic oligonucleotide
gcactctccagggcgaactccgacacacagcctgtccact

SEQ ID NO: 429 - DNA, 36 bp, Artificial Sequence
  Description of Artificial Sequence: Synthetic oligonucleotide
gtggacaggctgtgtgtcgaagttcgccctggagag

SEQ ID NO: 430 - DNA, 36 bp, Artificial Sequence
  Description of Artificial Sequence: Synthetic oligonucleotide
ctctccagggcgaactcggacacacagcctgtccac

SEQ ID NO: 431 - DNA, 48 bp, Artificial Sequence
  Description of Artificial Sequence: Synthetic oligonucleotide
cagcagtggacaggctgtgtgtcgaagttcgccctggagagtgctttg

SEQ ID NO: 432 - DNA, 48 bp, Artificial Sequence
  Description of Artificial Sequence: Synthetic oligonucleotide
caaagcactctccagggcgaactccgacacacagcctgtccactgctg

SEQ ID NO: 433 - DNA, 47 bp, Artificial Sequence
  Description of Artificial Sequence: Synthetic oligonucleotide
cagcagtggacagcctgtgtgtcgaagttcgccctggagagtgcttt

SEQ ID NO: 434 - DNA, 47 bp, Artificial Sequence
  Description of Artificial Sequence: Synthetic oligonucleotide
aaagcactctccagggcgaactcggacacacagcctgtccactgctg

SEQ ID NO: 435 - DNA, 45 bp, Artificial Sequence
  Description of Artificial Sequence: Synthetic oligonucleotide
cagcagtggacaggctgtgtgtcgaagttcgccctggagagtgct

SEQ ID NO: 436 - DNA, 43 bp, Artificial Sequence
  Description of Artificial Sequence: Synthetic oligonucleotide
agcactctccagggcgaactcacacacagcctgtccactgctg

SEQ ID NO: 437 - DNA, 47 bp, Artificial Sequence
  Description of Artificial Sequence: Synthetic oligonucleotide
caaagcactctccagggcgaactcgacacacagcctgtccactgctg

SEQ ID NO: 438 - DNA, 52 bp, Artificial Sequence
  Description of Artificial Sequence: Synthetic oligonucleotide
cagcagtggacaggctgtgtgtcgaagttcgccctggagagtgctttggcct

SEQ ID NO: 439 - DNA, 50 bp, Artificial Sequence
  Description of Artificial Sequence: Synthetic oligonucleotide
aggccaaagcactctccagtgagaactcggaccacagcctcccgctgctg

SEQ ID NO: 440 - DNA, 56 bp, Artificial Sequence
  Description of Artificial Sequence: Synthetic oligonucleotide
ccagcagtggacaggctgtgtgtcgaagttcgccctggagagtgctttggcctcct

SEQ ID NO: 441 - DNA, 53 bp, Artificial Sequence
  Description of Artificial Sequence: Synthetic oligonucleotide
aggaggccaaagcactctccagtgagaactcggacaccagcctcccactgctg

SEQ ID NO: 442 - DNA, 51 bp, Artificial Sequence
  Description of Artificial Sequence: Synthetic oligonucleotide
ccagcagtggacaggctgtgtgtcgaagttcgccctggagagtgctttggc

SEQ ID NO: 443 - DNA, 48 bp, Artificial Sequence
  Description of Artificial Sequence: Synthetic oligonucleotide
gccaaagcactctccagggagaactcggaccacagcctcccactgctg

SEQ ID NO: 444 - DNA, 49 bp, Artificial Sequence
  Description of Artificial Sequence: Synthetic oligonucleotide
ccagcagtggacaggctgtgtgtcgaagttccccctggagagtgctttg

SEQ ID NO: 445 - DNA, 46 bp, Artificial Sequence
  Description of Artificial Sequence: Synthetic oligonucleotide
caaagcactctccagggagaactcggacaccagcctcccactgctg

SEQ ID NO: 446 - DNA, 46 bp, Artificial Sequence
  Description of Artificial Sequence: Synthetic oligonucleotide
gcagtggacaggctgtgtgtcgaagttcgccctggagagtgctttg

SEQ ID NO: 447 - DNA, 46 bp, Artificial Sequence
  Description of Artificial Sequence: Synthetic oligonucleotide
caaagcactctccagggcgaactccgacacacagcctgtccactgc

SEQ ID NO: 448 - DNA, 53 bp, Artificial Sequence
  Description of Artificial Sequence: Synthetic oligonucleotide
gcccagcagtggacaggctgtgtgtcgaagttcgccctggagagtgctttggc

SEQ ID NO: 449 - DNA, 53 bp, Artificial Sequence
  Description of Artificial Sequence: Synthetic oligonucleotide
gccaaagcactctccagggcgaactcggacacacagcctgtccactgctgggc

SEQ ID NO: 450 - DNA, 43 bp, Artificial Sequence
  Description of Artificial Sequence: Synthetic oligonucleotide
cagtggacaggctgtgtgtcgaagttcgccctggagagtgctt

SEQ ID NO: 451 - DNA, 41 bp, Artificial Sequence
  Description of Artificial Sequence: Synthetic oligonucleotide
aagcactctccagggcgaactcacacacagcctgtccactg

SEQ ID NO: 452 - DNA, 46 bp, Artificial Sequence
  Description of Artificial Sequence: Synthetic oligonucleotide
agcagtggacaggctgtctgtcgaagttcgccctggagagtgcttt

SEQ ID NO: 453 - DNA, 45 bp, Artificial Sequence
  Description of Artificial Sequence: Synthetic oligonucleotide
aaagcactctccagggcgaactcgacacacagcctgtccactgct

SEQ ID NO: 454 - DNA, 59 bp, Artificial Sequence
  Description of Artificial Sequence: Synthetic oligonucleotide
cctccagcccagcagtggacaggctgtgtgtcgaagttcgccctggagagtgctttggc

SEQ ID NO: 455 - DNA, 57 bp, Artificial Sequence
  Description of Artificial Sequence: Synthetic oligonucleotide
gccaaagcactctccagtgagaactcggaccacagcctcccgctgctgggctggagg

SEQ ID NO: 456 - DNA, 60 bp, Artificial Sequence
  Description of Artificial Sequence: Synthetic oligonucleotide
ctccagcccagcagtggacaggctgtgtgtcgaagttcgccctggagagtgctttggcct

SEQ ID NO: 457 - DNA, 58 bp, Artificial Sequence
  Description of Artificial Sequence: Synthetic oligonucleotide
aggccaaagcactctccagtgagaactcggacaccagcctcccactgctgggctggag

SEQ ID NO: 458 - DNA, 54 bp, Artificial Sequence
  Description of Artificial Sequence: Synthetic oligonucleotide
tccagcccagcagtggacaggctgtgtgtcgaagttcgccctggagagtgcttt

SEQ ID NO: 459 - DNA, 52 bp, Artificial Sequence
  Description of Artificial Sequence: Synthetic oligonucleotide
aaagcactctccagggagaactcggaccacagcctcccactgctgggctgga

SEQ ID NO: 460 - DNA, 51 bp, Artificial Sequence
  Description of Artificial Sequence: Synthetic oligonucleotide
tccagcccagcagtggacaggctgtgtgtcgaagttcgccctggagagtgc

SEQ ID NO: 461 - DNA, 49 bp, Artificial Sequence
  Description of Artificial Sequence: Synthetic oligonucleotide
gcactctccagggagaactcggacaccagcctcccactgctgggctgga

SEQ ID NO: 462 - DNA, 57 bp, Artificial Sequence
  Description of Artificial Sequence: Synthetic oligonucleotide
cccagcagtggacaggctgtgtgtcgaagttcgccctggagagtgctttggcctcct

SEQ ID NO: 463 - DNA, 56 bp, Artificial Sequence
  Description of Artificial Sequence: Synthetic oligonucleotide
aggaggccaaagcactctccagtgcgaactcggaccacagcctgtccgctgctggg

SEQ ID NO: 464 - DNA, 52 bp, Artificial Sequence
  Description of Artificial Sequence: Synthetic oligonucleotide
gcagtggacaggctgtgtgtcgaagttcgccctggagagtgctttggcctcc

SEQ ID NO: 465 - DNA, 51 bp, Artificial Sequence
  Description of Artificial Sequence: Synthetic oligonucleotide
ggaggccaaagcactctccagtgcgaactcggacaccagcctgtccgctgc

SEQ ID NO: 466 - RNA, 200 nt, Artificial Sequence
  Description of Artificial Sequence: Synthetic polynucleotide
ugguccaagacccaacuguuuauaguucugccuccucuagagacuucacuccuaccuacgucuuaaggcuguacugaguccuauacuucaaguaguaguuaacuacuacuugaaguauaggacucaguacagccuuaagacguagguagaagugaagucucuagaggaggcagaacuauaaacaguugggucuuggacca

SEQ ID NO: 467 - DNA, 120 bp, Artificial Sequence
  Description of Artificial Sequence: Synthetic polynucleotide
cagcccagcagtggacaggctgtgtgtcgaagttcgccctggagaggtgggtactctgcacaaagcactctccagtgagaactcggacaccagcctcccactgctgggctggaggtgcct

SEQ ID NO: 468 - DNA, 39 bp, Drosophila sp.
tggacaggctgtgtgtcgaagttcgccctggagagtgct

SEQ ID NO: 469 - DNA, 36 bp, Drosophila sp.
tggacaggctgtgtgtcgaagttcgccctggagagt

SEQ ID NO: 470 - RNA, 29 nt, Artificial Sequence
  Description of Artificial Sequence: Synthetic oligonucleotide
ugucggcucuuugugcgugaucgcuggug

SEQ ID NO: 471 - DNA, 180 bp, Artificial Sequence
  Description of Artificial Sequence: Synthetic polynucleotide
tcctctagaccttcacttgcacctacttcttaaggctgtactgagtcctatacttcaagtagtagtttttaaccacaagaaacgtcttctgaagacgtttcttgtggttaaaaactactacttgaagtataggactcagtacagccttaagacgtaggtagaagtgaagtctctagagga

SEQ ID NO: 472 - DNA, 90 bp, Artificial Sequence
  Description of Artificial Sequence: Synthetic oligonucleotide
tcttctgcaaagaacaccaatttttgatgatgaacttcatatcctgagtcatgtcggaattcttcatccacgttcacttccagatctcct

SEQ ID NO: 473 - DNA, 120 bp, Artificial Sequence
  Description of Artificial Sequence: Synthetic polynucleotide
tagttctgcctcctctagaccttcacttgcacctacttcttaaggctgtactgagtcctaaggactcagtacagccttaagacgtaggtagaagtgaagtctctagaggaggcagaacta

SEQ ID NO: 474 - DNA, 60 bp, Artificial Sequence
  Description of Artificial Sequence: Synthetic oligonucleotide
atcctgagtcatgtcggaattcttcatccacgttcacttccagatctcctccgtcttgat

SEQ ID NO: 475 - DNA, 120 bp, Artificial Sequence
  Description of Artificial Sequence: Synthetic polynucleotide
tcctctagaccttcacttgcacctacttcttaaggctgtactgagtcctatacttcaagtcttgaagtataggactcagtacagccttaagacgtaggtagaagtgaagtctctagagga

SEQ ID NO: 476 - DNA, 60 bp, Artificial Sequence
  Description of Artificial Sequence: Synthetic oligonucleotide
tgaacttcatatcctgagtcatgtcggaattcttcatccacgttcacttccagatctcct

SEQ ID NO: 477 - DNA, 180 bp, Artificial Sequence
  Description of Artificial Sequence: Synthetic polynucleotide
agacccaactgtttatagttctgcctcctctagaccttcacttgcacctacttcttaaggctgtactgagtcctatacttcaagtagtagtactacttgaagtataggactcagtacagccttaagacgtaggtagaagtgaagtctctagaggaggcagaactataaacagttgggtct

SEQ ID NO: 478 - DNA, 90 bp, Artificial Sequence
  Description of Artificial Sequence: Synthetic oligonucleotide
gatgatgaacttcatatcctgagtcatgtcggaattcttcatccacgttcacttccagatctcctccgtcttgatatttgtcaacccaga

SEQ ID NO: 479 - DNA, 45 bp, Artificial Sequence
  Description of Artificial Sequence: Synthetic oligonucleotide
tcattgcaaagattgctgactacagcattgctcagtactgctgta

SEQ ID NO: 480 - DNA, 45 bp, Artificial Sequence
  Description of Artificial Sequence: Synthetic oligonucleotide
agcagtggacaggctgtgtgtcgaagttcgccctggagaggtggg

SEQ ID NO: 481 - DNA, 46 bp, Artificial Sequence
  Description of Artificial Sequence: Synthetic oligonucleotide
gagcagtggacaggctgtgtgtcgaagttcgccctggagaggtggg

SEQ ID NO: 482 - DNA, 48 bp, Artificial Sequence; identical to SEQ ID NO: 431
  Description of Artificial Sequence: Synthetic oligonucleotide

SEQ ID NO: 483 - DNA, 44 bp, Artificial Sequence
  Description of Artificial Sequence: Synthetic oligonucleotide
taaggctgtgctgaccatcgacaagaaagggactgaagctgctg

SEQ ID NO: 484 - DNA, 28 bp, Artificial Sequence
  Description of Artificial Sequence: Synthetic oligonucleotide
accatcgacaagaaagggactgaagctg

SEQ ID NO: 485 - RNA, 200 nt, Artificial Sequence
  Description of Artificial Sequence: Synthetic polynucleotide
aaaagugugacauaggguuacgacgguaguaacguuucuuugcugugaugccgugacgaggcauuacgacaucuuaccccuauuuuuguagucucccgugcacgggagacuacaaaaauagggguaagaugucgucaugacucguuacgacaucagucguuagaaacguuacuaccgucguaacccuaugucacacuuuu

SEQ ID NO: 486 - RNA, 200 nt, Artificial Sequence
  Description of Artificial Sequence: Synthetic polynucleotide
aacguuucuuugcugugaugccgugacgaggcauuacgacaucuuaccccuauuuuuguagucucccguguggucccaaagcacguggacuucaacggucgaccguugaaguccacgugcuuugggaccacacgggagacuacaaaaauagggguaagaugucgucaugacucguuacgacaucagucguuagaaacguu

SEQ ID NO: 487 - DNA, 32 bp, Artificial Sequence
  Description of Artificial Sequence: Synthetic oligonucleotide
aggcacctccagcccagcagtggacaggctgt

SEQ ID NO: 488 - DNA, 35 bp, Artificial Sequence
  Description of Artificial Sequence: Synthetic oligonucleotide
gccctggagagtgctttggcctcctgggagtgaat

SEQ ID NO: 489 - DNA, 199 bp, Artificial Sequence
  Description of Artificial Sequence: Synthetic polynucleotide
cgtcacctgtagacaacaggctcaagcagggcctctcacgaaaccggaggaccctcacttaccacggccgttttgttggtgtaagttctacgagtgacccgggtcactcgtagaacttacaccaacaaaacggccgtggtaagtgagggtcctccggtttcgtgagaggtcccgcttgaagctgttgtcggacaggtga

SEQ ID NO: 490 - DNA, 200 bp, Artificial Sequence
  Description of Artificial Sequence: Synthetic polynucleotide
tcacctgtcggacacacaggctcaagcagggcctctcacgaaaccggaggaccctcacttaccacggccgttttgttggtgtaagttctacgagtgacccgggtcactcgtagaacttacaccaacaaaacggccgtggtaagtgagggtcctccggtttcgtgagaggtcccgcttgaagctgtgtgtcggacaggtga

SEQ ID NO: 491 - DNA, 200 bp, Artificial Sequence
  Description of Artificial Sequence: Synthetic polynucleotide
tcacctgtccgacacacaggttcaagcgggacttctcacgaaaccggaggaccctcacttaccacggccgttttgttggtgtaagttctacgagtgacccgggtcactcgtagaacttacaccaacaaaacggccgtggtaagtgagggtcctccggtttcgtgagaggtcccgcttgaagctgtgtgtcggacaggtga

SEQ ID NO: 492 - DNA, 200 bp, Artificial Sequence
  Description of Artificial Sequence: Synthetic polynucleotide
tcacctgtccgacacacaggttctagcgggacttctcacgaaaccggaggaccctcacttaccacggccgttttgttggtgtaagttctacgagtgacccgggtcactcgtagaacttacaccaacaaaacggccgtggtaagtgagggtcctccggtttcgtgagaggtcccgcttgaagctgtgtgtcggacaggtga

SEQ ID NO: 493 - DNA, 200 bp, Artificial Sequence
  Description of Artificial Sequence: Synthetic polynucleotide
tcacctgtccgacatacaggttcaagcgggacctcgcacgaaaccggaggaccctcacttaccacggccgttttgttggtgtaagttctacgagtgacccgggtcactcgtagaacttacaccaacaaaacggccgtggtaagtgagggtcctccggtttcgtgagaggtcccgcttgaagctgtgtgtcggacaggtga

SEQ ID NO: 494 - RNA, 200 nt, Artificial Sequence
  Description of Artificial Sequence: Synthetic polynucleotide
ucaccuguccgacauacugguucaagcgggaccucucacgaaaccggaggacccucacuuaccacggccguuuuguugguguaaguucuacgagugacccgggucacucguagaacuuacaccaacaaaacggccgugguaagugaggguccuccgguuucgugagaggucccgcuugaagcugugugucggacagguga

SEQ ID NO: 495 - RNA, 101 nt, Artificial Sequence
  Description of Artificial Sequence: Synthetic polynucleotide
agcaguggacaggcugugugucgaaguucgcccuggagaggugggauaugcuaaaugucccaccucuccgggacgaacucggacaacaguuguccacugcu

SEQ ID NO: 496 - RNA, 101 nt, Artificial Sequence
  Description of Artificial Sequence: Synthetic polynucleotide
agcaguggacaggcugugugucgaaguucgcccuggagaggugggauaugcuaaaugucccaccucuccgggacgaacucggacaacagcuguccacugcu

SEQ ID NO: 497 - RNA, 103 nt, Artificial Sequence
  Description of Artificial Sequence: Synthetic polynucleotide
agcaguggacaggcugugugucgaaguucgcccuggagaggugggauaugcuaaaugucccaccucuccgggacgaacucggacacacaggcuguccacugcu

SEQ ID NO: 498 - RNA, 101 nt, Artificial Sequence
  Description of Artificial Sequence: Synthetic polynucleotide
agcaguggacaggcugugugucgaaguucgcccuggagaggugggauaugcuaaaugucccaccucuccaggacgaacucggacaacagauguccacugcu

SEQ ID NO: 499 - RNA, 101 nt, Artificial Sequence
  Description of Artificial Sequence: Synthetic polynucleotide
agcaguggacaggcugugugucgaaguucgcccuggagaggugggauaugcuaaaugucccaccucuccaggacgaacucggacaacagcuguccacugcu

SEQ ID NO: 500 - RNA, 103 nt, Artificial Sequence
  Description of Artificial Sequence: Synthetic polynucleotide
agcaguggacaggcugugugucgaaguucgcccuggagaggugggauaugcuaaaugucccaccucuccagggcgaacuuggacacacagccuguccacugcu

SEQ ID NO: 501 - RNA, 103 nt, Artificial Sequence
  Description of Artificial Sequence: Synthetic polynucleotide
agcaguggacaggcugugugucgaaguucgcccuggagaggugggauaugcuaaaugucccaccgcuccagggcgaauuuggacacacagccuguccacugcu

SEQ ID NO: 502 - RNA, 103 nt, Artificial Sequence
  Description of Artificial Sequence: Synthetic polynucleotide
agcaguggacaggcugugugucgaaguucgcccuggagaggugggauaugcuaaaugucccaccgcuccagggcgaacuuggucacacagccuguccacugcu

SEQ ID NO: 503 - RNA, 103 nt, Artificial Sequence
  Description of Artificial Sequence: Synthetic polynucleotide
agcaguggacaggcugugugucgaaguucgcccuggagaggugggauaugcuaaaugucccaccgcuccagggcgaauuuggucauacagccuguccacugcu

SEQ ID NO: 504 - RNA, 103 nt, Artificial Sequence
  Description of Artificial Sequence: Synthetic polynucleotide
agcaguggacaggcugugugucgaaguucgcccuggagaggugggauaugcuaaaugucccaccucuccagggcgaauuuggucauacagccuguccacugcu

SEQ ID NO: 505 - RNA, 103 nt, Artificial Sequence
  Description of Artificial Sequence: Synthetic polynucleotide
agcaguggacaggcugugugucgaaguucgcccuggagaggugggauaugcuaaaugucccaccucuccagggcgaacuuggacauacagccuguccacugcu

SEQ ID NO: 506 - RNA, 103 nt, Artificial Sequence
  Description of Artificial Sequence: Synthetic polynucleotide
agcaguggacaggcugugugucgaaguucgcccuggagaggugggauaugcuaaaugucccaccucuccagggcgaauuuggacacacagccuguccacugcu

SEQ ID NO: 507 - RNA, 103 nt, Artificial Sequence
  Description of Artificial Sequence: Synthetic polynucleotide
agcaguggacaggcugugugucgaaguucgcccuggagaggugggauaugcuaaaugucccaccgcuccagggcgaacuuggucauacagccuguccacugcu

SEQ ID NO: 508 - RNA, 103 nt, Artificial Sequence
  Description of Artificial Sequence: Synthetic polynucleotide
agcaguggacaggcugugugucgaaguucgcccuggagaggugggauaugcuaaaugucccaccucuucagggcgaucuuggacacacagccuguccacugcu

SEQ ID NO: 509 - RNA, 103 nt, Artificial Sequence
  Description of Artificial Sequence: Synthetic polynucleotide
agcaguggacaggcugugugucgaaguucgcccuggagaggugggauaugcuaaaugucccaccucuucagggcgaucuuggacacacaaccuguccacugcu

SEQ ID NO: 510 - RNA, 103 nt, Artificial Sequence
  Description of Artificial Sequence: Synthetic polynucleotide
agcaguggacaggcugugugucgaaguucgcccuggagaggugggauaugcuaaauguccgaccucuucagggcgaucuuggacacacaaccuguccacugcu

SEQ ID NO: 511 - DNA, 200 bp, Artificial Sequence
  Description of Artificial Sequence: Synthetic polynucleotide
gtggacaggctgtgtgtcgaagttcgccctggagagtgctttggcctcctgggagtgaatggtgccggcaaaacaaccacattcaagatgctcactggggccccagtgagcatcttgaatgtggttgtattgccggcaccattcactcccaggaggccaaagcactctccagtgagaactcggaccacagcctcccgctg

SEQ ID NO: 512 - RNA, 101 nt, Artificial Sequence
  Description of Artificial Sequence: Synthetic polynucleotide
agcaguggacaggcugugugucgaaguucgcccuggagaggugggauaugcuaaaugucccaccucuccggggcgaacucggacaacaguuguccacugcu

SEQ ID NO: 513 - RNA, 101 nt, Artificial Sequence
  Description of Artificial Sequence: Synthetic polynucleotide
agcaguggacaggcugugugucgaaguucgcccuggagaggugggauaugcuaaaugucccaccucuccgggacgaacucggacaacagauguccacugcu

SEQ ID NO: 514 - RNA, 101 nt, Artificial Sequence
  Description of Artificial Sequence: Synthetic polynucleotide
agcaguggacaggcugugugucgaaguucgcccuggagaggugggauaugcuaaaugucccaccucuccaggacgaacucggacaacaguuguccacugcu

SEQ ID NO: 515 - RNA, 103 nt, Artificial Sequence
  Description of Artificial Sequence: Synthetic polynucleotide
agcaguggacaggcugugugucgaaguucgcccuggagaggugggauaugcuaaaugucccaccucuccaggacgaacucggacacacaggcuguccacugcu

SEQ ID NO: 516 - RNA, 103 nt, Artificial Sequence
  Description of Artificial Sequence: Synthetic polynucleotide
agcaguggacaggcugugugucgaaguucgcccuggagaggugggauaugcuaaaugucccaccgcuccagggcgaauuuggacauacagccuguccacugcu

SEQ ID NO: 517 - RNA, 104 nt, Artificial Sequence
  Description of Artificial Sequence: Synthetic polynucleotide
agcaguggacaggcugugugucgaaguucgcccuggagaggugggauaugcuaaaugucccaccgcuccagggcgaacuuaucacauacagccuguccacugcu

SEQ ID NO: 518 - RNA, 103 nt, Artificial Sequence
  Description of Artificial Sequence: Synthetic polynucleotide
agcaguggacaggcugugugucgaaguucgcccuggagaggugggauaugcuaaaugucccaccgcuccagggcgaauuuggucacacagccuguccacugcu

SEQ ID NO: 519 - RNA, 103 nt, Artificial Sequence
  Description of Artificial Sequence: Synthetic polynucleotide
agcaguggacaggcugugugucgaaguucgcccuggagaggugggauaugcuaaaugucccaccucuccagggcgaauuuggucacacagccuguccacugcu

SEQ ID NO: 520 - RNA, 103 nt, Artificial Sequence
  Description of Artificial Sequence: Synthetic polynucleotide
agcaguggacaggcugugugucgaaguucgcccuggagaggugggauaugcuaaaugucccaccgcuccagggcgaacuuggacauacagccuguccacugcu

SEQ ID NO: 521 - RNA, 103 nt, Artificial Sequence
  Description of Artificial Sequence: Synthetic polynucleotide
agcaguggacaggcugugugucgaaguucgcccuggagaggugggauaugcuaaaugucccaccucuccagggcgaacuuggucauacagccuguccacugcu

SEQ ID NO: 522 - RNA, 103 nt, Artificial Sequence
  Description of Artificial Sequence: Synthetic polynucleotide
agcaguggacaggcugugugucgaaguucgcccuggagaggugggauaugcuaaaugucccaccucuccagggcgaauuuggacauacagccuguccacugcu

SEQ ID NO: 523 - RNA, 103 nt, Artificial Sequence
  Description of Artificial Sequence: Synthetic polynucleotide
agcaguggacaggcugugugucgaaguucgcccuggagaggugggauaugcuaaauguccgaccucuucagggcgaucuuggacacacagccuguccacugcu

SEQ ID NO: 524 - RNA, 103 nt, Artificial Sequence
  Description of Artificial Sequence: Synthetic polynucleotide
agcaguggacaggcugugugucgaaguucgcccuggagaggugggauaugcuaaauguccgaccucuccagggcgaucuuggacacacagccuguccacugcu

SEQ ID NO: 525 - DNA, 200 bp, Artificial Sequence
  Description of Artificial Sequence: Synthetic polynucleotide
gtggacaggctgtgtgtcgaagttcgccctggagagtgctttggcctcctgggagtgaatggtgccggcaaaacaaccacattcaagatgctcactggggccccagtgagcatcttgaatgtggttgttttgccggcaccattcactcccaggaggccaaagcactctcctctgagaactcggaccacagcctcccgctg

SEQ ID NO: 526 - DNA, 200 bp, Artificial Sequence
  Description of Artificial Sequence: Synthetic polynucleotide
gtggacaggctgtgtgtcgaagttcgccctggagagtgctttggcctcctgggagtgaatggtgccggcaaaacaaccacattcaagatgctcactggggccccagtgagcatcttgaatgtggttgttttgccggcaccattcagaggcaggaggccaaagcactctcctctgagaactcggaccacagcctcccgctg

SEQ ID NO: 527 - DNA, 200 bp, Artificial Sequence
  Description of Artificial Sequence: Synthetic polynucleotide
gtggacaggctgtgtgtcgaagttcgccctggagagtgctttggcctcctgggagtgaatggtgccggcaaaacaaccacattcaagatgctcactggggccccagtgagcatcttgaatgtggttgttttgcgccgaccattcagaggcaggaggccaaagcactctcctctgagaactcggaccacagcctcccgctg

SEQ ID NO: 528 - DNA, 200 bp, Artificial Sequence
  Description of Artificial Sequence: Synthetic polynucleotide
gtggacaggctgtgtgtcgaagttcgccctggagagtgctttggcctcctgggagtgaatggtgccggcaaaacaaccacattcaagatgctcactggggccccagtgagcatcttgaatgtccatgttttgcgccgaccattcagaggcaggaggccaaagcactctcctctgagaactcggaccacagcctcccgctg

SEQ ID NO: 529 - DNA, 273 bp, Artificial Sequence
  Description of Artificial Sequence: Synthetic polynucleotide
  modified_base (160)..(164): a, c, t, g, unknown or other
atagggatagggacaactccctccttggcctttgaaagtcctttcatgaatacatccacggctaatgaattcctttacaccacactgtcgtcgaatggccactcccagttctcatataatttttggagcaggttttctgacttcggtcggaaaacccctnnnnnactgggagtggccattcgacgacagtgtggtgtaaaggaattcattagccatggatgtattcatgaaaggactttcaaaggccaaggagggagttgtggctgctgctga

SEQ ID NO: 530 - DNA, 300 bp, Artificial Sequence
  Description of Artificial Sequence: Synthetic polynucleotide
  modified_base (183)..(187): a, c, t, g, unknown or other
taatagggatagggatagggacaactccctccttggcctttgaaagtcctttcatgaatacatccacggctaatgtggaatagtataacaatatgctaaatgttgttatagtatcccacgaattcctttacaccacatataatttttggagcaggttttctgacttcggtcggaaaacccctnnnnnactgggagtggccattcgacgacagtgtggtgtaaaggaattcattagccatggatgtattcatgaaaggactttcaaaggccaaggagggagttgtggctgctgctgagaaa

SEQ ID NO: 531 - DNA, 319 bp, Artificial Sequence
  Description of Artificial Sequence: Synthetic polynucleotide
  modified_base (201)..(206): a, c, t, g, unknown or other
accgataatcaactccctccttggcctttgaaagtcctttcatgaatacaccctatgatattgttgtaaatcgtataacaatatgataaggtgcatccacggctaatgaattcctttacaccacactgtcgtcgaatggccactcccagttctcatataatttttggagcaggttttctgacttcggtcggaaaacccctnnnnnnactgggagtggccattcgacgacagtgtggtgtaaaggaattcattagccatggatgtattcatgaaaggactttcaaaggccaaggagggagttgtggctgctgctgagaaa

SEQ ID NO: 532 - DNA, 255 bp, Artificial Sequence
  Description of Artificial Sequence: Synthetic polynucleotide
  modified_base (51)..(55): a, c, t, g, unknown or other
tctcatataatttttggagcaggttttctgacttcggtcggaaaacccctnnnnnactgggagtggccattcgacgacagtgtggtgtaaaggaattcattagccatggatgtattcatgaaaggactttcaaaggccaaggagggagttgtggcgggacaactccctccttggcctttgaaagtcctttcatgaatacatccatagctaatgaattcctttacaccacactgtcgtcgaatggccactcccagt

SEQ ID NO: 533 - DNA, 229 bp, Artificial Sequence
  Description of Artificial Sequence: Synthetic polynucleotide
  modified_base (124)..(129): a, c, t, g, unknown or other
taatagggatagggatagggaccctgtttggttctctcagcagcagccacaactccctccttggcctttgaaagtcctttcatgaatacatccatagctaatgaattcctttacaccacatatnnnnnntgtggtgtaaaggaattcattagccatggatgtattcatgaaaggactttcaaaggccaaggagggagttgtggctgctgctgagaaaaccaaacagggt

SEQ ID NO: 534 - DNA, 100 bp, Artificial Sequence
  Description of Artificial Sequence: Synthetic polynucleotide
  modified_base (1)..(1): a, c, t, g, unknown or other
ncaaagtgtattttatgttttccagtgtggtgtaaaggaattcattagccatggatgtattcatgaaaggactttcaaaggccaaggagggagttgtggc

SEQ ID NO: 535 - DNA, 100 bp, Artificial Sequence
  Description of Artificial Sequence: Synthetic polynucleotide
gccacaactccctccttggcctttgaaagtcctttgatgcatacatccacggctaatgaattcctttacaccacactggaaaacataaaatacactttga

SEQ ID NO: 536 - DNA, 337 bp, Artificial Sequence
  Description of Artificial Sequence: Synthetic polynucleotide
  modified_base (150)..(154): a, c, t, g, unknown or other
gccacaactccctccttggcctttgaaagtcctttgatgcatacatccacggctaatgaattcctttacaggagagtcaattatgatattatagagtttgactgaaaaatcatgagaaaccctccaaagtgtgagactcttttaagtgtnnnnnacacttaaaagagtctcacactttggagggtttctcatgatttttcagtgttttttgtttatttttccccgaaagttctcattcaaagtgtattttatgttttccagttgtggtgtaaaggaattcattagccatggatgtattcatgaaaggactttcaaaggccaaggagggagttgtggc

SEQ ID NO: 537 - DNA, 309 bp, Artificial Sequence
  Description of Artificial Sequence: Synthetic polynucleotide
  modified_base (131)..(135): a, c, t, g, unknown or other
gccacaactccctccttggcctttgaaagtcctttgatgcatacatccacggctaatgaattcctttacaccacattagccagaaggcttgaaggcaaggcgtgagggagcgcccaggacgctctcggagnnnnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggc

SEQ ID NO: 538 - DNA, 112 bp, Artificial Sequence
  Description of Artificial Sequence: Synthetic polynucleotide
  modified_base (1)..(1): a, c, t, g, unknown or other
ntgtggtgtaaaggaattcattagccatggatgtattcatgaaaggactttcaaaggccaaggagggagttgtggctgctgctgagaaaaccaaacagggtgtggcagaagc

SEQ ID NO: 539 - DNA, 111 bp, Artificial Sequence
  Description of Artificial Sequence: Synthetic polynucleotide
  modified_base (111)..(111): a, c, t, g, unknown or other
gcttctgccacaccctgtttggttttctcaaagggtatgaagaaggggagcttggcctttgaaagtcctttgatgcatacatccacggctaatgaattcctttacaccacn

SEQ ID NO: 540 - DNA, 100 bp, Artificial Sequence
  Description of Artificial Sequence: Synthetic polynucleotide
gccacaactccctccttggcctttgaaagtcctttgatgcatacatccacggctatacttttcctttacaccacactggaaaacataaaatacactttga

SEQ ID NO: 541 - DNA, 100 bp, Artificial Sequence
  Description of Artificial Sequence: Synthetic polynucleotide
gccacaactccctccttggcctttgaaagtcctttgatgcatatatttacggctaatgaattcctttacaccacactggaaaacataaaatacactttga

SEQ ID NO: 542 - DNA, 100 bp, Artificial Sequence
  Description of Artificial Sequence: Synthetic polynucleotide
gccacaactccctccttggcctttgaaagtgctttcacgaatacatccacggctaatgaattcctttacaccacactggaaaacataaaatacactttga

SEQ ID NO: 543 - DNA, 100 bp, Artificial Sequence
  Description of Artificial Sequence: Synthetic polynucleotide
gccacaactccctccttggcctttgaaagtgctttcacgaatacatccacggctatacttttcctttacaccacactggaaaacataaaatacactttga

SEQ ID NO: 544 - DNA, 99 bp, Artificial Sequence
  Description of Artificial Sequence: Synthetic oligonucleotide
  modified_base (1)..(1): a, c, t, g, unknown or other
nactgggagtggccattcgacgacagtgtggtgtaaaggaattcattagccatggatgtattcatgaaaggactttcaaaggccaaggagggagttgtg

SEQ ID NO: 545 - DNA, 98 bp, Artificial Sequence
  Description of Artificial Sequence: Synthetic oligonucleotide
caactccctccttggcctttgaaagtcctttcatgaatacatccacggctaatgaattcctttacaccacactgtcgtcgaatggccactcccagttc

SEQ ID NO: 546 - DNA, 100 bp, Artificial Sequence
  Description of Artificial Sequence: Synthetic polynucleotide
  modified_base (1)..(2): a, c, t, g, unknown or other
nnactgggagtggccattcgacgacagtgtggtgtaaaggaattcattagccatggatgtattcatgaaaggactttcaaaggccaaggagggagttgtg

SEQ ID NO: 547 - DNA, 98 bp, Artificial Sequence
  Description of Artificial Sequence: Synthetic oligonucleotide
caactccctccttggcctttgaaagtcctttcatgaatacatccatggctaatgaattcctttacaccacactgtcgtcgaatggccactcccagttc

SEQ ID NO: 548 - DNA, 174 bp, Artificial Sequence
  Description of Artificial Sequence: Synthetic polynucleotide
ctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggc

SEQ ID NO: 549 - DNA, 130 bp, Artificial Sequence
  Description of Artificial Sequence: Synthetic polynucleotide
gccacaactccctccttggcctttgaaagtcctttcatgaatacatccacggctaatgaattcctttacaccacattagccagaaggcttgaaggcaaggcgtgagggagcgcccaggacgctctcggag

SEQ ID NO: 550 - DNA, 164 bp, Artificial Sequence
  Description of Artificial Sequence: Synthetic polynucleotide
ctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagg

SEQ ID NO: 551 - DNA, 120 bp, Artificial Sequence
  Description of Artificial Sequence: Synthetic polynucleotide
cctccttggcctttgaaagtcctttcatgaatacatccacggctaatgaattcctttacaccacattagccagaaggcttgaaggcaaggcgtgagggagcgcccaggacgctctcggag

SEQ ID NO: 552 - DNA, 271 bp, Artificial Sequence
  Description of Artificial Sequence: Synthetic polynucleotide
  modified_base (111)..(115): a, c, t, g, unknown or other
ctttgaaagtcctttcatgaatacatccacggctaatgaattcctttacaccacattagccagaaggcttgaaggcaaggcgtgagggagcgcccaggacgctctcggagnnnnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggc

SEQ ID NO: 553 - DNA, 309 bp, Artificial Sequence
  Description of Artificial Sequence: Synthetic polynucleotide
  modified_base (131)..(135): a, c, t, g, unknown or other
gccacaactccctccttggcctttgaaagtcctttgatgcatacatccacggctaatgaattcctttacaccacattagccagaaggcttgaaggcaaggcgacagggagcgcccaggacgctctcggagnnnnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggc

SEQ ID NO: 554 - DNA, 330 bp, Artificial Sequence
  Description of Artificial Sequence: Synthetic polynucleotide
  modified_base (144)..(148): a, c, t, g, unknown or other
gccacaactccctccttggcctttgaaagtcctttgatgcatacatccacggctaatgaattcctttacaccacattagcaaaggcagaaggcttgaaggcaaggacaccgggagcgcccaggacgctctcggaggggccgggnnnnncccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggc

SEQ ID NO: 555 - DNA, 308 bp, Artificial Sequence
  Description of Artificial Sequence: Synthetic polynucleotide
  modified_base (130)..(134): a, c, t, g, unknown or other
gccacaactccctccttggcctttgaaagtcctttcacgaatacatccacggctaatgaattcctttacaccacattagccagaaggcttgaaggcaaggcggagggagcgcccaggacgctctcggagnnnnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggc

SEQ ID NO: 556 - DNA, 129 bp, Artificial Sequence
  Description of Artificial Sequence: Synthetic polynucleotide
  modified_base (129)..(129): a, c, t, g, unknown or other
gccacaactccctccttggcctttgaaagtcctttcacgaatacaccacggctaatgaattcctttacaccacattagccagaaggcttgaaggcaaggcggagggagcgcccaggacgctctcggagn

SEQ ID NO: 557 - DNA, 151 bp, Artificial Sequence
  Description of Artificial Sequence: Synthetic polynucleotide
  modified_base (1)..(1): a, c, t, g, unknown or other
ngagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaag

SEQ ID NO: 558 - DNA, 136 bp, Artificial Sequence
  Description of Artificial Sequence: Synthetic polynucleotide
  modified_base (136)..(136): a, c, t, g, unknown or other
ctttgaaagtcctttcatgaatacatccacggctaatgaattcctttacaccacaaggggcgaatggccactcccagttctccgctcacgagggtggaaataattaaggcgtgagggagcgcccaggacgctctcn

SEQ ID NO: 559 - DNA, 309 bp, Artificial Sequence
  Description of Artificial Sequence: Synthetic polynucleotide
  modified_base (131)..(135): a, c, t, g, unknown or other
gccacaactccctccttggcctttcgccaaaatttcatgaatacatccacggctaatgaattcctttacaccacattagccagaaggcttgaaggcaaggcgtgagggagcgcccaggacgctctcggagnnnnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggc

SEQ ID NO: 560 - DNA, 76 bp, Artificial Sequence
  Description of Artificial Sequence: Synthetic oligonucleotide
  modified_base (1)..(1): a, c, t, g, unknown or other
ntgtggtgtaaaggaattcattagccatggatgtattcatgaaaggactttcaaaggccaaggagggagttgtggc

SEQ ID NO: 561 - DNA, 76 bp, Artificial Sequence
  Description of Artificial Sequence: Synthetic oligonucleotide
  modified_base (76)..(76): a, c, t, g, unknown or other
gccacaactccctccttggcctttgaaagtcctttgatgcatacatccacggctaatgaattcctttacaccacan

SEQ ID NO: 562 - DNA, 102 bp, Artificial Sequence
  Description of Artificial Sequence: Synthetic polynucleotide
  modified_base (1)..(2): a, c, t, g, unknown or other
nnactgggagtggccattcgacgacagtgtggtgtaaaggaattcattagccatggatgtattcatgaaaggactttcaaaggccaaggagggagttgtggc

SEQ ID NO: 563 - DNA, 102 bp, Artificial Sequence
  Description of Artificial Sequence: Synthetic polynucleotide
  modified_base (101)..(102): a, c, t, g, unknown or other
gccacaactccctccttggcctttgaaagtcctttcatgccctgatccacggctaatgacttcatttacaccacactgtcgtcgaatggccactcccagtnn

SEQ ID NO: 564 - DNA, 101 bp, Artificial Sequence
  Description of Artificial Sequence: Synthetic polynucleotide
  modified_base (1)..(1): a, c, t, g, unknown or other
nactgggagtggccattcgacgacagtgtggtgtaaaggaattcattagccatggatgtattcatgaaaggactttcaaaggccaaggagggagttgtggc

SEQ ID NO: 565 - DNA, 101 bp, Artificial Sequence
  Description of Artificial Sequence: Synthetic polynucleotide
  modified_base (101)..(101): a, c, t, g, unknown or other
gccacaactccctccttggcctttgaaagtcctttcacgaatacatccacggctaatgaattcctttacaccacactgtcgtcgaatggccactcccagtn

SEQ ID NO: 566 - DNA, 100 bp, Artificial Sequence
  Description of Artificial Sequence: Synthetic polynucleotide
gccacaactccctccttggcctttgaaagtcctttcacgaatacatccacggctaatgaattcctttacaccacactggaaaacataaaatacactttga

SEQ ID NO: 567 - DNA, 100 bp, Artificial Sequence
  Description of Artificial Sequence: Synthetic polynucleotide
gccacaactccctccttggcctttgaataacggttcacgaatacatccacggctaatgaattcctttacaccacactggaaaacataaaatacactttga

SEQ ID NO: 568 - DNA, 125 bp, Artificial Sequence
  Description of Artificial Sequence: Synthetic polynucleotide
gccacaactccctccttggcctttgaataacggttcacgaatacatccacggctaatgaattcctttacaccacactggaaaacataaaatacactttgactgtcgtcgaatggccactcccagt

SEQ ID NO: 569 - DNA, 334 bp, Artificial Sequence
  Description of Artificial Sequence: Synthetic polynucleotide
  modified_base (156)..(160): a, c, t, g, unknown or other
gccacaactccctccttggcctttgaataacggttcacgaatacatccacggctaatgaattcctttacaccacactggaaaacataaaatacactttgattagccagaaggcttgaaggcaaggcgtgagggagcgcccaggacgctctcggagnnnnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggc

SEQ ID NO: 570 - DNA, 101 bp, Artificial Sequence
  Description of Artificial Sequence: Synthetic polynucleotide
  modified_base (1)..(1): a, c, t, g, unknown or other
ntcaaagtgtattttatgttttccagtgtggtgtaaaggaattcattagccatggatgtattcatgaaaggactttcaaaggccaaggagggagttgtggc

SEQ ID NO: 571 - DNA, 101 bp, Artificial Sequence
  Description of Artificial Sequence: Synthetic polynucleotide
  modified_base (101)..(101): a, c, t, g, unknown or other
gccacaactccctccttggcctttgaaagtcctttcatgccctgatccacggctaatgaatccctttaaaccacactggaaaacataaaatacactttgan

SEQ ID NO: 572 - DNA, 113 bp, Artificial Sequence
  Description of Artificial Sequence: Synthetic polynucleotide
actgggagtggccattcgacgacagtgtggtgtaaaggaattcattagccatggatgtattcatgaaaggactttcaaaggccaaggagggagttgtggctgctgctgagaaa

SEQ ID NO: 573 - DNA, 113 bp, Artificial Sequence
  Description of Artificial Sequence: Synthetic polynucleotide
tttctcagcagcagccacaactccgaggaacccctttgaaagtcctttcatgaatacatccacggctaaacttctcctttacaccacactgtcgtcgaatggccactcccagt

SEQ ID NO: 574 - DNA, 114 bp, Artificial Sequence
  Description of Artificial Sequence: Synthetic polynucleotide
  modified_base (1)..(2): a, c, t, g, unknown or other
nncaaagtgtattttatgttttccagtgtggtgtaaaggaattcattagccatggatgtattcatgaaaggactttcaaaggccaaggagggagttgtggctgctgctgagaaa

SEQ ID NO: 575 - DNA, 114 bp, Artificial Sequence
  Description of Artificial Sequence: Synthetic polynucleotide
  modified_base (114)..(114): a, c, t, g, unknown or other
tttctcagcagcagccacaactccgaggaacccctttgaaagtcctttcatgaatacatccacggctaaacttctcctttacaccacactggaaaacataaaatacactttgan

SEQ ID NO: 576 - DNA, 100 bp, Artificial Sequence
  Description of Artificial Sequence: Synthetic polynucleotide
actgggagtggccattcgacgacagtgtggtgtaaaggaattcattagccatggatgtattcatgaaaggactttcaaaggccaaggagggagttgtggc

SEQ ID NO: 577 - DNA, 100 bp, Artificial Sequence
  Description of Artificial Sequence: Synthetic polynucleotide
gccacaactccgaggaacccctttgaaagtcctttcacgaatacatccacggctaaacttctcctttacaccacactgtcgtcgaatggccactcccagt

SEQ ID NO: 578 - DNA, 100 bp, Artificial Sequence
  Description of Artificial Sequence: Synthetic polynucleotide
gccacaactccgaggaacccctttgaaagtcctttcacgaatacatccacggctaaacttctcctttacaccacactggaaaacataaaatacactttga

SEQ ID NO: 579 - DNA, 335 bp, Artificial Sequence
  Description of Artificial Sequence: Synthetic polynucleotide
  modified_base (144)..(148): a, c, t, g, unknown or other
tttctcagcagcagccacaactccgaggaacccctttgaaagtcctttcatgaatacatccacggctaaacttctcctttacaccacattagccagaaggcttgaaggcaaggcgtgagggagcgcccaggacgctctcggagnnnnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaa

SEQ ID NO: 580 - DNA, 187 bp, Artificial Sequence
  Description of Artificial Sequence: Synthetic polynucleotide
ctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaa

SEQ ID NO: 581 - DNA, 169 bp, Artificial Sequence
  Description of Artificial Sequence: Synthetic polynucleotide
  modified_base (169)..(169): a, c, t, g, unknown or other
tttctcagcagcagccacaactccgaggaacccctttgaaagtcctttcatgaatacatccacggctaaacttctcctttacaccacactggaaaacataaaatacactttgattagccagaaggcttgaaggcaaggcgtgagggagcgcccaggacgctctcggagn

SEQ ID NO: 582 - DNA, 190 bp, Artificial Sequence
  Description of Artificial Sequence: Synthetic polynucleotide
  modified_base (1)..(3): a, c, t, g, unknown or other
nnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaa

SEQ ID NO: 583 - DNA, 153 bp, Artificial Sequence
  Description of Artificial Sequence: Synthetic polynucleotide
tttctcagcagcagccacaactccgaggaacccctttgaaagtcctttcatgaatacatccacggctaaacttctcctttacaccacattagcaaaggcagaaggcttgaaggcaaggacaccgggagcgcccaggacgctctcggaggggcc

SEQ ID NO: 584 - DNA, 192 bp, Artificial Sequence
  Description of Artificial Sequence: Synthetic polynucleotide
  modified_base (1)..(5): a, c, t, g, unknown or other
nnnnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaa

SEQ ID NO: 585 - DNA, 178 bp, Artificial Sequence
  Description of Artificial Sequence: Synthetic polynucleotide
tttctcagcagcagccacaactccgaggaacccctttgaaagtcctttcatgaatacatccacggctaaacttctcctttacaccacactggaaaacataaaatacactttgattagcaaaggcagaaggcttgaaggcaaggacaccgggagcgcccaggacgctctcggaggggcc

SEQ ID NO: 586 - DNA, 371 bp, Artificial Sequence
  Description of Artificial Sequence: Synthetic polynucleotide
  modified_base (168)..(172): a, c, t, g, unknown or other
accctgtttggtttctcagcagcaggggagtggccctccttggcctttgaaagtcctttcatgaatacatccacggctaaacttctcctttacaccacattagcaaaggcagaaggcttgaaggcaaggacaccgggagcgcccaggacgctctcggaggggccgggnnnnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggt

SEQ ID NO: 587 - DNA, 204 bp, Artificial Sequence
  Description of Artificial Sequence: Synthetic polynucleotide
  modified_base (1)..(5): a, c, t, g, unknown or other
nnnnnctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggt

SEQ ID NO: 588 - DNA, 189 bp, Artificial Sequence
  Description of Artificial Sequence: Synthetic polynucleotide
accctgtttggtttctcagcagcaggggagtggccctccttggcctttgaaagtcctttcatgaatacatccacggctaaacttctcctttacaccacactggaaaacataaaatacactttgattagcaaaggcagaaggcttgaaggcaaggacaccgggagcgcccaggacgctctcggaggggcc

SEQ ID NO: 589 - DNA, 200 bp, Artificial Sequence
  Description of Artificial Sequence: Synthetic polynucleotide
cacctgtccgacatacactattcaagcgggacctcgcacgaaaccggaggaccctcacttaccacggccgttttgttggtgtaagttctacgagtgacccgggtcactcgtagaacttacaccaacaaaacggccgtggtaagtgagggtcctccggtttcgtgagaggtcccgcttgaagctgtgtgtcggacaggtga

SEQ ID NO: 590 - DNA, 258 bp, Artificial Sequence
  Description of Artificial Sequence: Synthetic polynucleotide
  modified_base (141)..(146): a, c, t, g, unknown or other
taatagggatagggatagggacaactccctccttggcctttgaaagtcctttcatgaatacatccacggctaatgtgggatactataacaacatttagcatattgttatactattccacgaattcctttacaccacatatnnnnnnactgggagtggccattcgacgacagtgtggtgtaaaggaattcattagccatggatgtattcatgaaaggactttcaaaggccaaggagggagttgtggctgctgctgagaa

SEQ ID NO: 591 - DNA, 119 bp, Artificial Sequence
  Description of Artificial Sequence: Synthetic polynucleotide
  modified_base (1)..(6): a, c, t, g, unknown or other
nnnnnnactgggagtggccattcgacgacagtgtggtgtaaaggaattcattagccatggatgtattcatgaaaggactttcaaaggccaaggagggagttgtggctgctgctgagaaa

SEQ ID NO: 592 - DNA, 158 bp, Artificial Sequence
  Description of Artificial Sequence: Synthetic polynucleotide
accgataatcaactccctccttggcctttgaaagtcctttcatgaatacaccctatgatattgttgtaaatcgtataacaatatgataaggtgcatccacggctaatgaattcctttacaccacactgtcgtcgaatggccactcccagttctcatat

SEQ ID NO: 593 - DNA, 289 bp, Artificial Sequence
  Description of Artificial Sequence: Synthetic polynucleotide
  modified_base (172)..(176): a, c, t, g, unknown or other
ccgataatagggatagggatagggacaactccctccttggcctttgaaagtcctttcatgaatacatccatagctaatgaattcctttacaccacactgtcgtcgaatggccactcccagttctcatataatttttggagcaggttttctgacttcggtcggaaaacccctnnnnnactgggagtggccattcgacgacagtgtggtgtaaaggaattcattagccatggatgtattcatgaaaggactttcaaaggccaaggagggagttgtggctgctgctgagaaa

SEQ ID NO: 594 - DNA, 271 bp, Artificial Sequence
  Description of Artificial Sequence: Synthetic polynucleotide
  modified_base (166)..(171): a, c, t, g, unknown or other
taatagggatagggatagggaccctgtttggttctctcagcagcagccacaactccctccttggcctttgaaagtcctttcatgaatacatccatagctaatgaattcctttacaccacatataatttttggagcaggttttctgacttcggtcggaaaacccctnnnnnntgtggtgtaaaggaattcattagccatggatgtattcatgaaaggactttcaaaggccaaggagggagttgtggctgctgctgagaaaaccaaacagggt

SEQ ID NO: 595 - RNA, 165 nt, Artificial Sequence
  Description of Artificial Sequence: Synthetic polynucleotide
uugugaaagaaggacgggucgucugguugugucauugcaaagauugcugacuacagcauugcucaguacugcuguaauaugcuaaauguuacagcaguacugagcaaugcuguagucagcaaucuuugcaaugagucugguugugccugugacuaacugugccaa

SEQ ID NO: 596 - DNA, 165 bp, Artificial Sequence
  Description of Artificial Sequence: Synthetic polynucleotide
ttgtgaaagaaggacgggtcgtctggttgtgtcattgcaaagattgctgactacagcattgctcagtactgctgtaatatgctaaatgttacagcagtactgagcaatgctgtagtcagcaatctttgcaatgagtctggttgtgcctgtgactaactgtgccaa

SEQ ID NO: 597 - RNA, 165 nt, Artificial Sequence
  Description of Artificial Sequence: Synthetic polynucleotide
uugugaaagaaggacgggucgugcguuguggucauugcaaagauugcugacuacagcauugcucaguacugcuguaauaugcuaaauguuacagcaguacugagcaaugccguagucagcaaucuuugcaaugagugcguuguggccugugacuaacugugccaa

SEQ ID NO: 598 - DNA, 165 bp, Artificial Sequence
  Description of Artificial Sequence: Synthetic polynucleotide
ttgtgaaagaaggacgggtcgtgcgttgtggtcattgcaaagattgctgactacagcattgctcagtactgctgtaatatgctaaatgttacagcagtactgagcaatgccgtagtcagcaatctttgcaatgagtgcgttgtggcctgtgactaactgtgccaa

SEQ ID NO: 599 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaguacuggguggugccguagucagcaaucuuugcaaugaccucuuguucgccugugacuaacugugccaa

SEQ ID NO: 600 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcagtactgggtggtgccgtagtcagcaatctttgcaatgacctcttgttcgcctgtgactaactgtgccaa

SEQ ID NO: 601 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuacagcaggacugagcagugccguagugucgaaucuuugcaaugaccuucucuccgccugugacuaacugugccaa

SEQ ID NO: 602 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttacagcaggactgagcagtgccgtagtgtcgaatctttgcaatgaccttctctccgcctgtgactaactgtgccaa

SEQ ID NO: 603 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuaaagcaggacugaguagugccguagucagcaaucuuugcaaugaccucuuguucgccugugacuaacugugccaa

SEQ ID NO: 604 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttaaagcaggactgagtagtgccgtagtcagcaatctttgcaatgacctcttgttcgcctgtgactaactgtgccaa

SEQ ID NO: 605 - RNA, 165 nt, Artificial Sequence
  Description of Artificial Sequence: Synthetic polynucleotide
uugugaaagaaggacgggucgugguuguucgucauugcaaagauugcugacuacagcauugcucaguacugcuguaauaugcuaaauguuacagcaguacugagcagugccguagugucguuucuuugcaaugagugguuguucgccugugacuaacugugccaa

SEQ ID NO: 606 - DNA, 165 bp, Artificial Sequence
  Description of Artificial Sequence: Synthetic polynucleotide
ttgtgaaagaaggacgggtcgtggttgttcgtcattgcaaagattgctgactacagcattgctcagtactgctgtaatatgctaaatgttacagcagtactgagcagtgccgtagtgtcgtttctttgcaatgagtggttgttcgcctgtgactaactgtgccaa

SEQ ID NO: 607 - RNA, 165 nt, Artificial Sequence
  Description of Artificial Sequence: Synthetic polynucleotide
uugugaaagaaggacgggucgugguuguucgucauugcaaagauugcugacuacagcauugcucaguacugcuguaauaugcuaaauguuacagcauuacugagcagugccguagugucguuucuuugcaaugagugguuguucgccugugacuaacugugccaa

SEQ ID NO: 608 - DNA, 165 bp, Artificial Sequence
  Description of Artificial Sequence: Synthetic polynucleotide
ttgtgaaagaaggacgggtcgtggttgttcgtcattgcaaagattgctgactacagcattgctcagtactgctgtaatatgctaaatgttacagcattactgagcagtgccgtagtgtcgtttctttgcaatgagtggttgttcgcctgtgactaactgtgccaa

SEQ ID NO: 609 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuacagcaggacugaguagugccguagugucgaaucuuugcaaugaccuucucuccgccugugacuaacugugccaa

SEQ ID NO: 610 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttacagcaggactgagtagtgccgtagtgtcgaatctttgcaatgaccttctctccgcctgtgactaactgtgccaa

SEQ ID NO: 611 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaggacugagcggugccguagucagcaaucuuugcaaugaccucuuguucgccugugacuaacugugccaa

SEQ ID NO: 612 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcaggactgagcggtgccgtagtcagcaatctttgcaatgacctcttgttcgcctgtgactaactgtgccaa

SEQ ID NO: 613 - RNA, 20 nt, Artificial Sequence
  Description of Artificial Sequence: Synthetic oligonucleotide
uugugaaagaaggacggguc

SEQ ID NO: 614 - RNA, 145 nt, Artificial Sequence
  Description of Artificial Sequence: Synthetic polynucleotide
ccucuuguucgucauugcaaagauugcugacuacagcauugcucaguacugcuguaauaugcuaaauguuacagcaguacugagcggugccguagucagcaaucuuugcaaugaccucuuguucgccugugacuaacugugccaa

SEQ ID NO: 615 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcagtactgagcggtgccgtagtcagcaatctttgcaatgacctcttgttcgcctgtgactaactgtgccaa

SEQ ID NO: 616 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaggacugggugaugccguagucagcaaucuuugcaaugaccucuuguucgccugugacuaacugugccaa

SEQ ID NO: 617 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcaggactgggtgatgccgtagtcagcaatctttgcaatgacctcttgttcgcctgtgactaactgtgccaa

SEQ ID NO: 618 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuaaagcaguacugagcggugccguagucagcaaucuuugcaaugaccucuuguucgccugugacuaacugugccaa

SEQ ID NO: 619 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttaaagcagtactgagcggtgccgtagtcagcaatctttgcaatgacctcttgttcgcctgtgactaactgtgccaa

SEQ ID NO: 620 - RNA, 145 nt, Artificial Sequence
  Description of Artificial Sequence: Synthetic polynucleotide
ccucuuguucgucauugcaaagauugcugacuacagcauugcucaguacugcuguaauaugcuaaauguuacagcaguccuggguggugccguagucagcaaucuuugcaaugaccucuuguucgccugugacuaacugugccaa

SEQ ID NO: 621 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcagtcctgggtggtgccgtagtcagcaatctttgcaatgacctcttgttcgcctgtgactaactgtgccaa

SEQ ID NO: 622 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuacagcaggacuggguagugccguagugucgaaucuuugcaaugaccuucucuccgccugugacuaacugugccaa

SEQ ID NO: 623 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttacagcaggactgggtagtgccgtagtgtcgaatctttgcaatgaccttctctccgcctgtgactaactgtgccaa

SEQ ID NO: 624 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaggacugagcagugccguagucagcaaucuuugcaaugaccucuuguucgccugugacuaacugugccaa

SEQ ID NO: 625 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcaggactgagcagtgccgtagtcagcaatctttgcaatgacctcttgttcgcctgtgactaactgtgccaa

SEQ ID NO: 626 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuaaagcaguacugagcagugccguagucagcaaucuuugcaaugaccucuuguucgccugugacuaacugugccaa

SEQ ID NO: 627 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttaaagcagtactgagcagtgccgtagtcagcaatctttgcaatgacctcttgttcgcctgtgactaactgtgccaa

SEQ ID NO: 628 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuaaagcaggccugagcagugccguagugucgaaucuuugcaaugaccuucucuccgccugugacuaacugugccaa

SEQ ID NO: 629 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttaaagcaggcctgagcagtgccgtagtgtcgaatctttgcaatgaccttctctccgcctgtgactaactgtgccaa

SEQ ID NO: 630 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuaaagcaggacugggcagugccguagugucgaaucuuugcaaugaccuucucuccgccugugacuaacugugccaa

SEQ ID NO: 631 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttaaagcaggactgggcagtgccgtagtgtcgaatctttgcaatgaccttctctccgcctgtgactaactgtgccaa

SEQ ID NO: 632 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaggacugggcgaugccguagucagcaaucuuugcaaugaccucuuguucgccugugacuaacugugccaa

SEQ ID NO: 633 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcaggactgggcgatgccgtagtcagcaatctttgcaatgacctcttgttcgcctgtgactaactgtgccaa

SEQ ID NO: 634 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaggacugggcagugccguagucagcaaucuuugcaaugaccucuuguucgccugugacuaacugugccaa

SEQ ID NO: 635 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcaggactgggcagtgccgtagtcagcaatctttgcaatgacctcttgttcgcctgtgactaactgtgccaa

SEQ ID NO: 636 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuaaagcaggacugggcagugccguagucagcaaucuuugcaaugaccucuuguucgccugugacuaacugugccaa

SEQ ID NO: 637 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttaaagcaggactgggcagtgccgtagtcagcaatctttgcaatgacctcttgttcgcctgtgactaactgtgccaa

SEQ ID NO: 638 - RNA, 165 nt, Artificial Sequence
  Description of Artificial Sequence: Synthetic polynucleotide
uugugaaagaaggacgggucgugguuguucgucauugcaaagauugcugacuacagcauugcucaguacugcuguaauaugcuaaauguuacagcauuacggagcagugccguagugucguuucuuugcaaugagugguuguucgccugugacuaacugugccaa

SEQ ID NO: 639 - DNA, 165 bp, Artificial Sequence
  Description of Artificial Sequence: Synthetic polynucleotide
ttgtgaaagaaggacgggtcgtggttgttcgtcattgcaaagattgctgactacagcattgctcagtactgctgtaatatgctaaatgttacagcattacggagcagtgccgtagtgtcgtttctttgcaatgagtggttgttcgcctgtgactaactgtgccaa

SEQ ID NO: 640 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuacagcaggccugagcagugccguagugucgaaucuuugcaaugaccuucucuccgccugugacuaacugugccaa

SEQ ID NO: 641 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttacagcaggcctgagcagtgccgtagtgtcgaatctttgcaatgaccttctctccgcctgtgactaactgtgccaa

SEQ ID NO: 642 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuaaagcaggacuggguagugccguagugucgaaucuuugcaaugaccuucucuccgccugugacuaacugugccaa

SEQ ID NO: 643 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttaaagcaggactgggtagtgccgtagtgtcgaatctttgcaatgaccttctctccgcctgtgactaactgtgccaa

SEQ ID NO: 644 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuacagcaguacugagcagugccguagugucgaaucuuugcaaugaccuucucuccgccugugacuaacugugccaa

SEQ ID NO: 645 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttacagcagtactgagcagtgccgtagtgtcgaatctttgcaatgaccttctctccgcctgtgactaactgtgccaa

SEQ ID NO: 646 - RNA, 165 nt, Artificial Sequence
  Description of Artificial Sequence: Synthetic polynucleotide
uugugaaagaaggacgggucgugcguuguggucauugcaaagauugcugacuacagcauugcucaguacugcuguaauaugcuaaauguuacagcaauaccgagcagugccguagucagcaaucuuugcaaugagugcguuguggccugugacuaacugugccaa

SEQ ID NO: 647 - DNA, 165 bp, Artificial Sequence
  Description of Artificial Sequence: Synthetic polynucleotide
ttgtgaaagaaggacgggtcgtgcgttgtggtcattgcaaagattgctgactacagcattgctcagtactgctgtaatatgctaaatgttacagcaataccgagcagtgccgtagtcagcaatctttgcaatgagtgcgttgtggcctgtgactaactgtgccaa

SEQ ID NO: 648 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaguacugggcggugccguagucagcaaucuuugcaaugaccucuuguucgccugugacuaacugugccaa

SEQ ID NO: 649 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcagtactgggcggtgccgtagtcagcaatctttgcaatgacctcttgttcgcctgtgactaactgtgccaa

SEQ ID NO: 650 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaggacuggguggugccguagucagcaaucuuugcaaugaccucuuguucgccugugacuaacugugccaa

SEQ ID NO: 651 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcaggactgggtggtgccgtagtcagcaatctttgcaatgacctcttgttcgcctgtgactaactgtgccaa

SEQ ID NO: 652 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuaaagcaggacugagcagugccguagucagcaaucuuugcaaugaccucuuguucgccugugacuaacugugccaa

SEQ ID NO: 653 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttaaagcaggactgagcagtgccgtagtcagcaatctttgcaatgacctcttgttcgcctgtgactaactgtgccaa

SEQ ID NO: 654 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuaaagcaggacugaguagugccguagugucgaaucuuugcaaugaccuucucuccgccugugacuaacugugccaa

SEQ ID NO: 655 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttaaagcaggactgagtagtgccgtagtgtcgaatctttgcaatgaccttctctccgcctgtgactaactgtgccaa

SEQ ID NO: 656 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaguacugaguggugccguagucagcaaucuuugcaaugaccucuuguucgccugugacuaacugugccaa

SEQ ID NO: 657 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcagtactgagtggtgccgtagtcagcaatctttgcaatgacctcttgttcgcctgtgactaactgtgccaa

SEQ ID NO: 658 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaggacugggcggugccguagucagcaaucuuugcaaugaccucuuguucgccugugacuaacugugccaa

SEQ ID NO: 659 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcaggactgggcggtgccgtagtcagcaatctttgcaatgacctcttgttcgcctgtgactaactgtgccaa

SEQ ID NO: 660 - RNA, 165 nt, Artificial Sequence
  Description of Artificial Sequence: Synthetic polynucleotide
uugugaaagaaggacgggucccucuuguucgucauugcaaagauugcugacuacagcauugcucaguacugcuguaauaugcuaaauguuacagcaguccugggcggugccguagucagcaaucuuugcaaugaccucuuguucgccugugacuaacugugccaa

SEQ ID NO: 661 - DNA, 165 bp, Artificial Sequence
  Description of Artificial Sequence: Synthetic polynucleotide
ttgtgaaagaaggacgggtccctcttgttcgtcattgcaaagattgctgactacagcattgctcagtactgctgtaatatgctaaatgttacagcagtcctgggcggtgccgtagtcagcaatctttgcaatgacctcttgttcgcctgtgactaactgtgccaa

SEQ ID NO: 662 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuaaagcaguacugagcagugccguagugucgaaucuuugcaaugaccuucucuccgccugugacuaacugugccaa

SEQ ID NO: 663 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttaaagcagtactgagcagtgccgtagtgtcgaatctttgcaatgaccttctctccgcctgtgactaactgtgccaa

SEQ ID NO: 664 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuaaagcaggacugagcagugccguagugucgaaucuuugcaaugaccuucucuccgccugugacuaacugugccaa

SEQ ID NO: 665 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttaaagcaggactgagcagtgccgtagtgtcgaatctttgcaatgaccttctctccgcctgtgactaactgtgccaa

SEQ ID NO: 666 - RNA, 165 nt, Artificial Sequence
  Description of Artificial Sequence: Synthetic polynucleotide
uugugaaagaaggacgggucccuucucuccgucauugcaaagauugcugacuacagcauugcucaguacugcuguaauaugcuaaauguuacagcaggacugggcagugccguagugucgaaucuuugcaaugaccuucucuccgccugugacuaacugugccaa

SEQ ID NO: 667 - DNA, 165 bp, Artificial Sequence
  Description of Artificial Sequence: Synthetic polynucleotide
ttgtgaaagaaggacgggtcccttctctccgtcattgcaaagattgctgactacagcattgctcagtactgctgtaatatgctaaatgttacagcaggactgggcagtgccgtagtgtcgaatctttgcaatgaccttctctccgcctgtgactaactgtgccaa

SEQ ID NO: 668 - DNA, 121 bp, Artificial Sequence
  Description of Artificial Sequence: Synthetic polynucleotide
gactgacgttgcaaagattgctgactacagcattgctcagtactgctgtagaatataggctaaatcgattctaaagcaggacggggtggtgccgtagtgtcgtatctttgcacttacagtc

SEQ ID NO: 669 - DNA, 121 bp, Artificial Sequence
  Description of Artificial Sequence: Synthetic polynucleotide
gactgacgttgcaaagattgctgactacagcattgctcagtactgctgtagaatataggctaaatcgattctaaagcaggactgggtggtgccgtagtgtcgtatctttgcacttacagtc

SEQ ID NO: 670 - DNA, 121 bp, Artificial Sequence
  Description of Artificial Sequence: Synthetic polynucleotide
gactgacgttgcaaagattgctgactacagcattgctcagtactgctgtagaatataggctaaatcgattctaaagcaggaccgggtggtgccgtagtgtcgtatctttgcacttacagtc

SEQ ID NO: 671 - DNA, 121 bp, Artificial Sequence
  Description of Artificial Sequence: Synthetic polynucleotide
gactgacgttgcaaagattgctgactacagcattgctcagtactgctgtagaatataggctaaatcgattctagagcaggactgggtggtgccgtagtgtcgtatctttgcacttacagtc

SEQ ID NO: 672 - DNA, 121 bp, Artificial Sequence
  Description of Artificial Sequence: Synthetic polynucleotide
gactgacgttgcaaagattgctgactacagcattgctcagtactgctgtagaatataggctaaatcgattctacagcaggactgggtggtgccgtagtgtcgtatctttgcacttacagtc

SEQ ID NO: 673 - DNA, 121 bp, Artificial Sequence
  Description of Artificial Sequence: Synthetic polynucleotide
gactgacgttgcaaagattgctgactacagcattgctcagtactgctgtagaatataggctaaatcgattctaaagcatgactgggtggtgccgtagtcagcaatctttgcacttacagtc

SEQ ID NO: 674 - DNA, 121 bp, Artificial Sequence
  Description of Artificial Sequence: Synthetic polynucleotide
gactgacgttgcaaagattgctgactacagcattgctcagtactgctgtagaatataggctaaatcgattctaaagcaggactgggtggtgccgtagtcagcaatctttgcacttacagtc

SEQ ID NO: 675 - DNA, 121 bp, Artificial Sequence
  Description of Artificial Sequence: Synthetic polynucleotide
gactgacgttgcaaagattgctgactacagcattgctcagtactgctgtagaatataggctaaatcgattctaaagcagtactgggtggtgccgtagtcagcaatctttgcacttacagtc

SEQ ID NO: 676 - DNA, 121 bp, Artificial Sequence
  Description of Artificial Sequence: Synthetic polynucleotide
gactgacgttgcaaagattgctgactacagcattgctcagtactgctgtagaatataggctaaatcgattctagagcatgactgggtggtgccgtagtcagcaatctttgcacttacagtc

SEQ ID NO: 677 - DNA, 121 bp, Artificial Sequence
  Description of Artificial Sequence: Synthetic polynucleotide
gactgacgttgcaaagattgctgactacagcattgctcagtactgctgtagaatataggctaaatcgattctagagcaggacggggtggtgccgtagtcagcaatctttgcacttacagtc

SEQ ID NO: 678 - DNA, 231 bp, Artificial Sequence
  Description of Artificial Sequence: Synthetic polynucleotide
acgacgaaaagtgtgacatagggttacgacgcatcatacgtttctaacgggtcgagccgtcacgagtcaggatgacatcttaggggattttttgtagtctcccgtgtggtcccaaaaagggaccacacgggagactacaaaaataggggtaagatgtcgtcatgactcgttacgacatcagtcgttagaaacgttactaccgtcgtaaccctatgtcacacttttcgtcgt

SEQ ID NO: 679 - DNA, 231 bp, Artificial Sequence
  Description of Artificial Sequence: Synthetic polynucleotide
acgacgaaaagtgtgacatagggttacgacgcatcatacgtttctaaccactggagccgtcaggagacatgacgacatcttaggggattttttgtagtctcccgtgtggtcccaaaaagggaccacacgggagactacaaaaataggggtaagatgtcgtcatgactcgttacgacatcagtcgttagaaacgttactaccgtcgtaaccctatgtcacacttttcgtcgt

SEQ ID NO: 680 - DNA, 231 bp, Artificial Sequence
  Description of Artificial Sequence: Synthetic polynucleotide
acgacgaaaagtgtgacatagggttacgacgcatcatacgtttctaacaacttaagccctatcgagtcatgacaacatcttaggggattttttgtagtctcccgtgtggtcccaaaaagggaccacacgggagactacaaaaataggggtaagatgtcgtcatgactcgttacgacatcagtcgttagaaacgttactaccgtcgtaaccctatgtcacacttttcgtcgt

SEQ ID NO: 681 - DNA, 231 bp, Artificial Sequence
  Description of Artificial Sequence: Synthetic polynucleotide
acgacgaaaagtgtgacatagggttacgacgcatcatacgtttctaacgactggagccctaaggaggcttgacgacatcttcggggattttttgtagtctcccgtgtggtcccaaaaagggaccacacgggagactacaaaaataggggtaagatgtcgtcatgactcgttacgacatcagtcgttagaaacgttactaccgtcgtaaccctatgtcacacttttcgtcgt

SEQ ID NO: 682 - DNA, 231 bp, Artificial Sequence
  Description of Artificial Sequence: Synthetic polynucleotide
acgacgaaaagtgtgacatagggttacgacgcatcatacgtttctaacgattgatgccctaaccagtcatggcggcatcttaggggattttttgtagtctcccgtgtggtcccaaaaagggaccacacgggagactacaaaaataggggtaagatgtcgtcatgactcgttacgacatcagtcgttagaaacgttactaccgtcgtaaccctatgtcacacttttcgtcgt

SEQ ID NO: 683 - DNA, 231 bp, Artificial Sequence
  Description of Artificial Sequence: Synthetic polynucleotide
acgacgaaaagtgtgacatagggttacgacgcatcatacgtttctaacgactgatgccctagcccatcatgacgccatcttaggggattttttgtagtctcccgtgtggtcccaaaaagggaccacacgggagactacaaaaataggggtaagatgtcgtcatgactcgttacgacatcagtcgttagaaacgttactaccgtcgtaaccctatgtcacacttttcgtcgt

SEQ ID NO: 684 - DNA, 231 bp, Artificial Sequence
  Description of Artificial Sequence: Synthetic polynucleotide
acgacgaaaagtgtgacatagggttacgacgcatcatccgtttctaacaactgatgccctaacgatacttgacggcatcttaggggattttttgtagtctcccgtgtggtcccaaaaagggaccacacgggagactacaaaaataggggtaagatgtcgtcatgactcgttacgacatcagtcgttagaaacgttactaccgtcgtaaccctatgtcacacttttcgtcgt

SEQ ID NO: 685 - RNA, 103 nt, Artificial Sequence
  Description of Artificial Sequence: Synthetic polynucleotide
agcaguggacaggcugugugucgaaguucgcccuggauaggugggauaugcuaaaugucccaccucuccagggcgaauuuggacauacagccuguccacugcu

SEQ ID NO: 686 - RNA, 103 nt, Artificial Sequence
  Description of Artificial Sequence: Synthetic polynucleotide
agcaguggacaggcugugugucgaaguucgcccuggagaggugggauaugcuaaaugucccaccucuucagggcgaacuuggacacacagccuguccacugcu

SEQ ID NO: 687 - RNA, 103 nt, Artificial Sequence
  Description of Artificial Sequence: Synthetic polynucleotide
agcaguggacaggcugugugucgaaguucgcccuggagaggugggauaugcuaaaugucccaccgcuccagggcgaauuugggcauacagccuguccacugcu

SEQ ID NO: 688 - RNA, 103 nt, Artificial Sequence
  Description of Artificial Sequence: Synthetic polynucleotide
agcaguggacaggcugugugucgaaguucgcccugaagaggugggauaugcuaaauauccgaccucuucagggcgaucuuggacacacagccuguccacugcu

SEQ ID NO: 689 - RNA, 103 nt, Artificial Sequence
  Description of Artificial Sequence: Synthetic polynucleotide
agcaguggacaggcugugugucgaaguucgcccuggagaggugggauaugcuaaaugucccaccucuccagggcgaucuuggacacacaaccuguccacugcu

SEQ ID NO: 690 - RNA, 103 nt, Artificial Sequence
  Description of Artificial Sequence: Synthetic polynucleotide
agcaguggacaggcugugugucgaaguucgcccuggagaggugggauaugcuaaauaucccaccucuccagggcgaauuuggucauacagccuguccacugcu

SEQ ID NO: 691 - RNA, 103 nt, Artificial Sequence
  Description of Artificial Sequence: Synthetic polynucleotide
agcaguggacaagcugugugucgaaguucgcccuggaaaggugggauaugcuaaauaucccaccucuccagggcgaucuuggacacacagccuguccacugcu

SEQ ID NO: 692 - RNA, 103 nt, Artificial Sequence
  Description of Artificial Sequence: Synthetic polynucleotide
agcaguggacaggcugugugucgaaguucgcccugaagaggugggauaugcuaaauguccgaccucuucagggcgaucuuggacacacaaccuguccacugcu

SEQ ID NO: 693 - RNA, 103 nt, Artificial Sequence
  Description of Artificial Sequence: Synthetic polynucleotide
agcaguggacaggcuguaugucgaaguucgcccuggagaggugggauaugcuaaaugucccaccucuccagggcgaacuuggacauacagccuguccacugcu

SEQ ID NO: 694 - RNA, 103 nt, Artificial Sequence
  Description of Artificial Sequence: Synthetic polynucleotide
agcaguggacaggcugugugucgaaguucgcccugaagaggugggauaugcuaaauauccgaccucuucagggcgaacucggacacacagccuguccacugcu

SEQ ID NO: 695 - RNA, 101 nt, Artificial Sequence
  Description of Artificial Sequence: Synthetic polynucleotide
agcaguggacaggcugugugucgaaguucgcccuggagaggugggauaugcuaaauaucccaccucuccgggacgaacucggacaacagguguccacugcu

SEQ ID NO: 696 - RNA, 103 nt, Artificial Sequence
  Description of Artificial Sequence: Synthetic polynucleotide
agcaguggacaggcugugugucgaaguucgcccuggagaggugggauaugcuaaauaucccaccucuccagggcgaauuuggacacacagccuguccacugcu

SEQ ID NO: 697 - RNA, 104 nt, Artificial Sequence
  Description of Artificial Sequence: Synthetic polynucleotide
agcaguggacaggcugugugucgaaguucgcccuggagaggugggauaugcuaaauaucccaccgcuccagggcgaacuuaucacauacagccuguccacugcu

SEQ ID NO: 698 - RNA, 103 nt, Artificial Sequence
  Description of Artificial Sequence: Synthetic polynucleotide
agcaguggacaggcuguaugucgaaguucgcccuggagaggugggauaugcuaaaugucccaccucuccagggcgaacuuggucauacagccuguccacugcu

SEQ ID NO: 699 - RNA, 103 nt, Artificial Sequence
  Description of Artificial Sequence: Synthetic polynucleotide
agcaguggacaggcugugugucgaaauucgcccuggagaggugggauaugcuaaauaucccaccgcuccagggcgaauuuggacacacagccuguccacugcu

SEQ ID NO: 700 - RNA, 103 nt, Artificial Sequence
  Description of Artificial Sequence: Synthetic polynucleotide
agcaguggacaggcuguaugucgaaguucgcccuggagaggugggauaugcuaaauaucccaccgcuccagggcgaacuuggucauacagccuguccacugcu

SEQ ID NO: 701 - RNA, 103 nt, Artificial Sequence
  Description of Artificial Sequence: Synthetic polynucleotide
agcaguggacaggcugugugucgaaauucgcccuggagaggugggauaugcuaaaugucccaccgcuccagggcgaauuuggucacacagccuguccacugcu

SEQ ID NO: 702 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccacctctccagggcgaacttcgacacacagcctgtccactgctgccgcttcttgcctgtgactaactgtgccaa

SEQ ID NO: 703 - DNA, 165 bp, Artificial Sequence
  Description of Artificial Sequence: Synthetic polynucleotide
ttgtgaaagaaggacgggtcggttgttgtggagcagtggacaggctgtgtgtcgaagttcgccctggagaggtgggatatgctaaatgtcccacctctccagggcgaactccgacacacagcctgtccactgctggttgttgtggcctgtgactaactgtgccaa

SEQ ID NO: 704 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccacctctccagggcgaatttggacatacagcctgtccactgctgccgcttcttgcctgtgactaactgtgccaa

SEQ ID NO: 705 - DNA, 163 bp, Artificial Sequence
  Description of Artificial Sequence: Synthetic polynucleotide
ttgtgaaagaaggacgggtcggtctctctggagcagtggacaggctgtgtgtcgaagttcgccctggagaggtgggatatgctaaatgtcccacctctccgggacgaactcggacaacagttgtccactgctggtctctctggcctgtgactaactgtgccaa

SEQ ID NO: 706 - DNA, 163 bp, Artificial Sequence
  Description of Artificial Sequence: Synthetic polynucleotide
ttgtgaaagaaggacgggtcggtctctctggagcagtggacaggctgtgtgtcgaagttcgccctggagaggtgggatatgctaaatgtcccacctctccaggacgaactcggacaacagatgtccactgctggtctctctggcctgtgactaactgtgccaa

SEQ ID NO: 707 - DNA, 163 bp, Artificial Sequence
  Description of Artificial Sequence: Synthetic polynucleotide
ttgtgaaagaaggacgggtcggtctctctggagcagtggacaggctgtgtgtcgaagttcgccctggagaggtgggatatgctaaatgtcccacctctccggggcgaactcggacaacagttgtccactgctggtctctctggcctgtgactaactgtgccaa

SEQ ID NO: 708 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccacctctccagggcgaatttggtcacacagcctgtccactgctgccgcttcttgcctgtgactaactgtgccaa

SEQ ID NO: 709 - DNA, 163 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccacctctccagggcgaacttggacacacagcctgtccactgctgccgcttcttgcctgtgactaactgtgcc

SEQ ID NO: 710 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccacctctccagggcgaacttggacacacagcctgtccactgctgccgcttcttgcctgtgactaactgtgccaa

SEQ ID NO: 711 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccaccgctccagggcgaacttggtcacacagcctgtccactgctgccgcttcttgcctgtgactaactgtgccaa

SEQ ID NO: 712 - DNA, 163 bp, Artificial Sequence
  Description of Artificial Sequence: Synthetic polynucleotide
ttgtgaaagaaggacgggtcggtctctctggagcagtggacaggctgtgtgtcgaagttcgccctggagaggtgggatatgctaaatgtcccacctctccgggacgaactcggacaacagatgtccactgctggtctctctggcctgtgactaactgtgccaa

SEQ ID NO: 713 - DNA, 165 bp, Artificial Sequence
  Description of Artificial Sequence: Synthetic polynucleotide
ttgtgaaagaaggacgggtcggcgtgtcttgagcagtggacaggctgtgtgtcgaagttcgccctggagaggtgggatatgctaaatgtcccacctctccaggacgaactcggacacacaggctgtccactgctggcgtgtcttgcctgtgactaactgtgccaa

SEQ ID NO: 714 - DNA, 165 bp, Artificial Sequence
  Description of Artificial Sequence: Synthetic polynucleotide
ttgtgaaagaaggacgggtcggttgttgtggagcagtggacaggctgtgtgtcgaagttcgccctggagaggtgggatatgctaaatgtcccacctcttcagggcgatcttggacacacagcctgtccactgctggttgttgtggcctgtgactaactgtgccaa

SEQ ID NO: 715 - DNA, 163 bp, Artificial Sequence
  Description of Artificial Sequence: Synthetic polynucleotide
ttgtgaaagaaggacgggtcggtctctctggagcagtggacaggctgtgtgtcgaagttcgccctggagaggtgggatatgctaaatgtcccacctctccaggacgaactcggacaacagctgtccactgctggtctctctggcctgtgactaactgtgccaa

SEQ ID NO: 716 - DNA, 165 bp, Artificial Sequence
  Description of Artificial Sequence: Synthetic polynucleotide
ttgtgaaagaaggacgggtcggttgttgtggagcagtggacaggctgtgtgtcgaagttcgccctggagaggtgggatatgctaaatgtcccacctcttcagggcgaacttggacacacagcctgtccactgctggttgttgtggcctgtgactaactgtgccaa

SEQ ID NO: 717 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccaccgctccagggcgaacttggacatacagcctgtccactgctgccgcttcttgcctgtgactaactgtgccaa

SEQ ID NO: 718 - DNA, 163 bp, Artificial Sequence
  Description of Artificial Sequence: Synthetic polynucleotide
ttgtgaaagaaggacgggtcggtctctctggagcagtggacaggctgtgtgtcgaagttcgccctggagaggtgggatatgctaaatgtcccacctctccgggacgaactcggacaacagctgtccactgctggtctctctggcctgtgactaactgtgccaa

SEQ ID NO: 719 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccaccgctccagggcgaatttggtcatacagcctgtccactgctgccgcttcttgcctgtgactaactgtgccaa

SEQ ID NO: 720 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccaccgctccagggcgaatttggacatacagcctgtccactgctgccgcttcttgcctgtgactaactgtgccaa

SEQ ID NO: 721 - DNA, 165 bp, Artificial Sequence
  Description of Artificial Sequence: Synthetic polynucleotide
ttgtgaaagaaggacgggtcggttgttgtggagcagtggacaggctgtgtgtcgaagttcgccctggagaggtgggatatgctaaatgtccgacctcttcagggcgatcttggacacacagcctgtccactgctggttgttgtggcctgtgactaactgtgccaa

SEQ ID NO: 722 - DNA, 165 bp, Artificial Sequence
  Description of Artificial Sequence: Synthetic polynucleotide
ttgtgaaagaaggacgggtcggttgttgtggagcagtggacaggctgtgtgtcgaagttcgccctggagaggtgggatatgctaaatgtcccacctcttcagggcgatcttggacacacaacctgtccactgctggttgttgtggcctgtgactaactgtgccaa

SEQ ID NO: 723 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccacctctccagggcgaatttggtcatacagcctgtccactgctgccgcttcttgcctgtgactaactgtgccaa

SEQ ID NO: 724 - DNA, 163 bp, Artificial Sequence
  Description of Artificial Sequence: Synthetic polynucleotide
ttgtgaaagaaggacgggtcggtctctctggagcagtggacaggctgtgtgtcgaagttcgccctggagaggtgggatatgctaaatgtcccacctctccaggacgaactcggacaacagttgtccactgctggtctctctggcctgtgactaactgtgccaa

SEQ ID NO: 725 - DNA, 165 bp, Artificial Sequence
  Description of Artificial Sequence: Synthetic polynucleotide
ttgtgaaagaaggacgggtcggttgttgtggagcagtggacaggctgtgtgtcgaagttcgccctggagaggtgggatatgctaaatgtccgacctctccagggcgatcttggacacacagcctgtccactgctggttgttgtggcctgtgactaactgtgccaa

SEQ ID NO: 726 - DNA, 165 bp, Artificial Sequence
  Description of Artificial Sequence: Synthetic polynucleotide
ttgtgaaagaaggacgggtcggttgttgtggagcagtggacaggctgtgtgtcgaagttcgccctggagaggtgggatatgctaaatgtccgacctcttcagggcgatcttggacacacaacctgtccactgctggttgttgtggcctgtgactaactgtgccaa

SEQ ID NO: 727 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccacctctccagggcgaacttggacatacagcctgtccactgctgccgcttcttgcctgtgactaactgtgccaa

SEQ ID NO: 728 - DNA, 165 bp, Artificial Sequence
  Description of Artificial Sequence: Synthetic polynucleotide
ttgtgaaagaaggacgggtcggttgttgtggagcagtggacaggctgtgtgtcgaagttcgccctggagaggtgggatatgctaaatgtccgacctcttcagggcgaactcggacacacagcctgtccactgctggttgttgtggcctgtgactaactgtgccaa

SEQ ID NO: 729 - DNA, 165 bp, Artificial Sequence
  Description of Artificial Sequence: Synthetic polynucleotide
ttgtgaaagaaggacgggtcggcgtgtcttgagcagtggacaggctgtgtgtcgaagttcgccctggagaggtgggatatgctaaatgtcccacctctccgggacgaactcggacacacaggctgtccactgctggcgtgtcttgcctgtgactaactgtgccaa

SEQ ID NO: 730 - DNA, 163 bp, Artificial Sequence
  Description of Artificial Sequence: Synthetic polynucleotide
ttgtgaaagaaggacgggtcggtctctctggagcagtggacaggctgtgtgtcgaagttcgccctggagaggtgggatatgctaaatgtcccacctctccgggacgaactcggacaacaggtgtccactgctggtctctctggcctgtgactaactgtgccaa

SEQ ID NO: 731 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccacctctccagggcgaatttggacacacagcctgtccactgctgccgcttcttgcctgtgactaactgtgccaa

SEQ ID NO: 732 - DNA, 166 bp, Artificial Sequence
  Description of Artificial Sequence: Synthetic polynucleotide
ttgtgaaagaaggacgggtcgcgtctgtctgagcagtggacaggctgtgtgtcgaagttcgccctggagaggtgggatatgctaaatgtcccaccgctccagggcgaacttatcacatacagcctgtccactgctgcgtctgtctgcctgtgactaactgtgccaa

SEQ ID NO: 733 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccacctctccagggcgaacttggtcatacagcctgtccactgctgccgcttcttgcctgtgactaactgtgccaa

SEQ ID NO: 734 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccaccgctccagggcgaatttggacacacagcctgtccactgctgccgcttcttgcctgtgactaactgtgccaa

SEQ ID NO: 735 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccaccgctccagggcgaacttggtcatacagcctgtccactgctgccgcttcttgcctgtgactaactgtgccaa

SEQ ID NO: 736 - DNA, 165 bp, Artificial Sequence
  Description of Artificial Sequence: Synthetic polynucleotide
ttgtgaaagaaggacgggtcgccgcttcttgagcagtggacaggctgtgtgtcgaagttcgccctggagaggtgggatatgctaaatgtcccaccgctccagggcgaatttggtcacacagcctgtccactgctgccgcttcttgcctgtgactaactgtgccaa

SEQ ID NO: 737 - DNA, 75 bp, Artificial Sequence
  Description of Artificial Sequence: Synthetic oligonucleotide
accatcgacaagaaagggactgaagctgctgatatgctaaatgtcagcagcttcagtccctttctcgtcgatggt

SEQ ID NO: 738 - DNA, 84 bp, Artificial Sequence
  Description of Artificial Sequence: Synthetic oligonucleotide
gaccatcgacaagaaagggactgaagctgctgatatgctaaatgtcagcagcttcagtcccttactcgtcgatggtcgggtggg

SEQ ID NO: 739 - RNA, 50 nt, Artificial Sequence
  Description of Artificial Sequence: Synthetic oligonucleotide
cauaaggcugugcugaccaucgacaagaaagggacugaagcugcuggggc

SEQ ID NO: 740 - RNA, 50 nt, Artificial Sequence
  Description of Artificial Sequence: Synthetic oligonucleotide
gccccagcagcuucagucccuuacucgucgauggucagcacagccuuaug

SEQ ID NO: 741 - DNA, 198 bp, Artificial Sequence
  Description of Artificial Sequence: Synthetic polynucleotide
  modified_base (96)..(103): a, c, t, g, unknown or other
tctgcttctctcccctccaggccgtgcataaggctgtgctgaccatcgacaagaaagggactgaagctgctggggccatgtttttagaggccatannnnnnnntatggcctctaaaaacatggccccagcagcttcagtcccttactcgtcgatggtcagcacagccaaatgcacggcctggaggggagagaagcaga

SEQ ID NO: 742 - DNA, 201 bp, Artificial Sequence
  Description of Artificial Sequence: Synthetic polynucleotide
  modified_base (96)..(103): a, c, t, g, unknown or other
tctgcttctctcccctccaggccgtgcataaggctgtgctgaccatcgacaagaaagggactgaagctgctggggccatgtttttagaggccatannnnnnnntatggcctctaaaaacatggccccagcagcttcagtcccttactcgtcgatggtcagcacagccccagtatgcacggcctggaggggagagaagcaga

SEQ ID NO: 743 - DNA, 202 bp, Artificial Sequence
  Description of Artificial Sequence: Synthetic polynucleotide
  modified_base (96)..(103): a, c, t, g, unknown or other
tctgcttctctcccctccaggccgtgcataaggctgtgctgaccatcgacaagaaagggactgaagctgctggggccatgtttttagaggccatannnnnnnntatggcctctaaaaacatggccccagcagcttcagtcccttactcgtcgatggtcagcacagccagtcgtatgcacggcctggaggggagagaagcaga

SEQ ID NO: 744 - DNA, 231 bp, Artificial Sequence
  Description of Artificial Sequence: Synthetic polynucleotide
  modified_base (108)..(117): a, c, t, g, unknown or other
ctctgcttctctcccctccaggccgtgcataaggctgtgctgaccatcgacaagaaagggactgaagctgctggggccatgtttttagaggccatacccatgtctatnnnnnnnnnnatagacatgggtatggcctctccacaaaacatggccccagcagcttcagtcccttactcgtcgatggtcagcacagccagtcctatgcacggcctggaggggagagaagcagag

SEQ ID NO: 745 - DNA, 105 bp, Artificial Sequence
  Description of Artificial Sequence: Synthetic polynucleotide
  modified_base (1)..(1): a, c, t, g, unknown or other
nactgggagtggccattcgacgacagtgtggtgtaaaggaattcattagccatggatgtattcatgaaaggactttcaaaggccaaggagggagttgtggctgct

SEQ ID NO: 746 - DNA, 105 bp, Artificial Sequence
  Description of Artificial Sequence: Synthetic polynucleotide
  modified_base (105)..(105): a, c, t, g, unknown or other
agcagccacaactccgggaacccctttgaaagtcctttcccgaatacatccacggctaaacttctcctttacaccacactgtcgtcgattaggccactcccagtn

SEQ ID NO: 747 - RNA, 103 nt, Artificial Sequence
  Description of Artificial Sequence: Synthetic polynucleotide
ucauugcaaagauugcugacuacagcauugcucaguacugcuguaauaugcuaaauguuacagcaauaccgagcagugccguagucagcaaucuuugcaauga

SEQ ID NO: 748 - RNA, 103 nt, Artificial Sequence
  Description of Artificial Sequence: Synthetic polynucleotide
ucauugcaaagauugcugacuacagcauugcucaguacugcuguaauaugcuaaauguuacagcaguacugagcagugccguagugucguuucuuugcaauga

SEQ ID NO: 749 - RNA, 103 nt, Artificial Sequence
  Description of Artificial Sequence: Synthetic polynucleotide
ucauugcaaagauugcugacuacagcauugcucaguacugcuguaauaugcuaaauguuacagcauuacggagcagugccguagugucguuucuuugcaauga

SEQ ID NO: 750 - RNA, 103 nt, Artificial Sequence
  Description of Artificial Sequence: Synthetic polynucleotide
ucauugcaaagauugcugacuacagcauugcucaguacugcuguaauaugcuaaauguuacagcauuacugagcagugccguagugucguuucuuugcaauga

SEQ ID NO: 751 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaggacugagcagugccguagucagcaaucuuugcaauga

SEQ ID NO: 752 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaggacugaguagugccguagucagcaaucuuugcaauga

SEQ ID NO: 753 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaguacugagcagugccguagucagcaaucuuugcaauga

SEQ ID NO: 754 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaguacugagcggugccguagucagcaaucuuugcaauga

SEQ ID NO: 755 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacugagcagugccguagucagcaaucuuugcaauga

SEQ ID NO: 756 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacugagcggugccguagucagcaaucuuugcaauga

SEQ ID NO: 757 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacugggcagugccguagucagcaaucuuugcaauga

SEQ ID NO: 758 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacugggcgaugccguagucagcaaucuuugcaauga

SEQ ID NO: 759 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacugggcggugccguagucagcaaucuuugcaauga

SEQ ID NO: 760 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacugggugaugccguagucagcaaucuuugcaauga

SEQ ID NO: 761 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacuggguggugccguagucagcaaucuuugcaauga

SEQ ID NO: 762 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaggacugggcagugccguagucagcaaucuuugcaauga

SEQ ID NO: 763 - RNA, 103 nt, Artificial Sequence
  Description of Artificial Sequence: Synthetic polynucleotide
ucauugcaaagauugcugacuacagcauugcucaguacugcuguaauaugcuaaauguuacagcaguacugagcggugccguagucagcaaucuuugcaauga

SEQ ID NO: 764 - RNA, 103 nt, Artificial Sequence
  Description of Artificial Sequence: Synthetic polynucleotide
ucauugcaaagauugcugacuacagcauugcucaguacugcuguaauaugcuaaauguuacagcaguacugaguggugccguagucagcaaucuuugcaauga

SEQ ID NO: 765 - RNA, 103 nt, Artificial Sequence
  Description of Artificial Sequence: Synthetic polynucleotide
ucauugcaaagauugcugacuacagcauugcucaguacugcuguaauaugcuaaauguuacagcaguacugggcggugccguagucagcaaucuuugcaauga

SEQ ID NO: 766 - RNA, 103 nt, Artificial Sequence
  Description of Artificial Sequence: Synthetic polynucleotide
ucauugcaaagauugcugacuacagcauugcucaguacugcuguaauaugcuaaauguuacagcaguacuggguggugccguagucagcaaucuuugcaauga

SEQ ID NO: 767 - RNA, 103 nt, Artificial Sequence
  Description of Artificial Sequence: Synthetic polynucleotide
ucauugcaaagauugcugacuacagcauugcucaguacugcuguaauaugcuaaauguuacagcaguccugggcggugccguagucagcaaucuuugcaauga

SEQ ID NO: 768 - RNA, 103 nt, Artificial Sequence
  Description of Artificial Sequence: Synthetic polynucleotide
ucauugcaaagauugcugacuacagcauugcucaguacugcuguaauaugcuaaauguuacagcaguccuggguggugccguagucagcaaucuuugcaauga

SEQ ID NO: 769 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaggacugagcagugccguagugucgaaucuuugcaauga

SEQ ID NO: 770 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaggacugaguagugccguagugucgaaucuuugcaauga

SEQ ID NO: 771 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaggacugggcagugccguagugucgaaucuuugcaauga

SEQ ID NO: 772 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaggacuggguagugccguagugucgaaucuuugcaauga

SEQ ID NO: 773 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaggccugagcagugccguagugucgaaucuuugcaauga

SEQ ID NO: 774 - RNA, 103 nt, Artificial Sequence
  Description of Artificial Sequence: Synthetic polynucleotide
ucauugcaaagauugcugacuacagcauugcucaguacugcuguaauaugcuaaauguuaaagcaguacugagcagugccguagugucgaaucuuugcaauga

SEQ ID NO: 775 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacugagcagugccguagugucgaaucuuugcaauga

SEQ ID NO: 776 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacugaguagugccguagugucgaaucuuugcaauga

SEQ ID NO: 777 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacugggcagugccguagugucgaaucuuugcaauga

SEQ ID NO: 778 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggacuggguagugccguagugucgaaucuuugcaauga

SEQ ID NO: 779 - RNA, 103 nt, Artificial Sequence
  Description of Artificial Sequence: Synthetic polynucleotide
ucauugcaaagauugcugacuacagcauugcucaguacugcuguaauaugcuaaauguuacagcaggccugagcagugccguagugucgaaucuuugcaauga

SEQ ID NO: 780 - DNA, 45 bp, Artificial Sequence; identical to SEQ ID NO: 218
  Description of Artificial Sequence: Synthetic oligonucleotide

SEQ ID NO: 781 - DNA, 43 bp, Artificial Sequence; identical to SEQ ID NO: 219
  Description of Artificial Sequence: Synthetic oligonucleotide

SEQ ID NO: 782 - DNA, 43 bp, Artificial Sequence; identical to SEQ ID NO: 220
  Description of Artificial Sequence: Synthetic oligonucleotide

SEQ ID NO: 783 - DNA, 43 bp, Artificial Sequence; identical to SEQ ID NO: 221
  Description of Artificial Sequence: Synthetic oligonucleotide

SEQ ID NO: 784 - DNA, 45 bp, Artificial Sequence; identical to SEQ ID NO: 222
  Description of Artificial Sequence: Synthetic oligonucleotide

SEQ ID NO: 785 - DNA, 45 bp, Artificial Sequence; identical to SEQ ID NO: 223
  Description of Artificial Sequence: Synthetic oligonucleotide

SEQ ID NO: 786 - DNA, 45 bp, Artificial Sequence; identical to SEQ ID NO: 224
  Description of Artificial Sequence: Synthetic oligonucleotide

SEQ ID NO: 787 - DNA, 43 bp, Artificial Sequence; identical to SEQ ID NO: 225
  Description of Artificial Sequence: Synthetic oligonucleotide

SEQ ID NO: 788 - DNA, 45 bp, Artificial Sequence; identical to SEQ ID NO: 226
  Description of Artificial Sequence: Synthetic oligonucleotide

SEQ ID NO: 789 - DNA, 45 bp, Artificial Sequence; identical to SEQ ID NO: 227
  Description of Artificial Sequence: Synthetic oligonucleotide

SEQ ID NO: 790 - DNA, 43 bp, Artificial Sequence; identical to SEQ ID NO: 228
  Description of Artificial Sequence: Synthetic oligonucleotide

SEQ ID NO: 791 - DNA, 45 bp, Artificial Sequence; identical to SEQ ID NO: 229
  Description of Artificial Sequence: Synthetic oligonucleotide

SEQ ID NO: 792 - DNA, 45 bp, Artificial Sequence; identical to SEQ ID NO: 230
  Description of Artificial Sequence: Synthetic oligonucleotide

SEQ ID NO: 793 - DNA, 43 bp, Artificial Sequence; identical to SEQ ID NO: 231
  Description of Artificial Sequence: Synthetic oligonucleotide

SEQ ID NO: 794 - DNA, 45 bp, Artificial Sequence; identical to SEQ ID NO: 232
  Description of Artificial Sequence: Synthetic oligonucleotide

SEQ ID NO: 795 - DNA, 45 bp, Artificial Sequence; identical to SEQ ID NO: 233
  Description of Artificial Sequence: Synthetic oligonucleotide

SEQ ID NO: 796 - DNA, 45 bp, Artificial Sequence; identical to SEQ ID NO: 234
  Description of Artificial Sequence: Synthetic oligonucleotide

SEQ ID NO: 797 - DNA, 45 bp, Artificial Sequence; identical to SEQ ID NO: 235
  Description of Artificial Sequence: Synthetic oligonucleotide

SEQ ID NO: 798 - DNA, 45 bp, Artificial Sequence; identical to SEQ ID NO: 236
  Description of Artificial Sequence: Synthetic oligonucleotide

SEQ ID NO: 799 - DNA, 43 bp, Artificial Sequence; identical to SEQ ID NO: 237
  Description of Artificial Sequence: Synthetic oligonucleotide

SEQ ID NO: 800 - DNA, 45 bp, Artificial Sequence; identical to SEQ ID NO: 238
  Description of Artificial Sequence: Synthetic oligonucleotide

SEQ ID NO: 801 - DNA, 45 bp, Artificial Sequence; identical to SEQ ID NO: 239
  Description of Artificial Sequence: Synthetic oligonucleotide

SEQ ID NO: 802 - DNA, 45 bp, Artificial Sequence; identical to SEQ ID NO: 240
  Description of Artificial Sequence: Synthetic oligonucleotide

SEQ ID NO: 803 - DNA, 45 bp, Artificial Sequence; identical to SEQ ID NO: 241
  Description of Artificial Sequence: Synthetic oligonucleotide

SEQ ID NO: 804 - DNA, 45 bp, Artificial Sequence; identical to SEQ ID NO: 242
  Description of Artificial Sequence: Synthetic oligonucleotide

SEQ ID NO: 805 - DNA, 43 bp, Artificial Sequence; identical to SEQ ID NO: 243
  Description of Artificial Sequence: Synthetic oligonucleotide

SEQ ID NO: 806 - DNA, 45 bp, Artificial Sequence; identical to SEQ ID NO: 244
  Description of Artificial Sequence: Synthetic oligonucleotide

SEQ ID NO: 807 - DNA, 46 bp, Artificial Sequence; identical to SEQ ID NO: 245
  Description of Artificial Sequence: Synthetic oligonucleotide

SEQ ID NO: 808 - DNA, 45 bp, Artificial Sequence; identical to SEQ ID NO: 246
  Description of Artificial Sequence: Synthetic oligonucleotide

SEQ ID NO: 809 - DNA, 45 bp, Artificial Sequence; identical to SEQ ID NO: 247
  Description of Artificial Sequence: Synthetic oligonucleotide

SEQ ID NO: 810 - DNA, 45 bp, Artificial Sequence; identical to SEQ ID NO: 248
  Description of Artificial Sequence: Synthetic oligonucleotide

SEQ ID NO: 811 - DNA, 45 bp, Artificial Sequence; identical to SEQ ID NO: 249
  Description of Artificial Sequence: Synthetic oligonucleotide

SEQ ID NO: 812 - RNA, 103 nt, Artificial Sequence
  Description of Artificial Sequence: Synthetic polynucleotide
ucauugcaaagauugcugacuacagcauugcucaguacugcuguaauaugcuaaauguuacagcaguacugagcagugccguagugucgaaucuuugcaauga

(19) United States
(12) Patent Application Publication
(10) Pub. No.: US 2023/0399635 A1
(43) Pub. Date: Dec. 14, 2023

(54) RNA-Editing Compositions and Methods of Use

(71) Applicant: Shape Therapeutics Inc., Seattle, WA (US)

(72) Inventors: Yiannis SAVVA, Seattle, WA (US); Richard SULLIVAN, Seattle, WA (US); Brian BOOTH, West Roxbury, MA (US); Adrian BRIGGS, Seattle, WA (US); Debojit BOSE, Seattle, WA (US); Susan BYRNE, Lexington, MA (US); Stephen BURLEIGH, Seattle, WA (US)

(21) Appl. No.: 18/036,176

(22) PCT Filed: Nov. 10, 2021
(86) PCT No.: PCT/US2021/058799

§ 371 Date: May 10, 2023

Related U.S. Application Data
(60) Provisional application No. 63/112,452, filed on Nov. 11, 2020.
(60) Provisional application No. 63/119,754, filed on Dec. 1, 2020.
(60) Provisional application No. 63/153,070, filed on Feb. 24, 2021.
(60) Provisional application No. 63/178,159, filed on Apr. 22, 2021.
(60) Provisional application No. 63/193,373, filed on May 26, 2021.

(51) Int. Cl. C12N 15/10; C12N 15/86; C12N 9/22; C12N 15/113

(52) U.S. Cl. CPC C12N 15/102; C12N 15/86; C12N 9/22; C12N 15/1138; C12N 2320/50; C12N 2320/34; C12N 2750/14143

(57) ABSTRACT

Provided herein are engineered latent guide RNAs that bind target RNAs to form a guide-target RNA scaffold and are substrates for RNA editing entities, which chemically modify the base of a nucleotide of the target RNA. Also provided herein are compositions, vectors, and cells comprising the engineered latent guide RNAs disclosed herein and methods of use thereof.

### CROSS REFERENCE TO RELATED APPLICATIONS

This application claims priority under 35 U.S.C. § 119 from Provisional Application Ser. No. 63/112,452, filed Nov. 11, 2020, Provisional Application Ser. No. 63/119,754, filed Dec. 1, 2020, Provisional Application Ser. No. 63/153,070, filed Feb. 24, 2021, Provisional Application Ser. No. 63/178,159, filed Apr. 22, 2021, and Provisional Application Ser. No. 63/193,373, filed May 26, 2021, the disclosures of which are incorporated herein by reference in their entirety.

### SUMMARY

Disclosed herein are engineered guide RNAs. In some embodiments, an engineered guide RNA, upon hybridization to a target RNA implicated in a disease or condition, can form a guide-target RNA scaffold comprising a structural feature selected from the group consisting of a bulge, an internal loop, a hairpin, and any combination thereof. In some embodiments, the structural feature can substantially form upon hybridization to the target RNA. In some embodiments, an engineered guide RNA is configured to hybridize to a target RNA implicated in a disease or condition. In some embodiments, the guide-target RNA scaffold further comprises a mismatch. In some embodiments, the mismatch is an adenosine/cytosine (A/C) mismatch, wherein the adenosine (A) is present in the target RNA and the cytosine (C) is present in the engineered guide RNA. In some embodiments, the guide-target RNA scaffold comprises a wobble base pair. In some embodiments, the guide-target RNA scaffold can be a substrate for an RNA editing entity that chemically modifies a base of a nucleotide in the target RNA. In some embodiments, the RNA editing entity chemically modifies the adenosine in the target RNA to an inosine. In some embodiments, the guide-target RNA scaffold comprises a structured motif comprising two or more structural features selected from the group consisting of a bulge, an internal loop, a hairpin, and any combination thereof. In some embodiments, the guide-target RNA scaffold comprises at least two, three, four, five, six, seven, eight, nine, or 10 structural features selected from the group consisting of a bulge, an internal loop, a hairpin, and any combination thereof. In some embodiments, the structural feature is a bulge. In some embodiments, the bulge is an asymmetric bulge. In some embodiments, the bulge is a symmetric bulge. In some embodiments, the bulge comprises from 1 to 4 nucleotides of the engineered guide RNA and from 0 to 4 nucleotides of the target RNA. In some embodiments, the bulge comprises from 0 to 4 nucleotides of the engineered guide RNA and from 1 to 4 nucleotides of the target RNA. In some embodiments, the asymmetric bulge is an X₁/X₂ asymmetric bulge, wherein X₁ is the number of nucleotides of the target RNA in the asymmetric bulge and X₂ is the number of nucleotides of the engineered guide RNA in the asymmetric bulge, wherein the X₁/X₂ asymmetric bulge is a 0/1 asymmetric bulge, a 1/0 asymmetric bulge, a 0/2 asymmetric bulge, a 2/0 asymmetric bulge, a 0/3 asymmetric bulge, a 3/0 asymmetric bulge, a 0/4 asymmetric bulge, a 4/0 asymmetric bulge, a 1/2 asymmetric bulge, a 2/1 asymmetric bulge, a 1/3 asymmetric bulge, a 3/1 asymmetric bulge, a 1/4 asymmetric bulge, a 4/1 asymmetric bulge, a 2/3 asymmetric bulge, a 3/2 asymmetric bulge, a 2/4 asymmetric bulge, a 4/2 asymmetric bulge, a 3/4 asymmetric bulge, or a 4/3 asymmetric bulge. In some embodiments, the symmetric bulge is an X₁/X₂ symmetric bulge, wherein X₁ is the number of nucleotides of the target RNA in the symmetric bulge and X₂ is the number of nucleotides of the engineered guide RNA in the symmetric bulge, and wherein the X₁/X₂ symmetric bulge a 2/2 symmetric bulge, a 3/3 symmetric bulge, or a 4/4 symmetric bulge. In some embodiments, the structural feature comprises an internal loop. In some embodiments, the internal loop comprises an asymmetric internal loop. In some embodiments, the internal loop comprises a symmetric internal loop. In some embodiments, the asymmetric internal loop is an X₁/X₂ asymmetric internal loop, wherein X₁ is the number of nucleotides of the target RNA in the asymmetric internal loop and X₂ is the number of nucleotides of the engineered guide RNA in the asymmetric internal loop, and wherein the X₁/X₂ asymmetric internal loop is a 5/6 asymmetric internal loop, a 6/5 asymmetric internal loop, a 5/7 asymmetric internal loop, a 7/5 asymmetric internal loop, a 5/8 asymmetric internal loop, a 8/5 asymmetric internal loop, a 5/9 asymmetric internal loop, a 9/5 asymmetric internal loop, a 5/10 asymmetric internal loop, a 10/5 asymmetric internal loop, a 6/7 asymmetric internal loop, a 7/6 asymmetric internal loop, a 6/8 asymmetric internal loop, a 8/6 asymmetric internal loop, a 6/9 asymmetric internal loop, a 9/6 asymmetric internal loop, a 6/10 asymmetric internal loop, a 10/6 asymmetric internal loop, a 7/8 asymmetric internal loop, a 8/7 asymmetric internal loop, a 7/9 asymmetric internal loop, a 9/7 asymmetric internal loop, a 7/10 asymmetric internal loop, a 10/7 asymmetric internal loop, a 8/9 asymmetric internal loop, a 9/8 asymmetric internal loop, a 8/10 asymmetric internal loop, a 10/8 asymmetric internal loop, or a 9/10 asymmetric internal loop, or a 10/9 asymmetric internal loop. In some embodiments, the symmetric internal loop is an X₁/X₂ symmetric internal loop, wherein X₁ is the number of nucleotides of the target RNA in the symmetric internal loop and X₂ is the number of nucleotides of the engineered guide RNA in the symmetric internal loop, and wherein the X₁/X₂ symmetric internal loop is a 5/5 symmetric internal loop, a 6/6 symmetric internal loop, a 7/7 symmetric internal loop, a 8/8 symmetric internal loop, a 9/9 symmetric internal loop, a 10/10 symmetric internal loop, a 12/12 symmetric internal loop, a 15/15 symmetric internal loop, or a 20/20 symmetric internal loop. In some embodiments, the internal loop is formed by at least 5 nucleotides on either the engineered guide RNA or the target RNA. In some embodiments, the internal loop is formed by from 5 to 1000 nucleotides of either the engineered guide RNA or the target RNA. In some embodiments, the internal loop is formed by from 5 to 50 nucleotides of either the engineered guide RNA or the target RNA. In some embodiments, the internal loop is formed by from 5 to 20 nucleotides of either the engineered guide RNA or the target RNA. In some embodiments, the structural feature comprises a hairpin. In some embodiments, the hairpin comprises a non-recruitment hairpin. In some embodiments, a loop portion of the hairpin comprises from about 3 to about 15 nucleotides in length. In some embodiments, the engineered guide RNA further comprises at least two additional structural features that comprise at least two mismatches. In some embodiments, at least one of the at least two mismatches is a G/G mismatch. In some embodiments, the engineered guide RNA further comprises an additional structural feature that comprises a wobble base pair. In some embodiments, the wobble base pair comprises a guanine paired with a uracil. In some embodiments, the target RNA comprises a 5′ guanosine adjacent to the adenosine in the target RNA that is chemically modified to an inosine by the RNA editing entity. In some embodiments, the engineered guide RNA comprises a 5′ guanosine adjacent to the cytosine of the A/C mismatch. In some embodiments, the RNA editing entity is: (a) an adenosine deaminase acting on RNA (ADAR); (b) a catalytically active fragment of (a); (c) a fusion polypeptide comprising (a) or (b); or (d) any combination of these. In some embodiments, the RNA editing entity is endogenous to a cell. In some embodiments, the RNA editing entity comprises an ADAR. In some embodiments, the ADAR comprises human ADAR (hADAR). In some embodiments, the ADAR comprises ADAR1, ADAR2, ADAR3, or any combination thereof. In some embodiments, the ADAR1 comprises ADAR1p110, ADAR1p150, or a combination thereof. In some embodiments, the engineered guide RNA comprises a modified RNA base, an unmodified RNA base, or a combination thereof. In some embodiments, the target RNA is an mRNA molecule. In some embodiments, the target RNA is a pre-mRNA molecule. In some embodiments, the target RNA is APP, ABCA4, SERPINA1, HEXA, LRRK2, CFTR, SNCA, MAPT, or LIPA, a fragment any of these, or any combination thereof. In some embodiments, the target RNA encodes amyloid precursor polypeptide, ATP-binding cassette, sub-family A, member 4 (ABCA4) polypeptide, alpha-1 antitrypsin (AAT) polypeptide, hexosaminidase A enzyme, leucine-rich repeat kinase 2 (LRRK2) polypeptide, CFTR polypeptide, alpha synuclein polypeptide, Tau polypeptide, or lysosomal acid lipase polypeptide. In some embodiments, the target RNA encodes ABCA4 polypeptide. In some embodiments, the target RNA comprises a G to A substitution at position 5882, 6320, or 5714, relative to a wildtype ABCA4 gene sequence of accession number NC_000001.11:c94121149-93992837. In some embodiments, the guide-target RNA scaffold comprises one or more structural features selected from TABLE 7, TABLE, 9, TABLE 10, TABLE 11, TABLE 18, or TABLE 19. In some embodiments, the guide-target RNA scaffold comprises a structural features selected from the group consisting of: (i) one or more X₁/X₂ bulges, wherein X₁ is the number of nucleotides of the target RNA in the bulge and X₂ is the number of nucleotides of the engineered guide RNA in the bulge, and wherein the one or more bulges is a 2/1 asymmetric bulge, a 1/0 asymmetric bulge, a 2/2 symmetric bulge, a 3/3 symmetric bulge, or a 4/4 symmetric bulge; (ii) an X₁/X₂ internal loop, wherein X₁ is the number of nucleotides of the target RNA in the internal loop and X₂ is the number of nucleotides of the engineered guide RNA in the internal loop, and wherein the internal loop is a 5/5 symmetric loop (iii) one or more mismatches, wherein the one or more mismatches is a G/G mismatch, an A/C mismatch, or a G/A mismatch, (iv) a G/U wobble base pair or a U/G wobble base pair, and (v) any combination thereof. In some embodiments, the guide-target RNA scaffold comprises a 2/1 asymmetric bulge, a 1/0 asymmetric bulge, a G/G mismatch, an A/C mismatch, and a 3/3 symmetric bulge. In some embodiments, the engineered guide RNA has a length of from 80 to 175 nucleotides. In some embodiments, the engineered guide RNA comprises a polynucleotide having at least 80%, at least 85%, at least 90%, at least 95%, at least 97%, at least 99%, or 100% sequence identity to SEQ ID NO: 21, SEQ ID NO: 29, SEQ ID NO: 11, SEQ ID NO: 22, SEQ ID NO: 30, SEQ ID NO: 12, SEQ ID NO: 339-SEQ ID NO: 341, or SEQ ID NO: 292-SEQ ID NO: 296. In some embodiments, the engineered guide RNA comprises a polynucleotide at least 80%, at least 85%, at least 90%, at least 95%, at least 97%, at least 99%, or 100% sequence identity to any one of SEQ ID NO: 11-34, 58, 218-289, 291-296, or 328-343. In some embodiments, the target RNA encodes LRRK2 polypeptide. In some embodiments, the LRRK2 polypeptide comprises a mutation selected from the group consisting of: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, or Q2490NfsX3. In some embodiments, the guide-target RNA scaffold comprises one or more structural features selected from TABLE 12, TABLE 15, TABLE 25, TABLE 26, TABLE 27, TABLE 17, or TABLE 20. In some embodiments, the guide-target RNA scaffold comprises one or more structural features selected from the group consisting of: (i) one or more X₁/X₂ bulges, wherein X₁ is the number of nucleotides of the target RNA in the bulge and X₂ is the number of nucleotides of the engineered guide RNA in the bulge, and wherein the one or more bulges is a 0/1 asymmetric bulge, a 2/2 symmetric bulge, a 3/3 symmetric bulge, or a 4/4 symmetric bulge; (ii) one or more X₁/X₂ internal loops, wherein X₁ is the number of nucleotides of the target RNA in the internal loop and X₂ is the number of nucleotides of the engineered guide RNA in the internal loop, and wherein the one or more internal loops is a 5/0 asymmetric internal loop, a 5/4 asymmetric internal loop, a 5/5 symmetric internal loop, a 6/6 symmetric internal loop, a 7/7 symmetric internal loop, or a 10/10 symmetric internal loop; (iii) one or more mismatches, wherein the one or more mismatches is an A/C mismatch, an A/G mismatch, a C/U mismatch, a G/A mismatch, or a C/C mismatch, (iv) a G/U wobble base pair or a U/G wobble base pair, and (v) any combination thereof. In some embodiments, the guide-target RNA scaffold comprises a 6/6 symmetrical internal loop, an A/C mismatch, an A/G mismatch, and a C/U mismatch. In some embodiments, the engineered guide RNA has a length of from 80 to 175 nucleotides. In some embodiments, the engineered guide RNA comprises a polynucleotide having at least 80%, at least 85%, at least 90%, at least 95%, at least 97%, at least 99%, or 100% sequence identity to SEQ ID NO: 30, SEQ ID NO: 344, or SEQ ID NO: 345. In some embodiments, the engineered guide RNA comprises a polynucleotide having at least 80%, at least 85%, at least 90%, at least 95%, at least 97%, at least 99%, or 100% sequence identity to any one of SEQ ID NO: 35-42, 46-52, 111-207, or 344-345. In some embodiments, the target RNA encodes SNCA polypeptide. In some embodiments, the engineered guide RNA hybridizes to a sequence of the target RNA selected from the group consisting of: a 5′ untranslated region (UTR), a 3′ UTR, and a translation initiation site of an SNCA gene. In some embodiments, the guide-target RNA scaffold comprises one or more structural features selected from TABLE 21, TABLE 23, or TABLE 28. In some embodiments, the guide-target RNA scaffold comprises one or more structural features selected from the group consisting of: (i) an X₁/X₂ bulge, wherein X₁ is the number of nucleotides of the target RNA in the bulge and X₂ is the number of nucleotides of the engineered guide RNA in the bulge, and wherein the bulge is a 4/4 symmetric bulge; (ii) one or more X₁/X₂ internal loops, wherein X₁ is the number of nucleotides of the target RNA in the internal loop and X₂ is the number of nucleotides of the engineered guide RNA in the internal loop, and wherein the one or more internal loop is a 5/5 symmetric loop, an 8/8 symmetric loop, or a 49/4 asymmetric loop; (iii) one or more mismatches, wherein the one or more mismatches is an A/C mismatch, a G/G mismatch, a G/A mismatch, a U/C mismatch, or an A/A mismatch, (iv) any combination thereof. In some embodiments, the engineered guide RNA has a length of from 80 to 175 nucleotides. In some embodiments, the engineered guide RNA comprises a polynucleotide having at least 80%, at least 85%, at least 90%, at least 95%, at least 97%, at least 99%, or 100% sequence identity to any one of SEQ ID NO: 59-101, 104-108, and 208-217. In some embodiments, the target RNA encodes SERPINA1. In some embodiments, the target RNA comprises a G to A substitution at position 9989, relative to a wildtype SERPINA1 gene sequence of accession number NC_000014.9:c94390654-94376747. In some embodiments, the guide-target RNA scaffold comprises one or more structural features selected from TABLE 5, TABLE 29, TABLE 30, TABLE 31, TABLE 32, TABLE 33, TABLE 34, TABLE 35, or TABLE 36. In some embodiments, the guide-target RNA scaffold comprises one or more structural features selected from the group consisting of: (i) one or more X₁/X₂ bulges, wherein X₁ is the number of nucleotides of the target RNA in the bulge and X₂ is the number of nucleotides of the engineered guide RNA in the bulge, and wherein the bulge is a 0/2 asymmetric bulge, a 0/3 asymmetric bulge, a 1/0 asymmetric bulge, a 2/0 asymmetric bulge, a 2/2 symmetric bulge, a 3/0 asymmetric bulge, a 2/2 symmetric bulge, or a 3/3 symmetric bulge; (ii) an X₁/X₂ internal loop, wherein X₁ is the number of nucleotides of the target RNA in the internal loop and X₂ is the number of nucleotides of the engineered guide RNA in the internal loop, and wherein the internal loop is a 5/5 symmetric internal loop; (iii) one or more mismatches, wherein the one or more mismatches is an A/C mismatch, an A/A mismatch, and a G/A mismatch, (iv) a G/U wobble base pair, or a U/G wobble base pair; and (v) any combination thereof. In some embodiments, the engineered guide RNA has a length of from 80 to 175 nucleotides. In some embodiments, the engineered guide RNA comprises a polynucleotide having at least 80%, at least 85%, at least 90%, at least 95%, at least 97%, at least 99%, or 100% sequence identity to any one of SEQ ID NO: 6-10, 102-103 or 297-327. In some embodiments, the base of the nucleotide of the target RNA that is modified by the RNA editing entity is comprised in a point mutation of the target RNA. In some embodiments, the point mutation comprises a missense mutation. In some embodiments, the point mutation is a nonsense mutation. In some embodiments, the nonsense mutation is a premature UAA stop codon. In some embodiments, the structural feature increases selectivity of editing a target adenosine in the target RNA relative to an otherwise comparable guide RNA lacking the structural feature. In some embodiments, the structural feature decreases an amount of RNA editing of local off-target adenosines within 200, within 100, within 50, within 25, within 10, within 5, within 2, or 1 within 1 nucleotide 5′ or 3′ of a target adenosine in the target RNA by the RNA editing entity, relative to an otherwise comparable guide RNA lacking the structural feature.

Also disclosed herein are engineered RNAs comprising (a) an engineered guide RNA as described herein, and (b) a U7 snRNA hairpin sequence, a SmOPT sequence, or a combination thereof. In some embodiments, the U7 hairpin has a sequence of TAGGCTTTCTGGCTTTTTACCGGAAAGCCCCT (SEQ ID NO: 389) or CAGGTTTTCTGACTTCGGTCGGAAAACCCCT (SEQ ID NO: 394). In some embodiments, the SmOPT sequence has a sequence of AATTTTTGGAG (SEQ ID NO: 390).

Also disclosed herein are polynucleotides encoding an engineered guide RNA as described herein or an engineered RNA as described herein.

Also disclosed herein are delivery vectors comprising an engineered guide RNA as described herein, an engineered RNA as described herein, or a polynucleotide as described herein (encoding an engineered guide RNA or an engineered RNA). In some embodiments, the delivery vector is a viral vector. In some embodiments, the viral vector is an adeno-associated viral (AAV) vector or a derivative thereof. In some embodiments, the AAV vector is from an adeno-associated virus having a serotype selected from AAV1, AAV2, AAV3, AAV4, AAV5, AAV6, AAV7, AAV8, AAV9, AAV10, AAV 11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some embodiments, the AAV vector is a recombinant AAV (rAAV) vector, a hybrid AAV vector, a chimeric AAV vector, a self-complementary AAV (scAAV) vector, a single-stranded AAV or any combination thereof. In some embodiments, the AAV vector comprises a genome comprising a replication gene and inverted terminal repeats from a first AAV serotype and a capsid protein from a second AAV serotype. In some embodiments, the AAV vector is an AAV 2/5 vector, an AAV 2/6 vector, an AAV 2/7 vector, an AAV2/8 vector, or an AAV 2/9 vector. In some embodiments, the inverted terminal repeats comprise a 5′ inverted terminal repeat, a 3′ inverted terminal repeat, and a mutated inverted terminal repeat. In some embodiments, the mutated inverted terminal repeat lacks a terminal resolution site.

Also disclosed herein are pharmaceutical compositions comprising: (a) an engineered guide RNA as described herein, an engineered RNA as described herein, a polynucleotide as described herein, or a delivery vector as described herein, and (b) a pharmaceutically acceptable: excipient, carrier, or diluent. In some embodiments, the pharmaceutical composition is in unit dose form. In some embodiments, the pharmaceutical composition further comprises an additional therapeutic agent. In some embodiments, the additional therapeutic agent comprises an ammonia reducer, a beta blocker, a synthetic hormone, an antibiotic, or an antiviral drug, a vascular endothelial growth factor (VEGF) inhibitor, a stem cell treatment, a vitamin or modified form thereof, or any combination thereof.

Also disclosed herein are methods of editing a target RNA in a cell. In some embodiments, the method comprises: administering to the cell an effective amount of an engineered guide RNA as described herein, an engineered RNA as described herein, a polynucleotide as described herein, a delivery vector as described herein, or a pharmaceutical composition as described herein.

Also disclosed herein are methods of treating a disease in a subject. In some embodiments, the method comprises administering to the subject an effective amount of an engineered guide RNA as described herein, an engineered RNA as described herein, a polynucleotide as described herein, a delivery vector as described herein, or a pharmaceutical composition as described herein. In some embodiments, the engineered guide RNA is administered as a unit dose. In some embodiments, the unit dose is an amount sufficient to treat the subject. In some embodiments, the administering is intrathecal, intraocular, intravitreal, retinal, intravenous, intramuscular, intraventricular, intracerebral, intracerebellar, intracerebroventricular, intraperenchymal, subcutaneous, or a combination thereof. In some embodiments, the disease comprises a neurological disease. In some embodiments, the neurological disease comprises Parkinson's disease, Alzheimer's disease, a Tauopathy, or dementia. In some embodiments, the neurological disease is associated with elevated levels of SNCA polypeptide, relative to a healthy subject that does not have the neurological disease or condition. In some embodiments, the engineered guide RNA hybridizes to a sequence of a target RNA encoding the SNCA polypeptide selected from the group consisting of: a 5′ untranslated region (UTR), a 3′ UTR, and a translation initiation site of SNCA; wherein hybridization produces a guide-target RNA scaffold that is a substrate for an RNA editing entity that chemically modifies a base of a nucleotide in the sequence of the target RNA, thereby reducing levels of the SNCA polypeptide. In some embodiments, the engineered guide RNA hybridizes to a sequence of a target RNA encoding the translation initiation site of SNCA. In some embodiments, the engineered guide RNA comprises a polynucleotide having at least 80%, at least 85%, at least 90%, at least 95%, at least 97%, at least 99%, or 100% sequence identity to any one of SEQ ID NO: 59-101, 104-108, and 208-217. In some embodiments, the engineered guide RNA comprises has a percent on-target editing for ADAR2 of at least about 90%. In some embodiments, the neurological disease is associated with a mutation of an LRRK2 polypeptide encoded by the target RNA, wherein the mutation is selected from the group consisting of: E10L, A30P, S52F, E46K, A53T, L119P, A211V, C228S, E334K, N363S, V366M, A419V, R506Q, N544E, N551K, A716V, M712V, 1723V, P755L, R793M, 1810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, or Q2490NfsX3. In some embodiments, the neurological disease is associated with a mutation of an LRRK2 polypeptide encoded by the target RNA, wherein the mutation is a G2019S mutation. In some embodiments, the engineered guide RNA comprises a polynucleotide having at least 80%, at least 85%, at least 90%, at least 95%, at least 97%, at least 99%, or 100% sequence identity to any one of SEQ ID NO: 35-42, 46-52, 111-207, or 344-345. In some embodiments, the engineered guide RNA comprises has a percent on-target editing for ADAR1 of at least about 60% or a percent on-target editing for ADAR2 of at least about 90%. In some embodiments, the disease comprises a liver disease. In some embodiments, the liver disease comprises liver cirrhosis. In some embodiments, the liver disease is alpha-1 antitrypsin (AAT) deficiency. In some embodiments, the AAT deficiency is associated with a G to A substitution at position 9989 of a wildtype SERPINA1 gene sequence of accession number NC_000014.9:c94390654-94376747. In some embodiments, the engineered latent wherein the engineered guide RNA comprises a polynucleotide having at least 80%, at least 85%, at least 90%, at least 95%, at least 97%, at least 99%, or 100% sequence identity to any one of SEQ ID NO: 6-10, 102-103 or 297-327. In some embodiments, the engineered guide RNA comprises has a percent on-target editing for ADAR1 of at least about 60% or a percent on-target editing for ADAR2 of at least about 90%. In some embodiments, the disease is a macular degeneration. In some embodiments, the macular degeneration is Stargardt Disease. In some embodiments, the Stargardt disease is associated with a G to A substitution at position 5882, 6320, or 5714 of a wildtype ABCA4 gene sequence of accession number NC_000001.11:c94121149-93992837. In some embodiments, the Stargardt disease is associated with a G to A substitution at position 5882. In some embodiments, the engineered guide RNA comprises a polynucleotide having at least 80%, at least 85%, at least 90%, at least 95%, at least 97%, at least 99%, or 100% sequence identity to any one of SEQ ID NO: 11-34, 58, 218-289, 291-296, or 328-343. In some embodiments, the engineered guide RNA comprises has a percent on-target editing for ADAR1 of at least about 70% or a percent on-target editing for ADAR2 of at least about 80%. In some embodiments, the subject is diagnosed with the disease or the condition.

Also disclosed herein is an engineered guide RNA as described herein, an engineered RNA as described herein, a polynucleotide as described herein, a delivery vector as described herein, or a pharmaceutical composition as described herein, for use as a medicament.

Also disclosed herein is an engineered guide RNA as described herein, an engineered RNA as described herein, a polynucleotide as described herein, a delivery vector as described herein, or a pharmaceutical composition as described herein, for use in treatment of a neurological disease. In some embodiments, the neurological disease is Parkinson's disease, Alzheimer's disease, a Tauopathy, or dementia.

Also disclosed herein is an engineered guide RNA as described herein, an engineered RNA as described herein, a polynucleotide as described herein, a delivery vector as described herein, or a pharmaceutical composition as described herein, for use in treatment of a liver disease. In some embodiments, the liver disease comprises liver cirrhosis. In some embodiments, the liver disease is alpha-1 antitrypsin (AAT) deficiency.

Also disclosed herein is an engineered guide RNA as described herein, an engineered RNA as described herein, a polynucleotide as described herein, a delivery vector as described herein, or a pharmaceutical composition as described herein, for use in treatment of macular degeneration. In some embodiments, the macular degeneration is Stargardt disease.

Also disclosed herein is the use of an engineered guide RNA as described herein, an engineered RNA as described herein, a polynucleotide as described herein, a delivery vector as described herein, or a pharmaceutical composition as described herein, for the manufacture of a medicament.

Also disclosed herein is the use of an engineered guide RNA as described herein, an engineered RNA as described herein, a polynucleotide as described herein, a delivery vector as described herein, or a pharmaceutical composition as described herein, for the manufacture of a medicament for the treatment of a neurological disease, a liver disease or macular degeneration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of embodiments of the present disclosure will be obtained by reference to the following detailed description that sets forth illustrative embodiments, and the accompanying drawings of which:

FIG. 1 shows an example of a workflow according to the methods described herein.

FIGS. 2A and 2B are images illustrating use of an engineered guide disclosed herein to target a pre-mRNA molecule (FIG. 2A) and a mature mRNA molecule (FIG. 2B).

FIG. 3 illustrates an example of a drosophila ADAR substrate from the Shaker gene capable of facilitating RNA editing of the target A (indicated by an arrow) within a 5′ G context.

FIG. 4 provides the nucleotide sequences of the RNA molecules forming the drosophila ADAR substrate of FIG. 3 and annotations detailing the structural features formed by nucleotide interactions.

FIGS. 5A-5B shows double-stranded substrates described herein formed by engineered guides disclosed herein and a target RNA molecule encoded by the ABCA4 gene. FIG. 5A shows an engineered guide exhibiting full complementarity to the target RNA molecule. FIG. 5B shows an engineered guide exhibiting partial complementarity to the target RNA molecule and forming a double stranded substrate that exhibits full mimicry of the naturally occurring drosophila substrate shown in FIGS. 3 and 4.

FIGS. 6A-6B show nucleotide sequences of engineered guides disclosed herein with annotations detailing the structural features formed by nucleotide interactions. FIG. 6A shows an exemplary engineered guide exhibiting partial complementarity to the target RNA molecule and forming a double stranded substrate that exhibits full mimicry of the naturally occurring drosophila substrate shown in FIG. 4. FIG. 6B shows a table comparing the locations of the structural features depicted in FIGS. 6A and 4, as well as the changes in nucleotide sequence between the guide of FIG. 6A and a guide having full complementarity to the target RNA molecule.

FIG. 7 shows an exemplary engineered guide exhibiting partial complementarity to the target RNA molecule and forming a double stranded substrate that exhibits full mimicry of the naturally occurring drosophila substrate shown in FIG. 6B.

FIG. 8 shows double stranded substrates formed by engineered guides disclosed herein and target RNA molecules disclosed herein exhibiting varying levels of mimicry of the naturally occurring substrates depicted in FIG. 4.

FIGS. 9A-9F show double-stranded substrates formed by engineered guides disclosed herein and target molecules disclosed herein. The engineered guides can be 100 nucleotides in length comprising, at nucleotide 80, plus or minus 2 nucleotides, from the 5′ end, a cytosine intended for pairing with the adenine to be edited by an ADAR, referred to as “100.80” guides herein. For example, “100.80” refers to a guide in which the cytosine intended for pairing with the adenine to be edited can be at nucleotide 82 from the 5′ end. The double stranded substrates exhibit varying levels of mimicry of the naturally occurring drosophila ADAR substrate.

FIGS. 10A-10H show double-stranded substrates formed by engineered guides disclosed herein and target molecules disclosed herein. The engineered guides can be 150 nucleotides in length comprising, at nucleotide 125, plus or minus 2 nucleotides, from the 5′ end, a cytosine intended for pairing with the adenine to be edited by an ADAR, referred to as “150.125” guides herein. For example, “150.125” refers to a guide in which the cytosine intended for pairing with the adenine to be edited can be at nucleotide 123 from the 5′ end. The double stranded substrates exhibit varying levels of mimicry of the naturally occurring drosophila ADAR substrate.

FIGS. 11A-11J show double-stranded substrates formed by engineered guides disclosed herein and target molecules disclosed herein. The engineered guides can be 150 nucleotides in length comprising, at nucleotide 75, plus or minus 2 nucleotides, from the 5′ end, a cytosine intended for pairing with the adenine to be edited by an ADAR, referred to as “150.75” guides herein. For example, “150.75” refers to a guide in which the cytosine intended for pairing with the adenine to be edited can be at nucleotide 77 from the 5′ end. The double stranded substrates exhibit varying levels of mimicry of the naturally occurring drosophila ADAR substrate.

FIGS. 12A and 12B illustrate double-stranded substrates formed by engineered guides disclosed herein and target sequences disclosed herein. FIG. 12A shows an engineered guide exhibiting full complementarity to the target RNA molecule. FIG. 12B shows an engineered guide exhibiting partial complementarity to the target RNA molecule and forming a double stranded substrate that exhibits full mimicry of a naturally occurring drosophila substrate.

FIG. 13 shows the percent editing of target RNA sequences effected by various engineered guides disclosed herein.

FIG. 14 shows the results of fold change luciferase assays performed to analyze the guide length and mismatch placement best suited to accommodate the guide patterns found in the drosophila ADAR substrate in engineered guides targeting mutations in RNA encoded by ABCA4.

FIG. 15 shows a plot of length versus mismatch placement for the 100.80, 150, 125, and 150.75 engineered guides disclosed herein.

FIG. 16 shows the experimental workflow used to assess the ability of engineered guides disclosed herein to correct c.5882G>A mutations expressed in ABCA4 miniaturized genes (mini-genes).

FIG. 17 shows a western blot of ADAR1, ADAR2, and GAPDH in the HEK293 cells generated by carrying out the experimental workflow depicted in FIG. 20. Cells used in experiments are in Lane 3 and expressed ADAR1 and ADAR2.

FIG. 18 shows the percent editing of TAG positive controls only, as determined by Sanger Sequencing in the experiment illustrated in FIG. 20.

FIG. 19 shows the percent editing of the c.5882 mutation in the ABCA4 minigene achieved by the three guides comprising varying degrees of structural mimicry to the drosophila ADAR substrate, as determined in the experiment illustrated in FIG. 18.

FIG. 20 shows a comparison of the % RNA editing achieved by the three engineered guides, comparing versions of the guides comprising no structural mimicry to the drosophila substrate to versions exhibiting complete structural mimicry to the drosophila substrate.

FIG. 21 shows an example of the Sanger sequencing reads of the target RNA after transfections with 150.125 guide comprising varying degrees of mimicry to the drosophila ADAR substrate.

FIG. 22 shows a gel electrophoresis image of the in vitro transcribed (IVT) templates for various anti-LRRK2 guide RNAs, as amplified by Q5 PCR. The primers listed in TABLE 14 were used for the amplification. Wt 0.100.50 is LRRK2_0.0.100.50 (no GluR2 domain, guide is 100 nucleotides in length, A to be edited in the target LRRK2 RNA is positioned at nucleotide 50 of the guide), intGluR2 is LRRK2_IntGluR2, flip_intGluR2 is LRRK2_FlipIntGluR2, Nat guided is LRRK2 Natguide, EIE is LRRK2_EIE, Wt 1.100.50 is LRRK2_1.1.100.50, and Wt 2.100.50 is LRRK2_2.2.100.50. The lane on the far left-hand side is the molecular marker.

FIG. 23 shows gel electrophoresis image of various purified IVT-produced anti-LRRK2 guide RNAs. 25 nmol of RNA was loaded in each lane. Wt 0.100.50 is LRRK2_0.0.100.50, intGluR2 is LRRK2_IntGluR2, flip_intGluR2 is LRRK2_FlipIntGluR2, Nature guided is LRRK2 Natguide, EIE is LRRK2_EIE, Wt 1.100.50 is LRRK2_1.1.100.50, and Wt 2.100.50 is LRRK2_2.2.100.50. The lane on the far left-hand side is the molecular marker. Some guide RNA sequences are shown in TABLE 12.

FIG. 24 shows Sanger sequencing traces of the 6,055th nucleotide in the LRRK2 G2019S heterozygote cells treated with different anti-LRRK2 guide RNAs and controls. The cells were contracted with the guide RNAs for 3 hours (left panel) or 7 hours (right panel). The cells were EBV transformed B cells heterozygous for the G2019S mutation. The cells were treated with different guide RNAs. The RNA editing efficiency was calculated by the difference of the trace signal of the LRRK2 mRNA with a G (edited) and an A (unedited). The trace signal was measured by Sanger sequencing. By 3 hours (left panel), the RNA editing efficiency of LRRK2 FlipIntGluR2 (labeled as IntFlip) reached ˜14%, as opposed to 0% in Control (Ctrl). By 7 hours (right panel), other guide RNAs, such as LRRK2_0.100.50 (labeled as 0.100.50) and LRRK2_1.100.50 (labeled as 1.100.50), also showed ˜12% and 13.5% editing, respectively.

FIG. 25A show a non-limiting example of a double-stranded substrate formed by an engineered guide.

FIG. 25B show a non-limiting example of a double stranded substrate mimic.

FIG. 26 show a non-limiting example of a double stranded substrate mimic.

FIG. 27 show a non-limiting example of a double stranded substrate mimic.

FIG. 28 show a non-limiting example of a double stranded substrate mimic.

FIG. 29A shows the target nucleotide editing frequency of various positions of a LRRK2 target RNA using the perfect duplex (fully complementary to the target motif) guide RNA design or the A-C mismatch guide design and ADAR2. The Y-axis shows the percent editing frequency of various positions of the target RNA. The X-axis shows various positions of the target RNA. The arrow indicates the target nucleotide A. The top panel shows the target nucleotide editing frequency of a perfect duplex (fully complementary to the target motif) guide RNA with the target RNA. The bottom panel shows the target nucleotide editing frequency of a A-C mismatch guide RNA at the target A in the target RNA. The on-target target nucleotide editing is less than about 20% for either guide RNAs.

FIG. 29B shows a summary of the kinetic rates of target nucleotide editing in a high throughput guide screening assay performed on a target RNA LRRK2 and ADAR2 using 2540 guide RNA sequences. The X-axis shows the position of a base on the target RNA relative to the edit site. Position 0 is the target nucleotide. The number on the right of the target nucleotide indicates the nucleotides downstream of the target nucleotide. The number on the left of the target nucleotide indicates the nucleotides upstream of the target nucleotide. The Y-axis lists the guide RNAs tested. The color bar indicates the frequency of the editing; a lighter color indicates more editing while a darker color indicates less editing. Each position summarizes the frequencies of editing for all the time points in which the frequencies of editing were measured. The on-target and off-target target A are labelled.

FIG. 29C shows the target nucleotide editing frequency of various positions of a LRRK2 target RNA using a top-ranked engineered design identified in FIG. 38B and ADAR2. The Y-axis shows the percent editing frequency of various positions of the target RNA. The X-axis shows various positions of the target RNA. The arrow indicates the target nucleotide A. The on-target target nucleotide editing is more than 80%

FIG. 30A shows the target nucleotide editing frequency of various positions of an ABCA4 target RNA using the V1 guide RNA design and ADAR1. The Y-axis shows the percent editing frequency of various positions of the target RNA. The X-axis shows various positions of the target RNA. The arrow indicates the target nucleotide A. The top panel shows the target nucleotide editing frequency of a V1 guide RNA with a perfect duplex (fully complementary to the target motif) with the target RNA. The bottom panel shows the target nucleotide editing frequency of a V1 guide RNA with a A-C mismatch at the target A. None of the two guide RNAs provided any base editing at the target nucleotide A.

FIG. 30B shows a summary of the frequency of target nucleotide editing in a high throughput guide screening assay performed on a target RNA ABCA4 and ADAR1 using 2500 guide RNA sequences. The X-axis shows the position of a base on the target RNA relative to the edit site. Position 0 is the target nucleotide. The number on the right of the target nucleotide indicates the nucleotides downstream of the target nucleotide. The number on the left of the target nucleotide indicates the nucleotides upstream of the target nucleotide. The Y-axis lists the guide RNAs tested. The color bar indicates the frequency of the editing; a lighter color indicates more editing while a darker color indicates less editing. Each position summarizes the frequencies of editing for all the time points in which the frequencies of editing were measured. The on-target and off-target target A are labelled.

FIG. 30C shows the target nucleotide editing frequency of various positions of an ABCA4 target RNA using a top-ranked engineered design identified in FIG. 30B and ADAR1. The Y-axis shows the percent editing frequency of various positions of the target RNA. The X-axis shows various positions of the target RNA. The arrow indicates the target nucleotide A. The on-target target nucleotide editing is more than about 80%.

FIG. 31 shows the result of a high throughput guide screening assay performed on target RNAs LRRK2, ABCA4, and SERPINA1 with ADAR2. The X-axis shows the position of a base on the target RNA relative to the edit site. Position 0 is the target nucleotide. The number on the right of the target nucleotide indicates the nucleotides downstream of the target nucleotide. The number on the left of the target nucleotide indicates the nucleotides upstream of the target nucleotide. The time points (0, 20 seconds, 1 minute, 3 minutes, 10 minutes, 30 minutes, and 100 minutes) at which the editing frequency were measured is labeled on the top of the plot. The Y-axis lists the guide RNAs tested. The color bar indicates the kinetics of the editing; a lighter color indicates faster kinetics while a darker color indicates slower kinetics. The number of guide RNAs screened is labeled on the right of the plot.

FIG. 32 shows the result of a comparison of ADAR1 versus ADAR2-mediated editing in a high throughput guide screening assay performed on target RNAs LRRK2, ABCA4, and SERPINA1. The X-axis shows the position of a base on the target RNA relative to the edit site. Position 0 is the target nucleotide. The number on the right of the target nucleotide indicates the nucleotides downstream of the target nucleotide. The number on the left of the target nucleotide indicates the nucleotides upstream of the target nucleotide. The time point (0, 1 minute, 10 minutes, and 100 minutes) at which the editing frequency was measured is labeled on the top of the plot. For the time point 1 minute, 10 minutes, and 100 minutes, the editing kinetics of the editing mediated by ADAR1 vs ADAR2 are also shown. The Y-axis lists the guide RNAs tested. The color bar indicates the kinetics of the editing; a lighter color indicates faster kinetics while a darker color indicates slower kinetics.

FIG. 33 shows a summary of the analysis of selecting top candidate guide RNAs from a high throughput guide screening assay.

FIG. 34A shows the analysis for determining a target nucleotide editing rate in a high throughput guide screening assay performed on a target RNA LRRK2. The X-axis shows the position of a base on the target RNA relative to the edit site. Position 0 is the target nucleotide. The number on the right of the target nucleotide indicates the nucleotides downstream of the target nucleotide. The number on the left of the target nucleotide indicates the nucleotides upstream of the target nucleotide. The Y-axis lists the guide RNAs tested. The color bar indicates the kinetics of the editing; a lighter color indicates faster kinetics while a darker color indicates slower kinetics. Each position summarizes the kinetics of editing for all the time points in which the kinetics of editing were measured. The large box shown is an example from the different time points taken, which were 10^{-0.5} minutes, 10⁰ minutes, 10^{0.5} minutes, 10¹ minutes, 10^{1.5} minutes and 10² minutes.

FIG. 34B shows the editing kinetics of different guide RNAs on a LRRK2 target RNA. The percent editing of the target gene is indicated on the Y-axis and the time is shown on the X-axis. Three examples of guide RNAs are shown: a guide RNA with a perfect duplex (fully complementary to the target motif), a guide RNA with a single A-C mismatch, and a top-ranked engineered guide RNA. The top ranked guide RNA had higher percent editing in a shorter amount of time compared to the other guide RNA designs.

FIG. 35 shows ADAR1 and ADAR2 editing profiles with an engineered guide RNA. The percent editing of a target RNA ABCA4 is indicated on the Y-axis and the target region is shown on the X-axis. The gRNA shows +1 off-target editing with ADAR2 but not with ADAR1.

FIG. 36 shows the editing kinetics of different guide RNAs on a LRRK2 target RNA. The percent editing of the target gene is indicated on the Y-axis and the time is shown on the X-axis. Three examples of guide RNAs are shown: a top-ranked engineered guide RNA, a guide RNA with a single A-C mismatch, and a guide RNA with a perfect duplex (fully complementary to the target motif). The top ranked guide RNA had 30-fold increase in K_{obs} compared to other guide RNA designs.

FIG. 37 shows Venn diagrams summarizing the number of guide RNAs that provided on-target nucleotide editing at the target nucleotide of LRRK2 when using ADAR2 (a, >80% on target editing at the 100 min time point; b, <40% off-target editing at the 100 min time point; c, sequencing read depth: >50); the number of guide RNAs that provided on-targeting when using ADAR1 (a, >55% on target editing at the 100 min time point; b, <20% off-target editing at the 100 min time point; c, sequencing read depth: >50); or the top 20 guide RNAs for editing enzymatic kinetic using ADAR2 (a, >80% on target editing at the 100 min time point; b, enzymatic kinetic curve fit r²>0.8). 47 guide RNAs provided on-target nucleotide editing when using ADAR2. 71 guide RNAs provided on-target nucleotide editing when using ADAR1. 14 guide RNAs provided on-target nucleotide editing when using both ADAR1 and ADAR2. The number of guide RNAs that either provided on-target nucleotide editing when using either ADAR1 or ADAR2; or are the top 20 guide RNAs for editing enzymatic kinetic when using ADAR2 is 32.

FIG. 38A shows a summary of the kinetic rates of target nucleotide editing in a high throughput guide screening assay performed on a target RNA ABCA4 and ADAR2 at the 100 min time point. The X-axis shows the position of a base on the target RNA relative to the edit site. Position 0 is the target nucleotide. The number on the right of the target nucleotide indicates the nucleotides downstream of the target nucleotide. The number on the left of the target nucleotide indicates the nucleotides upstream of the target nucleotide. The Y-axis lists the guide RNAs tested. The color indicates the kinetics of the editing; a lighter color indicates faster kinetics while a darker color indicates slower kinetics.

FIG. 38B shows the editing kinetics of two optimized high-ranked engineered design guide RNAs on an ABCA4 target RNA. At the 100 min point, both guide RNAs (top two plots) showed about 80% on-target editing frequency of the target nucleotide A. For a comparison, the bottom plot shows a V1 design guide RNA with a A-C mismatch at the target nucleotide A. The target nucleotide editing frequency of various positions of the target RNA, when using ADAR1 or ADAR2, is shown on the right of the plot. The target A has a G on its 5′ side, the result demonstrates that an endogenous ADAR can be made to edit a 5′G site with the right guide RNA sequence.

FIG. 39 shows Venn diagrams summarizing the number of guide RNAs that provided on-target nucleotide editing at the target nucleotide of ABCA4 when using ADAR2 (a, >80% on target editing at the 100 min time point; b, <40% off-target editing at the 100 min time point; c, sequencing read depth: >50); the number of guide RNAs that provided on-target editing when using ADAR1 (a, >55% on target editing at the 100 min time point; b, <20% off-target editing at the 100 min time point; c, sequencing read depth: >50); or the top 20 guide RNAs for editing enzymatic kinetic when using ADAR2 (a, >80% on target editing at the 100 min time point; b, enzymatic kinetic curve fit r²>0.8). 33 guide RNAs provided on-target editing when using ADAR2. 153 guide RNAs provided on-target editing when using ADAR1. 24 guide RNAs provided on-target editing when using ADAR1 and ADAR2. The number of guide RNAs that either provided on-target editing when using ADAR1 and ADAR2; or are the top 20 guide RNAs for editing enzymatic kinetic when using ADAR2 is 32. The number of guide RNAs that provided on-target editing when using ADAR1 and ADAR2; and are the top 20 guide RNAs for editing enzymatic kinetic using ADAR2 is 12.

FIG. 40 shows Venn diagrams summarizing the number of guide RNAs that provided on-target nucleotide editing at the target nucleotide of a target RNA SERPINA1 when using ADAR2 (a, >70% on target editing at the 100 min time point; b, <70% off-target editing at the 100 min time point; c, sequencing read depth: >50); the number of guide RNAs that provided on-target at the target nucleotide of SERPINA1 when using ADAR1 (a, >40% on target editing at the 100 min time point; b, <40% off-target editing at the 100 min time point; c, sequencing read depth: >50); or the top 20 guide RNAs for editing enzymatic kinetic when using ADAR2 (a, >80% on target editing at the 100 min time point; b, enzymatic kinetic curve fit r²>0.8). 3 guide RNAs are provided on-target editing when using ADAR2. 10 guide RNAs are provided on-target when using ADAR1.0 guide RNA provided on-target editing when using ADAR1 and ADAR2.

FIG. 41A shows a summary of the kinetic rates of target nucleotide editing in a high throughput guide screening assay performed on a target RNA SERPINA1 and ADAR2 using 69000 guide RNA sequences. The X-axis shows the position of a base on the target RNA relative to the edit site. Position 0 is the target nucleotide. The number on the right of the target nucleotide indicates the nucleotides downstream of the target nucleotide. The number on the left of the target nucleotide indicates the nucleotides upstream of the target nucleotide. The Y-axis lists the guide RNAs tested. The color indicates the kinetics of the editing; a lighter color indicates faster kinetics while a darker color indicates slower kinetics.

FIG. 41B shows the frequency of editing of an optimized guide RNA for a target RNA SERPINA1 using a guide RNA with A-C mismatch or an optimized high-ranked engineered design and ADAR2. The Y-axis shows the percent editing frequency of various positions of the target RNA. The X-axis shows various positions of the target RNA. The top plot shows that at 30 minutes time point, the guide RNA with the A-C mismatch provided high on-target and high off-target editing. The bottom plot shows that the guide RNA with the optimized high-ranked engineered design (from library 2 with about 69,000 unbiased guide RNA designs) provided high on-target and low off-target editing.

FIG. 42 shows constructs of piggyBac vectors carrying a LRRK2 minigene having a G2019S mutation and mCherry (at top) or a carrying a LRRK2 minigene having a G2019S mutation, mCherry, CMV, and ADAR2 (at bottom).

FIG. 43A shows in vitro on and off-target editing of the LRRK2 G2019S mutation by ADAR1 after administration of two guide RNAs and a control (GFP plasmid).

FIG. 43B shows in vitro on and off-target editing of the LRRK2 G2019S mutation by ADAR1 and ADAR2 after administration of two guide RNAs and a control (GFP plasmid).

FIG. 44A shows percent RNA editing for constructs encoding a guide RNA targeting a mutation in ABCA4, an SmOPT sequence, and a U7 hairpin, where expression is driven by a U1 promoter. FIG. 44B shows Sanger sequencing traces for the various constructs shown in FIG. 44A.

FIG. 45A shows percent RNA editing in cells by ADAR1 and ADAR2 for multiple doses of constructs encoding a guide RNA targeting a mutation in ABCA4. FIG. 45B shows percent RNA editing in cells by ADAR1 for multiple doses of constructs encoding a guide RNA targeting a mutation in ABCA4.

FIG. 46A shows RNA editing of the ABCA4 G5882A missense mutation facilitated by engineered polynucleotides encoding U1 promoter driven guide RNAs in HEK293 cells. The target A to be edited is positioned at the center of the guide RNA (0.100.50) or is positioned 81 nucleotides in from the 5′ end of the guide RNA (0.100.80). FIG. 46B shows structures of various guides.

FIG. 47A and FIG. 47B show heatmaps illustrating percent RNA editing of the ABCA4 G5882A missense mutation facilitated by engineered polynucleotides encoding U1 promoter driven guide RNAs. RNA editing was tested in HEK293 cells naturally expressing ADAR1 and transfected with an ABCA4 minigene and transfected to overexpress ADAR2. Heatmaps show the target A to be edited and an off-target A immediately 3′ of the target A to be edited.

FIG. 48 shows placement a graph showing on-target and off-target editing of the ABCA4 G5882A missense mutation facilitated by engineered polynucleotides encoding U1 promoter driven guide RNAs with an SmOPT sequence and a U7 hairpin. Below the graph is a schematic showing the structure of the guide RNA with the observed pattern of on-target and off-target editing. Symmetric 4/4 internal loops were placed near off-target editing activity as a strategy to reduce off-target editing.

FIG. 49 shows structures of target RNA bound to various guide RNAs generated from the guide RNA in FIG. 48 modified with symmetric 5/5 internal loops or symmetric 4/4/internal loops placed near off-target editing activity. Delta G values for each guide RNA are shown at right. All guides were encoded for by a construct encoding SmOPT and U7 hairpin. Guides were under the control of a U1 promoter.

FIG. 50 shows ADAR1 editing in HEK293 cells of the ABCA4 G5882A missense mutation facilitated by the engineered guide RNAs of FIG. 49. All guides were encoded for by a construct encoding SmOPT and U7 hairpin. Guides were under the control of a U1 promoter.

FIG. 51 shows ADAR1 and ADAR2 editing in HEK293 cells of the ABCA4 G5882A missense mutation facilitated by the engineered guide RNAs of FIG. 49. All guides were encoded for by a construct encoding SmOPT and U7 hairpin. Guides were under the control of a U1 promoter.

FIG. 52 show a plot of RNA editing of guide Exb70 at the target A to be edited (“0” on the x-axis) and at RNA editing at off-target positions (represented as black bars at positions that are not “0”).

FIG. 53 show a plot of RNA editing of guide Exb71 at the target A to be edited (“0” on the x-axis) and at RNA editing at off-target positions (represented as black bars at positions that are not “0”).

FIG. 54 show a plot of RNA editing of guide Exb72 at the target A to be edited (“0” on the x-axis) and at RNA editing at off-target positions (represented as black bars at positions that are not “0”).

FIG. 55 show a plot of RNA editing of guide Exb73 at the target A to be edited (“0” on the x-axis) and at RNA editing at off-target positions (represented as black bars at positions that are not “0”).

FIG. 56 show a plot of RNA editing of guide Exb74 at the target A to be edited (“0” on the x-axis) and at RNA editing at off-target positions (represented as black bars at positions that are not “0”).

FIG. 57 show a plot of RNA editing of guide Exb93 at the target A to be edited (“0” on the x-axis) and at RNA editing at off-target positions (represented as black bars at positions that are not “0”).

FIG. 58 show a plot of RNA editing of guide Exb94 at the target A to be edited (“0” on the x-axis) and at RNA editing at off-target positions (represented as black bars at positions that are not “0”).

FIG. 59 show a plot of RNA editing of guide Exb95 at the target A to be edited (“0” on the x-axis) and at RNA editing at off-target positions (represented as black bars at positions that are not “0”).

FIG. 60 show a plot of RNA editing of guide Exb96 at the target A to be edited (“0” on the x-axis) and at RNA editing at off-target positions (represented as black bars at positions that are not “0”).

FIG. 61 show a plot of RNA editing of guide Exb98 at the target A to be edited (“0” on the x-axis) and at RNA editing at off-target positions (represented as black bars at positions that are not “0”).

FIG. 62 show a plot of RNA editing of guide Exb99 at the target A to be edited (“0” on the x-axis) and at RNA editing at off-target positions (represented as black bars at positions that are not “0”).

FIG. 63 show a plot of RNA editing of guide Exb100 at the target A to be edited (“0” on the x-axis) and at RNA editing at off-target positions (represented as black bars at positions that are not “0”).

FIG. 64 show a plot of RNA editing of guide Exb101 at the target A to be edited (“0” on the x-axis) and at RNA editing at off-target positions (represented as black bars at positions that are not “0”).

FIG. 65 show a plot of RNA editing of Guide 1-151 at the target A to be edited (“0” on the x-axis) and at RNA editing at off-target positions (represented as black bars at positions that are not “0”).

FIG. 66 show a plot of RNA editing of Guide 2 at the target A to be edited (“0” on the x-axis) and at RNA editing at off-target positions (represented as black bars at positions that are not “0”).

FIG. 67 show a plot of RNA editing of Guide 3 at the target A to be edited (“0” on the x-axis) and at RNA editing at off-target positions (represented as black bars at positions that are not “0”).

FIG. 68 show a plot of RNA editing of Guide 4 at the target A to be edited (“0” on the x-axis) and at RNA editing at off-target positions (represented as black bars at positions that are not “0”).

FIG. 69 show a plot of RNA editing of Guide 5 at the target A to be edited (“0” on the x-axis) and at RNA editing at off-target positions (represented as black bars at positions that are not “0”).

FIG. 70 show a plot of RNA editing of Guide 6 at the target A to be edited (“0” on the x-axis) and at RNA editing at off-target positions (represented as black bars at positions that are not “0”).

FIG. 71 show a plot of RNA editing of Guide 7 at the target A to be edited (“0” on the x-axis) and at RNA editing at off-target positions (represented as black bars at positions that are not “0”).

FIG. 72 show a plot of RNA editing of Guide 8 at the target A to be edited (“0” on the x-axis) and at RNA editing at off-target positions (represented as black bars at positions that are not “0”).

FIG. 73 show a plot of RNA editing of Guide 9 at the target A to be edited (“0” on the x-axis) and at RNA editing at off-target positions (represented as black bars at positions that are not “0”).

FIG. 74 show a plot of RNA editing of Guide 10 at the target A to be edited (“0” on the x-axis) and at RNA editing at off-target positions (represented as black bars at positions that are not “0”).

FIG. 75 show a plot of RNA editing of Guide 11 at the target A to be edited (“0” on the x-axis) and at RNA editing at off-target positions (represented as black bars at positions that are not “0”).

FIG. 76 show a plot of RNA editing of Guide 12 at the target A to be edited (“0” on the x-axis) and at RNA editing at off-target positions (represented as black bars at positions that are not “0”).

FIG. 77 show a plot of RNA editing of Guide 14 at the target A to be edited (“0” on the x-axis) and at RNA editing at off-target positions (represented as black bars at positions that are not “0”).

FIG. 78 show a plot of RNA editing of Guide 15 (gRNA 8) at the target A to be edited (“0” on the x-axis) and at RNA editing at off-target positions (represented as black bars at positions that are not “0”).

FIG. 79 show a plot of RNA editing of Guide 16 at the target A to be edited (“0” on the x-axis) and at RNA editing at off-target positions (represented as black bars at positions that are not “0”).

FIG. 80 show a plot of RNA editing of Guide 18 (exb100 mirror) at the target A to be edited (“0” on the x-axis) and at RNA editing at off-target positions (represented as black bars at positions that are not “0”).

FIG. 81 show a plot of RNA editing of Guide 19 at the target A to be edited (“0” on the x-axis) and at RNA editing at off-target positions (represented as black bars at positions that are not “0”).

FIG. 82 show a plot of RNA editing of Guide 20 at the target A to be edited (“0” on the x-axis) and at RNA editing at off-target positions (represented as black bars at positions that are not “0”).

FIG. 83 show a plot of RNA editing of Guide 21 (exb101 mirror) at the target A to be edited (“0” on the x-axis) and at RNA editing at off-target positions (represented as black bars at positions that are not “0”).

FIG. 84 show a plot of RNA editing of Guide 22 at the target A to be edited (“0” on the x-axis) and at RNA editing at off-target positions (represented as black bars at positions that are not “0”).

FIG. 85 show a plot of RNA editing of Guide 23 at the target A to be edited (“0” on the x-axis) and at RNA editing at off-target positions (represented as black bars at positions that are not “0”).

FIG. 86 show a plot of RNA editing of Guide 24 at the target A to be edited (“0” on the x-axis) and at RNA editing at off-target positions (represented as black bars at positions that are not “0”).

FIG. 87 show a plot of RNA editing of Guide 25 at the target A to be edited (“0” on the x-axis) and at RNA editing at off-target positions (represented as black bars at positions that are not “0”).

FIG. 88 show a plot of RNA editing of Guide 26 at the target A to be edited (“0” on the x-axis) and at RNA editing at off-target positions (represented as black bars at positions that are not “0”).

FIG. 89 show a plot of RNA editing of Guide 27 at the target A to be edited (“0” on the x-axis) and at RNA editing at off-target positions (represented as black bars at positions that are not “0”).

FIG. 90 show a plot of RNA editing of Guide 28 at the target A to be edited (“0” on the x-axis) and at RNA editing at off-target positions (represented as black bars at positions that are not “0”).

FIG. 91 show a plot of RNA editing of Guide 29 at the target A to be edited (“0” on the x-axis) and at RNA editing at off-target positions (represented as black bars at positions that are not “0”).

FIG. 92 show a plot of RNA editing of Guide 30 at the target A to be edited (“0” on the x-axis) and at RNA editing at off-target positions (represented as black bars at positions that are not “0”).

FIG. 93 show a plot of RNA editing of Guide 31 at the target A to be edited (“0” on the x-axis) and at RNA editing at off-target positions (represented as black bars at positions that are not “0”).

FIG. 94 show a plot of RNA editing of Guide 32 at the target A to be edited (“0” on the x-axis) and at RNA editing at off-target positions (represented as black bars at positions that are not “0”).

FIG. 95 shows a comparison between RNA editing efficiencies for guides Exb95 and Exb94.

FIG. 96 shows replicate experiments assessing percent RNA editing achieved by a guide RNA that forms structural features upon hybridization to ABCA4

FIG. 97 shows editing of SERPINA1 minigenes 1 and 2 using guide RNAs expressed using a U6 or U7 promoter with a 3′ SmOPT hU7 hairpin.

FIG. 98 shows a plot of RNA editing of SERPINA1 for the guide RNAs listed as SEQ ID NO: 102 and SEQ ID NO: 103 at the target A to be edited (“0” on the x-axis) and at RNA editing at off-target positions (represented as black bars at positions that are not “0”)

FIG. 99 shows plots of off target editing profiles of an Exb75 circular guide for the target SNCA and a depiction of the guide.

FIG. 100 shows plots of off target editing profiles of an Exb76 circular guide for the target SNCA and a depiction of the guide.

FIG. 101 shows plots of off target editing profiles of an Exb77 circular guide for the target SNCA and a depiction of the guide.

FIG. 102 shows plots of off target editing profiles of an Exb78 circular guide for the target SNCA and a depiction of the guide.

FIG. 103 shows plots of off target editing profiles of an Exb79 circular guide for the target SNCA and a depiction of the guide.

FIG. 104 shows an exemplary control guide02_TTHY2_v0093 RNA design for targeting LRRK2 and the editing at the target A to be edited (“0” on the x-axis) and at RNA editing at off-target positions (represented as black bars at positions that are not “0”) at 100 min.

FIG. 105 shows the percentage editing as a function of time as determined by sequencing for exemplary control guide02_TTHY2_v0093.

FIG. 106 shows editing at the target A to be edited (“0” on the x-axis) and at RNA editing at off-target positions (represented as black bars at positions that are not “0”) at 1 min, 10 min, 30 min, and 100 min for exemplary control guide02_TTHY2_v0093.

FIG. 107 shows an exemplary control guide03_Glu2bRG_v0090 RNA design for targeting LRRK2 and the editing at the target A to be edited (“0” on the x-axis) and at RNA editing at off-target positions (represented as black bars at positions that are not “0”) at 100 min.

FIG. 108 shows the percentage editing as a function of time as determined by sequencing for exemplary control guide03_Glu2bRG_v0090.

FIG. 109 shows editing at the target A to be edited (“0” on the x-axis) and at RNA editing at off-target positions (represented as black bars at positions that are not “0”) at 1 min, 10 min, 30 min, and 100 min for exemplary control guide03_Glu2bRG_v0090.

FIG. 110 shows an exemplary guide10_Glu2bQR_v0446 RNA design for targeting LRRK2 and the editing at the target A to be edited (“0” on the x-axis) and at RNA editing at off-target positions (represented as black bars at positions that are not “0”) at 100 min.

FIG. 111 shows the percentage editing as a function of time as determined by sequencing for exemplary guide10_Glu2bQR_v0446.

FIG. 112 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0446.

FIG. 113 shows an exemplary guide 11_Glu2bQR_v0262 RNA design for targeting LRRK2 and the editing at the target A to be edited (“0” on the x-axis) and at RNA editing at off-target positions (represented as black bars at positions that are not “0”) at 100 min.

FIG. 114 shows the percentage editing as a function of time as determined by sequencing for exemplary guide11_Glu2bQR_v0262.

FIG. 116 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0262.

FIG. 116 shows an exemplary guide10_Glu2bQR_v0022 RNA design for targeting LRRK2 and the editing at the target A to be edited (“0” on the x-axis) and at RNA editing at off-target positions (represented as black bars at positions that are not “0”) at 100 min.

FIG. 117 shows the percentage editing as a function of time as determined by sequencing for exemplary guide10_Glu2bQR_v0022.

FIG. 118 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0022.

FIG. 119 shows an exemplary guide4_Glu2bRG_v0094 RNA design for targeting LRRK2 and the editing at the target A to be edited (“0” on the x-axis) and at RNA editing at off-target positions (represented as black bars at positions that are not “0”) at 100 min.

FIG. 120 shows the percentage editing as a function of time as determined by sequencing for exemplary guide4_Glu2bRG_v0094.

FIG. 121 shows editing at the target A to be edited (“0” on the x-axis) and at RNA editing at off-target positions (represented as black bars at positions that are not “0”) at 1 min, 10 min, 30 min, and 100 min for exemplary guide4_Glu2bRG_v0094.

FIG. 122 shows an exemplary guide4_Glu2bRG_v0126 RNA design for targeting LRRK2 and the editing at the target A to be edited (“0” on the x-axis) and at RNA editing at off-target positions (represented as black bars at positions that are not “0”) at 100 min.

FIG. 123 shows the percentage editing as a function of time as determined by sequencing for exemplary guide4_Glu2bRG_v0126.

FIG. 124 shows editing at the target A to be edited (“0” on the x-axis) and at RNA editing at off-target positions (represented as black bars at positions that are not “0”) at 1 min, 10 min, 30 min, and 100 min for exemplary guide4_Glu2bRG_v0126.

FIG. 125 shows an exemplary guide11_Glu2bQR_v0278 RNA design for targeting LRRK2 and the editing at the target A to be edited (“0” on the x-axis) and at RNA editing at off-target positions (represented as black bars at positions that are not “0”) at 100 min.

FIG. 126 shows the percentage editing as a function of time as determined by sequencing for exemplary guide11_Glu2bQR_v0278.

FIG. 127 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0278.

FIG. 128 shows an exemplary guide10_Glu2bQR_v0270 RNA design for targeting LRRK2 and the editing at the target A to be edited (“0” on the x-axis) and at RNA editing at off-target positions (represented as black bars at positions that are not “0”) at 100 min.

FIG. 129 shows the percentage editing as a function of time as determined by sequencing for exemplary guide10_Glu2bQR_v0270.

FIG. 130 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0270.

FIG. 131 shows an exemplary guide10_Glu2bQR_v0398 RNA design for targeting LRRK2 and the editing at the target A to be edited (“0” on the x-axis) and at RNA editing at off-target positions (represented as black bars at positions that are not “0”) at 100 min.

FIG. 132 shows the percentage editing as a function of time as determined by sequencing for exemplary guide10_Glu2bQR_v0398.

FIG. 133 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0398.

FIG. 134 shows an exemplary guide10_Glu2bQR_v0314 RNA design for targeting LRRK2 and the editing at the target A to be edited (“0” on the x-axis) and at RNA editing at off-target positions (represented as black bars at positions that are not “0”) at 100 min.

FIG. 135 shows the percentage editing as a function of time as determined by sequencing for exemplary guide10_Glu2bQR_v0314.

FIG. 136 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0314.

FIG. 137 shows an exemplary guide10_Glu2bQR_v0142 RNA design for targeting LRRK2 and the editing at the target A to be edited (“0” on the x-axis) and at RNA editing at off-target positions (represented as black bars at positions that are not “0”) at 100 min.

FIG. 138 shows the percentage editing as a function of time as determined by sequencing for exemplary guide10_Glu2bQR_v0142.

FIG. 139 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0142.

FIG. 140 shows an exemplary guide10_Glu2bQR_v0510 RNA design for targeting LRRK2 and the editing at the target A to be edited (“0” on the x-axis) and at RNA editing at off-target positions (represented as black bars at positions that are not “0”) at 100 min.

FIG. 141 shows the percentage editing as a function of time as determined by sequencing for exemplary guide10_Glu2bQR_v0510.

FIG. 142 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0510.

FIG. 143 shows an exemplary guide11_Glu2bQR_v0310 RNA design for targeting LRRK2 and the editing at the target A to be edited (“0” on the x-axis) and at RNA editing at off-target positions (represented as black bars at positions that are not “0”) at 100 min.

FIG. 144 shows the percentage editing as a function of time as determined by sequencing for exemplary guide11_Glu2bQR_v0310.

FIG. 145 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0310.

FIG. 146 shows an exemplary guide10_Glu2bQR_v0262 RNA design for targeting LRRK2 and the editing at the target A to be edited (“0” on the x-axis) and at RNA editing at off-target positions (represented as black bars at positions that are not “0”) at 100 min.

FIG. 147 shows the percentage editing as a function of time as determined by sequencing for exemplary guide10_Glu2bQR_v0262.

FIG. 148 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0262.

FIG. 149 shows an exemplary guide10_Glu2bQR_v0134 RNA design for targeting LRRK2 and the editing at the target A to be edited (“0” on the x-axis) and at RNA editing at off-target positions (represented as black bars at positions that are not “0”) at 100 min.

FIG. 150 shows the percentage editing as a function of time as determined by sequencing for exemplary guide10_Glu2bQR_v0134.

FIG. 151 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0134.

FIG. 152 shows an exemplary guide11_Glu2bQR_v0070 RNA design for targeting LRRK2 and the editing at the target A to be edited (“0” on the x-axis) and at RNA editing at off-target positions (represented as black bars at positions that are not “0”) at 100 min.

FIG. 153 shows the percentage editing as a function of time as determined by sequencing for exemplary guide11_Glu2bQR_v0070.

FIG. 154 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0070.

FIG. 155 shows an exemplary guide11_Glu2bQR_v0038 RNA design for targeting LRRK2 and the editing at the target A to be edited (“0” on the x-axis) and at RNA editing at off-target positions (represented as black bars at positions that are not “0”) at 100 min.

FIG. 156 shows the percentage editing as a function of time as determined by sequencing for exemplary guide11_Glu2bQR_v0038.

FIG. 157 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0038.

FIG. 158 shows an exemplary guide10_Glu2bQR_v0298 RNA design for targeting LRRK2 and the editing at the target A to be edited (“0” on the x-axis) and at RNA editing at off-target positions (represented as black bars at positions that are not “0”) at 100 min.

FIG. 159 shows the percentage editing as a function of time as determined by sequencing for exemplary guide10_Glu2bQR_v0298.

FIG. 160 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0298.

FIG. 161 shows an exemplary guide10_Glu2bQR_v0294 RNA design for targeting LRRK2 and the editing at the target A to be edited (“0” on the x-axis) and at RNA editing at off-target positions (represented as black bars at positions that are not “0”) at 100 min.

FIG. 162 shows the percentage editing as a function of time as determined by sequencing for exemplary guide10_Glu2bQR_v0294.

FIG. 163 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0294.

FIG. 164 shows an exemplary guide10_Glu2bQR_v0038 RNA design for targeting LRRK2 and the editing at the target A to be edited (“0” on the x-axis) and at RNA editing at off-target positions (represented as black bars at positions that are not “0”) at 100 min.

FIG. 165 shows the percentage editing as a function of time as determined by sequencing for exemplary guide10_Glu2bQR_v0038.

FIG. 166 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0038.

FIG. 167 shows an exemplary guide04_Glu2bRG_v0118 RNA design for targeting LRRK2 and the editing at the target A to be edited (“0” on the x-axis) and at RNA editing at off-target positions (represented as black bars at positions that are not “0”) at 100 min.

FIG. 168 shows the percentage editing as a function of time as determined by sequencing for exemplary guide04_Glu2bRG_v0118.

FIG. 169 shows editing at the target A to be edited (“0” on the x-axis) and at RNA editing at off-target positions (represented as black bars at positions that are not “0”) at 1 min, 10 min, 30 min, and 100 min for exemplary guide04_Glu2bRG_v0118.

FIG. 170 shows an exemplary guide11_Glu2bQR_v0326 RNA design for targeting LRRK2 and the editing at the target A to be edited (“0” on the x-axis) and at RNA editing at off-target positions (represented as black bars at positions that are not “0”) at 100 min.

FIG. 171 shows the percentage editing as a function of time as determined by sequencing for exemplary guide11_Glu2bQR_v0326.

FIG. 172 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0326.

FIG. 173 shows an exemplary guide11_Glu2bQR_v0054 RNA design for targeting LRRK2 and the editing at the target A to be edited (“0” on the x-axis) and at RNA editing at off-target positions (represented as black bars at positions that are not “0”) at 100 min.

FIG. 174 shows the percentage editing as a function of time as determined by sequencing for exemplary guide11_Glu2bQR_v0054.

FIG. 175 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0054.

FIG. 176 shows an exemplary guide11_Glu2bQR_v0390 RNA design for targeting LRRK2 and the editing at the target A to be edited (“0” on the x-axis) and at RNA editing at off-target positions (represented as black bars at positions that are not “0”) at 100 min.

FIG. 177 shows the percentage editing as a function of time as determined by sequencing for exemplary guide11_Glu2bQR_v0390.

FIG. 178 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0390.

FIG. 179 shows an exemplary guide03_Glu2bRG_v0014 RNA design for targeting LRRK2 and the editing at the target A to be edited (“0” on the x-axis) and at RNA editing at off-target positions (represented as black bars at positions that are not “0”) at 100 min.

FIG. 180 shows the percentage editing as a function of time as determined by sequencing for exemplary guide03_Glu2bRG_v0014.

FIG. 181 shows editing at the target A to be edited (“0” on the x-axis) and at RNA editing at off-target positions (represented as black bars at positions that are not “0”) at 1 min, 10 min, 30 min, and 100 min for exemplary guide03_Glu2bRG_v0014.

FIG. 182 shows an exemplary guide10_Glu2bQR_v0430 RNA design for targeting LRRK2 and the editing at the target A to be edited (“0” on the x-axis) and at RNA editing at off-target positions (represented as black bars at positions that are not “0”) at 100 min.

FIG. 183 shows the percentage editing as a function of time as determined by sequencing for exemplary guide10_Glu2bQR_v0430.

FIG. 184 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0430.

FIG. 185 shows an exemplary guide10_Glu2bQR_v0318 RNA design for targeting LRRK2 and the editing at the target A to be edited (“0” on the x-axis) and at RNA editing at off-target positions (represented as black bars at positions that are not “0”) at 100 min.

FIG. 186 shows the percentage editing as a function of time as determined by sequencing for exemplary guide10_Glu2bQR_v0318.

FIG. 187 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0318.

FIG. 188 shows an exemplary guide10_Glu2bQR_v0006 RNA design for targeting LRRK2 and the editing at the target A to be edited (“0” on the x-axis) and at RNA editing at off-target positions (represented as black bars at positions that are not “0”) at 100 min.

FIG. 189 shows the percentage editing as a function of time as determined by sequencing for exemplary guide10_Glu2bQR_v0006.

FIG. 190 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0006.

FIG. 191 shows an exemplary guide11_Glu2bQR_v0022 RNA design for targeting LRRK2 and the editing at the target A to be edited (“0” on the x-axis) and at RNA editing at off-target positions (represented as black bars at positions that are not “0”) at 100 min.

FIG. 192 shows the percentage editing as a function of time as determined by sequencing for exemplary guide11_Glu2bQR_v0022.

FIG. 193 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0022.

FIG. 194 shows an exemplary guide10_Glu2bQR_v0414 RNA design for targeting LRRK2 and the editing at the target A to be edited (“0” on the x-axis) and at RNA editing at off-target positions (represented as black bars at positions that are not “0”) at 100 min.

FIG. 195 shows the percentage editing as a function of time as determined by sequencing for exemplary guide10_Glu2bQR_v0414.

FIG. 196 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0414.

FIG. 197 shows an exemplary guide10_Glu2bQR_v0302 RNA design for targeting LRRK2 and the editing at the target A to be edited (“0” on the x-axis) and at RNA editing at off-target positions (represented as black bars at positions that are not “0”) at 100 min.

FIG. 198 shows the percentage editing as a function of time as determined by sequencing for exemplary guide10_Glu2bQR_v0302.

FIG. 199 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0302.

FIG. 200 shows an exemplary guide10_Glu2bQR_v0494 RNA design for targeting LRRK2 and the editing at the target A to be edited (“0” on the x-axis) and at RNA editing at off-target positions (represented as black bars at positions that are not “0”) at 100 min.

FIG. 201 shows the percentage editing as a function of time as determined by sequencing for exemplary guide10_Glu2bQR_v0494.

FIG. 202 shows editing at the target A to be edited (“0” on the x-axis) and at RNA editing at off-target positions (represented as black bars at positions that are not “0”) at 1 min, 10 min, 30 min, and 100 min for exemplary guide10_Glu2bQR_v0494.

FIG. 203 shows an exemplary guide11_Glu2bQR_v0134 RNA design for targeting LRRK2 and the editing at the target A to be edited (“0” on the x-axis) and at RNA editing at off-target positions (represented as black bars at positions that are not “0”) at 100 min.

FIG. 204 shows the percentage editing as a function of time as determined by sequencing for exemplary guide11_Glu2bQR_v0134.

FIG. 205 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0134.

FIG. 206 shows an exemplary guide11_Glu2bQR_v0006 RNA design for targeting LRRK2 and the editing at the target A to be edited (“0” on the x-axis) and at RNA editing at off-target positions (represented as black bars at positions that are not “0”) at 100 min.

FIG. 207 shows the percentage editing as a function of time as determined by sequencing for exemplary guide11_Glu2bQR_v0006.

FIG. 208 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0006.

FIG. 209 shows an exemplary guide11_Glu2bQR_v0294 RNA design for targeting LRRK2 and the editing at the target A to be edited (“0” on the x-axis) and at RNA editing at off-target positions (represented as black bars at positions that are not “0”) at 100 min.

FIG. 210 shows the percentage editing as a function of time as determined by sequencing for exemplary guide11_Glu2bQR_v0294.

FIG. 211 shows editing at the target A to be edited (“0” on the x-axis) and at RNA editing at off-target positions (represented as black bars at positions that are not “0”) at 1 min, 10 min, 30 min, and 100 min for exemplary guide11_Glu2bQR_v0294.

FIG. 212 shows heat maps and structures for exemplary engineered guide RNA sequences targeting a LRRK2 mRNA. The heat map provides visualization of the editing profile at the 10 minute time point. 5 engineered guide RNAs for on-target editing (with no-2 filter) are in the left graph and 5 engineered guide RNAs for on-target editing with minimal-2 editing are depicted on the right graph. The corresponding predicted secondary structures are below the heat maps.

FIG. 213 shows exemplary engineered guide RNAs comprising a dumbbell design and that target LRRK2 mRNA.

FIG. 214 shows graphs of on-target and off-target ADAR1 (left side) and ADAR1+ADAR2 (right side) editing of LRRK2 for the 871 113.57 (top) and 860 113.57 (bottom) guides of FIG. 213.

FIG. 215 shows graphs of on-target and off-target ADAR1 (left side) and ADAR1+ADAR2 (right side) editing of LRRK2 for the 1976 113.57 (top) and 919 113.57 (bottom) guides of FIG. 213.

FIG. 216 shows graphs of on-target and off-target ADAR1 (left side) and ADAR1+ADAR2 (right side) editing of LRRK2 for the 2108 113.57 (top) and 1700 113.57 (bottom) guides of FIG. 213.

FIG. 217 shows graphs of on-target and off-target ADAR1 (left side) and ADAR1+ADAR2 (right side) editing of LRRK2 for the 844 113.57 guide of FIG. 213.

FIG. 218 shows the sequence and structure of the following ABCA4 guides bound to target: guide01_CAPS1_128_gID_00001_v0114; guide06_Shaker5G_256_gID_01981_v0156; guide06_Shaker5G_256_gID_01981_v0025; guide06_Shaker5G_256_gID_01981_v0220; guide01_CAPS1_128_gID_00001_v0115; guide01_CAPS1_128_gID_00001_v0081; guide01_CAPS1_128_gID_00001_v0019; and guide06_Shaker5G_256_gID_01981_v0153.

FIG. 219 shows the sequence and structure of the following ABCA4 guides bound to target: guide05_Shaker5G_256_gID_01585_v0027; guide08_AJUBA_512_gID_02773_v0446; guide06_Shaker5G_256_gID_01981_v0025; guide08_AJUBA_512_gID_02773_v0414; guide01_CAPS1_128_gID_00001_v0018; guide06_Shaker5G_256_gID_01981_v0154; guide01_CAPS1_128_gID_00001_v0052; and guide01_CAPS1_128_gID_00001_v0050.

FIG. 220 shows the sequence and structure of the following ABCA4 guides bound to target: guide08_AJUBA_512_gID_02773_v0190; guide08_AJUBA_512_gID_02773_v0445; guide01_CAPS1_128_gID_00001_v0116; guide06_Shaker5G_256_gID_01981_v0028; guide08_AJUBA_512_gID_02773_v0062; guide08_AJUBA_512_gID_02773_v0189; guide01_CAPS1_128_gID_00001_v0082; and guide08_AJUBA_512_gID_02773_v0142.

FIG. 221 shows the sequence and structure of the following ABCA4 guides bound to target: guide05_Shaker5G_256_gID_01585_v0155; guide06_Shaker5G_256_gID_01981_v0155; guide01_CAPS1_128_gID_00001_v0113; guide01_CAPS1_128_gID_00001 v0030; guide01_CAPS1_128_gID_00001_v0084; guide01_CAPS1_128_gID_00001 v0049; guide01_CAPS1_128_gID_00001_v0020; and guide01_CAPS1_128_gID_00001_v0051.

FIG. 222 shows editing at the target A to be edited (“0” on the x-axis) and at RNA editing at off-target positions (represented as black bars at positions that are not “0”) at 100 min with ADAR1 (top), 100 min with ADAR2 (second to top); and at 1 min, 10 min, 30 min, and 100 min with ADAR2 (descending) for exemplary guide01_CAPS1_128_gID_00001_v0073.

FIG. 223 shows editing at the target A to be edited (“0” on the x-axis) and at RNA editing at off-target positions (represented as black bars at positions that are not “0”) at 100 min with ADAR1 (top), 100 min with ADAR2 (second to top); and at 1 min, 10 min, 30 min, and 100 min with ADAR2 (descending) for exemplary guide08_AJUBA_512_gID_02773_v0268.

FIG. 224 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0114.

FIG. 225 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide06_Shaker5G_256_gID_01981_v0156.

FIG. 226 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide06_Shaker5G_256_gID_01981_v0025.

FIG. 227 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide06_Shaker5G_256_gID_01981_v0220.

FIG. 228 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0115.

FIG. 229 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0081.

FIG. 230 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0019.

FIG. 231 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide06_Shaker5G_256_gID_01981_v0153.

FIG. 232 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide05_Shaker5G_256_gID_01585_v0027.

FIG. 233 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide08_AJUBA_512_gID_02773_v0446.

FIG. 234 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide06_Shaker5G_256_gID_01981_v0026.

FIG. 235 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide08_AJUBA_512_gID_02773_v0414.

FIG. 236 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0018.

FIG. 237 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide06_Shaker5G_256_gID_01981_v0154.

FIG. 238 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0052.

FIG. 239 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0050.

FIG. 240 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide08_AJUBA_512_gID_02773_v0190.

FIG. 241 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide08_AJUBA_512_gID_02773_v0445.

FIG. 242 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0116.

FIG. 243 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide06_Shaker5G_256_gID_01981_v0028.

FIG. 244 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide08_AJUBA_512_gID_02773_v0062.

FIG. 245 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide08_AJUBA_512_gID_02773_v0189.

FIG. 246 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0082.

FIG. 247 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide08_AJUBA_512_gID_02773_v0142.

FIG. 248 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide05_Shaker5G_256_gID_01585_v0155.

FIG. 249 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide06_Shaker5G_256_gID_01981_v0155.

FIG. 250 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0113.

FIG. 251 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0030.

FIG. 252 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0084.

FIG. 253 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0049.

FIG. 254 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0020.

FIG. 255 shows editing at the target A to be edited (“0” on the x-axis) and at RNA editing at off-target positions (represented as black bars at positions that are not “0”) at 100 min with ADAR1 (top left), 100 min with ADAR2 (second to top left); and at 1 min, 10 min, 30 min, and 100 min with ADAR2 (descending); editing with ADAR1 as a function of time (top right); and editing with ADAR2 as a function of time (second to top right) for exemplary guide01_CAPS1_128_gID_00001_v0051.

FIG. 256 shows a depiction of a first engineered guide RNA (top) that forms a single mismatch with the target SERPINA1 mRNA sequence and a second exemplary engineered guide RNA (bottom) targeting SERPINA1 mRNA, where the second engineered guide RNA forms two mismatches with the target SERPINA1 mRNA sequence.

FIG. 257 (left) shows that the constructs containing the U7 promoter, U7 hairpin, and the SmOPT sequences exhibited the highest levels of on-target RNA editing. FIG. 257 (right) shows that the second engineered guide RNA, which formed an A/C and A/A mismatch upon hybridization to SERPINA1 mRNA, exhibited less local off-target editing.

FIG. 258 (left) depicts editing of SERPINA1 mRNA with various guides as a function of guide length at 24 hours and 48 hours. FIG. 258 (right) shows the editing at the target A to be edited (“0” on the x-axis) and at RNA editing at off-target positions (represented as black bars at positions that are not “0”) at 24 hours and 48 hours with 95 nucleotide and 100 nucleotide SERPINA1 guides.

FIG. 259 (left) depicts editing of SERPINA1 mRNA with three exemplary guides. FIG. 259 (right) shows the editing at the target A to be edited (“0” on the x-axis) and at RNA editing at off-target positions (represented as black bars at positions that are not “0”) with the three exemplary SERPINA1 guides.

FIG. 260 (left) depicts editing of SERPINA1 mRNA with various guides as a function of guide length spanning 95, 99, 103, 107, 111, 115, 119, and 123 nucleotides. FIG. 260 (top right) depicts editing of SERPINA1 mRNA with various guides of guide length of 107, 111, and 119 nucleotides, with and without introduction of a bulge. FIG. 260 (bottom right) shows the editing at the target A to be edited (“0” on the x-axis) and at RNA editing at off-target positions (represented as black bars at positions that are not “0”) for the SERPINA1 guide.

FIG. 261 (right) shows a schematic of the SERPINA1 target sequence and oligo tether engineered guide RNAs. FIG. 261 (left) depicts editing of SERPINA1 mRNA with the engineered guide RNAs having oligo tethers.

FIG. 262 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side).

FIG. 263 shows a schematic of the structural features formed in the guide-target RNA scaffold of various engineered guide RNAs of the present disclosure targeting LRRK2.

FIG. 264 shows a schematic of the structural features formed in the guide-target RNA scaffold of various engineered guide RNAs of the present disclosure targeting LRRK2.

FIG. 265 shows a schematic of the structural features formed in the guide-target RNA scaffold of various engineered guide RNAs of the present disclosure targeting LRRK2.

### DETAILED DESCRIPTION

### RNA Editing Overview

RNA editing refers to a process by which RNA can be enzymatically modified post synthesis on specific nucleosides. RNA editing can comprise any one of an insertion, deletion, or substitution of a nucleotide(s). Examples of RNA editing include pseudouridylation (the isomerization of uridine residues) and deamination (removal of an amine group from cytidine to give rise to uridine or C-to-U editing through recruitment of an APOBEC enzyme described herein, or from adenosine to inosine or A-to-I editing through recruitment of an adenosine deaminase such as ADAR). Editing of RNA can be a way to modulate expression of a polypeptide, for example, through modulation of polypeptide-encoding double stranded RNA (“dsRNA” herein) substrates that enter the RNA interference (RNAi) pathway. This modulation can then act at the chromatin level to modulate expression of the polypeptide. Editing of RNA can also be a way to regulate gene translation. RNA editing can be a mechanism in which to regulate transcript recoding by regulating the triplet codon to introduce silent mutations and/or non-synonymous mutations.

Provided herein, in certain embodiments, are compositions that comprise engineered guide RNAs and engineered polynucleotides encoding the same that facilitate RNA editing via an RNA editing entity or a biologically active fragment thereof and methods of using the same. In an aspect, an RNA editing entity can comprise an adenosine Deaminase Acting on RNA (ADAR) and biologically active fragments thereof. In some instances, ADARs are enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it can be currently inconclusive if dimerization is needed for editing to occur.

Three human ADAR genes have been identified (ADARs 1-3) with ADAR1 (official symbol ADAR) and ADAR2 (ADARB1) proteins having well-characterized adenosine deamination activity. ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain.

Specific RNA editing can lead to transcript recoding. Because inosine shares the base pairing properties of guanosine, the translational machinery interprets edited adenosines as guanosine, altering the triplet codon, which can result in amino acid substitutions in protein products. More than half the triplet codons in the genetic code could be reassigned through RNA editing. Due to the degeneracy of the genetic code, RNA editing can cause both silent and non-synonymous amino acid substitutions.

In some cases, targeting an RNA can affect splicing. Adenosines targeted for editing can be disproportionately localized near splice junctions in pre-mRNA. Therefore, during formation of a dsRNA ADAR substrate, intronic cis-acting sequences can form RNA duplexes encompassing splicing sites and potentially obscuring them from the splicing machinery. Furthermore, through modification of select adenosines, ADARs can create or eliminate splicing sites, broadly affecting later splicing of the transcript. Similar to the translational machinery, the spliceosome interprets inosine as guanosine, and therefore, a canonical GU 5′ splice site and AG 3′ acceptor site can be created via the deamination of AU (IU=GU) and AA (AI=AG), respectively. Correspondingly, RNA editing can destroy a canonical AG 3′ splice site (IG=GG).

In some cases, targeting an RNA can affect microRNA (miRNA) production and function. For example, RNA editing of a pre-miRNA precursor can affect the abundance of a miRNA, RNA editing in the seed of the miRNA can redirect it to another target for translational repression, or RNA editing of a miRNA binding site in an RNA can interfere with miRNA complementarity, and thus interfere with suppression via RNAi.

In an aspect, an RNA editing entity can be recruited by a guide RNA of the present disclosure. In some examples, a guide RNA can recruit an RNA editing entity that, when associated with the guide RNA and a target RNA as described herein, facilitates: an editing of a base of a nucleotide of the target RNA, a modulation of the expression of a polypeptide encoded by a subject target RNA (such as LRRK2, SNCA, PINK1, Tau, and others described herein); or a combination thereof. A guide RNA can optionally contain an RNA editing entity recruiting domain capable of recruiting an RNA editing entity. In some embodiments, a guide RNA can lack an RNA editing entity recruiting domain and still be capable of binding an RNA editing entity, or be bound by it.

### RNA-Editing Systems

Disclosed herein are engineered guide RNAs and engineered polynucleotides encoding the same for site-specific, selective editing of a target RNA via an RNA editing entity or a biologically active fragment thereof. An engineered guide RNA of the present disclosure can comprise latent structures, such that when the engineered guide RNA is hybridized to the target RNA to form a guide-target RNA scaffold, at least a portion of the latent structure manifests as at least a portion of a structural feature as described herein.

An engineered guide RNA as described herein comprises a targeting domain with complementarity to a target RNA described herein. As such, a guide RNA can be engineered to site-specifically/selectively target and hybridize to a particular target RNA, thus facilitating editing of a specific target RNA via an RNA editing entity or a biologically active fragment thereof. The targeting domain can include a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch can help to localize editing of the RNA editing entity to the desired base of the target RNA. However, in some instances there can be some, and in some cases significant, off target editing in addition to the desired edit.

Hybridization of the target RNA and the targeting domain of the guide RNA produces specific secondary and tertiary structures in the guide-target RNA scaffold that manifest upon hybridization, which are referred to herein as “latent structures.” Latent structures when manifested become structural features described herein, including mismatches, bulges, internal loops, and hairpins. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination with the mismatch described above generally facilitate an increased amount of editing of a target adenosine, fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold can be a powerful tool to promote editing of a target RNA with high specificity, selectivity, and robust activity.

In some embodiments, the engineered guide RNAs of the present disclosure can be provided in an engineered RNA construct comprising other RNA elements, such as snRNA sequences, snRNA hairpins, or both. For example, an engineered RNA may comprise any engineered guide RNA disclosed herein and an SmOPT sequence, a U7 hairpin, or both. An SmOPT sequence, a U7 hairpin, or both may be positioned 5′ or 3′ of the engineered guide RNA.

### Engineered Guide RNAs

Provided herein are engineered guide RNAs with one or more latent structures (“latent structure guide RNAs”) that manifest as one or more structural features upon hybridization of the engineered guide RNA to a target RNA (for example, an RNA implicated in a disease or condition) and compositions comprising said engineered guide RNAs. As disclosed herein, the structural features in combination described herein generally facilitate an increased amount of editing of a target adenosine of the target RNA, fewer off target edits, or both, as compared to a construct comprising lacking the structural features. As used herein, the term “engineered” in reference to a guide RNA or polynucleotide encoding the same refers to a non-naturally occurring guide RNA or polynucleotide encoding the same. Such an engineered guide or engineered polynucleotide encoding an engineered guide, when administered to a subject, can be referred to as a heterologous guide RNA or heterologous polynucleotide. In some examples, the engineered guide RNA can be encoded by an engineered polynucleotide. In some instances, the engineered guide can be an RNA engineered guide. In some instances, the engineered guide can comprise RNA and can further comprise at least on deoxyribonucleotide. In some examples, the engineered guide RNA comprises a DNA base. In some examples, the engineered guide RNA comprises RNA bases exclusively. In some examples, the engineered guide RNA comprises modified DNA bases or unmodified DNA bases. In some examples, the engineered guide RNA comprises modified RNA bases or unmodified RNA bases. In some examples, the engineered guide RNA comprises both DNA and RNA bases. In some examples, an engineered guide of the disclosure can be utilized for RNA editing, for example to prevent or treat a disease or condition. In some cases, an engineered guide RNA can be used in association with a subject RNA editing entity to edit a target RNA or modulate expression of a polypeptide encoded by the target RNA. In some examples, compositions disclosed herein can include engineered guide RNAs capable of facilitating editing by subject RNA editing entities such as ADAR polypeptides or biologically active fragments thereof.

In some examples, provided herein are engineered latent guide RNAs that, upon hybridization to a target RNA implicated in a disease or condition, form a guide-target RNA scaffold comprising a structural feature selected from the group consisting of a bulge, an internal loop, a hairpin, and any combination thereof, wherein the structural feature substantially forms upon hybridization to the target RNA.

In some examples, an engineered guide RNA disclosed herein comprise: (a) at least one RNA editing enzyme recruiting domain; (b) at least one structural feature; or (c) any combination thereof, where the engineered guide RNA is configured to facilitate editing of a nucleotide base of a nucleotide of a target RNA molecule to modulate an expression level of a protein (e.g., ABCA4, APP, SERPINA1, HEXA, LRRK2, SNCA, CFTR, APP, GBA, PINK1 or LIPA) expressed from said target RNA molecule.

In some examples, chemical modification of the base of the nucleotide in the target RNA molecules (e.g., an adenosine to inosine edit) can be confirmed by sequencing (e.g., Sanger sequencing or next generating sequencing). In some examples, confirming that chemical modification has occurred comprises isolating one or more target RNA molecules to which an engineered guide has been administered and then converting the target RNA to cDNA by reverse transcriptase prior to sequencing. In some examples, the sequencing employed can be Sanger sequencing, next generation sequencing, or a combination thereof.

A. Targeting Domain

Engineered guide RNAs disclosed herein can be engineered in any way suitable for RNA editing. In some examples, an engineered guide RNA generally comprises at least a targeting sequence that allows it to hybridize to a region of a target RNA molecule. A targeting sequence can also be referred to as a “targeting domain” or a “targeting region”.

In some cases, a targeting sequence of an engineered guide RNA allows the engineered guide RNA to target an RNA sequence through base pairing, such as Watson Crick base pairing. In some examples, the targeting sequence can be located at either the N-terminus or C-terminus of the engineered guide RNA. In some cases, the targeting sequence can be located at both termini. The targeting sequence can be of any length. In some cases, the targeting sequence can be at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence can be no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered guide RNA comprises a targeting sequence that can be about 75-100, 80-110, 90-120, or 95-115 nucleotides in length. In some examples, an engineered guide comprises a targeting sequence that can be about 100 nucleotides in length.

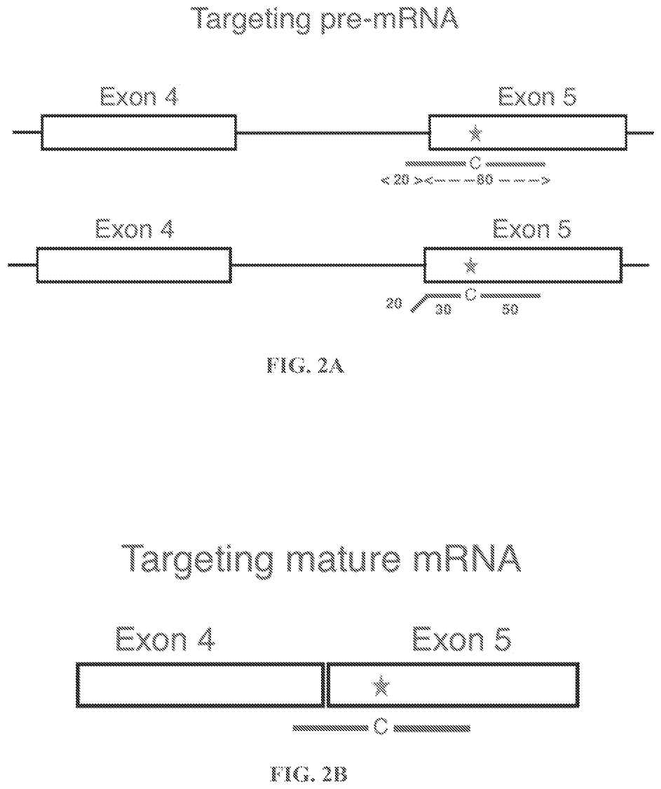

In some examples, the target RNA sequence can be an mRNA molecule or a pre-mRNA molecule. FIGS. 2A and 2B illustrate using engineered guide RNAs disclosed herein to target both pre-mRNA molecules (FIG. 2A) and mRNA molecules (FIG. 2B). As is illustrated in FIG. 2A, the engineered guide RNA is complementary, at least in part, to both an intron and an exon of a pre-mRNA molecule. In some examples, the engineered guide RNA can be complementary only to an exon region of a pre-mRNA molecule.

In some examples, the target RNA sequence can be an mRNA molecule. In some examples, the mRNA molecule comprises a premature stop codon. In some examples, the mRNA comprises 1, 2, 3, 4 or 5 premature stop codons. In some examples, the stop codon can be an amber stop codon (UAG), an ochre stop codon (UAA), or an opal stop codon (UGA), or a combination thereof. In some examples, the premature stop codon can be a consequence of a point mutation. In some examples, the premature stop codon causes translation termination of an expression product expressed by the mRNA molecule. In some examples, the premature stop codon can be produced by a point mutation on an mRNA molecule in combination with two additional nucleotides. In some examples, the two additional nucleotides can be (i) a U and (ii) an A or a G, on a 5′ and a 3′ end of the point mutation.

In some examples, the target RNA sequence can be a pre-mRNA molecule. In some examples, the pre-mRNA molecule comprises a splice site mutation. In some examples, the splice site mutation facilitates unintended splicing of a pre-mRNA molecule. In some examples, the splice site mutation results in mistranslation and/or truncation of a protein encoded by the pre-mRNA molecule.

In some examples, the target RNA molecule can be a pre-mRNA or mRNA molecule encoded by an ABCA4, APP, SERPINA1, HEXA, LRRK2, SNCA, CFTR, APP, GBA, PINK1 or LIPA gene, a fragment of any of these, or any combination thereof. In some examples, the target RNA molecule can be a encoded by gene selected from ABCA4, AAT, SERPINA1, SERPINA1 E342K, HEXA, LRRK2, SNCA, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. In some examples, the target RNA molecule encodes an ABCA4, APP, SERPINA1, HEXA, LRRK2, SNCA, CFTR, APP, GBA, PINK1, Tau, or LIPA protein, a fragment of any of these, or a combination thereof. In some examples, the target RNA molecule encodes ABCA4, AAT, SERPINA1, SERPINA1 E342K, HEXA, LRRK2, SNCA, APP, Tau, GBA, PINK1, RAB7A, CFTR, ALAS1, ATP7B, ATP7B G1226R, HFE C282Y, LIPA c.894 G>A, a fragment any of these, or any combination thereof. In some examples, the DNA encoding the RNA molecule comprises a mutation relative to an otherwise identical reference DNA molecule. In some examples, the RNA molecule comprises a mutation relative to an otherwise identical reference RNA molecule. In some examples, the protein encoded for by the target RNA molecule comprises a mutation relative to an otherwise identical reference protein.

In some examples, the target RNA molecule can be encoded by, at least in part, a SERPINA1 gene. In some examples, the SERPINA1 gene comprises a mutation. In some examples, the mutation can be a substitution of a G with an A at nucleotide position 9989 within a wildtype SERPINA1 gene (such as accession number NC_000014.9:c94390654-94376747). In some examples, the mutation causes or contributes to an antitrypsin (AAT) deficiency, such as alpha-1 antitrypsin deficiency (AATD) in a subject to which the engineered guide RNA can be administered to treat the AATD.

In some examples, the target RNA molecule can be encoded by, at least in part, an ABCA4 gene. In some examples, the ABCA4 gene comprises a mutation. In some examples, the mutation comprises a substitution of a G with an A at nucleotide position 5882 in a wildtype ABCA4 gene (such as accession number NC_000001.11:c94121149-93992837). In some examples, the mutation comprises a G with an A at nucleotide position 5714 in a wildtype ABCA4 gene (such as accession number NC_000001.11:c94121149-93992837). In some examples, the mutation comprises a substitution of a G with an A at nucleotide position 6320 in a wildtype ABCA4 gene (such as accession number NC_000001.11:c94121149-93992837). In some examples, the mutation causes or contributes to macular degeneration in a subject to which the engineered guide RNA is administered. In some examples, the macular degeneration can be Stargardt macular degeneration. In some examples the target RNA molecule comprises an adenosine with a 5′ G. In some examples, the adenosine with the 5′ G can be the base intended for chemical modification by the RNA editing entity. In some examples, the RNA editing entity can be an ADAR, and the ADAR chemically modifies the adenosine with the 5′ G after recruitment by the double stranded substrate.

In some examples, the target RNA molecule encodes, at least in part, an amyloid precursor protein (APP). In some examples, the target RNA molecule encodes, at least in part, an APP cleavage site. In some examples, the target RNA molecule encodes, at least in part, a beta secretase (BACE) or gamma secretase cleavage site of an APP protein. In some examples, the target RNA molecule encodes, at least in part, a beta secretase (BACE) cleavage site of an APP protein. In some examples, the target RNA molecule encodes, at least in part, an APP start site. In some examples, cleavage of the APP protein at the cleavage cite causes or contributes to Amyloid beta (AD or Abeta) peptide deposition in the brain or blood vessels. In some examples, the Abeta deposition causes or contributes to a neurodegenerative disease. In some examples, the disease comprises Alzheimer's disease, Parkinson's disease, corticobasal degeneration, dementia with Lewy bodies, Lewy body variant of Alzheimer's disease, Parkinson's disease with dementia, Pick's disease, progressive supranuclear palsy, dementia, fronto-temporal dementia with Parkinsonism linked to tau mutations on chromosome 17, or any combination thereof.

In some cases, a targeting sequence comprises at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence complementarity to a target RNA. In some cases, a targeting sequence comprises less than 100% complementarity to a target RNA sequence. For example, a targeting sequence and a region of a target RNA that is bound by the targeting sequence can have a single base mismatch. In other cases, the targeting sequence of an engineered guide RNA comprises at least about 1, 2, 3, 4, 5, 6, 7, 8, 9, 10, 11, 12, 13, 14, 15, 16, 20, 30, 40, 50, 60, 70, 80, 90, 100, 110, 120, 130, 140, 150, 160, 170, 180, 190, or up to about 200 unpaired bases, wherein the unpaired bases are apart of a structural feature disclosed herein. In other cases, the targeting sequence of an engineered guide RNA comprises no more than about 1, 2, 3, 4, 5, 6, 7, 8, 9, 10, 11, 12, 13, 14, 15, 16, 20, 30, 40, 50, 60, 70, 80, 90, 100, 110, 120, 130, 140, 150, 160, 170, 180, 190, or up to about 200 unpaired bases, wherein the unpaired bases are part of a structural feature disclosed herein. In some examples, unpaired bases are associated with structural features provided herein. In some examples, a targeting sequence comprises at least about 1, 2, 3, 4, 5, 6, 7, 8, 9, 10, 11, 12, 13, 14, or up to about 15 nucleotides that differ relative to an RNA sequence with perfect complementarity to a subject target RNA. In some examples, a targeting sequence comprises no more than about 1, 2, 3, 4, 5, 6, 7, 8, 9, 10, 11, 12, 13, 14, or 15 nucleotides that differ in complementarity from a wildtype RNA of a subject target RNA. In some cases, a targeting sequence comprises at least 50 nucleotides having complementarity to a target RNA. In some cases, a targeting sequence comprises from 50 to 150 nucleotides having complementarity to a target RNA. In some cases, a targeting sequence comprises from 50 to 200 nucleotides having complementarity to a target RNA. In some cases, a targeting sequence comprises from 50 to 250 nucleotides having complementarity to a target RNA. In some cases, a targeting sequence comprises from 50 to 300 nucleotides having complementarity to a target RNA. In some cases, a targeting sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides having complementarity to a target RNA. In some cases, a targeting sequence comprises more than 50 nucleotides total and has at least 50 nucleotides having complementarity to a target RNA. In some cases, a targeting sequence comprises from 50 to 400 nucleotides total and has from 50 to 150 nucleotides having complementarity to a target RNA. In some cases, a targeting sequence comprises from 50 to 400 nucleotides total and has from 50 to 200 nucleotides having complementarity to a target RNA. In some cases, a targeting sequence comprises from 50 to 400 nucleotides total and has from 50 to 250 nucleotides having complementarity to a target RNA. In some cases, a targeting sequence comprises from 50 to 400 nucleotides total and has from 50 to 300 nucleotides having complementarity to a target RNA. In some cases, the at least 50 nucleotides having complementarity to a target RNA are separated by a structural feature described herein (e.g. one or more mismatches, one or more bulges, or one or more loops, one or more hairpins, or any combination thereof). In some cases, the 50 to 150 nucleotides having complementarity to a target RNA are separated by a structural feature described herein (e.g. one or more mismatches, one or more bulges, or one or more loops, one or more hairpins, or any combination thereof). In some cases, the 50 to 200 nucleotides having complementarity to a target RNA are separated by a structural feature described herein (e.g. one or more mismatches, one or more bulges, or one or more loops, one or more hairpins, or any combination thereof). In some cases, the 50 to 250 nucleotides having complementarity to a target RNA are separated by a structural feature described herein (e.g. one or more mismatches, one or more bulges, or one or more loops, one or more hairpins, or any combination thereof). In some cases, the 50 to 300 nucleotides having complementarity to a target RNA are separated by a structural feature described herein (e.g. one or more mismatches, one or more bulges, or one or more loops, one or more hairpins, or any combination thereof). For example, a targeting sequence can comprise a total of 54 nucleotides wherein, sequentially, 25 nucleotides are complementarity to a target RNA, 4 nucleotides form a bulge, and 25 nucleotides are complementarity to a target RNA. As another example, a targeting sequence comprises a total of 118 nucleotides wherein, sequentially, 25 nucleotides are complementarity to a target RNA, 4 nucleotides form a bulge, 25 nucleotides are complementarity to a target RNA, 14 nucleotides form an internal loop, and 50 nucleotides are complementary to a target RNA.

In some cases, an engineered guide RNA can comprise multiple targeting sequences. In some instances, one or more target sequence domains in the engineered guide RNA can bind to one or more regions of a target RNA. For example, a first targeting sequence can be configured to be at least partially complementary to a first region of a target RNA (e.g., a first exon of a pre-mRNA), while a second targeting sequence can be configured to be at least partially complementary to a second region of a target RNA (e.g. a second exon of a pre-mRNA). In some instances, multiple target sequences can be operatively linked to provide continuous hybridization of multiple regions of a target RNA. In some instances, multiple target sequences can provide non-continuous hybridization of multiple regions of a target RNA. A “non-continuous” overlap or hybridization refers to hybridization of a first region of a target RNA by a first targeting sequence, along with hybridization of a second region of a target RNA by a second targeting sequence, where the first region and the second region of the target RNA are discontinuous (e.g., where there is intervening sequence between the first and the second region of the target RNA). For example, a targeting sequence can be configured to bind to a portion of a first exon and can comprise an internal asymmetric loop (e.g., an oligo tether) that is configured to bind to a portion of a second exon, while the intervening sequence between the portion of exon 1 and the portion of exon 2 is not hybridized by either the targeting sequence or the oligo tether. Use of an engineered guide RNA as described herein configured for non-continuous hybridization can provide a number of benefits. For instance, such a guide can potentially target pre-mRNA during transcription (or shortly thereafter), which can then facilitate chemical modification using a deaminase (e.g., ADAR) co-transcriptionally and thus increase the overall efficiency of the chemical modification. Further, the use of oligo tethers to provide non-continuous hybridization while skipping intervening sequence can result in shorter, more specific guide RNA with fewer off-target editing.

In some instances, an engineered guide RNA configured for non-continuous hybridization to a target RNA (e.g., an engineered guide RNA comprising a targeting sequence with an oligo tether) can be configured to bind distinct regions or a target RNA separated by intervening sequence. In some instances, the intervening sequence can be at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 nucleotides. In some instances, the targeting sequence and oligo tether can target distinct non-continuous regions of the same intron or exon. In some instances, the targeting sequence and oligo tether can target distinct non-continuous regions of adjacent exons or introns. In some instances, the targeting sequence and oligo tether can target distinct non-continuous regions of distal exons or introns.

B. Engineered Guide RNAs Having a Recruiting Domain

In some examples, an engineered guide RNA can comprise an RNA editing entity recruiting domain formed and present in the absence of binding to a target RNA. An RNA editing entity can be recruited by an RNA editing entity recruiting domain on an engineered guide RNA. In some examples, an engineered guide RNA comprising an RNA editing entity recruiting domain can be configured to facilitate editing of a base of a nucleotide of a polynucleotide of a region of a subject target RNA, modulation expression of a polypeptide encoded by the subject target RNA, or both. In some cases, an engineered guide RNA can be configured to facilitate an editing of a base of a nucleotide or polynucleotide of a region of an RNA by a subject RNA editing entity. In order to facilitate editing, an engineered guide RNA of the disclosure can recruit an RNA editing entity.

Various RNA editing entity recruiting domains can be utilized. In some examples, a recruiting domain comprises: Glutamate ionotropic receptor AMPA type subunit 2 (GluR2), APOBEC, MS2-bacteriophage-coat-protein-recruiting domain, Alu, a TALEN recruiting domain, a Zn-finger polypeptide recruiting domain, a mega-TAL recruiting domain, or a Cas13 recruiting domain, combinations thereof, or modified versions thereof. In some examples, more than one recruiting domain can be included in an engineered guide of the disclosure. In examples where a recruiting sequence is present, the recruiting sequence can be utilized to position the RNA editing entity to effectively react with a subject target RNA after the targeting sequence, for example an antisense sequence, hybridizes to a target RNA. In some cases, a recruiting sequence can allow for transient binding of the RNA editing entity to the engineered guide RNA. In some examples, the recruiting sequence allows for permanent binding of the RNA editing entity to the engineered guide. A recruiting sequence can be of any length. In some cases, a recruiting sequence can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting sequence can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting sequence can be about 45 nucleotides in length. In some cases, at least a portion of a recruiting sequence comprises at least 1 to about 75 nucleotides. In some cases, at least a portion of a recruiting sequence comprises about 45 nucleotides to about 60 nucleotides. In some aspects, an RNA editing entity recruiting domain can form a recruitment hairpin, as disclosed herein. A recruitment hairpin can recruit an RNA editing entity, such as ADAR. In some embodiments, a recruitment hairpin comprises a GluR2 domain. In some embodiments, a recruitment hairpin comprises an Alu domain.

In an embodiment, an RNA editing entity recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 3). In some cases, a recruiting domain can comprise at least about 80% sequence homology to at least about 10, 15, 20, 25, or 30 nucleotides of SEQ ID NO: 3. In some examples, a recruiting domain can comprise at least about 90%, 95%, 96%, 97%, 98%, or 99% sequence homology and/or length to SEQ ID NO: 3.

Additional RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In some examples, a recruiting domain can be from an MS2-bacteriophage-coat-protein-recruiting domain. In another embodiment, a recruiting domain can be from an Alu domain. In some examples, a recruiting domain can comprise at least about: 70%, 80%, 85%, 90%, or 95% sequence homology and/or length to at least about: 15, 20, 25, 30, or 35 nucleotides of an APOBEC, MS2-bacteriophage-coat-protein-recruiting domain, or Alu domain.

Any number of recruiting sequences can be found in an engineered guide RNA of the present disclosure. In some examples, at least about 0, 1, 2, 3, 4, 5, 6, 7, 8, 9, or up to about 10 recruiting sequences can be included in an engineered guide. Recruiting sequences can be located at any position of guide RNAs. In some cases, a recruiting sequence can be on an N-terminus, middle, or C-terminus of a polynucleotide. A recruiting sequence can be upstream or downstream of a targeting sequence. In some cases, a recruiting sequence flanks a targeting sequence of a guide RNA. A recruiting sequence can comprise all ribonucleotides or deoxyribonucleotides, although a recruiting sequence comprising both ribo- and deoxyribonucleotides may in some cases not be excluded.

In some examples, a double stranded RNA (dsRNA) substrate (a guide-target RNA scaffold) is formed upon hybridization of an engineered guide RNA of the present disclosure to a target RNA. In some examples, the target RNA forming the double stranded substrate comprises a portion of an mRNA molecule encoded by a SERPINA1 gene. In some examples the targeting region of the engineered guide forming the double stranded substrate is, at least in part, complementary to a portion of an mRNA molecule encoded by a SERPINA1 gene. In some examples the double stranded substrate comprises a single mismatch. In some examples, the mismatch comprised any two nucleotides that do not base pair. In some examples, the engineered guide RNA comprises an RNA editing entity recruiting domain that comprises a hairpin. In some examples, the hairpin comprises an ADAR recruiting domain. In some examples, the double stranded substrate can be formed by a target RNA comprising an mRNA encoded by the SERPINA1 gene and an engineered guide RNA complementary to a portion of the mRNA encoded by the SERPINA1 gene, wherein the double stranded substrate comprises a single mismatch and an RNA editing entity recruiting domain that comprises a hairpin.

In certain examples, the engineered guide RNA targeting SERPINA1 mRNA and having an RNA editing entity recruiting domain comprises a polynucleotide of the following sequence:

(SEQ ID NO: 4
AUGGGUAUGGCCUCUAAAAACAUGGCCCCAGCAGCUUCAGUCCCUUUCU
CGUCGAUGGUC [symbol] [symbol] [symbol]
AGCACAGCCUUAUGCACGGCCUUGGAGAGCUU
CAGGGGUG;

the C in bold and underlined text indicates the base that produces a mismatch with the target A to be edited; the GluR2 hairpin recruiting domain is indicated by bold, italicized, and underlined). In some examples, the engineered guide RNA comprises a polynucleotide having at least 99% identity, at least 95% identity, at least 90% identity, at least 85% identity, at least 80% identity, or at least 70% identity to the above SEQ ID NO: 4. In some examples, the engineered guide comprises a polynucleotide having at least 99% length, at least 95% length, at least 90% length, at least 85% length, at least 80% length, or at least 70% length to the above SEQ ID NO: 4.

In some examples, the target RNA forming the double stranded substrate (the guide-target RNA complex) comprises a portion of a pre-mRNA molecule encoded by a SERPINA1 gene. In some examples the targeting region of the engineered guide RNA forming the double stranded substrate is, at least in part, complementary to a portion of a pre-mRNA molecule encoded by the SERPINA1 gene. In some examples the double stranded substrate comprises a single mismatch. In some examples, the mismatch comprised any two nucleotides that do not base pair. In some examples, the engineered substrate comprises an RNA editing entity recruiting domain that comprises a hairpin. In some examples, the hairpin functions as an ADAR recruiting domain. In some examples, the double stranded substrate can be formed by a target RNA comprising a pre-mRNA encoded by the SERPINA1 gene and an engineered guide complementary to a portion of the pre-mRNA encoded by the SERPINA1 gene, wherein the double stranded substrate comprises a single mismatch and a hairpin.

In certain examples, an engineered guide RNA targeting SERPINA1 pre-mRNA and having an RNA editing entity recruiting domain comprises a polynucleotide of the following sequence:

(SEQ ID NO: 5
AUGGGUAUGGCCUCUAAAAACAUGGCCCCAGCAGCUUCAGUCCCUUUCU
CGUC [symbol] [symbol] [symbol] [symbol] [symbol]
AUGCACGGCcUggaggggagagaagCaga;

the C in bold and underlined text indicates the base that produces a mismatch with the target A to be edited; the GluR2 hairpin recruiting domain is indicated in bold, italicized, and underlined text). In some examples, the engineered guide comprises a polynucleotide having at least 99% identity, at least 95% identity, at least 90% identity, at least 85% identity, at least 80% identity, or at least 70% identity to the above SEQ ID NO: 5. In some examples, the engineered guide RNA comprises a polynucleotide having at least 99% length, at least 95% length, at least 90% length, at least 85% length, at least 80% length, or at least 70% length to the above SEQ ID NO: 5.

In some examples, the engineered guide RNAs disclosed herein comprise a polynucleotide having at least 99% identity, at least 95% identity, at least 90% identity, at least 85% identity, at least 80% identity, or at least 70% identity to the sequences listed below in TABLE 1. In some examples, the engineered guide RNA comprises a polynucleotide having at least 99% length, at least 95% length, at least 90% length, at least 85% length, at least 80% length, or at least 70% length to the sequences listed below in TABLE 1. In some examples the engineered guides disclosed herein comprises a polynucleotide of any of the sequences listed below in TABLE 1.

TABLE 1
Engineered Guide RNAs Having a Recruiting Domain

| SEQ ID NO | Sequence | Annotation |
|---|---|---|
| SEQ ID NO: 4 | AUGGGUAUGGCCUCUAAAAACAUGGCCCCAGC | SERPINA1_ |
| | AGCUUCAGUCCCUUUCUCGUCGAUGGUCCACCC | FlipIntGluR2 |
| | UAUGAUAUUGUUGUAAAUCGUAUAACAAUAUG | |
| | AUAAGGUGAGCACAGCCUUAUGCACGGCCUU | |
| | GAGAGCUUCAGGGGUG | |
| SEQ ID NO: 5 | AUGGGUAUGGCCUCUAAAAACAUGGCCCCAGC | FlipIntGluR2_ |
| | AGCUUCAGUCCCUUUCUCGUCGAUGGUCCACCC | preSERPINA1 |
| | UAUGAUAUUGUUGUAAAUCGUAUAACAAUAUG | |
| | AUAAGGUGAGCACAGCCUUAUGCACGGCcUggag | |
| | gggagagaagcaga | |

C. Engineered Guides with Latent Structure

In some embodiments, the present disclosure provides for engineered guide RNAs with latent structure, also referred to as “latent guide RNAs”. Latent structure refers to a structural feature that forms only upon hybridization of a guide RNA to a target RNA, within the guide-target RNA scaffold. For example, the sequence of a guide RNA provides one or more latent structural features, but these latent structural features only form upon hybridization of the latent guide RNA to the target RNA. Thus, the one or more latent structural features manifest as structural features upon hybridization of the latent guide RNA to the target RNA. Upon hybridization of the latent guide RNA to the target RNA, the structural feature is formed and the latent structure provided in the guide RNA is, thus, unmasked. An engineered latent guide RNA can comprise a portion of sequence that, upon hybridization to a target RNA, forms at least a portion of a structural feature, other than a single A/C mismatch feature at the target adenosine to be edited. In some embodiments, a latent structural feature formed upon hybridization to a target RNA includes at least two contiguous nucleotides of the guide RNA. In some instances, a latent structural feature can include any structural feature disclosed herein in addition to an A/C mismatch at the target adenosine to be edited, with these additional structural features providing an increase in an amount of editing of the target adenosine by an RNA editing entity, a decrease in an amount of editing of local off-target adenosines by an RNA editing entity, or both relative to an otherwise comparable guide RNA lacking the additional structural features. Thus, the engineered latent guide RNAs of the present disclosure comprise latent structural features that manifest more than one structural feature upon hybridization to a target RNA within the guide-target RNA scaffold. The presence of multiple structural features within the guide-target RNA scaffold provides for secondary and tertiary, three-dimensional structures that serve as superior substrates for ADAR and drive unexpectedly high editing efficiency of the target adenosine and highly selective editing of the target adenosine (reduced editing of local off-target adenosines) by an otherwise promiscuous enzyme. The latent structures of the engineered latent guide RNAs described herein, which substantially form structural features upon hybridization to a target RNA within the guide-target RNA scaffold can also, upon editing by ADAR, drive improved translation and increased protein production. In some embodiments, the engineered guide RNAs disclosed herein having latent structure can be administered to a cell and result in superior on-target editing, reduced local off-target editing, increased translation, increased protein production, or any combination thereof, all in comparison to a guide RNA lacking latent structures. In some embodiments, the engineered latent guide RNAs disclosed herein have latent structure and also lack an RNA editing entity recruiting domain that is formed and present in the absence of binding to the target RNA. A double stranded RNA (dsRNA) substrate can also be referred to herein as a guide-target RNA scaffold. A guide-target RNA scaffold, as disclosed herein, can be a resulting double stranded RNA duplex formed upon hybridization of a guide RNA to a target RNA, where the guide RNA prior to hybridizing to the target RNA comprise a portion of sequence that, upon hybridization to a target RNA, forms at least a portion of a structural feature, other than a single A/C mismatch feature at the target adenosine to be edited. Accordingly, a guide-target RNA scaffold has structural features formed within the double stranded RNA duplex. For example, the guide-target RNA scaffold can have two or more features selected from a bulge, mismatch, internal loop, hairpin, or wobble base pair. In some embodiments, engineered guide RNAs with latent structure lack an RNA editing entity recruiting domain that is formed and present in the absence of binding to the target RNA. In some embodiments, engineered guide RNAs with latent structure further comprise a recruiting domain that is formed and present in the absence of binding to the target RNA.

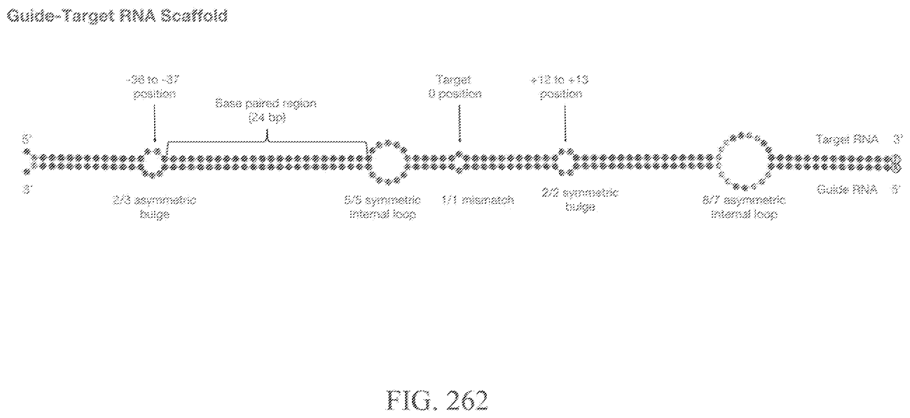

FIG. 262 shows a legend of various exemplary structural features of the present disclosure present in guide-target RNA scaffolds formed upon hybridization of a latent guide RNA of the present disclosure to a target RNA. Example structural features shown in FIG. 262 include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side). Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the 2/3 asymmetric bulge in this legend is at the −36 to −37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a 2/3 asymmetric bulge at position −36, then the 2/3 asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position +12, then the 2/2 symmetric bulge forms from the +12 to the +13 position with respect to the target nucleotide to be edited (position 0) on the target RNA side of the guide-target RNA scaffold.

In some examples, an engineered guide RNA disclosed herein, when present in an aqueous solution and not bound to the target RNA molecule, does not recruit an RNA editing entity. In some examples, (i) the engineered guide RNA, when present in an aqueous solution and not bound to the target RNA molecule, does not comprise any bulges, internal loops, or hairpins; (ii) the engineered guide RNA, when present in an aqueous solution and not bound to the target RNA molecule, does not comprise any bulges, internal loops, or hairpins that recruit a human ADAR1 with a dissociation constant lower than about 100 nM, 200 nM, 300 nM, 400 nM, 500 nM, 600 nM, 700 nM, 800 nM, 900 nM, or 1,000 nM as determined by an in vitro assay; (iii) the engineered guide RNA, upon at least partially binding to the target RNA molecule and thereby forming a guide-target RNA scaffold, is configured to adopt a structural feature (along with the target RNA) that recruits an RNA editing entity; or (iv) any combination thereof. In some examples, the engineered guide RNA, when present in an aqueous solution and not bound to the target RNA molecule, if it binds to the RNA editing entity, does so with a dissociation constant of about greater than or equal to about 100 nM, 200 nM, 300 nM, 400 nM, 500 nM, 600 nM, 700 nM, 800 nM, 900 nM, or 1,000 nM. In some examples, the engineered guide RNA, when present in an aqueous solution and not bound to the target RNA molecule, if it binds to the RNA editing entity, does so with a dissociation constant of about greater than or equal to about 500 nM. In some examples, the engineered guide RNAs disclosed herein, when present in an aqueous solution and not bound to the target RNA molecule, lack a structural feature described herein. In some examples, the engineered guide RNAs disclosed herein, when present in an aqueous solution and not bound to the target RNA molecule does not comprise any bulges, internal loops, or hairpins. In some examples, the engineered guide RNAs disclosed herein, when present in an aqueous solution and not bound to the target RNA molecule, may be linear and do not comprise any structural features.

In some examples, an engineered guide RNA can be configured to facilitate an editing of a base of a nucleotide or polynucleotide of a region of a target RNA by a subject RNA editing entity. In order to facilitate editing, an engineered guide RNA of the disclosure can recruit an RNA editing entity.

In cases where an RNA editing entity recruiting domain formed and present in the absence of binding to a target RNA is not included in an engineered guide RNA, the engineered guide RNA can be still capable of associating with a subject RNA editing entity (e.g., ADAR) to facilitate editing of a target RNA and/or modulate expression of a polypeptide encoded by a subject target RNA. This can be achieved through the presence of structural features that manifest from latent structures formed upon hybridization of the guide RNA and target RNA. Structural features can comprise any one of a: mismatch, symmetrical bulge, asymmetrical bulge, symmetrical internal loop, asymmetrical internal loop, hairpins, wobble base pairs, a structured motif, circularized RNA, chemical modification, or any combination thereof. In an aspect, a double stranded RNA (dsRNA) substrate (a guide-target RNA scaffold), can be formed upon hybridization of an engineered guide RNA of the present disclosure to a target RNA. Described herein is a feature, which corresponds to one of several structural features that can be present in a dsRNA substrate of the present disclosure. Examples of features include a mismatch, a bulge (symmetrical bulge or asymmetrical bulge), an internal loop (symmetrical internal loop or asymmetrical internal loop), or a hairpin (a hairpin comprising a non-targeting domain). Engineered guide RNAs of the present disclosure can have from 1 to 50 features. For example, engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 5 to 25, from 5 to 30, from 5 to 35, from 5 to 40, from 5 to 45, from 5 to 50, from 1 to 2, from 1 to 3, from 1 to 4, from 1 to 5, from 1 to 6, from 1 to 7, from 1 to 8, from 1 to 9, from 1 to 10, from 1 to 11, from 1 to 12, from 1 to 13, from 1 to 14, from 1 to 15, from 1 to 16, from 1 to 17, from 1 to 18, from 1 to 19, from 1 to 20, from 1 to 21, from 1 to 22, from 1 to 23, from 1 to 24, from 1 to 25, from 1 to 26, from 1 to 27, from 1 to 28, from 1 to 29, from 1 to 30, from 1 to 31, from 1 to 32, from 1 to 33, from 1 to 34, from 1 to 35, from 1 to 36, from 1 to 37, from 1 to 38, from 1 to 39, from 1 to 40, from 1 to 41, from 1 to 42, from 1 to 43, from 1 to 44, from 1 to 45, from 1 to 46, from 1 to 47, from 1 to 48, from 1 to 49, from 4 to 5, from 2 to 10, from 20 to 40, from 10 to 40, from 20 to 50, from 30 to 50, from 4 to 7, or from 8 to 10 features. In some instances, an engineered guide RNA can have at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 features.

As disclosed herein, a “structured motif” comprises two or more features in a dsRNA substrate (a guide-target RNA scaffold).

A double stranded RNA (dsRNA) substrate (a guide-target RNA scaffold) is formed upon hybridization of an engineered guide RNA of the present disclosure to a target RNA. As disclosed herein, a “mismatch” refers to a nucleotide in a guide RNA that can be unpaired or fully unpaired to an opposing nucleotide in a target RNA within the dsRNA. A mismatch can comprise any two nucleotides that do not base pair, are not complementary, or both. In some embodiments, a mismatch can be an A/C mismatch. An A/C mismatch can comprise a C in an engineered guide RNA of the present disclosure opposite an A in a target RNA. An A/C mismatch can comprise an A in an engineered guide RNA of the present disclosure opposite an C in a target RNA. In an embodiment, a G/G mismatch can comprise a G in an engineered guide RNA of the present disclosure opposite a G in a target RNA. In some embodiments, a mismatch positioned 5′ of the edit site can facilitate base-flipping of the target A to be edited. A mismatch can also help confer sequence specificity. In an embodiment, a mismatch comprises a G/G mismatch. In an embodiment, a mismatch comprises an A/C mismatch, wherein the A can be in the target RNA and the C can be in the targeting sequence of the engineered guide RNA. In another embodiment, the A in the A/C mismatch can be the base of the nucleotide in the target RNA edited by a subject RNA editing entity.

In an aspect, a structural feature can include both latent structures as described above, as well as non-latent structures. As described herein, a “non-latent structure” refers to a structure that can form in an engineered RNA independent of binding to a target RNA. For example, a recruitment hairpin (e.g., the GluR2 recruitment domain) may not be a latent structure, but rather may form in an engineered RNA independently. In some cases, a structural feature can form when an engineered RNA binds to a target RNA and is, thus, latent structure. A structural feature can also form when an engineered RNA associates with other molecules such as a peptide, a nucleotide, or a small molecule. In certain embodiments, a structural feature is present when an engineered guide RNA is in association with a target RNA.

In some examples, a structural feature is present when an engineered guide RNA is in association with a target RNA. A structural feature of an engineered guide RNA can form a substantially linear two-dimensional structure. A structural feature of an engineered guide RNA can comprise a linear region, a stem-loop, a cruciform, a toe hold, a mismatch bulge, or any combination thereof. In some instances, a structural feature can comprise a stem, a hairpin loop, a pseudoknot, a bulge, an internal loop, a multiloop, a G-quadruplex, or any combination thereof. In some examples, an engineered guide RNA can adopt an A-form, a B-form, a Z-form, or any combination thereof.

In some cases, a structural feature can be a hairpin. In some cases, an engineered guide RNA can lack a hairpin domain (for instance, the engineered guide RNA does not form an intramolecular hairpin in the absence of hybridization to a target RNA). In other cases, an engineered guide RNA can contain a hairpin domain or more than one hairpin domain. A hairpin can be located anywhere in a guide RNA. As disclosed herein, a “hairpin” is an RNA duplex wherein a single RNA strand has folded in upon itself to form the RNA duplex. The single RNA strand folds upon itself due to having nucleotide sequences upstream and downstream of the folding region base pairs to each other. A hairpin can have from 10 to 500 nucleotides in length of the entire duplex structure. The stem-loop structure of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can be a recruitment hairpin or a non-recruitment hairpin. A hairpin can be located anywhere within the engineered guide RNAs of the present disclosure. In some embodiments, one or more hairpins can be present at the 3′ end of an engineered guide RNAs of the present disclosure, at the 5′ end of an engineered guide RNAs of the present disclosure or within the targeting sequence of an engineered guide RNAs of the present disclosure, or any combination thereof.

In yet another aspect, a structural feature comprises a non-recruitment hairpin. A non-recruitment hairpin, as disclosed herein, can exhibit functionality that improves localization of the engineered guide RNAs to the target RNA. In some embodiments, a non-recruitment hairpin exhibits functionality that improves localization of the engineered guide RNAs to the region of the target RNA for hybridization. In some embodiments, the non-recruitment hairpin improves nuclear retention. In some embodiments, structural features are not formed from latent structures and are, instead, pre-formed structures (e.g., a GluR2 recruitment hairpin or a hairpin from U7 snRNA). In some embodiments, a non-recruitment hairpin that is preformed (is not a latent structure) is a hairpin from U7 snRNA.

In another aspect, a structural feature comprises a wobble base. A “wobble base pair” refers to two bases that weakly pair. For example, a wobble base pair of the present disclosure can refer to a G paired with a U.

A hairpin of the present disclosure can be of any length. In an aspect, a hairpin can be from about 5-200 or more nucleotides. In some cases, a hairpin can comprise about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 or more nucleotides. In other cases, a hairpin can also comprise 5 to 10, 5 to 20, 5 to 30, 5 to 40, 5 to 50, 5 to 60, 5 to 70, 5 to 80, 5 to 90, 5 to 100, 5 to 110, 5 to 120, 5 to 130, 5 to 140, 5 to 150, 5 to 160, 5 to 170, 5 to 180, 5 to 190, 5 to 200, 5 to 210, 5 to 220, 5 to 230, 5 to 240, 5 to 250, 5 to 260, 5 to 270, 5 to 280, 5 to 290, 5 to 300, 5 to 310, 5 to 320, 5 to 330, 5 to 340, 5 to 350, 5 to 360, 5 to 370, 5 to 380, 5 to 390, or 5 to 400 nucleotides.

In some cases, a structural feature can be a bulge. A bulge can comprise 1 to 4 (intentional) nucleic acid mismatch(s) between the target strand and an engineered guide RNA strand. In some cases, 1 to 4 consecutive mismatch(s) between strands constitutes a bulge as long as the bulge region, mismatched stretch of nucleotides, is flanked on both sides with hybridized, complementary dsRNA regions. A bulge can be located at any location of a guide RNA other than the last nucleotides of either the 5′ end or the 3′ end. In some cases, a bulge is be located from about 30 to about 70 nucleotides from a 5′ hydroxyl or the 3′ hydroxyl.

In an embodiment, a double stranded RNA (dsRNA) substrate (guide-target RNA scaffold) is formed upon hybridization of an engineered guide RNA of the present disclosure to a target RNA. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A. In some embodiments, selective editing of the target A is achieved by positioning the target A between two bulges (e.g., positioned between a 5′ end bulge and a 3′ end bulge, based on the engineered guide RNA). In some embodiments, the two bulges are both symmetrical bulges. In some embodiments, the two bulges each are formed by 2 nucleotides on the engineered guide RNA side of the guide-RNA scaffold and 2 nucleotides on the target RNA side of the guide-RNA scaffold. In some embodiments, the two bulges each are formed by 3 nucleotides on the engineered guide RNA side of the guide-RNA scaffold and 3 nucleotides on the target RNA side of the guide-RNA scaffold. In some embodiments, the two bulges each are formed by 4 nucleotides on the engineered guide RNA side of the guide-RNA scaffold and 4 nucleotides on the target RNA side of the guide-RNA scaffold. In some embodiments, the target A is position between the two bulges, and is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides from a bulge (e.g., from a 5′ end bulge or a 3′ end bulge). In some embodiments, additional structural features are located between the bulges (e.g., between the 5′ end bulge and the 3′ end bulge). In some embodiments, a mismatch in a bulge comprises a nucleotide base for editing in the target RNA (e.g., an A/C mismatch in the bulge, wherein part of the bulge in the engineered guide RNA comprises a C mismatched to an A in the part of the bulge in the target RNA, and the A is edited).

In an aspect, a double stranded RNA (dsRNA) substrate (guide-target RNA scaffold) is formed upon hybridization of an engineered guide RNA of the present disclosure to a target RNA. A bulge can be a symmetrical bulge or an asymmetrical bulge. For illustrative purposes, examples of a symmetrical bulge and an asymmetrical bulge in a guide-target RNA scaffold are depicted in FIG. 262. In FIG. 262, an example of a 2/2 symmetric bulge (2 nucleotides on the target RNA side and 2 nucleotides on the guide RNA side) at the +12 to +13 position is shown. In FIG. 262, an example of a 2/3 asymmetric bulge (2 nucleotides on the target RNA side and 3 nucleotides on the guide RNA side) at position −36 to −37 is also shown. A symmetrical bulge is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

In some cases, a double stranded RNA (dsRNA) substrate (guide-target RNA scaffold) can be formed upon hybridization of an engineered guide RNA of the present disclosure to a target RNA. A bulge can be a symmetrical bulge or an asymmetrical bulge. An asymmetrical bulge is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

In an aspect, a double stranded RNA (dsRNA) substrate (guide-target RNA scaffold) can be formed upon hybridization of an engineered guide RNA of the present disclosure to a target RNA. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. For illustrative purposes, examples of a symmetrical bulge and an asymmetrical bulge in a guide-target RNA scaffold are depicted in FIG. 262. In FIG. 262, an example of an 8/7 asymmetric internal loop (8 nucleotides on the target RNA side and 7 nucleotides on the guide RNA side) at the +31 to +38 position is shown. In FIG. 262, an example of a 5/5 symmetric internal loop (5 nucleotides on the target RNA side and 5 nucleotides on the guide RNA side) at position −7 to −11 is also shown. Internal loops present in the vicinity of the edit site can help with base flipping of the target A in the target RNA to be edited.

An internal loop can be a symmetrical internal loop or an asymmetrical internal loop. In some embodiments, selective editing of the target A is achieved by positioning the target A between two loops (e.g., positioned between a 5′ end loop and a 3′ end loop, based on the engineered guide RNA). In some embodiments, the two loops are both symmetrical loops. In some embodiments, the two loops each are formed by 5 nucleotides on the engineered guide RNA side of the guide-target RNA scaffold and 5 nucleotides on the target RNA side of the guide-target RNA scaffold. In some embodiments, the two loops each are formed by 6 nucleotides on the engineered guide RNA side of the guide-target RNA scaffold and 6 nucleotides on the target RNA side of the guide-target RNA scaffold. In some embodiments, the two loops each are formed by 7 nucleotides on the engineered guide RNA side of the guide-target RNA scaffold and 7 nucleotides on the target RNA side of the guide-target RNA scaffold. In some embodiments, the two loops each are formed by 8 nucleotides on the engineered guide RNA side of the guide-target RNA scaffold and 8 nucleotides on the target RNA side of the guide-target RNA scaffold. In some embodiments, the two loops each are formed by 9 nucleotides on the engineered guide RNA side of the guide-target RNA scaffold and 9 nucleotides on the target RNA side of the guide-target RNA scaffold. In some embodiments, the two loops each are formed by 10 nucleotides on the engineered guide RNA side of the guide-target RNA scaffold and 10 nucleotides on the target RNA side of the guide-target RNA scaffold. In some embodiments, the target A is position between the two loops, and is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides from a loop (e.g., from a 5′ end loop or a 3′ end loop). In some embodiments, additional structural features are located between the loops (e.g., between the 5′ end loop and the 3′ end loop).

A symmetrical internal loop is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered guide RNA side of the guide-target RNA scaffold target and 15 nucleotides on the target RNA side of the guide-target RNA scaffold. A symmetrical internal loop of the present disclosure can be formed by 20 nucleotides on the engineered guide RNA side of the guide-target RNA scaffold target and 20 nucleotides on the target RNA side of the guide-target RNA scaffold. A symmetrical internal loop of the present disclosure can be formed by 30 nucleotides on the engineered guide RNA side of the guide-target RNA scaffold target and 30 nucleotides on the target RNA side of the guide-target RNA scaffold. A symmetrical internal loop of the present disclosure can be formed by 40 nucleotides on the engineered guide RNA side of the guide-target RNA scaffold target and 40 nucleotides on the target RNA side of the guide-target RNA scaffold. A symmetrical internal loop of the present disclosure can be formed by 50 nucleotides on the engineered guide RNA side of the guide-target RNA scaffold target and 50 nucleotides on the target RNA side of the guide-target RNA scaffold. A symmetrical internal loop of the present disclosure can be formed by 60 nucleotides on the engineered guide RNA side of the guide-target RNA scaffold target and 60 nucleotides on the target RNA side of the guide-target RNA scaffold. A symmetrical internal loop of the present disclosure can be formed by 70 nucleotides on the engineered guide RNA side of the guide-target RNA scaffold target and 70 nucleotides on the target RNA side of the guide-target RNA scaffold. A symmetrical internal loop of the present disclosure can be formed by 80 nucleotides on the engineered guide RNA side of the guide-target RNA scaffold target and 80 nucleotides on the target RNA side of the guide-target RNA scaffold. A symmetrical internal loop of the present disclosure can be formed by 90 nucleotides on the engineered guide RNA side of the guide-target RNA scaffold target and 90 nucleotides on the target RNA side of the guide-target RNA scaffold. A symmetrical internal loop of the present disclosure can be formed by 100 nucleotides on the engineered guide RNA side of the guide-target RNA scaffold target and 100 nucleotides on the target RNA side of the guide-target RNA scaffold. A symmetrical internal loop of the present disclosure can be formed by 110 nucleotides on the engineered guide RNA side of the guide-target RNA scaffold target and 110 nucleotides on the target RNA side of the guide-target RNA scaffold. A symmetrical internal loop of the present disclosure can be formed by 120 nucleotides on the engineered guide RNA side of the guide-target RNA scaffold target and 120 nucleotides on the target RNA side of the guide-target RNA scaffold. A symmetrical internal loop of the present disclosure can be formed by 130 nucleotides on the engineered guide RNA side of the guide-target RNA scaffold target and 130 nucleotides on the target RNA side of the guide-target RNA scaffold. A symmetrical internal loop of the present disclosure can be formed by 140 nucleotides on the engineered guide RNA side of the guide-target RNA scaffold target and 140 nucleotides on the target RNA side of the guide-target RNA scaffold. A symmetrical internal loop of the present disclosure can be formed by 150 nucleotides on the engineered guide RNA side of the guide-target RNA scaffold target and 150 nucleotides on the target RNA side of the guide-target RNA scaffold. A symmetrical internal loop of the present disclosure can be formed by 200 nucleotides on the engineered guide RNA side of the guide-target RNA scaffold target and 200 nucleotides on the target RNA side of the guide-target RNA scaffold. A symmetrical internal loop of the present disclosure can be formed by 250 nucleotides on the engineered guide RNA side of the guide-target RNA scaffold target and 250 nucleotides on the target RNA side of the guide-target RNA scaffold. A symmetrical internal loop of the present disclosure can be formed by 300 nucleotides on the engineered guide RNA side of the guide-target RNA scaffold target and 300 nucleotides on the target RNA side of the guide-target RNA scaffold. A symmetrical internal loop of the present disclosure can be formed by 350 nucleotides on the engineered guide RNA side of the guide-target RNA scaffold target and 350 nucleotides on the target RNA side of the guide-target RNA scaffold. A symmetrical internal loop of the present disclosure can be formed by 400 nucleotides on the engineered guide RNA side of the guide-target RNA scaffold target and 400 nucleotides on the target RNA side of the guide-target RNA scaffold. A symmetrical internal loop of the present disclosure can be formed by 450 nucleotides on the engineered guide RNA side of the guide-target RNA scaffold target and 450 nucleotides on the target RNA side of the guide-target RNA scaffold. A symmetrical internal loop of the present disclosure can be formed by 500 nucleotides on the engineered guide RNA side of the guide-target RNA scaffold target and 500 nucleotides on the target RNA side of the guide-target RNA scaffold. A symmetrical internal loop of the present disclosure can be formed by 600 nucleotides on the engineered guide RNA side of the guide-target RNA scaffold target and 600 nucleotides on the target RNA side of the guide-target RNA scaffold. A symmetrical internal loop of the present disclosure can be formed by 700 nucleotides on the engineered guide RNA side of the guide-target RNA scaffold target and 700 nucleotides on the target RNA side of the guide-target RNA scaffold. A symmetrical internal loop of the present disclosure can be formed by 800 nucleotides on the engineered guide RNA side of the guide-target RNA scaffold target and 800 nucleotides on the target RNA side of the guide-target RNA scaffold. A symmetrical internal loop of the present disclosure can be formed by 900 nucleotides on the engineered guide RNA side of the guide-target RNA scaffold target and 900 nucleotides on the target RNA side of the guide-target RNA scaffold. A symmetrical internal loop of the present disclosure can be formed by 1000 nucleotides on the engineered guide RNA side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

An asymmetrical internal loop is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered guide RNA side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered guide RNA side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered guide RNA side of the guide-target RNA scaffold. An asymmetrical internal loop of the present disclosure can be formed by 5 nucleotides on the target RNA side of the guide-target RNA scaffold and 100 nucleotides on the engineered guide RNA side of the guide-target RNA scaffold. An asymmetrical internal loop of the present disclosure can be formed by 5 nucleotides on the target RNA side of the guide-target RNA scaffold and 150 nucleotides on the engineered guide RNA side of the guide-target RNA scaffold. An asymmetrical internal loop of the present disclosure can be formed by 5 nucleotides on the target RNA side of the guide-target RNA scaffold and 200 nucleotides on the engineered guide RNA side of the guide-target RNA scaffold. An asymmetrical internal loop of the present disclosure can be formed by 5 nucleotides on the target RNA side of the guide-target RNA scaffold and 300 nucleotides on the engineered guide RNA side of the guide-target RNA scaffold. An asymmetrical internal loop of the present disclosure can be formed by 5 nucleotides on the target RNA side of the guide-target RNA scaffold and 400 nucleotides on the engineered guide RNA side of the guide-target RNA scaffold. An asymmetrical internal loop of the present disclosure can be formed by 5 nucleotides on the target RNA side of the guide-target RNA scaffold and 500 nucleotides on the engineered guide RNA side of the guide-target RNA scaffold. An asymmetrical internal loop of the present disclosure can be formed by 5 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 5 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 5 nucleotides on the engineered guide RNA side of the guide-target RNA scaffold. An asymmetrical internal loop of the present disclosure can be formed by 300 nucleotides on the target RNA side of the guide-target RNA scaffold and 5 nucleotides on the engineered guide RNA side of the guide-target RNA scaffold. An asymmetrical internal loop of the present disclosure can be formed by 200 nucleotides on the target RNA side of the guide-target RNA scaffold and 5 nucleotides on the engineered guide RNA side of the guide-target RNA scaffold. An asymmetrical internal loop of the present disclosure can be formed by 150 nucleotides on the target RNA side of the guide-target RNA scaffold and 5 nucleotides on the engineered guide RNA side of the guide-target RNA scaffold. An asymmetrical internal loop of the present disclosure can be formed by 100 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 100 nucleotides on the engineered guide RNA side of the guide-target RNA scaffold. An asymmetrical internal loop of the present disclosure can be formed by 50 nucleotides on the target RNA side of the guide-target RNA scaffold and 150 nucleotides on the engineered guide RNA side of the guide-target RNA scaffold. An asymmetrical internal loop of the present disclosure can be formed by 50 nucleotides on the target RNA side of the guide-target RNA scaffold and 200 nucleotides on the engineered guide RNA side of the guide-target RNA scaffold. An asymmetrical internal loop of the present disclosure can be formed by 50 nucleotides on the target RNA side of the guide-target RNA scaffold and 300 nucleotides on the engineered guide RNA side of the guide-target RNA scaffold. An asymmetrical internal loop of the present disclosure can be formed by 50 nucleotides on the target RNA side of the guide-target RNA scaffold and 400 nucleotides on the engineered guide RNA side of the guide-target RNA scaffold. An asymmetrical internal loop of the present disclosure can be formed by 50 nucleotides on the target RNA side of the guide-target RNA scaffold and 500 nucleotides on the engineered guide RNA side of the guide-target RNA scaffold. An asymmetrical internal loop of the present disclosure can be formed by 50 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 50 nucleotides on the engineered guide RNA side of the guide-target RNA scaffold. An asymmetrical internal loop of the present disclosure can be formed by 500 nucleotides on the target RNA side of the guide-target RNA scaffold and 50 nucleotides on the engineered guide RNA side of the guide-target RNA scaffold. An asymmetrical internal loop of the present disclosure can be formed by 400 nucleotides on the target RNA side of the guide-target RNA scaffold and 50 nucleotides on the engineered guide RNA side of the guide-target RNA scaffold. An asymmetrical internal loop of the present disclosure can be formed by 300 nucleotides on the target RNA side of the guide-target RNA scaffold and 50 nucleotides on the engineered guide RNA side of the guide-target RNA scaffold. An asymmetrical internal loop of the present disclosure can be formed by 200 nucleotides on the target RNA side of the guide-target RNA scaffold and 50 nucleotides on the engineered guide RNA side of the guide-target RNA scaffold. An asymmetrical internal loop of the present disclosure can be formed by 150 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 150 nucleotides on the engineered guide RNA side of the guide-target RNA scaffold. An asymmetrical internal loop of the present disclosure can be formed by 100 nucleotides on the target RNA side of the guide-target RNA scaffold and 200 nucleotides on the engineered guide RNA side of the guide-target RNA scaffold. An asymmetrical internal loop of the present disclosure can be formed by 100 nucleotides on the target RNA side of the guide-target RNA scaffold and 300 nucleotides on the engineered guide RNA side of the guide-target RNA scaffold. An asymmetrical internal loop of the present disclosure can be formed by 100 nucleotides on the target RNA side of the guide-target RNA scaffold and 400 nucleotides on the engineered guide RNA side of the guide-target RNA scaffold. An asymmetrical internal loop of the present disclosure can be formed by 100 nucleotides on the target RNA side of the guide-target RNA scaffold and 500 nucleotides on the engineered guide RNA side of the guide-target RNA scaffold. An asymmetrical internal loop of the present disclosure can be formed by 100 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 100 nucleotides on the engineered guide RNA side of the guide-target RNA scaffold. An asymmetrical internal loop of the present disclosure can be formed by 500 nucleotides on the target RNA side of the guide-target RNA scaffold and 100 nucleotides on the engineered guide RNA side of the guide-target RNA scaffold. An asymmetrical internal loop of the present disclosure can be formed by 400 nucleotides on the target RNA side of the guide-target RNA scaffold and 100 nucleotides on the engineered guide RNA side of the guide-target RNA scaffold. An asymmetrical internal loop of the present disclosure can be formed by 300 nucleotides on the target RNA side of the guide-target RNA scaffold and 100 nucleotides on the engineered guide RNA side of the guide-target RNA scaffold. An asymmetrical internal loop of the present disclosure can be formed by 200 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 200 nucleotides on the engineered guide RNA side of the guide-target RNA scaffold. An asymmetrical internal loop of the present disclosure can be formed by 150 nucleotides on the target RNA side of the guide-target RNA scaffold and 300 nucleotides on the engineered guide RNA side of the guide-target RNA scaffold. An asymmetrical internal loop of the present disclosure can be formed by 150 nucleotides on the target RNA side of the guide-target RNA scaffold and 400 nucleotides on the engineered guide RNA side of the guide-target RNA scaffold. An asymmetrical internal loop of the present disclosure can be formed by 150 nucleotides on the target RNA side of the guide-target RNA scaffold and 500 nucleotides on the engineered guide RNA side of the guide-target RNA scaffold. An asymmetrical internal loop of the present disclosure can be formed by 150 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 150 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 150 nucleotides on the engineered guide RNA side of the guide-target RNA scaffold. An asymmetrical internal loop of the present disclosure can be formed by 300 nucleotides on the target RNA side of the guide-target RNA scaffold and 150 nucleotides on the engineered guide RNA side of the guide-target RNA scaffold. An asymmetrical internal loop of the present disclosure can be formed by 200 nucleotides on the target RNA side of the guide-target RNA scaffold and 300 nucleotides on the engineered guide RNA side of the guide-target RNA scaffold. An asymmetrical internal loop of the present disclosure can be formed by 200 nucleotides on the target RNA side of the guide-target RNA scaffold and 400 nucleotides on the engineered guide RNA side of the guide-target RNA scaffold. An asymmetrical internal loop of the present disclosure can be formed by 200 nucleotides on the target RNA side of the guide-target RNA scaffold and 500 nucleotides on the engineered guide RNA side of the guide-target RNA scaffold. An asymmetrical internal loop of the present disclosure can be formed by 200 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 200 nucleotides on the engineered guide RNA side of the guide-target RNA scaffold. An asymmetrical internal loop of the present disclosure can be formed by 500 nucleotides on the target RNA side of the guide-target RNA scaffold and 200 nucleotides on the engineered guide RNA side of the guide-target RNA scaffold. An asymmetrical internal loop of the present disclosure can be formed by 400 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 400 nucleotides on the engineered guide RNA side of the guide-target RNA scaffold. An asymmetrical internal loop of the present disclosure can be formed by 300 nucleotides on the target RNA side of the guide-target RNA scaffold and 500 nucleotides on the engineered guide RNA side of the guide-target RNA scaffold. An asymmetrical internal loop of the present disclosure can be formed by 300 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 300 nucleotides on the engineered guide RNA side of the guide-target RNA scaffold. An asymmetrical internal loop of the present disclosure can be formed by 500 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 500 nucleotides on the engineered guide RNA side of the guide-target RNA scaffold. An asymmetrical internal loop of the present disclosure can be formed by 400 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 500 nucleotides on the engineered guide RNA side of the guide-target RNA scaffold. Thus, an asymmetrical internal loop can be a structural feature formed from latent structure provided by an engineered latent guide RNA.

Structural features that comprise an internal loop can be of any size greater than 5 nucleotides. In some cases, an internal loop comprise at least: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 600, 700, 800, 900, or 1000 nucleotides. In some cases, an internal loop comprise at least about 5-10, 5-15, 10-20, 15-25, 20-30, 5-30, 5-40, 5-50, 5-60, 5-70, 5-80, 5-90, 5-100, 5-110, 5-120, 5-130, 5-140, 5-150, 5-200, 5-250, 5-300, 5-350, 5-400, 5-450, 5-500, 5-600, 5-700, 5-800, 5-900, 5-1000, 20-50, 20-60, 20-70, 20-80, 20-90, 20-100, 20-110, 20-120, 20-130, 20-140, 20-150, 30-40, 30-50, 30-60, 30-70, 30-80, 30-90, 30-100, 30-110, 30-120, 30-130, 30-140, 30-150, 30-200, 30-250, 30-300, 30-350, 30-400, 30-450, 30-500, 30-600, 30-700, 30-800, 30-900, 30-1000, 40-50, 40-60, 40-70, 40-80, 40-90, 40-100, 40-110, 40-120, 40-130, 40-140, 40-150, 40-200, 40-250, 40-300, 40-350, 40-400, 40-450, 40-500, 40-600, 40-700, 40-800, 40-900, 40-1000, 50-60, 50-70, 50-80, 50-90, 50-100, 50-110, 50-120, 50-130, 50-140, 50-150, 50-200, 50-250, 50-300, 50-350, 50-400, 50-450, 50-500, 50-600, 50-700, 50-800, 50-900, 50-1000, 60-70, 60-80, 60-90, 60-100, 60-110, 60-120, 60-130, 60-140, 60-150, 60-200, 60-250, 60-300, 60-350, 60-400, 60-450, 60-500, 60-600, 60-700, 60-800, 60-900, 60-1000, 70-80, 70-90, 70-100, 70-110, 70-120, 70-130, 70-140, 70-150, 70-200, 70-250, 70-300, 70-350, 70-400, 70-450, 70-500, 70-600, 70-700, 70-800, 70-900, 70-1000, 80-90, 80-100, 80-110, 80-120, 80-130, 80-140, 80-150, 80-200, 80-250, 80-300, 80-350, 80-400, 80-450, 80-500, 80-600, 80-700, 80-800, 80-900, 80-1000, 90-100, 90-110, 90-120, 90-130, 90-140, 90-150, 90-200, 90-250, 90-300, 90-350, 90-400, 90-450, 90-500, 90-600, 90-700, 90-800, 90-900, 90-1000, 100-110, 100-120, 100-130, 100-140, 100-150, 100-200, 100-250, 100-300, 100-350, 100-400, 100-450, 100-500, 100-600, 100-700, 100-800, 100-900, 100-1000, 110-120, 110-130, 110-140, 110-150, 110-200, 110-250, 110-300, 110-350, 110-400, 110-450, 110-500, 110-600, 110-700, 110-800, 110-900, 110-1000, 120-130, 120-140, 120-150, 120-200, 120-250, 120-300, 120-350, 120-400, 120-450, 120-500, 120-600, 120-700, 120-800, 120-900, 120-1000, 130-140, 130-150, 130-200, 130-250, 130-300, 130-350, 130-400, 130-450, 130-500, 130-600, 130-700, 130-800, 130-900, 130-1000, 140-150, 140-200, 140-250, 140-300, 140-350, 140-400, 140-450, 140-500, 140-600, 140-700, 140-800, 140-900, 140-1000, 150-200, 150-250, 150-300, 150-350, 150-400, 150-450, 150-500, 150-600, 150-700, 150-800, 150-900, 150-1000, 200-250, 200-300, 200-350, 200-400, 200-450, 200-500, 200-600, 200-700, 200-800, 200-900, 200-1000, 250-300, 250-350, 250-400, 250-450, 250-500, 250-600, 250-700, 250-800, 250-900, 250-1000, 300-350, 300-400, 300-450, 300-500, 300-600, 300-700, 300-800, 300-900, 300-1000, 350-400, 350-450, 350-500, 350-600, 350-700, 350-800, 350-900, 350-1000, 400-450, 400-500, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, or 900-1000 nucleotides in total.

In some embodiments, a double stranded RNA (dsRNA) substrate (a guide-target RNA scaffold) comprises a base paired region. As disclosed herein, a base paired (bp) region refers to a stretch of the guide-target RNA scaffold in which the bases in the guide RNA are paired with opposing bases in the target RNA. Base paired regions can extend from one end of the guide-target RNA scaffold to the other end of the guide-target RNA scaffold. Base paired regions can extend between two structural features. Base paired regions can extend from one end of the guide-target RNA scaffold to a structural feature. Base paired regions can extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

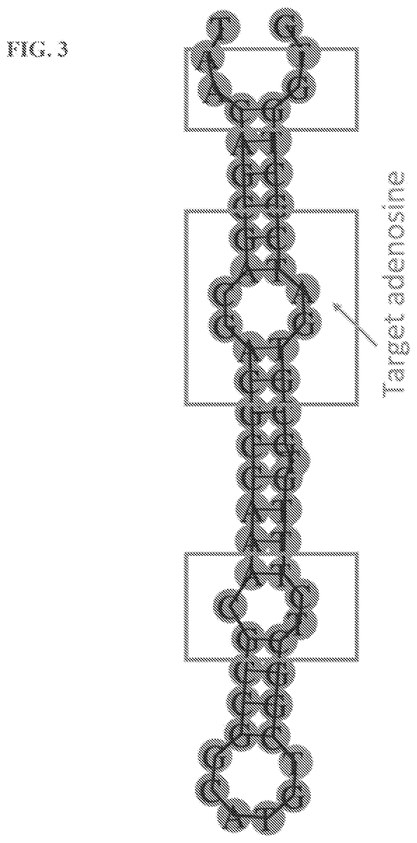

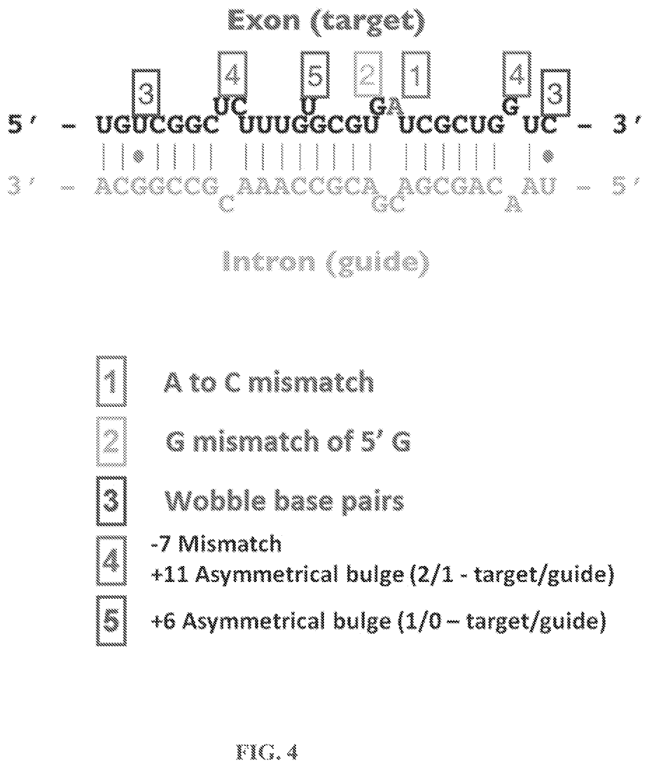

In some examples, a double stranded RNA (dsRNA) substrate (a guide-target RNA scaffold) is formed upon hybridization of an engineered guide of the present disclosure to a target RNA. In some examples, the double stranded substrate comprises structural features mimicking the structural features of a naturally occurring ADAR substrate. In some examples, the naturally occurring ADAR substrate can be a drosophila ADAR substrate. In some examples, the naturally occurring drosophila ADAR substrate can be as depicted in FIGS. 3 and 4 and comprises two bulges. The specific nucleotide interactions forming the structural features of the drosophila substrate are annotated on the sequences listed in FIG. 4 and include (1) an A to C mismatch, (2) a G mismatch of a 5′G, (3) two wobble base pairs, (4) a mismatch at the −7 position and an asymmetrical bulge at the +11 position (2/1-target/guide), and (5) an asymmetrical bulge at the +6 position (1/0—target/guide). In some examples, the structural features of the double stranded substrate mimic the structural features of a drosophila substrate in that the double stranded substrate comprises one or more (e.g., 1, 2, 3, 4, 5, 6 or 7) of the structural features also present in the drosophila substrate. In some examples, the one or more structural features in the double stranded substrate share at least 70%, 80%, 85%, 90%, 95%, 98%, 99% or 100% sequence homology and/or length with one or more (e.g., 1, 2, 3, 4, 5, 6, or 7) structural features of the naturally occurring drosophila substrate. In some examples, the one or more structural features in the double stranded substrate share no sequence homology or less than 50% sequence homology with one or more structural features of the drosophila substrate. In some examples, the one or more features in the double stranded substrate can be positioned (relative to each other) the same or similarly as the structural features of the natural ADAR substrate.

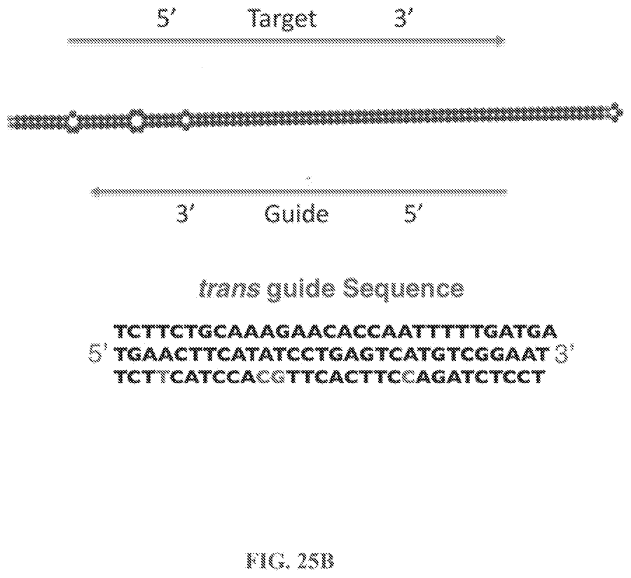

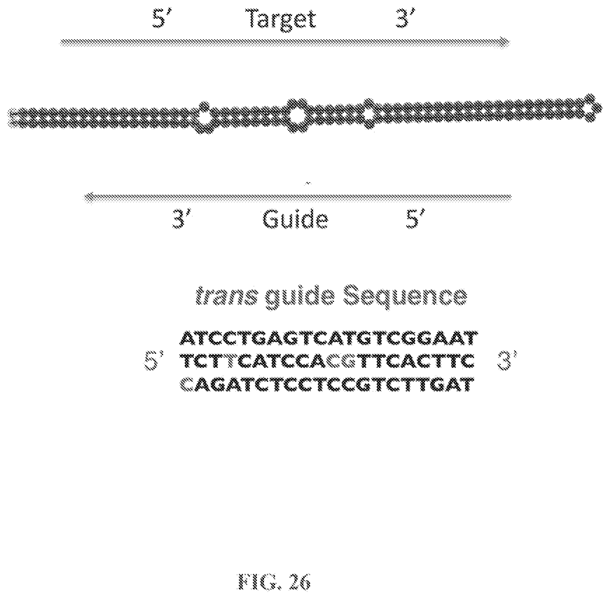

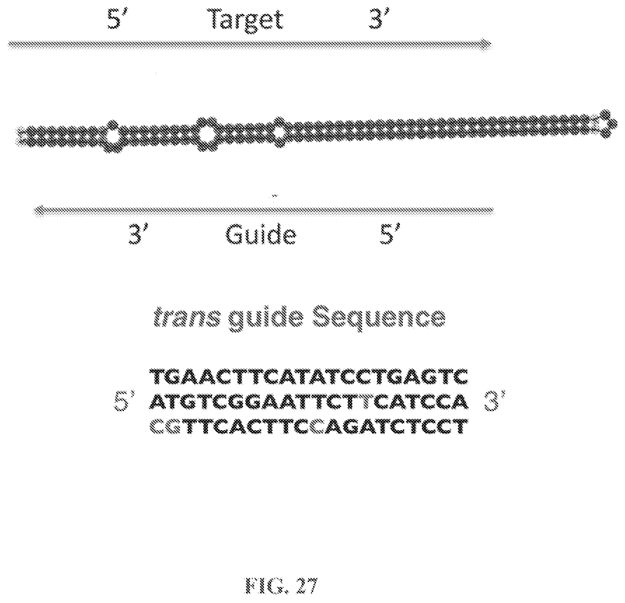

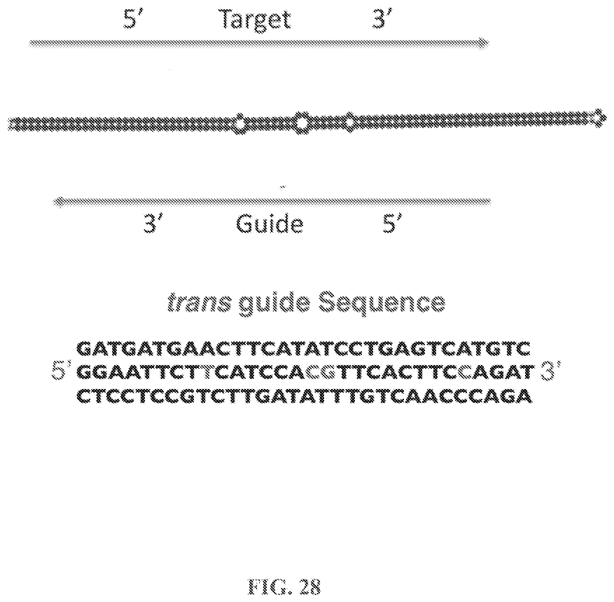

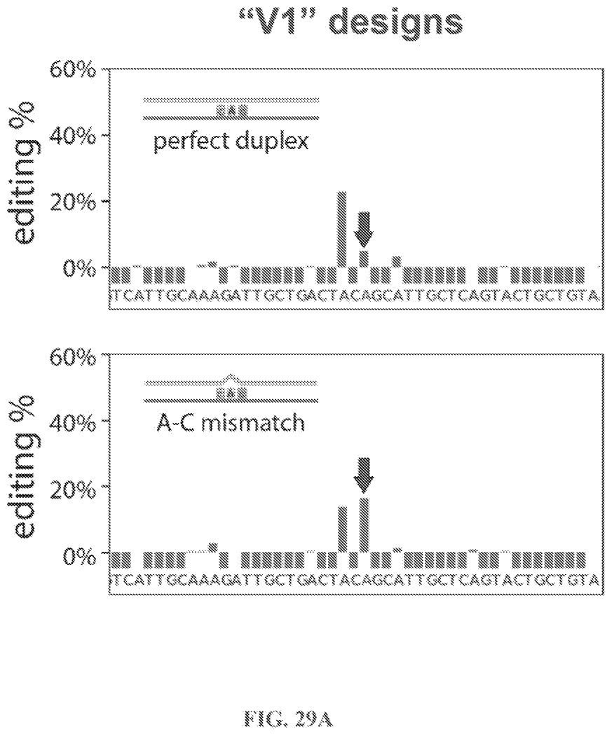

Some examples of mimicry and related features are included in FIG. 25A to FIG. 28.

In some cases, a structural feature can be a structured motif. As disclosed herein, a structured motif comprises two or more structural features in a dsRNA substrate. A structured motif can comprise any combination of structural features, such as in the above claims, to generate an ideal substrate for ADAR editing at a precise location(s). These structural motifs could be artificially engineered to maximized ADAR editing, and/or these structural motifs can be modeled to recapitulate known ADAR substrates.

In some cases, an engineered guide RNA can be circularized. In some cases, an engineered guide RNA provided herein can be circularized or in a circular configuration. In some aspects, an at least partially circular guide RNA lacks a 5′ hydroxyl or a 3′ hydroxyl.

In some examples, an engineered guide RNA can comprise a backbone comprising a plurality of sugar and phosphate moieties covalently linked together. In some examples, a backbone of an engineered guide RNA can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered guide RNA can lack a 5′ reducing hydroxyl, a 3′ reducing hydroxyl, or both, capable of being exposed to a solvent. In some embodiments, a backbone of an engineered guide can lack a 5′ reducing hydroxyl, a 3′ reducing hydroxyl, or both, capable of being exposed to nucleases. In some embodiments, a backbone of an engineered guide can lack a 5′ reducing hydroxyl, a 3′ reducing hydroxyl, or both, capable of being exposed to hydrolytic enzymes. In some instances, a backbone of an engineered guide can be represented as a polynucleotide sequence in a circular 2-dimensional format with one nucleotide after the other. In some instances, a backbone of an engineered guide can be represented as a polynucleotide sequence in a looped 2-dimensional format with one nucleotide after the other. In some cases, a 5′ hydroxyl, a 3′ hydroxyl, or both, can be joined through a phosphorus-oxygen bond. In some cases, a 5′ hydroxyl, a 3′ hydroxyl, or both, can be modified into a phosphoester with a phosphorus-containing moiety.

In some embodiments, the present disclosure provides for split guide RNA systems, where an engineered guide RNA of the present disclosure comprising a recruiting domain (e.g., GluR2) may be delivered as a split guide RNA system.

In some embodiments, a split guide RNA system can comprise two segments—an ADAR recruiting domain (e.g., GluR2 or Alu) and at least one targeting domain. The targeting domain can be at the 5′ and/or 3′ end of the recruiting domain. At least one targeting domain has a sequence that is only partially complementary to the sequence of segment of the target RNA. Binding of the two segments to the target RNA forms a trimolecular complex which recruits ADAR enzymes to deaminate one or more mismatched adenosine residues in the guide-target RNA scaffold.

In some embodiments, a split guide RNA system can comprise two segments—a first segment comprising a first portion of a recruiting domain (e.g., GluR2 or Alu) and, optionally, a part of a targeting domain and a second segment comprising a second portion of the recruiting domain and optionally, a part of a targeting domain. For example, a recruiting domain (e.g., a GluR2 hairpin) may be placed internally, within the targeting domain. The internal recruiting domain can be split into two asymmetric 5′ and 3′ segments, with the 5′ GluR2 segment located within the first guide RNA and the 3′ GluR2 segment located within the second guide RNA. Upon hybridization of the two segments of the engineered guide RNA to the target RNA, the GluR2 hairpin is re-constituted. The binding of the two segments to the target RNA, thus, forms a trimolecular complex, which contains a reconstituted GluR2 hairpin capable of recruiting ADAR for target-specific RNA-editing.

In some embodiments, an engineered guide RNA described herein can comprise modifications. A modification can be a substitution, insertion, deletion, chemical modification, physical modification, stabilization, purification, or any combination thereof. In some cases, a modification can be a chemical modification. Suitable chemical modifications comprise any one of. 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3phosphorothioate or any combinations thereof. In some embodiments, an engineered guide RNA described herein does not comprise modifications.

### Guide RNA Selection by High Throughput Guide Screening Assay

In some embodiments, an engineered guide RNA can be selected by a high throughput guide screening assay. A high throughput guide screening assay for selecting engineered guide RNAs was completed with ABCA4, LRRK2, and Serpina1 target RNA and the results are shown in TABLE 2. TABLE 2 shows the disease associated with the target RNA, the tissue expression pattern of the target RNA, the in vivo ADAR type used in the editing, the target motif in the target RNA, the target nucleotide in the target motif for the target RNA, the codon change with a successful editing of the target nucleotide, the associated amino acid change in the protein encoded by the edited target RNA, and the total number of guide RNA designs screened in the high throughput assay are shown for each target RNA.

TABLE 2
Summary of Targets

| | | | | | | Amino | |
|---|---|---|---|---|---|---|---|
| Target | | | | | Codon | acid | # gRNA |
| RNA | Disease | Tissue | ADAR1/2 | Motif | change | change | designs |
| ABCA4 | Stargardt | Ocular/ | ADAR1* | GAA | GAA −> | E −> G | 2,688 |
| | Disease | Photoreceptors/ | | | GGA | | |
| | | RPE | | | | | |
| LRRK2 | Parkinson's | CNS | ADAR2 | CAG | AGC −> | S −> G | 2,536 |
| | Disease | | | | GGC | | |
| SERPINA1 | Alpha-1- | Liver | ADAR1 | CAA | AAG −> | K −> E | 1,824 |
| | Antitrypsin | | | | GAG | | |
| | Deficiency | | | | | | |

*indicates that the ADAR type is predicted

### RNA-Editing Entities

In some examples, the guide-target RNA scaffold produced upon hybridization of the guide RNA and target RNA recruits an RNA editing entity. In some examples, an RNA editing entity comprises an ADAR. In some examples, an ADAR comprises any one of: ADAR1, ADAR1p110, ADAR1p150, ADAR2, ADAR3, APOBEC protein, or any combination thereof. In some examples, the ADAR RNA editing entity can be ADAR1. In some examples, additionally, or alternatively, the ADAR RNA editing entity can be ADAR2. In some examples, additionally, or alternatively, the ADAR RNA editing entity can be ADAR3. In an aspect, an RNA editing entity can be a non-ADAR. In some examples, the RNA editing entity can be an APOBEC protein. In some examples, the RNA editing entity can be APOBEC1, APOBEC2, APOBEC3A, APOBEC3B, APOBEC3C, APOBEC3E, APOBEC3F, APOBEC3G, APOBEC3H, APOBEC4, or any combination thereof. In some examples, the ADAR or APOBEC can be mammalian. In some examples, the ADAR or APOBEC protein can be human. In some examples, the ADAR or APOBEC protein can be recombinant (e.g., an exogenously delivered recombinant ADAR or APOBEC protein), modified (e.g., an exogenously delivered modified ADAR or APOBEC protein), endogenous, or any combination thereof. In some examples, the RNA editing entity can be a fusion protein. In some examples, the RNA editing entity can be a functional portion of an RNA editing entity, such as any of the RNA editing proteins provided herein. In some instances, an RNA editing entity can comprise at least about 70% sequence homology and/or length to APOBEC1, APOBEC2, ADAR1, ADAR1p110, ADAR1p150, ADAR2, ADAR3, or any combination thereof.

Other RNA editing entities are also contemplated. In some examples, the RNA editing entity comprises a clustered regularly interspaced short palindromic repeats (CRISPR) system. In some cases, an RNA editing entity can be a virus-encoded RNA-dependent RNA polymerase. In some cases, an RNA editing entity can be a virus-encoded RNA-dependent RNA polymerase from measles, mumps, or parainfluenza. In some instances, an RNA editing entity can be an enzyme from Trypanosoma brucei capable of adding or deleting a nucleotide or nucleotides in a target RNA. In some instances, an RNA editing entity can be an enzyme from Trypanosoma brucei capable of adding or deleting an Uracil or more than one Uracil in a target RNA. In some instances, an RNA editing entity comprises a recombinant enzyme. In some cases, an RNA editing entity comprises a fusion polypeptide. In some cases, an RNA editing entity does not comprise a fusion polypeptide.

### Therapeutic Applications

Disclosed herein are methods of delivering any engineered guide disclosed herein (e.g., an engineered guide, a vector encoding or comprising an engineered guide, and any pharmaceutical formulations thereof) to a cell. In some examples, methods of delivering an engineered guide to a cell comprise delivering directly or indirectly to the cell an engineered guide that at least partially hybridizes to and forms, at least in part, a double stranded substrate with at least a portion of a target RNA molecule, wherein the double stranded substrate comprises at least one structural feature, and wherein the double stranded substrate recruits an RNA editing entity and facilitates a chemical modification of a base of a nucleotide in the target RNA molecule by the RNA editing entity. In some examples, the chemical modification of the base of the nucleotide in the target RNA molecules can be confirmed by sequencing. In some examples, confirming that chemical modification has occurred comprises isolating one or more target RNA molecules to which an engineered guide has been administered and then converting the target RNA to cDNA by reverse transcriptase prior to sequencing. In some examples, the sequencing employed can be Sanger sequencing, next generation sequencing, or a combination thereof. In some examples, in any of the methods disclosed herein, the engineered guide can be encoded by a polynucleotide or a vector disclosed herein or can be comprised in a composition, pharmaceutical composition, isolated cell, or plurality of cells disclosed herein.

Also disclosed herein are methods of treating a disease or condition in a subject in need thereof comprising administering to the subject any engineered guide (e.g., an engineered guide, a vector encoding or comprising an engineered guide) disclosed herein. In some examples, the methods of treating or preventing a disease or a condition in a subject in need thereof comprise administering to the subject having the disease or the condition an engineered guide, thereby treating or preventing the disease or the condition in the subject, wherein the engineered guide: (a) at least in part associates with at least a portion of a target RNA molecule; (b) in association with the target RNA molecule, forms a double stranded substrate comprising at least one structural feature, and wherein the double stranded substrate recruits an RNA editing entity; and (c) facilitates a chemical modification of a base of a nucleotide in the target RNA molecule by the RNA editing entity. In some examples, chemical modification of the base can be confirmed by sequencing. In some examples, confirming that chemical modification has occurred comprises isolating one or more target RNA molecules to which an engineered guide has been administered and then converting the target RNA to cDNA by reverse transcriptase prior to sequencing. In some examples, the sequencing employed can be Sanger sequencing, next generation sequencing, or a combination thereof. In some examples, in any of the methods disclosed herein, the engineered guide can be encoded by a polynucleotide or a vector disclosed herein or can be comprised in a composition, pharmaceutical composition, isolated cell, or plurality of cells disclosed herein.

Compositions and methods provided herein can be utilized to modulate expression of a target. Modulation can refer to altering the expression of a gene or portion thereof at one of various stages, with a view to alleviate a disease or condition associated with the gene or a mutation in the gene. Modulation can be mediated at the level of transcription or post-transcriptionally. Modulating transcription can correct aberrant expression of splice variants generated by a mutation in a gene. In some cases, compositions and methods provided herein can be utilized to regulate gene translation of a target. Modulation can refer to decreasing or knocking down the expression of a gene or portion thereof by decreasing the abundance of a transcript. The decreasing the abundance of a transcript can be mediated by decreasing the processing, splicing, turnover or stability of the transcript; or by decreasing the accessibility of the transcript by translational machinery such as ribosome. In some cases, an engineered guide described herein can facilitate a knockdown. A knockdown can reduce the expression of a target RNA. In some cases, a knockdown can be accompanied by editing of an mRNA. In some cases, a knockdown can occur with substantially little to no editing of an mRNA. In some instances, a knockdown can occur by targeting an untranslated region of the target RNA, such as a 3′ UTR, a 5′ UTR or both. In some cases, a knockdown can occur by targeting a coding region of the target RNA. In some instances, a knockdown can be mediated by an RNA editing enzyme (e.g., ADAR). In some instances, an RNA editing enzyme can cause a knockdown by hydrolytic deamination of multiple adenosines in an RNA. Hydrolytic deamination of multiple adenosines in an RNA can be referred to as hyper-editing. In some cases, hyper-editing can occur in cis (e.g. in an Alu element) or in trans (e.g. in a target RNA by an engineered guide).). In some instances, an RNA editing enzyme can cause a knockdown by editing a target RNA to comprise a premature stop codon or prevent initiation of translation of the target RNA due to an edit in the target RNA.

In some examples, the disease or condition can be associated with a mutation in a DNA molecule or RNA molecule encoding ABCA4, APP, SERPINA1, HEXA, LRRK2, SNCA, CFTR, or LIPA, a fragment of any of these, or any combination thereof. In some examples, a protein encoded for by a mutated DNA molecule or RNA molecule encoding ABCA4, APP, SERPINA1, HEXA, LRRK2, SNCA, CFTR, or LIPA contributes to, at least in part, the pathogenesis or progression of a disease. In some examples, the disease or condition can be associated with a mutation in a DNA molecule or RNA molecule encoding ABCA4, AAT, SERPINA1, SERPINA1 E342K, HEXA, LRRK2, SNCA, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. In some examples, a protein encoded for by a mutated DNA molecule or RNA molecule encoding ABCA4, AAT, SERPINA1, SERPINA1 E342K, HEXA, LRRK2, SNCA, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof contributes to, at least in part, the pathogenesis or progression of a disease. In some examples, the mutation in the DNA or RNA molecule can be relative to an otherwise identical reference DNA or RNA molecule. In some examples, the mutation in the DNA or RNA molecule can be relative to an otherwise identical reference DNA or RNA molecule.

SERPINA1. In some embodiments, the present disclosure provides compositions and methods of use thereof of guide RNAs that are capable of facilitating RNA editing of serpin family A member 1 (SERPINA1). In some examples, the disease or condition can be an AAT deficiency or an associated lung or liver pathology (e.g., chronic obstructive pulmonary disease, cirrhosis, hepatocellular carcinoma) caused, at least in part, by a mutation in a SERPINA1 gene. In some examples, the mutation can be a substitution of a G with an A at nucleotide position 9989 within a wildtype SERPINA1 gene (such as accession number NC_000011:c94121149-93992837). In some examples, administration of the engineered guides disclosed herein restores expression of a normal AAT protein (e.g., as compared to an inactive or defective AAT protein) in a subject with an AAT deficiency. In some examples, a double stranded RNA (dsRNA) substrate (a guide-target RNA scaffold) is formed upon hybridization of an engineered guide of the present disclosure to a target RNA. In some examples, the target RNA forming the double stranded substrate comprises a portion of an mRNA or pre-mRNA molecule encoded by the SERPINA1 gene. In some examples the targeting region of the engineered guide forming the double stranded substrate is, at least in part, complementary to a portion of an mRNA or pre-mRNA molecule encoded by the SERPINA1 gene. In some examples the double stranded substrate comprises a single mismatch. In some examples, the engineered substrate additionally comprises one or two bulges. In some examples, the double stranded substrate can be formed by a target RNA comprising an mRNA or pre-mRNA encoded by the SERPINA1 gene and an engineered guide complementary to a portion of the mRNA encoded by the SERPINA1 gene, wherein the engineered substrate comprises a single mismatch. In some examples, the double stranded substrate can be formed by a target RNA comprising an mRNA or pre-mRNA encoded by the SERPINA1 gene and an engineered guide complementary to a portion of the mRNA or pre-mRNA encoded by the SERPINA1 gene, wherein the engineered substrate comprises a single mismatch, and wherein the engineered substrate comprises two additional bulges.

Guide RNAs can facilitate correction of a G to A mutation at nucleotide position 9989 of a SERPINA1 gene. In some embodiments, a guide RNA of the present disclosure can target, for example, E342K of SERPINA1. Said guide RNAs targeting a site in SERPINA1 can be encoded for by an engineered polynucleotide construct of the present disclosure. An engineered guide RNA targeting SERPINA1 can comprise a polynucleotide of any of the following sequences recited in TABLE 3:

TABLE 3
Engineered Guide RNAs or Polynucleotide Sequences Encoding Engineered
Guide RNAs against SERPINA1

| SEQ ID NO | Sequence | Structural Features |
|---|---|---|
| SEQ ID NO: 6 | AUGGGUAUGGCCUCUAAAAACAUGGCCCCAGCAGCU | 1 A/C mismatch |
| | UCAGUCCCUUUCUCGUCGAUGGUCAGCACAGCCUUA | |
| | UGCACGGCCUUGGAGAGCUUCAGGGGUG | |
| SEQ ID NO: 7 | ATGGGTATGGCCTCTAAAAACATGGCCCCAGCAGCTT | 1 A/C mismatch |
| | CAGTCATACCTTTCTCGTCGATGGTCAGCATGACAGCC | 2 3/3 symmetrical |
| | TTATGCACGGCCTTGGAGAGCTTCAGGGGTG | bulges |
| SEQ ID NO: 8 | AUGGGUAUGGCCUCUAAAAACAUGGCCCCAGCAGCU | 1 A/C mismatch |
| | UCAGUCCCUUUCUCGUCGAUGGUCAGCACAGCCUUA | |
| | UGCACGGCcUggaggggagagaagcaga | |
| SEQ ID NO: 9 | AUGGGUAUGGCCUCUAAAAACAUGGCCCCAGCAGCU | 1 A/C mismatch |
| | UCAGUCCCGGUCUCGUCGAUGGUCAGCACAGCCGUA | 1 A/G mismatch |
| | UGCACGGCcUggaggggagagaagcaga | |
| SEQ ID NO: 10 | AUGGGUAUGGCCUCUAAAAACAUGGCCCCAGCAGCU | 1 A/C mismatch |
| | UCAGUCAUACCUUUCUCGUCGAUGGUCAGCAUGACA | 2 3/3 symmetrical |
| | GCCUUAUGCACGGCcUggaggggagaGaagcaga | bulge |

The C in bold and italicized text indicates the base that produces a mismatch with the target A to be edited, the nucleotide sequence which form the additional bulges in the double stranded substrate are underlined, and the lowercase text signifies regions of the guide that hybridize to intronic target pre-mRNA. Further, a guide RNA targeting SERPINA1 can comprise any one of SEQ ID NO: 102-SEQ ID NO: 103 or SEQ ID NO: 297-SEQ ID NO: 327. In some examples, the engineered guide (including a latent guide RNA having latent structure) comprises a polynucleotide having at least 99% identity, at least 95% identity, at least 90% identity, at least 85% identity, at least 80% identity, or at least 70% identity to any one of SEQ ID NOS: 6-10, 102-103 or 297-327. In some examples, the engineered guide (including a latent guide RNA having latent structure) comprises a polynucleotide having at least 99% length, at least 95% length, at least 90% length, at least 85% length, at least 80% length, or at least 70% length to the above SEQ ID NOS: 6-10, 102-103 or 297-327. In some examples, hybridization of a latent guide RNA targeting SERPINA1 to a target SERPINA1 mRNA produces a guide-target RNA scaffold that comprises a structural features selected from the group consisting of: (i) one or more X₁/X₂ bulges, wherein X₁ is the number of nucleotides of the target RNA in the bulge and X₂ is the number of nucleotides of the engineered guide RNA in the bulge, and wherein the bulge is a 0/2 asymmetric bulge, a 0/3 asymmetric bulge, a 1/0 asymmetric bulge, a 2/0 asymmetric bulge, a 2/2 symmetric bulge, a 3/0 asymmetric bulge, a 2/2 symmetric bulge, or a 3/3 symmetric bulge; (ii) an X₁/X₂ internal loop, wherein X₁ is the number of nucleotides of the target RNA in the internal loop and X₂ is the number of nucleotides of the engineered guide RNA in the internal loop, and wherein the internal loop is a 5/5 symmetric internal loop; (iii) one or more mismatches, wherein the one or more mismatches is an A/C mismatch, an A/A mismatch, and a G/A mismatch, (iv) a G/U wobble base pair, or a U/G wobble base pair; and (v) any combination thereof. Said engineered guide RNA can be delivered via viral vector (e.g., encoded for and delivered via AAV) as disclosed herein and can be administered via any route of administration disclosed herein to a subject in need thereof. The subject may be human and may be at risk of developing or has developed alpha-1 antitrypsin deficiency. Such alpha-1 antitrypsin deficiency can be at least partially caused by a mutation of SERPINA1, for which an engineered guide RNA described herein can facilitate editing in, thus correcting the mutation in SERPINA1 and reducing the incidence of alpha-1 antitrypsin deficiency in the subject. Thus, the guide RNAs of the present disclosure can be used in a method of treatment of alpha-1 antitrypsin deficiency.

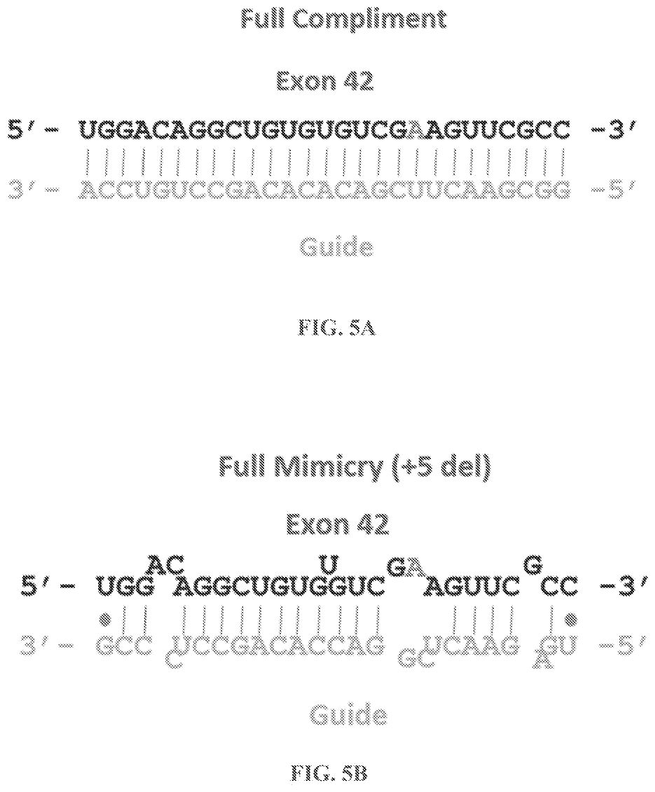

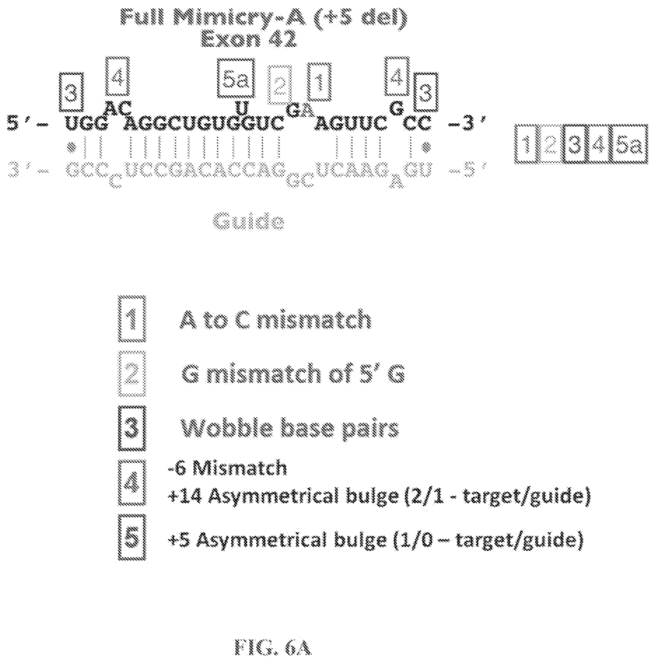

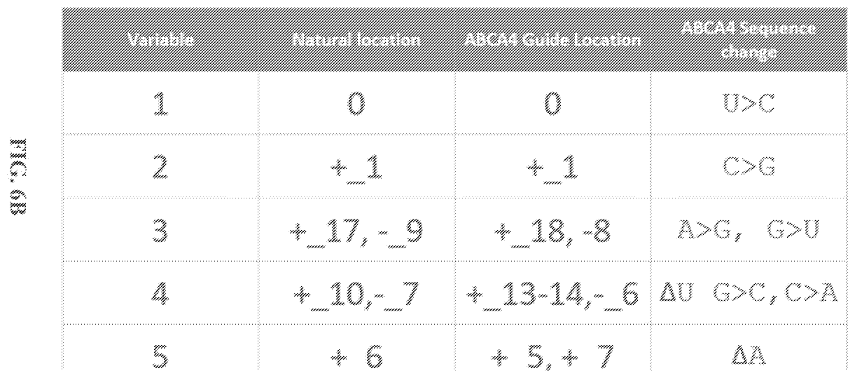

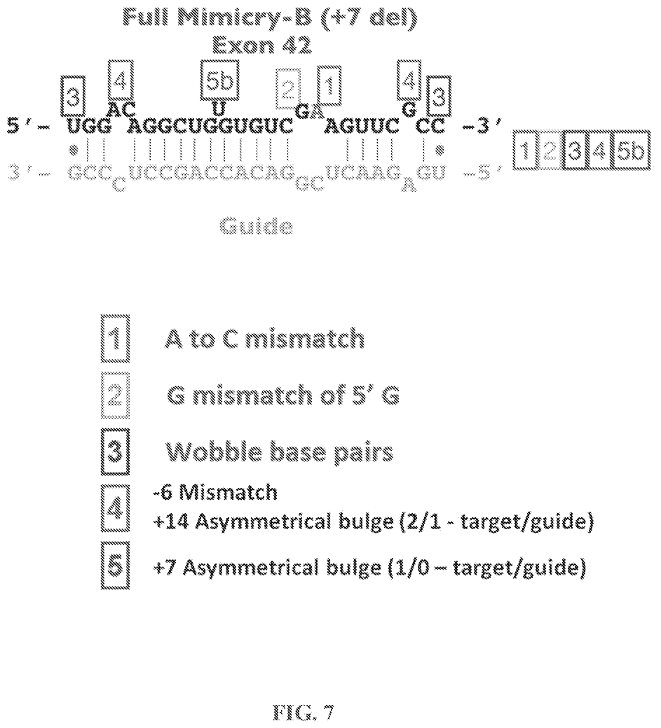

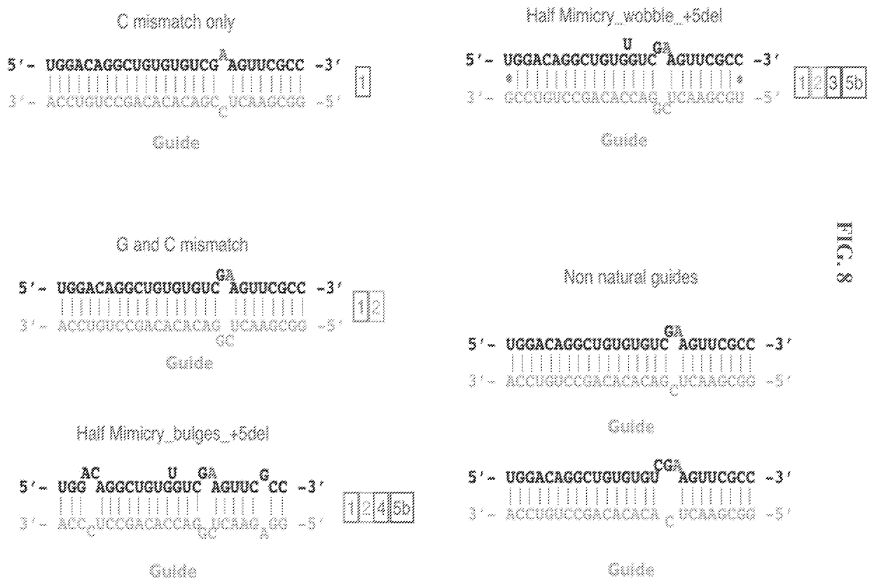

ABCA4. In some embodiments, the present disclosure provides compositions and methods of use thereof of guide RNAs that are capable of facilitating RNA editing of ATP binding cassette subfamily A member 4 (ABCA4). In some examples, the disease or condition can be associated with a mutation in an ABCA4 gene. In some examples, the disease or condition can be Stargardt macular degeneration. In some examples, the Stargardt macular degeneration can be caused, at least in part, by a mutation in an ABCA4 gene. In some examples, the mutation comprises a substitution of a G with an A at nucleotide position 5882 in a wildtype ABCA4 gene (such as accession number NC_000001.11:c94121149-93992837). In some examples, the mutation comprises a G with an A at nucleotide position 5714 in a wildtype ABCA4 gene (such as accession number NC_000001.11:c94121149-93992837). In some examples, the mutation comprises a substitution of a G with an A at nucleotide position 6320 in a wildtype ABCA4 gene (such as accession number NC_000001.11:c94121149-93992837). In some examples, the double stranded substrate mimics one or more structural features of the naturally occurring ADAR substrate and comprises a target mRNA molecule encoded by the ABCA4 gene and an engineered guide that can be complementary, at least in part, to a portion of the target mRNA molecule. FIG. 5A illustrates a double stranded substrate formed by a portion of an engineered guide described herein comprising full complimentary to a target RNA molecule encoded by an ABCA4 gene. FIG. 5B illustrates an engineered guide comprising only partial complementary to the target RNA molecule encoded by ABCA4, but adapted to form a double-stranded substrate comprising full structural mimicry (comprising all of the structural features listed and depicted in FIG. 4) of the naturally occurring ADAR substrate. For example, the double stranded substrate depicted in FIG. 5B comprises (1) an A to C mismatch, (2) a G mismatch of a 5′G, (3) two wobble base pairs, (4) a mismatch at the −6 position and an asymmetrical bulge at the +14 to +15 positions (2/1—target/guide), and (5) an asymmetrical bulge at the +5 position (1/0—target/guide), all positioned, relative to each other, similarly to the structural features comprising the naturally occurring substrate. FIGS. 6A and 6B show the full mimicry substrate (6A) compared to the naturally occurring substrate (6B), with annotations detailing each of the structural features. FIG. 6C shows a chart detailing the location of each of the structural features on the full mimicry guide and the naturally occurring substrate. FIG. 6C also details the sequence changes made to the full mimicry guide relative to a guide having full complementarity to the target sequence. FIG. 7, shows a double stranded substrate exhibiting full mimicry, with an asymmetrical bulge positioned at the +7 position relative to the target A (positioned at +7 nucleotides 5′ of the target A). Double stranded substrates with varying levels of mimicry of the naturally occurring substrate are depicted in FIG. 8. For example, as depicted in FIG. 8, the double stranded substrate can comprise an A to C mismatch only; an A to C mismatch and a G mismatch of a 5′G only; an A to C mismatch, a G mismatch of a 5′ G, and two wobble base pairs only; or an A to C mismatch, a G mismatch of a 5′ G, two wobble base pairs, and an unpaired bulge only.

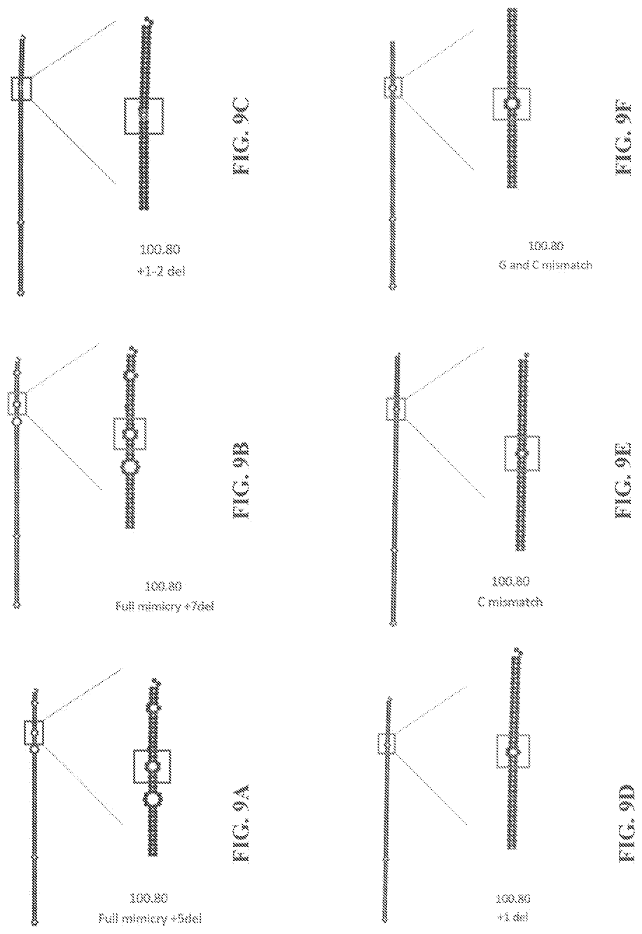

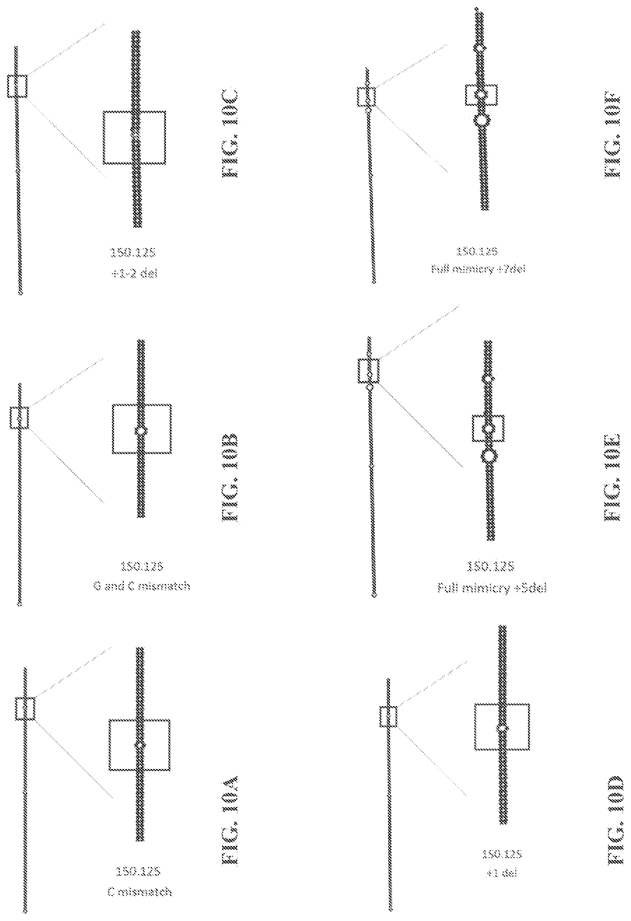

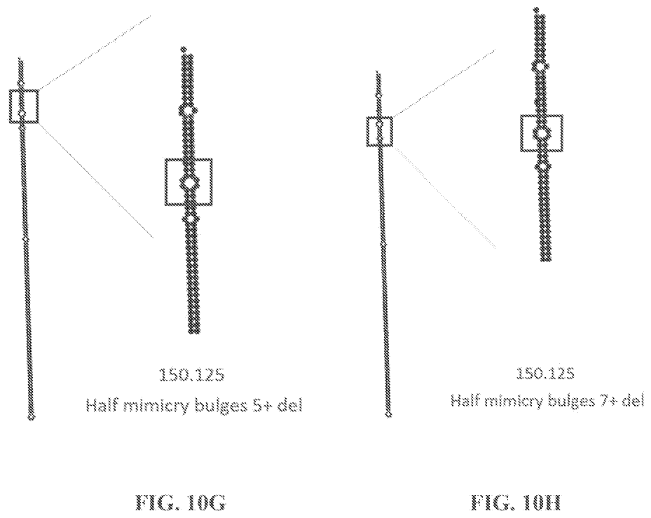

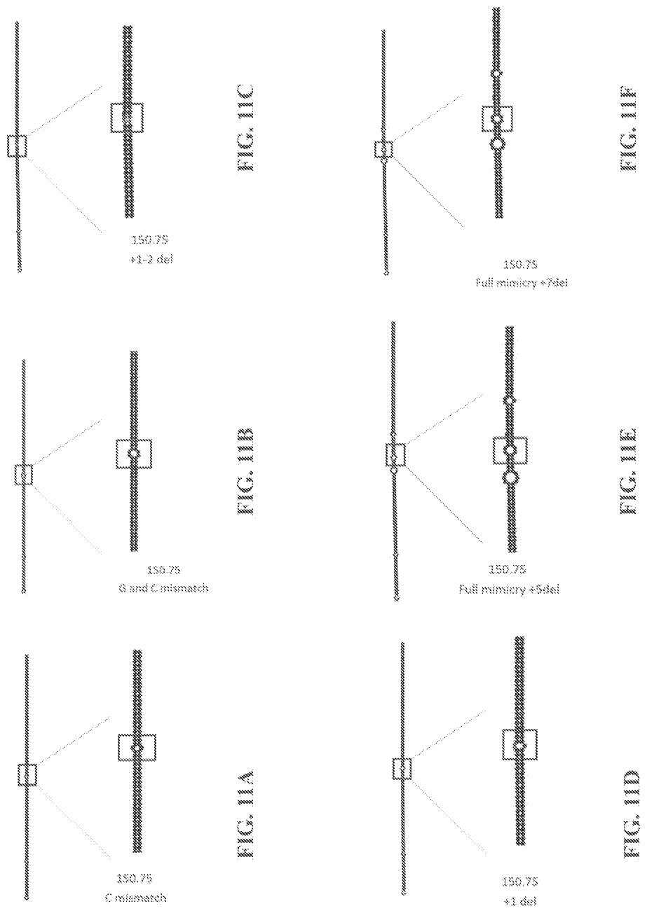

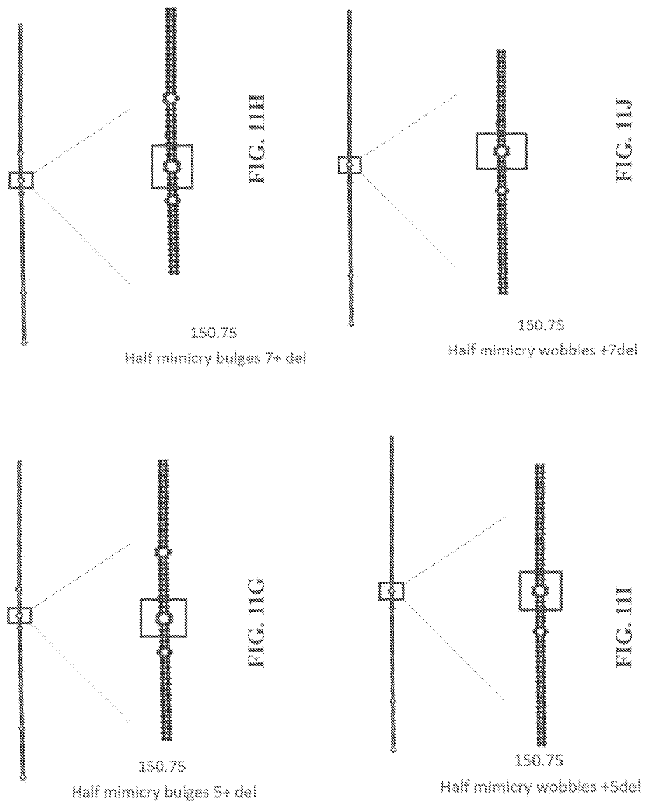

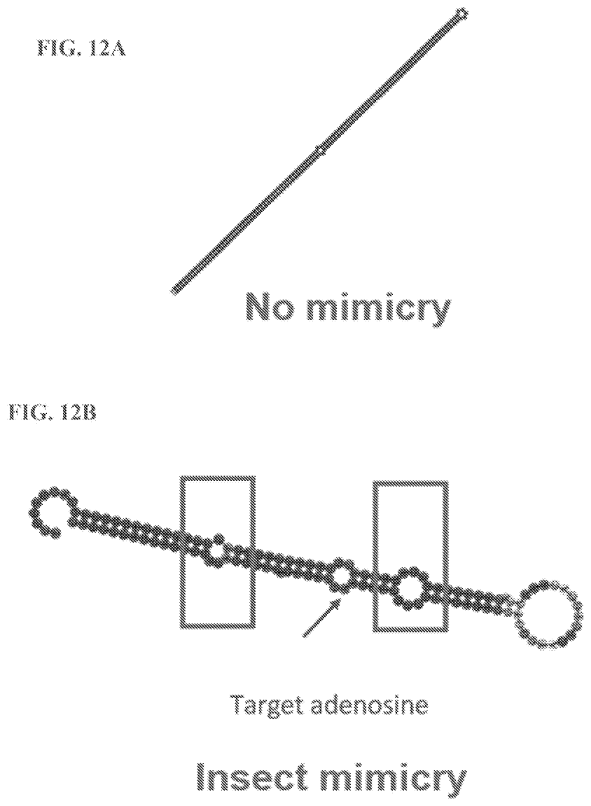

FIGS. 9-11 depict structural features of double stranded substrates formed by engineered guides described herein and target ABCA4 RNA molecules comprising varying levels of structural mimicry to the naturally occurring drosophila ADAR substrate. FIGS. 9A-9F depict substrates formed by engineered guides 100 nucleotides in length comprising, at nucleotide 80, plus or minus 2 nucleotides, from the 5′ end, a cytosine intended for pairing with the adenine to be edited by an ADAR, referred to as “100.80” guides herein. For example, “100.80” refers to a guide in which the cytosine intended for pairing with the adenine to be edited can be at nucleotide 82 from the 5′ end. FIGS. 10A-10H depict substrates formed by guides 150 nucleotides in length comprising, at nucleotide 125, plus or minus 2 nucleotides, from the 5′ end, a cytosine intended for pairing with the adenine to be edited by an ADAR, referred to as “150.125” guides herein. For example, “150.125” refers to a guide in which the cytosine intended for pairing with the adenine to be edited can be at nucleotide 123 from the 5′ end. FIGS. 11A-11J depict substrates formed by engineered guides 150 nucleotides in length comprising, at nucleotide 75, plus or minus 2 nucleotides, from the 5′ end, a cytosine intended for pairing with the adenine to be edited by an ADAR, referred to as “150.75” guides herein. For example, “150.75” refers to a guide in which the cytosine intended for pairing with the adenine to be edited can be at nucleotide 77 from the 5′ end. The guides of FIGS. 9-11 comprise a range of structural motifs mimicking that of the drosophila substrate. In some examples the engineered guide disclosed herein can be any of the guides depicted in FIGS. 9-11. Guides illustrated in FIGS. 9-11 targeting ABCA4 are presented in TABLE 9 of Example 4 of the present disclosure.

In some examples, the engineered guide targeting ABCA4 mRNA (including a latent guide RNA having latent structure) comprises a polynucleotide of any one of SEQ ID NO: 11-34, 58, 218-289, 291-296, or 328-343. In some examples, the engineered guide targeting ABCA4 mRNA (including a latent guide RNA having latent structure) comprises a polynucleotide having at least 99% identity, at least 95% identity, at least 90% identity, at least 85% identity, at least 80% identity, or at least 70% identity to any one of SEQ ID NO: 11-34, 58, 218-289, 291-296, or 328-343. In some examples, the engineered guide (including a latent guide RNA having latent structure) comprises a polynucleotide having at least 99% length, at least 95% length, at least 90% length, at least 85% length, at least 80% length, or at least 70% length to any one of SEQ ID NO: 11-34, 58, 218-289, 291-296, or 328-343. In some examples, hybridization of a latent guide RNA targeting ABCA4 to a target ABCA4 mRNA produces a guide-target RNA scaffold that comprises a structural features selected from the group consisting of: (i) one or more X₁/X₂ bulges, wherein X₁ is the number of nucleotides of the target RNA in the bulge and X₂ is the number of nucleotides of the engineered guide RNA in the bulge, and wherein the one or more bulges is a 2/1 asymmetric bulge, a 1/0 asymmetric bulge, a 2/2 symmetric bulge, a 3/3 symmetric bulge, or a 4/4 symmetric bulge; (ii) an X₁/X₂ internal loop, wherein X₁ is the number of nucleotides of the target RNA in the internal loop and X₂ is the number of nucleotides of the engineered guide RNA in the internal loop, and wherein the internal loop is a 5/5 symmetric loop (iii) one or more mismatches, wherein the one or more mismatches is a G/G mismatch, an A/C mismatch, or a G/A mismatch, (iv) a G/U wobble base pair or a U/G wobble base pair, and (v) any combination thereof. In some embodiments, the guide-target RNA scaffold comprises a 2/1 asymmetric bulge, a 1/0 asymmetric bulge, a G/G mismatch, an A/C mismatch, and a 3/3 symmetric bulge. In some instances, the engineered latent guide RNA targeting ABCA4 is the engineered latent guide RNA of SEQ ID NO: 291. In some instances, the engineered latent guide RNA targeting ABCA4 is the engineered latent guide RNA of SEQ ID NO: 291. In some instances, the engineered latent guide RNA targeting ABCA4 comprises a G/G mismatch, a U/U mismatch, and a G/G mismatch. Said engineered guide RNAs can be delivered via viral vector (e.g., encoded for and delivered via AAV) as disclosed herein and can be administered via any route of administration disclosed herein to a subject in need thereof. The subject can be human and may be at risk of developing or has developed Stargardt macular degeneration (or Stargardt's disease). Such Stargardt macular degeneration can be at least partially caused by a mutation of ABCA4, for which an engineered guide RNA described herein can facilitate editing in, thus correcting the mutation in ABCA4 and reducing the incidence of Stargardt macular degeneration in the subject. Thus, the guide RNAs of the present disclosure can be used in a method of treatment of Stargardt macular degeneration.

APP. In some embodiments, the present disclosure provides compositions and methods of use thereof of guide RNAs that are capable of facilitating RNA editing of an amyloid precursor protein (APP). In some examples, the disease or condition can be associated with expression of or cleavage products of an amyloid precursor protein (APP). In some examples, the disease or condition associated with Amyloid beta (AD or Abeta) peptide deposition in the brain or blood vessels. In some examples, the Abeta deposition can be produced by the cleavage of APP by beta secretase (BACE) or gamma secretase. In some examples, the disease can be a neurodegenerative disease. In some examples, the disease comprises Alzheimer's disease, Parkinson's disease, corticobasal degeneration, dementia with Lewy bodies, Lewy body variant of Alzheimer's disease, Parkinson's disease with dementia, Pick's disease, progressive supranuclear palsy, dementia, fronto-temporal dementia with Parkinsonism linked to tau mutations on chromosome 17, or any combination thereof. In some examples, the engineered guides (including latent guide RNAs having latent structure) can be administered to knockdown expression of APP or to edit a cut site to prevent Abeta fragment formation from APP.

Guide RNAs of the present disclosure can facilitate editing of the cleavage site in APP, so that beta/gamma secretases exhibit reduced cleavage of APP or can no longer cut APP and, therefore, reduced levels of Abeta 40/42 or no Abetas can be produced. In some embodiments, a guide RNA of the present disclosure can target any one of or any combination of the following sites in APP for RNA editing: K670E, K670R, K670G, M671V, A673V, A673T, D672G, E682G, H684R, K687R, K687E, or K687G, I712X, or T714X. Said guide RNAs targeting a site in APP can be encoded by an engineered polynucleotide construct of the present disclosure. Said engineered guide RNAs may be delivered via viral vector (e.g., encoded for and delivered via AAV) as disclosed herein and may be administered via any route of administration disclosed herein to a subject in need thereof. The subject may be human and may be at risk of developing or has developed Alzheimer's disease. The subject may be human and may be at risk of developing or has developed a neurological disease in which APP impacts disease pathology. Thus, the guide RNAs of the present disclosure having latent structure can be used in a method of treatment of neurological diseases (e.g., Alzheimer's disease).

Alpha-synuclein (SNCA). The Alpha-synuclein gene is made up of 5 exons and encodes a 140 amino-acid protein with a predicted molecular mass of ˜14.5 kDa. The encoded product is an intrinsically disordered protein with unknown functions. Usually, Alpha-synuclein is a monomer. Under certain stress conditions or other unknown causes, α-synuclein self-aggregates into oligomers. Lewy-related pathology (LRP), primarily comprised of Alpha-synuclein in more than 50% of autopsy-confirmed Alzheimer's disease patients' brains. While the molecular mechanism of how Alpha-synuclein affects the development of Alzheimer's disease is unclear, experimental evidence has shown that Alpha-synuclein interacts with Tau-p and may seed the intracellular aggregation of Tau-p. Moreover, Alpha-synuclein could regulate the activity of GSK3β, which can mediate Tau-hyperphosphorylation. Alpha-synuclein can also self-assemble into pathogenic aggregates (Lewy bodies). Both Tau and α-synuclein can be released into the extracellular space and spread to other cells. Vascular abnormalities impair the supply of nutrients and removal of metabolic byproducts, cause microinfarcts, and promote the activation of glial cells. Therefore, a multiplex strategy to substantially reduce Tau formation, alpha-synuclein formation, or a combination thereof can be important in effectively treating neurodegenerative diseases.

The domain structure of Alpha-synuclein comprises an N-terminal A2 lipid-binding alpha-helix domain, a Non-amyloid R component (NAC) domain, and a C-terminal acidic domain. The lipid-binding domain consists of five KXKEGV imperfect repeats. The NAC domain consists of a GAV motif with a VGGAVVTGV consensus sequence and three GXXX sub-motifs—where X is any of Gly, Ala, Val, Ile, Leu, Phe, Tyr, Trp, Thr, Ser or Met. The C-terminal acidic domain contains a copper-binding motif with a DPDNEA consensus sequence. Molecularly, Alpha-synuclein is suggested to play a role in neuronal transmission and DNA repair.

In some cases, a region of Alpha-synuclein can be targeted utilizing guide RNAs provided herein. In some cases, a region of the Alpha-synuclein mRNA can be targeted with the engineered guide RNAs disclosed herein for knockdown. In some cases, a region of the exon or intron of the Alpha-synuclein mRNA can be targeted. In some embodiments, a region of the non-coding sequence of the Alpha-synuclein mRNA, such as the 5′ UTR and 3′ UTR, can be targeted. In other cases, a region of the coding sequence of the Alpha-synuclein mRNA can be targeted. Suitable regions include but are not limited to a N-terminal A2 lipid-binding alpha-helix domain, a Non-amyloid R component (NAC) domain, or a C-terminal acidic domain.

In some aspects, an alpha-synuclein mRNA sequence is targeted. In some cases, any one of the 3,177 residues of the sequence may be targeted utilizing the guide RNAs provided herein. In some cases, a target residue may be located among residues 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2600, 2601-2700, 2701-2800, 2801-2900, 2901-3000, 3001-3100, and/or 3101-3177.

In some embodiments, the present disclosure provides compositions and methods of use thereof of guide RNAs that are capable of facilitating RNA editing of SNCA. In some embodiments, a guide RNA of the present disclosure can knock down expression of SNCA, for example, by facilitating editing at a 3′ UTR of an SNCA gene. Said guide RNAs targeting a site in SNCA can be encoded by an engineered polynucleotide construct of the present disclosure.

In some examples, the engineered guide targeting SNCA mRNA (including a latent guide RNA having latent structure) comprises a polynucleotide of any one of SEQ ID NO: 59-101, 104-108, and 208-217. In some examples, the engineered guide targeting SNCA mRNA (including a latent guide RNA having latent structure) comprises a polynucleotide having at least 99% identity, at least 95% identity, at least 90% identity, at least 85% identity, at least 80% identity, or at least 70% identity to any one of SEQ ID NO: 59-101, 104-108, and 208-217. In some examples, the engineered guide (including a latent guide RNA having latent structure) comprises a polynucleotide having at least 99% length, at least 95% length, at least 90% length, at least 85% length, at least 80% length, or at least 70% length to any one of SEQ ID NO: 59-101, 104-108, and 208-217. In some examples, hybridization of a latent guide RNA targeting SNCA to a target SNCA mRNA produces a guide-target RNA scaffold that comprises a structural features selected from the group consisting of: (i) an X₁/X₂ bulge, wherein X₁ is the number of nucleotides of the target RNA in the bulge and X₂ is the number of nucleotides of the engineered guide RNA in the bulge, and wherein the bulge is a 4/4 symmetric bulge; (ii) one or more X₁/X₂ internal loops, wherein X₁ is the number of nucleotides of the target RNA in the internal loop and X₂ is the number of nucleotides of the engineered guide RNA in the internal loop, and wherein the one or more internal loop is a 5/5 symmetric loop, an 8/8 symmetric loop, or a 49/4 asymmetric loop; (iii) one or more mismatches, wherein the one or more mismatches is an A/C mismatch, a G/G mismatch, a G/A mismatch, a U/C mismatch, or an A/A mismatch, (iv) any combination thereof. Said engineered guide RNA can be delivered via viral vector (e.g., encoded for and delivered via AAV) as disclosed herein and can be administered via any route of administration disclosed herein to a subject in need thereof. The subject can be human and may be at risk of developing or has developed Alzheimer's disease or Parkinson's disease. The subject can be human and may be at risk of developing or has developed a neurological disease in which overexpression of SNCA impacts disease pathology. Thus, the guide RNAs of the present disclosure can be used in a method of treatment of neurological diseases (e.g., Alzheimer's disease).

LRRK2. Leucine-rich repeat kinase 2 (LRRK2) has been associated with familial and sporadic cases of Parkinson's Disease and immune-related disorders like Crohn's disease. Its aliases include LRRK2, AURA17, DARDARIN, PARK8, RIPK7, ROCO2, or leucine-rich repeat kinase 2. The LRRK2 gene is made up of 51 exons and encodes a 2527 amino-acid protein with a predicted molecular mass of about 286 kDa. The encoded product is a multi-domain protein with kinase and GTPase activities. LRRK2 can be found in various tissues and organs including but not limited to adrenal, appendix, bone marrow, brain, colon, duodenum, endometrium, esophagus, fat, gall bladder, heart, kidney, liver, lung, lymph node, ovary, pancreas, placenta, prostate, salivary gland, skin, small intestine, spleen, stomach, testis, thyroid, and urinary bladder. LRRK2 can be ubiquitously expressed but is generally more abundant in the brain, kidney, and lung tissue. Cellularly, LRRK2 has been found in astrocytes, endothelial cells, microglia, neurons, and peripheral immune cells.

Over 100 mutations have been identified in LRRK2; six of them-G2019S, R1441C/G/H, Y1699C, and I2020T—have been shown to cause Parkinson's Disease through segregation analysis. G2019S and R1441C are the most common disease-causing mutations in inherited cases. In sporadic cases, these mutations have shown age-dependent penetrance: The percentage of individuals carrying the G2019S mutation that develops the disease jumps from 17% to 85% when the age increases from 50 to 70 years old. In some cases, mutation-carrying individuals never develop the disease.

At its catalytic core, LRRK2 contains the Ras of complex proteins (Roc), C-terminal of ROC (COR), and kinase domains. Multiple protein-protein interaction domains flank this core: an armadillo repeats (ARM) region, an ankyrin repeat (ANK) region, a leucine-rich repeat (LRR) domain are found in the N-terminus joined by a C-terminal WD40 domain. The G2019S mutation is located within the kinase domain. It has been shown to increase the kinase activity; for R1441C/G/H and Y1699C, these mutations can decrease the GTPase activity of the Roc domain. Genome-wide association study has found that common variations in LRRK2 increase the risk of developing sporadic Parkinson's Disease. While some of these variations are nonconservative mutations that affect the protein's binding or catalytic activities, others modulate its expression. These results suggest that specific alleles or haplotypes can regulate LRRK2 expression.

Pro-inflammatory signals upregulate LRRK2 expression in various immune cell types, suggesting that LRRK2 is a critical regulator in the immune response. Studies have found that both systemic and central nervous system (CNS) inflammation are involved in Parkinson's Disease's symptoms. Moreover, LRRK2 mutations associated with Parkinson's Disease modulate its expression levels in response to inflammatory stimuli. Many mutations in LRRK2 are associated with immune-related disorders such as inflammatory bowel disease such as Crohn's Disease. For example, both G2019S and N2081D increase LRRK2's kinase activity and are over-represented in Crohn's Disease patients in specific populations. Because of its critical role in these disorders, LRRK2 is an important therapeutic target for Parkinson's Disease and Crohn's Disease. In particular, many mutations, such as point mutations including G2019S, play roles in developing these diseases, making LRRK2 an attractive for therapeutic strategy such as RNA editing.

In some embodiments, the present disclosure provides compositions and methods of use thereof of guide RNAs that are capable of facilitating RNA editing of LRRK2. In some embodiments, a guide RNA of the present disclosure can target the following mutations in LRRK2: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, 51647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, or Q2490NfsX3. Said guide RNAs targeting a site in LRRK2 can be encoded by an engineered polynucleotide construct of the present disclosure.

In some examples, the engineered guide targeting LRRK2 mRNA (including a latent guide RNA having latent structure) comprises a polynucleotide of any one of SEQ ID NO: 35-42, 46-52, 111-207, or 344-345. In some examples, the engineered guide targeting LRRK2 mRNA (including a latent guide RNA having latent structure) comprises a polynucleotide having at least 99% identity, at least 95% identity, at least 90% identity, at least 85% identity, at least 80% identity, or at least 70% identity to any one of SEQ ID NO: 35-42, 46-52, 111-207, or 344-345. In some examples, the engineered guide (including a latent guide RNA having latent structure) comprises a polynucleotide having at least 99% length, at least 95% length, at least 90% length, at least 85% length, at least 80% length, or at least 70% length to any one of SEQ ID NO: 35-42, 46-52, 111-207, or 344-345. In some examples, hybridization of a latent guide RNA targeting LRRK2 to a target LRRK2 mRNA produces a guide-target RNA scaffold that comprises a structural features selected from the group consisting of: (i) one or more X₁/X₂ bulges, wherein X₁ is the number of nucleotides of the target RNA in the bulge and X₂ is the number of nucleotides of the engineered guide RNA in the bulge, and wherein the one or more bulges is a 0/1 asymmetric bulge, a 2/2 symmetric bulge, a 3/3 symmetric bulge, or a 4/4 symmetric bulge; (ii) one or more X₁/X₂ internal loops, wherein X₁ is the number of nucleotides of the target RNA in the internal loop and X₂ is the number of nucleotides of the engineered guide RNA in the internal loop, and wherein the one or more internal loops is a 5/0 asymmetric internal loop, a 5/4 asymmetric internal loop, a 5/5 symmetric internal loop, a 6/6 symmetric internal loop, a 7/7 symmetric internal loop, or a 10/10 symmetric internal loop; (iii) one or more mismatches, wherein the one or more mismatches is an A/C mismatch, an A/G mismatch, a C/U mismatch, a G/A mismatch, or a C/C mismatch, (iv) a G/U wobble base pair or a U/G wobble base pair, and (v) any combination thereof. Said engineered guide RNAs can be delivered via viral vector (e.g., encoded for and delivered via AAV) as disclosed herein and can be administered via any route of administration disclosed herein to a subject in need thereof. The subject can be human and may be at risk of developing or has developed a disease or condition associated with mutations in LRRK2 (e.g. diseases of the central nervous system (CNS) or gastrointestinal (GI) tract). For example, such diseases of conditions can include Crohn's disease or Parkinson's disease. Such CNS or GI tract diseases (e.g. Crohn's disease or Parkinson's disease) can be at least partially caused by a mutation of LRRK2, for which an engineered guide RNA described herein can facilitate editing in, thus correcting the mutation in LRRK2 and reducing the incidence of the CNS or GI tract disease in the subject. Thus, the guide RNAs of the present disclosure can be used in a method of treatment of diseases such as Crohn's disease or Parkinson's disease.

An engineered guide RNA of the present disclosure containing latent structures can have increased on-target editing via an RNA editing entity, relative to an otherwise comparable guide RNA lacking latent structures. In some embodiments, an engineered guide RNA of the present disclosure has at least about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, 50-fold, 51-fold, 52-fold, 53-fold, 54-fold, 55-fold, 56-fold, 57-fold, 58-fold, 59-fold, 60-fold, 61-fold, 62-fold, 63-fold, 64-fold, 65-fold, 66-fold, 67-fold, 68-fold, 69-fold, 70-fold, 71-fold, 72-fold, 73-fold, 74-fold, 75-fold, 76-fold, 77-fold, 78-fold, 79-fold, 80-fold, 81-fold, 82-fold, 83-fold, 84-fold, 85-fold, 86-fold, 87-fold, 88-fold, 89-fold, 90-fold, 91-fold, 92-fold, 93-fold, 94-fold, 95-fold, 96-fold, 97-fold, 98-fold, 99-fold, or 100-fold improvement in on-target editing via an RNA editing entity, relative to the otherwise comparable guide RNA lacking the latent structures. In some instances, an engineered guide RNA of the present disclosure containing latent structures has an on-target editing of at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99% for ADAR1. In some instances, an engineered guide RNA of the present disclosure containing latent structures has an on-target editing of at least about 5%, 6%, 7%, 8%, 9%, 10%, 11%, 12%, 13%, 14%, 1%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or greater than 99% for ADAR2. In some instances, an engineered guide RNA of the present disclosure containing latent structures has an on-target specificity for ADAR1 of at least about 1.00, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or greater than 2.00. In some instances, an engineered guide RNA of the present disclosure containing latent structures has an on-target specificity for ADAR2 of at least about 1.00, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or greater than 2.00.

Indications

An engineered guide RNA or engineered polynucleotide encoding the same can be administered to a subject to treat a disease or condition described herein. In some cases, a disease or condition comprises a neurodegenerative disease, a muscular disorder, a metabolic disorder, an ocular disorder (e.g. an ocular disease), a cancer, a liver disease (e.g., Alpha-1 antitrypsin (AAT) deficiency), or any combination thereof. In some examples, the disease comprises cystic fibrosis, albinism, alpha-1-antitrypsin deficiency, Alzheimer disease, Amyotrophic lateral sclerosis, Asthma, 0-thalassemia, Cadasil syndrome, Charcot-Marie-Tooth disease, Chronic Obstructive Pulmonary Disease (COPD), dementia, Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Parkinson's disease, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, Wolman disease, X-linked immunodeficiency, various forms of cancer (e.g., BRCA1 and 2 linked breast cancer and ovarian cancer). In some cases, a treatment of a disease or condition such as a neurodegenerative disease (e.g. Alzheimer's, Parkinson's) can comprise producing an edit, a knockdown or both of amyloid precursor protein (APP), tau, alpha-synuclein, or any combination thereof. In some cases, APP, tau, and alpha-synuclein can comprise a pathogenic variant. In some instances, APP can comprise a pathogenic variant such as A673V mutation or A673T mutation. In some cases, a treatment of a disease or condition such as a neurodegenerative disease (Parkinson's) can comprise producing an edit, a knockdown or both of a pathogenic variant of LRRK2. In some cases, a pathogenic variant of LRRK can comprise a G2019S mutation. The disease or condition can comprise a muscular dystrophy, an omithine transcarbamylase deficiency, a retinitis pigmentosa, a breast cancer, an ovarian cancer, Alzheimer's disease, pain, Stargardt macular dystrophy, Charcot-Marie-Tooth disease, Rett syndrome, or any combination thereof.

In some examples, the disease or condition can be caused or contributed to, at least in part, by a protein encoded by an mRNA comprising a premature stop codon. In some cases, the premature stop codon results in a truncated version of the polypeptide or protein. In some cases, the disease, disorder, or condition can be caused by an increased level of a truncated version of the polypeptide, or a decreased level of substantially full-length polypeptide. In some examples, the premature stop codon can be created by a point mutation. In some examples, the premature stop codon can be produced by a point mutation on an mRNA molecule in combination with two additional nucleotides. In some examples, the mRNA molecule comprises one, two, three, or for premature stop codons. In some examples, the disease or condition can be caused or contributed to, at least in part, by a splice site mutation on a pre-mRNA molecule. In some examples, the splice site mutation facilitates unintended splicing of a pre-mRNA molecule. In some examples, the splice site mutation results in mistranslation and/or truncation of a protein caused by incorrect delineation of a pre-mRNA splice site.

In some examples, in methods disclosed herein, the subject can be diagnosed with the disease or condition. In some examples, the subject can be diagnosed with the disease or condition by an in vitro assay.

In some examples, administration of a composition or engineered guide disclosed herein: (a) decreases expression of a gene relative to an expression of the gene prior to administration; (b) edits at least one point mutation in a subject, such as a subject in need thereof; (c) edits at least one stop codon in the subject to produce a readthrough of a stop codon; (d) produces an exon skip in the subject, or (e) any combination thereof.

### Administration and Additional Therapies

Methods described herein can comprise administration to a subject one or more engineered guide RNAs, engineered polynucleotides encoding the same, as well as compositions, pharmaceutical compositions, vectors, cells and isolated cells containing the same as described herein. Methods of determining the most effective means and dosage of administration can vary with the composition used for therapy, the purpose of the therapy, the target cell being treated, and the subject being treated.

In some examples, administration of the engineered guide RNA, engineered polynucleotide, composition, pharmaceutical composition, vector, or cell disclosed herein can be performed for a treatment duration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some examples, administration of the engineered guide RNA, engineered polynucleotide, composition, pharmaceutical composition, vector, or cell disclosed herein can be performed for a treatment duration of no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days.

In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

In some examples, administration of the engineered guide RNA, engineered polynucleotide, composition, pharmaceutical composition, vector, or cell disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. In some examples, administration can be performed repeatedly over a lifetime of a subject, such as once a month or once a year for the lifetime of a subject. In some examples, administration can be performed repeatedly over a substantial portion of a subject's life, such as once a month or once a year for at least about 1 year, 5 years, 10 years, 15 years, 20 years, 25 years, 30 years, or more.

In some examples, administration of the engineered guide RNA, engineered polynucleotide, composition, pharmaceutical composition, vector, or cell disclosed herein can be performed at least 1, 2, 3, 4, 5, 6, 7, 8, 9, 10, 11, 12, 13, 14, 15, 16, 17, 18, 19, 20, 21, 22, 23, or 24 times a day. In some examples, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some examples, administration of an engineered guide RNA disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some examples, an engineered guide RNA, engineered polynucleotide, composition, pharmaceutical composition, vector, or cell disclosed herein can be administered/applied as a single dose or as divided doses. In some examples, engineered guides RNA disclosed herein can be administered at a first time point and a second time point. In some examples, an engineered guide RNA disclosed herein can be administered such that a first administration can be administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

A method of administration can be by inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavemous, intracavitary, intracerebroventricular, intracistemal, intracomeal, intracoronal, intracoronary, intracorpous cavemaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration. Delivery can include topical administration (such as a lotion, a cream, an ointment) to an external surface of a surface, such as a skin. In some cases, administration is by parenchymal injection, intra-thecal injection, intra-ventricular injection, intra-cisternal injection, intravenous injection, or intranasal administration or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). A medical professional can administer the composition. In some cases, a cosmetic professional can administer the composition.

In some examples, a pharmaceutical composition disclosed herein can be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

In some examples, methods described herein can comprise administering a co-therapy. In some examples. a co-therapy can comprise a cancer treatment (e.g. radiotherapy, chemotherapy, CAR-T therapy, immunotherapy, hormony therapy, cryoablation). In some examples, a co-therapy can comprise surgery. In some example, a co-therapy can comprise a laser therapy.

In some examples, the pharmaceutical composition comprises a first active ingredient (e.g., an engineered guide RNA disclosed herein, a composition disclosed herein, an isolated cell disclosed herein, or an isolated plurality of cells disclosed herein). In some examples, the pharmaceutical can comprise a second, third or fourth active ingredient. In some examples, the pharmaceutical composition comprises an additional therapeutic agent. In some examples, the second, third, or fourth active ingredient can be the additional therapeutic agent. In some examples, the additional therapeutic agent treats macular degeneration. In some examples, the additional therapeutic agent can be for treating a neurological disease or disorder (e.g., Parkinson's disease, Alzheimer's disease, or dementia). In some examples, the additional therapeutic agent can be for treating a liver disease or disorder (e.g., liver cirrhosis or alpha-1 antitrypsin deficiency). In some instances, amyloid-beta aggregation (Abeta plaques) contribute to the pathology of Alzheimer's disease. Abeta can be derived from sequential proteolysis of amyloid precursor protein (APP) as variable-length fragments. In some examples, the additional therapeutic agent can be for preventing beta-amyloid from clumping into plaques or remove beta-amyloid plaques that have formed.

In some examples, the additional therapeutic agent can be a 5-HT 6 antagonist, a 5-HT2A inverse agonist, an AB42 lowering agent, an acetylcholinesterase inhibitor, an alpha secretase enhancer, an alpha-1 adrenoreceptor antagonist, an ammonia reducer, an angiotensin II receptor blocker, an alpha-2 adrenergic agonist, an anti-amyloid antibody, an anti-aggregation agent, an anti-amyloid immunotherapy, an anti-inflammatory agent, a glial cell modulator, an antioxidant, anti-tau antibody, an anti-tau immunotherapy, an anti-VEGF agent, an antiviral drug, a BACE inhibitor, a beta-adrenergic blocking agents, a beta-2 andrenergic receptor agonist, an arginase inhibitor, a beta blocker, a beta-HSD1 inhibitor, a calcium channel blocker, a cannabinoid, a CBT or CB2 endocannabinoid receptor agonist, a cholesterol lowering agent, a D2 receptor agonist, a dopamine-norepinephrine reuptake inhibitor, a FLNA inhibitor, a gamma secretase inhibitor, a GABA receptor modulator, a glucagon-like peptide 1 receptor agonist, a glutamate modulator, a glutamate receptor antagonist, a glycine transporter 1 inhibitor, a gonadotropin-releasing hormone receptor agonist, a GSK-3B inhibitor, a hepatocyte growth factor, a histone deacetylase inhibitor, a IgG1-Fc-GAIM fusion protein, an ion channel modulator, an iron chelating agent, a meukotriene receptor antagonist, a MAPT RNA inhibitor, a mast cell stabilizer, a melatonin receptor agonist, a microtubule protein modulator, a mitochondrial ATP synthase inhibitor, a monoamine oxidase B inhibitor, a muscarinic agonist, a nicotinic acetylcholine receptor agonist, an NMDA antagonist, an NMDA receptor modulator, a nonhormonal estrogen receptor B agonist, a nonnucleoside reverse transcriptase inhibitor, a nonsteroidal anti-inflammatory agent, an omega-3 fatty acid, a P38 MAPK inhibitor, a P75 neurotrophin receptor ligand, a PDE 5 inhibitor, a PDE-3 inhibitor, a PDE4D inhibitor, a positive allosteric modulator of GABA-A receptors, a PPAR-gamma agonist, a protein kinase C modulator, a RIPK1 inhibitor, a secretase inhibitor, a selective inhibitor of APP production, a selective norepinephrine reuptake inhibitor, a selective serotonin reuptake inhibitor, a selective tyrosine kinase inhibitor, a SGLT2 inhibitor, a SIGLEC-3 inhibitor, a sigma-1 receptor agonist, a sigma-2 receptor antagonist, a stem cell therapy, an SV2A modulator, a synthetic hormone, a synthetic granulocyte colony stimulator, synthetic thiamine, a tau protein aggregation inhibitor, a telomerase reverse transcriptase vaccine, a thrombin inhibitor, a transport protein ABCC1 activator, a TREM2 inhibitor, a vascular endothelial growth factor (VEGF) inhibitor, a vitamin or any combination thereof.

In some examples, the additional therapeutic agent can be an ammonia reducer, a beta blocker, a synthetic hormone, an antibiotic, or an antiviral drug, a vascular endothelial growth factor (VEGF) inhibitor, a stem cell treatment, a vitamin or modified form thereof, or any combination thereof.

In some examples, the additional therapeutic agent can be AADvac1, AAVrh.10hAPOE2, ABBV-8E12, ABvac40, AD-35, aducanumab, aflibercept, AGB101, AL002, AL003, allopregnanolone, amlopidine, AMX0035, ANAVEX 2-73, APH-1105, AR1001, AstroStem, atorvastatin, AVP-786, AXS-05, BAC, benfotiamine, BHV4157, BI425809, BIIB092, BIIP06, bioactive dietary polyphenol preparation, BPN14770, brexpiprazole, brolucizumab, byrostatin, CAD106, candesartan, CERE-110, cilostazol, CKD-355, CNP520, COR388, crenezumab, cromolyn, CT1812, curcumin, dabigatran, DAOI, dapagliflozin, deferiprone, DHA, DHP1401, DNL747, dronabinol, efavirenz, elderberry juice, elenbecestat, escitalopram, formoterol, gantenerumab, Ginkgo biloba, grapeseed extract, GRF6019, guanfacine, GV1001, hUCB-MSCs, ibuprofen, icosapent ethyl, ID1201, insulin aspart, insulin glulisine, IONIS MAPTRx, J147, JNJ-63733657, lactulose, lactitol, lemborexant, leuprolide acetate depot, levetiracetam, liraglutide, lithium, LM11A-31-BHS, losartan, L-serine, L-ornithine phenylacetate, Lu AF20513, LY3002813, LY3303560, LY3372993, masitinib, methylene blue, methylphenidate, metronidazole, mirtazapine, ML-4334, MLC901, montelukast, MP-101, nabilone, NDX-1017, neflamapimod, neomycin, nicotinamide, nicotine, nilotinib, nitazoxanide, NPT08, octagam 10%, octohydroaminoacridine succinate, omega-3 PUFA, perindopril, pimavanserin, piromelatine, posiphen, prazosin, PTI-125, ranibizumab rasagiline, rifaximin, riluzole, R07105705, RPh201, sagramostim, salsalate, S-equol, sodium benzoate, sodium phenylacetate, solanezumab, SUVN-502, telmisartan, TEP, THN201, TPI-287, traneurocin, TRx0237, UB-311, valacyclovir, venlafaxine hMSCs (human mesenchymal stem cells), vorinostat, xanamem, zolpidem, or any combination thereof.

### Compositions

### Vectors

In some examples, the delivery vehicle comprises a delivery vector. In some examples, the delivery vector comprises DNA, such as double stranded or single stranded DNA. In some examples, the vector comprises RNA. In some examples, the delivery vehicle comprises one or more delivery vectors. In some examples, the one or more delivery vectors comprise an engineered guide disclosed herein. In some examples, the one or more delivery vectors comprises a polynucleotide encoding an engineered guide disclosed herein. In some examples, one delivery vector comprises a polynucleotide encoding an engineered guide RNA disclosed herein. In some examples, one delivery vector comprises a polynucleotide encoding a portion of an engineered guide RNA disclosed herein and a second delivery vector encodes a portion of an engineered guide RNA disclosed herein.

In some examples, the delivery vector can be a eukaryotic vector, a prokaryotic vector (e.g., a bacterial vector) a viral vector, or any combination thereof. In some examples, the delivery vector can be a viral vector. In some examples, the viral vector can be a retroviral vector, an adenoviral vector, an adeno-associated viral vector, an alphavirus vector, a lentivirus vector (e.g., human or porcine), a Herpes virus vector, an Epstein-Barr virus vector, an SV40 virus vectors, a pox virus vector, or a combination thereof. In some examples, the viral vector can be a recombinant vector, a hybrid vector, a chimeric vector, a self-complementary vector, a single-stranded vector or any combination thereof.

In some examples, the viral vector can be as adeno-associated virus (AAV). In some examples, the viral vector can be of a specific serotype. In some examples, the viral vector can be an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, AAV 12 serotype, AAV13 serotype, AAV 14 serotype, AAV 15 serotype, AAV 16 serotype, AAV.rh8 serotype, AAV.rh10 serotype, AAV.rh20 serotype, AAV.rh39 serotype, AAV.Rh74 serotype, AAV.RHM4-1 serotype, AAV.hu37 serotype, AAV.Anc80 serotype, AAV.Anc80L65 serotype, AAV.7m8 serotype, AAV.PHP.B serotype, AAV2.5 serotype, AAV2tYF serotype, AAV3B serotype, AAV.LK03 serotype, AAV.HSC1 serotype, AAV.HSC2 serotype, AAV.HSC3 serotype, AAV.HSC4 serotype, AAV.HSC5 serotype, AAV.HSC6 serotype, AAV.HSC7 serotype, AAV.HSC8 serotype, AAV.HSC9 serotype, AAV.HSC10 serotype, AAV.HSC11 serotype, AAV.HSC12 serotype, AAV.HSC13 serotype, AAV.HSC14 serotype, AAV.HSC15 serotype, AAV.HSC16 serotype or AAVhu68 serotype a derivative of any of these, or any combination thereof.

In some examples, the AAV vector can be a recombinant vector, a hybrid AAV vector, a chimeric AAV vector, a self-complementary AAV (scAAV) vector, a single-stranded AAV or any combination thereof.

In some examples, the AAV vector can be a recombinant AAV (rAAV) vector. Methods of producing recombinant AAV vectors generally involve, in some cases, introducing into a producer cell line: (1) DNA necessary for AAV replication and synthesis of an AAV capsid, (b) one or more helper constructs comprising the viral functions missing from the AAV vector (c) a helper virus, and (d) the plasmid construct containing the genome of the AAV vector, e.g., ITRs, promoter and transgene (e.g., an engineered guide disclosed herein) sequences, etc. In some examples, the viral vectors described herein can be engineered through synthetic or other suitable means by references to published sequences, such as can be available in the literature. For example, the genomic and protein sequences of various serotypes of AAV, as well as the sequences of the native terminal repeats (TRs), Rep proteins, and capsid subunits can be found in the literature or in public databases such as GenBank or Protein Data Bank (PDB).

In some examples, methods of producing delivery vectors herein comprising packaging an engineered guide disclosed herein in an AAV vector. In some examples, methods of producing the delivery vectors described herein comprise, (a) introducing into a cell: (i) a polynucleotide encoding any engineered guide RNA disclosed herein; and (ii) a viral genome comprising a Replication (Rep) gene and Capsid (Cap) gene that encodes a wild-type AAV capsid protein or modified version thereof; (b) expressing in the cell the wild-type AAV capsid protein or modified version thereof; (c) assembling an AAV particle; and (d) packaging the polynucleotide encoding the engineered guide RNA in the AAV particle, thereby generating an AAV delivery vector. In some examples, any engineered guide RNA disclosed herein, promoters, stuffer sequences, and any combination thereof can be packaged in the AAV vector. In some examples, the AAV vector can package 1, 2, 3, 4, or 5 copies of the engineered guide RNA. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site.

In some examples, a hybrid AAV vector can be produced by transcapsidation, e.g., packaging an inverted terminal repeat (ITR) from a first serotype into a capsid of a second serotype, wherein the first and second serotypes can be not the same. In some examples, the Rep gene and ITR from a first AAV serotype (e.g., AAV2) can be used in a capsid from a second AAV serotype (e.g., AAV9), wherein the first and second AAV serotypes may not be the same. As a non-limiting example, a hybrid AAV serotype comprising the AAV2 ITRs and AAV9 capsid protein can be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector can be a chimeric AAV vector. In some examples, the chimeric AAV vector comprises an exogenous amino acid or an amino acid substitution, or capsid proteins from two or more serotypes. In some examples, a chimeric AAV vector can be genetically engineered to increase transduction efficiency, selectivity, or a combination thereof.

In some examples, the AAV vector comprises a self-complementary AAV genome. Self-complementary AAV genomes can contain both DNA strands which can anneal together to form double-stranded DNA.

In some examples, the delivery vector can be a retroviral vector. In some examples, the retroviral vector can be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector can be transfected such that the majority of sequences coding for the structural genes of the virus (e.g., gag, pol, and env) can be deleted and replaced by the gene(s) of interest.

In some examples, the delivery vehicle can be a non-viral vector. In some examples, the delivery vehicle can be a plasmid. In some embodiments, the plasmid comprises DNA. In some embodiments, the plasmid comprises RNA. In some examples, the plasmid comprises circular double-stranded DNA. In some examples, the plasmid can be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid can be a minicircle plasmid. In some examples, the plasmid contains one or more genes that provide a selective marker to induce a target cell to retain the plasmid. In some examples, the plasmid can be formulated for delivery through injection by a needle carrying syringe. In some examples, the plasmid can be formulated for delivery via electroporation. In some examples, the plasmids can be engineered through synthetic or other suitable means known in the art. For example, in some cases, the genetic elements can be assembled by restriction digest of the desired genetic sequence from a donor plasmid or organism to produce ends of the DNA which can then be readily ligated to another genetic sequence.

In some examples, an isolated cell or cells comprise any of the engineered guides or delivery vectors disclosed herein. In some examples, the isolated cell or cells comprise one or more human cells. In some examples, the isolated cell or cells comprise one or more T-Cells. In some examples, the isolated cell or cells comprise one or more HEK293 cells.

### Pharmaceutical Compositions

Disclosed herein, in certain embodiments, are pharmaceutical compositions comprising an engineered guide RNA disclosed herein, a composition disclosed herein, an isolated cell disclosed herein, or an isolated plurality of cells disclosed herein and a pharmaceutically acceptable excipient, carrier or diluent. In some examples, the pharmaceutical composition comprises an engineered guide RNA disclosed herein and a pharmaceutically acceptable excipient, carrier or diluent. In some examples, the pharmaceutical composition comprises an engineered polynucleotide encoding an engineered guide RNA disclosed herein and a pharmaceutically acceptable excipient, carrier or diluent. In some examples, the pharmaceutical composition comprises a delivery vector disclosed herein and a pharmaceutically acceptable excipient, carrier or diluent. In some examples, the pharmaceutical composition comprises an isolated cell (e.g. comprising a delivery vector disclosed herein) or plurality of cells disclosed herein and a pharmaceutically acceptable excipient, carrier or diluent.

In some examples, the pharmaceutical composition comprises a first active ingredient (e.g., an engineered guide disclosed herein, a composition disclosed herein, an isolated cell disclosed herein, or an isolated plurality of cells disclosed herein). In some examples, the pharmaceutical can comprise a second, third or fourth active ingredient. In some examples, the pharmaceutical composition comprises an additional therapeutic agent. In some examples, the second, third, or fourth active ingredient can be the additional therapeutic agent. In some examples, the additional therapeutic agent treats macular degeneration. In some examples, the additional therapeutic agent comprises. In some examples, the additional therapeutic agent can be for treating a neurological disease or disorder (e.g., Parkinson's disease, Alzheimer's disease, or dementia). In some examples, the additional therapeutic agent can be for treating a liver disease or disorder (e.g., liver cirrhosis or alpha-1 antitrypsin deficiency). In some instances, amyloid-beta aggregation (Abeta plaques) contribute to the pathology of Alzheimer's disease. Abeta can be derived from sequential proteolysis of amyloid precursor protein (APP) as variable-length fragments. In some examples, the additional therapeutic agent can be for preventing beta-amyloid from clumping into plaques or remove beta-amyloid plaques that have formed.

In some examples, the additional therapeutic agent can be a 5-HT 6 antagonist, a 5-HT2A inverse agonist, an AB42 lowering agent, an acetylcholinesterase inhibitor, an alpha secretase enhancer, an alpha-1 adrenoreceptor antagonist, an ammonia reducer, an angiotensin II receptor blocker, an alpha-2 adrenergic agonist, an anti-amyloid antibody, an anti-aggregation agent, an anti-amyloid immunotherapy, an anti-inflammatory agent, a glial cell modulator, an antioxidant, anti-tau antibody, an anti-tau immunotherapy, an anti-VEGF agent, an antiviral drug, a BACE inhibitor, a beta-adrenergic blocking agents, a beta-2 andrenergic receptor agonist, an arginase inhibitor, a beta blocker, a beta-HSD1 inhibitor, a calcium channel blocker, a cannabinoid, a CB1 or CB2 endocannabinoid receptor agonist, a cholesterol lowering agent, a D2 receptor agonist, a dopamine-norepinephrine reuptake inhibitor, a FLNA inhibitor, a gamma secretase inhibitor, a GABA receptor modulator, a glucagon-like peptide 1 receptor agonist, a glutamate modulator, a glutamate receptor antagonist, a glycine transporter 1 inhibitor, a gonadotropin-releasing hormone receptor agonist, a GSK-3B inhibitor, a hepatocyte growth factor, a histone deacetylase inhibitor, a IgG1-Fc-GAIM fusion protein, an ion channel modulator, an iron chelating agent, a meukotriene receptor antagonist, a MAPT RNA inhibitor, a mast cell stabilizer, a melatonin receptor agonist, a microtubule protein modulator, a mitochondrial ATP synthase inhibitor, a monoamine oxidase B inhibitor, a muscarinic agonist, a nicotinic acetylcholine receptor agonist, an NMDA antagonist, an NMDA receptor modulator, a nonhormonal estrogen receptor B agonist, a nonnucleoside reverse transcriptase inhibitor, a nonsteroidal anti-inflammatory agent, an omega-3 fatty acid, a P38 MAPK inhibitor, a P75 neurotrophin receptor ligand, a PDE 5 inhibitor, a PDE-3 inhibitor, a PDE4D inhibitor, a positive allosteric modulator of GABA-A receptors, a PPAR-gamma agonist, a protein kinase C modulator, a RIPK1 inhibitor, a secretase inhibitor, a selective inhibitor of APP production, a selective norepinephrine reuptake inhibitor, a selective serotonin reuptake inhibitor, a selective tyrosine kinase inhibitor, a SGLT2 inhibitor, a SIGLEC-3 inhibitor, a sigma-1 receptor agonist, a sigma-2 receptor antagonist, a stem cell therapy, an SV2A modulator, a synthetic hormone, a synthetic granulocyte colony stimulator, synthetic thiamine, a tau protein aggregation inhibitor, a telomerase reverse transcriptase vaccine, a thrombin inhibitor, a transport protein ABCC1 activator, a TREM2 inhibitor, a vascular endothelial growth factor (VEGF) inhibitor, a vitamin or any combination thereof.

In some examples, the additional therapeutic agent can be an ammonia reducer, a beta blocker, a synthetic hormone, an antibiotic, or an antiviral drug, a vascular endothelial growth factor (VEGF) inhibitor, a stem cell treatment, a vitamin or modified form thereof, or any combination thereof.

In some examples, the additional therapeutic agent can be AADvac1, AAVrh.10hAPOE2, ABBV-8E12, ABvac40, AD-35, aducanumab, aflibercept, AGB101, AL002, AL003, allopregnanolone, amlopidine, AMX0035, ANAVEX 2-73, APH-1105, AR1001, AstroStem, atorvastatin, AVP-786, AXS-05, BAC, benfotiamine, BHV4157, BI425809, BIIB092, BIIP06, bioactive dietary polyphenol preparation, BPN14770, brexpiprazole, brolucizumab, byrostatin, CAD106, candesartan, CERE-110, cilostazol, CKD-355, CNP520, COR388, crenezumab, cromolyn, CT1812, curcumin, dabigatran, DAOI, dapagliflozin, deferiprone, DHA, DHP1401, DNL747, dronabinol, efavirenz, elderberry juice, elenbecestat, escitalopram, formoterol, gantenerumab, Ginkgo biloba, grapeseed extract, GRF6019, guanfacine, GV1001, hUCB-MSCs, ibuprofen, icosapent ethyl, ID1201, insulin aspart, insulin glulisine, IONIS MAPTRx, J147, JNJ-63733657, lactulose, lactitol, lemborexant, leuprolide acetate depot, levetiracetam, liraglutide, lithium, LM11A-31-BHS, losartan, L-serine, L-ornithine phenylacetate, Lu AF20513, LY3002813, LY3303560, LY3372993, masitinib, methylene blue, methylphenidate, metronidazole, mirtazapine, ML-4334, MLC901, montelukast, MP-101, nabilone, NDX-1017, neflamapimod, neomycin, nicotinamide, nicotine, nilotinib, nitazoxanide, NPT08, octagam 10%, octohydroaminoacridine succinate, omega-3 PUFA, perindopril, pimavanserin, piromelatine, posiphen, prazosin, PTI-125, ranibizumab rasagiline, rifaximin, riluzole, R07105705, RPh201, sagramostim, salsalate, S-equol, sodium benzoate, sodium phenylacetate, solanezumab, SUVN-502, telmisartan, TEP, THN201, TPI-287, traneurocin, TRx0237, UB-311, valacyclovir, venlafaxine hMSCs (human mesenchymal stem cells), vorinostat, xanamem, zolpidem, or any combination thereof.

In some examples, the pharmaceutical composition can be formulated in unit dose forms or multiple-dose forms. In some examples, the unit dose forms can be physically discrete units suitable for administration to human or non-human subjects (e.g., animals). In some examples, the unit dose forms can be packaged individually. In some examples, each unit dose contains a predetermined quantity of an active ingredient(s) that can be sufficient to produce the desired therapeutic effect in association with pharmaceutical carriers, diluents, excipients, or any combination thereof. In some examples, the unit dose forms comprise ampules, syringes, or individually packaged tablets and capsules, or any combination thereof. In some instances, a unit dose form can be comprised in a disposable syringe. In some instances, unit-dosage forms can be administered in fractions or multiples thereof. In some examples, a multiple-dose form comprises a plurality of identical unit dose forms packaged in a single container, which can be administered in segregated a unit dose form. In some examples, multiple dose forms comprise vials, bottles of tablets or capsules, or bottles of pints or gallons. In some instances, a multiple-dose forms comprise the same pharmaceutically active agents. In some instances, a multiple-dose forms comprise different pharmaceutically active agents.

In some examples, the pharmaceutical composition comprises a pharmaceutically acceptable excipient. In some examples, the excipient comprises a buffering agent, a cryopreservative, a preservative, a stabilizer, a binder, a compaction agent, a lubricant, a chelator, a dispersion enhancer, a disintegration agent, a flavoring agent, a sweetener, or a coloring agent, or any combination thereof.

In some examples, an excipient comprises a buffering agent. In some examples, the buffering agent comprises sodium citrate, magnesium carbonate, magnesium bicarbonate, calcium carbonate, calcium bicarbonate, or any combination thereof. In some examples, the buffering agent comprises sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, or calcium hydroxide and other calcium salts, or any combination thereof.

In some examples, an excipient comprises a cryopreservative. In some examples, the cryopreservative comprises DMSO, glycerol, polyvinylpyrrolidone (PVP), or any combination thereof. In some examples, a cryopreservative comprises a sucrose, a trehalose, a starch, a salt of any of these, a derivative of any of these, or any combination thereof. In some examples, an excipient comprises a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. In some examples, an excipient comprises a surfactant, a sugar, an amino acid, an antioxidant, a salt, a non-ionic surfactant, a solubilizer, a triglyceride, an alcohol, or any combination thereof. In some examples, an excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. In some examples, the excipient can be an excipient described in the Handbook of Pharmaceutical Excipients, American Pharmaceutical Association (1986).

In some examples, the excipient comprises a preservative. In some examples, the preservative comprises an antioxidant, such as alpha-tocopherol and ascorbate, an antimicrobial, such as parabens, chlorobutanol, and phenol, or any combination thereof. In some examples, the antioxidant comprises EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol or N-acetyl cysteine, or any combination thereof. In some examples, the preservative comprises validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitors, or any combination thereof.

In some examples, the excipient comprises a binder. In some examples, the binder comprises starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, or any combination thereof.

In some examples, the binder can be a starch, for example a potato starch, corn starch, or wheat starch; a sugar such as sucrose, glucose, dextrose, lactose, or maltodextrin; a natural and/or synthetic gum; a gelatin; a cellulose derivative such as microcrystalline cellulose, hydroxypropyl cellulose, hydroxyethyl cellulose, hydroxypropyl methyl cellulose, carboxymethyl cellulose, methyl cellulose, or ethyl cellulose; polyvinylpyrrolidone (povidone); polyethylene glycol (PEG); a wax; calcium carbonate; calcium phosphate; an alcohol such as sorbitol, xylitol, mannitol, or water, or any combination thereof.

In some examples, the excipient comprises a lubricant. In some examples, the lubricant comprises magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, or light mineral oil, or any combination thereof. In some examples, the lubricant comprises metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate or talc or a combination thereof.

In some examples, the excipient comprises a dispersion enhancer. In some examples, the dispersion enhancer comprises starch, alginic acid, polyvinylpyrrolidones, guar gum, kaolin, bentonite, purified wood cellulose, sodium starch glycolate, isomorphous silicate, or microcrystalline cellulose, or any combination thereof as high HLB emulsifier surfactants.

In some examples, the excipient comprises a disintegrant. In some examples, a disintegrant comprises a non-effervescent disintegrant. In some examples, a non-effervescent disintegrants comprises starches such as corn starch, potato starch, pregelatinized and modified starches thereof, sweeteners, clays, such as bentonite, micro-crystalline cellulose, alginates, sodium starch glycolate, or gums such as agar, guar, locust bean, karaya, pectin, and tragacanth, or any combination thereof. In some examples, a disintegrant comprises an effervescent disintegrant. In some examples, a suitable effervescent disintegrant comprises bicarbonate in combination with citric acid, and sodium bicarbonate in combination with tartaric acid.

In some examples, the excipient comprises a sweetener, a flavoring agent or both. In some examples, a sweetener comprises glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like, or any combination thereof. In some cases, flavoring agents incorporated into a composition comprise synthetic flavor oils and flavoring aromatics; natural oils; extracts from plants, leaves, flowers, and fruits; or any combination thereof. In some embodiments, a flavoring agent comprises a cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot, or any combination thereof.

In some examples, the excipient comprises a pH agent (e.g., to minimize oxidation or degradation of a component of the composition), a stabilizing agent (e.g., to prevent modification or degradation of a component of the composition), a buffering agent (e.g., to enhance temperature stability), a solubilizing agent (e.g., to increase protein solubility), or any combination thereof. In some examples, the excipient comprises a surfactant, a sugar, an amino acid, an antioxidant, a salt, a non-ionic surfactant, a solubilizer, a triglyceride, an alcohol, or any combination thereof. In some examples, the excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerine, xanthan rubber, soy isoflavones, polysorbate 80, ethyl alcohol, water, teprenone, or any combination thereof. In some examples, the excipient comprises a cryo-preservative. In some examples, the excipient comprises DMSO, glycerol, polyvinylpyrrolidone (PVP), or any combination thereof. In some examples, the excipient comprises a sucrose, a trehalose, a starch, a salt of any of these, a derivative of any of these, or any combination thereof.

In some examples, the pharmaceutical composition comprises a diluent. In some examples, the diluent comprises water, glycerol, methanol, ethanol, or other similar biocompatible diluents, or any combination thereof. In some examples, a diluent comprises an aqueous acid such as acetic acid, citric acid, maleic acid, hydrochloric acid, phosphoric acid, nitric acid, sulfuric acid, or any combination thereof. In some examples, a diluent comprises an alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some examples, the pharmaceutical composition comprises a carrier. In some examples, the carrier comprises a liquid or solid filler, solvent, or encapsulating material. In some examples, the carrier comprises additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), alone or in combination.

In some examples, the pharmaceutical composition can be administered to a subject by any means which will contact the gRNA and/or ADAR (or a vector encoding the gRNA and/or ADAR) with a target cell. In some examples, the specific route will depend upon certain variables such as the target cell and can be determined by the skilled practitioner. In some examples, the pharmaceutical composition can be administered by intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, or direct injection into a tissue, or any combination thereof. In some examples, the target cells can be in or near a tumor and administration can be by direct injection into the tumor or tissue surrounding the tumor. In some examples, the tumor can be a breast tumor and administration comprises impregnation of a catheter and direct injection into the tumor. In some examples, aerosol (inhalation) delivery can be performed using methods known in the art, such as methods described in, for example, Stribling et al., Proc. Natl. Acad. Sci. USA 189: 11277-11281, 1992. In some examples, oral delivery can be performed by complexing an engineered guide (or a vector encoding an engineered guide) to a carrier capable of withstanding degradation by digestive enzymes in the gut of an animal. Examples of such carriers, include plastic capsules or tablets, such as those known in the art.

In some examples, direct injection techniques can be used for administering the gRNA and/or ADAR (or a vector encoding the gRNA and/or ADAR) to a cell or tissue that can be accessible by surgery, and on or near the surface of the body. In some examples, administration of a composition locally within the area of a target cell comprises injecting the composition centimeters and preferably, millimeters from the target cell or tissue.

The appropriate dosage and treatment regimen for the methods of treatment described herein vary with respect to the particular disease being treated, the gRNA and/or ADAR (or a vector encoding the gRNA and/or ADAR) being delivered, and the specific condition of the subject. In some examples, the administration can be over a period of time until the desired effect (e.g., reduction in symptoms is achieved). In some examples, administration can be 1, 2, 3, 4, 5, 6, or 7 times per week. In some examples, administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. In some examples, administration can be over a period of 1, 2, 3, 4, 5, 6, 7, 8, 9, or 10 weeks. In some examples, administration can be over a period of 2, 3, 4, 5, 6 or more months. In some examples, administration can be performed repeatedly over a lifetime of a subject, such as once a month or once a year for the lifetime of a subject. In some examples, administration can be performed repeatedly over a substantial portion of a subject's life, such as once a month or once a year for at least about 1 year, 5 years, 10 years, 15 years, 20 years, 25 years, 30 years, or more. In some examples, treatment can be resumed following a period of remission.

### Kits

Disclosed herein are kits comprising guide RNAs, polynucleotides encoding the same, compositions, pharmaceutical compositions, and isolated cells disclosed herein. In some examples, a kit comprises one or more guide RNAs, polynucleotides encoding the same, compositions, pharmaceutical compositions, or isolated cells disclosed herein and a container. In some examples, the kit comprises a pharmaceutical composition disclosed herein, which comprises an engineered guide RNA disclosed herein or a polynucleotide encoding the engineered guide RNA disclosed herein and a pharmaceutically acceptable excipient, carrier, or diluent. In some examples, the kit comprises one or more delivery vectors disclosed herein which comprise the polynucleotide encoding the engineered guide RNA. In some examples, the kit comprises one or more isolated cells described herein. In some instances, the container can be plastic, glass, metal, or any combination thereof. In some examples, the container can be compartmentalized to receive one or more containers such as vials, tubes, and the like, each of the container(s) comprising one of the separate elements to be used in a method described herein. In some example, the container can be a bottle, a vial, a syringe, or a test tube.

In some examples, in addition to the composition, pharmaceutical composition, or isolated cell disclosed herein, a kit disclosed herein further comprises an additional therapeutic agent disclosed herein. In some examples, the additional therapeutic agent comprises a vascular endothelial growth factor (VEGF) inhibitor, a stem cell treatment, or a vitamin or modified form thereof, or any combination thereof.

In some examples, a kit comprises instructions for use, such as instructions for administration to a subject in need thereof.

In some instances, the kit comprises packaging for a composition or pharmaceutical composition described herein. In some examples, the packaging can be properly labeled. In some instances, the pharmaceutical composition described herein can be manufactured according to good manufacturing practice (cGMP) and labeling regulations.

Also disclosed herein are methods of making a kit disclosed herein. In some examples methods of making the kits herein comprises contacting any of the engineered guides, compositions, pharmaceutical compositions, isolated cells, or isolated plurality of cells disclosed herein with a container. In some examples, methods of making a kit disclosed herein comprising placing an engineered guide RNA, composition, pharmaceutical composition or isolated cell or plurality of cells disclosed herein in a container disclosed herein. In some examples, such methods further comprise placing instructions for use in the container.

### Definitions

Unless defined otherwise, all terms of art, notations and other technical and scientific terms or terminology used herein may be intended to have the same meaning as may be commonly understood by one of ordinary skill in the art to which the claimed subject matter pertains. In some cases, terms with commonly understood meanings may be defined herein for clarity and/or for ready reference, and the inclusion of such definitions herein should not necessarily be construed to represent a substantial difference over what may be generally understood in the art.

Throughout this application, various embodiments may be presented in a range format. It should be understood that the description in range format may be merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “about” a number can refer to that number plus or minus 10% of that number.

As disclosed herein, a “bulge” refers to the structure substantially formed only upon formation of the guide-target RNA scaffold, where contiguous nucleotides in either the engineered guide RNA or the target RNA are not complementary to their positional counterparts on the opposite strand. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a “mismatch.” Further, where the number of participating nucleotides on either the guide RNA side or the target RNA side exceeds 4, the resulting structure is no longer considered a bulge, but rather, is considered an “internal loop.” A “symmetrical bulge” refers to a bulge where the same number of nucleotides is present on each side of the bulge. An “asymmetrical bulge” refers to a bulge where a different number of nucleotides are present on each side of the bulge.

“Canonical amino acids” refer to those 20 amino acids that occur in nature, including for example, the amino acids shown in TABLE 4.

TABLE 4
Naturally occurring amino acids indicated with the three letter
abbreviations, one letter abbreviations, structures, and corresponding codons
Non-polar, aliphatic residues

| | | | | |
|---|---|---|---|---|
| Glycine | Gly | G | 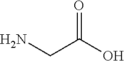 | GGU GGC GGA GGG |
| Alanine | Ala | A | 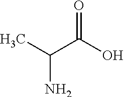 | GCU GCC GCA GCG |
| Valine | Val | V | 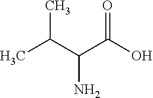 | GUU GUC GUA GUG |
| Leucine | Leu | L | 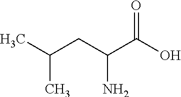 | UUA UUG CUU CUC CUA CUG |
| Isoleucine | Ile | I | 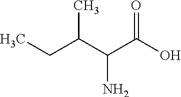 | AUU AUC AUA |
| Proline | Pro | P | 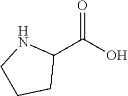 | CCU CCC CCA CCG |

Aromatic residues

| | | | | |
|---|---|---|---|---|
| Phenylalanine | Phe | F | 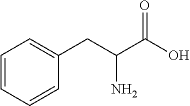 [SMILES: NC(Cc1ccccc1)C(=O)O] | UUU UUC |
| Tyrosine | Tyr | Y | 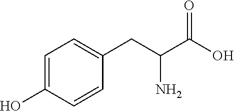 [SMILES: NC(Cc1ccc(O)cc1)C(=O)O] | UAU UAC |
| Tryptophan | Trp | W | 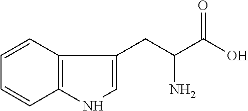 [SMILES: NC(Cc1c[nH]c2ccccc12)C(=O)O] | UGG |

Polar, non-charged residues

| | | | | |
|---|---|---|---|---|
| Serine | Ser | S | 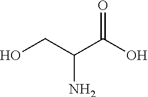 | UCU UCC UCA UCG AGU AGC |
| Threonine | Thr | T | 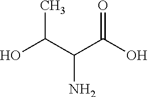 | ACU ACC ACA ACG |
| Cysteine | Cys | C | 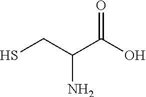 | UGU UGC |
| Methionine | Met | M | 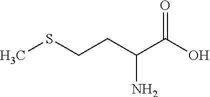 | AUG |
| Asparagine | Asn | N | 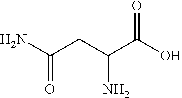 | AAU AAC |
| Glutamine | Gln | Q | 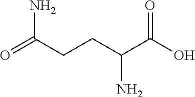 | CAA CAG |

Positively charged residues

| | | | | |
|---|---|---|---|---|
| Lysine | Lys | K | 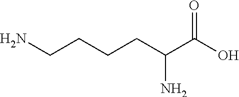 | AAA AAG |
| Arginine | Arg | R | 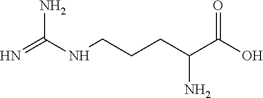 [SMILES: N=C(N)NCCCC(N)C(=O)O] | CGU CGC CGA CGG AGA AGG |
| Histidine | His | H | 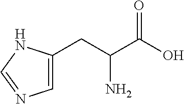 [SMILES: NC(Cc1cnc[nH]1)C(=O)O] | CAU CAC |

Negatively charged residues

| | | | | |
|---|---|---|---|---|
| Aspartate | Asp | D | 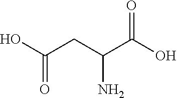 | GAU GAC |
| Glutamate | Glu | E | 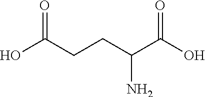 | GAA GAG |

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (e.g., stringent hybridization conditions). Nucleic acids can include nonspecific sequences. As used herein, the term “nonspecific sequence” or “not specific” can refer to a nucleic acid sequence that contains a series of residues that can be not designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element may be present or not (for example, detection). These terms can include quantitative, qualitative or quantitative and qualitative determinations. Assessing can be relative or absolute. “Detecting the presence of” can include determining the amount of something present in addition to determining whether it may be present or absent depending on the context.

The term “encode,” as used herein, refers to an ability of a polynucleotide to provide information or instructions sequence sufficient to produce a corresponding gene expression product. In a non-limiting example, mRNA can encode a polypeptide during translation, whereas DNA can encode an mRNA molecule during transcription.

An “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “facilitates RNA editing” by an engineered guide RNA refers to the ability of the engineered guide RNA when associated with an RNA editing entity and a target RNA to provide a targeted edit of the target RNA by the RNA edited entity. In some instances, the engineered guide RNA can directly recruit or position/orient the RNA editing entity to the proper location for editing of the target RNA. In other instances, the engineered guide RNA when hybridized to the target RNA forms a guide-target RNA scaffold with one or more structural features as described herein, where the guide-target RNA scaffold with structural features recruits or positions/orients the RNA editing entity to the proper location for editing of the target RNA.

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

As disclosed herein, a “hairpin” includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion.

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed.

In some cases, the identity between a reference sequence (query sequence, e.g., a sequence of the disclosure) and a subject sequence, also referred to as a global sequence alignment, can be determined using the FASTDB computer program based son the algorithm of Brutlag et al. (Comp. App. Biosci. 6:237-245 (1990)). In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

In some cases, the identity between a reference sequence (query sequence, e.g., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

As disclosed herein, an “internal loop” refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a “bulge” or a “mismatch,” depending on the size of the structural feature. A “symmetrical internal loop” is formed when the same number of nucleotides is present on each side of the internal loop. An “asymmetrical internal loop” is formed when a different number of nucleotides is present on each side of the internal loop.

“Latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a “bulge” or an “internal loop,” depending on the size of the structural feature.

The term “mutation” as used herein, can refer to an alteration to a nucleic acid sequence or a polypeptide sequence that can be relative to a reference sequence. A mutation can occur in a DNA molecule, an RNA molecule (e.g., tRNA, mRNA), or in a polypeptide or protein, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. Non-limiting examples of mutations in a nucleic acid sequence that, without the mutation, encodes for a polypeptide sequence, include: “missense” mutations that can result in the substitution of one codon for another, a “nonsense” mutations that can change a codon from one encoding a particular amino acid to a stop codon (which can result in truncated translation of proteins), or a “silent” mutations that can be those which have no effect on the resulting protein. The mutation can be a “point mutation,” which can refer to a mutation affecting only one nucleotide in a DNA or RNA sequence. The mutation can be a “splice site mutations,” which can be present pre-mRNA (prior to processing to remove introns) resulting in mistranslation and often truncation of proteins from incorrect delineation of the splice site. The mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (e.g., copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A mutation can comprise a single nucleotide variation (SNV). A mutation can comprise a sequence variant, a sequence variation, a sequence alteration, or an allelic variant.

A presence or an absence of a mutation can indicate an increased risk to develop a disease or condition. A presence or an absence of a mutation can indicate a presence of a disease or condition. A mutation can be present in a benign tissue. Absence of a mutation can indicate that a tissue or sample can be benign. As an alternative, absence of a mutation may not indicate that a tissue or sample can be benign. Methods as described herein can comprise identifying a presence of a mutation in a sample.

The terms “polynucleotide” and “oligonucleotide” can be used interchangeably and can refer to a polymeric form of nucleotides of any length, either deoxyribonucleotides or ribonucleotides or analogs thereof. Polynucleotides can have any three-dimensional structure and can perform any function, known or unknown. The following may be non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term can also refer to both double- and single-stranded molecules. Unless otherwise specified or required, any embodiment of this disclosure that can be a polynucleotide encompasses both the double-stranded form and each of two complementary single-stranded forms known or predicted to make up the double-stranded form.

A polynucleotide can be composed of a specific sequence of nucleotides. A nucleotide comprises a nucleoside and a phosphate group. A nucleotide comprises a sugar (e.g., ribose or 2′deoxyribose) and a nucleobase, such as a nitrogenous base. Non-limiting examples of nucleobases include adenine (A), cytosine (C), guanine (G), thymine (T), uracil (U), and inosine (I). In some embodiments, I can be formed when hypoxanthine can be attached to ribofuranose via a P-N9-glycosidic bond, resulting in the chemical structure:

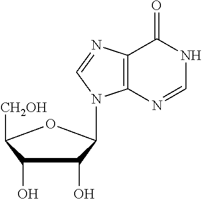

Some polynucleotide embodiments refer to a DNA sequence. In some embodiments, the DNA sequence can be interchangeable with a similar RNA sequence. Some embodiments refer to an RNA sequence. In some embodiments, the RNA sequence can be interchangeable with a similar DNA sequence. In some embodiments, Us and Ts can be interchanged in a sequence provided herein.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together—optionally to preserve the conformation of the fused protein domains, prevent unfavorable interactions between the fused protein domains which can compromise their respective functions, or both.

The term “stop codon” can refer to a three nucleotide contiguous sequence within messenger RNA that signals a termination of translation. Non-limiting examples include in RNA, UAG (amber), UAA (ochre), UGA (umber, also known as opal) and in DNA TAG, TAA or TGA. Unless otherwise noted, the term can also include nonsense mutations within DNA or RNA that introduce a premature stop codon, causing any resulting protein to be abnormally shortened.

The term “structured motif” refers to a combination of two or more structural features in a guide-target RNA scaffold.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject is not necessarily diagnosed or suspected of being at high risk for the disease

The term “in vivo” refers to an event that takes place in a subject's body.

The term “ex vivo” refers to an event that takes place outside of a subject's body. An ex vivo assay may not be performed on a subject. Rather, it can be performed upon a sample separate from a subject. An example of an ex vivo assay performed on a sample can be an “in vitro” assay.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly pair. For example, a wobble base pair can refer to a G paired with a U.

The term “substantially forms” as described herein, when referring to a particular secondary or tertiary structure, refers to formation of at least 80% of the structure under physiological conditions (e.g. physiological pH, physiological temperature, physiological salt concentration, etc.).

As used herein, the terms “treatment” or “treating” can be used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or amelioration of one or more symptoms of an underlying disorder being treated. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of one or more symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

The section headings used herein is for organizational purposes only and is not to be construed as limiting the subject matter described.

### NUMBERED EMBODIMENTS

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.
1. A method of treating a disease or a condition associated with a point mutation in a target molecule encoding a SERPINA1 protein in an individual in need thereof, the method comprising a) administering to the individual an engineered guide exogenous to the individual, the engineered guide comprising at least one recruiting domain for an RNA editing enzyme, wherein the recruiting domain recruits an RNA editing entity and facilitates a chemical modification of a base of a nucleotide in the target RNA molecule encoding the SERPINA1 protein by the RNA editing entity. 2. A method of delivering an engineered guide to a cell, the method comprising a) administering to the individual an engineered guide exogenous to the individual, the engineered guide comprising at least one recruiting domain for an RNA editing enzyme, wherein the recruiting domain recruits an RNA editing entity and facilitates a chemical modification of a base of a nucleotide in a target RNA molecule encoding a SERPINA1 protein by the RNA editing entity. 3. The method of enumerated embodiments 1 or 2, wherein the chemical modification of the base of the nucleotide in the target RNA molecule by the RNA editing entity can be confirmable by an in vitro ELISA assay. 4. The method of any of enumerated embodiments 1-3, wherein the recruiting domain comprises a structural feature. 5. The method of enumerated embodiment 4, wherein the structural feature comprises a bulge, an internal loop, a hairpin or any combination thereof 6. The method of enumerated embodiments 4 or 5, wherein the structural feature can be a hairpin. 7. The method of any one of enumerated embodiments 1-6, wherein the engineered guide comprises DNA. 8. The method of any one of enumerated embodiments 1-6, wherein the engineered guide comprises RNA. 9. The method of any one of any one of enumerated embodiments 1-8, wherein the engineered guide comprises at least about 85% sequence identity to a nucleic acid sequence provided in SEQ ID NOS: 2-5. 10. The method of any one of enumerated embodiments 1-9, wherein the RNA editing entity comprises: an Adenosine deaminase acting on RNA (ADAR) or an Apolipoprotein B mRNA Editing Catalytic Polypeptide-like (APOBEC) enzyme; a catalytically active fragment of (a); fusion polypeptide comprising (a) or (b); or any combination of these. 11. The method of any one of enumerated embodiments 1-10, wherein the RNA editing entity can be endogenous to a cell. 12. The method of any one of enumerated embodiments 1-11, wherein the RNA editing entity comprises ADAR. 13. The method of enumerated embodiment 12, wherein the ADAR comprises human ADAR (hADAR). 14. The method of enumerated embodiments 12 or 13, wherein the ADAR comprises ADAR1 or ADAR2. 15. The method of any one of enumerated embodiments 1-14, wherein the engineered guide can be encoded for by a polynucleotide comprised in or on a first delivery vector, or wherein a polynucleotide comprised in a second delivery vector encodes at least in part the engineered guide. 16. The method of any one of enumerated embodiments 1-15, wherein the first delivery vector or the second delivery vector comprises an adeno-associated viral (AAV) vector or derivatives thereof 17. The method of enumerated embodiment 16, wherein the AAV vector can be from an adeno-associated virus having a serotype selected from AAV1, AAV2, AAV3, AAV4, AAV5, AAV6, AAV7, AAV8, AAV9, AAV10, or AAV 11. 18. The method of any one of enumerated embodiments 16-17, wherein said AAV vector can be a recombinant AAV (rAAV) vector, a hybrid AAV vector, a chimeric AAV vector, a self-complementary AAV (scAAV) vector, a single-stranded AAV or any combination thereof 19. The method of any one of enumerated embodiments 16-18, wherein the AAV vector comprises a genome comprising a replication gene and inverted terminal repeats from a first AAV serotype and a capsid protein from a second AAV serotype. 20. The method of any one of enumerated embodiments 16-19, wherein the AAV vector can be an AAV 2/5 vector, an AAV 2/6 vector, an AAV 2/7 vector, an AAV2/8 vector, or an AAV 2/9 vector. 21. The method of any one of enumerated embodiments 1-20, wherein the engineered guide, the first delivery vector, or the second delivery vector can be comprised in a pharmaceutical composition in unit dosage form comprising at least one pharmaceutically acceptable: excipient, carrier, or diluent. 22. The method of enumerated embodiment 21, wherein the pharmaceutical composition can be administered at a therapeutically effective dose to treat the subject. 23. The method of enumerated embodiments 21 or 22, wherein the pharmaceutical composition can be administered to the subject intrathecally, intraocularly, intravitreally, retinally, intravenously, intramuscularly, intraventricularly, intracerebrally, intracerebellarly, intracerebroventricularly, intraperenchymally, subcutaneously, or a combination thereof 24. The method of any one of enumerated embodiments 1-23, wherein subject can be diagnosed with Alpha-1 antitrypsin deficiency. 25. The method of enumerated embodiment 24, wherein the subject can be diagnosed with the disease or the condition by an in vitro assay. 26. The method of any one of enumerated embodiments 1-25, further comprising administering an additional therapeutic agent for the treatment of the disease or the condition. 27. The method of enumerated embodiment 26, wherein the additional therapeutic comprises an ammonia reducer, a beta blocker, a synthetic hormone, an antibiotic, or an antiviral drug, or a combination thereof, for the treatment of a liver disease or disorder. 28. The method of enumerated embodiment 26, wherein the additional therapeutic comprises a vascular endothelial growth factor (VEGF) inhibitor, a stem cell treatment, a vitamin or modified form thereof, for the treatment of macular degeneration. 29. An engineered guide comprising: (a) at least one RNA-editing enzyme recruiting domain; (b) at least one nucleic acid structural feature; and wherein the engineered guide can be configured to facilitate editing of a nucleotide base of a nucleotide of a target RNA molecule to modulate an expression level of an SERPINA1 protein expressed from said target RNA molecule. 30. The engineered guide of enumerated embodiment 29, wherein the target RNA molecule can be an mRNA molecule. 31. The engineered guide of enumerated embodiment 29, wherein the target RNA molecule can be a pre-mRNA molecule. 32. The engineered guide of any one of enumerated embodiments 29-31, wherein the structural feature comprises a bulge, an internal loop, a hairpin or any combination thereof 33. The engineered guide of any one of enumerated embodiments 29-32, wherein the structural feature can be a hairpin. 34. The engineered guide of any one of enumerated embodiments 29-33, wherein the engineered guide comprises DNA. 35. The engineered guide of any one of enumerated embodiments 29-34, wherein the engineered guide comprises RNA. 36. The engineered guide of any one of any one of enumerated embodiments 29-35, wherein the engineered guide comprises at least about 85% sequence identity to a nucleic acid sequence provided in SEQ ID NOS: 2-5. 37. The method of any one of enumerated embodiments 29-36, wherein the RNA editing entity comprises: an Adenosine deaminase acting on RNA (ADAR) or an Apolipoprotein B mRNA Editing Catalytic Polypeptide-like (APOBEC) enzyme; a catalytically active fragment of (a); fusion polypeptide comprising (a) or (b); or any combination of these. 38. A composition comprising the engineered guide of any one of enumerated embodiments 1-37. 39. A polynucleotide at least partially encoding the engineered guide of any one of enumerated embodiments 1-37. 40. A delivery vector, and optionally a second delivery vector, comprising the engineered guide of any one of enumerated embodiments 1-37 or the polynucleotide of enumerated embodiment 39. 41. The delivery vector of enumerated embodiment 40, wherein the delivery vector or the second delivery vector, comprises a viral vector. 42. The delivery vector of enumerated embodiment 40, wherein the delivery vector or the second delivery vector comprises an adeno-associated viral (AAV) vector or derivatives thereof 43. The deliver vector of enumerated embodiment 42, wherein the AAV vector can be from an adeno-associated virus having a serotype selected from AAV1, AAV2, AAV3, AAV4, AAV5, AAV6, AAV7, AAV8, AAV9, AAV10, or AAV 11. 44. The delivery vector of enumerated embodiment 42 or 43, wherein said AAV vector can be a recombinant AAV (rAAV) vector, a hybrid AAV vector, a chimeric AAV vector, a self-complementary AAV (scAAV) vector, or any combination thereof. 45. The delivery vector of any one of enumerated embodiments 42-44, wherein the AAV vector comprises a genome comprising a replication gene and inverted terminal repeats from a first AAV serotype and a capsid protein from a second AAV serotype. 46. The delivery vector of any one of enumerated embodiments 42-45, wherein the AAV vector can be an AAV 2/5 vector, an AAV 2/6 vector, an AAV 2/7 vector, an AAV2/8 vector, or an AAV 2/9 vector. 47. An isolated cell comprising the engineered guide of any one of enumerated embodiments 1-46. 48. An isolated cell comprising the delivery vector of any one of enumerated embodiments 40-46. 49. The isolated cell of enumerated embodiment 47 or 48, wherein the isolated cell can be an immune cell. 50. The isolated cell of enumerated embodiment 48 or 49, wherein the immune cell can be a T cell. 51. An isolated plurality of cells comprising engineered guide of any one of enumerated embodiments 1-50 or the vector of any one of enumerated embodiments 40-46. The isolated plurality of cells of enumerated embodiment 51, wherein the immune cells can be T cells.

### Additional Embodiments

1. An engineered guide that is configured, upon hybridization to a target RNA molecule, to form a double stranded substrate with at least a portion of the target RNA molecule, (i) wherein the double stranded substrate comprises at least one structural feature comprising a bulge, internal loop, hairpin, or any combination thereof; (ii) wherein the double stranded substrate recruits an RNA editing entity; and, wherein the RNA editing entity facilitates a chemical modification of a base of a nucleotide in the target RNA molecule by the RNA editing entity. 2. The engineered guide of embodiment 1, wherein the chemical modification of the base of the nucleotide in the target RNA molecule by the RNA editing entity is confirmable by Sanger sequencing, next generation sequencing, or a combination thereof 3. The engineered guide of embodiment 1 or 2, wherein the engineered guide is single-stranded. 4. The engineered guide of any one of embodiments 1-3, wherein the double stranded substrate comprises a structured motif comprising two or more of a structural feature. 5. The engineered guide of any one of embodiments 1-4, wherein the structural feature comprises a bulge, an internal loop, a hairpin, a mismatch, a wobble base pair, or any combination thereof. 6. The engineered guide of any one of embodiments 1-6, wherein the structural feature comprises a bulge. 7. The engineered guide of any one of embodiments 5-6, wherein the bulge comprises an asymmetric bulge. 8. The engineered guide of any one of embodiments 5-7, wherein the bulge comprises a symmetric bulge. 9. The engineered guide of any one of embodiments 5-8, wherein the bulge comprises about 1 to about 4 nucleotides of the engineered guide and about 0 to about 4 nucleotides of the target RNA molecule. 10. The engineered guide of any one of embodiments 5-9, wherein the bulge comprises about 0 to about 4 nucleotides of the engineered guide and about 1 to about 4 nucleotides of the target RNA molecule. 11. The engineered guide of any one of embodiments 5-10, wherein the bulge comprises 3 nucleotides of the engineered guide and 3 nucleotides of the target RNA molecule. 12. The engineered guide of any one of embodiments 1-11, wherein the structural feature comprises an internal loop. 13. The engineered guide of any one of embodiments 5-13, wherein the internal loop comprises an asymmetric internal loop. 14. The engineered guide of any one of embodiments 5-13, wherein the internal loop comprises a symmetric internal loop. 15. The engineered guide of any one of embodiments 5-14, wherein the internal loop is formed by about 5 to about 10 nucleotides of either the engineered guide or the target RNA molecule. 16. The engineered guide of any one of embodiments 5-15, wherein the structural feature comprises a hairpin. 17. The engineered guide of any one of embodiments 5-16, wherein the hairpin comprises a double stranded RNA non-targeting domain. 18. The engineered guide of any of embodiments 5-17, wherein the stem loop of the hairpin comprises about 3 to about 15 nucleotides in length. 19. The engineered guide of any one of embodiments 1-18, wherein the structural feature comprises a mismatch. 20. The engineered guide of any one of embodiments 5-19, wherein the mismatch comprises a base in the engineered guide opposite to and unpaired with a base in the target RNA molecule. 21. The engineered guide of any one of embodiments 5-20, wherein the mismatch comprises a G/G mismatch. 22. The engineered guide of any one of embodiments 5-21, wherein the mismatch comprises an A/C mismatch and wherein the A is in the target RNA molecule and the C is in the engineered guide. 23. The engineered guide of embodiment 21, wherein the A in the A/C mismatch is the base of the nucleotide in the target RNA molecule chemically modified by the RNA editing entity. 24. The engineered guide of any one of embodiments 1-23, wherein the structural feature comprises a wobble base pair. 25. The engineered guide of any one of embodiments 5-24, wherein the wobble base pair comprises a guanine paired with a uracil. 26. The engineered guide of any one of embodiments 5-25, wherein the structural motif comprises two bulges and an A/C mismatch. 27. The engineered guide of any one of embodiments 1-26, wherein the engineered guide, when present in an aqueous solution and not bound to the target RNA molecule, if it binds to the RNA editing entity, does so with a dissociation constant about greater than or equal to 500 nM. 28. The engineered guide of any one of embodiments 1-27, wherein the double stranded substrate comprises a mismatch. 29. The engineered guide of embodiment 28, wherein the mismatch comprises a base in the engineered guide opposite to and unpaired with a base in the target RNA molecule. 30. The engineered guide of embodiment 28, wherein the mismatch comprises an A/C mismatch and wherein the A is in the target RNA molecule and the C is in the engineered guide. 31. The engineered guide of embodiment 30, wherein the A in the A/C mismatch is the base of the nucleotide in the target RNA molecule is chemically modified by the RNA editing entity. 32. The engineered guide of embodiment 31, wherein the engineered guide comprises a C opposite the base of the nucleotide in the target RNA chemically modified by the RNA editing entity. 33. The engineered guide of any one of embodiments 1-32, wherein the target RNA molecule comprises a 5′ G adjacent to the base of the nucleotide in the target RNA chemically modified by the RNA editing entity. 34. The engineered guide of any one of embodiments 1-33, wherein the engineered guide comprises a 5′G adjacent to the C opposite to and unpaired with the A in the target RNA molecule chemically modified by the RNA editing entity. 35. The engineered guide of any one of embodiments 1-34, wherein the engineered guide, when bound to the target RNA molecule, mimics a naturally occurring substrate of an ADAR enzyme. 36. The engineered guide of any one of embodiments 1-35, wherein the engineered guide, when bound to the target RNA molecule, mimics a naturally occurring substrate to a drosophila ADAR enzyme. 37. The engineered guide of any one of embodiments 1-36, wherein the engineered guide comprises at least about 85% sequence identity to a nucleic acid sequence provided in SEQ ID NOS: 1-34 or 55-61. 38. The engineered guide of any one of embodiments 1-37, wherein the RNA editing entity is: (a) ADAR or APOBEC; (b) a catalytically active fragment of (a); (c) fusion polypeptide comprising (a) or (b); or (d) any combination of these. 39. The engineered guide of any one of embodiments 1-38, wherein the RNA editing entity is endogenous to a cell. 40. The engineered guide of any one of embodiments 1-39, wherein the RNA editing entity comprises ADAR. 41. The engineered guide of embodiment 40, wherein the ADAR comprises human ADAR (hADAR). 42. The engineered guide of embodiment 40, wherein the ADAR comprises ADAR1 or ADAR2 43. The engineered guide of any one of embodiments 1-42, wherein the engineered guide comprises modified DNA bases, unmodified DNA bases, or a combination thereof 44. The engineered guide of any one of embodiments 1-42, wherein the engineered guide comprises modified RNA bases, unmodified RNA bases, or a combination thereof 45. The engineered guide of any one embodiments 1-44, wherein the target RNA molecule is an mRNA molecule. 46. The engineered guide of any one of embodiments 1-44, wherein the target RNA molecule is a pre-mRNA molecule. 47. The engineered guide of any one of embodiments 1-44, wherein the engineered guide is isolated, or purified, or both. 48. The engineered guide of any one of embodiments 1-44, wherein the target RNA molecule encodes APP, ABCA4, SERPINA1, HEXA, LRRK2, CFTR, SNCA, Tau, or LIPA, a fragment any of these, or any combination thereof. 49. The engineered guide of any one of embodiments 1-48, wherein the nucleotide of the target RNA molecule is a point mutation in the target RNA molecule relative to an otherwise identical reference target RNA molecule. 50. The engineered guide of embodiment 49, wherein the point mutation comprises a missense mutation. 51. The engineered guide of embodiment 50, wherein the missense mutation results in an A at the mutated nucleotide. 52. The engineered guide of any one of embodiments 49-51, wherein the point mutation facilitates unintended splicing of the target RNA molecule. 53. The engineered guide of embodiment 49, wherein the point mutation comprises a splice site mutation positioned adjacent to a C and a G on a 5′ and a 3′ end of the point mutation, respectively. 54. The engineered guide of any one of embodiments 1-53, wherein the target RNA molecule is encoded by the SERPINA1 gene, or a portion thereof. 55. The engineered guide of embodiment 53, wherein the SERPINA1 gene comprises a substitution of a G with an A at nucleotide position 9989 relative to a wildtype SERPINA1 gene (such as accession number NC_000014.9:c94390654-94376747). 56. The engineered guide of any one of embodiments 1-53, wherein the target RNA molecule is encoded by an ABCA4 gene, or a portion thereof. 57. The engineered guide of embodiment 56, wherein the ABCA4 gene comprises a substitution of a G with an A at nucleotide position 5882 relative to a wildtype ABCA4 gene (such as accession number NC_000001.11:c94121149-93992837). 58. The engineered guide of embodiment 56, wherein the ABCA4 gene comprises a substitution of a G with an A at nucleotide position 5714 relative to a wildtype ABCA4 gene (such as accession number NC_000001.11:c94121149-93992837). 59. The engineered guide of embodiment 56, wherein the ABCA4 gene comprises a substitution of a G with an A at nucleotide position 6320 relative to a wildtype ABCA4 gene (such as accession number NC_000001.11:c94121149-93992837). 60. The engineered guide of any one of embodiments 1-53, wherein the target RNA molecule is encoded by the LRRK2 gene, or a portion thereof 61. The engineered guide of any one of embodiments 1-60, wherein the engineered guide comprises a C opposite the base of the nucleotide in the target RNA chemically modified by the RNA editing entity. 62. The engineered guide of any one of embodiments 1-59, wherein the target RNA molecule comprises a 5′ G adjacent to the base of the nucleotide in the target RNA chemically modified by the RNA editing entity. 63. The engineered guide of any one of embodiments 1-62, wherein the engineered guide comprises a 5′G adjacent to the C opposite to and unpaired with the A in the target RNA molecule chemically modified by the RNA editing entity. 64. A method of delivering an engineered guide to a cell, the method comprising: delivering directly or indirectly to the cell the engineered guide that at least partially hybridizes to and forms a double stranded substrate with at least a portion of a target RNA molecule, (i) wherein the double stranded substrate comprises at least one structural feature comprising a bulge, internal loop, hairpin, or any combination thereof, (ii) wherein the double stranded substrate recruits an RNA editing entity; and, 65. wherein the RNA editing entity facilitates a chemical modification of a base of a nucleotide in the target RNA molecule by the RNA editing entity (ii) A method of treating or preventing a disease or a condition in a subject in need thereof, the method comprising: administering to the subject having the disease or the condition an engineered guide thereby treating or preventing the disease or the condition in the subject, wherein the engineered guide: (a) at least in part associates with at least a portion of a target RNA molecule; (b) in association with the target RNA molecule, forms a double stranded substrate comprising at least one structural feature, and wherein the double stranded substrate recruits an RNA editing entity; and (c) facilitates a chemical modification of a base of a nucleotide in the target RNA molecule by the RNA editing entity. 66. The method of embodiment 64 or 65, wherein the chemical modification of the base of the nucleotide in the target RNA molecule by the RNA editing entity is confirmable by Sanger sequencing, next generation sequencing, or a combination thereof. 67. The methods of any of embodiments 64-66, wherein the engineered guide is single-stranded. 68. The method of any one of embodiments 64-67, wherein the double stranded substrate comprises a structured motif comprising two or more of a structural feature. 69. The method of any one of embodiments 64-68, wherein the structural feature comprises a bulge, an internal loop, a hairpin, a mismatch, a wobble base pair, or any combination thereof. 70. The method of any one of embodiments 64-69, wherein the structural feature comprises a bulge. 71. The method of any one of embodiments 69-70, wherein the bulge comprises an asymmetric bulge. 72. The method of any one of embodiments 69-71, wherein the bulge comprises a symmetric bulge. 73. The method of any one of embodiments 69-72, wherein the bulge comprises about 2 to about 4 nucleotides of the engineered guide and about 2 to about 4 nucleotides of the target RNA molecule. 74. The method of any one of embodiments 69-73, wherein the bulge comprises 3 nucleotides of the engineered guide and 3 nucleotides of the target RNA molecule. 75. The method of any one of embodiments 64-74, wherein the structural feature comprises an internal loop. 76. The method of any one of embodiments 69-75, wherein the internal loop comprises an asymmetric internal loop. 77. The method of any one of embodiments 69-76, wherein the internal loop comprises a symmetric internal loop. 78. The method of any one of embodiments 69-77, wherein the internal loop is formed by about 5 to about 10 nucleotides of either the engineered guide or the target RNA molecule. 79. The method of any one of embodiments 64-78, wherein the structural feature comprises a hairpin. 80. The method of any one of embodiments 69-79, wherein the hairpin comprises a double stranded RNA non-targeting domain. 81. The method of any of embodiments 69-80, wherein the stem loop of the hairpin comprises about 3 to about 15 nucleotides in length. 82. The method of any one of embodiments 69-81, wherein the structural feature comprises a mismatch. 83. The method of any one of embodiments 69-83, wherein the mismatch comprises a base in the engineered guide opposite to and unpaired with a base in the target RNA molecule. 84. The method of any one of embodiments 69-83, wherein the mismatch comprises a G/G mismatch. 85. The method of any one of embodiments 69-84, wherein the mismatch comprises an A/C mismatch and wherein the A is in the target RNA molecule and the C is in the engineered guide. 86. The method of embodiment 85, wherein the A in the A/C mismatch is the base of the nucleotide in the target RNA molecule chemically modified by the RNA editing entity. 87. The method of any one of embodiments 64-86, wherein the structural feature comprises a wobble base pair. 88. The method of any one of embodiments 69-87, wherein the wobble base pair comprises a guanine paired with a uracil. 89. The method of any one of embodiments 69-87, wherein the structural motif comprises two bulges and an A/C mismatch. 90. The method of any one of embodiments 69-87, wherein the engineered guide, when present in an aqueous solution and not bound to the target RNA molecule, if it binds to the RNA editing entity, does so with a dissociation constant of greater than about 500 nM. 91. The method of any one of embodiments 64-91, wherein the engineered guide comprises modified DNA bases, unmodified DNA bases, or a combination thereof 92. The method of any one of embodiments 64-92, wherein the engineered guide comprises modified RNA bases, unmodified RNA bases or a combination thereof 93. The method of any one of embodiments 64-93, wherein the engineered guide comprises at least about 85% sequence identity to a nucleic acid sequence provided in SEQ ID NOS: 1-34 or 55-61. 94. The method of any one of embodiments 64-94, wherein the RNA editing entity comprises: (a) an Adenosine deaminase acting on RNA (ADAR) or an Apolipoprotein B mRNA Editing Catalytic Polypeptide-like (APOBEC) enzyme; (b) a catalytically active fragment of (a); (c) fusion polypeptide comprising (a) or (b); or (d) any combination of these. 95. The method of any one of embodiments 64-95, wherein the RNA editing entity is endogenous to a cell. 96. The method of any one of embodiments 64-97, wherein the RNA editing entity comprises ADAR. 97. The method of embodiment 96, wherein the ADAR comprises human ADAR (hADAR). 98. The method of any one of embodiments 96-97, wherein the ADAR comprises ADAR1 or ADAR2. 99. The method of any one of embodiments 64-99, wherein the target RNA molecule encodes APP, ABCA4, SERPINA1, HEXA, LRRK2, SNCA, CFTR, or LIPA, a fragment of any of these, or any combination thereof. 100. The method of any one of embodiments 64-99, wherein the target RNA molecule encodes at least a portion of a cleavage site of a protein. 101. The method of embodiment 100, wherein a cleavage product of the protein produced by cleavage of the protein at the cleavage site contributes to the pathogenesis or progression of a disease. 102. The method of any one of embodiments 63-101, wherein the target RNA molecule encodes a Beta-secretase cleavage site of an amyloid precursor protein (APP). 103. The method of any one of embodiments 52-102, wherein the nucleotide of the target RNA molecule is a point mutation in the target RNA molecule relative to an otherwise identical reference target RNA molecule. 104. The method of embodiment 103, wherein the point mutation, in combination with two additional nucleotides form a premature stop codon which causes translation termination of an expression product expressed from the target RNA molecule. 105. The method of embodiment 104, wherein the two additional nucleotides are (i) a U and an (ii) A or a G on a 5′ and 3′ end of the point mutation. 106. The method of embodiment 103, wherein the point mutation is a missense mutation. 107. The method of embodiment 106, wherein the missense mutation results in an A at the mutated nucleotide. 108. The method of embodiment 103, wherein the point mutation facilitates unintended splicing of the target RNA molecule. 109. The method of embodiment 103 or 106, wherein the point mutation is a splice site mutation positioned adjacent to a C and a G on a 5′ and a 3′ end of the point mutation, respectively. 110. The method of any one of embodiments 63-109, wherein the target RNA molecule is encoded by the SERPINA1 gene, or a portion thereof. 111. The method of embodiment 110, wherein the SERPINA1 gene comprises a substitution of a G with an A at nucleotide position 9989 relative to a wildtype SERPINA1 gene (such as accession number NC_000014.9:c94390654-94376747). 112. The method of any one of embodiments 63-109, wherein the target RNA molecule is encoded by an ABCA4 gene, or a portion thereof. 113. The method of embodiment 111, wherein the ABCA4 gene comprises a substitution of a G with an A at nucleotide position 5882 relative to a wildtype ABCA4 gene (such as accession number NC_000001.11:c94121149-93992837). 114. The method of embodiment 111, wherein the ABCA4 gene comprises a substitution of a G with an A at nucleotide position 5714 relative to a wildtype ABCA4 gene (such as accession number NC_000001.11:c94121149-93992837). 115. The method of embodiment 111, wherein ABCA4 gene comprises a substitution of a G with an A at nucleotide position 6320 relative to a wildtype ABCA4 gene (such as accession number NC_000001.11:c94121149-93992837). 116. The method of any one of embodiments 63-109, wherein the target RNA molecule is encoded by the LRRK2 gene, or a portion thereof 117. The method of any one of embodiments 63-109, wherein the engineered guide comprises a C opposite the base of the nucleotide in the target RNA chemically modified by the RNA editing entity. 118. The method of any one of embodiments 62-116, wherein the target RNA molecule comprises a 5′ G adjacent to the base of the nucleotide in the target RNA chemically modified by the RNA editing entity. 119. The method of any one of embodiments 62-117, wherein the engineered guide comprises a 5′G adjacent to the C opposite to and unpaired with the A in the target RNA molecule chemically modified by the RNA editing entity. 120. The method of any one of embodiments 63-119, wherein the engineered guide is encoded for by a polynucleotide comprised in or on a first delivery vector, or wherein a polynucleotide comprised in a second delivery vector encodes at least in part the engineered guide. 121. The method of embodiment 119, wherein the first delivery vector or the second delivery vector comprises an adeno-associated viral (AAV) vector or derivatives thereof. 122. The method of embodiment 120, wherein the AAV vector is from an adeno-associated virus having a serotype selected from AAV1, AAV2, AAV3, AAV4, AAV5, AAV6, AAV7, AAV8, AAV9, AAV10, AAV 11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. 123. The method of any one of embodiments 120-121, wherein said AAV vector is a recombinant AAV (rAAV) vector, a hybrid AAV vector, a chimeric AAV vector, a self-complementary AAV (scAAV) vector, a single-stranded AAV or any combination thereof 124. The method of any one of embodiments 120-122, wherein the AAV vector comprises a genome comprising a replication gene and inverted terminal repeats from a first AAV serotype and a capsid protein from a second AAV serotype. 125. The method of any one of embodiments 120-123, wherein the AAV vector is an AAV 2/5 vector, an AAV 2/6 vector, an AAV 2/7 vector, an AAV2/8 vector, or an AAV 2/9 vector. 126. The method of any one of embodiments 123-124, wherein the inverted terminal repeats comprise a 5′ inverted terminal repeat, a 3′ inverted terminal repeat, and a mutated inverted terminal repeat. 127. The method of embodiment 125, wherein the mutated inverted terminal repeat lacks a terminal resolution site. 128. The method of any one of embodiments 62-126, wherein the engineered guide, the first delivery vector, or the second delivery vector is comprised in a pharmaceutical composition in unit dosage form comprising at least one pharmaceutically acceptable: excipient, carrier, or diluent. 129. The method of embodiment 128, wherein the pharmaceutical composition is administered at a therapeutically effective dose to treat the subject. 130. The method of embodiment 127 or 128, wherein the pharmaceutical composition is administered to the subject intrathecally, intraocularly, intravitreally, retinally, intravenously, intramuscularly, intraventricularly, intracerebrally, intracerebellarly, intracerebroventricularly, intraperenchymally, subcutaneously, or a combination thereof 131. The method of any one of embodiments 65-130, wherein the disease is macular degeneration. 132. The method of any one of embodiments 65-131, wherein the disease is Stargardt Disease 133. The method of any one of embodiments 65-132, wherein the disease is alpha-1 antitrypsin deficiency (AATD). 134. The method of any one of embodiments 65-133, wherein the disease or the condition comprises a neurological disease or disorder. 135. The method of embodiment 134, wherein the neurological disease or disorder comprises Parkinson's disease, Alzheimer's disease, a Tauopathy, or dementia. 136. The method of any one of embodiments 65-135, wherein the disease or the condition comprises a liver disease or disorder. 137. The method of embodiment 136, wherein the liver disease or disorder comprises liver cirrhosis. 138. The method of embodiment 136, wherein the liver disease or disorder is alpha-1 antitrypsin deficiency (AAT deficiency). 139. The method of any one of embodiments 65-138, wherein the subject is diagnosed with the disease or the condition. 140. The method of embodiment 139, wherein the subject is diagnosed with the disease or the condition by an in vitro assay. 141. The method of any one of embodiments 65-140, further comprising administering an additional therapeutic agent for the treatment of the disease or the condition. 142. The method of embodiment 141, wherein the additional therapeutic comprises an ammonia reducer, a beta blocker, a synthetic hormone, an antibiotic, or an antiviral drug, or a combination thereof, for the treatment of a liver disease or disorder. 143. The method of embodiment 141, wherein the additional therapeutic comprises a vascular endothelial growth factor (VEGF) inhibitor, a stem cell treatment, a vitamin or modified form thereof, for the treatment of macular degeneration. 144. A composition comprising the engineered guide of any one of embodiments 1-143. 145. A polynucleotide at least partially encoding the engineered guide of any one of embodiments 1-144. 146. A delivery vector, and optionally a second delivery vector, comprising the engineered guide of any one of embodiments 1-143 or the polynucleotide of embodiment 145. 147. The delivery vector of embodiment 146, wherein the delivery vector or the second delivery vector, comprises a viral vector. 148. The delivery vector of embodiment 146, wherein the delivery vector or the second delivery vector comprises an adeno-associated viral (AAV) vector or derivatives thereof 149. The deliver vector of embodiment 147, wherein the AAV vector is from an adeno-associated virus having a serotype selected from AAV1, AAV2, AAV3, AAV4, AAV5, AAV6, AAV7, AAV8, AAV9, AAV10, AAV 11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. 150. The delivery vector of embodiment 147 or 148, wherein said AAV vector is a recombinant AAV (rAAV) vector, a hybrid AAV vector, a chimeric AAV vector, a self-complementary AAV (scAAV) vector, or any combination thereof 151. The delivery vector of any one of embodiments 147-150, wherein the AAV vector comprises a genome comprising a replication gene and inverted terminal repeats from a first AAV serotype and a capsid protein from a second AAV serotype. 152. The delivery vector of any one of embodiments 147-150, wherein the AAV vector is an AAV 2/5 vector, an AAV 2/6 vector, an AAV 2/7 vector, an AAV2/8 vector, or an AAV 2/9 vector. 153. An isolated cell comprising the engineered guide of any one of embodiments 1-143. 154. An isolated cell comprising the delivery vector of any one of embodiments 146-152. 155. The isolated cell of embodiment 153 or 154, wherein the isolated cell is an immune cell. 156. The isolated cell of embodiment 155, wherein the immune cell is a T cell. 157. An isolated plurality of cells comprising engineered guide of any one of embodiments 1-143 or the vector of any one of embodiments 146-152. 158. The isolated plurality of cells of embodiment 157, wherein the immune cells are T cells. 159. A pharmaceutical composition comprising: (a) the engineered guide of any one of embodiments 1-145, the composition of embodiment 146, the isolated cell of any one of embodiments 155-158, or the isolated plurality of cells of embodiment 159 or 160; and (b) a pharmaceutically acceptable: excipient, carrier, or diluent. 160. The pharmaceutical composition of embodiment 159 in unit dose form. 161. The pharmaceutical composition of embodiment 159 or 160, further comprising an additional therapeutic agent. 162. The pharmaceutical composition of embodiment 159, wherein the additional therapeutic agent comprises an ammonia reducer, a beta blocker, a synthetic hormone, an antibiotic, or an antiviral drug, a vascular endothelial growth factor (VEGF) inhibitor, a stem cell treatment, a vitamin or modified form thereof, or any combination thereof 163. A kit comprising: (a) the engineered guide of any one of embodiments 1-143, the composition of embodiment 144, the pharmaceutical composition of any one of embodiments 159-162, the isolated cell of any one of embodiments 153-156, or the isolated plurality of cells of embodiments 157 or 158; and (b) a container. 164. The kit of embodiment 163, further comprising an additional therapeutic agent. 165. The kit of embodiment 164, wherein the additional therapeutic agent comprises an ammonia reducer, a beta blocker, a synthetic hormone, an antibiotic, or an antiviral drug, a vascular endothelial growth factor (VEGF) inhibitor, a stem cell treatment, a vitamin or modified form thereof, or any combination thereof 166. A method of making the kit of embodiment 165, the method comprising: contacting the engineered guide of any one of embodiments 1-143, the composition of embodiment 144, the pharmaceutical composition of any one of embodiments 159-162, the isolated cell of any one of embodiments 153-156, or the isolated plurality of cells of embodiments 157 or 158, with the container. 167. A method of making an expression vector, the method comprising: introducing a transgene at least partially encoding the engineered guide of any one of embodiments 1-143 into an expression vector. 168. A method of making a delivery vector, the method comprising: loading a delivery vector with the engineered guide of any one of embodiments 1-143. 169. A method of generating an AAV delivery vector, the method comprising: (a) introducing into a cell: (i) a polynucleotide encoding the engineered guide of any one of embodiments 1-145; and (ii) a viral genome comprising a Replication (Rep) gene and Capsid (Cap) gene that encodes a wild-type AAV capsid protein or modified version thereof; (b) expressing in the cell the wild-type AAV capsid protein or modified version thereof; (c) assembling an AAV particle; and (d) packaging the polynucleotide encoding the engineered guide RNA in the AAV particle, thereby generating an AAV delivery vector. 170. The method of embodiment 169, wherein the polynucleotide encoding the engineered guide is enclosed by a 5′ and a 3′ inverted terminal repeat (ITR) sequence. 171. The method of embodiment 170, wherein the viral genome further comprises a 5′ ITR and a 3′ ITR. 172. The method of embodiment 170, wherein the polynucleotide encoding the engineered guide is comprised in a plasmid. 173. The method any one of embodiments 169-172, wherein the method further comprises introducing into the cell a helper plasmid comprising helper genes isolated from an adenovirus. 174. The method any one of embodiments 169-173, wherein the capsid proteins, the Rep gene, or the ITR sequences, or a combination thereof are from an adeno-associated virus having a serotype selected from AAV1, AAV2, AAV3, AAV4, AAV5, AAV6, AAV7, AAV8, AAV9, AAV10, AAV 11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. 175. The method of embodiment 175, wherein said AAV delivery vector is a recombinant AAV (rAAV) vector, a hybrid AAV vector, a chimeric AAV vector, a self-complementary AAV (scAAV) vector, a single-stranded AAV or any combination thereof. 176. The method of any one of embodiments 176-177, wherein the AAV delivery vector is an AAV 2/5 vector, an AAV 2/6 vector, an AAV 2/7 vector, an AAV2/8 vector, or an AAV 2/9 vector.
Embodiment B1. An engineered latent guide RNA that, upon hybridization to a target RNA implicated in a disease or condition, forms a guide-target RNA scaffold comprising a structural feature selected from the group consisting of a bulge, an internal loop, a hairpin, and any combination thereof, wherein the structural feature substantially forms upon hybridization to the target RNA.
Embodiment B2. The engineered latent guide RNA of Embodiment B1, wherein the guide-target RNA scaffold further comprises a mismatch.
Embodiment B3. The engineered latent guide RNA of Embodiment B2, wherein the mismatch is an adenosine/cytosine (A/C) mismatch, wherein the adenosine (A) is present in the target RNA and the cytosine (C) is present in the engineered latent guide RNA.
Embodiment B4. The engineered latent guide RNA of any one of Embodiment B1-Embodiment B3, wherein the guide-target RNA scaffold comprises a wobble base pair.
Embodiment B5. The engineered latent guide RNA of any one of Embodiment B1-Embodiment B3, wherein the guide-target RNA scaffold is a substrate for an RNA editing entity that chemically modifies a base of a nucleotide in the target RNA.
Embodiment B6. The engineered latent guide RNA of any one of Embodiment B3-Embodiment B5, wherein the RNA editing entity chemically modifies the adenosine in the target RNA to an inosine.
Embodiment B7. The engineered latent guide RNA of any one of Embodiment B1-Embodiment B6, wherein the guide-target RNA scaffold comprises a structured motif comprising two or more structural features selected from the group consisting of a bulge, an internal loop, a hairpin, and any combination thereof.
Embodiment B8. The engineered latent guide RNA of any one of Embodiment B1-Embodiment B6, wherein the guide-target RNA scaffold comprises at least two, three, four, five, six, seven, eight, nine, or 10 structural features selected from the group consisting of a bulge, an internal loop, a hairpin, and any combination thereof.
Embodiment B9. The engineered latent guide RNA of any one of Embodiment B1-Embodiment B8, wherein the structural feature is a bulge.
Embodiment B10. The engineered latent guide RNA of Embodiment B9, wherein the bulge is an asymmetric bulge.
Embodiment B11. The engineered latent guide RNA of Embodiment B9, wherein the bulge is a symmetric bulge.
Embodiment B12. The engineered latent guide RNA of any one of Embodiment B9-Embodiment B11, wherein the bulge comprises from 1 to 4 nucleotides of the engineered latent guide RNA and from 0 to 4 nucleotides of the target RNA.
Embodiment B13. The engineered latent guide RNA of any one of Embodiment B9-Embodiment B11, wherein the bulge comprises from 0 to 4 nucleotides of the engineered latent guide RNA and from 1 to 4 nucleotides of the target RNA.
Embodiment B14. The engineered latent guide RNA of Embodiment B10, wherein the asymmetric bulge is an X1/X2 asymmetric bulge, wherein X1 is the number of nucleotides of the target RNA in the asymmetric bulge and X2 is the number of nucleotides of the engineered latent guide RNA in the asymmetric bulge, wherein the X1/X2 asymmetric bulge is a 0/1 asymmetric bulge, a 1/0 asymmetric bulge, a 0/2 asymmetric bulge, a 2/0 asymmetric bulge, a 0/3 asymmetric bulge, a 3/0 asymmetric bulge, a 0/4 asymmetric bulge, a 4/0 asymmetric bulge, a 1/2 asymmetric bulge, a 2/1 asymmetric bulge, a 1/3 asymmetric bulge, a 3/1 asymmetric bulge, a 1/4 asymmetric bulge, a 4/1 asymmetric bulge, a 2/3 asymmetric bulge, a 3/2 asymmetric bulge, a 2/4 asymmetric bulge, a 4/2 asymmetric bulge, a 3/4 asymmetric bulge, or a 4/3 asymmetric bulge.
Embodiment B15. The engineered latent guide RNA of Embodiment B11, wherein the symmetric bulge is an X1/X2 symmetric bulge, wherein X1 is the number of nucleotides of the target RNA in the symmetric bulge and X2 is the number of nucleotides of the engineered latent guide RNA in the symmetric bulge, and wherein the X1/X2 symmetric bulge a 2/2 symmetric bulge, a 3/3 symmetric bulge, or a 4/4 symmetric bulge.
Embodiment B16. The engineered latent guide RNA of any one of Embodiment B1-Embodiment B8, wherein the structural feature comprises an internal loop.
Embodiment B17. The engineered latent guide RNA of Embodiment B16, wherein the internal loop comprises an asymmetric internal loop.
Embodiment B18. The engineered latent guide RNA of Embodiment B16, wherein the internal loop comprises a symmetric internal loop.
Embodiment B19. The engineered latent guide RNA of Embodiment B17, wherein the asymmetric internal loop is an X1/X2 asymmetric internal loop, wherein X1 is the number of nucleotides of the target RNA in the asymmetric internal loop and X2 is the number of nucleotides of the engineered latent guide RNA in the asymmetric internal loop, and wherein the X1/X2 asymmetric internal loop is a 5/6 asymmetric internal loop, a 6/5 asymmetric internal loop, a 5/7 asymmetric internal loop, a 7/5 asymmetric internal loop, a 5/8 asymmetric internal loop, a 8/5 asymmetric internal loop, a 5/9 asymmetric internal loop, a 9/5 asymmetric internal loop, a 5/10 asymmetric internal loop, a 10/5 asymmetric internal loop, a 6/7 asymmetric internal loop, a 7/6 asymmetric internal loop, a 6/8 asymmetric internal loop, a 8/6 asymmetric internal loop, a 6/9 asymmetric internal loop, a 9/6 asymmetric internal loop, a 6/10 asymmetric internal loop, a 10/6 asymmetric internal loop, a 7/8 asymmetric internal loop, a 8/7 asymmetric internal loop, a 7/9 asymmetric internal loop, a 9/7 asymmetric internal loop, a 7/10 asymmetric internal loop, a 10/7 asymmetric internal loop, a 8/9 asymmetric internal loop, a 9/8 asymmetric internal loop, a 8/10 asymmetric internal loop, a 10/8 asymmetric internal loop, or a 9/10 asymmetric internal loop, or a 10/9 asymmetric internal loop.
Embodiment B20. The engineered latent guide RNA of Embodiment B18, wherein the symmetric internal loop is an X1/X2 symmetric internal loop, wherein X1 is the number of nucleotides of the target RNA in the symmetric internal loop and X2 is the number of nucleotides of the engineered latent guide RNA in the symmetric internal loop, and wherein the X1/X2 symmetric internal loop is a 5/5 symmetric internal loop, a 6/6 symmetric internal loop, a 7/7 symmetric internal loop, a 8/8 symmetric internal loop, a 9/9 symmetric internal loop, a 10/10 symmetric internal loop, a 12/12 symmetric internal loop, a 15/15 symmetric internal loop, or a 20/20 symmetric internal loop.
Embodiment B21. The engineered latent guide RNA of any one of Embodiment B16-Embodiment B20, wherein the internal loop is formed by at least 5 nucleotides on either the engineered latent guide RNA or the target RNA.
Embodiment B22. The engineered latent guide RNA of any one of Embodiment B16-Embodiment B21, wherein the internal loop is formed by from 5 to 1000 nucleotides of either the engineered latent guide RNA or the target RNA.
Embodiment B23. The engineered latent guide RNA of any one of Embodiment B16-Embodiment B22, wherein the internal loop is formed by from 5 to 50 nucleotides of either the engineered latent guide RNA or the target RNA.
Embodiment B24. The engineered latent guide RNA of any one of Embodiment B16-Embodiment B23, wherein the internal loop is formed by from 5 to 20 nucleotides of either the engineered latent guide RNA or the target RNA.
Embodiment B25. The engineered latent guide RNA of any one of Embodiment B1-Embodiment B8, wherein the structural feature comprises a hairpin.
Embodiment B26. The engineered latent guide RNA of Embodiment B25, wherein the hairpin comprises a non-recruitment hairpin.
Embodiment B27. The engineered latent guide RNA of Embodiment B25 or Embodiment B26, wherein a loop portion of the hairpin comprises from about 3 to about 15 nucleotides in length.
Embodiment B28. The engineered latent guide RNA of any one of Embodiment B1-Embodiment B27, wherein the engineered latent guide RNA further comprises at least two additional structural features that comprise at least two mismatches.
Embodiment B29. The engineered latent guide RNA of Embodiment B28, wherein at least one of the at least two mismatches is a G/G mismatch.
Embodiment B30. The engineered latent guide RNA of any one of Embodiment B1-Embodiment B29, wherein the engineered latent guide RNA further comprises an additional structural feature that comprises a wobble base pair.
Embodiment B31. The engineered latent guide RNA of Embodiment B30, wherein the wobble base pair comprises a guanine paired with a uracil.
Embodiment B32. The engineered latent guide RNA of Embodiment B6-Embodiment B31, wherein the target RNA comprises a 5′ guanosine adjacent to the adenosine in the target RNA that is chemically modified to an inosine by the RNA editing entity.
Embodiment B33. The engineered latent guide RNA of Embodiment B32, wherein the engineered latent guide RNA comprises a 5′ guanosine adjacent to the cytosine of the A/C mismatch.
Embodiment B34. The engineered latent guide RNA of any one of Embodiment B1-Embodiment B33, wherein the guide-target RNA scaffold mimics a naturally occurring substrate of an ADAR enzyme.
Embodiment B35. The engineered latent guide RNA of any one of Embodiment B1-Embodiment B34, wherein the guide-target RNA scaffold mimics a naturally occurring substrate to a Drosophila ADAR enzyme.
Embodiment B36. The engineered latent guide RNA of any one of Embodiment B5-Embodiment B35, wherein the RNA editing entity is:
(a) an adenosine deaminase acting on RNA (ADAR);
(b) a catalytically active fragment of (a);
(c) a fusion polypeptide comprising (a) or (b); or
(d) any combination of these.
Embodiment B37. The engineered latent guide RNA of any one of Embodiment B5-Embodiment B36, wherein the RNA editing entity is endogenous to a cell.
Embodiment B38. The engineered latent guide RNA of any one of Embodiment B5-Embodiment B37, wherein the RNA editing entity comprises an ADAR.
Embodiment B39. The engineered latent guide RNA of Embodiment B38, wherein the ADAR comprises human ADAR (hADAR).
Embodiment B40. The engineered latent guide RNA of Embodiment B38, wherein the ADAR comprises ADAR1, ADAR2, ADAR3, or any combination thereof.
Embodiment B41. The engineered latent guide RNA of Embodiment B38, wherein the ADAR1 comprises ADAR1p110, ADAR1p150, or a combination thereof.
Embodiment B42. The engineered latent guide RNA of any one of Embodiment B1-Embodiment B41, wherein the engineered latent guide RNA comprises a modified RNA base, an unmodified RNA base, or a combination thereof.
Embodiment B43. The engineered latent guide RNA of any one Embodiment B1-Embodiment B42, wherein the target RNA is an mRNA molecule.
Embodiment B44. The engineered latent guide RNA of any one of Embodiment B1-Embodiment B42, wherein the target RNA is a pre-mRNA molecule.
Embodiment B45. The engineered latent guide RNA of any one of Embodiment B1-Embodiment B44, wherein the target RNA is APP, ABCA4, SERPINA1, HEXA, LRRK2, CFTR, SNCA, MAPT, or LIPA, a fragment any of these, or any combination thereof.
Embodiment B46. The engineered latent guide RNA of any one of 1-Embodiment B44, wherein the target RNA encodes amyloid precursor polypeptide, ATP-binding cassette, sub-family A, member 4 (ABCA4) polypeptide, alpha-1 antitrypsin (AAT) polypeptide, hexosaminidase A enzyme, leucine-rich repeat kinase 2 (LRRK2) polypeptide, CFTR polypeptide, alpha synuclein polypeptide, Tau polypeptide, or lysosomal acid lipase polypeptide.
Embodiment B47. The engineered latent guide RNA of Embodiment B45 or Embodiment B46, wherein the target RNA encodes ABCA4 polypeptide.
Embodiment B48. The engineered latent guide RNA of Embodiment B47, wherein the target RNA comprises a G to A substitution at position 5882, 6320, or 5714, relative to a wildtype ABCA4 gene sequence of accession number NC_000001.11:c94121149-93992837.
Embodiment B49. The engineered latent guide RNA of Embodiment B47 or Embodiment B48, wherein the guide-target RNA scaffold comprises one or more structural features selected from TABLE 7, TABLE, 9, TABLE 10, TABLE 11, TABLE 18, or TABLE 19.
Embodiment B50. The engineered latent guide RNA of any one of Embodiment B47-Embodiment B49, wherein the guide-target RNA scaffold comprises a structural features selected from the group consisting of: (i) one or more X1/X2 bulges, wherein X1 is the number of nucleotides of the target RNA in the bulge and X2 is the number of nucleotides of the engineered latent guide RNA in the bulge, and wherein the one or more bulges is a 2/1 asymmetric bulge, a 1/0 asymmetric bulge, a 2/2 symmetric bulge, a 3/3 symmetric bulge, or a 4/4 symmetric bulge; (ii) an X1/X2 internal loop, wherein X1 is the number of nucleotides of the target RNA in the internal loop and X2 is the number of nucleotides of the engineered latent guide RNA in the internal loop, and wherein the internal loop is a 5/5 symmetric loop (iii) one or more mismatches, wherein the one or more mismatches is a G/G mismatch, an A/C mismatch, or a G/A mismatch, (iv) a G/U wobble base pair or a U/G wobble base pair, and (v) any combination thereof.
Embodiment B51. The engineered latent guide RNA of Embodiment B50, wherein the guide-target RNA scaffold comprises a 2/1 asymmetric bulge, a 1/0 asymmetric bulge, a G/G mismatch, an A/C mismatch, and a 3/3 symmetric bulge.
Embodiment B52. The engineered latent guide RNA of any one of Embodiment B47-Embodiment B51, wherein the engineered latent guide RNA has a length of from 80 to 175 nucleotides.
Embodiment B53. The engineered latent guide RNA of any one of Embodiment B47-Embodiment B52, wherein the engineered latent guide RNA comprises a polynucleotide having at least 80%, at least 85%, at least 90%, at least 95%, at least 97%, at least 99%, or 100% sequence identity to SEQ ID NO: 21, SEQ ID NO: 29, SEQ ID NO: 11, SEQ ID NO: 22, SEQ ID NO: 30, SEQ ID NO: 12, SEQ ID NO: 339-SEQ ID NO: 341, or SEQ ID NO: 292-SEQ ID NO: 296.
Embodiment B54. The engineered latent guide RNA of Embodiment B47-Embodiment B52, wherein the engineered latent guide RNA comprises a polynucleotide at least 80%, at least 85%, at least 90%, at least 95%, at least 97%, at least 99%, or 100% sequence identity to any one of SEQ ID NO: 11-34, 58, 218-289, 291-296, or 328-343.
Embodiment B55. The engineered latent guide RNA of Embodiment B45 or Embodiment B46, wherein the target RNA encodes LRRK2 polypeptide.
Embodiment B56. The engineered latent guide RNA of Embodiment B55, wherein the LRRK2 polypeptide comprises a mutation selected from the group consisting of: E10L, A30P, S52F, E46K, A53T, L119P, A211V, C228S, E334K, N363S, V366M, A419V, R506Q, N544E, N551K, A716V, M712V, 1723V, P755L, R793M, 1810V, K871E, Q923H, Q930R, R1067Q, 51096C, Q1111H, I1122V, A1151T, L1165P, I1192V, H1216R, 51228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, or Q2490NfsX3.
Embodiment B57. The engineered latent guide RNA of Embodiment B55 or Embodiment B56, wherein the guide-target RNA scaffold comprises one or more structural features selected from TABLE 12, TABLE 15, TABLE 25, TABLE 26, TABLE 27, TABLE 17, or TABLE 20.
Embodiment B58. The engineered latent guide RNA of any one of Embodiment B55-Embodiment B57, wherein the guide-target RNA scaffold comprises one or more structural features selected from the group consisting of: (i) one or more X1/X2 bulges, wherein X1 is the number of nucleotides of the target RNA in the bulge and X2 is the number of nucleotides of the engineered latent guide RNA in the bulge, and wherein the one or more bulges is a 0/1 asymmetric bulge, a 2/2 symmetric bulge, a 3/3 symmetric bulge, or a 4/4 symmetric bulge; (ii) one or more X1/X2 internal loops, wherein X1 is the number of nucleotides of the target RNA in the internal loop and X2 is the number of nucleotides of the engineered latent guide RNA in the internal loop, and wherein the one or more internal loops is a 5/0 asymmetric internal loop, a 5/4 asymmetric internal loop, a 5/5 symmetric internal loop, a 6/6 symmetric internal loop, a 7/7 symmetric internal loop, or a 10/10 symmetric internal loop; (iii) one or more mismatches, wherein the one or more mismatches is an A/C mismatch, an A/G mismatch, a C/U mismatch, a G/A mismatch, or a C/C mismatch, (iv) a G/U wobble base pair or a U/G wobble base pair, and (v) any combination thereof.
Embodiment B59. The engineered latent guide RNA of Embodiment B58, wherein the guide-target RNA scaffold comprises a 6/6 symmetrical internal loop, an A/C mismatch, an A/G mismatch, and a C/U mismatch.
Embodiment B60. The engineered latent guide RNA of any one of Embodiment B55-Embodiment B59, wherein the engineered latent guide RNA has a length of from 80 to 175 nucleotides.
Embodiment B61. The engineered latent guide RNA of any one of Embodiment B55-Embodiment B60, wherein the engineered latent guide RNA comprises a polynucleotide having at least 80%, at least 85%, at least 90%, at least 95%, at least 97%, at least 99%, or 100% sequence identity to SEQ ID NO: 30, SEQ ID NO: 344, or SEQ ID NO: 345.
Embodiment B62. The engineered latent guide RNA of Embodiment B55-Embodiment B60, wherein the engineered latent guide RNA comprises a polynucleotide having at least 80%, at least 85%, at least 90%, at least 95%, at least 97%, at least 99%, or 100% sequence identity to any one of SEQ ID NO: 35-42, 46-52, 111-207, or 344-345.
Embodiment B63. The engineered latent guide RNA of Embodiment B45 or Embodiment B46, wherein the target RNA encodes SNCA polypeptide.
Embodiment B64. The engineered latent guide RNA of Embodiment B63, wherein the engineered latent guide RNA hybridizes to a sequence of the target RNA selected from the group consisting of: a 5′ untranslated region (UTR), a 3′ UTR, and a translation initiation site of an SNCA gene.
Embodiment B65. The engineered latent guide RNA of Embodiment B63 or Embodiment B64, wherein the guide-target RNA scaffold comprises one or more structural features selected from TABLE 21, TABLE 23, or TABLE 28.
Embodiment B66. The engineered latent guide RNA of any one of Embodiment B63-Embodiment B65, wherein the guide-target RNA scaffold comprises one or more structural features selected from the group consisting of: (i) an X1/X2 bulge, wherein X1 is the number of nucleotides of the target RNA in the bulge and X2 is the number of nucleotides of the engineered latent guide RNA in the bulge, and wherein the bulge is a 4/4 symmetric bulge; (ii) one or more X1/X2 internal loops, wherein X1 is the number of nucleotides of the target RNA in the internal loop and X2 is the number of nucleotides of the engineered latent guide RNA in the internal loop, and wherein the one or more internal loop is a 5/5 symmetric loop, an 8/8 symmetric loop, or a 49/4 asymmetric loop; (iii) one or more mismatches, wherein the one or more mismatches is an A/C mismatch, a G/G mismatch, a G/A mismatch, a U/C mismatch, or an A/A mismatch, (iv) any combination thereof.
Embodiment B67. The engineered latent guide RNA of Embodiment B66, wherein the engineered latent guide RNA has a length of from 80 to 175 nucleotides.
Embodiment B68. The engineered latent guide RNA of any one of Embodiment B63-Embodiment B66, wherein the engineered latent guide RNA comprises a polynucleotide having at least 80%, at least 85%, at least 90%, at least 95%, at least 97%, at least 99%, or 100% sequence identity to any one of SEQ ID NO: 59-101, 104-108, and 208-217.
Embodiment B69. The engineered latent guide RNA of Embodiment B45 or Embodiment B46, wherein the target RNA encodes SERPINA1.
Embodiment B70. The engineered latent guide RNA of Embodiment B69, wherein the target RNA comprises a G to A substitution at position 9989, relative to a wildtype SERPINA1 gene sequence of accession number NC_000014.9:c94390654-94376747.
Embodiment B71. The engineered latent guide RNA of Embodiment B69 or Embodiment B70, wherein the guide-target RNA scaffold comprises one or more structural features selected from TABLE 5, TABLE 29, TABLE 30, TABLE 31, TABLE 32, TABLE 33, TABLE 34, TABLE 35, or TABLE 36.
Embodiment B72. The engineered latent guide RNA of any one of Embodiment B69-Embodiment B71, wherein the guide-target RNA scaffold comprises one or more structural features selected from the group consisting of: (i) one or more X1/X2 bulges, wherein X1 is the number of nucleotides of the target RNA in the bulge and X2 is the number of nucleotides of the engineered latent guide RNA in the bulge, and wherein the bulge is a 0/2 asymmetric bulge, a 0/3 asymmetric bulge, a 1/0 asymmetric bulge, a 2/0 asymmetric bulge, a 2/2 symmetric bulge, a 3/0 asymmetric bulge, a 2/2 symmetric bulge, or a 3/3 symmetric bulge; (ii) an X1/X2 internal loop, wherein X1 is the number of nucleotides of the target RNA in the internal loop and X2 is the number of nucleotides of the engineered latent guide RNA in the internal loop, and wherein the internal loop is a 5/5 symmetric internal loop; (iii) one or more mismatches, wherein the one or more mismatches is an A/C mismatch, an A/A mismatch, and a G/A mismatch, (iv) a G/U wobble base pair, or a U/G wobble base pair; and (v) any combination thereof.
Embodiment B73. The engineered latent guide RNA of Embodiment B72, wherein the engineered latent guide RNA has a length of from 80 to 175 nucleotides.
Embodiment B74. The engineered latent guide RNA of any one of Embodiment B69-Embodiment B73, wherein the engineered latent guide RNA comprises a polynucleotide having at least 80%, at least 85%, at least 90%, at least 95%, at least 97%, at least 99%, or 100% sequence identity to any one of SEQ ID NO: 6-10, 102-103 or 297-327.
Embodiment B75. The engineered latent guide RNA of 1-Embodiment B74, wherein the base of the nucleotide of the target RNA that is modified by the RNA editing entity is comprised in a point mutation of the target RNA.
Embodiment B76. The engineered latent guide RNA of Embodiment B75, wherein the point mutation comprises a missense mutation.
Embodiment B77. The engineered latent guide RNA of Embodiment B75, wherein the point mutation is a nonsense mutation.
Embodiment B78. The engineered latent guide RNA of Embodiment B77, wherein the nonsense mutation is a premature UAA stop codon.
Embodiment B79. The engineered latent guide RNA of any one of 1-Embodiment B78, wherein the structural feature increases selectivity of editing a target adenosine in the target RNA relative to an otherwise comparable guide RNA lacking the structural feature.
Embodiment B80. The engineered latent guide RNA of any one of 1-Embodiment B79, wherein the structural feature decreases an amount of RNA editing of local off-target adenosines within 200, within 100, within 50, within 25, within 10, within 5, within 2, or 1 within 1 nucleotides 5′ or 3′ of a target adenosine in the target RNA by the RNA editing entity, relative to an otherwise comparable guide RNA lacking the structural feature.
Embodiment B81. An engineered RNA comprising:
(a) the engineered latent guide RNA of any one of 1-Embodiment B80,
(b) a U7 snRNA hairpin sequence, a SmOPT sequence, or a combination thereof.
Embodiment B82. The engineered RNA of Embodiment B81, wherein the U7 hairpin has a sequence of TAGGCTTTCTGGCTTTTTACCGGAAAGCCCCT (SEQ ID NO: 389) or CAGGTTTTCTGACTTCGGTCGGAAAACCCCT (SEQ ID NO: 394).
Embodiment B83. The engineered RNA of Embodiment B81, wherein the SmOPT sequence has a sequence of AATTTTTGGAG (SEQ ID NO: 390).
Embodiment B84. A polynucleotide encoding the engineered latent guide RNA of any one of Embodiment B1-Embodiment B80 or the engineered RNA of any one of Embodiment B81-Embodiment B83.
Embodiment B85. A delivery vector comprising the engineered latent guide RNA of any one of Embodiment B1-Embodiment B80, the engineered RNA of any one of Embodiment B81-Embodiment B83, or the polynucleotide of Embodiment B84.
Embodiment B86. The delivery vector of Embodiment B85, wherein the delivery vector is a viral vector.
Embodiment B87. The delivery vector of Embodiment B86, wherein the viral vector is an adeno-associated viral (AAV) vector or a derivative thereof.
Embodiment B88. The delivery vector of Embodiment B87, wherein the AAV vector is from an adeno-associated virus having a serotype selected from AAV1, AAV2, AAV3, AAV4, AAV5, AAV6, AAV7, AAV8, AAV9, AAV10, AAV 11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68.
Embodiment B89. The delivery vector of Embodiment B87 or Embodiment B88, wherein the AAV vector is a recombinant AAV (rAAV) vector, a hybrid AAV vector, a chimeric AAV vector, a self-complementary AAV (scAAV) vector, a single-stranded AAV or any combination thereof.
Embodiment B90. The delivery vector of any one of Embodiment B87-Embodiment B89, wherein the AAV vector comprises a genome comprising a replication gene and inverted terminal repeats from a first AAV serotype and a capsid protein from a second AAV serotype.
Embodiment B91. The delivery vector of any one of Embodiment B87-Embodiment B90, wherein the AAV vector is an AAV 2/5 vector, an AAV 2/6 vector, an AAV 2/7 vector, an AAV2/8 vector, or an AAV 2/9 vector.
Embodiment B92. The delivery vector of Embodiment B90, wherein the inverted terminal repeats comprise a 5′ inverted terminal repeat, a 3′ inverted terminal repeat, and a mutated inverted terminal repeat.
Embodiment B93. The delivery vector of Embodiment B92, wherein the mutated inverted terminal repeat lacks a terminal resolution site.
Embodiment B94. A pharmaceutical composition comprising:
(a) engineered latent guide RNA of any one of Embodiment B1-Embodiment B80, the engineered RNA of any one of Embodiment B81-Embodiment B83, the polynucleotide of Embodiment B84, or the delivery vector of any one of Embodiment B85-Embodiment B93, and
(b) a pharmaceutically acceptable: excipient, carrier, or diluent.
Embodiment B95. The pharmaceutical composition of Embodiment B94, in unit dose form.
Embodiment B96. The pharmaceutical composition of Embodiment B94 or Embodiment B95, further comprising an additional therapeutic agent.
Embodiment B97. The pharmaceutical composition of Embodiment B96, wherein the additional therapeutic agent comprises an ammonia reducer, a beta blocker, a synthetic hormone, an antibiotic, or an antiviral drug, a vascular endothelial growth factor (VEGF) inhibitor, a stem cell treatment, a vitamin or modified form thereof, or any combination thereof.
Embodiment B98. A method of editing a target RNA in a cell, the method comprising: administering to the cell an effective amount of the engineered latent guide RNA of any one of 1-Embodiment B80, the engineered RNA of any one of Embodiment B81-Embodiment B83, the polynucleotide of Embodiment B84, the delivery vector of any one of Embodiment B85-Embodiment B93, or the pharmaceutical composition of any one of Embodiment B94-Embodiment B97.
Embodiment B99. A method of treating a disease in a subject, the method comprising administering to the subject an effective amount of the engineered latent guide RNA of any one of 1-Embodiment B80, the engineered RNA of any one of Embodiment B81-Embodiment B83, the polynucleotide of Embodiment B84, the delivery vector of any one of Embodiment B85-Embodiment B93, or the pharmaceutical composition of any one of Embodiment B94-Embodiment B97.
Embodiment B100. The method of Embodiment B98, wherein the engineered latent guide RNA is administered as a unit dose.
Embodiment B101. The method of Embodiment B100, wherein the unit dose is an amount sufficient to treat the subject.
Embodiment B102. The method of any one of Embodiment B98-Embodiment B101, wherein the administering is intrathecal, intraocular, intravitreal, retinal, intravenous, intramuscular, intraventricular, intracerebral, intracerebellar, intracerebroventricular, intraperenchymal, subcutaneous, or a combination thereof.
Embodiment B103. The method of any one of Embodiment B98-Embodiment B102, wherein the disease comprises a neurological disease.
Embodiment B104. The method of Embodiment B103, wherein the neurological disease comprises Parkinson's disease, Alzheimer's disease, a Tauopathy, or dementia.
Embodiment B105. The method of Embodiment B103 or Embodiment B104, wherein the neurological disease is associated with elevated levels of SNCA polypeptide, relative to a healthy subject that does not have the neurological disease or condition.
Embodiment B106. The method of Embodiment B105, wherein the engineered latent guide RNA hybridizes to a sequence of a target RNA encoding the SNCA polypeptide selected from the group consisting of: a 5′ untranslated region (UTR), a 3′ UTR, and a translation initiation site of SNCA; wherein hybridization produces a guide-target RNA scaffold that is a substrate for an RNA editing entity that chemically modifies a base of a nucleotide in the sequence of the target RNA, thereby reducing levels of the SNCA polypeptide.
Embodiment B107. The method of Embodiment B106, wherein the engineered latent guide RNA hybridizes to a sequence of a target RNA encoding the translation initiation site of SNCA.
Embodiment B108. The method of any one of Embodiment B105-Embodiment B107, wherein the engineered latent guide RNA comprises a polynucleotide having at least 80%, at least 85%, at least 90%, at least 95%, at least 97%, at least 99%, or 100% sequence identity to any one of SEQ ID NO: 59-101, 104-108, and 208-217.
Embodiment B109. The method of any one of Embodiment B105-Embodiment B108, wherein the engineered latent guide RNA comprises has a percent on-target editing for ADAR2 of at least about 90%.
Embodiment B110. The method of Embodiment B103 or Embodiment B104, wherein the neurological disease is associated with a mutation of an LRRK2 polypeptide encoded by the target RNA, wherein the mutation is selected from the group consisting of: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, 12012T, G2019S, I2020T, T2031S, N2081D, T2141M, R2143H, Y2189C, T2356I, G2385R, V2390M, E2395K, M2397T, L2466H, or Q2490NfsX3.
Embodiment B111. The method of Embodiment B103 or Embodiment B104, wherein the neurological disease is associated with a mutation of an LRRK2 polypeptide encoded by the target RNA, wherein the mutation is a G2019S mutation.
Embodiment B112. The method of any one of Embodiment B110-114, wherein the engineered latent guide RNA comprises a polynucleotide having at least 80%, at least 85%, at least 90%, at least 95%, at least 97%, at least 99%, or 100% sequence identity to any one of SEQ ID NO: 35-42, 46-52, 111-207, or 344-345.
Embodiment B113. The method of any one of Embodiment B110-Embodiment B112, wherein the engineered latent guide RNA comprises has a percent on-target editing for ADAR1 of at least about 60% or a percent on-target editing for ADAR2 of at least about 90%.
Embodiment B114. The method of any one of Embodiment B98-Embodiment B102, wherein the disease comprises a liver disease.
Embodiment B115. The method of Embodiment B114, wherein the liver disease comprises liver cirrhosis.
Embodiment B116. The method of Embodiment B114, wherein the liver disease is alpha-1 antitrypsin (AAT) deficiency.
Embodiment B117. The method of Embodiment B116, wherein the AAT deficiency is associated with a G to A substitution at position 9989 of a wildtype SERPINA1 gene sequence of accession number NC_000014.9:c94390654-94376747.
Embodiment B118. The method of Embodiment B116 or Embodiment B117, wherein the engineered latent wherein the engineered latent guide RNA comprises a polynucleotide having at least 80%, at least 85%, at least 90%, at least 95%, at least 97%, at least 99%, or 100% sequence identity to any one of SEQ ID NO: 6-10, 102-103 or 297-327.
Embodiment B119. The method of any one of Embodiment B116-Embodiment B118, wherein the engineered latent guide RNA comprises has a percent on-target editing for ADAR1 of at least about 60% or a percent on-target editing for ADAR2 of at least about 90%.
Embodiment B120. The method of any one of Embodiment B98-Embodiment B102, wherein the disease is a macular degeneration.
Embodiment B121. The method of Embodiment B120, wherein the macular degeneration is Stargardt Disease.
Embodiment B122. The method of Embodiment B121, wherein the Stargardt disease is associated with a G to A substitution at position 5882, 6320, or 5714 of a wildtype ABCA4 gene sequence of accession number NC_000001.11:c94121149-93992837.
Embodiment B123. The method of Embodiment B122, wherein the Stargardt disease is associated with a G to A substitution at position 5882.
Embodiment B124. The method of Embodiment B121 or Embodiment B122, wherein the engineered latent guide RNA comprises a polynucleotide having at least 80%, at least 85%, at least 90%, at least 95%, at least 97%, at least 99%, or 100% sequence identity to any one of SEQ ID NO: 11-34, 58, 218-289, 291-296, or 328-343.
Embodiment B125. The method of any one of Embodiment B122-Embodiment B124, wherein the engineered latent guide RNA comprises has a percent on-target editing for ADAR1 of at least about 70% or a percent on-target editing for ADAR2 of at least about 80%.
Embodiment B126. The method of any one of Embodiment B98-Embodiment B125, wherein the subject is diagnosed with the disease or the condition.
Embodiment B127. The engineered latent guide RNA of any one of 1-Embodiment B80, the engineered RNA of any one of Embodiment B81-Embodiment B83, the polynucleotide of Embodiment B84, the delivery vector of any one of Embodiment B85-Embodiment B93, or the pharmaceutical composition of any one of Embodiment B94-Embodiment B97, for use as a medicament.
Embodiment B128. The engineered latent guide RNA of any one of 1-Embodiment B80, the engineered RNA of any one of Embodiment B81-Embodiment B83, the polynucleotide of Embodiment B84, the delivery vector of any one of Embodiment B85-Embodiment B93, or the pharmaceutical composition of any one of Embodiment B94-Embodiment B97, for use in treatment of a neurological disease.
Embodiment B129. The engineered latent guide RNA, polynucleotide, delivery vector, or pharmaceutical composition for the use of Embodiment B129, wherein the neurological disease is Parkinson's disease, Alzheimer's disease, a Tauopathy, or dementia.
Embodiment B130. The engineered latent guide RNA of any one of 1-Embodiment B80, the engineered RNA of any one of Embodiment B81-Embodiment B83, the polynucleotide of Embodiment B84, the delivery vector of any one of Embodiment B85-Embodiment B93, or the pharmaceutical composition of any one of Embodiment B94-Embodiment B97, for use in treatment of a liver disease.
Embodiment B131. The engineered latent guide RNA, polynucleotide, delivery vector, or pharmaceutical composition for the use of Embodiment B130, wherein the liver disease comprises liver cirrhosis.
Embodiment B132. The engineered latent guide RNA, polynucleotide, delivery vector, or pharmaceutical composition for the use of Embodiment B130, wherein the liver disease is alpha-1 antitrypsin (AAT) deficiency.
Embodiment B133. The engineered latent guide RNA of any one of 1-Embodiment B80, the engineered RNA of any one of Embodiment B81-Embodiment B83, the polynucleotide of Embodiment B84, the delivery vector of any one of Embodiment B85-Embodiment B93, or the pharmaceutical composition of any one of Embodiment B94-Embodiment B97, for use in treatment of macular degeneration.
Embodiment B134. The engineered latent guide RNA, polynucleotide, delivery vector, or pharmaceutical composition for the use of Embodiment B133, wherein the macular degeneration is Stargardt disease.
Embodiment B135. Use of the engineered latent guide RNA of any one of 1-Embodiment B80, the engineered RNA of any one of Embodiment B81-Embodiment B83, the polynucleotide of Embodiment B84, the delivery vector of any one of Embodiment B85-Embodiment B93, or the pharmaceutical composition of any one of Embodiment B94-Embodiment B97, for the manufacture of a medicament.
Embodiment B136. Use of the engineered latent guide RNA of any one of 1-Embodiment B80, the engineered RNA of any one of Embodiment B81-Embodiment B83, the polynucleotide of Embodiment B84, the delivery vector of any one of Embodiment B85-Embodiment B93, or the pharmaceutical composition of any one of Embodiment B94-Embodiment B97, for the manufacture of a medicament for the treatment of a neurological disease, a liver disease or macular degeneration.
Embodiment B137. An engineered latent guide RNA, wherein upon hybridization to a target RNA, forms a guide-target RNA scaffold comprising an A/C mismatch and a structural feature selected from the group consisting of a bulge, an internal loop, a hairpin, a second mismatch, and any combination thereof, wherein the structural feature increases an amount of RNA editing of the target RNA by the RNA editing entity, relative to an otherwise comparable guide RNA lacking the structural feature. The engineered latent guide RNA of embodiment B1, wherein the structural feature substantially forms upon hybridization to the target RNA.

### EXAMPLES

The following examples are included for illustrative purposes only and are not intended to limit the scope of the present disclosure.

### Example 1

### Example Workflow

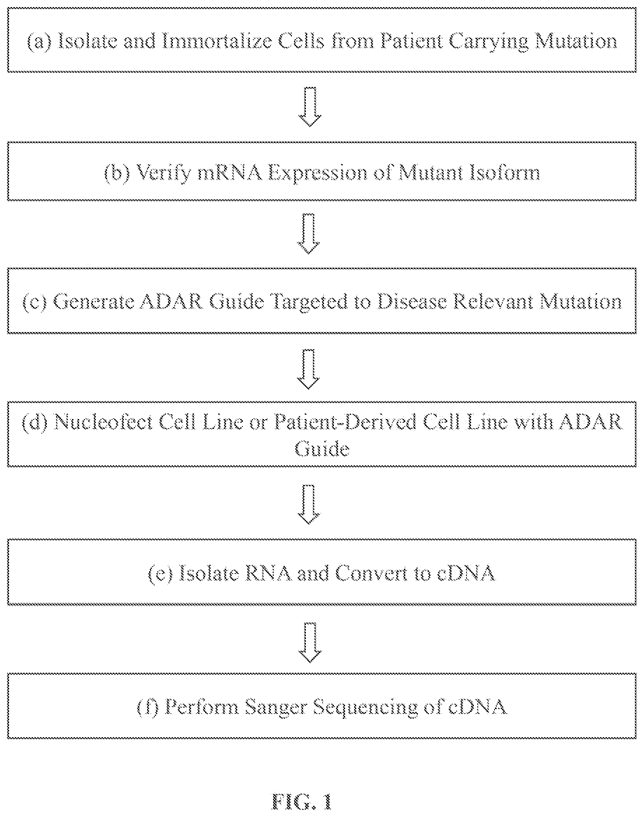

An example of a workflow according to the methods described herein is illustrated in FIG. 1. First mRNA, pre-mRNA, or cells from a patient possessing disease-causing mutations are isolated and immortalized (step a). Second, mRNA expression of the mutation or mutations is verified using DNA or RNA sequencing (e.g., Sanger sequencing) (step b). Third an engineered guide with a targeting region capable of hybridizing to the region of pre-mRNA or mRNA comprising the mutation is recombinantly produced (step c). Fourth, the engineered guide is administered to the patient cells (e.g., via a viral vector). After treatment, to verify editing has occurred, the patient RNA is isolated and converted to cDNA (step e) and then sequenced by Sanger sequencing (step f). In some instances, the mRNA or pre-mRNA does not have a mutation, but includes a target adenosine to be edited to reduce disease pathogenesis. For example, the target mRNA may be APP and an adenosine may be targeted by the guide RNAs for editing by ADAR to reduce cleavage by a secretase enzyme.

### Example 2

### SERPINA1 E342K can be Edited in Fibroblasts from Homozygous Patient

This example describes editing of the SERPINA1 E342K mutation in fibroblasts from a patient carrying the homozygous mutation. Guide RNAs (gRNAs) targeting both mRNA and pre-mRNA were recombinantly produced. gRNAs tested comprised a C at a position opposite the target A in SERPINA1 to be edited, thus yielding a mismatch upon hybridization of the gRNA to the target sequence and formation of a double stranded substrate. gRNAs tested were all linear gRNAs. A summary of the gRNAs tested is provided in TABLE 5 below. The column in TABLE 5 titled “Structural Features” describes structural features in the double stranded RNA substrate formed upon hybridization of the gRNA to the target RNA. For the 1.100.50 gRNA, the internal GluR2 is a pre-formed hairpin (or a recruitment domain) in the gRNA itself. For the engineered guide RNA sequences shown in TABLE 5, lower case letters indicate regions of the guide RNA that target intronic sequence and upper case letters indicate regions of the guide RNA that target exonic sequence.

TABLE 5
Engineered gRNAs against SERPINA1 pre-mRNA

| | | | Structural | Percent |
|---|---|---|---|---|
| | | | Features | On |
| | SEQ | | (target/ | Target |
| Name | ID NO | Guide RNA Sequence | guide) | Editing |
| 0.100.50 | SEQ ID NO: | ATGGGTATGGCCTCTAAAAACATGGCCCCAG | 1 A/C | ~8% |
| Targets | 325 | CAGCTTCAGTCCCTTTCTCGTCGATGGTCAGC | mismatch | |
| pre- | (DNA | ACAGCCTTATGCACGGCctggaggggagagaagcaga | | |
| mRNA | sequence | | | |
| | encoding SEQ | | | |
| | ID NO: 8) | | | |
| 0.100.50 + | SEQ ID NO: | ATGGGTATGGCCTCTAAAAACATGGCCCCAG | 1 A/C | — |
| bulge | 326 | CAGCTTCAGTCATACCTTTCTCGTCGATGGTC | mismatch | |
| Targets | (DNA | AGCATGACAGCCTTATGCACGGCctggaggggagag | 2 3/3 | |
| pre- | sequence for | aagcaga | symmetric | |
| mRNA | SEQ ID NO: | | bulges | |
| | 10) | | | |
| 1.100.50 | SEQ ID NO: | ATGGGTATGGCCTCTAAAAACATGGCCCCAG | 1 A/C | — |
| (internal | 327 | CAGCTTCAGTCCCTTTCTCGTCGATGGTCCAC | mismatch | |
| GluR2) | | CCTATGATATTGTTGTAAATCGTATAACAA | 1 Internal | |
| Targets | | TATGATAAGGTGAGCACAGCCTTATGCACGG | recruiting | |
| pre- | | Cctggaggggagagaagcaga | hairpin | |
| mRNA | | | | |
| 0.100.50 | SEQ ID NO: | ATGGGTATGGCCTCTAAAAACATGGCCCCAG | 1 A/C | ~5% |
| Targets | 391 | CAGCTTCAGTCCCTTTCTCGTCGATGGTCAGC | mismatch | |
| mRNA | (DNA | ACAGCCTTATGCACGGCCTTGGAGAGCTTCAG | | |
| | sequence for | GGGTG | | |
| | SEQ ID NO: | | | |
| | 6) | | | |
| 0.100.50 + | SEQ ID NO: 7 | ATGGGTATGGCCTCTAAAAACATGGCCCCAG | 1 A/C | — |
| bulge | | CAGCTTCAGTCATACCTTTCTCGTCGATGGTC | mismatch | |
| Targets | | AGCATGACAGCCTTATGCACGGCCTTGGAGA | 2 3/3 | |
| mRNA | | GCTTCAGGGGTG | symmetrical | |
| | | | bulges | |
| 1.100.50 | SEQ ID NO: | ATGGGTATGGCCTCTAAAAACATGGCCCCAG | 1 A/C | — |
| (internal | 395 | CAGCTTCAGTCCCTTTCTCGTCGATGGTCCAC | mismatch | |
| GluR2) | | CCTATGATATTGTTGTAAATCGTATAACAA | 1 Internal | |
| Targets | | TATGATAAGGTGAGCACAGCCTTATGCACGG | recruiting | |
| mRNA | | CCTTGGAGAGCTTCAGGGGTG | hairpin | |

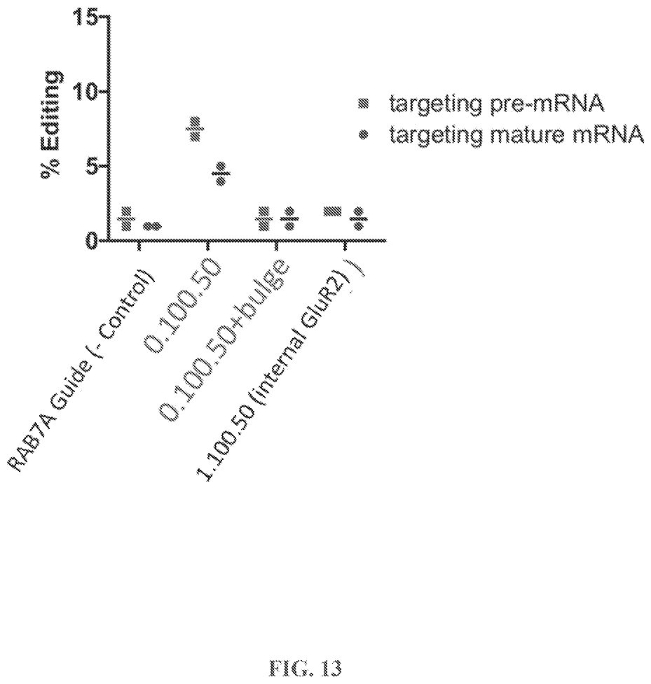

Immortalized cells (fibroblasts) from patients carrying the E342K mutation were grown in culture. The mRNA expression of the mutant isoform of the alpha-1 antitrypsin (AAT) protein from the mutated SERPINA1 gene in the patient was verified using RT-PCR or a suitable antibody that can recognize the mutated isoform. An engineered gRNA against Rab7a (“control gRNA”; negative control), and the engineered gRNAs against SERPINA1 were nucleofected in the fibroblasts. 2×10\hat{ }5 cells were used per transfection and cells were transfected with 60 pmoles of the engineered gRNAs. cDNA synthesized from the isolated RNA was PCR amplified, followed by Sanger sequencing. Percent editing was quantified using the quantification software. FIG. 13 shows the percent editing achieved by each of the gRNAs. In this experiment, gRNAs with a single A/C mismatch and lacking a recruitment domain provided the highest levels of on-target editing.

### Example 3

### Effect of Guide RNA Length and A/C Mismatch Placement in ABCA4 Targeting Engineered Guide RNAs

This example describes the effect on ADAR-mediated RNA editing from changing guide RNA length and A/C mismatch placement in ABCA4 targeting engineered guide RNAs of the present disclosure. Fold change luciferase assays in a broken luciferase screen were performed to assess the impact of altering guide length and mismatch placement on RNA editing of ABCA4. A faux ABCA4 mini-gene carrying ABCA4 mutations of interested were introduced into cells. To generate a faux ABCA4 mini-gene, five nucleotides of the original transcript were modified. These changes are summarized in TABLE 6 below. The GAA to TAG mutation introduced a premature stop codon and the A within the TAG sequence in the target RNA was targeted for RNA editing using the engineered guide RNAs disclosed herein.

TABLE 6
Faux ABCA4 Mutations

| Mutation | Exon | Distance from target A | Rationale |
|---|---|---|---|
| GAA −> TAG | 42 | +1, −1 | Creates a stop codon |
| A −> G | 41 | 80 | No TTTT in guide |
| A −> G | 43 | −52 | No TTTT in guide |
| A −> G | 44 | −154 | No TTTT in guide |

Guides of various lengths and having various mismatch placements were recombinantly produced. gRNAs tested were all linear gRNAs. A summary of the gRNAs tested is provided in TABLE 7 below. The column in TABLE 7 titled “Structural Features” describes structural features in the double stranded RNA substrate formed upon hybridization of the gRNA to the target RNA.

TABLE 7
Engineered gRNAs against ABCA4

| | | | | Fold |
|---|---|---|---|---|
| | | | | Increase |
| | | | Structural | in RNA |
| Name | SEQ ID NO | Guide RNA Sequence | Features | Editing |
| 150.75 | SEQ ID NO: | TGAGCATCTTGAATGTGGTTGTCTTGCCG | 1 A/C mismatch | 2.93 |
| | 328 | GCACCATTCACTCCCAGGAGGCCAAAGCA | | |
| | | CTCTCCAGGGCGAACCCAGACACACAGCC | | |
| | | TGTCCACTGCTGGGCTGGAGGTGCCTGGA | | |
| | | TAAATCTTGGTTAGTTCATGTAGCCTTAA | | |
| | | GATGT | | |
| 100.91 | SEQ ID NO: | ACTGTGGTGTCCCCAGTGAGCATCTTGAA | 1 A/C mismatch | 2.44 |
| | 329 | TGTGGTTGTCTTGCCGGCACCATTCACTCC | | |
| | | CAGGAGGCCAAAGCACTCTCCAGGGCGA | | |
| | | ACCCAGACACACA | | |
| 150.136 | SEQ ID NO: | TTGGTTAAAATACTCTTGCCTGCTACGGT | 1 A/C mismatch | 2.17 |
| | 330 | GGCATCCCCTGAGGTCACTGTGGTGTCCC | | |
| | | CAGTGAGCATCTTGAATGTGGTTGTCTTG | | |
| | | CCGGCACCATTCACTCCCAGGAGGCCAAA | | |
| | | GCACTCTCCAGGGCGAACCCAGACACACA | | |
| | | GCCTG | | |
| 200.100 | SEQ ID NO: | CCTGAGGTCACTGTGGTGTCCCCAGTGAG | 1 A/C mismatch | 2.14 |
| | 331 | CATCTTGAATGTGGTTGTCTTGCCGGCAC | | |
| | | CATTCACTCCCAGGAGGCCAAAGCACTCT | | |
| | | CCAGGGCGAACCCAGACACACAGCCTGTC | | |
| | | CACTGCTGGGCTGGAGGTGCCTGGATAAA | | |
| | | TCTTGGTTAGTTCATGTAGCCTTAAGATGT | | |
| | | CAGTCTTATTTCCACCAGTAATAAT | | |
| 100.50 | SEQ ID NO: | TGCCGGCACCATTCACTCCCAGGAGGCCA | 1 A/C mismatch | 2.13 |
| | 332 | AAGCACTCTCCAGGGCGAACCCAGACAC | | |
| | | ACAGCCTGTCCACTGCTGGGCTGGAGGTG | | |
| | | CCTGGATAAATCTT | | |

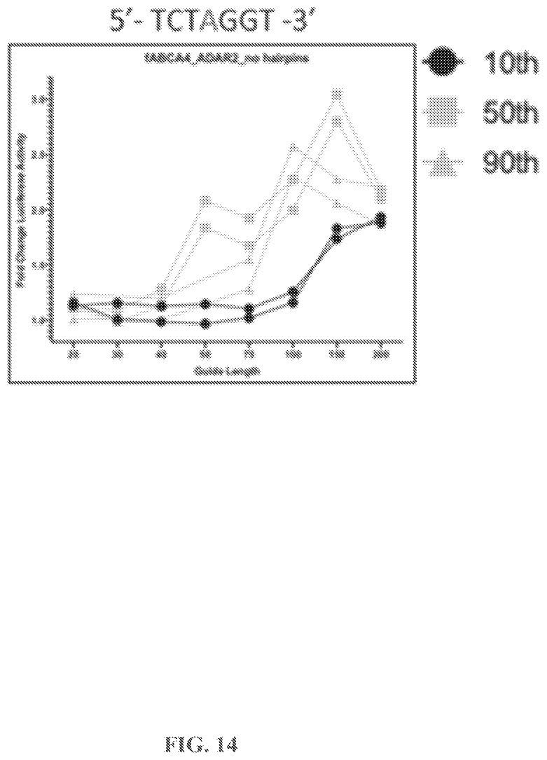

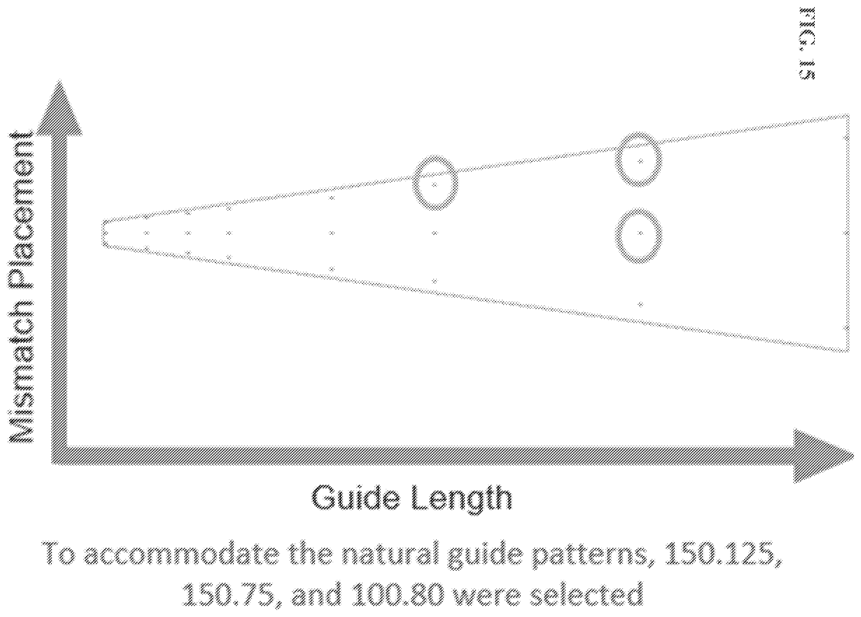

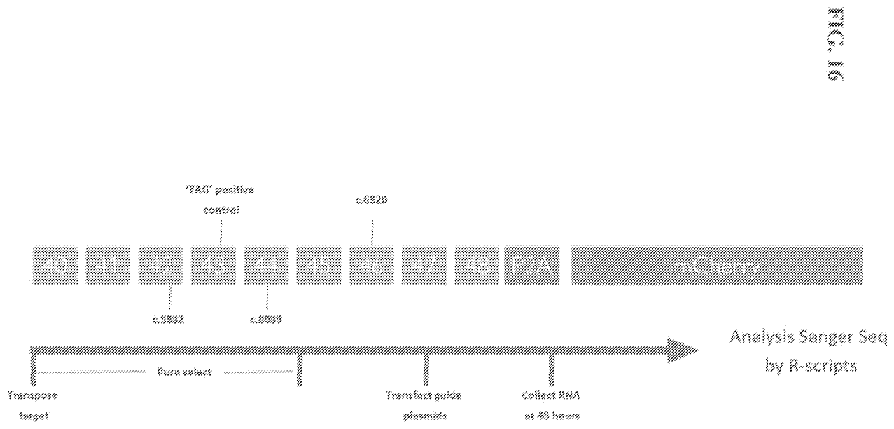

As shown in FIG. 14, the 150.75 guide exhibited the highest fold change in luciferase. The 10^{th}, 50^{th}, and 90^{th} lines refers to the percentile of the A/C mismatch as it's expressed from 5′ to 3′ and the x-axis indicates the length of each guide RNA. The lengths and mismatch placements in the various guides are detailed in the names recited in TABLE 7. For example, guide “150.75” refers to a guide 150 nucleotides in length, wherein the C that forms the A/C mismatch in the double stranded substrate formed upon hybridization of the engineered guide RNA and the target RNA is located at nucleotide 75, plus or minus 2 nucleotides, of the engineered guide RNA strand counting from the 5′ to 3′ direction. The same nomenclature was used for the remainder of the engineered guide RNAs in TABLE 7.

Engineered guides 150.125, 150.75, and 100.80 were selected to move forward into the experiments detailed below.

### Example 4

### Correction of Mutations in ABCA4 with Engineered Guide RNAs

This example describes engineered guide RNAs of the present disclosure designed to target mutations in ABCA4 RNA for correction. Stargardt disease may be caused by loss-of-function genetic mutations in the ABCA4 gene. The most common mutations present in Stargardt's disease patients are detailed below in TABLE 8, with mutations amenable to correction by ADAR listed in bold text. The most common missense mutations in Stargardt disease include G>A mutation (e.g., the c.5882 G>A mutation), which can be corrected by ADAR. However, ADARs may be generally disinclined to deaminate adenosines with an upstream 5′G, as is the case with targeting the c.5882G>A mutation.

Experiments described herein were conducted to assess the ability of engineered guide RNAs that, upon hybridization to a target ABCA4 sequence, form structural features in the resulting double stranded RNA substrate, as disclosed herein, to correct the c.5882G>A mutations expressed in ABCA4 miniaturized genes (minigenes). Without being bound by any theory, the steric hindrance produced by the structural features may have improved ADAR deamination of adenosines with a 5′G, as compared to ADAR deamination of adenosines facilitated by engineered guide RNAs that do not form said structural features upon hybridization to the target ABCA4 RNA.

TABLE 8
Stargardt Disease Associated Mutations

| | Allele Frequency in Total |
|---|---|
| | Cohort with Multiple |
| Nucleotide change, Amino acid change | Likely Pathogenic Variants |
| c.5882G > A, p. Gly1961Glu | 15.05% |
| c.2588G > C, p. Gly863Ala | 7.17% |
| c.5461 − 10T > C, splice site alteration | 4.84% |
| c.4139C > T, p. Pro1380Leu | 3.94 |
| c. 1622T > C, p. Leu541Pro | 2.69 |
| c.5714 + 5G > A, splice site alteration | 2.33 |
| c.3322C > T, p. Arg1108Cys | 2.33 |
| c.6079C > T, p. Leu2027Phe | 2.33 |
| c.6320G > A, p. Arg2107His | 1.61 |
| c.6089G > A, p. Arg2030Gln | 1.61 |

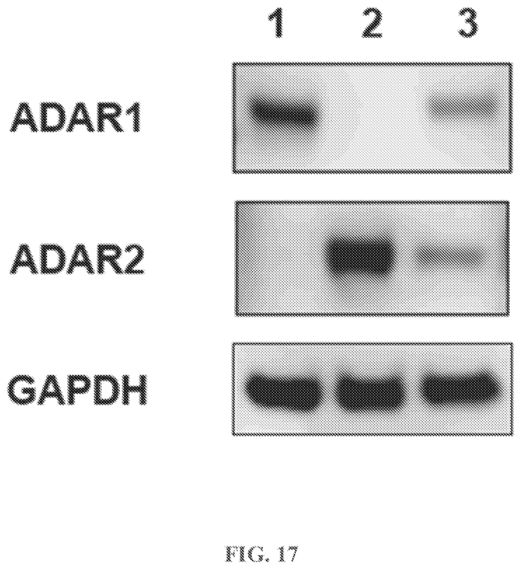

Wild type HEK293 cells expressing a minigene containing exons 40-48, in frame, of ABCA4 with a downstream P2A-mCherry were produced. As illustrated in FIG. 16, exon 42 of the minigene expresses the c.5882G>A mutation. Additional mutations—c.6089 and c.6320—were also included in the minigene, as well as a TAG positive control. A western blot of ADAR1, ADAR2, and GAPDH in the cells is shown in FIG. 17. In FIG. 17, lane 1 is from WT HEK293, lane 2 is from an engineered HEK293 cell line that expresses only ADAR2, and lane 3 is from the ABCA4 mini-gene cell line. The ABCA4 mini-gene was transfected via a piggybac vector into a WT HEK293 cell line (ADAR1 expression) and the mini-gene also expresses ADAR2. Thus, as evidenced in Lane 3, the cells expressed ADAR1 and ADAR2.

Engineered guide RNAs of different lengths and different A/C mismatch positioning—150.125, 150.75. and 100.80—were recombinantly produced. Each of these guide RNAs was further engineered to form structural features upon hybridization to the target ABCA4 RNA. The resulting double stranded substrates formed upon hybridization of the engineered guide RNAs and the target ABCA4 RNA produced an engineered guide-target RNA scaffold that mimicked a drosophila substrate—shown in FIGS. 3 and 4—to varying degrees. gRNAs tested were all linear gRNAs.

A summary of the gRNAs tested is provided in TABLE 9 below. The column in TABLE 9 titled “Structural Features” describes structural features in the double stranded RNA substrate formed upon hybridization of the gRNA to the target RNA. In TABLE 9, % RNA editing of ABCA4 was determined in HEK293 cells expressing ADAR1 and ADAR2 48 hours after engineered guide RNA transfection.

TABLE 9
Engineered gRNAs against ABCA4

| | | | | % |
|---|---|---|---|---|
| Name | | | Structural Features | RNA |
| FIG. | SEQ ID NO | Guide RNA Sequence | (target/guide) | Editing |
| 150.126 | SEQ ID NO: | AATACTCTTGCCTGCTACGGT | 1/0 asymmetric bulge at −2 | 0.67 |
| +1-2 del | 19 | GGCATCCCCTGAGGTCACTG | position (C-) | |
| FIG. 10C | | TGGTGTCCCCAGTGAGCATCT | 1/0 asymmetric bulge +1 | |
| | | TGAATGTGGTTGTATTGCCGG | position (A-) | |
| | | CACCATTCACTCCCAGGAGG | 1/1 A/A mismatch at +52 | |
| | | CCAAAGCACTCTCCAGGGCG | position | |
| | | AACTCACACACAGCCTGTCC | | |
| | | ACTGCTG | | |
| 150.75 | SEQ ID NO: | CAGTGAGCATCTTGAATGTG | 1/0 asymmetric bulge at −2 | 0.67 |
| +1-2 del | 27 | GTTGTATTGCCGGCACCATTC | position (C-) | |
| FIG. 11C | | ACTCCCAGGAGGCCAAAGCA | 1/0 asymmetric bulge +1 | |
| | | CTCTCCAGGGCGAACTCACA | position (A-) | |
| | | CACAGCCTGTCCACTGCTGG | 1/1 A/A mismatch at +52 | |
| | | GCTGGAGGTGCCTGGATAAA | position | |
| | | TCTTGGTTAGTTCATGTAGCC | | |
| | | TTAAGATG | | |
| 101.80 | SEQ ID NO: | CCCCAGTGAGCATCTTGAAT | 1/1 U/G wobble base pair at | 0.5 |
| +1-2 del | 333 | GTGGTTGTATTGCCGGCACC | −21 position | |
| FIG. 9C | | ATTCACTCCCAGGAGGCCAA | 1/0 asymmetric bulge at | |
| | | AGCACTCTCCAGGGCGAACT | position −2 (C-) | |
| | | CACACACAGCCTGTCCACTG | 1/0 asymmetric bulge +1 | |
| | | | position (A-) | |
| | | | 1/1 A/A mismatch at +52 | |
| | | | position | |
| 150.126 | SEQ ID NO: | ATACTCTTGCCTGCTACGGTG | 1/0 asymmetric bulge +1 | 1.33 |
| +1 del | 20 | GCATCCCCTGAGGTCACTGT | position (A-) | |
| FIG. 10D | | GGTGTCCCCAGTGAGCATCTT | 1/1 A/A mismatch at +52 | |
| | | GAATGTGGTTGTATTGCCGG | position | |
| | | CACCATTCACTCCCAGGAGG | | |
| | | CCAAAGCACTCTCCAGGGCG | | |
| | | AACTCGACACACAGCCTGTC | | |
| | | CACTGCTG | | |
| 150.75 | SEQ ID NO: | AGTGAGCATCTTGAATGTGG | 1/0 asymmetric bulge +1 | 0.67 |
| +1 del | 28 | TTGTATTGCCGGCACCATTCA | position (A-) | |
| FIG. 11D | | CTCCCAGGAGGCCAAAGCAC | 1/1 A/A mismatch at +52 | |
| | | TCTCCAGGGCGAACTCGACA | position | |
| | | CACAGCCTGTCCACTGCTGG | | |
| | | GCTGGAGGTGCCTGGATAAA | | |
| | | TCTTGGTTAGTTCATGTAGCC | | |
| | | TTAAGATG | | |
| 100.80 | SEQ ID NO: | CCCAGTGAGCATCTTGAATG | 1_1/0 asymmetric bulge +1 | 1 |
| +1 del | 334 | TGGTTGTATTGCCGGCACCAT | position (A-) | |
| FIG. 9D | | TCACTCCCAGGAGGCCAAAG | 1/1 A/A mismatch at +52 | |
| | | CACTCTCCAGGGCGAACTCG | position | |
| | | ACACACAGCCTGTCCACTG | | |
| 150.126 | SEQ ID NO: | AATACTCTTGCCTGCTACGGT | 1/1 U/G wobble base pair at | 17.33 |
| Full | 21 | GGCATCCCCTGAGGTCACTG | −18 position | |
| mimicry_+ | | TGGTGTCCCCAGTGAGCATCT | 2/1 asymmetric bulge at −14 | |
| 5 del | | TGAATGTGGTTGTATTGCCGG | position (AC-C) | |
| FIG. 10E | | CACCATTCACTCCCAGGAGG | 1/0 asymmetric bulge at −5 | |
| | | CCAAAGCACTCTCCAGTGAG | position (U-) | |
| | | AACTCGGACCACAGCCTCCC | 2/2 symmetric bulge at −1 | |
| | | GCTGCTG | position spanning 0 position | |
| | | | (GA-CG) | |
| | | | 1/1 G/A mismatch at +6 | |
| | | | position | |
| | | | 1/1 C/U mismatch at +8 | |
| | | | position | |
| | | | 1/1 A/A mismatch at +52 | |
| | | | position | |
| 150.75 | SEQ ID NO: | CAGTGAGCATCTTGAATGTG | 1/1 U/G wobble base pair at | 4 |
| Full | 29 | GTTGTATTGCCGGCACCATTC | −18 position | |
| mimicry_ + | | ACTCCCAGGAGGCCAAAGCA | 2/1 asymmetric bulge at −14 | |
| 5 del | | CTCTCCAGTGAGAACTCGGA | position (AC-C) | |
| FIG. 11E | | CCACAGCCTCCCGCTGCTGG | 1/0 asymmetric bulge at −5 | |
| | | GCTGGAGGTGCCTGGATAAA | position (U-) | |
| | | TCTTGGTTAGTTCATGTAGCC | 2/2 symmetric bulge at −1 | |
| | | TTAAGATG | position spanning 0 position | |
| | | | (GA-CG) | |
| | | | 1/1 G/A mismatch at +6 | |
| | | | position | |
| | | | 1/1 C/U mismatch at +8 | |
| | | | position | |
| | | | 1/1 A/A mismatch at +52 | |
| | | | position | |
| 101.80 | SEQ ID NO: | CCCCAGUGAGCAUCUUGAAU | 1/1 U/G wobble base pair at | 18 |
| Full | 11 | GUGGUUGUAUUGCCGGCACC | −21 position | |
| mimicry_ + | | AUUCACUCCCAGGAGGCCAA | 1/1 U/G wobble base pair at | |
| 5 del | | AGCACUCUCCAGUGAGAACU | −18 position | |
| FIG. 9A | | CGGACCACAGCCUCCCGCUG | 2/1 asymmetric bulge at −14 | |
| | | | position (AC-C) | |
| | | | 1/0 asymmetric bulge at −5 | |
| | | | position (U-) | |
| | | | 2/2 symmetric bulge at −1 | |
| | | | position spanning 0 position | |
| | | | (GA-CG) | |
| | | | 1/1 G/A mismatch at +6 | |
| | | | position | |
| | | | 1/1 C/U mismatch at +8 | |
| | | | position | |
| | | | 1/1 A/A mismatch at +52 | |
| | | | position | |
| 150.126 | SEQ ID NO: | AATACTCTTGCCTGCTACGGT | 2/1 asymmetric bulge at −14 | 17.67 |
| Full | 22 | GGCATCCCCTGAGGTCACTG | position (AC-C) | |
| mimicry_+ | | TGGTGTCCCCAGTGAGCATCT | 1/0 asymmetric bulge at −7 | |
| 7 del | | TGAATGTGGTTGTATTGCCGG | position (U-) | |
| FIG. 10F | | CACCATTCACTCCCAGGAGG | 2/2 symmetric bulge at −1 | |
| | | CCAAAGCACTCTCCAGTGAG | position spanning 0 position | |
| | | AACTCGGACACCAGCCTCCC | (GA-CG) | |
| | | ACTGCTG | 1/1 G/A mismatch at +6 | |
| | | | position | |
| | | | 1/1 C/U mismatch at +8 | |
| | | | position | |
| | | | 1/1 A/A mismatch at +52 | |
| | | | position | |
| 150.75 | SEQ ID NO: | CAGTGAGCATCTTGAATGTG | 2/1 asymmetric bulge at −14 | 1 |
| Full | 30 | GTTGTATTGCCGGCACCATTC | position (AC-C) | |
| mimicry_+ 7 | | ACTCCCAGGAGGCCAAAGCA | 1/0 asymmetric bulge at −7 | |
| del | | CTCTCCAGTGAGAACTCGGA | position (U-) | |
| FIG. 11F | | CACCAGCCTCCCACTGCTGG | 2/2 symmetric bulge at −1 | |
| | | GCTGGAGGTGCCTGGATAAA | position spanning 0 position | |
| | | TCTTGGTTAGTTCATGTAGCC | (GA-CG) | |
| | | TTAAGATG | 1/1 G/A mismatch at +6 | |
| | | | position | |
| | | | 1/1 C/U mismatch at +8 | |
| | | | position | |
| | | | 1/1 A/A mismatch at +52 | |
| | | | position | |
| 101.80 | SEQ ID NO: | CCCCAGUGAGCAUCUUGAAU | 1/1 U/G wobble base pair at | 15 |
| Full | 12 | GUGGUUGUAUUGCCGGCACC | −21 position | |
| mimicry_+ 7 | | AUUCACUCCCAGGAGGCCAA | 1/1 U/G wobble base pair at | |
| del | | AGCACUCUCCAGUGAGAACU | −18 position | |
| FIG. 9B | | CGGACACCAGCCUCCCGCUG | 2/1 asymmetric bulge at −14 | |
| | | | position (AC-C) | |
| | | | 1/0 asymmetric bulge at −7 | |
| | | | position (U-) | |
| | | | 2/2 symmetric bulge at −1 | |
| | | | position spanning 0 position | |
| | | | (GA-CG) | |
| | | | 1/1 G/A mismatch at +6 | |
| | | | position | |
| | | | 1/1 C/U mismatch at +8 | |
| | | | position | |
| | | | 1/1 A/A mismatch at +52 | |
| | | | position | |
| 150.126 | SEQ ID NO: | AATACTCTTGCCTGCTACGGT | 2/1 asymmetric bulge at −14 | 16.33 |
| Half | 23 | GGCATCCCCTGAGGTCACTG | position (AC-C) | |
| mimicry_ | | TGGTGTCCCCAGTGAGCATCT | 1/0 symmetric bulge at −5 | |
| bulges_+ 5 | | TGAATGTGGTTGTATTGCCGG | position (U-) | |
| del | | CACCATTCACTCCCAGGAGG | 2/2 symmetric bulge at −1 | |
| FIG. 10G | | CCAAAGCACTCTCCAGGGAG | position spanning 0 position | |
| | | AACTCGGACCACAGCCTCCC | (GA-CG) | |
| | | ACTGCTG | 1/1 G/A mismatch at +6 | |
| | | | position | |
| | | | 1/1 A/A mismatch at +52 | |
| | | | position | |
| 150.75 | SEQ ID NO: | CAGTGAGCATCTTGAATGTG | 2/1 asymmetric bulge at −14 | 2 |
| Half | 31 | GTTGTATTGCCGGCACCATTC | position (AC-C) | |
| mimicry_ | | ACTCCCAGGAGGCCAAAGCA | 1/0 symmetric bulge at −5 | |
| bulges_+ 5 | | CTCTCCAGGGAGAACTCGGA | position (U-) | |
| del | | CCACAGCCTCCCACTGCTGG | 2/2 symmetric bulge at −1 | |
| FIG. 11G | | GCTGGAGGTGCCTGGATAAA | position spanning 0 position | |
| | | TCTTGGTTAGTTCATGTAGCC | (GA-CG) | |
| | | TTAAGATG | 1/1 G/A mismatch at +6 | |
| | | | position | |
| | | | 1/1 A/A mismatch at +52 | |
| | | | position | |
| 101.80 | SEQ ID NO: | CCCCAGUGAGCAUCUUGAAU | 1/1 U/G wobble base pair at | 18.5 |
| Half | 13 | GUGGUUGUAUUGCCGGCACC | −21 position | |
| mimicry_ | | AUUCACUCCCAGGAGGCCAA | 2/1 asymmetric bulge at −14 | |
| bulges_+ 5 | | AGCACUCUCCAGGGAGAACU | position (AC-C) | |
| del | | CGGACCACAGCCUCCCACUG | 1/0 symmetric bulge at −5 | |
| | | | position (U-) | |
| | | | 2/2 symmetric bulge at −1 | |
| | | | position spanning 0 position | |
| | | | (GA-CG) | |
| | | | 1/1 G/A mismatch at +6 | |
| | | | position | |
| | | | 1/1 A/A mismatch at +52 | |
| | | | position | |
| 150.126 | SEQ ID NO: | AATACTCTTGCCTGCTACGGT | 2/1 asymmetric bulge at −14 | 18.33 |
| Half | 24 | GGCATCCCCTGAGGTCACTG | position (AC-C) | |
| mimicry_ | | TGGTGTCCCCAGTGAGCATCT | 1/0 asymmetric bulge at −7 | |
| bulges_+ 7 | | TGAATGTGGTTGTATTGCCGG | position (U-) | |
| del | | CACCATTCACTCCCAGGAGG | 2/2 symmetric bulge at −1 | |
| FIG. 10H | | CCAAAGCACTCTCCAGGGAG | position spanning 0 position | |
| | | AACTCGGACACCAGCCTCCC | (GA-CG) | |
| | | ACTGCTG | 1/1 G/A mismatch at +6 | |
| | | | position | |
| | | | 1/1 A/A mismatch at +52 | |
| | | | position | |
| 150.75 | SEQ ID NO: | CAGTGAGCATCTTGAATGTG | 2/1 asymmetric bulge at −14 | 2.33 |
| Half | 32 | GTTGTATTGCCGGCACCATTC | position (AC-C) | |
| mimicry_ | | ACTCCCAGGAGGCCAAAGCA | 1/0 asymmetric bulge at −7 | |
| bulges_+ | | CTCTCCAGGGAGAACTCGGA | position (U-) | |
| del | | CACCAGCCTCCCACTGCTGG | 2/2 symmetric bulge at −1 | |
| FIG. 11H | | GCTGGAGGTGCCTGGATAAA | position spanning 0 position | |
| | | TCTTGGTTAGTTCATGTAGCC | (GA-CG) | |
| | | TTAAGATG | 1/1 G/A mismatch at +6 | |
| | | | position | |
| | | | 1/1 A/A mismatch at +52 | |
| | | | position | |
| 101.80 | SEQ ID NO: | CCCCAGUGAGCAUCUUGAAU | 1/1 U/G wobble base pair at | 19 |
| Half | 14 | GUGGUUGUAUUGCCGGCACC | −21 position | |
| mimicry_ | | AUUCACUCCCAGGAGGCCAA | 2/1 asymmetric bulge at −14 | |
| bulges_+ 7 | | AGCACUCUCCAGGGAGAACU | position (AC-C) | |
| del | | CGGACACCAGCCUCCCACUG | 1/0 asymmetric bulge at −7 | |
| | | | position (U-) | |
| | | | 2/2 symmetric bulge at −1 | |
| | | | position spanning 0 position | |
| | | | (GA-CG) | |
| | | | 1/1 G/A mismatch at +6 | |
| | | | position | |
| | | | 1/1 A/A mismatch at +52 | |
| | | | position | |
| 150.126 | SEQ ID NO: | ATACTCTTGCCTGCTACGGTG | 1/1 U/G wobble base pair at | 17.33 |
| Half | 335 | GCATCCCCTGAGGTCACTGT | −18 position | |
| mimicry_ | | GGTGTCCCCAGTGAGCATCTT | 1/0 symmetric bulge at −5 | |
| wobbles_+ 5 | | GAATGTGGTTGTATTGCCGG | position (U-) | |
| del | | CACCATTCACTCCCAGGAGG | 2/2 symmetric bulge at −1 | |
| | | CCAAAGCACTCTCCAGTGCG | position spanning 0 position | |
| | | AACTCGGACCACAGCCTGTC | (GA-CG) | |
| | | CGCTGCTG | 1/1 C/U mismatch at +8 | |
| | | | position 1/1 | |
| | | | 1/1 A/A mismatch at +52 | |
| | | | position | |
| 150.75 | SEQ ID NO: | AGTGAGCATCTTGAATGTGG | 1/1 U/G wobble base pair at | 2.67 |
| Half | 33 | TTGTATTGCCGGCACCATTCA | −18 position | |
| mimicry_ | | CTCCCAGGAGGCCAAAGCAC | 1/0 symmetric bulge at −5 | |
| wobbles_+ 5 | | TCTCCAGTGCGAACTCGGAC | position (U-) | |
| del | | CACAGCCTGTCCGCTGCTGG | 2/2 symmetric bulge at −1 | |
| FIG. 11I | | GCTGGAGGTGCCTGGATAAA | position spanning 0 position | |
| | | TCTTGGTTAGTTCATGTAGCC | (GA-CG) | |
| | | TTAAGATG | 1/1 C/U mismatch at +8 | |
| | | | position 1/1 | |
| | | | 1/1 A/A mismatch at +52 | |
| | | | position | |
| 100.80 | SEQ ID NO: | CCCAGUGAGCAUCUUGAAUG | 1/1 U/G wobble base pair at | 14 |
| Half | 15 | UGGUUGUAUUGCCGGCACCA | −18 position | |
| mimicry_ | | UUCACUCCCAGGAGGCCAAA | 1/0 symmetric bulge at −5 | |
| wobbles_+ 5 | | GCACUCUCCAGUGCGAACUC | position (U-) | |
| del | | GGACCACAGCCUGUCCGCUG | 2/2 symmetric bulge at −1 | |
| | | | position spanning 0 position | |
| | | | (GA-CG) | |
| | | | 1/1 C/U mismatch at +8 | |
| | | | position 1/1 | |
| | | | 1/1 A/A mismatch at +52 | |
| | | | position | |
| 150.126 | SEQ ID NO: | ATACTCTTGCCTGCTACGGTG | 1/1 U/G wobble base pair at | 17.66 |
| Half | 336 | GCATCCCCTGAGGTCACTGT | −18 position | |
| mimicry_ | | GGTGTCCCCAGTGAGCATCTT | 1/0 asymmetric bulge at −7 | |
| wobbles_+ 7 | | GAATGTGGTTGTATTGCCGG | position (U-) | |
| del | | CACCATTCACTCCCAGGAGG | 2/2 symmetric bulge at −1 | |
| | | CCAAAGCACTCTCCAGTGCG | position spanning 0 position | |
| | | AACTCGGACACCAGCCTGTC | (GA-CG) | |
| | | CGCTGCTG | 1/1 C/U mismatch at +8 | |
| | | | position 1/1 | |
| | | | 1/1 A/A mismatch at +52 | |
| | | | position | |
| 150.75 | SEQ ID NO: | AGTGAGCATCTTGAATGTGG | 1/1 U/G wobble base pair at | 1 |
| Half | 34 | TTGTATTGCCGGCACCATTCA | -18 position | |
| mimicry_ | | CTCCCAGGAGGCCAAAGCAC | 1/0 asymmetric bulge at -7 | |
| wobbles_+ 7 | | TCTCCAGTGCGAACTCGGAC | position (U-) | |
| del | | ACCAGCCTGTCCGCTGCTGG | 2/2 symmetric bulge at −1 | |
| FIG. 11J | | GCTGGAGGTGCCTGGATAAA | position spanning 0 position | |
| | | TCTTGGTTAGTTCATGTAGCC | (GA-CG) | |
| | | TTAAGATG | 1/1 C/U mismatch at +8 | |
| | | | position 1/1 | |
| | | | 1/1 A/A mismatch at +52 | |
| | | | position | |
| 100.80 | SEQ ID NO: | CCCAGUGAGCAUCUUGAAUG | 1/1 U/G wobble base pair at | 12.5 |
| Half | 16 | UGGUUGUAUUGCCGGCACCA | −18 position | |
| mimicry_ | | UUCACUCCCAGGAGGCCAAA | 1/0 asymmetric bulge at −7 | |
| wobbles_+ 7 | | GCACUCUCCAGUGCGAACUC | position (U-) | |
| del | | GGACACCAGCCUGUCCGCUG | 2/2 symmetric bulge at −1 | |
| | | | position spanning 0 position | |
| | | | (GA-CG) | |
| | | | 1/1 C/U mismatch at +8 | |
| | | | position 1/1 | |
| | | | 1/1 A/A mismatch at +52 | |
| | | | position | |
| 150.126 | SEQ ID NO: | TACTCTTGCCTGCTACGGTGG | 2/2 symmetric bulge at −1 | 15.67 |
| G and C | 58 | CATCCCCTGAGGTCACTGTG | position spanning 0 position | |
| mismatch | | GTGTCCCCAGTGAGCATCTTG | (GA-CG) | |
| FIG. 10B | | AATGTGGTTGTATTGCCGGC | A/A mismatch at +52 | |
| | | ACCATTCACTCCCAGGAGGC | position | |
| | | CAAAGCACTCTCCAGGGCGA | | |
| | | ACTCGGACACACAGCCTGTC | | |
| | | CACTGCTG | | |
| 150.75 | SEQ ID NO: | GTGAGCATCTTGAATGTGGTT | 2/2 symmetric bulge at −1 | 1 |
| G and C | 26 | GTATTGCCGGCACCATTCACT | position spanning 0 position | |
| mismatch | | CCCAGGAGGCCAAAGCACTC | (GA-CG) | |
| FIG. 11B | | TCCAGGGCGAACTCGGACAC | 1/1 A/A mismatch at +52 | |
| | | ACAGCCTGTCCACTGCTGGG | position | |
| | | CTGGAGGTGCCTGGATAAAT | | |
| | | CTTGGTTAGTTCATGTAGCCT | | |
| | | TAAGATG | | |
| 101.80 | SEQ ID NO: | CCCCAGTGAGCATCTTGAAT | 2/2 symmetric bulge at −1 | 9 |
| G and C | 337 (DNA | GTGGTTGTATTGCCGGCACC | position spanning 0 position | |
| mismatch | equivalent | ATTCACTCCCAGGAGGCCAA | (GA-CG) | |
| FIG. 9F | of SEQ ID | AGCACTCTCCAGGGCGAACT | 1/1 A/A mismatch at +52 | |
| | NO: 17) | CGGACACACAGCCTGTCCAC | position | |
| 150.126 | SEQ ID NO: | TACTCTTGCCTGCTACGGTGG | 1/1 A/C mismatch at 0 | 1 |
| Only C | 18 | CATCCCCTGAGGTCACTGTG | position | |
| mismatch | | GTGTCCCCAGTGAGCATCTTG | 1/1 A/A mismatch at +52 | |
| FIG. 10A | | AATGTGGTTGTATTGCCGGC | position | |
| | | ACCATTCACTCCCAGGAGGC | | |
| | | CAAAGCACTCTCCAGGGCGA | | |
| | | ACTCCGACACACAGCCTGTC | | |
| | | CACTGCTG | | |
| 150.75 | SEQ ID NO: | GTGAGCATCTTGAATGTGGTT | 1/1 A/C mismatch at 0 | 1 |
| Only C | 25 | GTATTGCCGGCACCATTCACT | position | |
| mismatch | | CCCAGGAGGCCAAAGCACTC | 1/1 A/A mismatch at +52 | |
| FIG. 11A | | TCCAGGGCGAACTCCGACAC | position | |
| | | ACAGCCTGTCCACTGCTGGG | | |
| | | CTGGAGGTGCCTGGATAAAT | | |
| | | CTTGGTTAGTTCATGTAGCCT | | |
| | | TAAGATG | | |
| 100.80 | SEQ ID NO: | CCCAGTGAGCATCTTGAATG | 1/1 A/C mismatch at 0 | 1 |
| Only C | 338 | TGGTTGTATTGCCGGCACCAT | position | |
| mismatch | | TCACTCCCAGGAGGCCAAAG | 1/1 A/A mismatch at +52 | |
| FIG. 9E | | CACTCTCCAGGGCGAACTCC | position | |
| | | GACACACAGCCTGTCCACT | | |

HEK293 cells were transfected with plasmids containing the engineered guide RNAs. The 150.125 and 150.75 engineered guide RNAs were transfected in biological triplicate. The 100.80 engineered guide RNAs were transfected in biological duplicate. The target RNA was isolated and collected within 48 hours of transfection, converted into cDNA, and then sequenced via Sanger Sequencing.

FIG. 21 includes an example of the Sanger sequencing reads of the target RNA after transfections with the 150.125 guides, including SEQ ID NO: 21 (left) and SEQ ID NO: 18 (right). NGS data may be further collected to inform off-target editing.

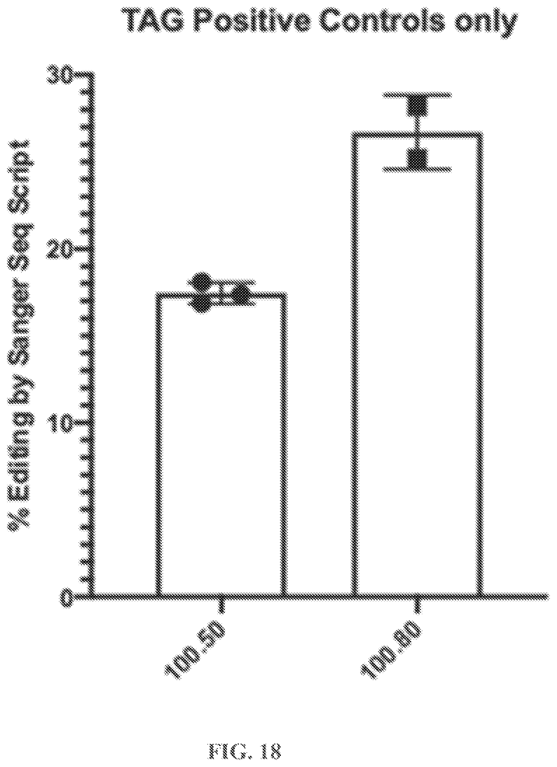

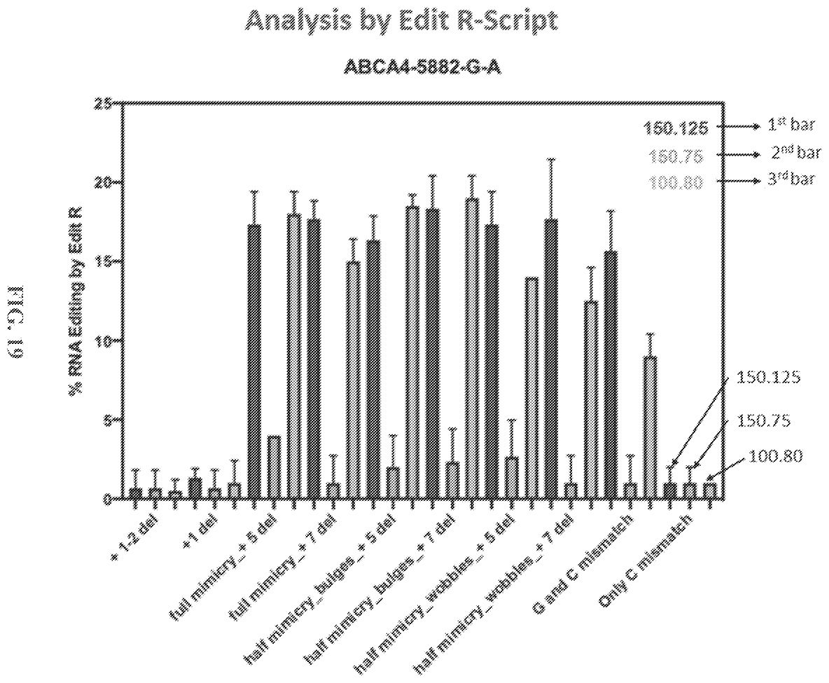

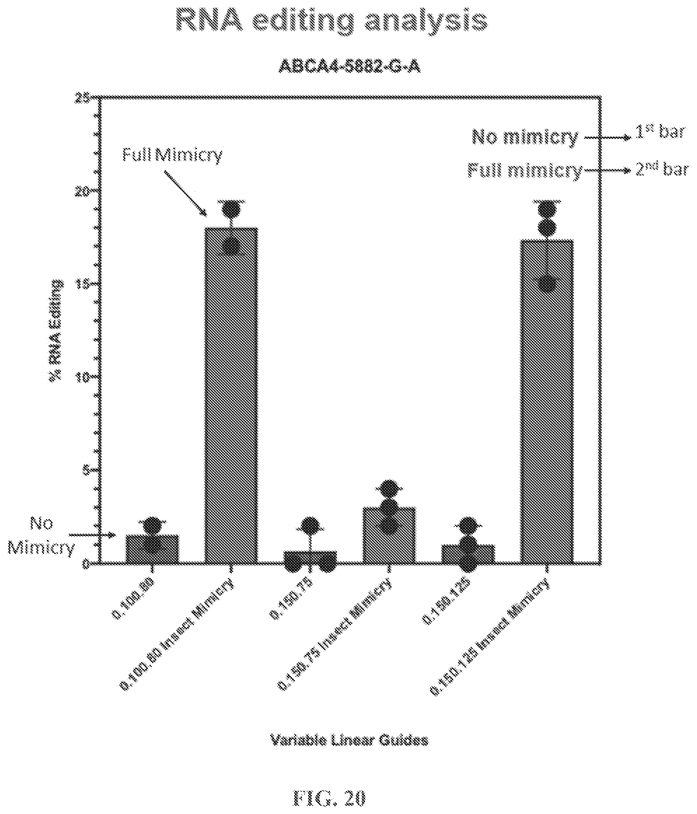

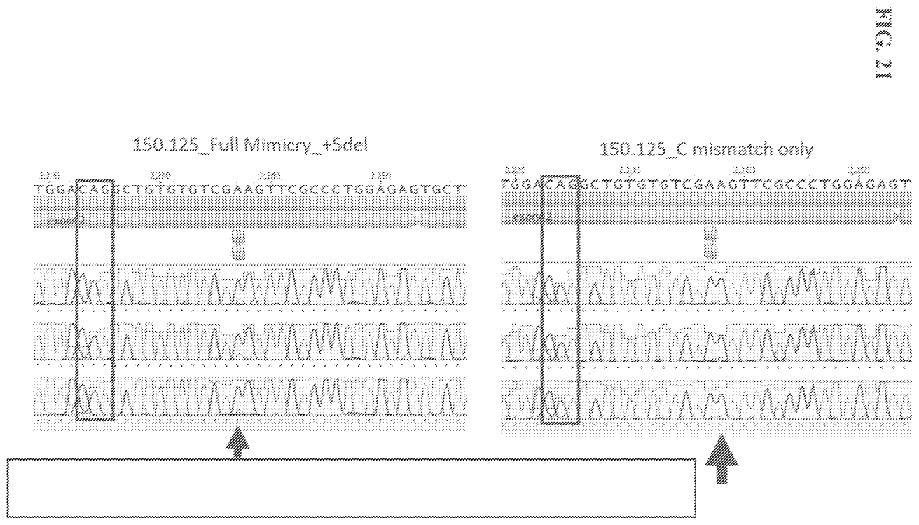

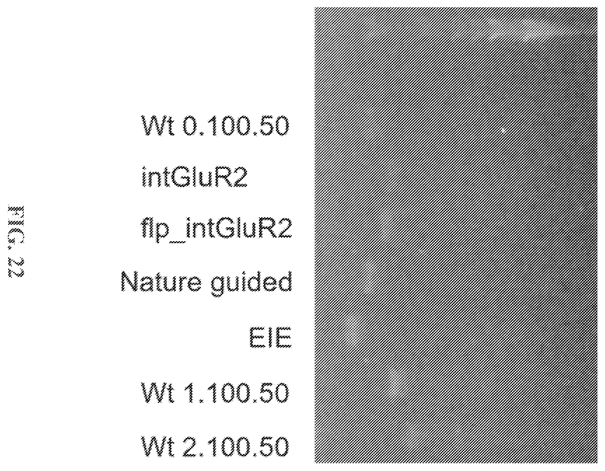

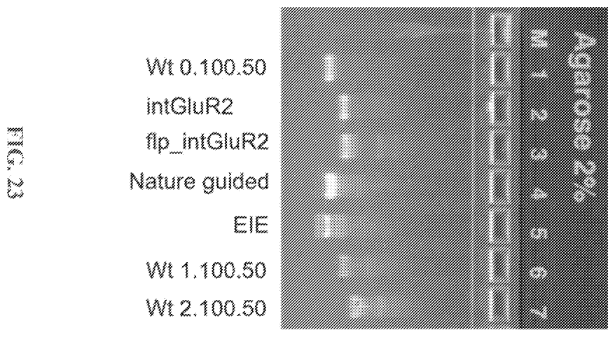

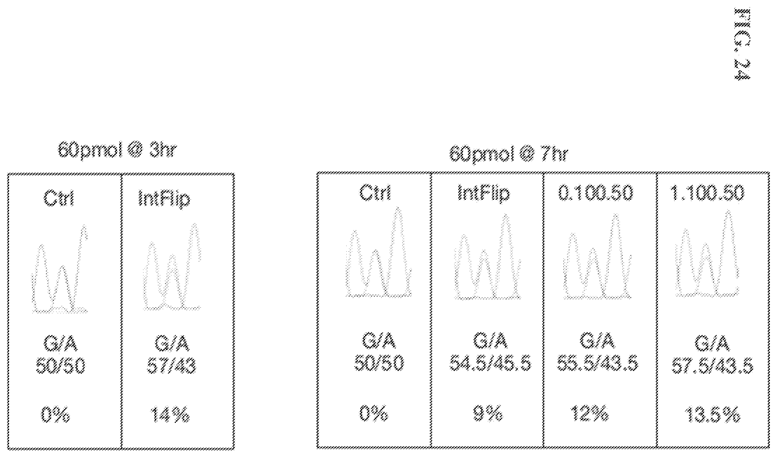

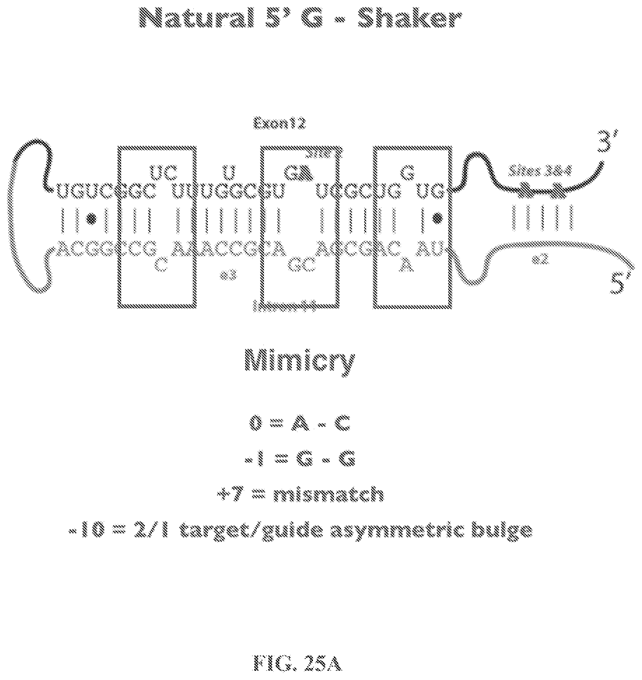

FIG. 18 shows the percent editing of the adenosine in TAG in ABCA4 as a positive control, as determined by Sanger Sequencing. As demonstrated in FIG. 18, guide RNAs are capable of editing adenosines in ABCA4, thus, a challenge in editing the c.5882 mutation in particular is the local sequence context—namely, the 5′G immediately upstream of the target adenosine. Each engineered guide RNA is shown on the x-axis of FIG. 19. Depictions of the structural features of several of the engineered guides are provided in FIG. 9-FIG. 11. Said structural features are also be described in FIG. 6A, FIG. 7, and FIG. 8. FIG. 19 shows the percent editing of the c.5882 mutation in the ABCA4 minigene achieved by the engineered guide RNAs that were tested. FIG. 20 shows a comparison of the % RNA editing achieved by three engineered guide RNAs, comparing versions of the engineered guide RNAs where, upon hybridization to target ABCA4 RNA, the double stranded RNA substrate has no structural features beyond an A/C mismatch at the target A to be edited to versions of the engineered guide RNAs where, upon hybridization to target ABCA4 RNA, the double stranded RNA substrate has various structural features in addition to the A/C mismatch (SEQ ID NO: 11, SEQ ID NO: 29, and SEQ ID NO: 21). As seen in FIG. 20, guide RNAs engineered to form structural features upon hybridization to ABCA4 RNA (e.g., asymmetrical bulges, a G/G mismatch at the 5′G of the target adenosine to be edited, wobble base pairs, etc.) facilitated higher levels of ADAR-mediated RNA editing when compared to engineered guide RNAs with no structural features beyond the A/C mismatch at the target adenosine to be edited.

### Promoter, RNA Elements, and Dose Dependency

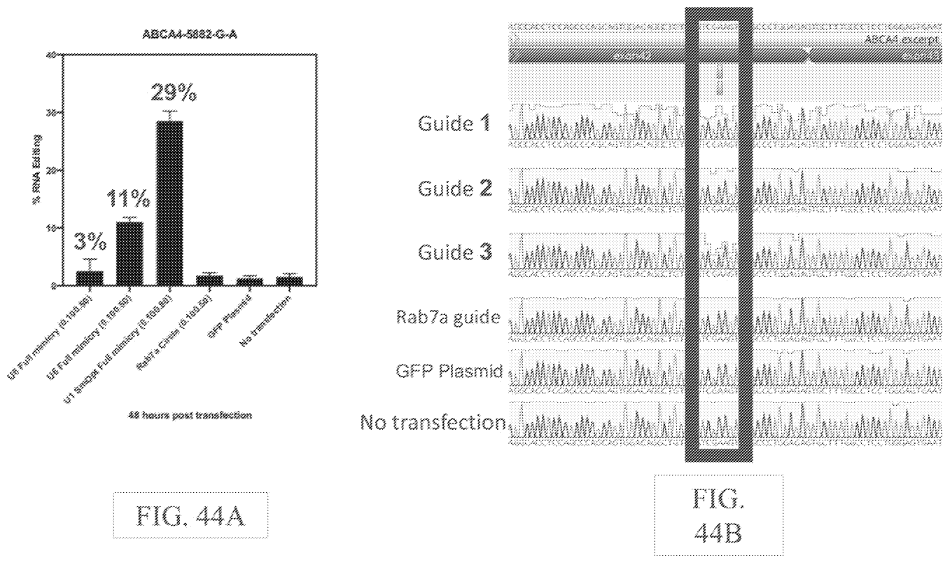

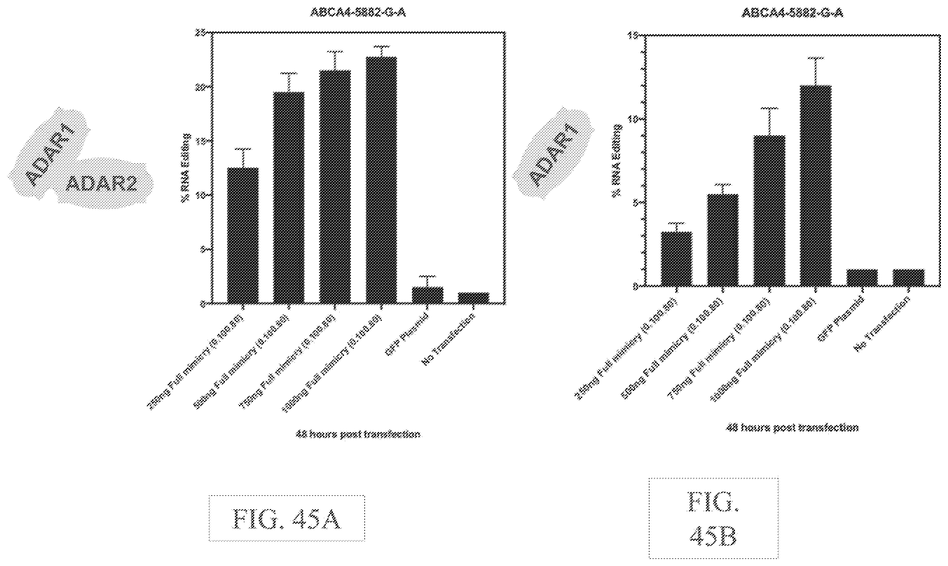

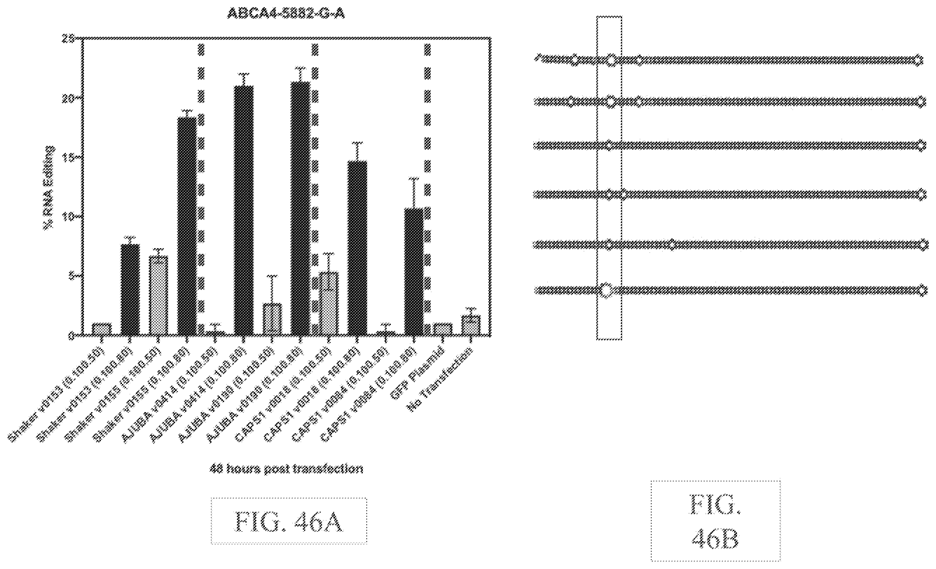

An initial set of experiments demonstrated the improvement in RNA editing of ABCA4 observed in engineered guide RNAs incorporating an SmOPT sequence (AATTTTTGGAG; SEQ ID NO: 390) and a U7 hairpin sequence (CAGGTTTTCTGACTTCGGTCGGAAAACCCCT; SEQ ID NO: 389), depicted below as SEQ ID NO: 339-341 of TABLE 10. HEK293 cells naturally expressing ADAR1 were transfected with a piggyBac vector carrying an ABCA4 minigene having the 5882 G->A mutation and ADAR2. Engineered guide RNAs tested included two U6 driven engineered guide RNAs (“U6 Full mimicry 0.100250” and “U6 Full mimicry 0.10080”) and a driven engineered guide RNA containing the SmOPT sequence and U7 hairpin sequence (“UT Full mimicry 0.100.80”). Negative controls included a circular guide RNA to a different gene (Rab7A), GFP plasmid alone, and no transfection. As shown in FIG. 44A and FIG. 44B, the inclusion of the SmOPT sequence and a U7 hairpin increased RNA editing.

A summary of each engineered guide RNA is described below in TABLE 10. The column in TABLE 10 titled “Structural Features” describes structural features in the double stranded RNA substrate formed upon hybridization of the gRNA to the target RNA.

TABLE 10
Anti-ABCA4 Engineered Guide RNAs

| | | Engineered Guide RNA | | % RNA |
|---|---|---|---|---|
| SEQ ID NO | ID | Sequence | Structural Features | Editing |
| SEQ ID NO: 339 | U6 Full mimicry | GCCGGCACCATTCACTCCCAG | 1/1 U/G wobble base pair | 3 |
| | 0.100.50 | GAGGCCAAAGCACTCTCCAG | at −18 position | |
| | | TGAGAACTCGGACCACAGCC | 2/1 asymmetric bulge at | |
| | | TCCCGCTGCTGGGCTGGAGGT | −14 position (AC-C) | |
| | | GCCTGGATAAATCTTGGT | 1/0 asymmetric bulge at | |
| | | | −5 position (U-) | |
| | | | 2/2 symmetric bulge at | |
| | | | −1 position spanning 0 | |
| | | | position (GA-CG) | |
| | | | 1/1 G/A mismatch at | |
| | | | +6 position | |
| | | | 1/1 C/U mismatch at | |
| | | | +8 position | |
| SEQ ID NO: 340 | U6 Full mimicry | CCCCAGUGAGCAUCUUGAAU | 1/1 U/G wobble base pair | 11 |
| | 0.101.80 | GUGGUUGUAUUGCCGGCACC | at −18 position | |
| | | AUUCACUCCCAGGAGGCCAA | 2/1 asymmetric bulge at | |
| | | AGCACU | −14 position (AC-C) | |
| | | | 1/0 asymmetric bulge at | |
| | | | −5 position (U-) | |
| | | | 2/2 symmetric bulge at | |
| | | | −1 position spanning 0 | |
| | | | position (GA-CG) | |
| | | | 1/1 G/A mismatch at | |
| | | | +6 position | |
| | | | 1/1 C/U mismatch at | |
| | | | +8 position | |
| | | | 1/1 A/A mismatch at | |
| | | | +52 position | |
| SEQ ID NO: | U1/SmOPT_U7 | CCCCAGUGAGCAUCUUGAAU | 1/1 U/G wobble base pair | 29 |
| 341 | hairpin Full | GUGGUUGUAUUGCCGGCACC | at −18 position | |
| | mimicry 0.101.80 | AUUCACUCCCAGGAGGCCAA | 2/1 asymmetric bulge at | |
| | | AGCACUgtggAATTTTTGGAG | −14 position (AC-C) | |
| | | CAGGTTTTCTGACTTCGGTCG | 1/0 asymmetric bulge at | |
| | | GAAAACCCCT | −5 position (U-) | |
| | | | 2/2 symmetric bulge at | |
| | | | −1 position spanning 0 | |
| | | | position (GA-CG) | |
| | | | 1/1 G/A mismatch at | |
| | | | +6 position | |
| | | | 1/1 C/U mismatch at | |
| | | | +8 position | |
| | | | 1/1 A/A mismatch at | |
| | | | +52 position | |

Subsequent experiments evaluated the dose dependence of engineered guide RNAs targeting the ABCA4 5882 G->A mutation, where polynucleotides encoding the engineered guide RNA also encoded for a SmOPT sequence and a U7 hairpin sequence. FIG. 45A shows percent RNA editing in cells by ADAR1 and ADAR2 for multiple doses of constructs encoding an engineered guide RNA targeting a mutation in ABCA4, a SmOPT sequence, and a U7 hairpin sequence, where expression is driven by a U1 promoter. As described above, HEK293 cells naturally express ADAR1. For FIG. 45A, HEK293 cells were transfected with a piggyBac vector carrying an ABCA4 minigene having the 5882 G->A mutation and ADAR2. FIG. 45B shows percent RNA editing in cells by ADAR1 for multiple doses of constructs encoding a guide RNA targeting a mutation in ABCA4, a SmOPT sequence, and a U7 hairpin, where expression is driven by a U1 promoter. HEK293 cells were transfected with a piggyBac vector carrying just the ABCA4 minigene having the 5882 G->A mutation. In both FIG. 45A and FIG. 45B, plasmids encoding the guide RNA, SmOPT sequence, and U7 hairpin were dosed at 250 ng, 500 ng, 750 ng, or 1000 ng. A GFP plasmid and no transfection served as negative controls. Results showed a dose dependency in percent RNA editing of the ABCA4 5882 G->A mutation.

Further Modification with Internal Loops

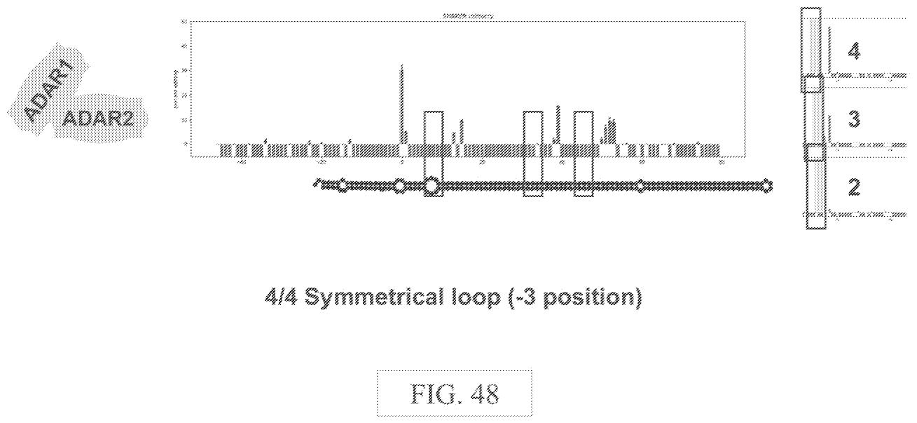

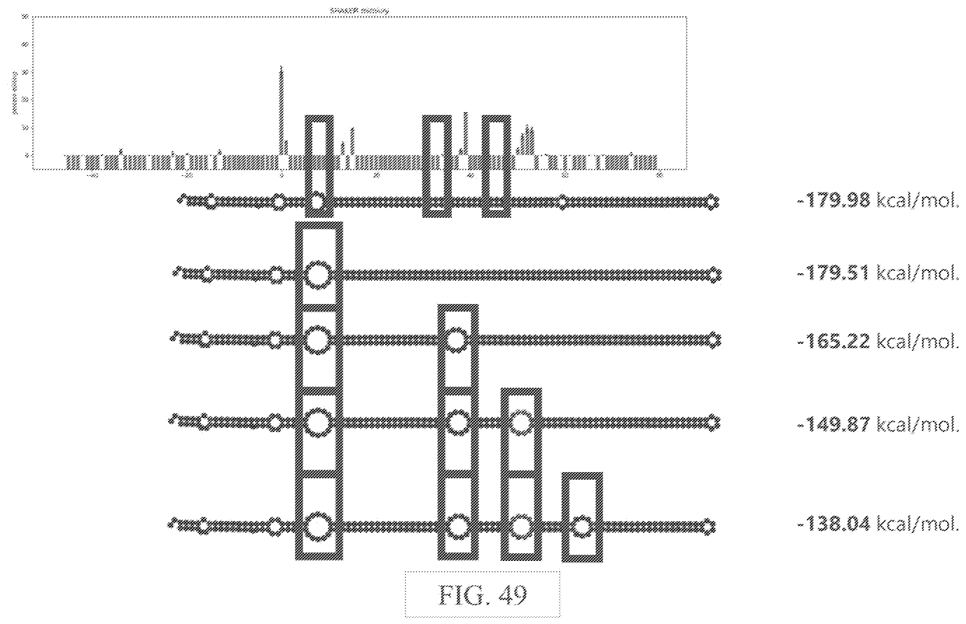

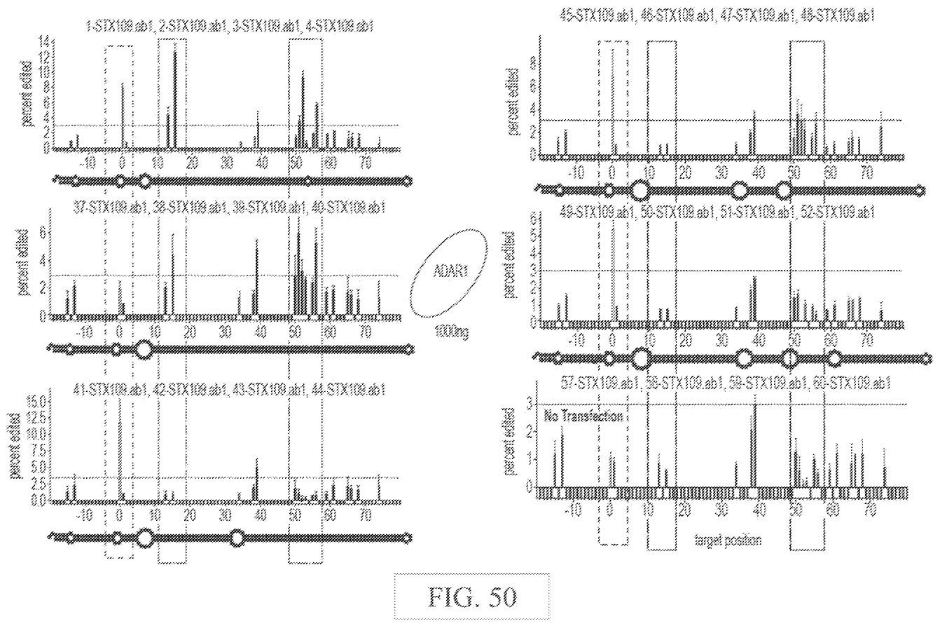

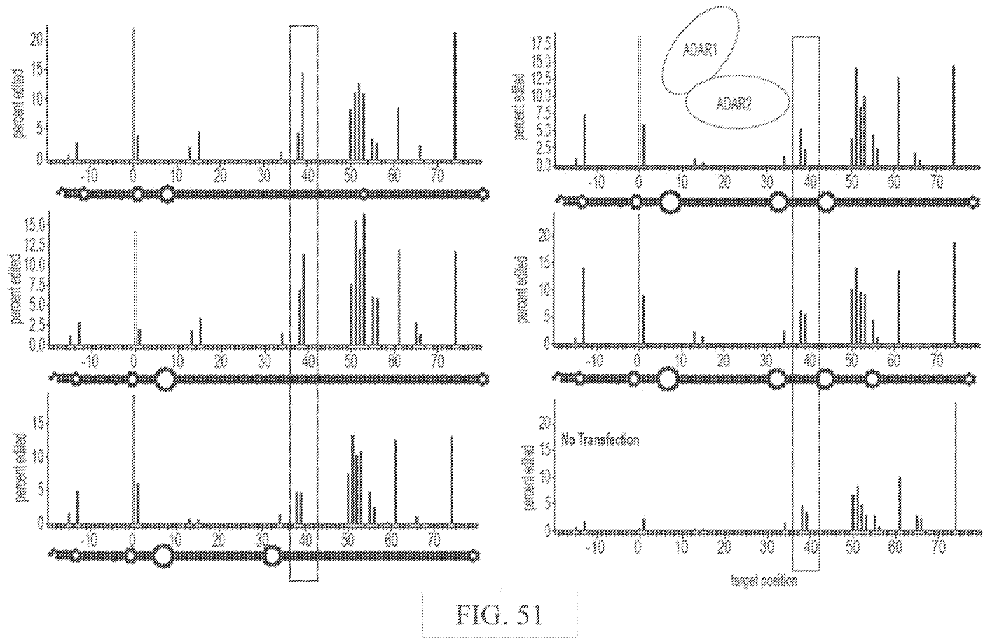

FIG. 48-FIG. 51 shows structures of engineered guide RNAs that were further engineered with additional symmetric 4/4 internal loops placed near areas of off-target editing activity in order to reduce off-target editing. A summary of each engineered guide RNA is described below in TABLE 11. The underlined sequence is the SmOPT sequence and the sequence immediately following the underlined sequence is the U7 hairpin. The italicized sequence is the engineered guide RNA sequence.

TABLE 11
Guide RNA Sequences against ABCA4

| | | | Structural Features | % RNA |
|---|---|---|---|---|
| SEQ ID NO | Name | Sequence | (target/guide) | Editing |
| SEQ ID NO: 292 | ABCA4 U1 | CCCCAGTGAGCATCTT | 1/1 U/G wobble base pair at | ADAR1:8 |
| | SmOPT Full | GAATGTGGTTGTATTG | −18 position | ADAR1/ |
| | mimicry | CCGGCACCATTCACTC | 2/1 asymmetric bulge at −14 | ADAR2:22 |
| | | CCAGGAGGCCAAAGCA | position (AC-C) | |
| | | CTCTCCAGTGAGAACT | 1/0 asymmetric bulge at −5 | |
| | | CGGACCACAGCCTCCC | position (U-) | |
| | | GCTgtgg | 2/2 symmetric bulge at −1 | |
| | | AATTTTTGGAG | position spanning 0 position | |
| | | CAGGTTTTCTGACTTC | (GA-CG) | |
| | | GGTCGGAAAACCCCT | 1/1 G/A mismatch at +6 | |
| | | | position | |
| | | | 1/1 C/U mismatch at +8 | |
| | | | position | |
| | | | 1/1 A/A mismatch at +52 | |
| | | | position | |
| SEQ ID NO: 293 | ABCA4 U1 | CCCCAGTGAGCATCTT | 1/1 U/G wobble base pair at | ADAR1:2 |
| | SmOPT Full | GAATGTGGTTGTTTTG | −21 position | ADAR1/ |
| | mimicry + | CCGGCACCATTCACTC | 1/1 U/G wobble base pair at | ADAR2:14 |
| | 1 4/4 | CCAGGAGGCCAAAGCA | −18 position | |
| | Internal | CTCTCCTCTGAGAACT | 2/1 asymmetric bulge at −14 | |
| | symmetric | CGGACCACAGCCTCCC | position (AC-C) | |
| | loop | GCTGgtgg | 1/0 asymmetric bulge at −5 | |
| | | AATTTTTGGAG | position (U-) | |
| | | CAGGTTTTCTGACTTC | 2/2 symmetric bulge at −1 | |
| | | GGTCGGAAAACCCCT | position spanning 0 position | |
| | | | (GA-CG) | |
| | | | 5/5 symmetric internal loop | |
| | | | at +6 position (GCCCU- | |
| | | | UCUGA) | |
| SEQ ID NO: 294 | ABCA4 U1 | CCCCAGTGAGCATCTT | 1/1 U/G wobble base pair at | ADAR1:11 |
| | SmOPT Full | GAATGTGGTTGTTTTG | −21 position | ADAR1/ |
| | mimicry + | CCGGCACCATTCAGAG | 1/1 U/G wobble base pair at | ADAR2:19 |
| | 2 4/4 | GCAGGAGGCCAAAGC | −18 position | |
| | Internal | ACTCTCCTCTGAGAAC | 2/1 asymmetric bulge at −14 | |
| | symmetric | TCGGACCACAGCCTCC | position (AC-C) | |
| | loop | CGCTGgtgg | 1/0 asymmetric bulge at −5 | |
| | | AATTTTTGGAG | position (U-) | |
| | | CAGGTTTTCTGACTTC | 2/2 symmetric bulge at −1 | |
| | | GGTCGGAAAACCCCT | position spanning 0 position | |
| | | | (GA-CG) | |
| | | | 5/5 symmetric internal loop | |
| | | | at +6 position (GCCCU- | |
| | | | UCUGA) | |
| | | | 4/4 symmetric bulge at +32 | |
| | | | position (GGAG-GAGG) | |
| SEQ ID NO: 295 | ABCA4 U1 | CCCCAGTGAGCATCTT | 1/1 U/G wobble base pair at | ADAR1:7 |
| | SmOPT Full | GAATGTGGTTGTTTTG | −21 position | ADAR1/ |
| | mimicry + | CGCCGACCATTCAGAG | 1/1 U/G wobble base pair at | ADAR2:18 |
| | 3 4/4 | GCAGGAGGCCAAAGC | −18 position | |
| | Internal | ACTCTCCTCTGAGAAC | 2/1 asymmetric bulge at −14 | |
| | symmetric | TCGGACCACAGCCTCC | position (AC-C) | |
| | loop | CGCTGgtgg | 1/0 asymmetric bulge at −5 | |
| | | AATTTTTGGAG | position (U-) | |
| | | CAGGTTTTCTGACTTC | 2/2 symmetric bulge at −1 | |
| | | GGTCGGAAAACCCCT | position spanning 0 position | |
| | | | (GA-CG) | |
| | | | 5/5 symmetric internal loop | |
| | | | at +6 position (GCCCU- | |
| | | | UCUGA) | |
| | | | 4/4 symmetric bulge at +32 | |
| | | | position (GGAG-GAGG) | |
| | | | 4/4 symmetric bulge at +44 | |
| | | | position (GCCG-GCCG) | |
| SEQ ID NO: 296 | ABCA4 U1 | CCCCAGTGAGCATCTT | 1/1 U/G wobble base pair at | ADAR1:5 |
| | SmOPT Full | GAATGTCCATGTTTTGC | −21 position | ADAR1/ |
| | mimicry + | GCCGACCATTCAGAGG | 1/1 U/G wobble base pair at | ADAR2:21 |
| | 4 4/4 | CAGGAGGCCAAAGCAC | −18 position | |
| | Internal | TCTCCTCTGAGAACTC | 2/1 asymmetric bulge at −14 | |
| | symmetric | GGACCACAGCCTCCCG | position (AC-C) | |
| | loop | CTGgtgg | 1/0 asymmetric bulge at −5 | |
| | | AATTTTTGGAG | position (U-) | |
| | | CAGGTTTTCTGACTTC | 2/2 symmetric bulge at −1 | |
| | | GGTCGGAAAACCCCT | position spanning 0 position | |
| | | | (GA-CG) | |
| | | | 5/5 symmetric internal loop | |
| | | | at +6 position (GCCCU- | |
| | | | UCUGA) | |
| | | | 4/4 symmetric bulge at +32 | |
| | | | position (GGAG-GAGG) | |
| | | | 4/4 symmetric bulge at +44 | |
| | | | position (GCCG-GCCG) | |
| | | | 3/3 symmetric bulge at +56 | |
| | | | position (ACC-CCA) | |

### Example 5

### In Vitro Transcribed (IVT) LRRK2-Targeting Engineered Guide RNA

This example describes in vitro transcribed (IVT) LRRK2-targeting engineered guide RNAs. gBlocks™ Gene Fragments were purchased from IDT and used to generate IVT engineered guide RNAs (TABLE 12). Formatting of sequences in TABLE 12 indicates various elements of each DNA construct including non-transcribed T7 promoter elements (lowercase), primer binding sequence (underlined); GluR2 recruiting domain (italicized). A denotes a nucleotide mismatch; * denotes a nucleotide that will form a bulge; # denotes a nucleotide that will form an internal loop; underlining denotes a nucleotide that will form part of a hairpin. The column in TABLE 12 titled “Structural Features” describes structural features in the double stranded RNA substrate formed upon hybridization of the gRNA to the target RNA.

TABLE 12
Constructs for In Vitro Transcribed LRKK2 Engineered Guide RNAs

| | | | Structural Features | % RNA |
|---|---|---|---|---|
| SEQ ID NO | Guide RNA | Sequence | (target/guide) | Editing |
| SEQ ID NO: | LRRK2_0.100.50 | (atatgctaatacgactcactata | 1/1 A/C mismatch at 0 | 12% |
| 35 | | g)GTGCCCTCTGATG | position | |
| | | TTTTTATCCCCATTC | | |
| | | TACAGCAGTACTGA | | |
| | | GCAATGCCGTAGTC | | |
| | | AGCAATCTTTGCAA | | |
| | | TGATGGCAGCATTG | | |
| | | GGATACAGTGTGAA | | |
| | | AA | | |
| SEQ ID NO: | LRRK2_1.100.50 | (atatgctaatacgactcactata | 1/1 A/C mismatch at 0 | 13.5% |
| 36 | | g)GTGCCCTCTGATG | position | |
| | | TTTTTATCCCCATTC | GluR2 recruiting | |
| | | TACAGCAGTACTGA | domain at +50 position | |
| | | GCAATGCCGTAGTC | | |
| | | AGCAATCTTTGCAA | | |
| | | TGATGGCAGCATTG | | |
| | | GGATACAGTGTGAA | | |
| | | AAGTGGAATAGTATA | | |
| | | ACAATATGCTAAATG | | |
| | | TTGTTATAGTATCCCA | | |
| | | C | | |
| SEQ ID NO: | LRRK2_2.100.50 | atatgctaatacgactcactatag | 1/1 A/C mismatch at 0 | 8% |
| 37 | | GTGGAATAGTATAAC | position | |
| | | AATATGCTAAATGTT | GluR2 recruiting | |
| | | GTTATAGTATCCCAC | domain at +50 position | |
| | | GTGCCCTCTGATGT | GluR2 recruiting | |
| | | TTTTATCCCCATTCT | domain at −50 position | |
| | | ACAGCAGTACTGAG | | |
| | | CAATGCCGTAGTCA | | |
| | | GCAATCTTTGCAAT | | |
| | | GATGGCAGCATTGG | | |
| | | GATACAGTGTGAAA | | |
| | | AGTGGAATAGTATAA | | |
| | | CAATATGCTAAATGTT | | |
| | | GTTATAGTATCCCAC | | |
| SEQ ID NO: | LRRK2_Natguide | atatgctaatacgactcactatag | 3/2 asymmetric bulge at | 5% |
| 393 | | GTGCCCTCTGATGT | position 0 to −3 | |
| | | TTTTATCCCCATTCG | 0/1 asymmetric bulge at | |
| | | GTTACAGTAATGAG | +4 position | |
| | | CAAATGAGCAAATG | 1/1 mismatch at base | |
| | | CCGAGTCAGCAAGA | +12 position | |
| | | TTTGCTGGTTGGGC | 5/5 symmetric loop at | |
| | | AGCATTGGGATACA | +18 position | |
| | | GTGTGAAAA | 2/2 symmetric bulge at | |
| | | | −12 position | |
| | | | 6/6 symmetric loop at | |
| | | | −19 position | |
| SEQ ID NO: | LRRK2_EIE | atatgctaatacgactcactatag | 2/2 bulge at −1 to 0 | 6% |
| 39 | | AAGGACGGGTCGTA | positions | |
| | | CTGAGCAATGCCCT | 0/9 asymmetric loop at | |
| | | AGTCAAAGTGGACA | −6 position | |
| | | GCAATCTTTGCAAC | 1/1 mismatch at −21 | |
| | | GATGGCAGCATCGG | position | |
| | | GATACACCTGTGAC | 1/1 mismatch at −33 | |
| | | TAA | position | |
| | | | 1/2 asymmetric bulge at | |
| | | | −42 position | |
| SEQ ID NO: | LRRK2_ | atatgctaatacgactcactatag | A/C mismatch at 0 | 14% |
| 40 | FlipIntGlu | GTGCCCTCTGATGT | position | |
| | | TCTTATCCCCATTCC | 1/1 mismatch at +21 | |
| | | ACAGCAGTACTGAG | position | |
| | | CAATGCCGTAGTCA | 1/1 mismatch at +34 | |
| | | CACCCTATGATATTG | position | |
| | | TTGTAAATCGTATAAC | GluR2 Recruiting | |
| | | AATATGATAAGGTGG | domain at −7 position | |
| | | CAATCTTCGCAATG | 1/1 mismatch at −16 | |
| | | ATGGCAGCATTGGG | position | |
| | | ACACAGTGTGAAAA | 1/1 mismatch at −38 | |
| | | | position | |
| SEQ ID NO: | LRRK2_IntGluR2 | atatgctaatacgactcactatag | A/C mismatch at 0 | 9% |
| 41 | | GTGCCCTCTGATGT | position | |
| | | TCTTATCCCCATTCC | 1/1 mimatch at +21 | |
| | | ACAGCAGTACTGAG | position | |
| | | GATGGCAGCATTGG | 1/1 mismatch at +34 | |
| | | CAATGCCGTAGTCA | position | |
| | | GTGGAATAGTATAAC | GluR2 Recruiting | |
| | | AATATGCTAAATGTT | domain at −7 position | |
| | | GTTATAGTATCCCAC | 1/1 mismatch at −16 | |
| | | GCAATCTTCGCAAT | position | |
| | | GATACAGTGTGAAA | | |
| | | A | | |
| SEQ ID NO: | LRRK2 EIEv2 | atatgctaatacgactcactatag | A/C mismatch at 0 | 5% |
| 42 | | AAGGACGGGTCGTA | position | |
| | | CTGAGCAATGCCGT | 0/9 asymmetric loop at | |
| | | AGTCAAAGTGGACA | −6 position | |
| | | GCAATCTTTGCAAC | 1/1 mismatch at −21 | |
| | | GATGGCAGCATCGG | position | |
| | | GATACACCTGTGAC | 1/1 mismatch at −33 | |
| | | TAA | position | |
| | | | 1/2 asymmetric bulge at | |
| | | | −42 position | |

The IVT was carried out using the reagents, quantities, and concentrations described in TABLE 13. IVT templates were made for all engineered guide RNAs following Q5 PCR protocol (60C annealing) followed by confirmation via gel electrophoresis (FIG. 22).

TABLE 13
Exemplary IVT Protocol

| Reagent | Quantity | Concentration |
|---|---|---|
| Nuclease-free water | 5 μl | |
| 10X Reaction Buffer | 2 μl | |
| ATP (100 mM) | 2 μl | 10 mM final |
| GTP (100 mM) | 2 μl | 10 mM final |
| UTP (100 mM) | 2 μl | 10 mM final |
| CTP (100 mM) | 2 μl | 10 mM final |
| Template DNA | 3 μl | 1 μg |
| T7 RNA Polymerase Mix | 2 μl | |
| Total reaction volume | 20 μl | |

In brief, the IVT protocol shown in TABLE 13 was utilized to generate IVT guide RNA. Reagents were mixed and incubated at 37° C. overnight (overnight IVT generally gives a great yield). For DNase treatment, 70 μl nuclease-free water, 10 μl of 10× DNase I Buffer, and 2 μl of DNase I (RNase-free) were mixed and incubated for 30 minutes at 37° C. Purified IVT produced polynucleotide RNA was adjusted to 1 μg/ul (˜25 nmol).

TABLE 14
IVT Primers

| SEQ ID NO | Primer | Sequence |
|---|---|---|
| SEQ ID NO: 43 | LRRK2_RP_ | TTTTCACACTGTATCCCAATG |
| | GEN | |
| SEQ ID NO: 44 | LRRK2_RP_ | TTAGTCACAGGTGTATCCC |
| | EIE | |
| SEQ ID NO: 43 | LRRK2_RP_ | TTTTCACACTGTATCCCAATG |
| | INTG2 | |

Engineered guide RNAs are shown in TABLE 15. These engineered guide RNAs can revert a single base pair mutation at position 6190 of the LRRK2 mRNA sequence. The formatting of the sequences in TABLE 15 indicated various elements of each construct: Recruiting sequences (GluR2) are italicized. \hat{ } denotes a nucleotide mismatch; * denotes a nucleotide that will form a bulge; # denotes a nucleotide that will form an internal loop; underlining denotes a nucleotide that will form part of a hairpin. The column in TABLE 15 titled “Structural Features” describes structural features in the double stranded RNA substrate formed upon hybridization of the gRNA to the target RNA.

TABLE 15
Anti-LRRK2 Engineered Guide RNAs and Target mRNA Sequences

| SEQ | | | | |
|---|---|---|---|---|
| ID | | Engineered Guide | Structural Features | Target mRNA |
| NO | ID | RNA Sequence | (target/guide) | Sequence |
| SEQ | LRRK2_0.100.50 | GUGCCCUCUGAU | 1/1 A/C mismatch at | UUUUCACACUGU |
| ID | | GUUUUUAUCCCC | 0 position | AUCCCAAUGCUG |
| NO: | | AUUCUACAGCAG | | CCAUCAUUGCAA |
| 46 | | UACUGAGCAAUG | | AGAUUGCUGACU |
| | | CCGUAGUCAGCA | | ACAGCAUUGCUC |
| | | AUCUUUGCAAUG | | AGUACUGCUGUA |
| | | AUGGCAGCAUUG | | GAAUGGGGAUAA |
| | | GGAUACAGUGUG | | AAACAUCAGAGG |
| | | AAAA | | GCAC (SEQ ID NO: |
| | | | | 53) |
| SEQ | LRRK2_1.100.50 | GUGCCCUCUGAU | 1/1 A/C mismatch at | (SEQ ID NO: 53) |
| ID | | GUUUUUAUCCCC | 0 position | |
| NO: | | AUUCUACAGCAG | GluR2 recruiting | |
| 47 | | UACUGAGCAAUG | domain at +50 | |
| | | CCGUAGUCAGCA | position | |
| | | AUCUUUGCAAUG | | |
| | | AUGGCAGCAUUG | | |
| | | GGAUACAGUGUG | | |
| | | AAAAGUGGAAUA | | |
| | | GUAUAACAAUAUG | | |
| | | CUAAAUGUUGUUA | | |
| | | UAGUAUCCCAC | | |
| SEQ | LRRK2 2.100.50 | GUGGAAUAGUAUA | 1/1 A/C mismatch at | (SEQ ID NO: 53) |
| ID | | ACAAUAUGCUAAA | 0 position | |
| NO: | | UGUUGUUAUAGU | GluR2 recruiting | |
| 48 | | AUCCCACGUGCCC | domain at +50 | |
| | | UCUGAUGUUUUU | position | |
| | | AUCCCCAUUCUA | GluR2 recruiting | |
| | | CAGCAGUACUGA | domain at −50 | |
| | | GCAAUGCCGUAG | position | |
| | | UCAGCAAUCUUU | | |
| | | GCAAUGAUGGCA | | |
| | | GCAUUGGGAUAC | | |
| | | AGUGUGAAAAGU | | |
| | | GGAAUAGUAUAAC | | |
| | | AAUAUGCUAAAUG | | |
| | | UUGUUAUAGUAU | | |
| | | CCCAC | | |
| SEQ | LRRK2_Natguide | GUGCCCUCUGAU | 3/2 asymmetric bulge | (SEQ ID NO: 53) |
| ID | | GUUUUUAUCCCC | at position 0 to −3 | |
| NO: | | AUUCGGUUACAG | 0/1 asymmetric bulge | |
| 49 | | UAAUGAGCAAAU | at +4 position | |
| | | GCCGAGUCAGCA | 1/1 mismatch at | |
| | | AGAUUUGCUGGU | nucleotide +12 | |
| | | UGGGCAGCAUUG | position | |
| | | GGAUACAGUGUG | 5/5 symmetric loop at | |
| | | AAAA | +18 position | |
| | | | 2/2 symmetric bulge | |
| | | | at −12 position | |
| | | | 6/6 symmetric loop at | |
| | | | −19 position | |
| SEQ | LRRK2_EIE | AAGGACGGGUCG | 2/2 bulge at −1 to 0 | UGUAUCCCAAUG |
| ID | | UACUGAGCAAUG | positions | CUGCCAUCAUUG |
| NO: | | CCCUAGUCAAAG | 0/9 asymmetric loop | CAAAGAUUGCUG |
| 50 | | UGGACAGCAAUC | at −6 position | ACUACAGCAUUG |
| | | UUUGCAACGAUG | 1/1 mismatch at −21 | CUCAGUAC (SEQ |
| | | GCAGCAUCGGGA | position | ID NO: 54) |
| | | UACACCUGUGAC | 1/1 mismatch at −33 | |
| | | UAA | position | |
| | | | 1/2 asymmetric bulge | |
| | | | at −42 position | |
| SEQ | LRRK2_ | GUGCCCUCUGAU | A/C mismatch at 0 | (SEQ ID NO: 54) |
| ID | FlipIntGluR2 | GUUCUUAUCCCC | position | |
| NO: | | AUUCCACAGCAG | 1/1 mimatch at +21 | |
| 51 | | UACUGAGCAAUG | position | |
| | | CCGUAGUCACACC | 1/1 mismatch at+34 | |
| | | CUAUGAUAUUGU | position | |
| | | UGUAAAUCGUAUA | GluR2 Recruiting | |
| | | ACAAUAUGAUAAG | domain at −7 position | |
| | | GUGGCAAUCUUC | 1/1 mismatch at −16 | |
| | | GCAAUGAUGGCA | position | |
| | | GCAUUGGGACAC | 1/1 mismatch at −38 | |
| | | AGUGUGAAAA | position | |
| SEQ | LRRK2_IntGluR2 | GUGCCCUCUGAU | A/C mismatch at 0 | (SEQ ID NO: 54) |
| ID | | GUUCUUAUCCCC | position | |
| NO: | | AUUCCACAGCAG | 1/1 mimatch at +21 | |
| 52 | | UACUGAGCAAUG | position | |
| | | CCGUAGUCAGUG | 1/1 mismatch at +34 | |
| | | GAAUAGUAUAACA | position | |
| | | AUAUGCUAAAUGU | GluR2 Recruiting | |
| | | UGUUAUAGUAUCC | domain at −7 position | |
| | | CACGCAAUCUUC | 1/1 mismatch at −16 | |
| | | GCAAUGAUGGCA | position | |
| | | GCAUUGGGAUAC | | |
| | | AGUGUGAAAA | | |

### Example 6

### Introduction of IVT Guide RNA into Cells Containing the G2019S LRRK2 Mutation

EBV transformed B cells (LRRK2 G2019S patient-derived lymphoblastoid cell lines; LCLs) encoding one mutant allele of the G2019S mutation in LRRK2 (heterozygous) from a donor were procured and used throughout these experiments to assess ADAR-mediated RNA editing from A to G and reversion to the wild-type LRRK2 allele.

7 IVT generated engineered guide RNAs (from EXAMPLE 5) were tested against LRRK2 as well as 1 IVT engineered guide RNA against RAB7A (as a control). All engineered guide RNAs were nucleofected in LCL cells using the Lonza X nucleofector with program EH100. Reaction conditions included approximately 40 nmol or 60 nmol of each IVT engineered guide RNA and about 2×10\hat{ }5 LCL cells per reaction. The reaction was split into 2 wells each containing 1×10\hat{ }5 cells and cells collected for RNA isolation at either 3 hours or 7 hours. At collection, cells were spun at 1,500×g for 1 min, media was then removed, and 180 μl of RLT lysis buffer+beta-mercaptoethanol (BMe) was added to each well. A Qiagen RNeasy protocol and kit were used to isolate RNA, followed by a New England Biolabs (NEB) ProtoScript II First-Strand cDNA synthesis kit.

LRRK2 mRNA specific primers, outside of the target regions, were used to amplify the region that the IVT engineered guide RNAs were targeting (TABLE 16). The primers had no sequence overlap with any of the engineered guide RNAs. LRRK2 primers 1 and 2 were used to amplify the mRNA, and primer 4 was used for sequencing of the target region. Sanger traces were analyzed to assess editing efficiency of each IVT guide.

TABLE 16
LRRK2 mRNA specific primers

| SEQ ID NO | Primer Name | Sequence |
|---|---|---|
| SEQ ID NO: 55 | LRRK2 1 | CGTAGCTGATGGTTTGAGATACCT |
| SEQ ID NO: 56 | LRRK2 2 | ACCAAATGAATAAACATCAGCCTGTTG |
| SEQ ID NO: 57 | LRRK2 4 | TTTCCTCTGGCAACTTCAGGTG |

### Example 7

### LRRK2 Targeting Guide RNAs for Correction of the G2019S Mutation and Treatment of Parkinson's Disease

This example describes treatment of Parkinson's disease in patients having the G2019S mutation in LRRK2 using the guide RNAs of the present disclosure. Parkinson patients diagnosed with the G2019S mutation are administered any of the guide RNAs described herein (e.g., those listed in TABLE 15). Guide RNAs are prepared, for example, by PCR and IVT (as described in EXAMPLE 6 and EXAMPLE 7) or are genetically encoded in a DNA construct encapsidated in an AAV. Guide RNAs are administered to a subject by any route of administration disclosed herein, such as intravenous injection, intracerebroventricular, intraparenchymal, intracisternal, or intrathecal injection. The subject is a human or non-human animal.

For genetically encoded guide RNAs, the coding sequence of the guide RNA (e.g., such as those listed in TABLE 12) with their T7 promoter sequence replaced with a U7, a U1, a U6, an H1, or a 7SK promoter sequence, is cloned into a viral vector, such as an adenoviral vector, an adeno-associated viral vector (AAV), a lentiviral vector, or a retroviral vector. Alternatively, the coding sequence of the guide RNA (e.g., such as those listed in TABLE 12) with their T7 promoter sequence replaced with a U7, a U1, a U6, an H1, or a 7SK promoter sequence is prepared by PCR or gBlocks™ Gene Fragments and the coding sequence is formulated in a pharmaceutical formulation, a nanoparticle, or a dendrimer (e.g., via encapsulation or direct attachment).

RNA editing is monitored as follows: ˜1×10\hat{ }5 cells are collected for RNA isolation after a week. At collection, cells are spun at 1,500×g for 1 min. The media is removed. 180 ul of RLT buffer+BMe is added to each well. RNA is isolated from the cells and cDNA is synthesized and sequenced (e.g., via Sanger sequencing or NGS). Percent on-target editing, percent off-targeting editing, or a combination of both is quantified. In particular, the ability of each guide RNA to facilitate ADAR-mediated the RNA editing of the A to G LRRK2 to correct the G2019S mutation is calculated (e.g., by quantitating the difference of trace signal of the LRRK2 mRNA with a G (edited) and an A (unedited) at the 6055th nucleotide).

### Example 8

### Multiplexed Targeting of LRRK2 and SNCA Using Engineered Guide RNAs

Because polymorphisms in either LRRK2 (G2019S) or SNCA may be associated with increased risk of idiopathic Parkinson's Disease, simultaneous manipulation of the expression of the two genes can be a useful treatment. RNA editing, as illustrated in the current disclosure, is modular; the RNA editing enzyme and the RNA targeting guide are two different entities. Therefore, engineered guide RNAs can be multiplexed to achieve simultaneous correction of more than one distinct targets. For example, to treat idiopathic Parkinson's Disease patients with contributing polymorphisms in LRRK2 (G2019S) and SNCA, two engineered guide RNAs are designed. The first engineered guide RNA is selected from any of the LRRK2-targeting engineered guide RNAs disclosed herein (e.g., those disclosed in TABLE 15) and targets the LRRK2 G2019S mutation for ADAR-mediated editing of an A to a G at the 6055^{th} nucleotide (e.g., see EXAMPLE 7). The second engineered guide RNA is selected from any of the SNCA-targeting engineered guide RNAs disclosed herein and targets the ATG start codon of SNCA for ADAR-mediated editing of an A to a G. Upon editing of the start site, expression of the alpha-synuclein protein is decreased. Expression of each of these engineered guide RNAs can be independently or together driven under an upstream U7, U1, U6, H1, 7SK promoter and cloned into a single viral vector or two separate viral vectors, such as an adenoviral vector, an adeno-associated viral vector (AAV), a lentiviral vector, or a retroviral vector. Guide RNAs are administered to a subject by any route of administration disclosed herein, such as intravenous injection, intracerebroventricular, intraparenchymal, intracisternal, or intrathecal injection. The subject is a human or non-human animal.

In particular, the ability of each guide RNA to facilitate ADAR-mediated the RNA editing of the A to G LRRK2 to correct the N2081D mutation is calculated (e.g., by quantitating the difference of trace signal of the LRRK2 mRNA with a G (edited) and an A (unedited) at the 6055th nucleotide).

The expression level of SNCA is monitored as follows: knockdown of alpha-synuclein protein is assessed using Western Blot, ELISA, or Meso Scale Discovery (MSD) analysis.

### Example 9

### LRRK2 Designs with High Specificity and Efficiency

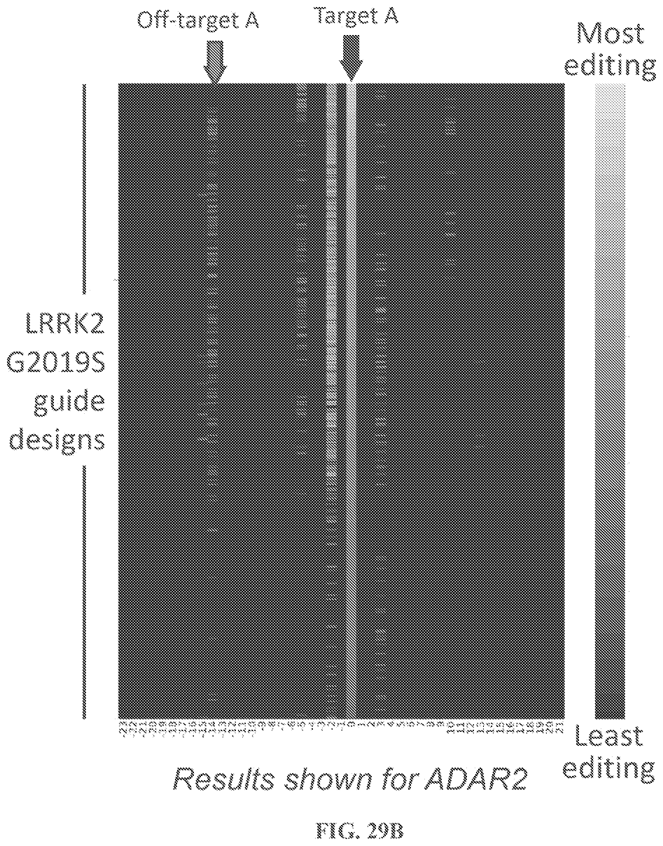

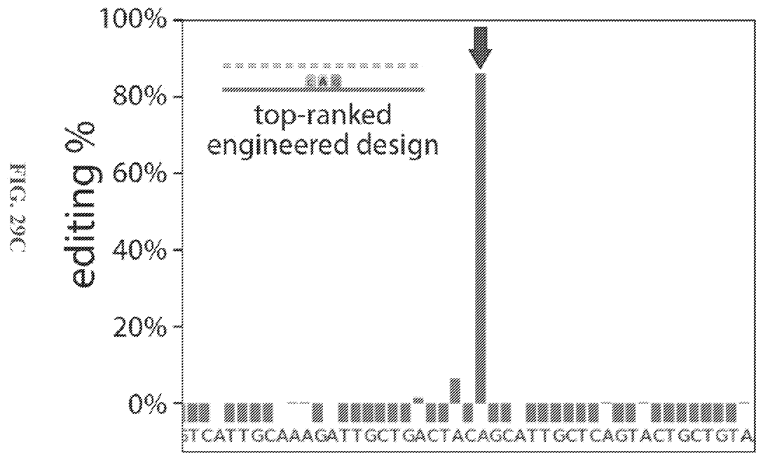

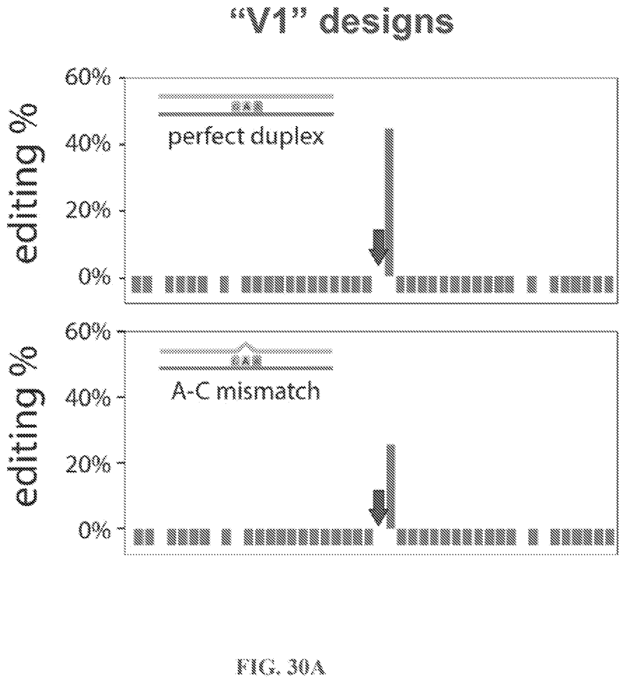

High throughput screening (HTS) of 2,540 gRNA sequences against the LRRK2*G2019S mutation identified designs with superior on-target activity and specificity. Data and results are shown in FIG. 29A to FIG. 29C. Top ranking designs are tested against LRRK2 G2019S mRNA in disease model cell lines.

This example describes engineered guide RNAs of the present disclosure targeting LRRK2 mRNA. The region of the LRRK2 mRNA that was targeted was an A at position 6055 of a LRRK2 mRNA, which encodes for a pathogenic G2019S mutant protein.

Self-annealing RNA structures comprising the engineered polynucleotide sequences of TABLE 25 (and control engineered polynucleotide sequences) and the sequences of the regions targeted by the guide RNAs were contacted with an RNA editing entity (e.g., a recombinant ADAR1 and/or ADAR2) under conditions that allow for the editing of the regions targeted by the guide RNAs. The regions targeted by the guide RNAs were subsequently assessed for editing using next generation sequencing (NGS).

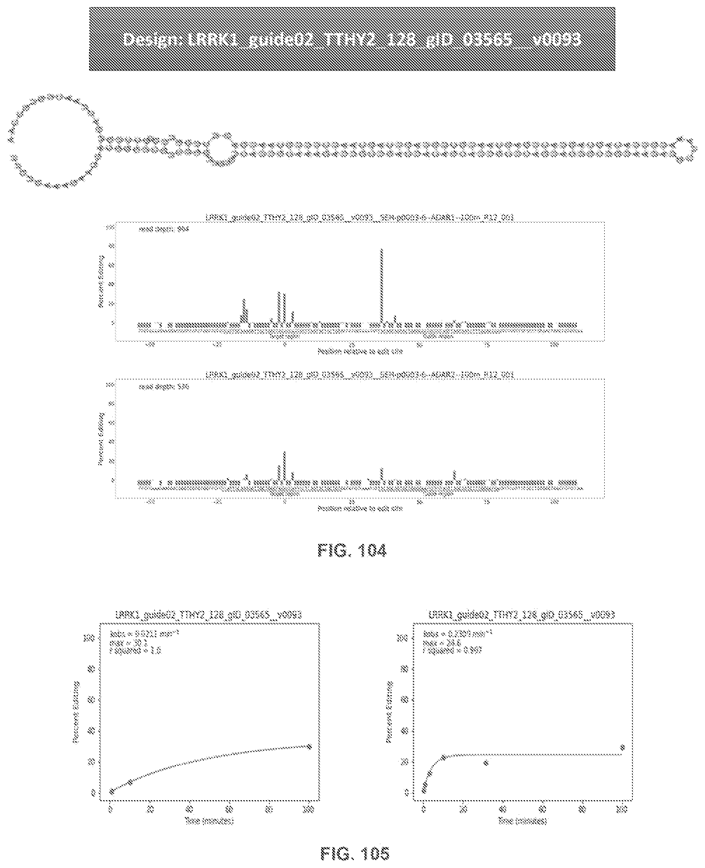

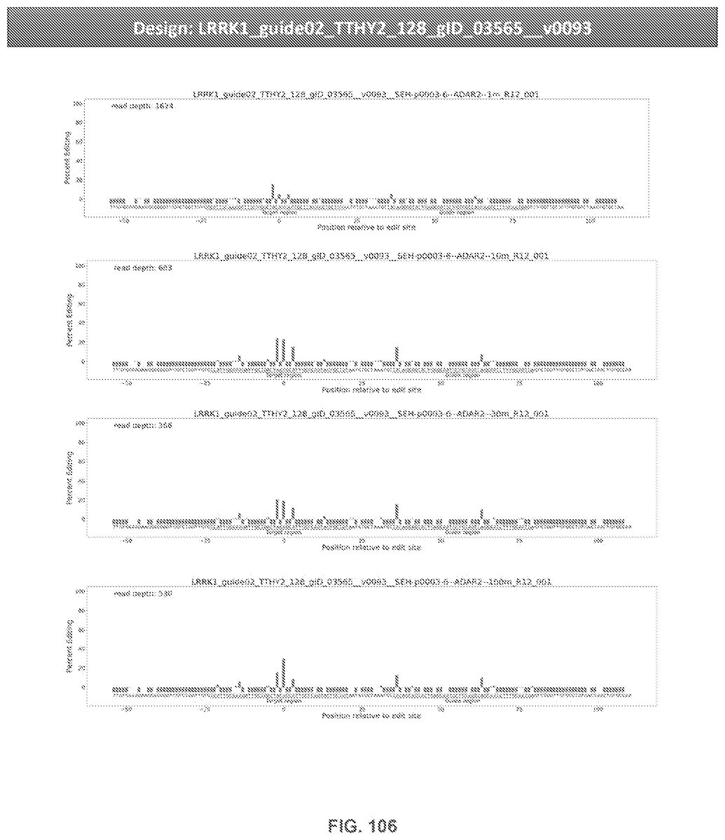

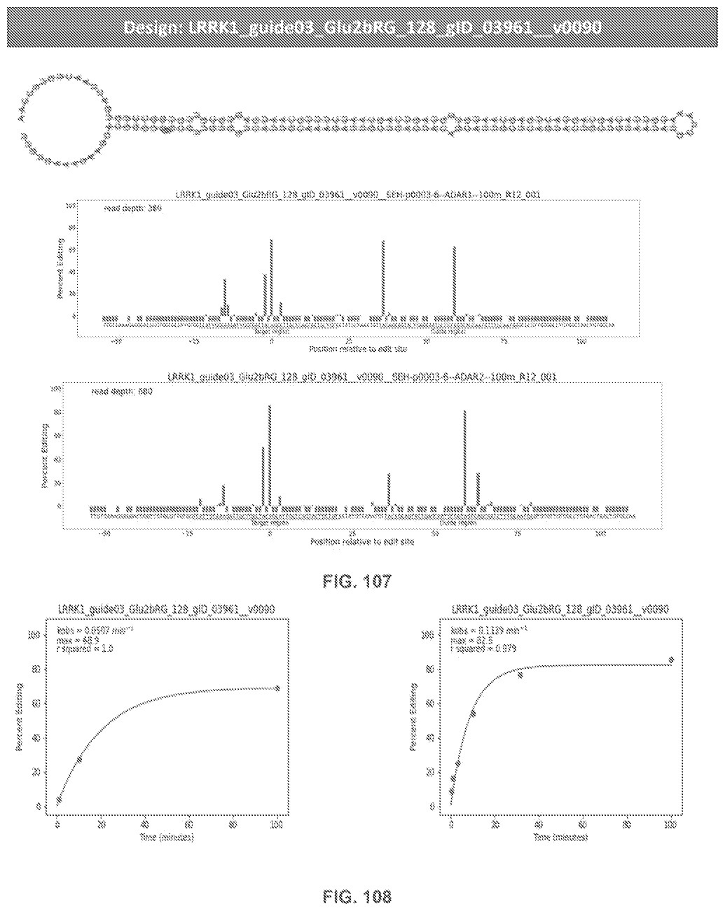

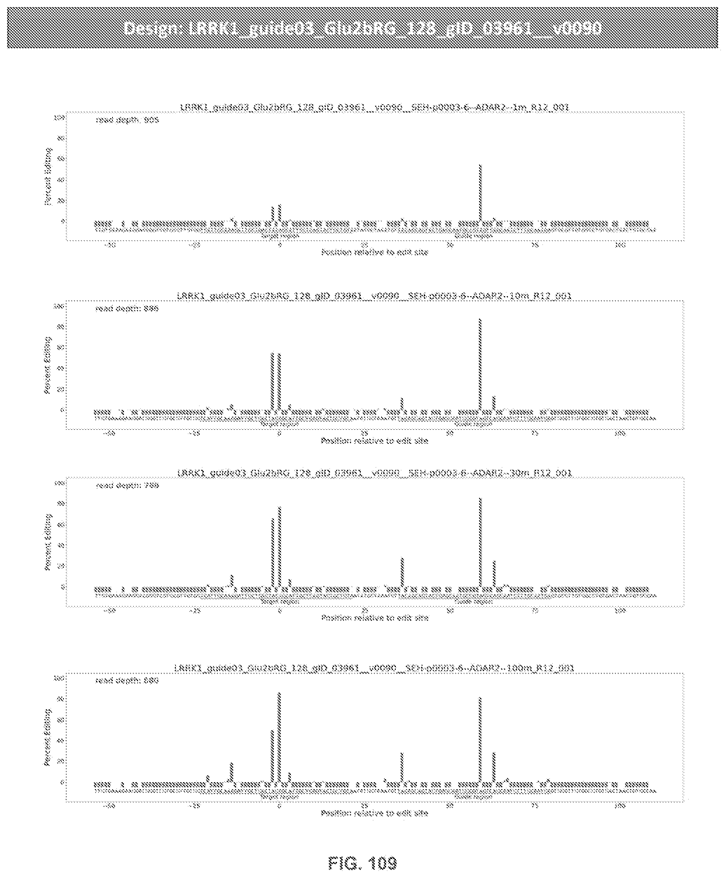

FIG. 104-FIG. 110 show control guide RNA designs for targeting LRRK2, the percentage editing as a function of time for each engineered polynucleotide as determined by sequencing, and the editing at the target A to be edited (“0” on the x-axis) and at RNA editing at off-target positions (represented as black bars at positions that are not “0”). LRRK1_guide02_TTHY2_128_gID_03565__v0093 is a guide RNA that forms a perfect duplex with the target RNA and has a sequence of 5′-TACAGCAGTACTGAGCAATGCTGTAGTCAGCAATCTTTGCAATGA-3′ (SEQ ID NO: 109). LRRK1_guide03_Glu2bRG_128_gID_03961__v0090 is a guide RNA that forms one A/C mismatch with the target RNA and has a sequence of 5′-TACAGCAGTACTGAGCAATGCCGTAGTCAGCAATCTTTGCAATGA-3′ (SEQ ID NO: 110).

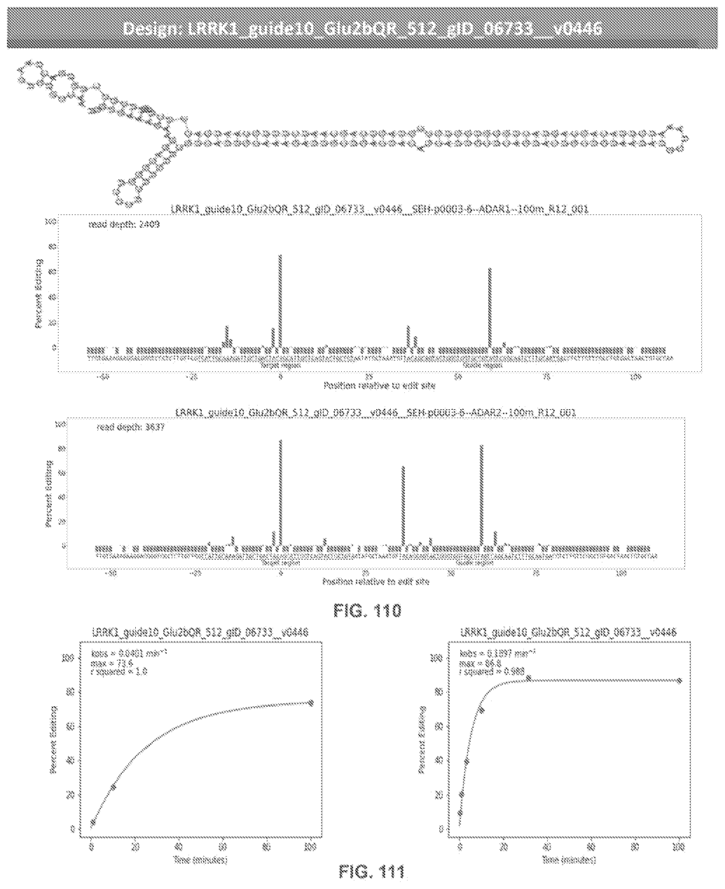

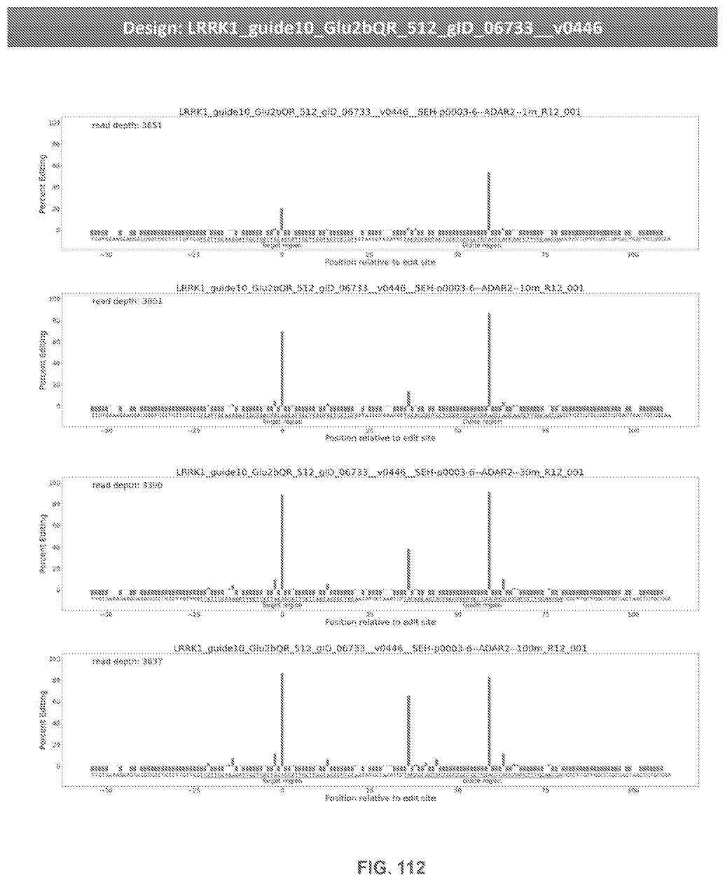

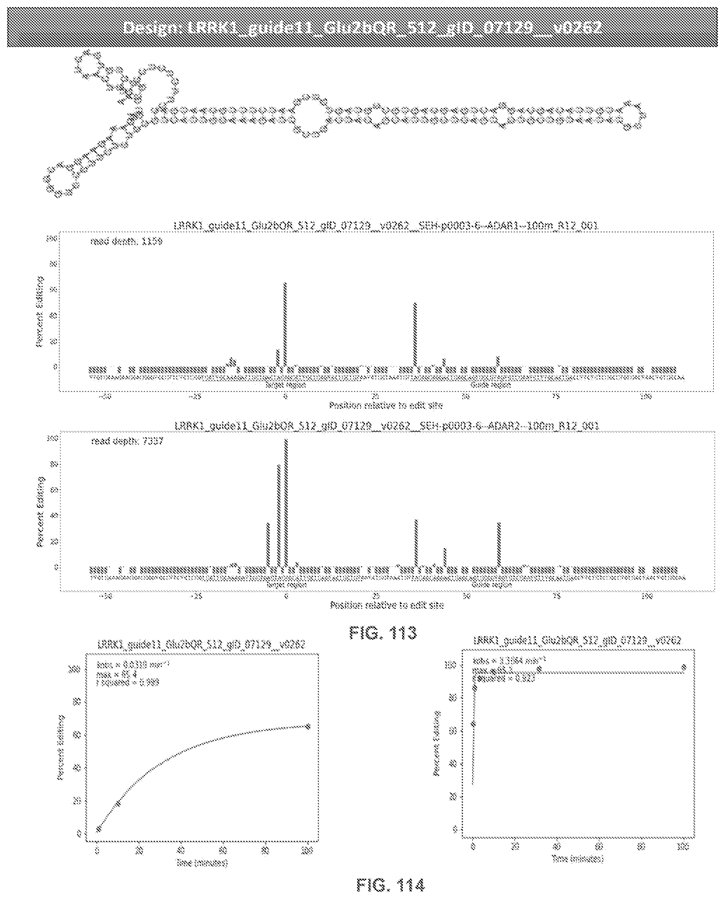

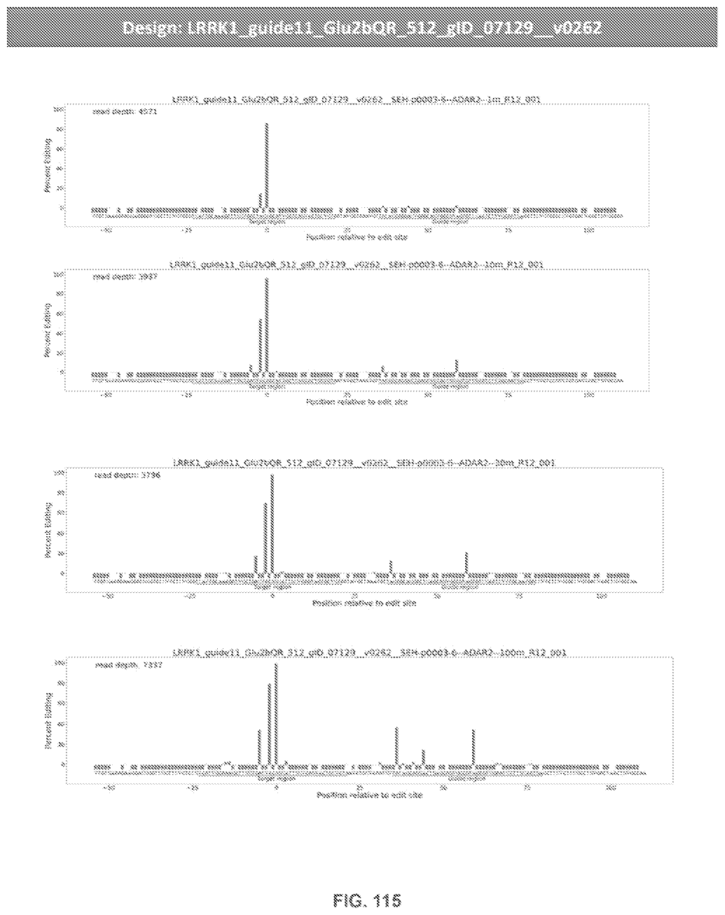

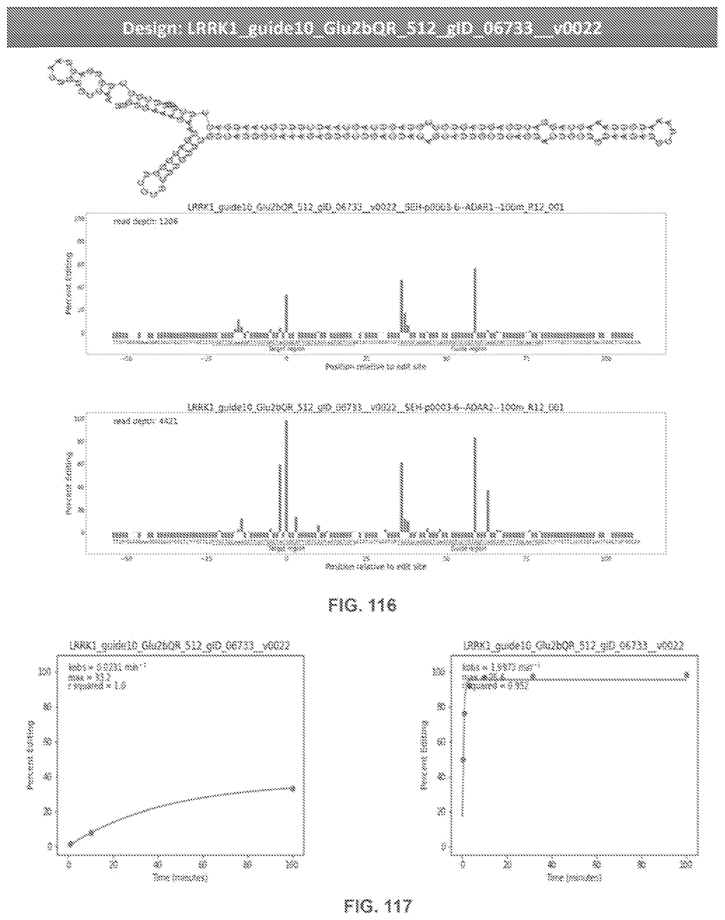

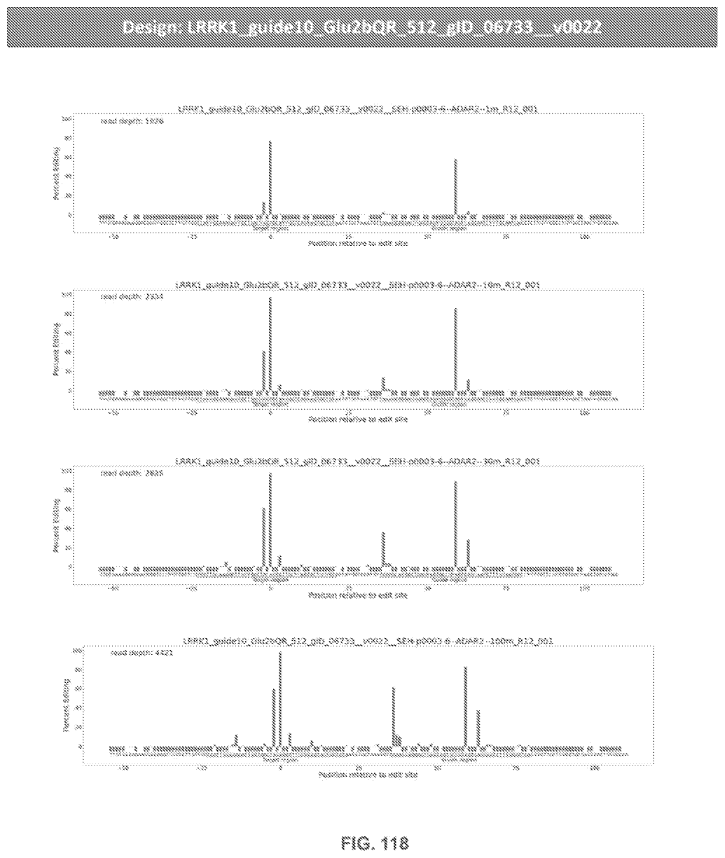

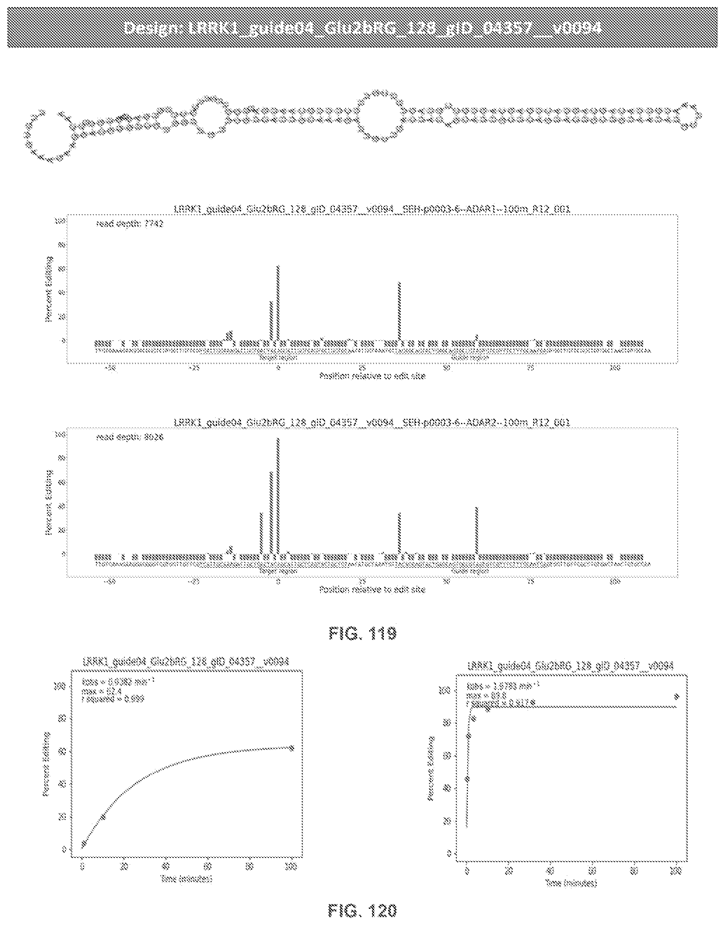

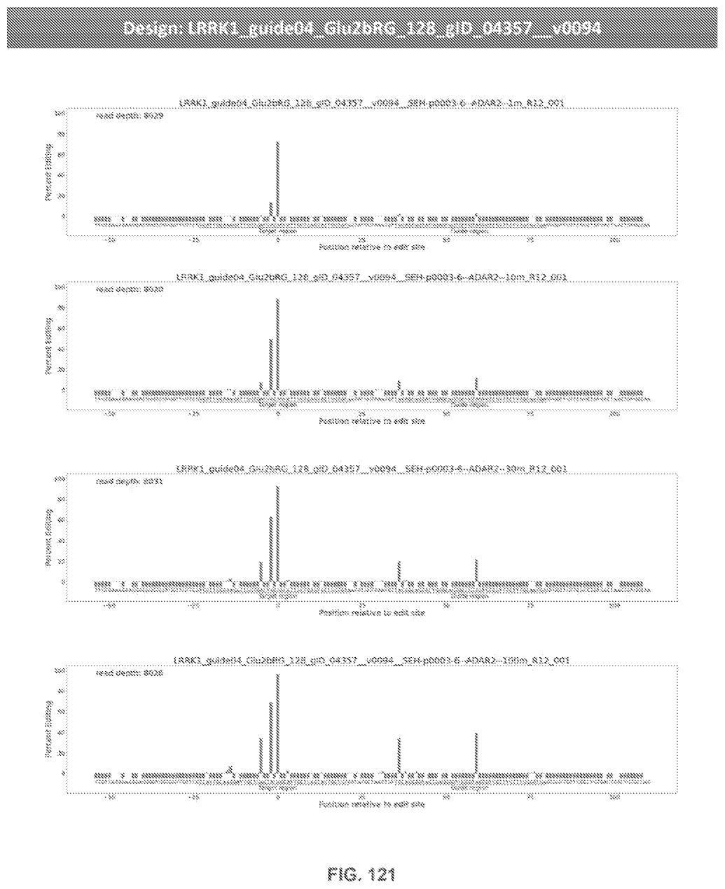

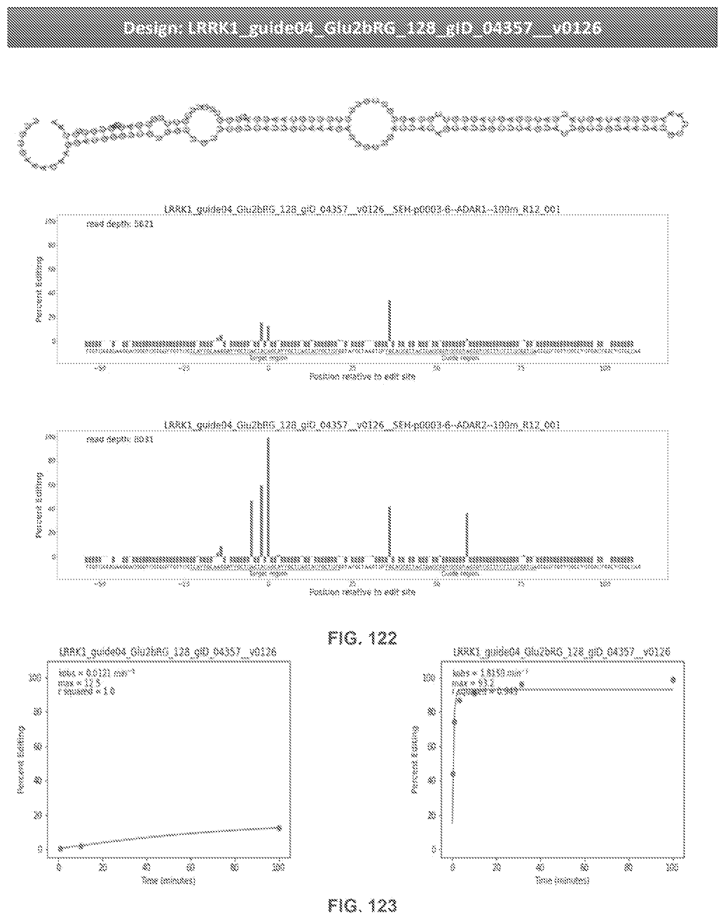

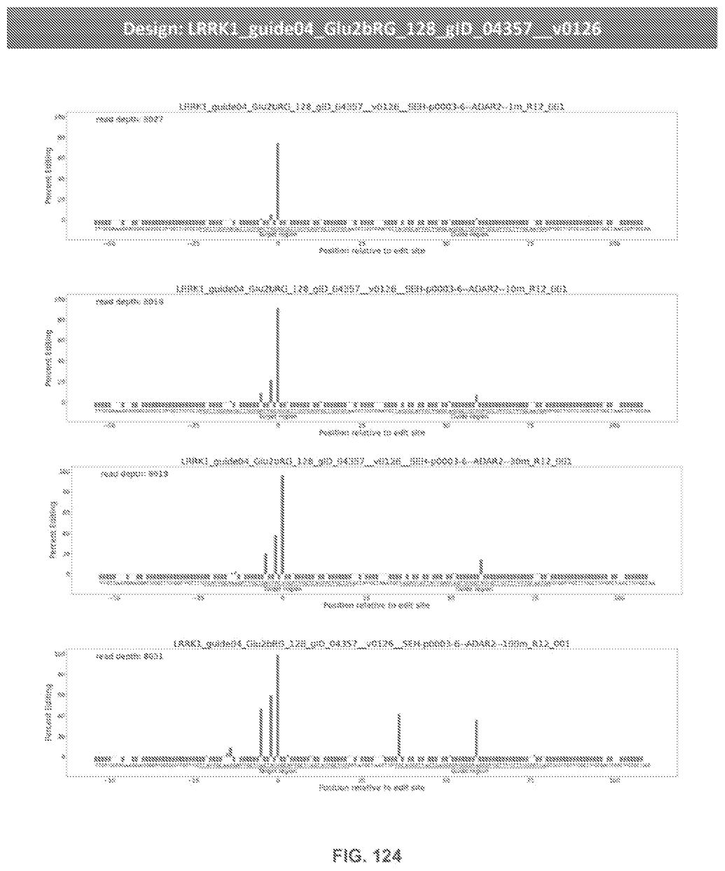

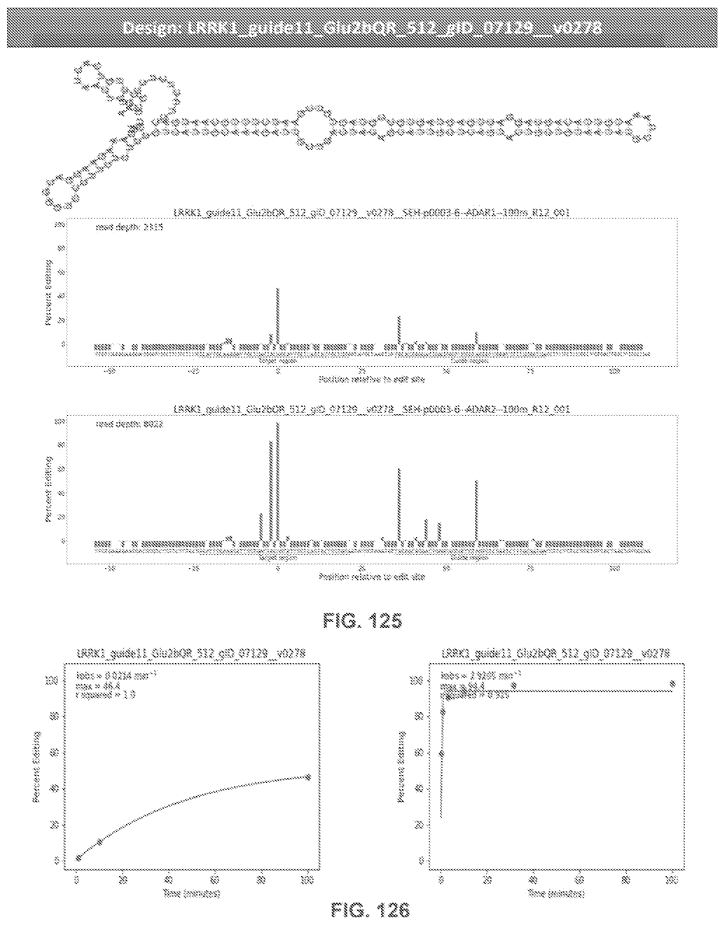

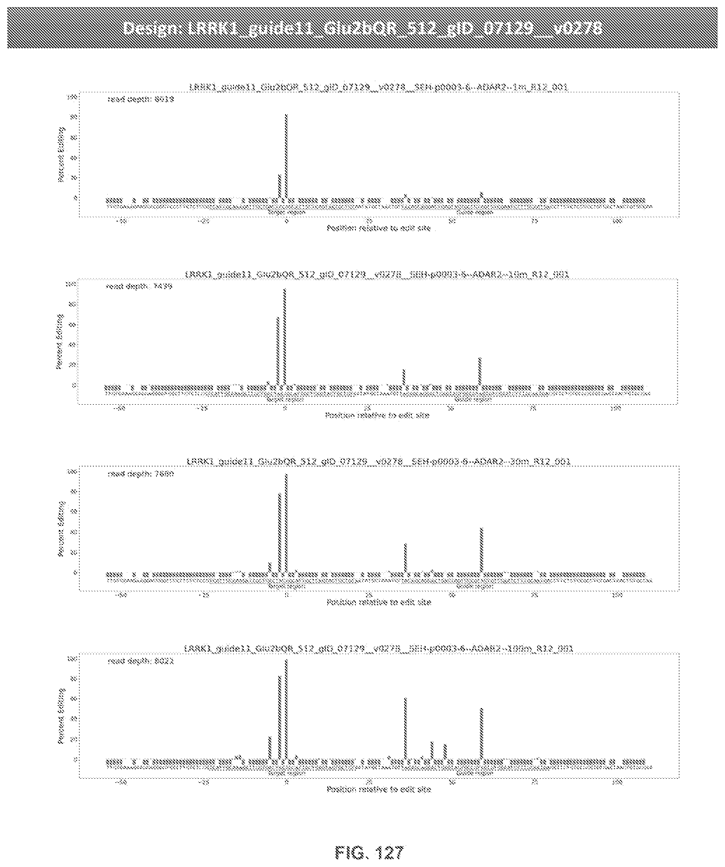

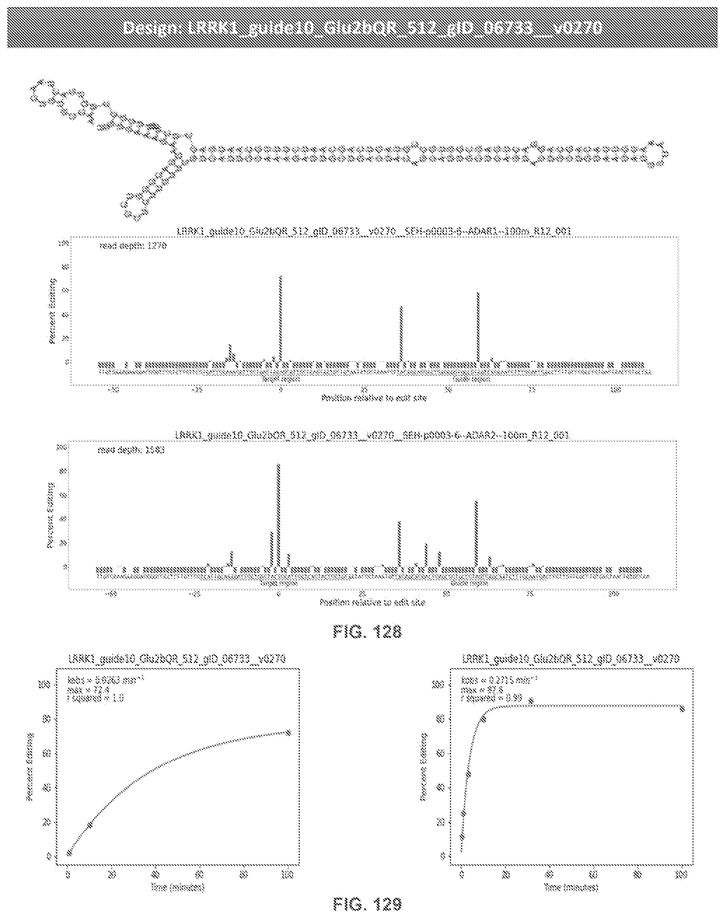

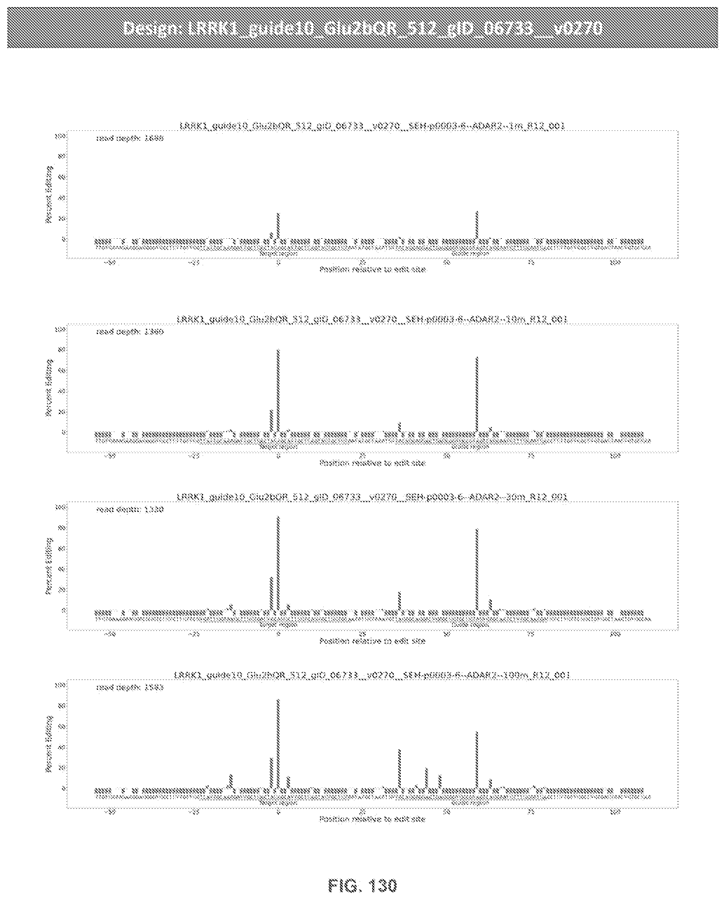

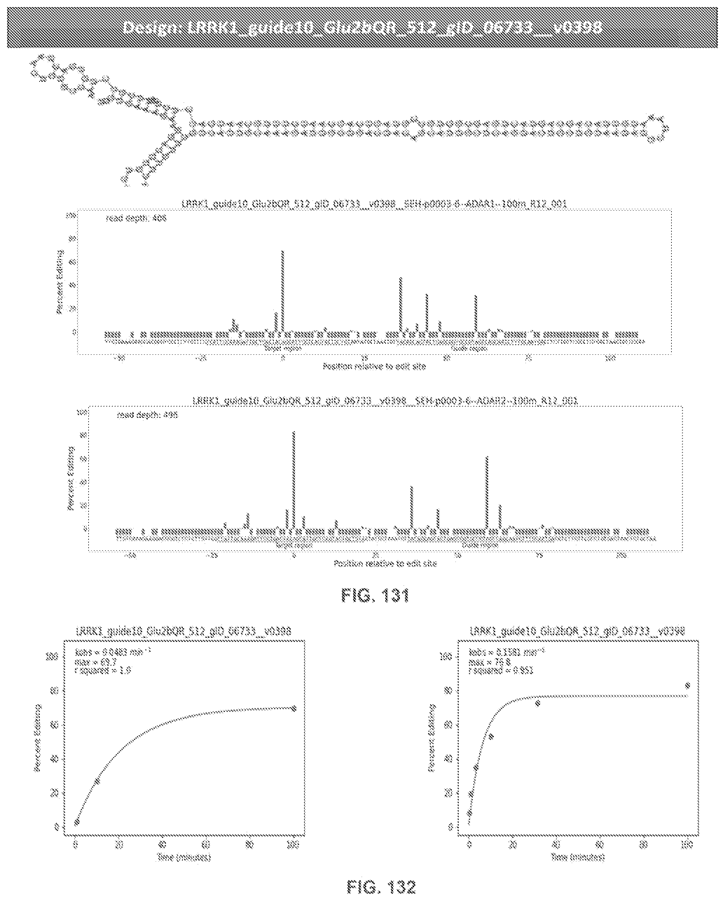

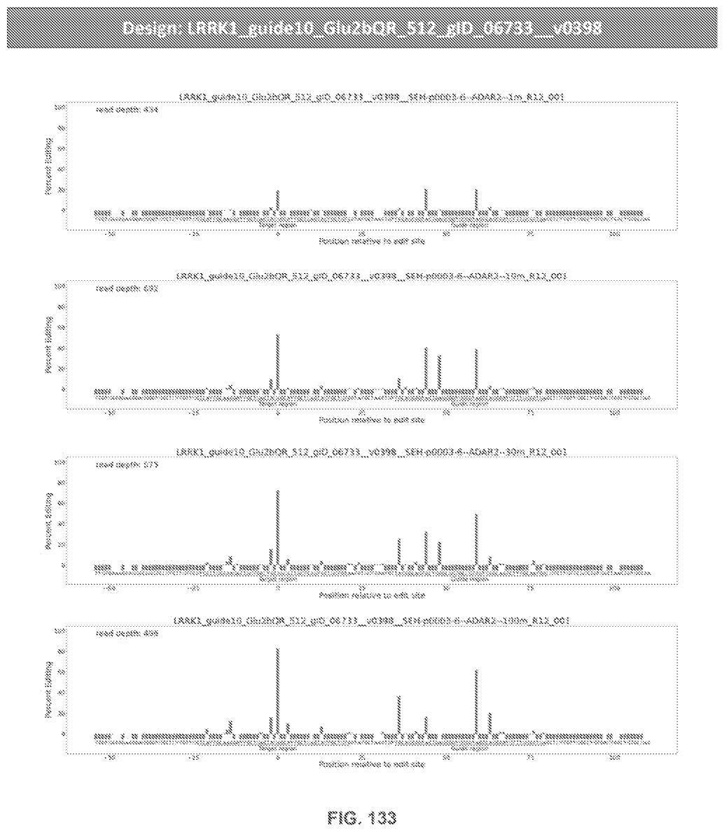

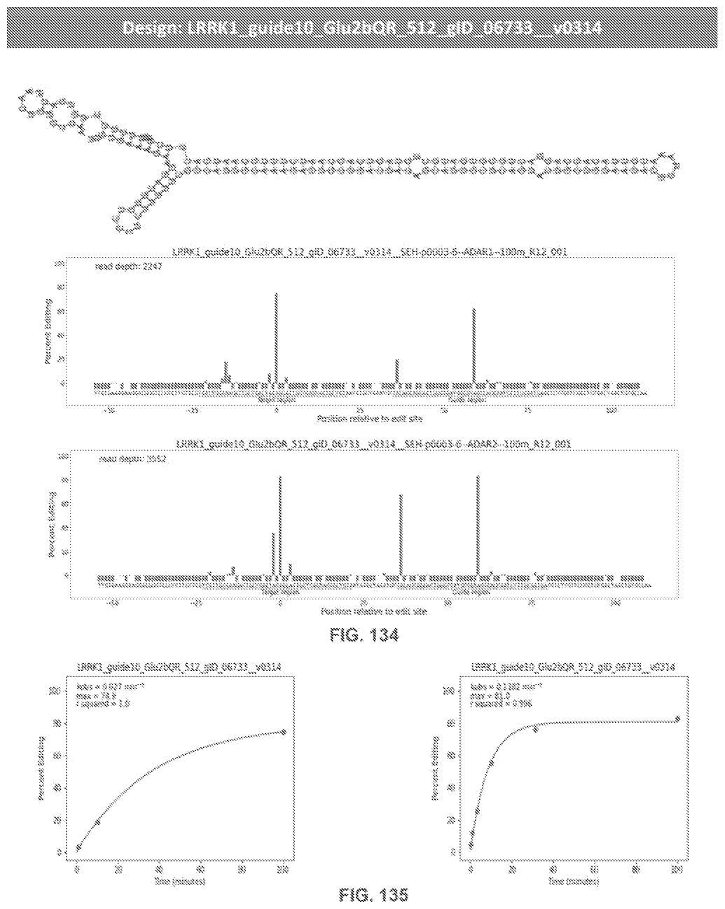

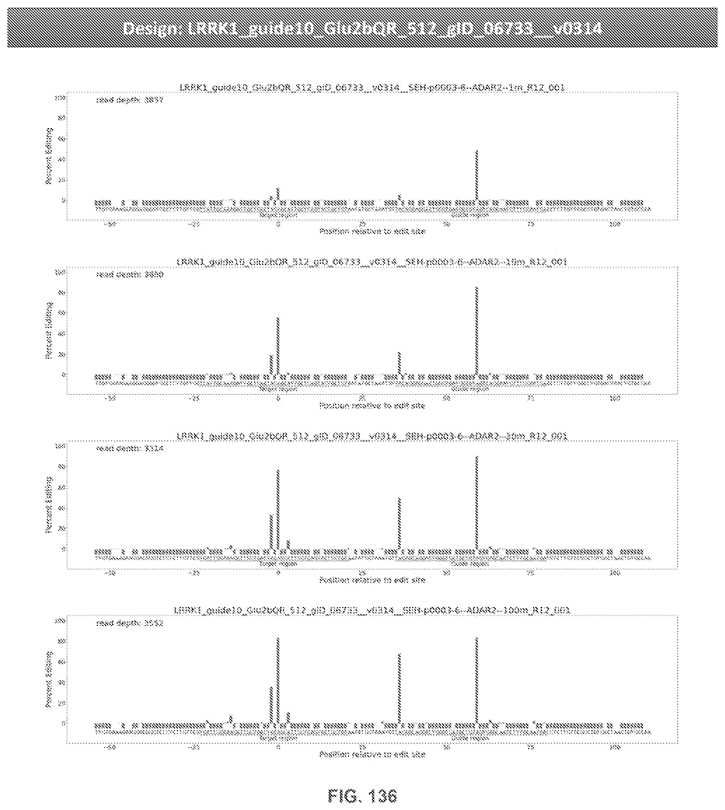

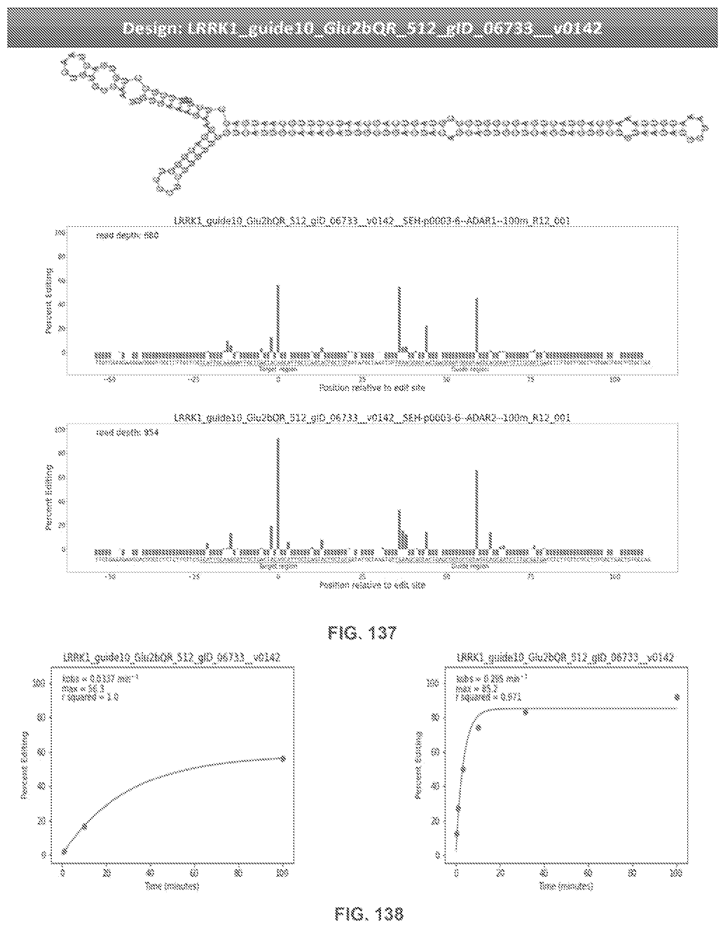

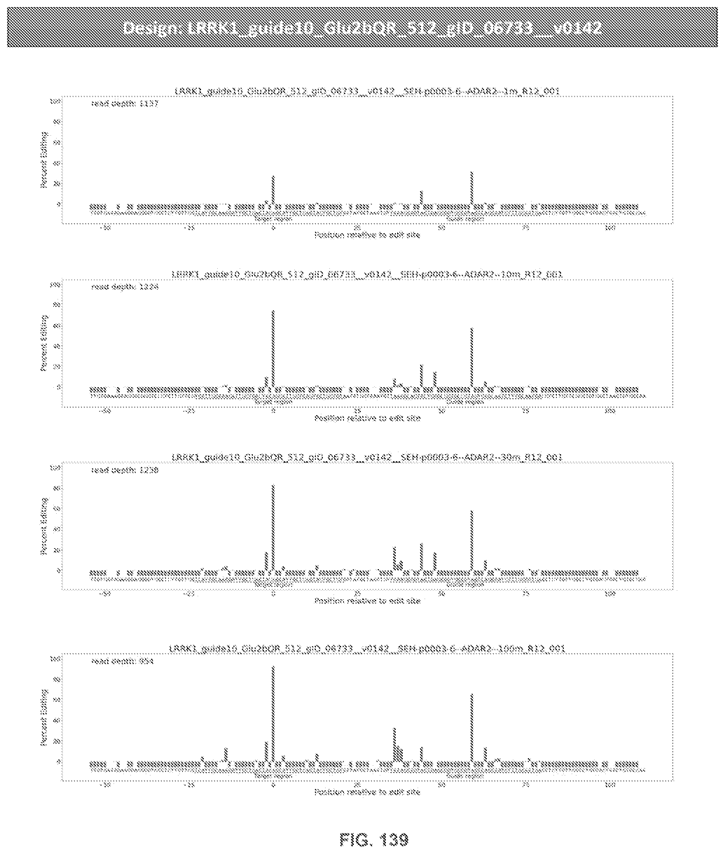

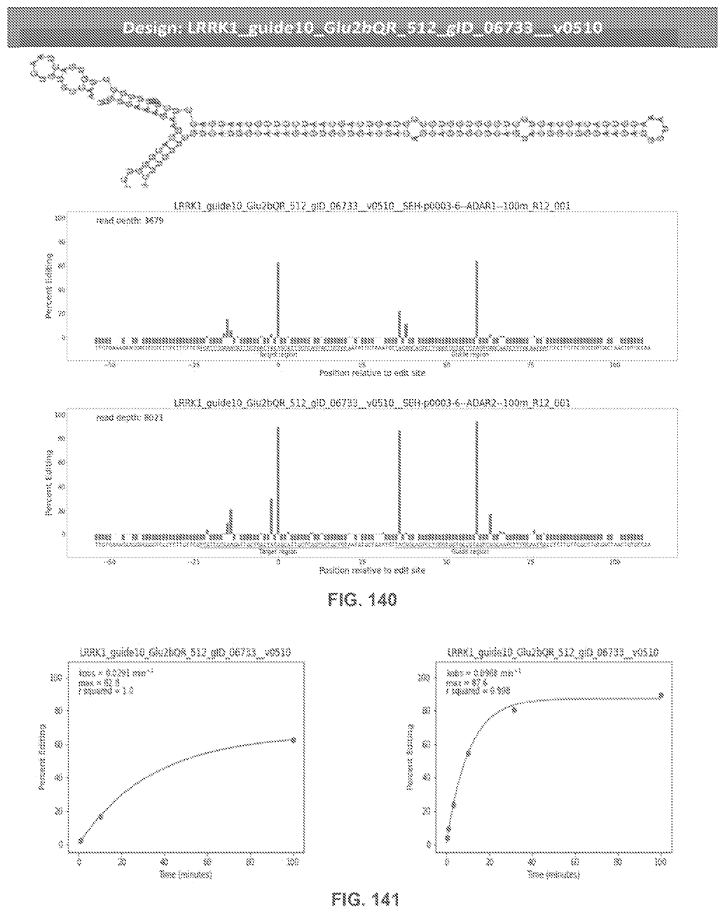

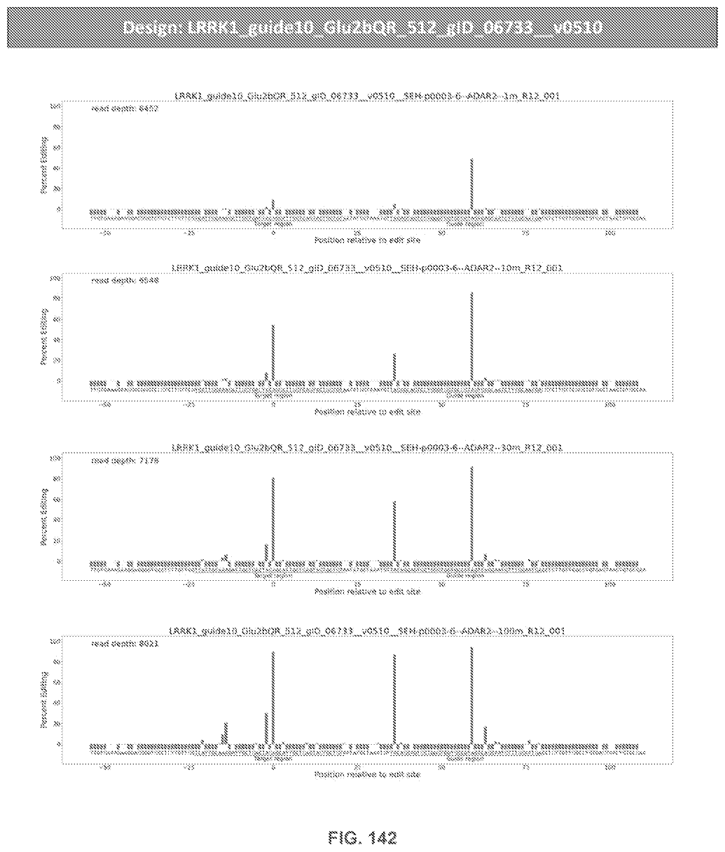

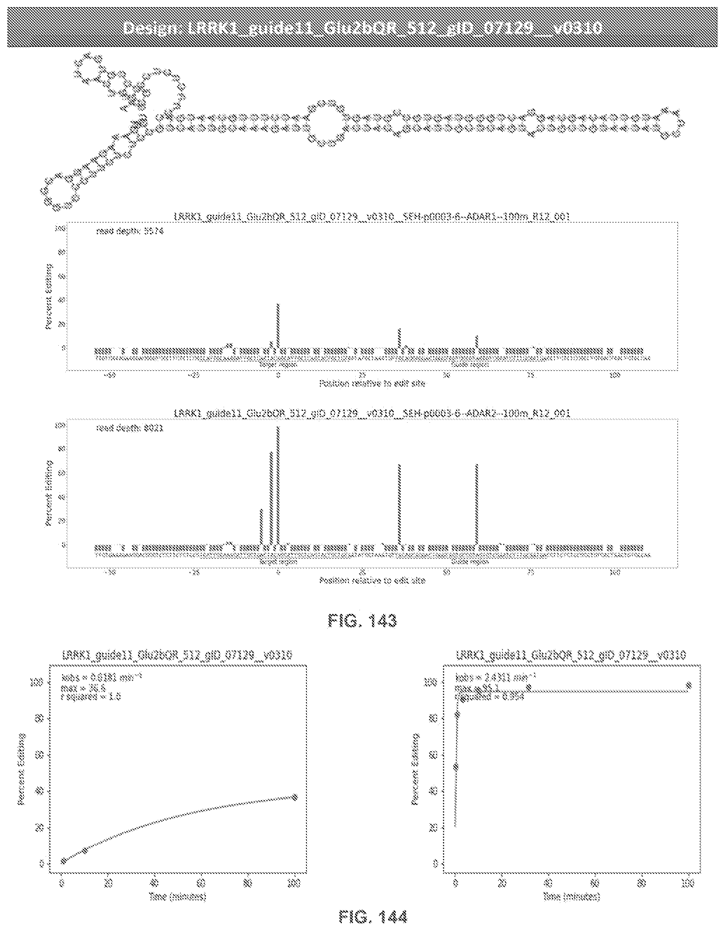

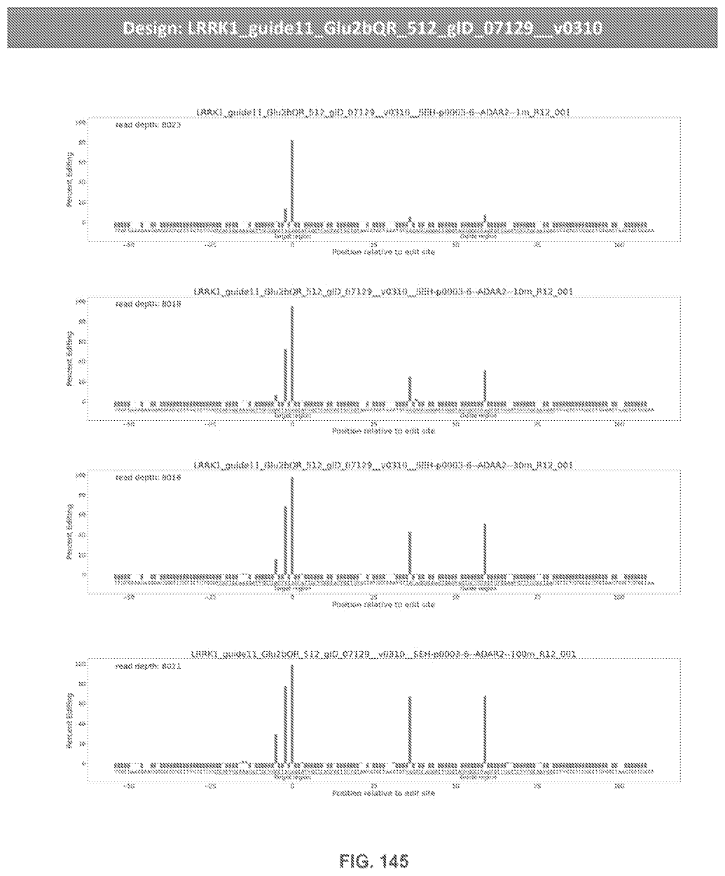

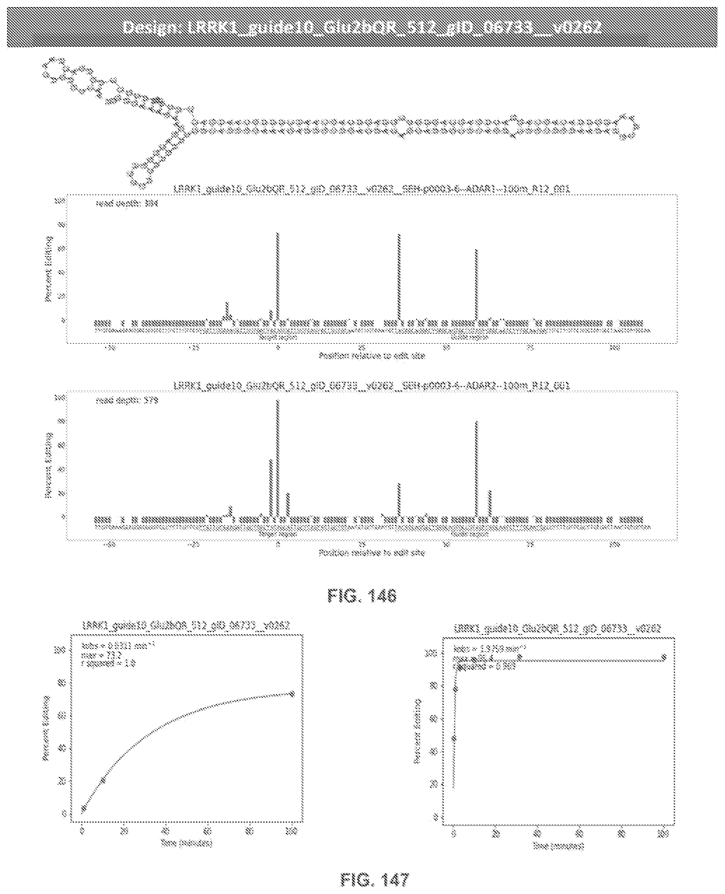

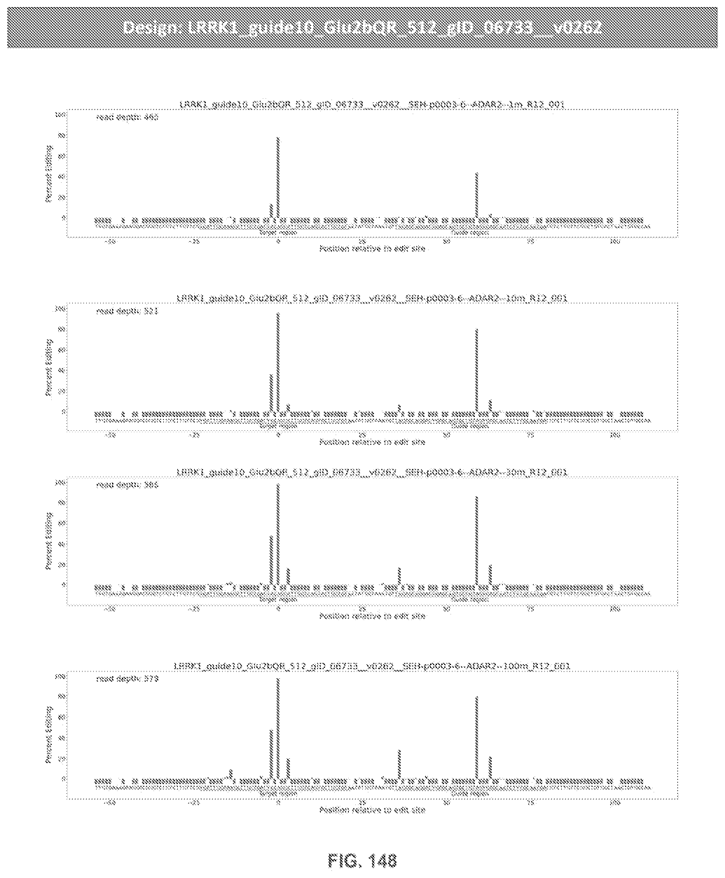

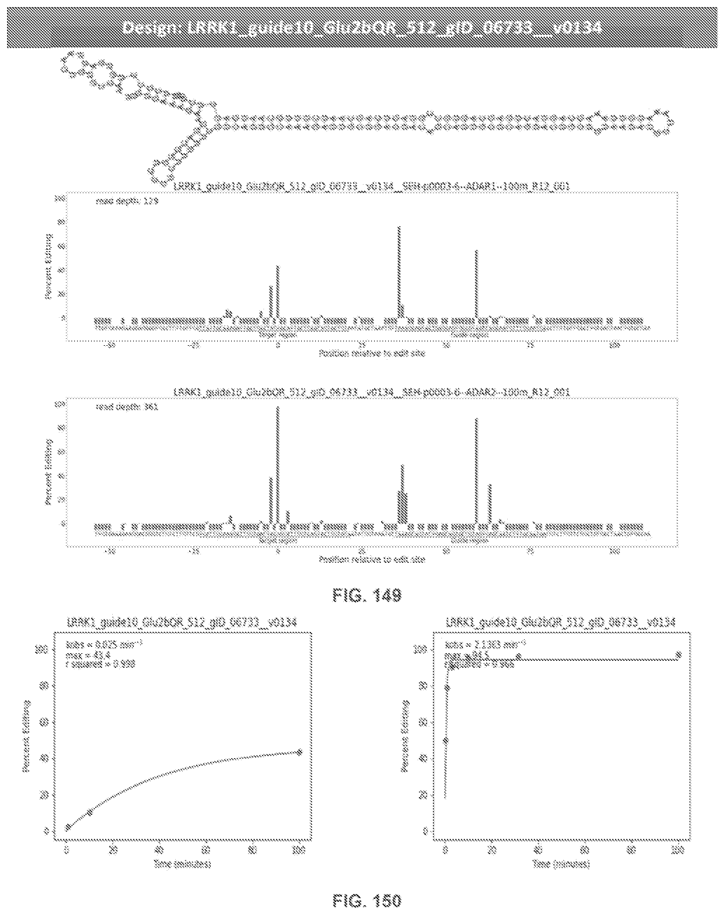

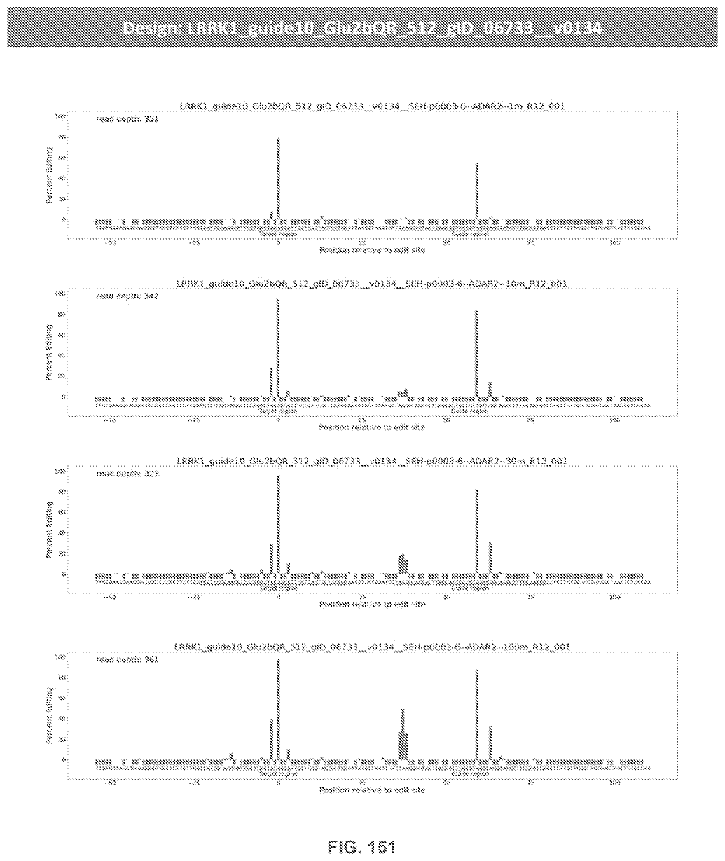

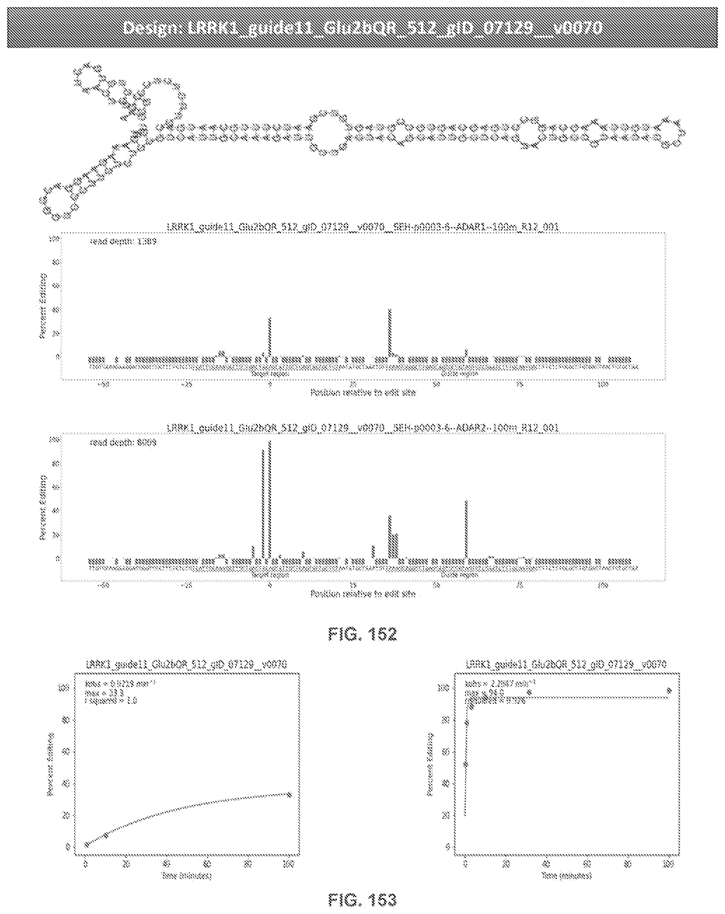

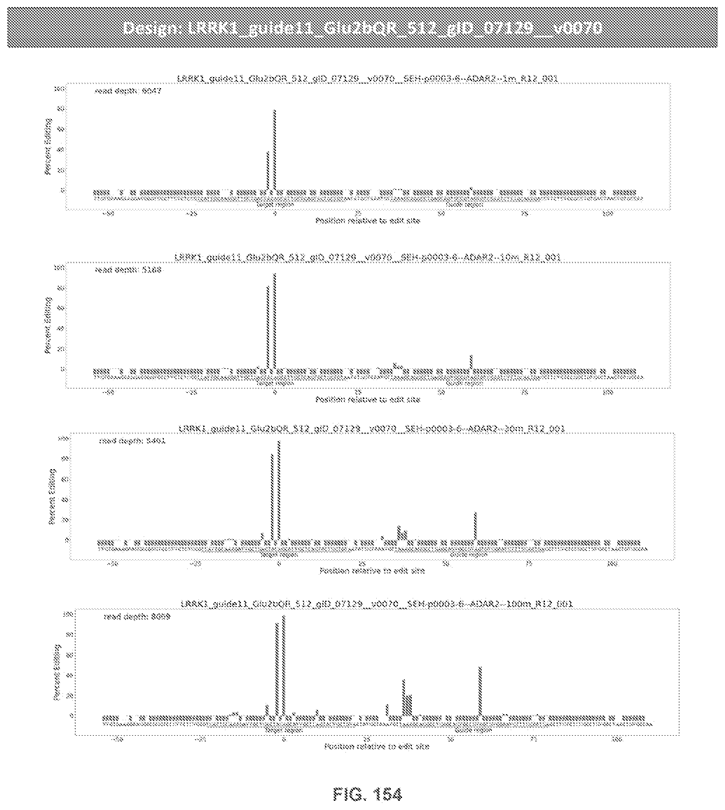

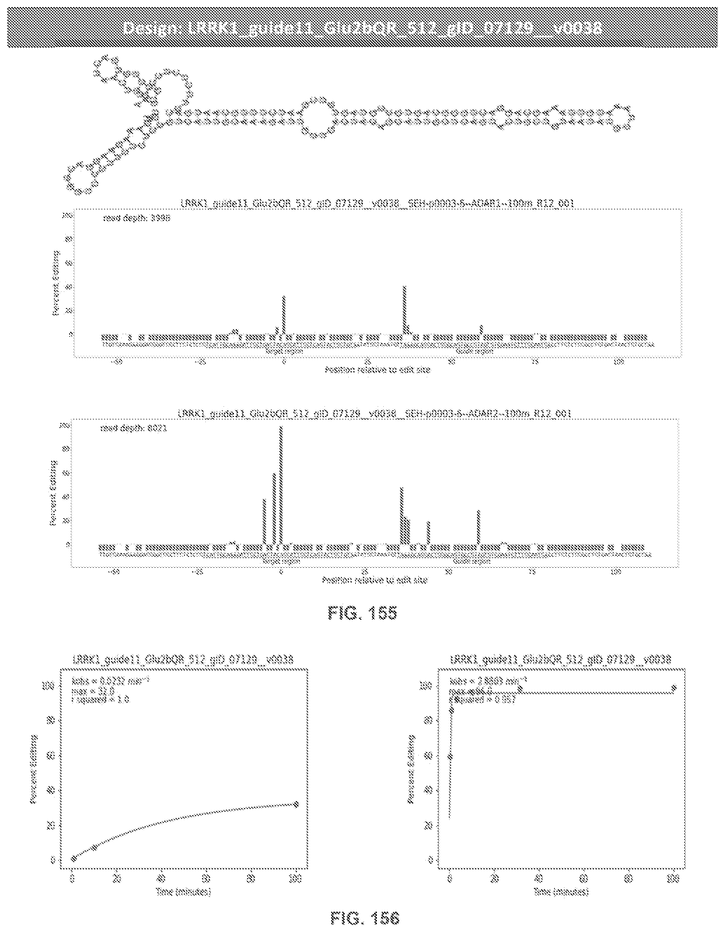

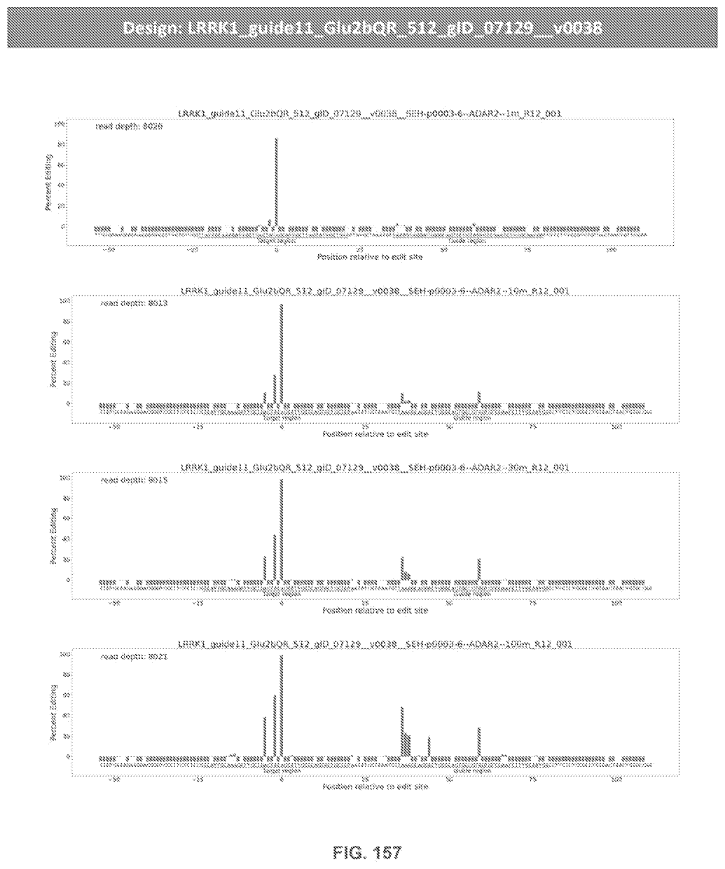

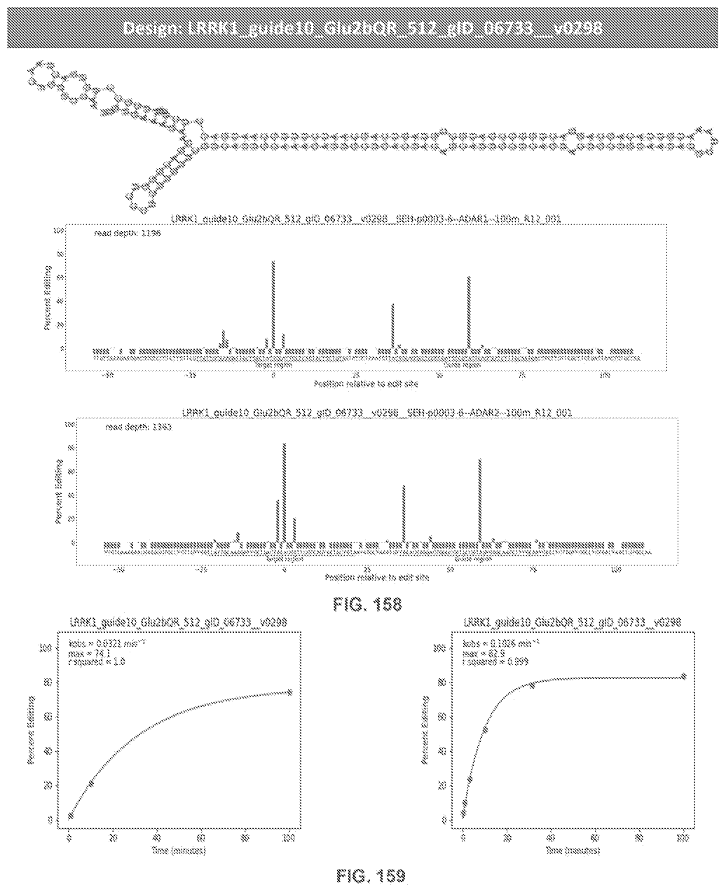

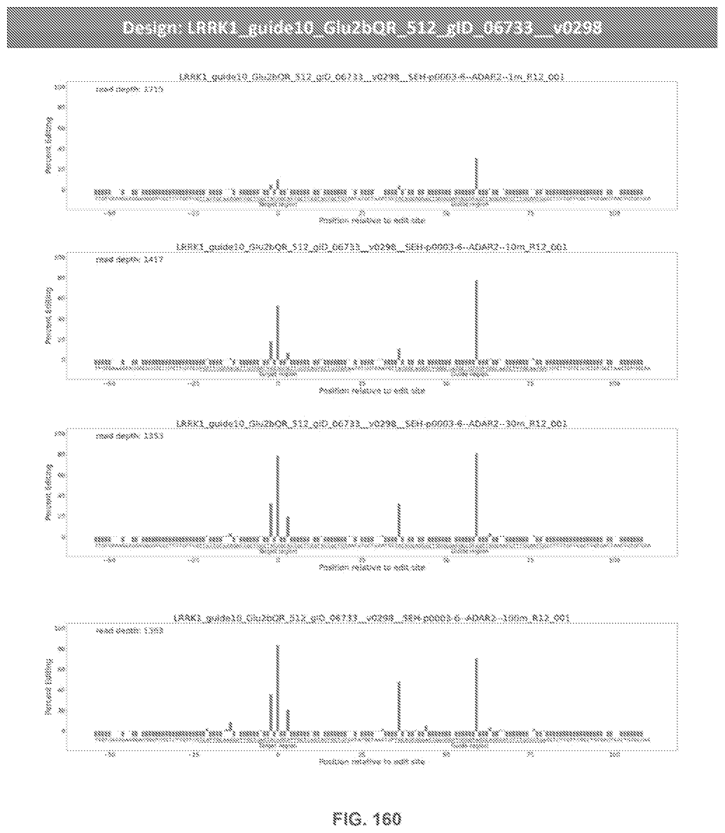

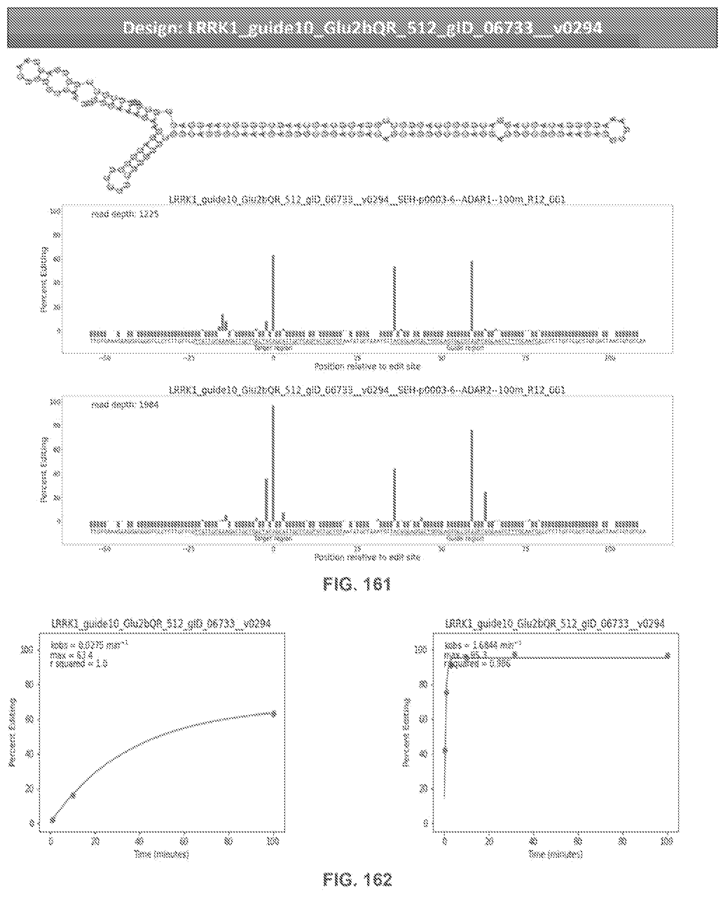

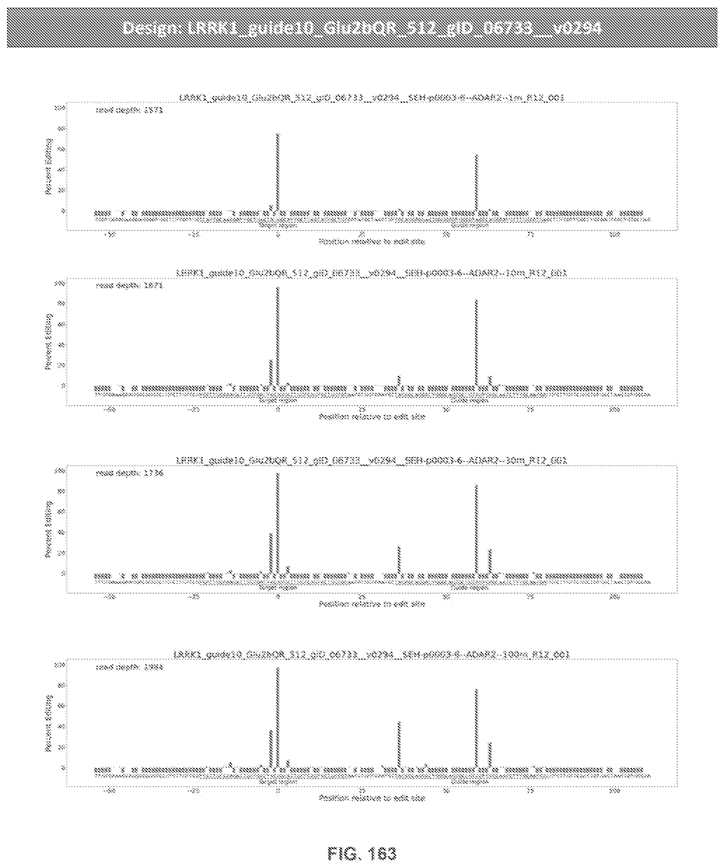

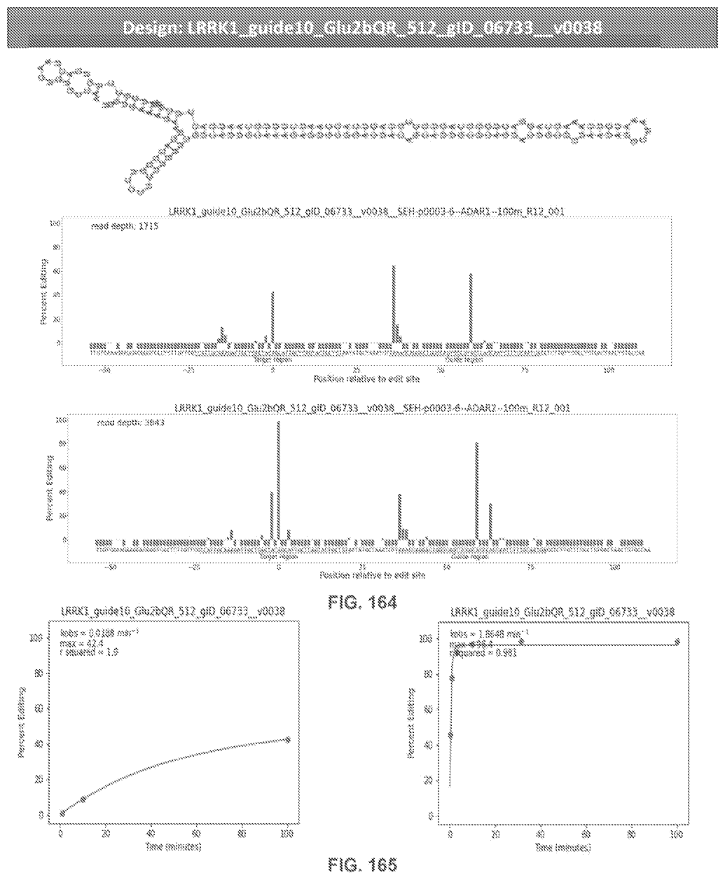

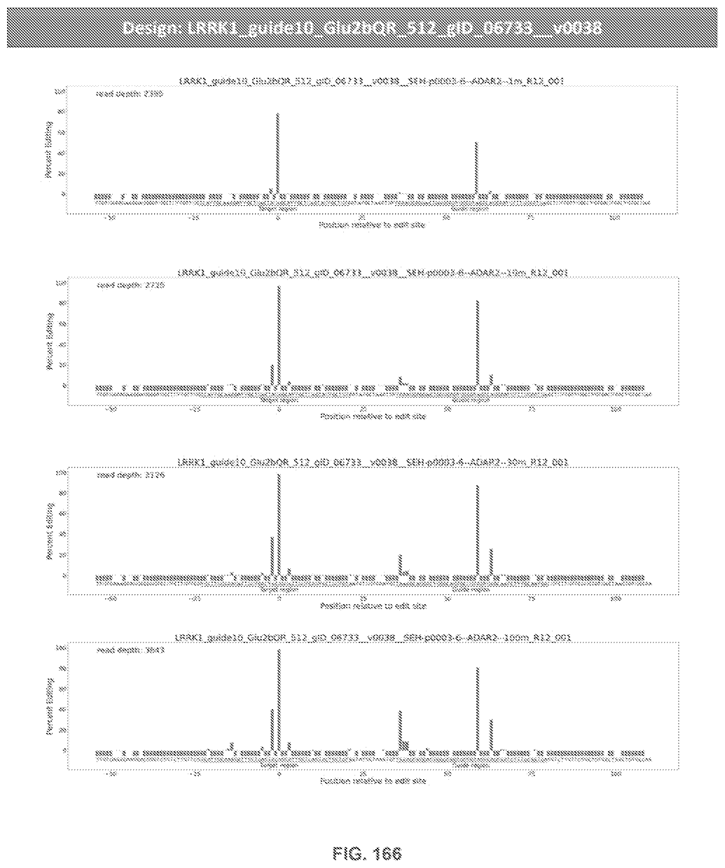

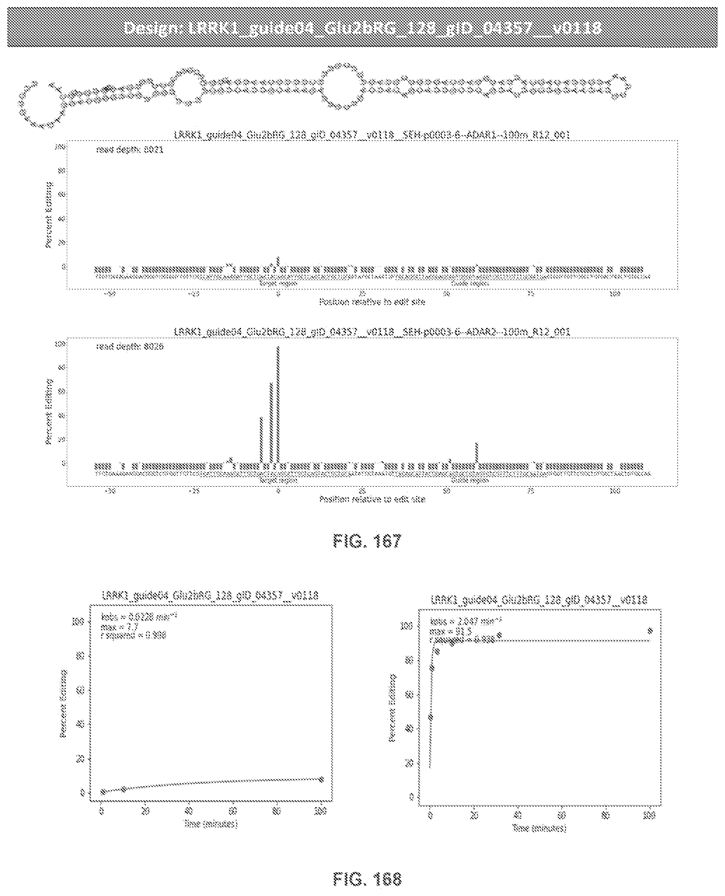

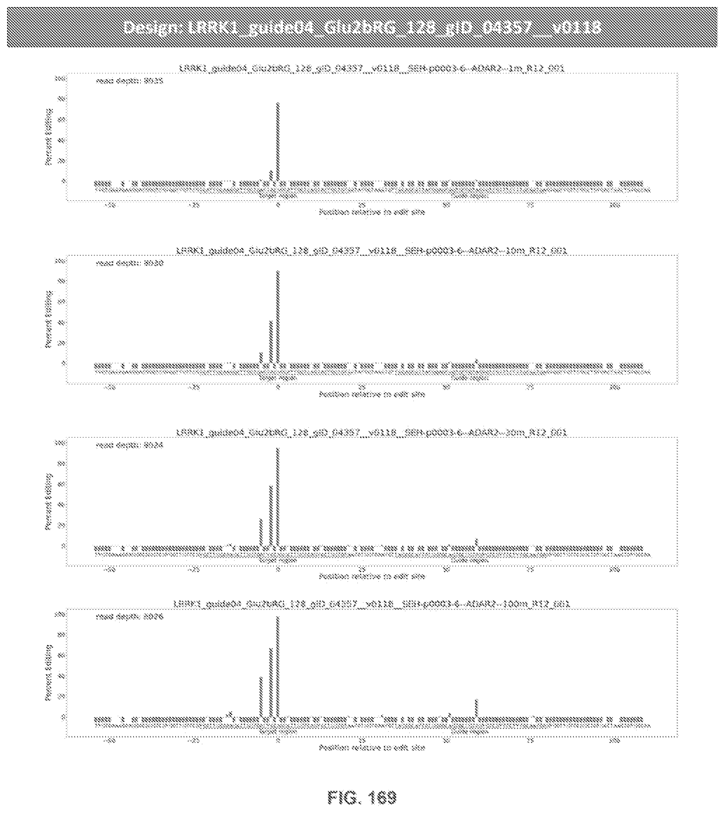

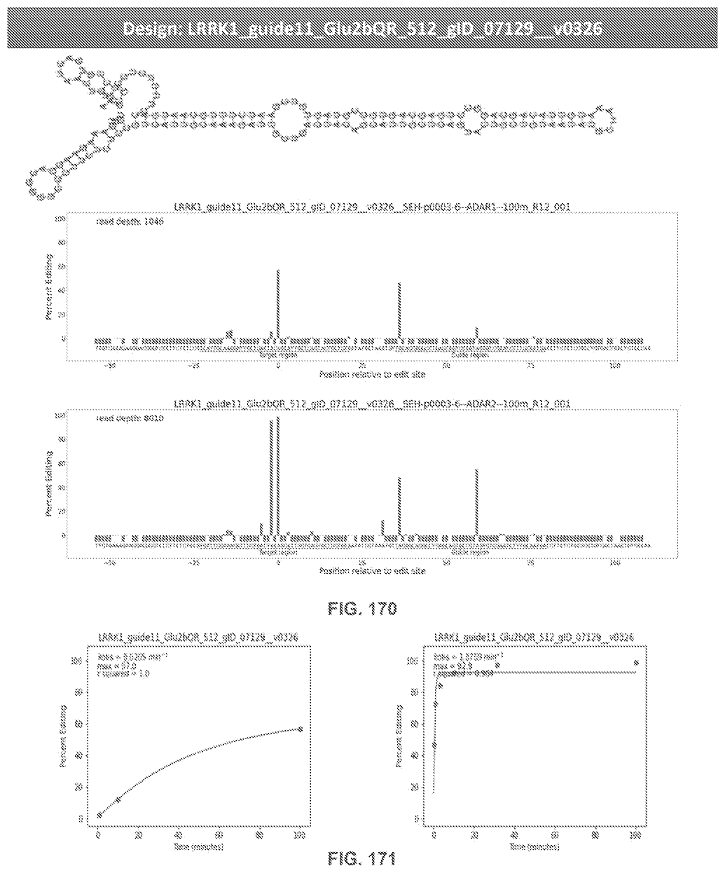

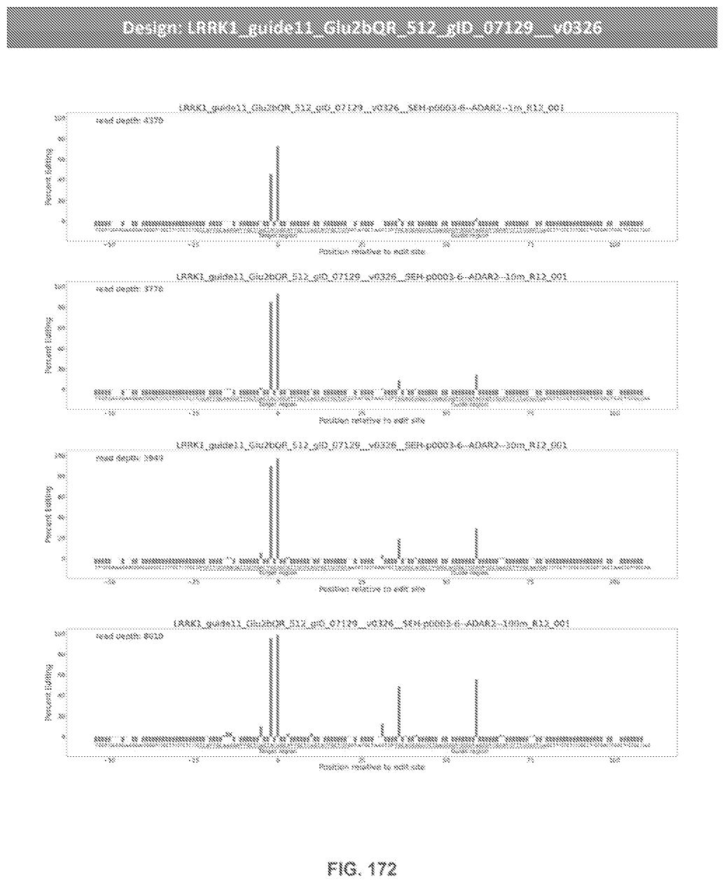

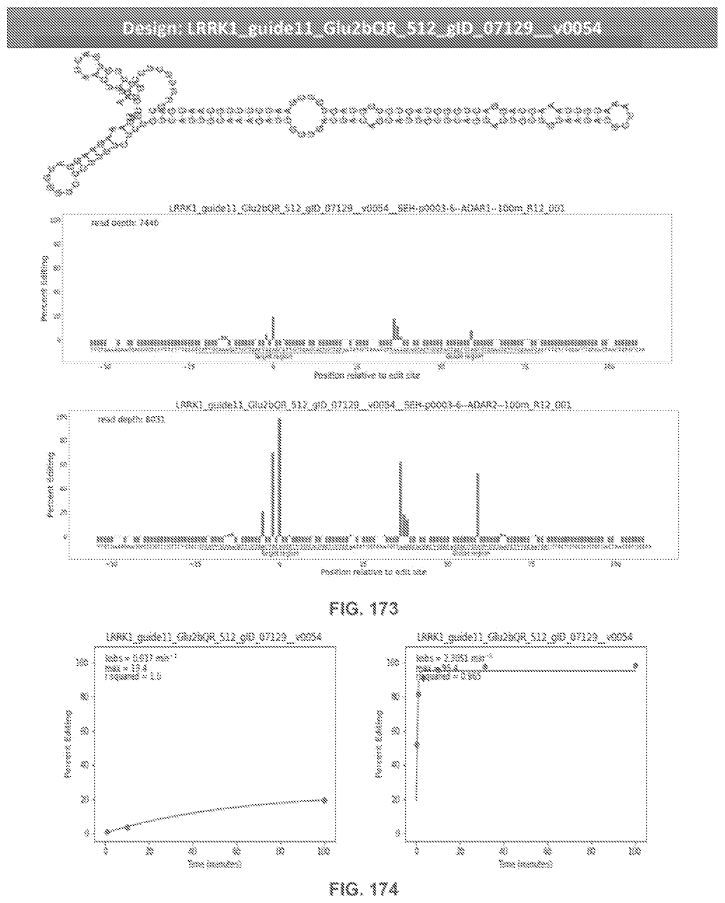

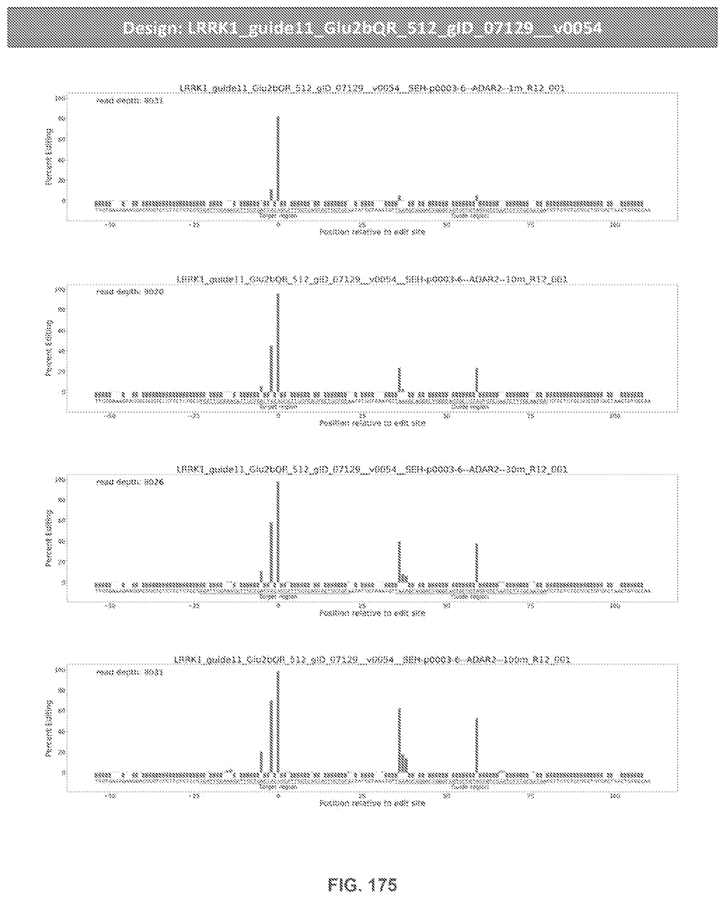

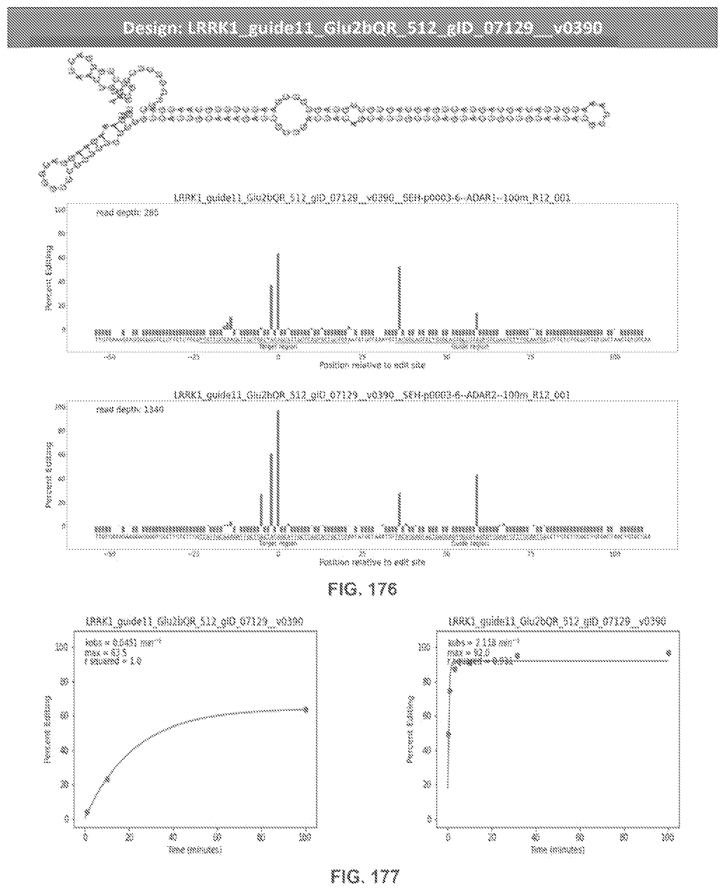

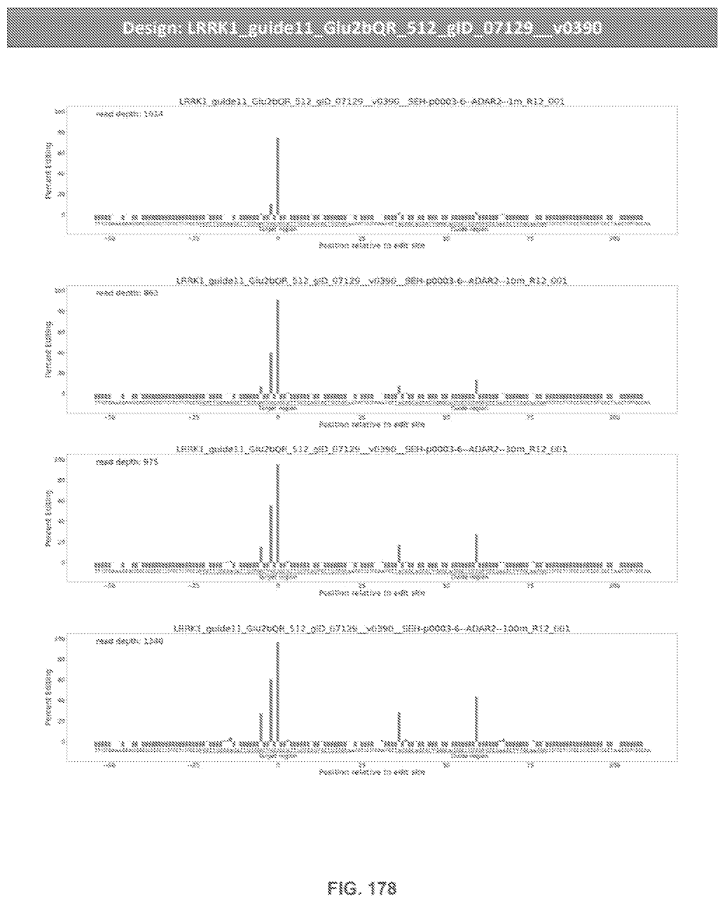

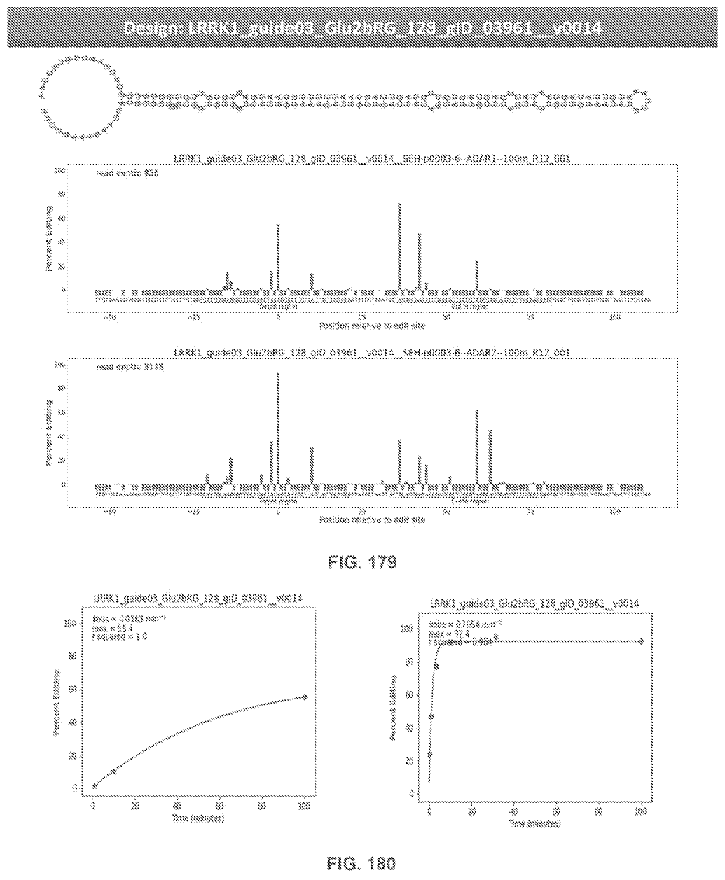

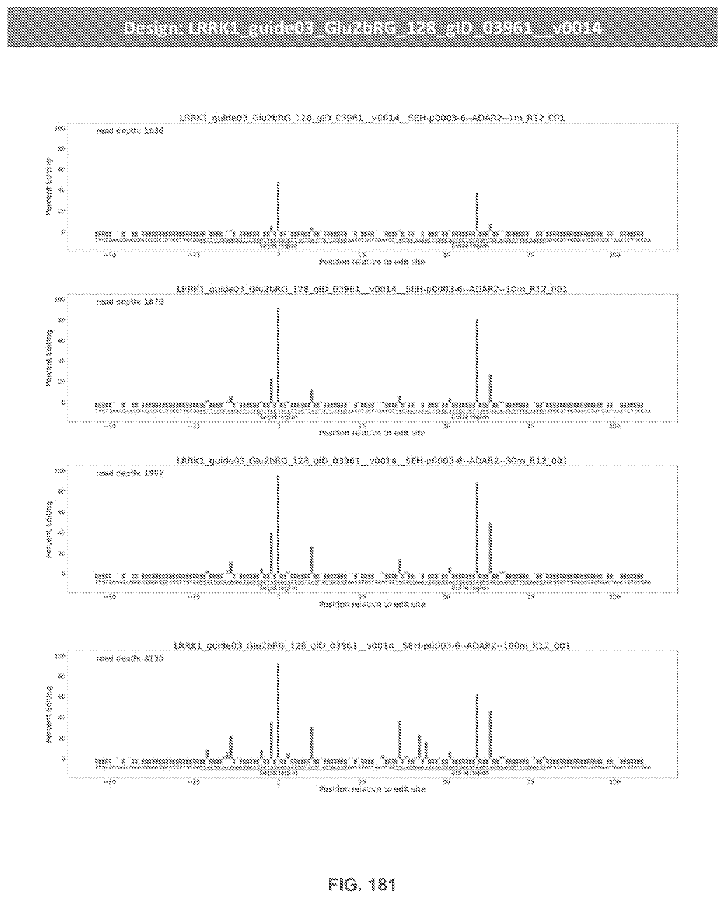

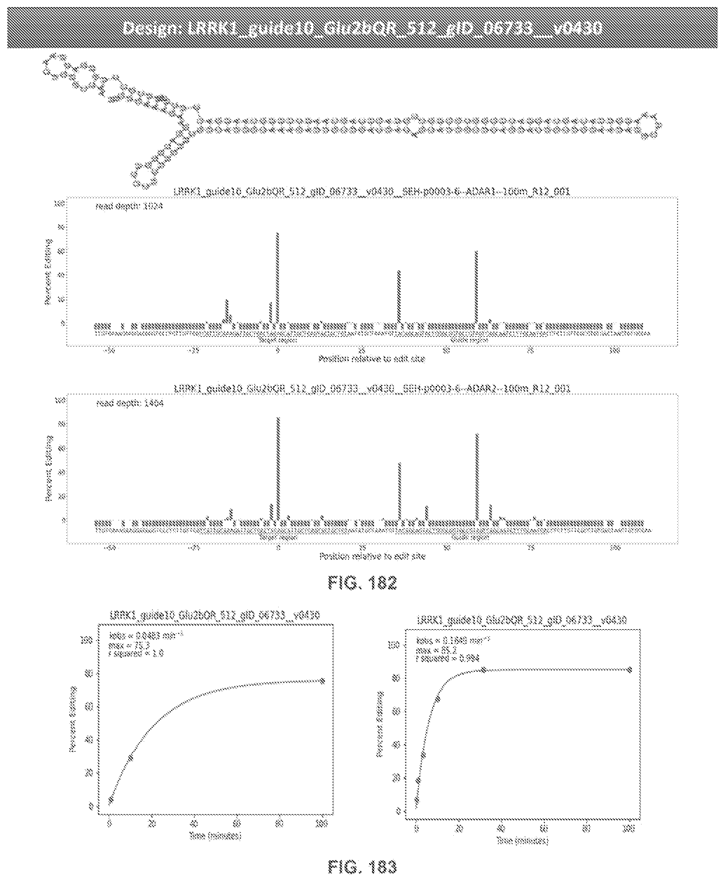

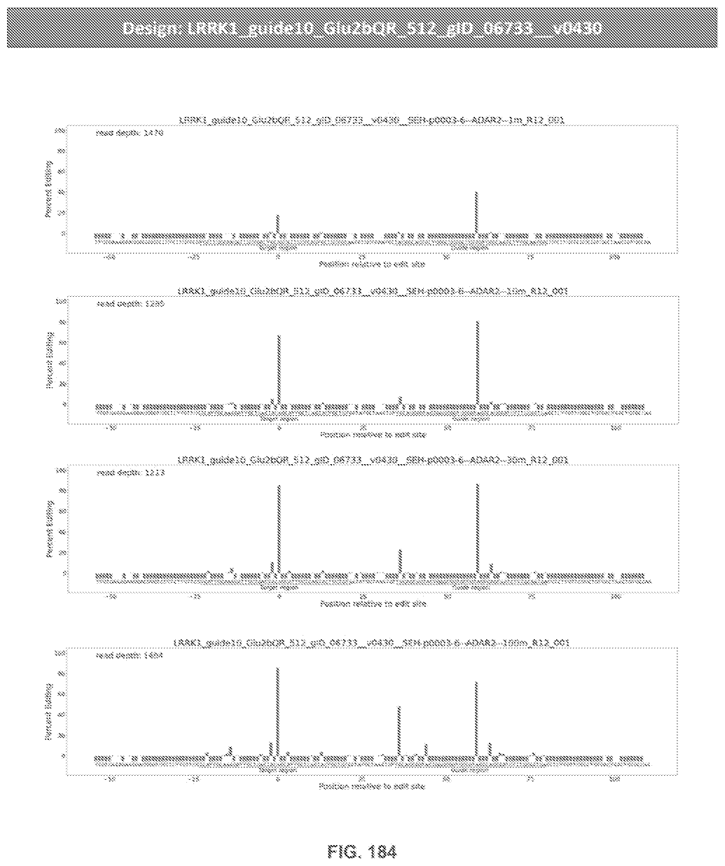

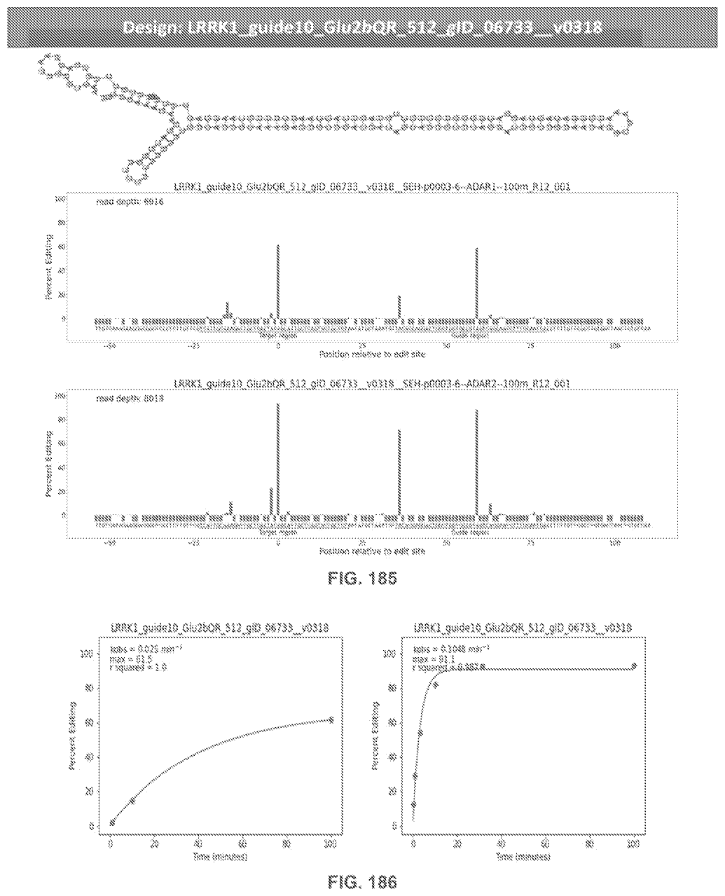

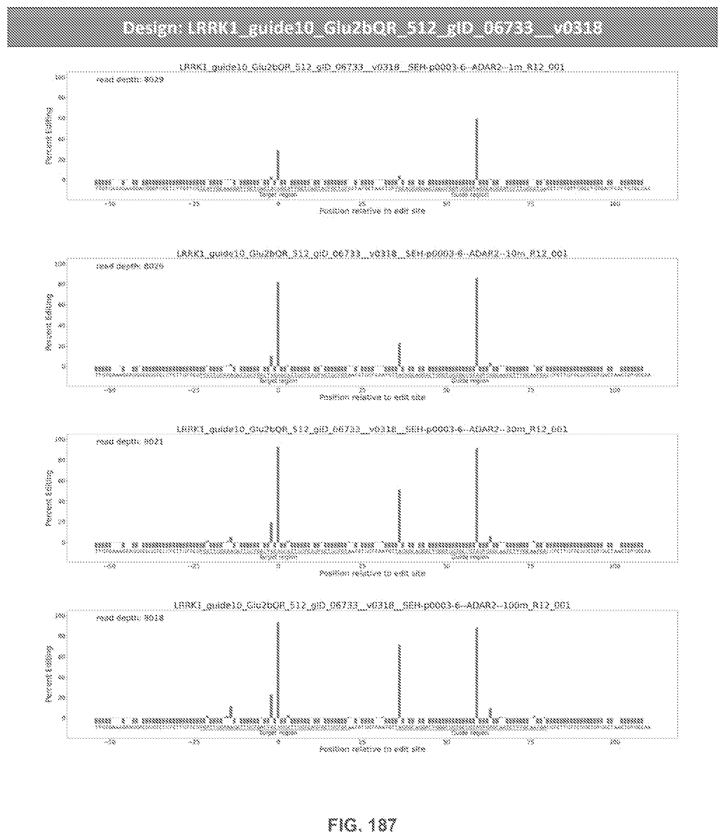

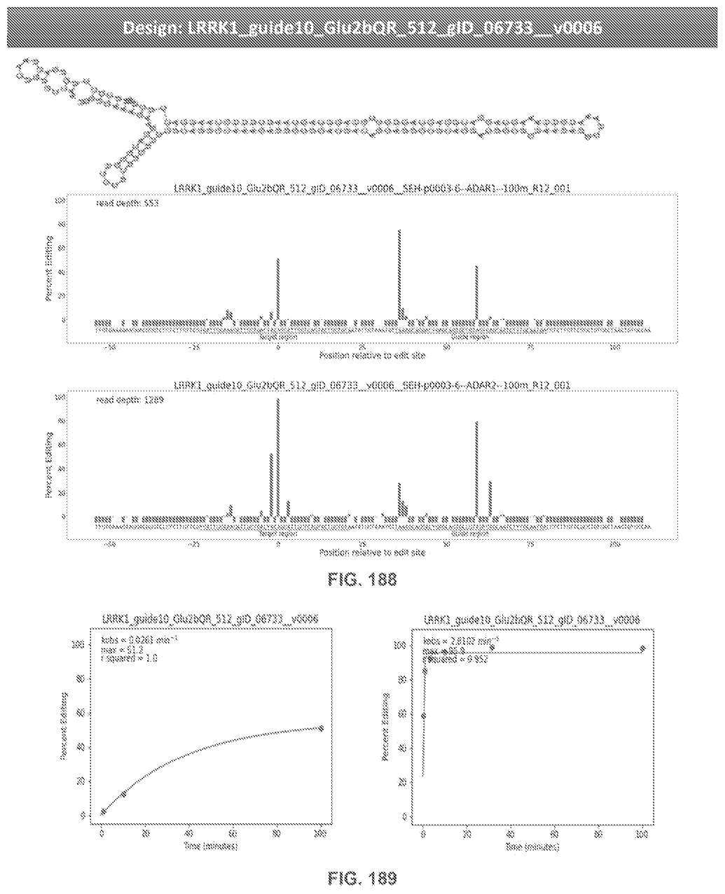

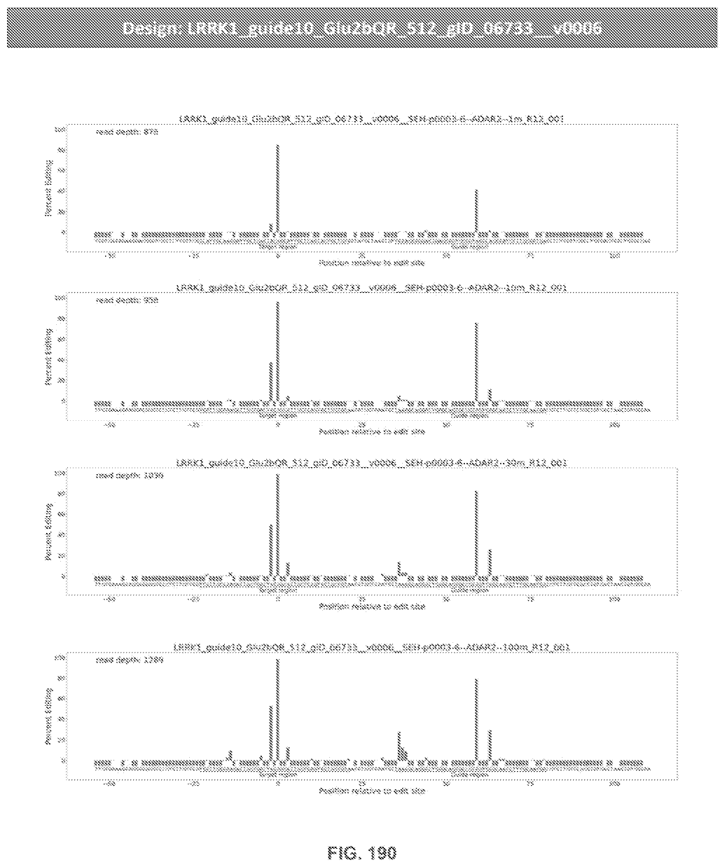

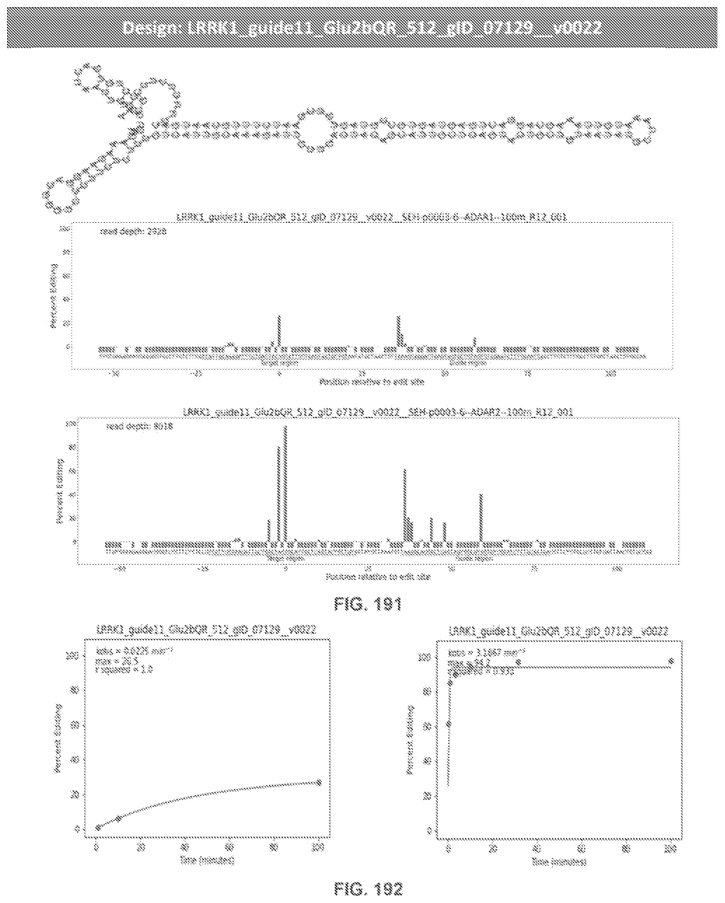

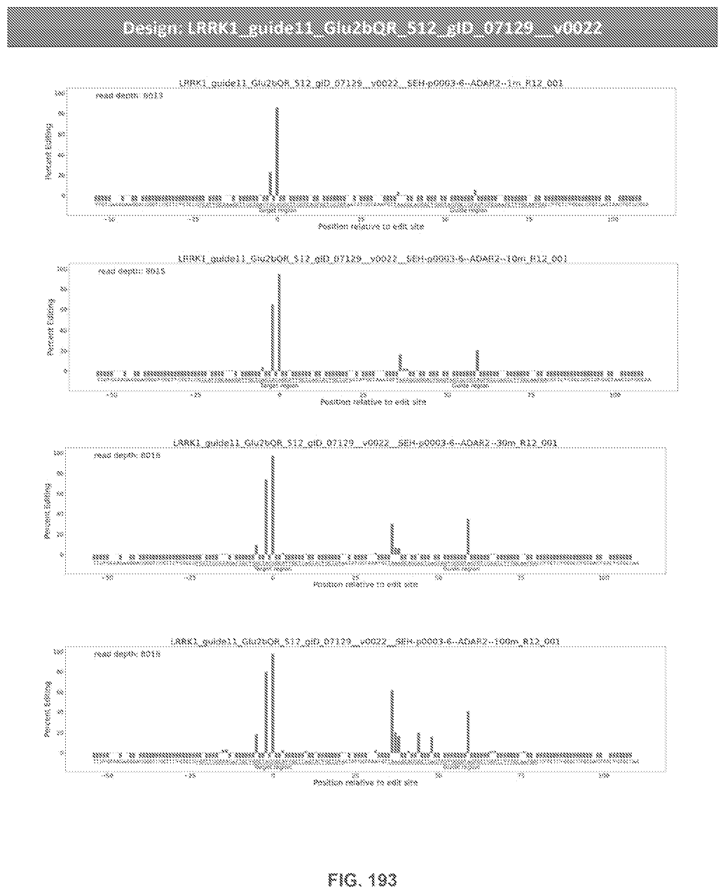

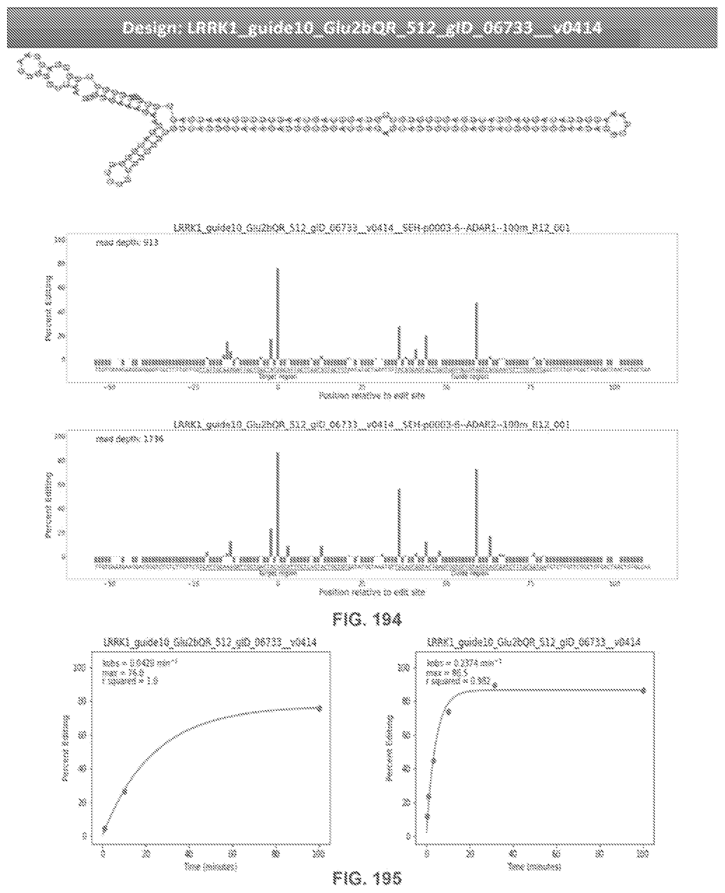

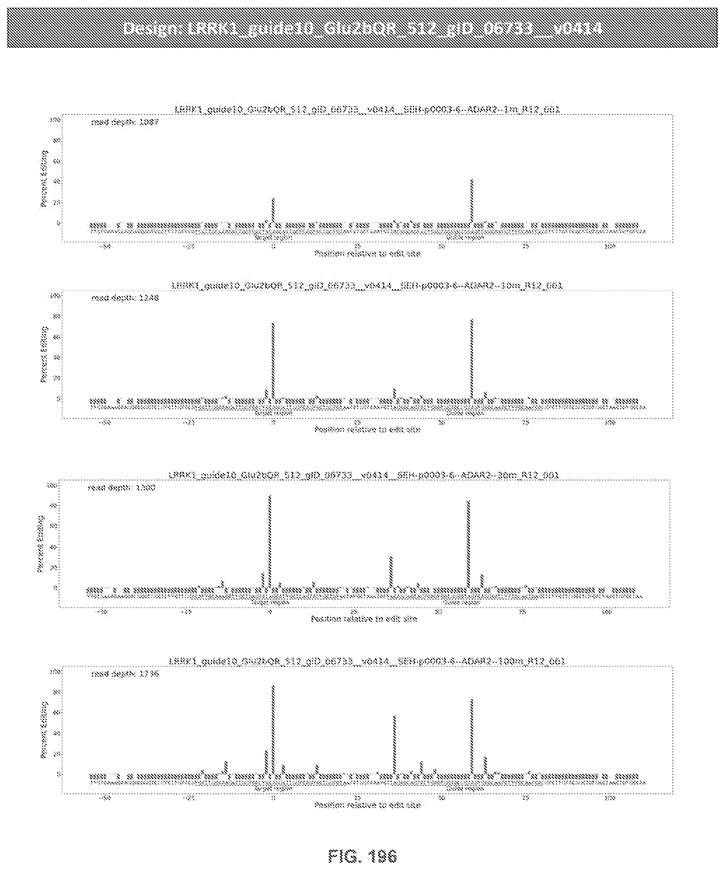

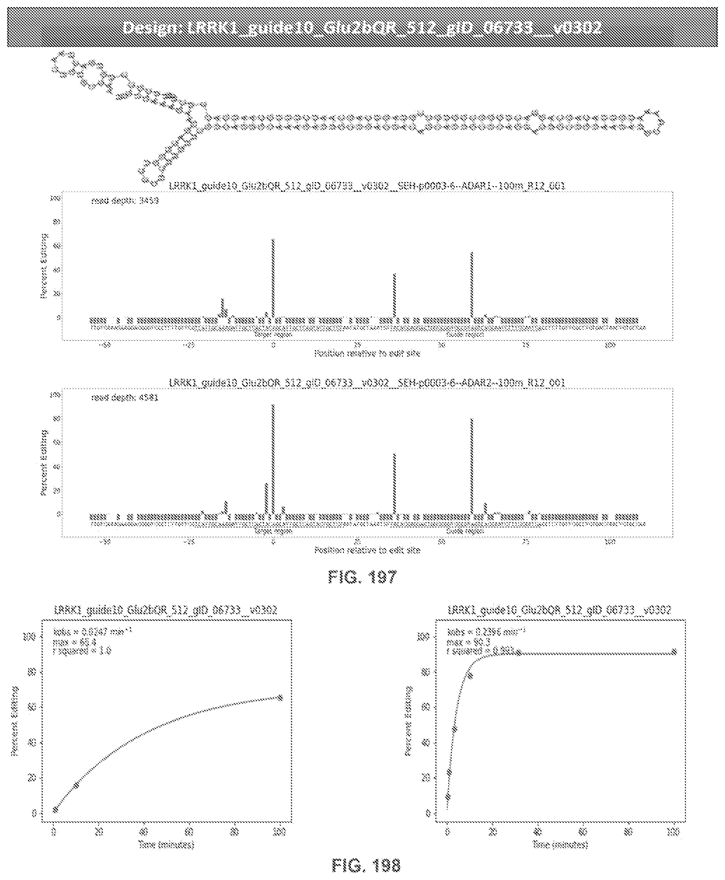

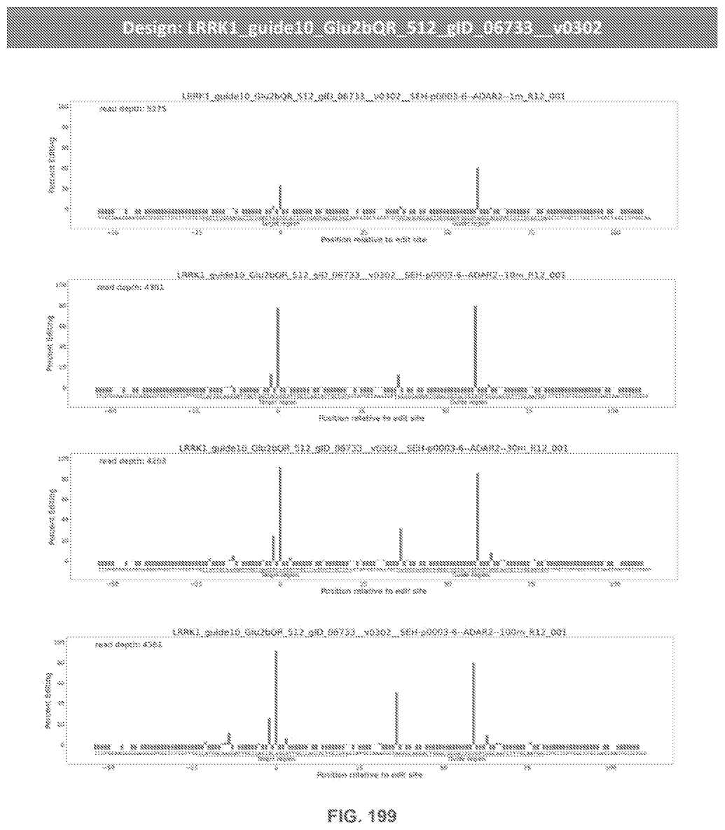

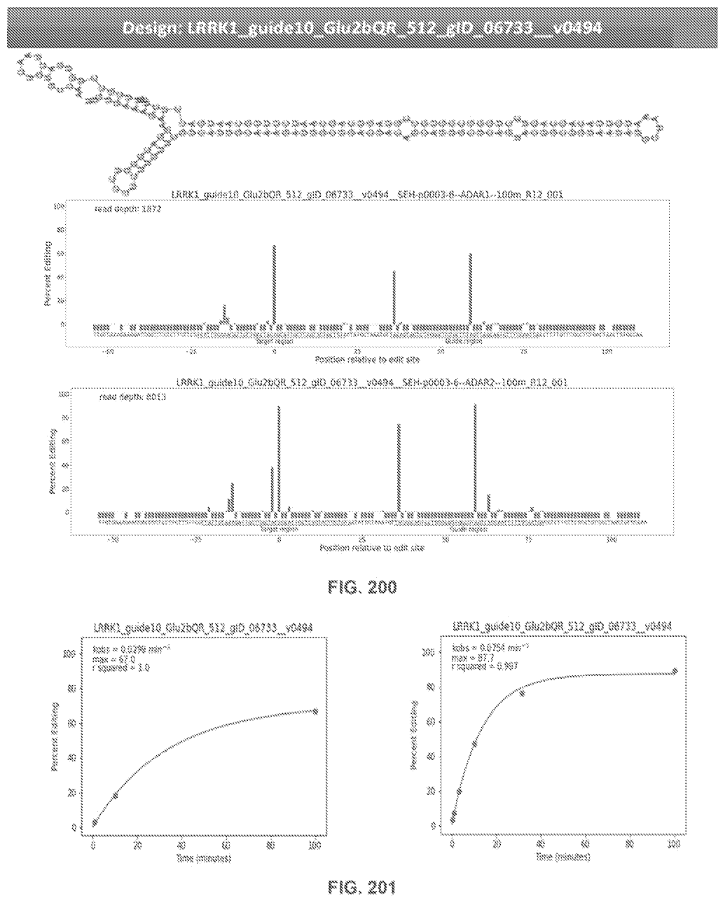

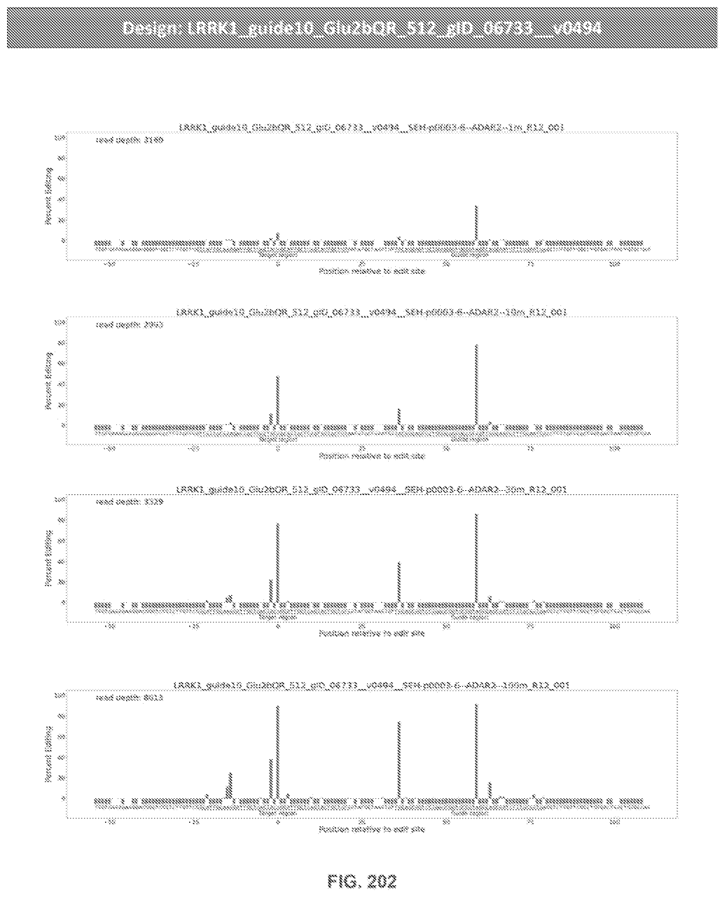

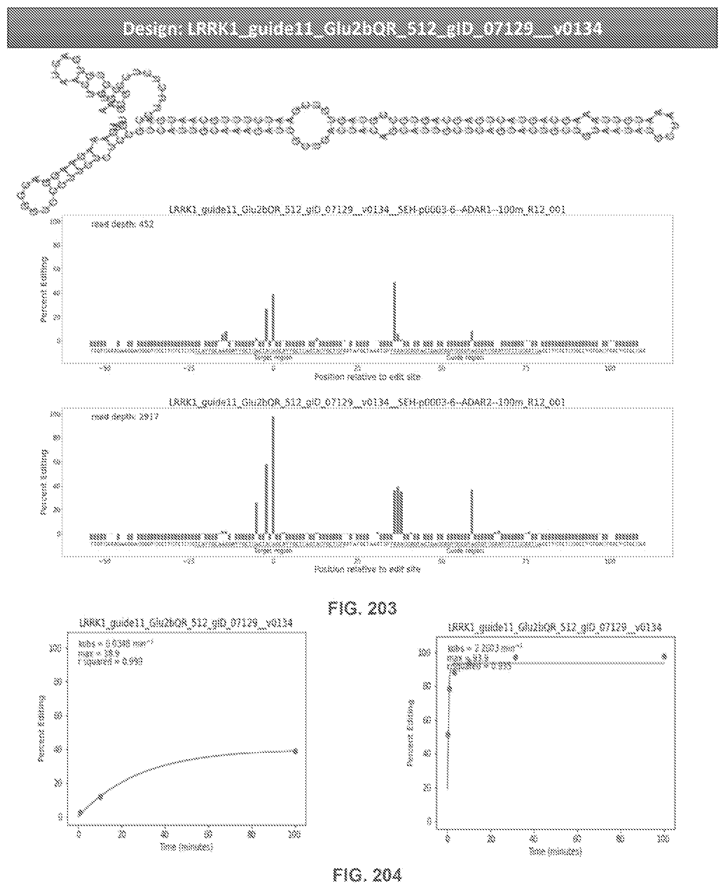

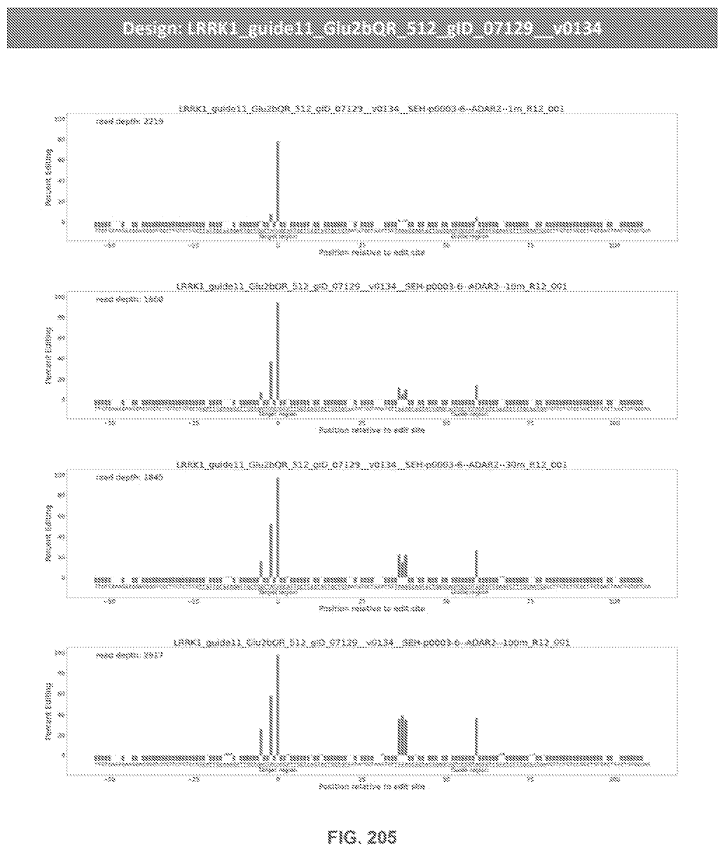

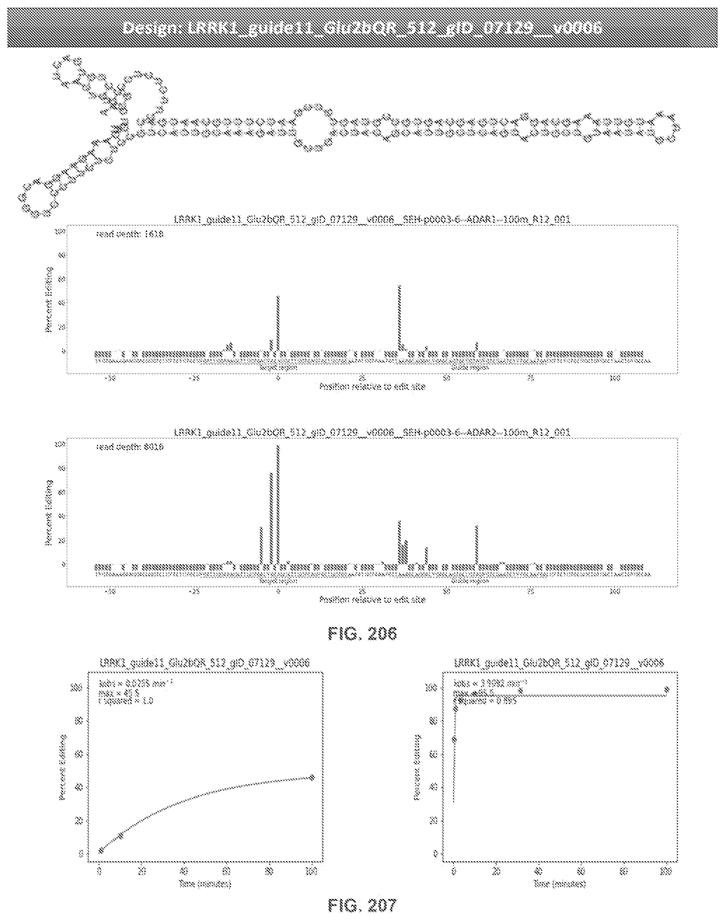

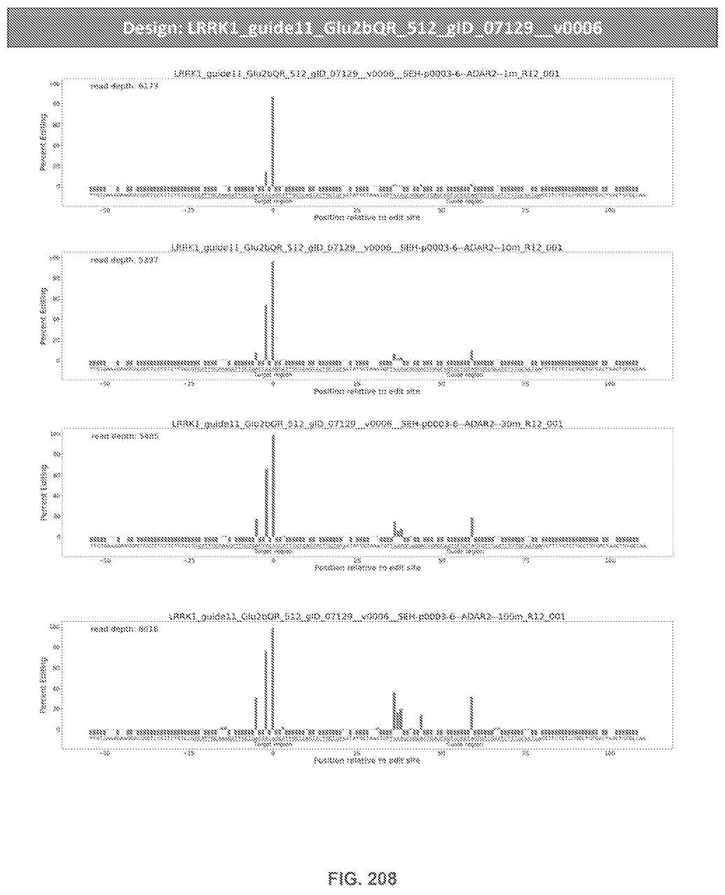

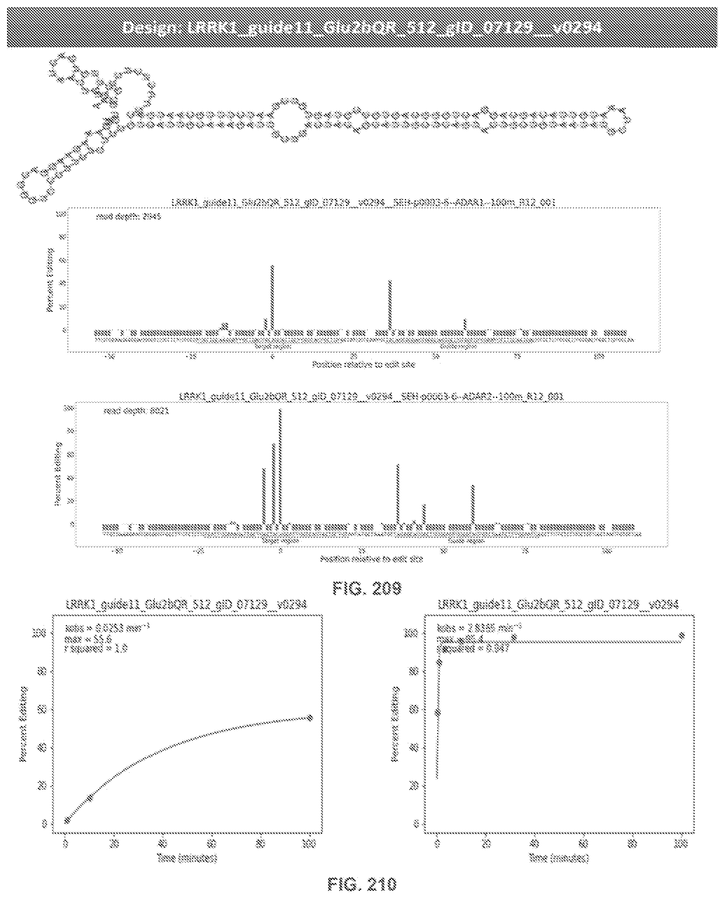

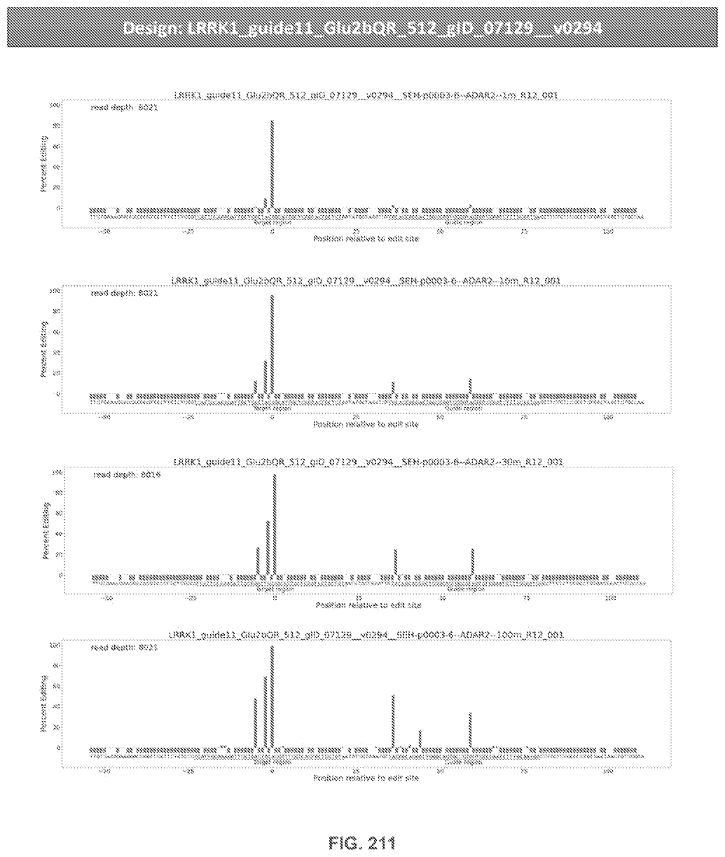

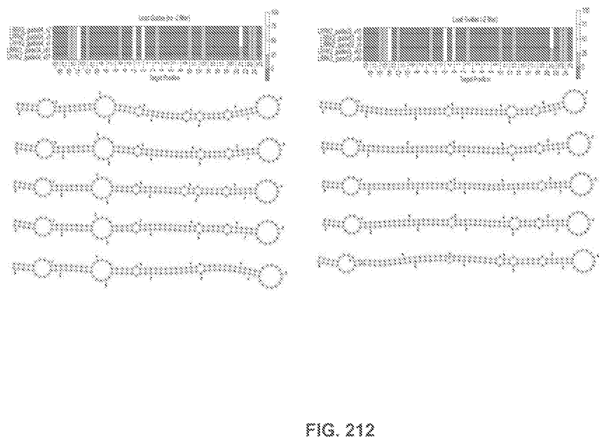

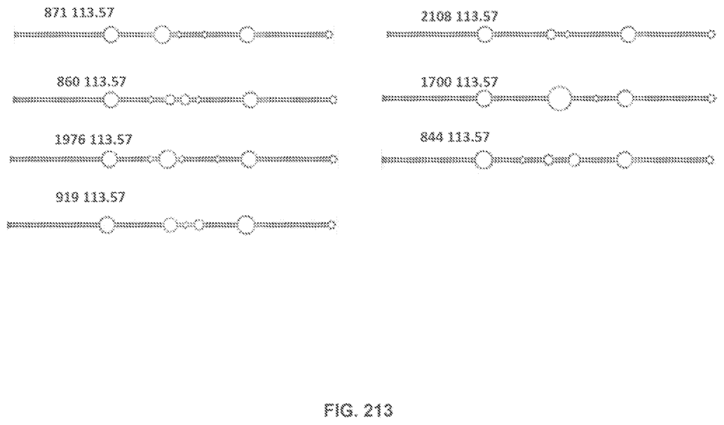

FIG. 110-FIG. 211 are structures of the self-annealing RNA structures that comprise the engineered guide RNAs of TABLE 17 and the target LRRK2 RNA. In FIG. 110-FIG. 211, graphs on the left show kinetics of ADAR1-mediated RNA editing and graphs on the right show kinetics of ADAR2-mediated RNA editing. Thus, these figures show the structural features formed upon hybridization of an engineered guide RNA of the present disclosure to target LRRK2 RNA. The target A was positioned towards the center of the guide-target RNA scaffold. These figures also show ADAR1 and ADAR2 on-target and off-target editing at 100 min ADAR1 and ADAR2 kinetics, and a timecourse of ADAR2 on-target and off-target editing at 1 min, 10 min, 30 min, and 100 min. These plots show the percentage editing as a function of time for each engineered polynucleotide as determined by sequencing, and the editing at the target A to be edited (“0” on the x-axis) and at RNA editing at off-target positions (represented as black bars at positions that are not “0”).

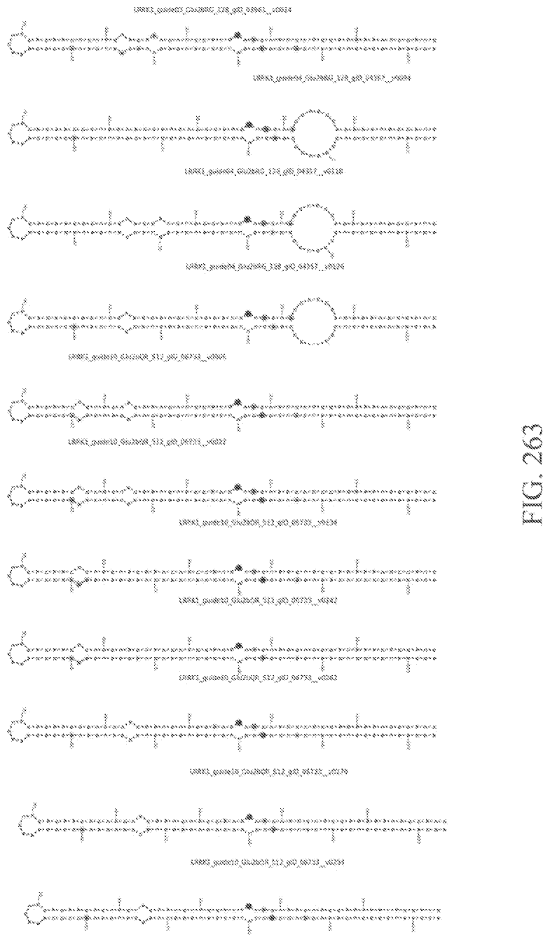

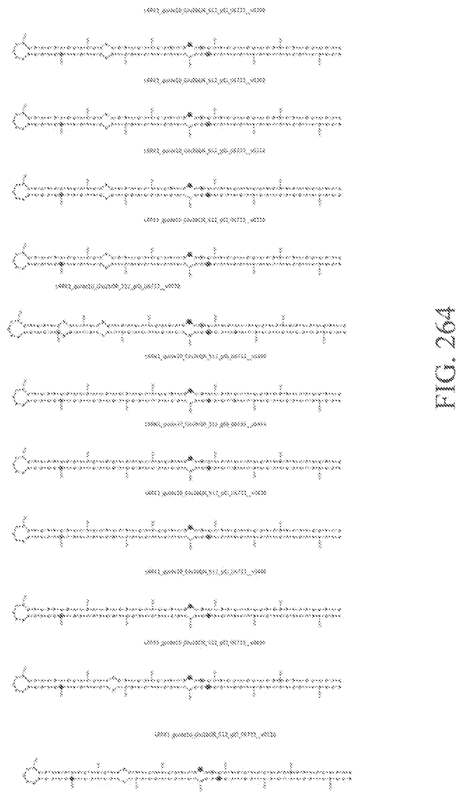

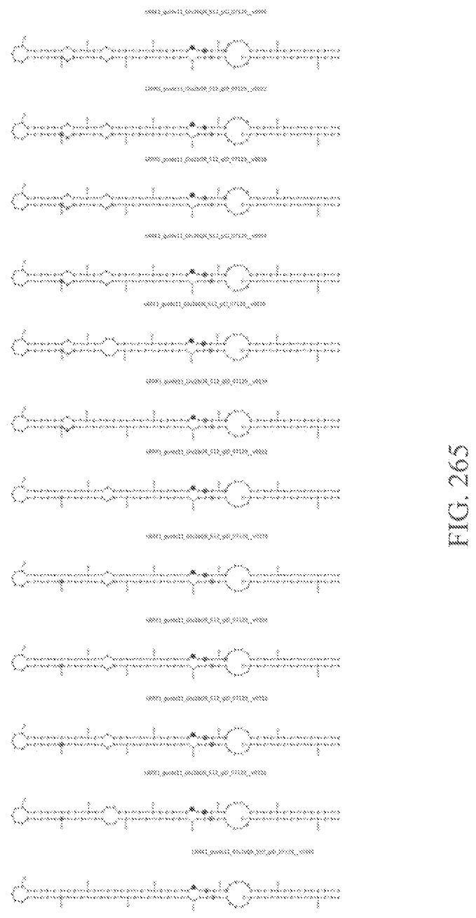

Exemplary guide RNA sequences corresponding to FIG. 110-FIG. 211 are shown in TABLE 17. FIG. 263-FIG. 265 shows diagrams of the guide-target RNA scaffold, highlighting the various structural features provided for in the engineered guide RNAs of TABLE 17. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 17
Exemplary Engineered Guide RNAs Targeting LRRK2 mRNA, 45 mers
for In Cis-Editing Experiments

| SEQ | | | | |
|---|---|---|---|---|
| ID | | | | |
| NO | ID | Sequence | Structural Features (target/guide) | Metrics |
| SEQ | LRRK1_ | TACAGCAGTACT | 1/1 A/C mismatch at 0 position | ADAR1 on target: 73.70% |
| ID | guide10_ | GGGTGGTGCCGT | 1/1 U/G wobble base pair at +4 position | ADAR2 on target: 86.88% |
| NO: | Glu2bRG_ | AGTCAGCAATCT | 1/1 U/G wobble base pair at +5 position | ADAR1 specificity: 1.2 |
| 111 | 512_ | TTGCAATGA | 1/1 G/U wobble base pair at +6 position | ADAR2 specificity: 1.41 |
| | gID_ | | 1/1 U/G wobble base pair at +8 position | |
| | 06733_ | | | |
| | v0446 | | | |
| SEQ | LRRK1_ | TACAGCAGGACT | 4/4 symmetric bulge at −6 position | ADAR1 on target: 65.40% |
| ID | guide11_ | GAGCAGTGCCGT | (GCUG-GUCG) | ADAR2 on target: 99.13% |
| NO: | Glu2bRG_ | AGTGTCGAATCT | 1/1 A/C mismatch at 0 position | ADAR1 specificity: 1.28 |
| 112 | 512_ | TTGCAATGA | 1/1 U/G wobble base pair at +4 position | ADAR2 specificity: 0.89 |
| | gID_ | | 1/1 A/G mismatch at +13 position | |
| | 07219_ | | | |
| | v0262 | | | |
| SEQ | LRRK1_ | TAAAGCAGGACT | 1/1 A/C mismatch at 0 position | ADAR1 on target: 33.17% |
| ID | guide10_ | GAGTAGTGCCGT | 1/1 U/G wobble base pair at +4 position | ADAR2 on target: 98.42% |
| NO: | Glu2bQR_ | AGTCAGCAATCT | 1/1 G/U wobble base pair at +6 position | ADAR1 specificity: 1.05 |
| 113 | 512_ | TTGCAATGA | 1/1 A/G mismatch at +13 position | ADAR2 specificity: 0.99 |
| | gID_ | | | |
| | 06733_ | | | |
| | v0022 | | | |
| SEQ | LRRK1_ | TACAGCAGTACT | 6/6 symmetric internal loop at −6 | ADAR1 on target: 62.41% |
| ID | guide04_ | GAGCAGTGCCGT | position (UUGCUG-GUCGUU) | ADAR2 on target: 96.74% |
| NO: | Glu2bRG_ | AGTGTCGTTTCT | 1/1 A/C mismatch at 0 position | ADAR1 specificity: 1.07 |
| 114 | 128_ | TTGCAATGA | 1/1 U/G wobble base pair at +4 position | ADAR2 specificity: 0.91 |
| | gID_ | | | |
| | 04357_ | | | |
| | v0094 | | | |
| SEQ | LRRK1_ | TACAGCATTACT | 6/6 symmetric internal loop at −6 | ADAR1 on target: 12.45% |
| ID | guide04_ | GAGCAGTGCCGT | position (UUGCUG-GUCGUU) | ADAR2 on target: 98.98% |
| NO: | Glu2bRG_ | AGTGTCGTTTCT | 1/1 A/C mismatch at 0 position | ADAR1 specificity: 0.89 |
| 115 | 128_ | TTGCAATGA | 1/1 U/G wobble base pair at +4 position | ADAR2 specificity: 0.9 |
| | gID_ | | 1/1 C/U mismatch at +14 position | |
| | 04357_ | | | |
| | v0126 | | | |
| SEQ | LRRK1_ | TACAGCAGGACT | 4/4 symmetric bulge at −6 position | ADAR1 on target: 46.44% |
| ID | guide11_ | GAGTAGTGCCGT | (GCUG-GUCG) | ADAR2 on target: 98.62% |
| NO: | Glu2bQR_ | AGTGTCGAATCT | 1/1 A/C mismatch at 0 position | ADAR1 specificity: 1.22 |
| 116 | 512_ | TTGCAATGA | 1/1 U/G wobble base pair at +4 position | ADAR2 specificity: 0.9 |
| | gID_ | | 1/1 G/U wobble base pair at +6 position | |
| | 07129_ | | 1/1 A/G mismatch at +13 position | |
| | v0278 | | | |
| SEQ | LRRK1_ | TACAGCAGGACT | 1/1 A/C mismatch at 0 position | ADAR1 on target: 72.36% |
| ID | guide10_ | GAGCGGTGCCGT | 1/1 U/G wobble base pair at +4 position | ADAR2 on target: 85.91% |
| NO: | Glu2bQR_ | AGTCAGCAATCT | 1/1 U/G wobble base pair at +5 position | ADAR1 specificity: 1.29 |
| 117 | 512_ | TTGCAATGA | 1/1 A/G mismatch at +13 position | ADAR2 specificity: 1.16 |
| | gID_ | | | |
| | 06733_ | | | |
| | v0270 | | | |
| SEQ | LRRK1_ | TACAGCAGTACT | 1/1 A/C mismatch at 0 position | ADAR1 on target: 69.70% |
| ID | guide10_ | GAGCGGTGCCGT | 1/1 U/G wobble base pair at +4 position | ADAR2 on target: 83.27% |
| NO: | Glu2bQR_ | AGTCAGCAATCT | 1/1 U/G wobble base pair at +5 position | ADAR1 specificity: 1.18 |
| 118 | 512_ | TTGCAATGA | | ADAR2 specificity: 1.16 |
| | gID_ | | | |
| | 06733_ | | | |
| | v0398 | | | |
| SEQ | LRRK1_ | TACAGCAGGACT | 1/1 A/C mismatch at 0 position | ADAR1 on target: 74.90% |
| ID | guide10_ | GGGTGATGCCGT | 1/1 U/G wobble base pair at +5 position | ADAR2 on target: 83.31% |
| NO: | Glu2bQR_ | AGTCAGCAATCT | 1/1 G/U wobble base pair at +6 position | ADAR1 specificity: 1.25 |
| 119 | 512_ | TTGCAATGA | 1/1 U/G wobble base pair at +8 position | ADAR2 specificity: 1.15 |
| | gID_ | | 1/1 A/G mismatch at +13 position | |
| | 06733_ | | | |
| | v0314 | | | |
| SEQ | LRRK1_ | TAAAGCAGTACT | 1/1 A/C mismatch at 0 position | ADAR1 on target: 56.32% |
| ID | guide10_ | GAGCGGTGCCGT | 1/1 U/G wobble base pair at +4 position | ADAR2 on target: 92.45% |
| NO: | Glu2bQR_ | AGTCAGCAATCT | 1/1 U/G wobble base pair at +5 position | ADAR1 specificity: 1.13 |
| 120 | 512_ | TTGCAATGA | | ADAR2 specificity: 1.24 |
| | gID_ | | | |
| | 06733_ | | | |
| | v0142 | | | |
| SEQ | LRRK1_ | TACAGCAGTCCT | 1/1 A/C mismatch at 0 position | ADAR1 on target: 62.79% |
| ID | guide10_ | GGGTGGTGCCGT | 1/1 U/G wobble base pair at +4 position | ADAR2 on target: 89.69% |
| NO: | Glu2bQR_ | AGTCAGCAATCT | 1/1 U/G wobble base pair at +5 position | ADAR1 specificity: 1.26 |
| 121 | 512_ | TTGCAATGA | 1/1 G/U wobble base pair at +6 position | ADAR2 specificity: 1.12 |
| | gID_ | | 1/1 U/G wobble base pair at +8 position | |
| | 06733_ | | 1/1 U/C mismatch at +12 position | |
| | v0510 | | | |
| SEQ | LRRK1_ | TACAGCAGGACT | 4/4 symmetric bulge at −6 position | ADAR1 on target: 36.58% |
| ID | guide11_ | GGGTAGTGCCGT | (GCUG-GUCG) | ADAR2 on target: 98.87% |
| NO: | Glu2bQR_ | AGTGTCGAATCT | 1/1 A/C mismatch at 0 position | ADAR1 specificity: 1.19 |
| 122 | 512_ | TTGCAATGA | 1/1 U/G wobble base pair at +4 position | ADAR2 specificity: 0.92 |
| | gID_ | | 1/1 G/U wobble base pair at +6 position | |
| | 07129_ | | 1/1 U/G wobble base pair at +8 position | |
| | v0310 | | 1/1 A/G mismatch at +13 position | |
| SEQ | LRRK1_ | TACAGCAGGACT | 1/1 A/C mismatch at 0 position | ADAR1 on target: 73.18% |
| ID | guide10_ | GAGCAGTGCCGT | 1/1 U/G wobble base pair at +4 position | ADAR2 on target: 97.93% |
| NO: | Glu2bQR_ | AGTCAGCAATCT | 1/1 A/G mismatch at +13 position | ADAR1 specificity: 1.29 |
| 123 | 512_ | TTGCAATGA | | ADAR2 specificity: 1.08 |
| | gID_ | | | |
| | 06733_ | | | |
| | v0262 | | | |
| SEQ | LRRK1_ | TAAAGCAGTACT | 1/1 A/C mismatch at 0 position | ADAR1 on target: 43.41% |
| ID | guide10_ | GAGCAGTGCCGT | 1/1 U/G wobble base pair at +4 position | ADAR2 on target: 97.78% |
| NO: | Glu2bQR_ | AGTCAGCAATCT | | ADAR1 specificity: 0.96 |
| 124 | 512_ | TTGCAATGA | | ADAR2 specificity: 1.21 |
| | gID_ | | | |
| | 06733_ | | | |
| | v0134 | | | |
| SEQ | LRRK1_ | TAAAGCAGGCCT | 4/4 symmetric bulge at −6 position | ADAR1 on target: 33.33% |
| ID | guide11_ | GAGCAGTGCCGT | (GCUG-GUCG) | ADAR2 on target: 98.79% |
| NO: | Glu2bQR_ | AGTGTCGAATCT | 1/1 A/C mismatch at 0 position | ADAR1 specificity: 1.15 |
| 125 | 512_ | TTGCAATGA | 1/1 U/G wobble base pair at +4 position | ADAR2 specificity: 0.91 |
| | gID_ | | 2/2 symmetric bulge at +12 position | |
| | 07129_ | | (UA-GC) | |
| | v0070 | | | |
| SEQ | LRRK1_ | TAAAGCAGGACT | 4/4 symmetric bulge at −6 position | ADAR1 on target: 31.97% |
| ID | guide11_ | GGGCAGTGCCGT | (GCUG-GUCG) | ADAR2 on target: 99.09% |
| NO: | Glu2bQR_ | AGTGTCGAATCT | 1/1 A/C mismatch at 0 position | ADAR1 specificity: 1.13 |
| 126 | 512_ | TTGCAATGA | 1/1 U/G wobble base pair at +4 position | ADAR2 specificity: 0.97 |
| | gID_ | | 1/1 U/G wobble base pair at +8 position | |
| | 07129_ | | 1/1 A/G mismatch at +13 position | |
| | v0038 | | | |
| SEQ | LRRK1_ | TACAGCAGGACT | 1/1 A/C mismatch at 0 position | ADAR1 on target: 74.08% |
| ID | guide10_ | GGGCGATGCCGT | 1/1 U/G wobble base pair at +5 position | ADAR2 on target: 83.71% |
| NO: | Glu2bQR_ | AGTCAGCAATCT | 1/1 U/G wobble base pair at +8 position | ADAR1 specificity: 1.18 |
| 127 | 512_ | TTGCAATGA | 1 A/G mismatch at +13 position | ADAR2 specificity: 1.08 |
| | gID_ | | | |
| | 06733_ | | | |
| | v0298 | | | |
| SEQ | LRRK1__ | TACAGCAGGACT | 1/1 A/C mismatch at 0 position | ADAR1 on target: 63.43% |
| ID | guide10_ | GGGCAGTGCCGT | 1/1 U/G wobble base pair at +4 position | ADAR2 on target: 96.93% |
| NO: | Glu2bQR_ | AGTCAGCAATCT | 1/1 U/G wobble base pair at +8 position | ADAR1 specificity: 1.2 |
| 128 | 512_ | TTGCAATGA | 1/1 A/G mismatch at +13 position | ADAR2 specificity: 1.27 |
| | gID_ | | | |
| | 06733_ | | | |
| | v0294 | | | |
| SEQ | LRRK1_ | TAAAGCAGGACT | 1/1 A/C mismatch at 0 position | ADAR1 on target: 42.45% |
| ID | guide10_ | GGGCAGTGCCGT | 1/1 U/G wobble base pair at +4 position | ADAR2 on target: 98.70% |
| NO: | Glu2bQR_ | AGTCAGCAATCT | 1/1 U/G wobble base pair at +8 position | ADAR1 specificity: 1.1 |
| 129 | 512_ | TTGCAATGA | 1/1 A/G mismatch at +13 position | ADAR2 specificity: 1.2 |
| | gID_ | | | |
| | 06733_ | | | |
| | v0038 | | | |
| SEQ | LRRK1_ | TACAGCATTACG | 6/6 symmetric internal loop at −6 | ADAR1 on target: 7.65% |
| ID | guide04_ | GAGCAGTGCCGT | position (UUGCUG-GUCGUU) | ADAR2 on target: 97.53% |
| NO: | Glu2bRG_ | AGTGTCGTTTCT | 1/1 A/C mismatch at 0 position | ADAR1 specificity: 0.99 |
| 130 | 128_ | TTGCAATGA | 1/1 U/G wobble base pair at +4 position | ADAR2 specificity: 0.92 |
| | gID_ | | 1/1 A/G mismatch at +10 position | |
| | 04357_ | | 1/1 C/U mismatch at +14 position | |
| | v0118 | | | |
| SEQ | LRRK1_ | TACAGCAGGCCT | 4/4 symmetric bulge at −6 position | ADAR1 on target: 56.98% |
| ID | guide11_ | GAGCAGTGCCGT | (GCUG-GUCG) | ADAR2 on target: 98.63% |
| NO: | Glu2bQR_ | AGTGTCGAATCT | 1/1 A/C mismatch at 0 position | ADAR1 specificity: 1.28 |
| 131 | 512_ | TTGCAATGA | 1/1 U/G wobble base pair at +4 position | ADAR2 specificity: 0.9 |
| | gID_ | | 2/2 symmetric bulge at +12 position | |
| | 07129_ | | (UA-GC) | |
| | v0326 | | | |
| SEQ | LRRK1_ | TAAAGCAGGACT | 4/4 symmetric bulge at −6 position | ADAR1 on target: 19.41% |
| ID | guide11_ | GGGTAGTGCCGT | (GCUG-GUCG) | ADAR2 on target: 98.63% |
| NO: | Glu2bQR_ | AGTGTCGAATCT | 1/1 A/C mismatch at 0 position | ADAR1 specificity: 1.06 |
| 132 | 512_ | TTGCAATGA | 1/1 U/G wobble base pair at +4 position | ADAR2 specificity: 1 |
| | gID_ | | 1/1 G/U wobble base pair at +6 position | |
| | 07129_ | | 1/1 U/G wobble base pair at +8 position | |
| | v0054 | | 1/1 A/G mismatch at +13 position | |
| SEQ | LRRK1_ | TACAGCAGTACT | 4/4 symmetric bulge at −6 position | ADAR1 on target: 63.51% |
| ID | guide11_ | GAGCAGTGCCGT | (GCUG-GUCG) | ADAR2 on target: 96.87% |
| NO: | Glu2bQR_ | AGTGTCGAATCT | 1/1 A/C mismatch at 0 position | ADAR1 specificity: 1.01 |
| 133 | 512_ | TTGCAATGA | 1/1 U/G wobble base pair at +4 position | ADAR2 specificity: 1 |
| | gID_ | | | |
| | 07129_ | | | |
| | v0390 | | | |
| SEQ | LRRK1_ | TACAGCAATACC | 1/1 A/C mismatch at 0 position | ADAR1 on target: 55.37% |
| ID | guide03_ | GAGCAGTGCCGT | 1/1 U/G wobble base pair at +4 position | ADAR2 on target: 92.89% |
| NO: | Glu2bRG_ | AGTCAGCAATCT | 1/1 A/G mismatch at +10 position | ADAR1 specificity: 0.98 |
| 134 | 128_ | TTGCAATGA | 1/1 C/A mismatch at +14 position | ADAR2 specificity: 0.88 |
| | gID_ | | | |
| | 03961_ | | | |
| | v0014 | | | |
| SEQ | LRRK1_ | TACAGCAGTACT | 1/1 A/C mismatch at 0 position | ADAR1 on target: 75.29% |
| ID | guide10_ | GGGCGGTGCCGT | 1/1 U/G wobble base pair at +4 position | ADAR2 on target: 85.54% |
| NO: | Glu2bQR_ | AGTCAGCAATCT | 1/1 U/G wobble base pair at +5 position | ADAR1 specificity: 1.16 |
| 135 | 512_ | TTGCAATGA | 1/1 U/G wobble base pair at +8 position | ADAR2 specificity: 1.34 |
| | gID_ | | | |
| | 06733_ | | | |
| | v0430 | | | |
| SEQ | LRRK1_ | TACAGCAGGACT | 1/1 A/C mismatch at 0 position | ADAR1 on target: 61.52% |
| ID | guide10_ | GGGTGGTGCCGT | 1/1 U/G wobble base pair at +4 position | ADAR2 on target: 93.45% |
| NO: | Glu2bQR_ | AGTCAGCAATCT | 1/1 U/G wobble base pair at +5 position | ADAR1 specificity: 1.27 |
| 136 | 512_ | TTGCAATGA | 1/1 G/U wobble base pair at +6 position | ADAR2 specificity: 1.34 |
| | gID_ | | 1/1 U/G wobble base pair at +8 position | |
| | 06733_ | | 1/1 A/G mismatch at +13 position | |
| | v0318 | | | |
| SEQ | LRRK1_ | TAAAGCAGGACT | 1/1 A/C mismatch at 0 position | ADAR1 on target: 51.18% |
| ID | guide10_ | GAGCAGTGCCGT | 1/1 U/G wobble base pair at +4 position | ADAR2 on target: 98.68% |
| NO: | Glu2bQR_ | AGTCAGCAATCT | 1/1 A/G mismatch at +13 position | ADAR1 specificity: 1.2 |
| 137 | 512_ | TTGCAATGA | | ADAR2 specificity: 1.07 |
| | gID_ | | | |
| | 06733_ | | | |
| | v0006 | | | |
| SEQ | LRRK1_ | TAAAGCAGGACT | 4/4 symmetric bulge at −6 position | ADAR1 on target: 26.50% |
| ID | guide1_1_ | GAGTAGTGCCGT | (GCUG-GUCG) | ADAR2 on target: 97.89% |
| NO: | Glu2bQR_ | AGTGTCGAATCT | 1/1 A/C mismatch at 0 position | ADAR1 specificity: 1.13 |
| 138 | 512_ | TTGCAATGA | 1/1 U/G wobble base pair at +4 position | ADAR2 specificity: 0.94 |
| | gID_ | | 1/1 G/U wobble base pair at +6 position | |
| | 07129_ | | 1/1 A/G mismatch at +13 position | |
| | v0022 | | | |
| SEQ | LRRK1_ | TACAGCAGTACT | 1/1 A/C mismatch at 0 position | ADAR1 on target: 76.01% |
| ID | guide10_ | GAGTGGTGCCGT | 1/1 U/G wobble base pair at +4 position | ADAR2 on target: 86.64% |
| NO: | Glu2bQR_ | AGTCAGCAATCT | 1/1 U/G wobble base pair at +5 position | ADAR1 specificity: 1.18 |
| 139 | 512_ | TTGCAATGA | 1/1 G/U wobble base pair at +6 position | ADAR2 specificity: 1.14 |
| | gID_ | | | |
| | 06733_ | | | |
| | v0414 | | | |
| SEQ | LRRK1_ | TACAGCAGGACT | 1/1 A/C mismatch at 0 position | ADAR1 on target: 65.42% |
| ID | guide10_ | GGGCGGTGCCGT | 1/1 U/G wobble base pair at +4 position | ADAR2 on target: 91.92% |
| NO: | Glu2bQR_ | AGTCAGCAATCT | 1/1 U/G wobble base pair at +5 position | ADAR1 specificity: 1.26 |
| 140 | 512_ | TTGCAATGA | 1/1 U/G wobble base pair at +8 position | ADAR2 specificity: 1.28 |
| | gID_ | | 1/1 A/G mismatch at +13 position | |
| | 06733_ | | | |
| | v0302 | | | |
| SEQ | LRRK1_ | TACAGCAGTCCT | 1/1 A/C mismatch at 0 position | ADAR1 on target: 67.04% |
| ID | guide10_ | GGGCGGTGCCGT | 1/1 U/G wobble base pair at +4 position | ADAR2 on target: 89.53% |
| NO: | Glu2bQR_ | AGTCAGCAATCT | 1/1 U/G wobble base pair at +5 position | ADAR1 specificity: 1.28 |
| 141 | 512_ | TTGCAATGA | 1/1 U/G wobble base pair at +8 position | ADAR2 specificity: 1.02 |
| | gID_ | | 1/1 U/C mismatch at +12 position | |
| | 06733_ | | | |
| | v0494 | | | |
| SEQ | LRRK1_ | TAAAGCAGTACT | 4/4 symmetric bulge at −6 position | ADAR1 on target: 38.94% |
| ID | guide11_ | GAGCAGTGCCGT | (GCUG-GUCG) | ADAR2 on target: 97.94% |
| NO: | Glu2bQR_ | AGTGTCGAATCT | 1/1 A/C mismatch at 0 position | ADAR1 specificity: 0.95 |
| 142 | 512_ | TTGCAATGA | 1/1 U/G wobble base pair at +4 position | ADAR2 specificity: 1.03 |
| | gID_ | | | |
| | 07129_ | | | |
| | v0134 | | | |
| SEQ | LRRK1_ | TAAAGCAGGACT | 4/4 symmetric bulge at −6 position | ADAR1 on target: 45.55% |
| ID | guide11_ | GAGCAGTGCCGT | (GCUG-GUCG) | ADAR2 on target: 99.01% |
| NO: | Glu2bQR_ | AGTGTCGAATCT | 1/1 A/C mismatch at 0 position | ADAR1 specificity: 1.18 |
| 143 | 512_ | TTGCAATGA | 1/1 U/G wobble base pair at +4 position | ADAR2 specificity: 0.92 |
| | gID_ | | 1/1 A/G mismatch at +13 position | |
| | 07129_ | | | |
| | v0006 | | | |
| SEQ | LRRK1_ | TACAGCAGGACT | 4/4 symmetric bulge at −6 position | ADAR1 on target: 55.62% |
| ID | guide11_ | GGGCAGTGCCGT | (GCUG-GUCG) | ADAR2 on target: 99.09% |
| NO: | Glu2bQR_ | AGTGTCGAATCT | 1/1 A/C mismatch at 0 position | ADAR1 specificity: 1.25 |
| 144 | 512_ | TTGCAATGA | 1/1 U/G wobble base pair at +4 position | ADAR2 specificity: 0.89 |
| | gID_ | | 1/1 U/G wobble base pair at +8 position | |
| | 07129_ | | 1/1 A/G mismatch at +13 position | |
| | v0294 | | | |

### Example 10

### ABCA4-Targeting Engineered Guide RNAs

This example describes engineered guide RNAs of the present disclosure targeting the ABCA4 G1961E mutation, an ABCA4 missense mutation, implicated in Stargardt disease. The G1961E mutation includes a 5′G upstream of the A to be edited. This editing context is especially difficult to target as it can be refractory to endogenous ADAR editing.

### High Throughput Screening

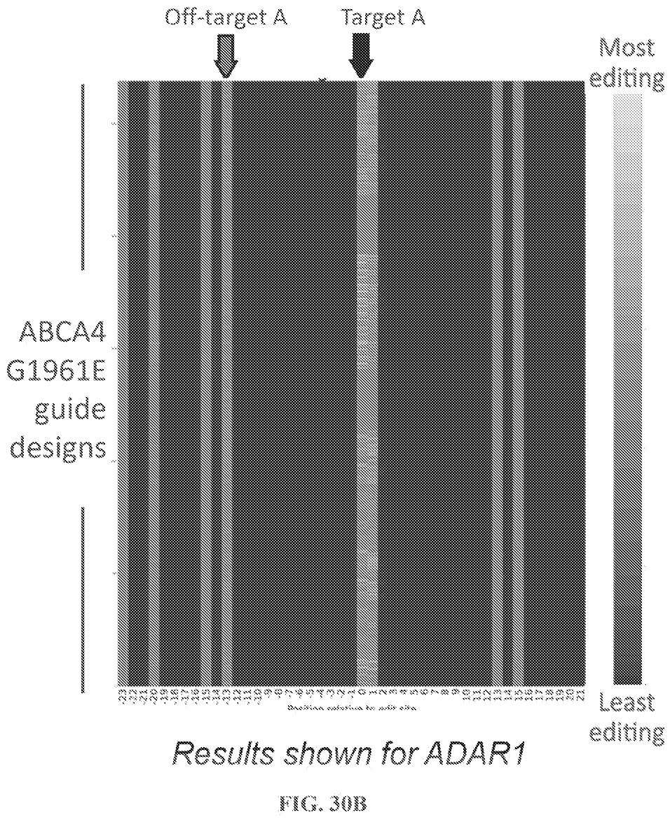

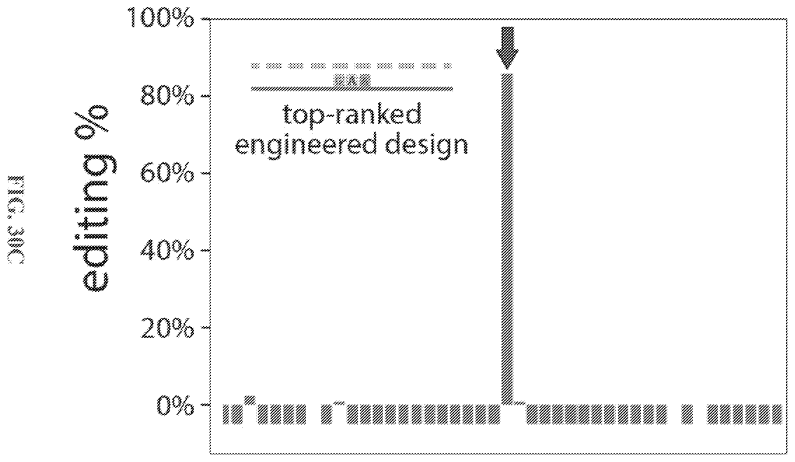

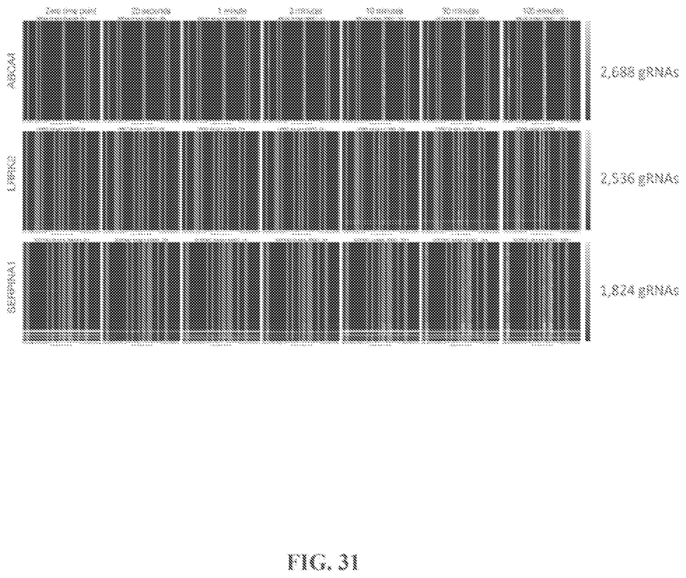

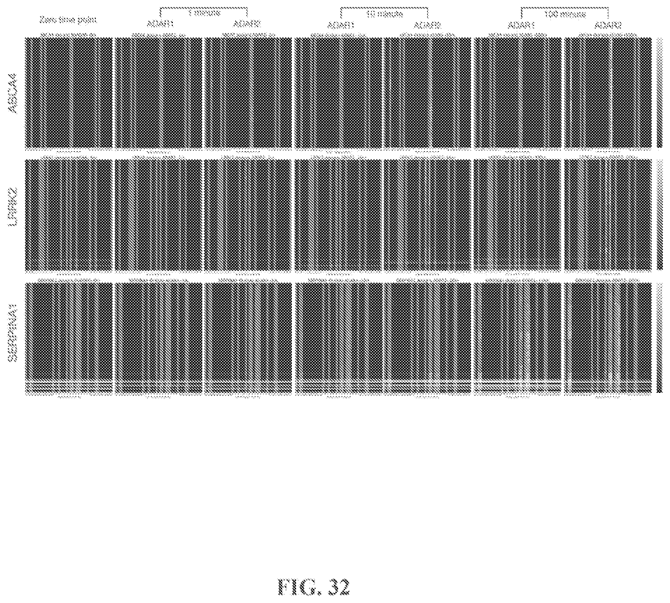

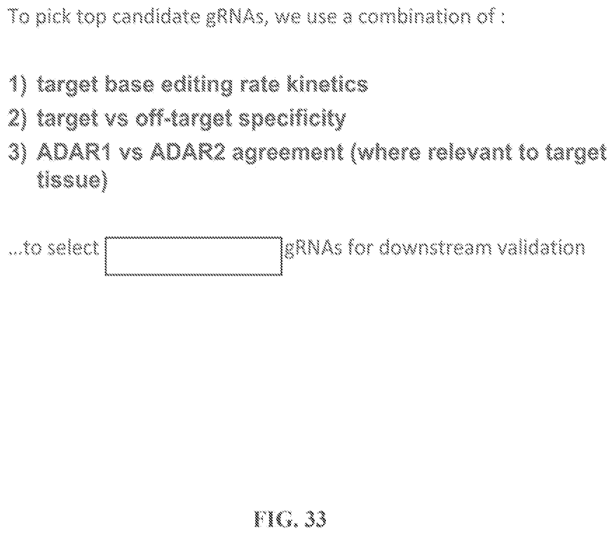

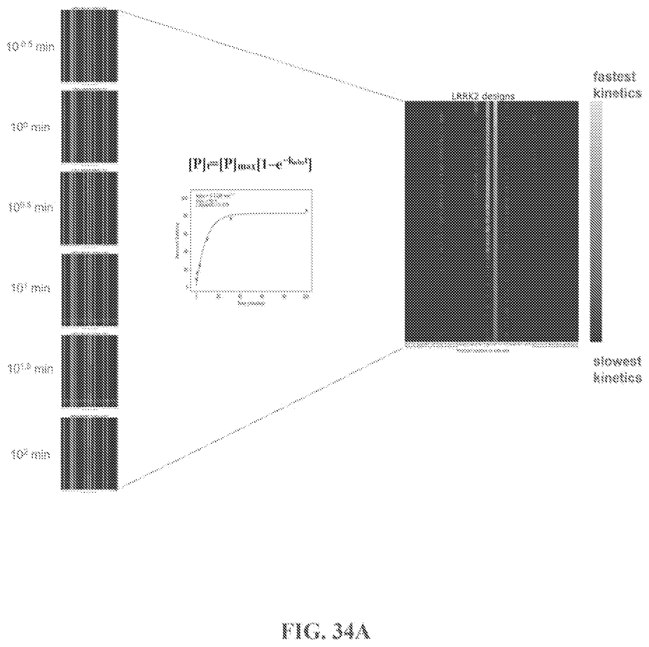

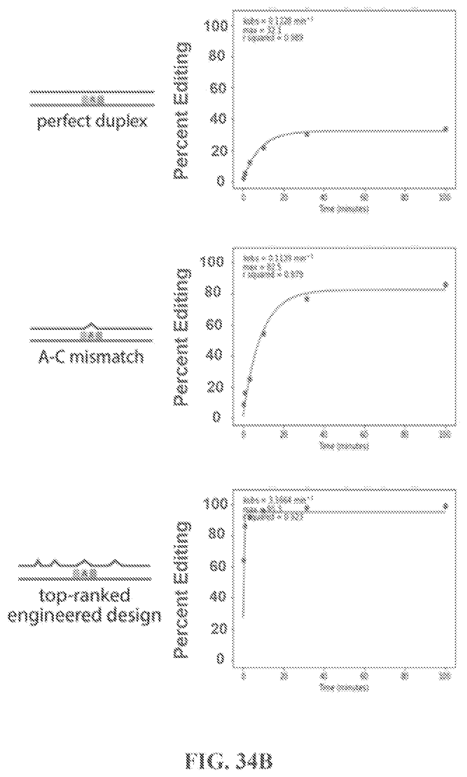

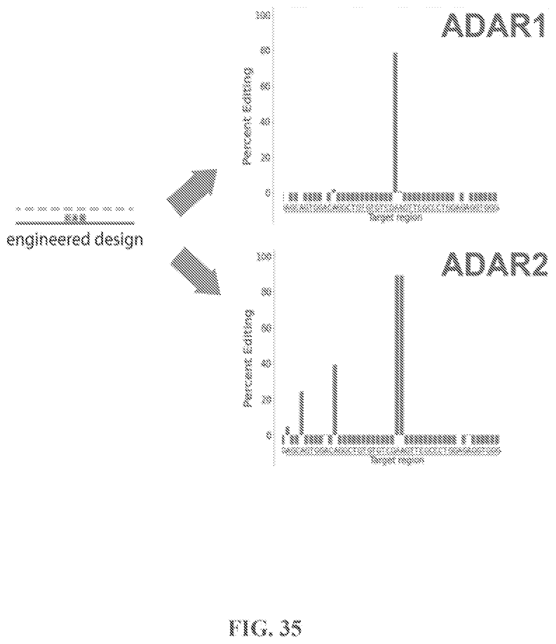

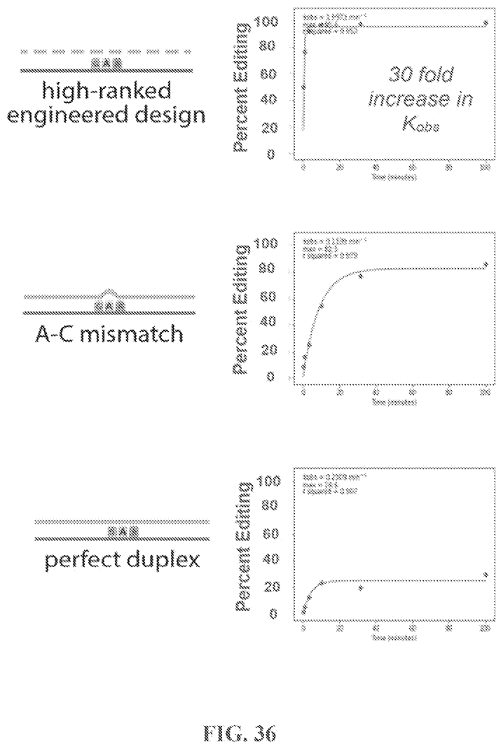

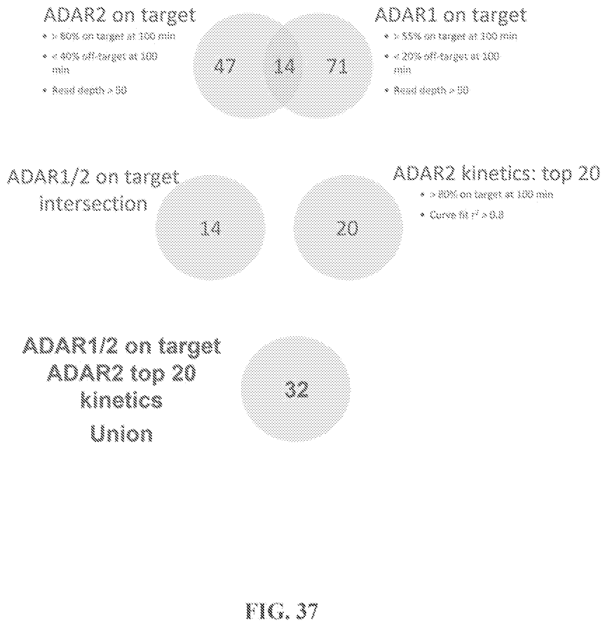

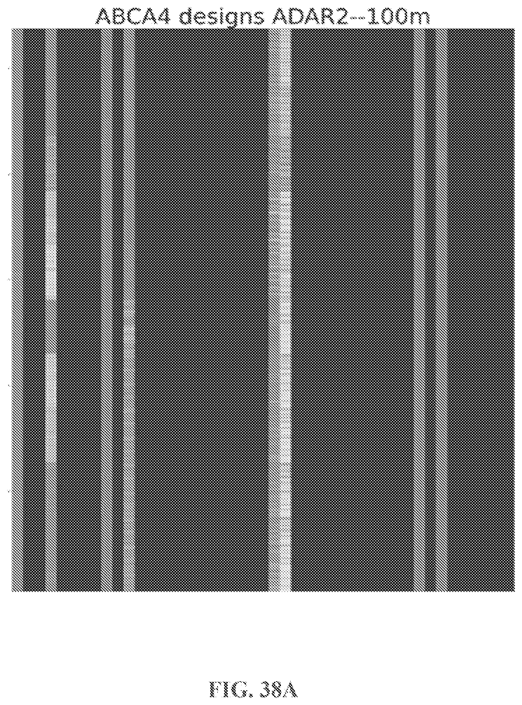

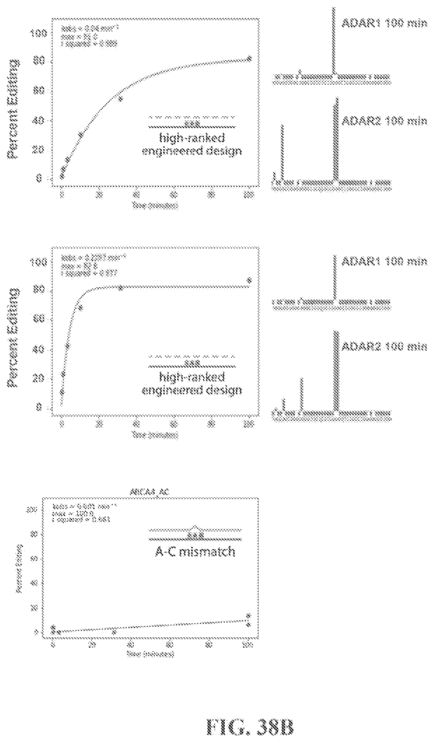

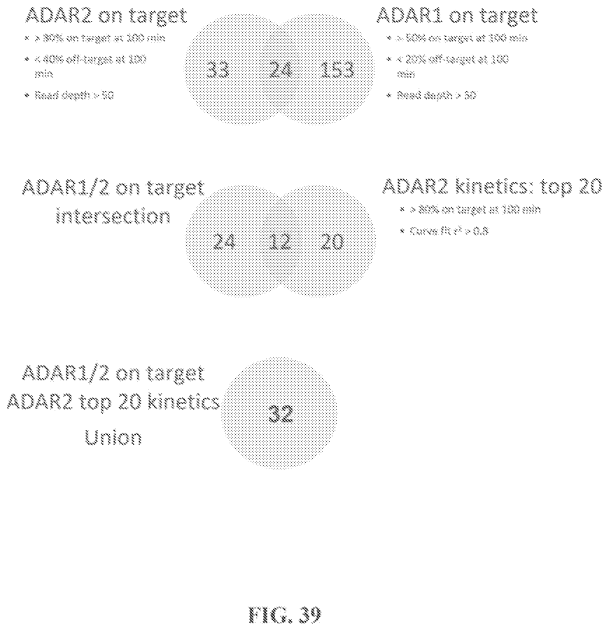

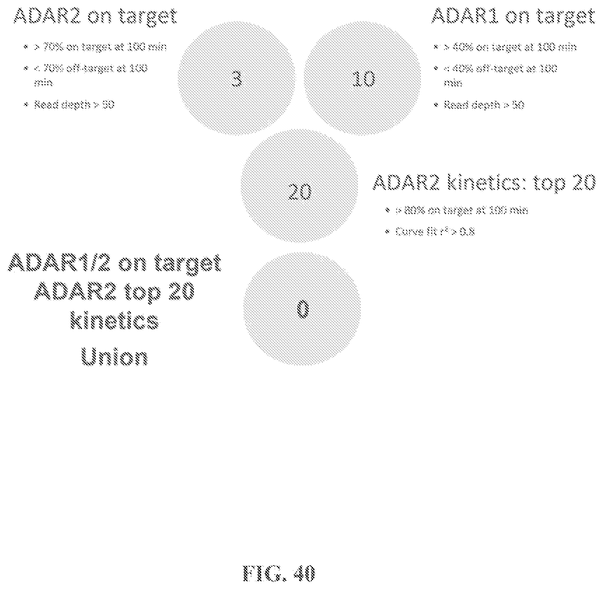

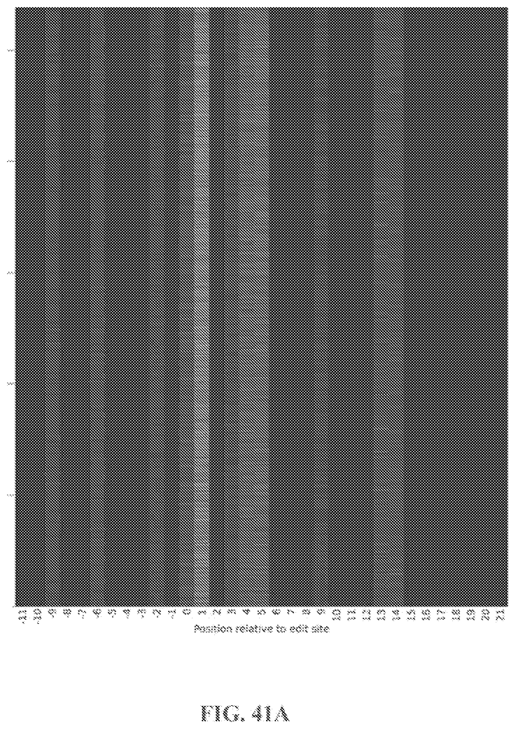

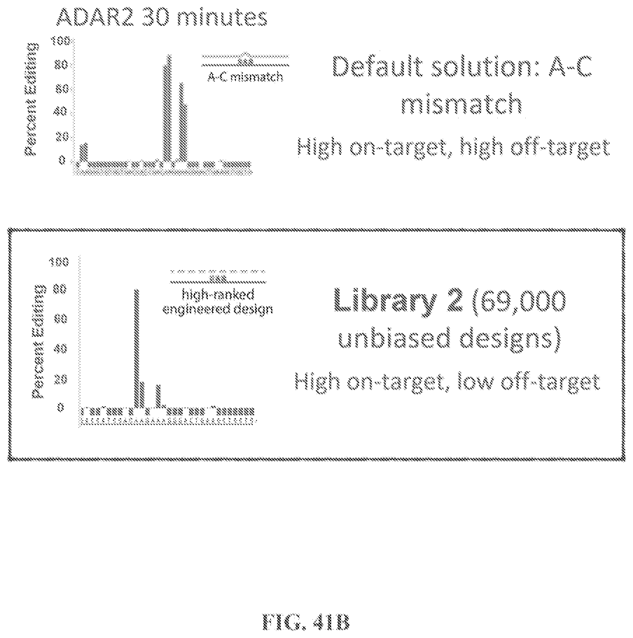

High throughput screening (HTS) of 2,500 guide designs were generated and screen for targeting the ABCA4 G1961E mutation, as shown in FIG. 30A-FIG. 30C. In FIG. 30A-FIG. 30C, experiments included incubation of engineered guide RNAs with ADAR1 for 100 min followed by an NGS readout of editing efficiency. FIG. 30A shows percent editing of the on-target A (indicated by the arrow) and any off-target editing 5′ and 3′ of the target A to be edited. With both the engineered guide RNA that forms a perfect duplex with the target ABCA4 RNA (top graph) and the engineered guide RNA that forms a single A/C mismatch with the target ABCA4 RNA at the target A to be edited (bottom graph), on-target editing was negligible. FIG. 30B shows a heatmap of ADAR1-mediated RNA editing of the on-target A (position 0 on the x-axis) and off-target editing 5′ and 3′ of the target A to be edited. The y-axis indicates a unique engineered guide RNA against ABCA4. FIG. 30C shows percent editing of the on-target A (indicated by the arrow) and any off-target editing 5′ and 3′ of the target A to be edited for the top ranked engineered guide RNA (SEQ ID NO: 291) against ABCA4, which formed structural features, including a 1/1 G/G mismatch at the −1 position (relative to the target adenosine), a 1/1 U/U mismatch at the 3 position (relative to the target adenosine), and a 1/1 G/G mismatch at the 19 position (relative to the target adenosine), upon hybridization to the target ABCA4 RNA. As demonstrated in FIG. 30C, an engineered guide RNA having the structural features of X, Y, Z upon hybridization of to the target ABCA4 RNA exhibited high on-target A editing and negligible off-target editing.

Self-annealing RNA structures comprising the engineered polynucleotide sequences of TABLE 18 and the sequences of the regions targeted by the guide RNAs were contacted with ADAR1 and/or ADAR2 under conditions that allow for the editing of the regions targeted by the guide RNAs (“in cis-editing”). The regions targeted by the guide RNAs were subsequently assessed for editing using next generation sequencing (NGS).

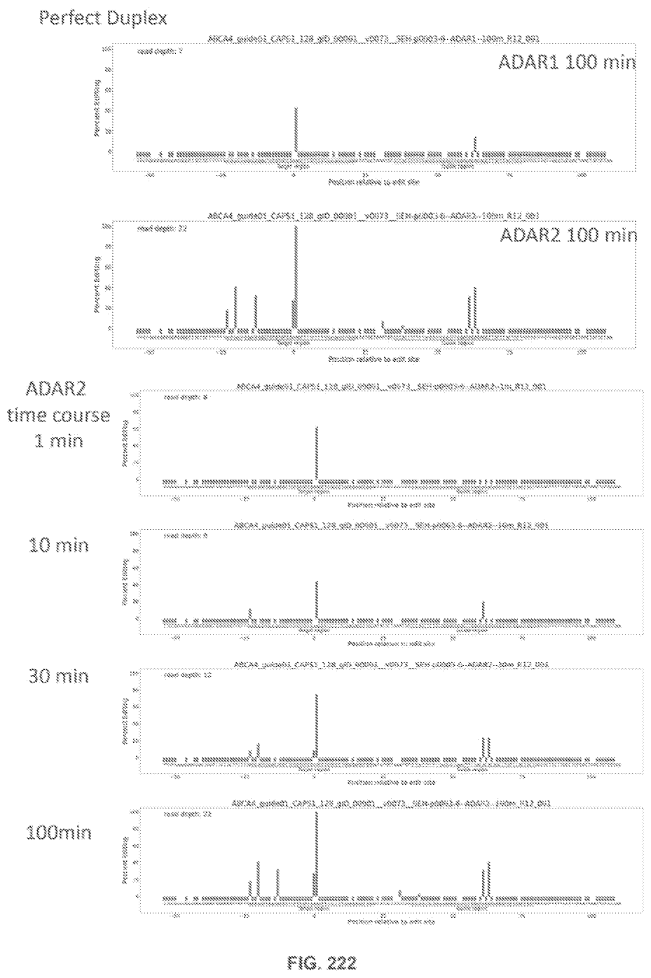

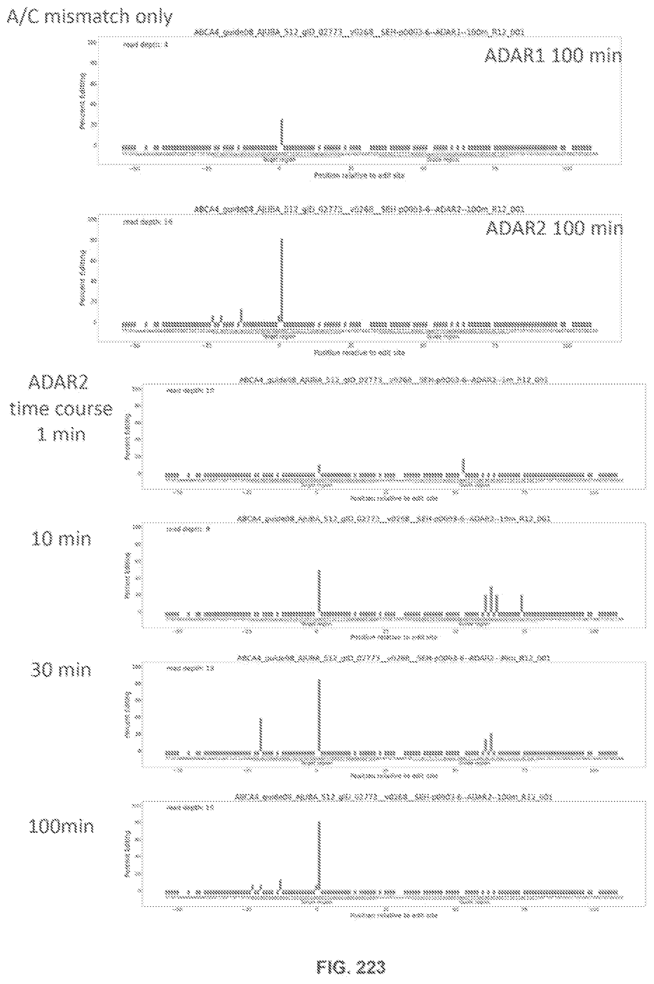

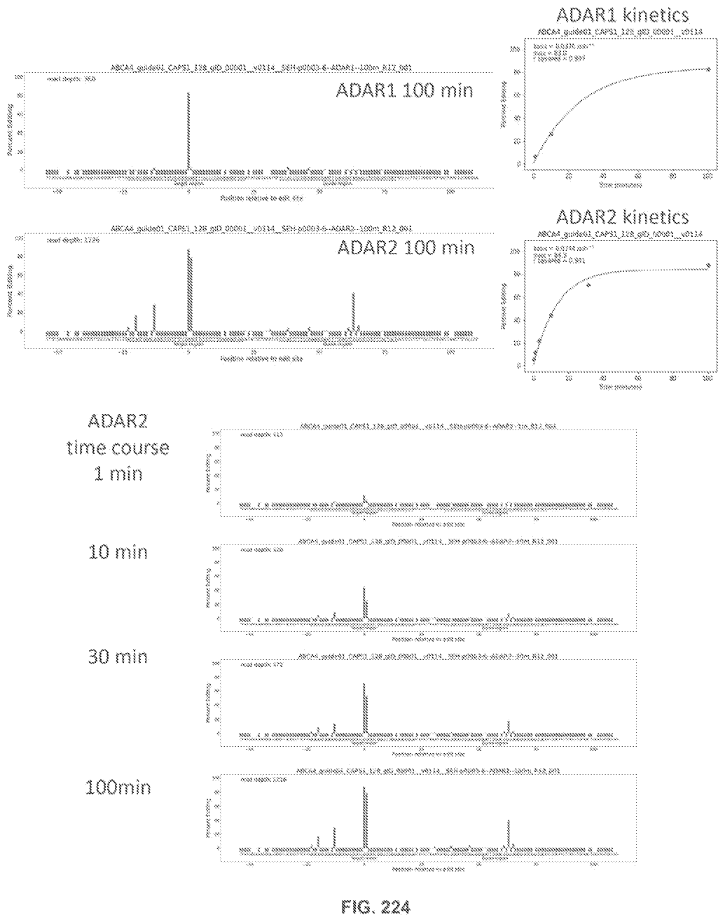

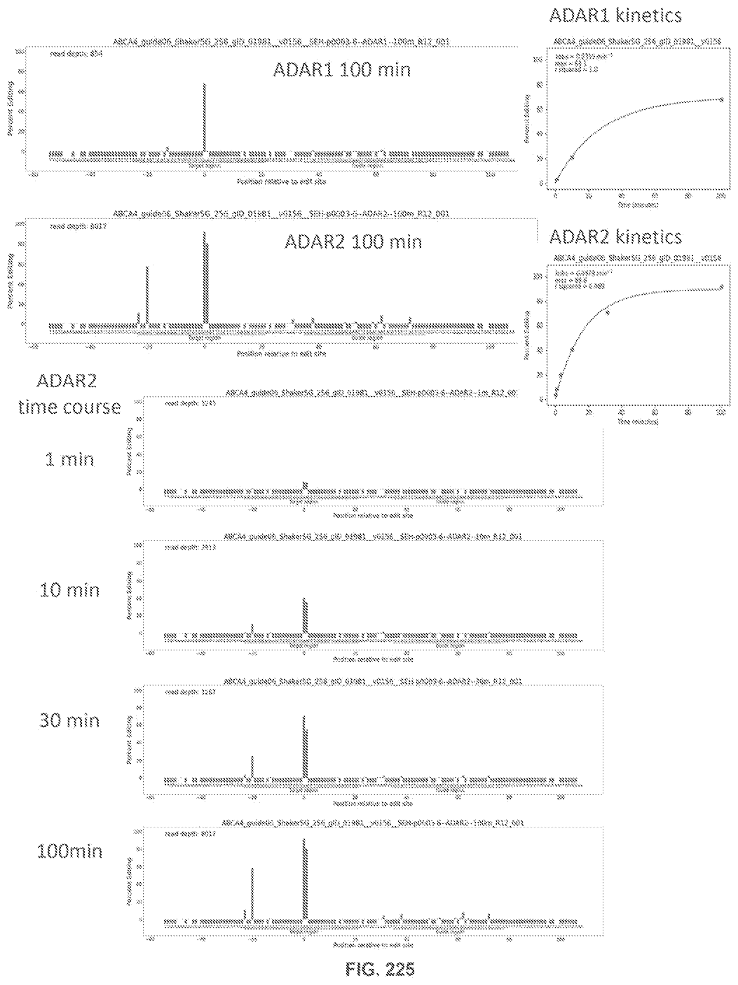

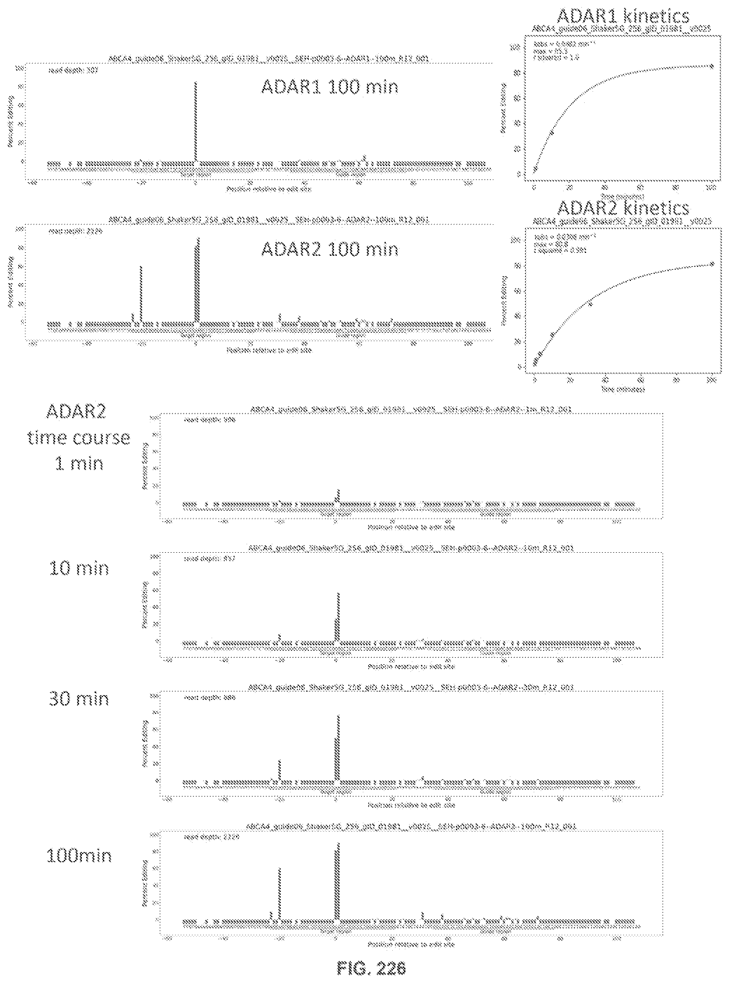

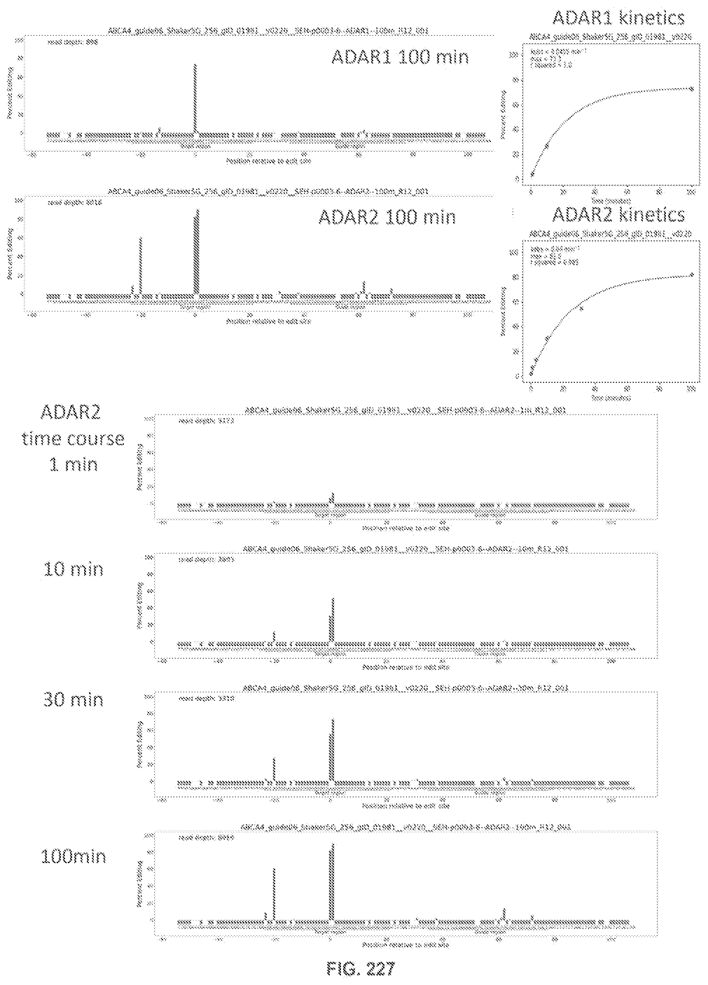

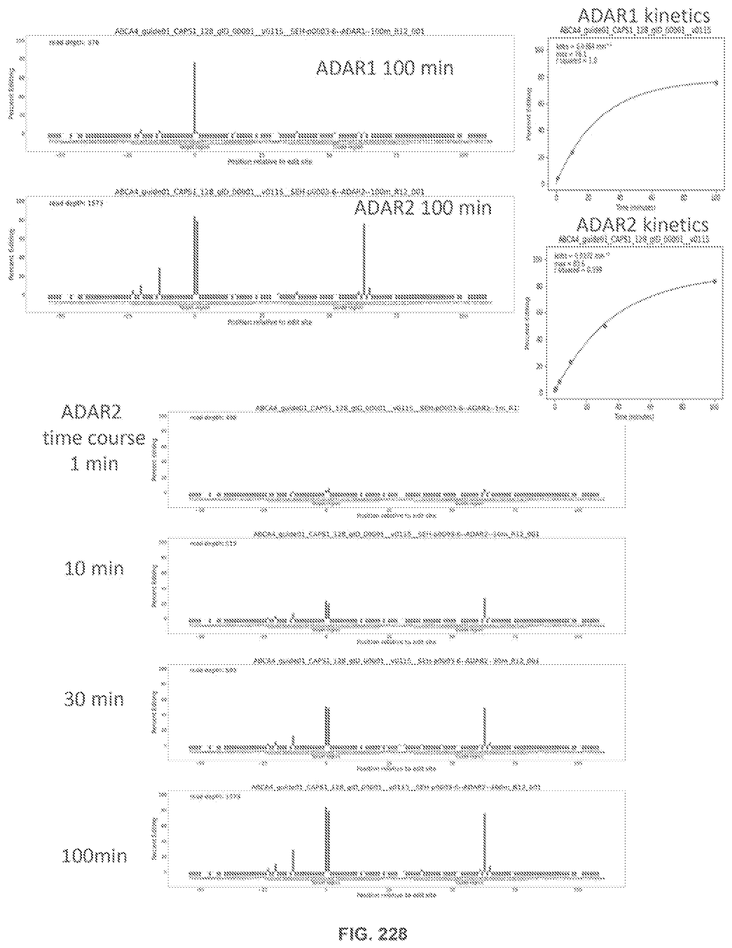

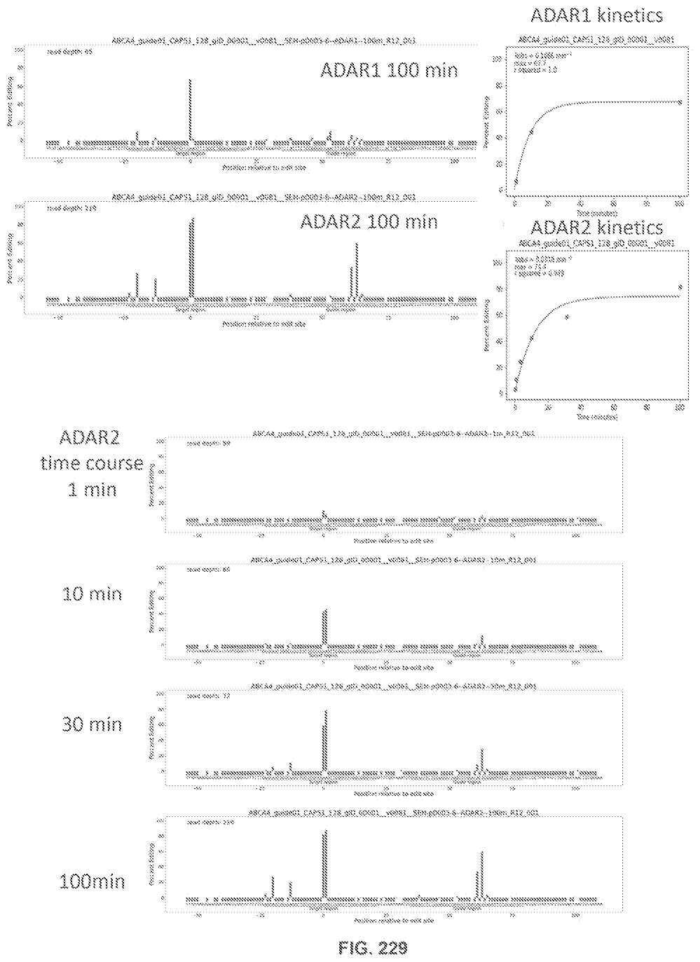

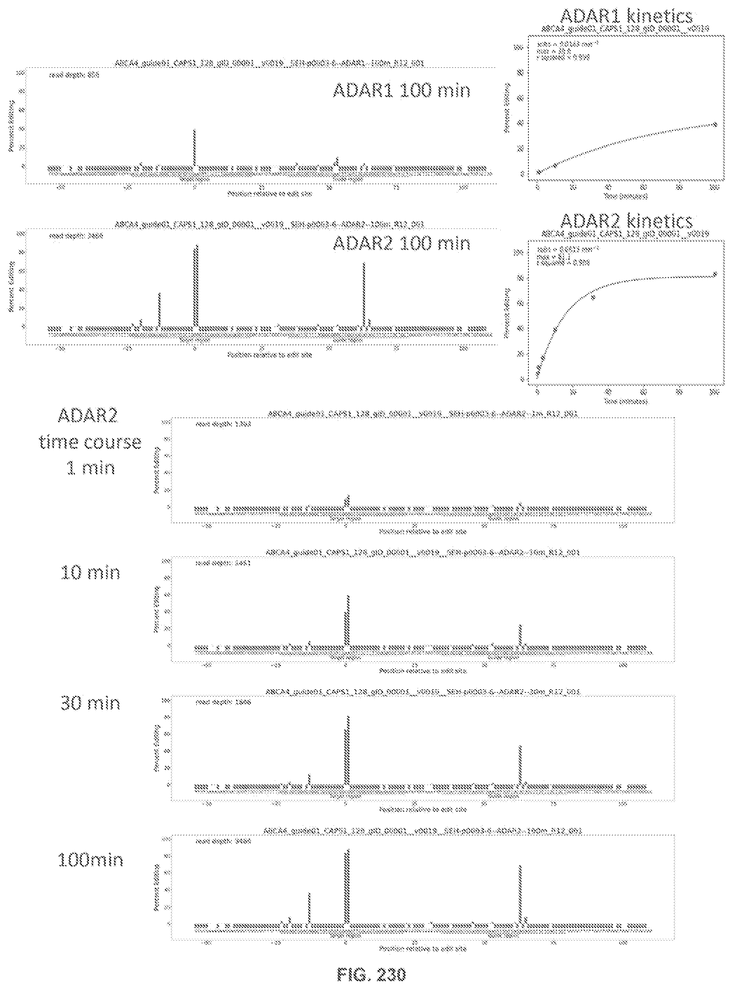

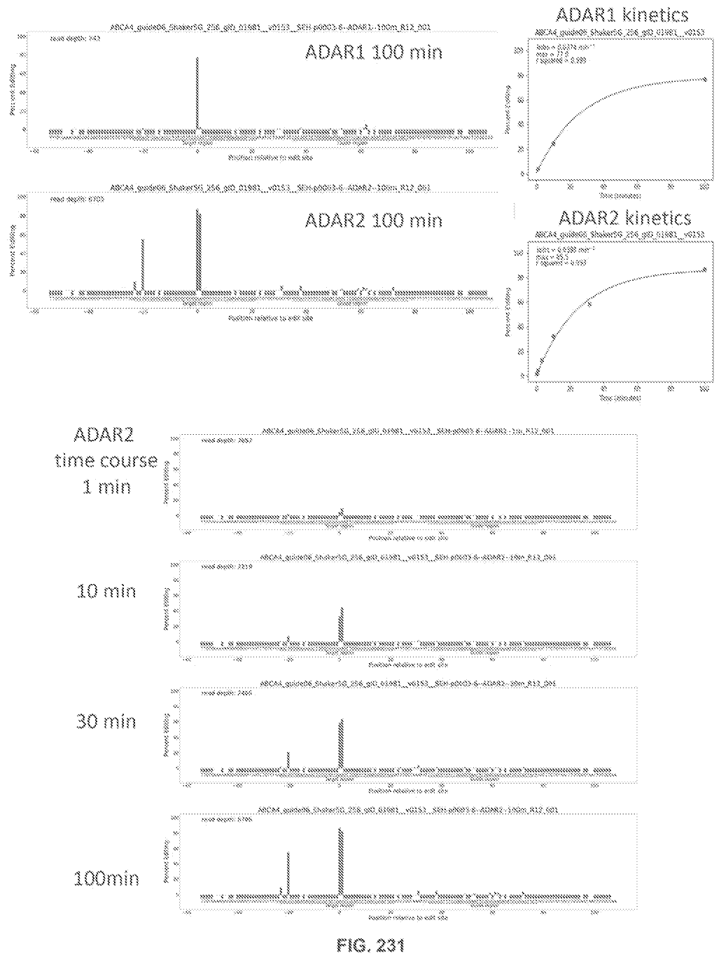

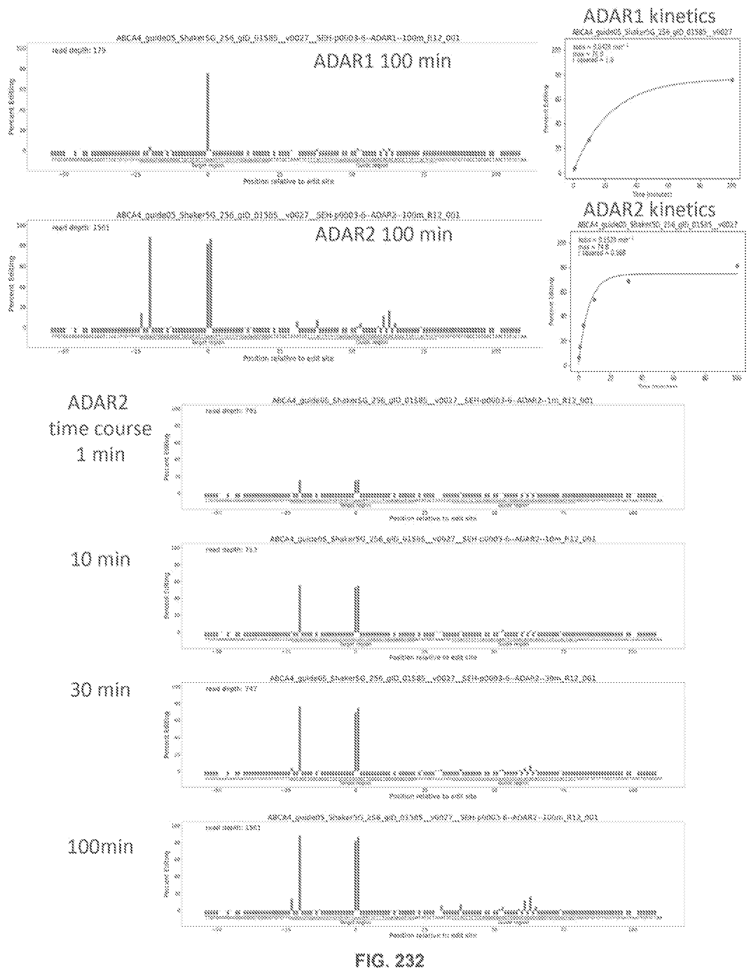

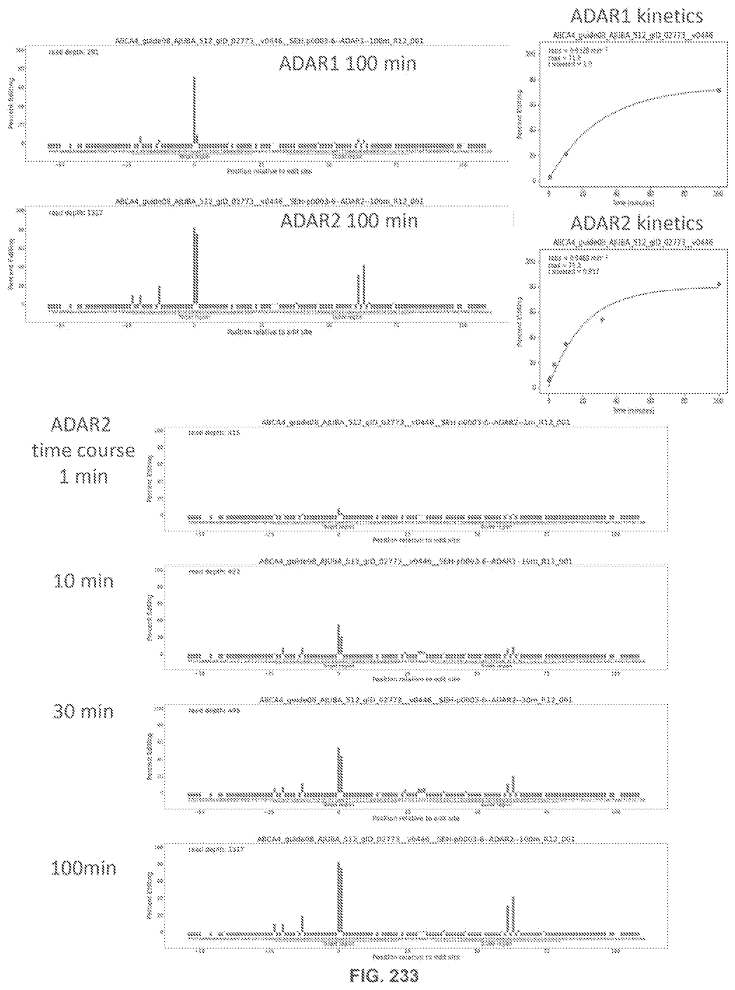

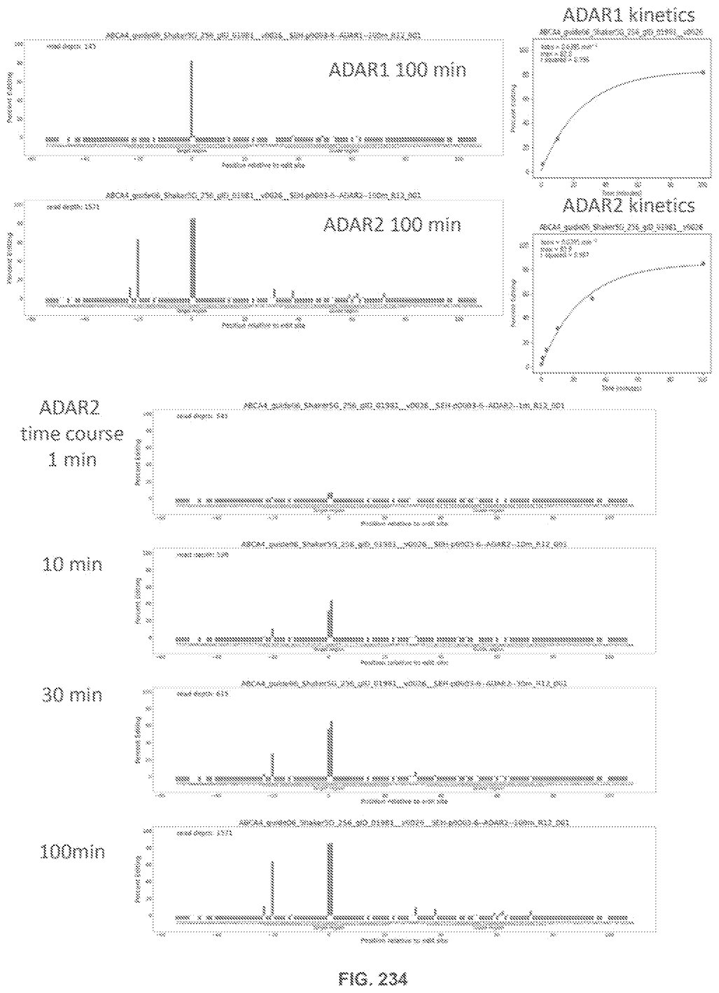

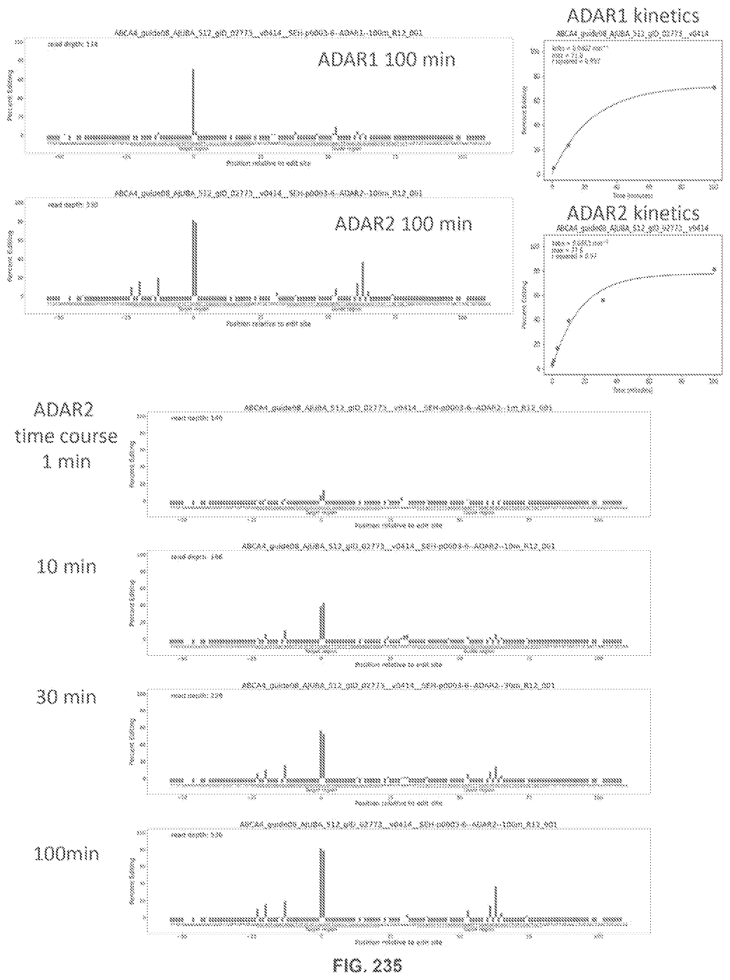

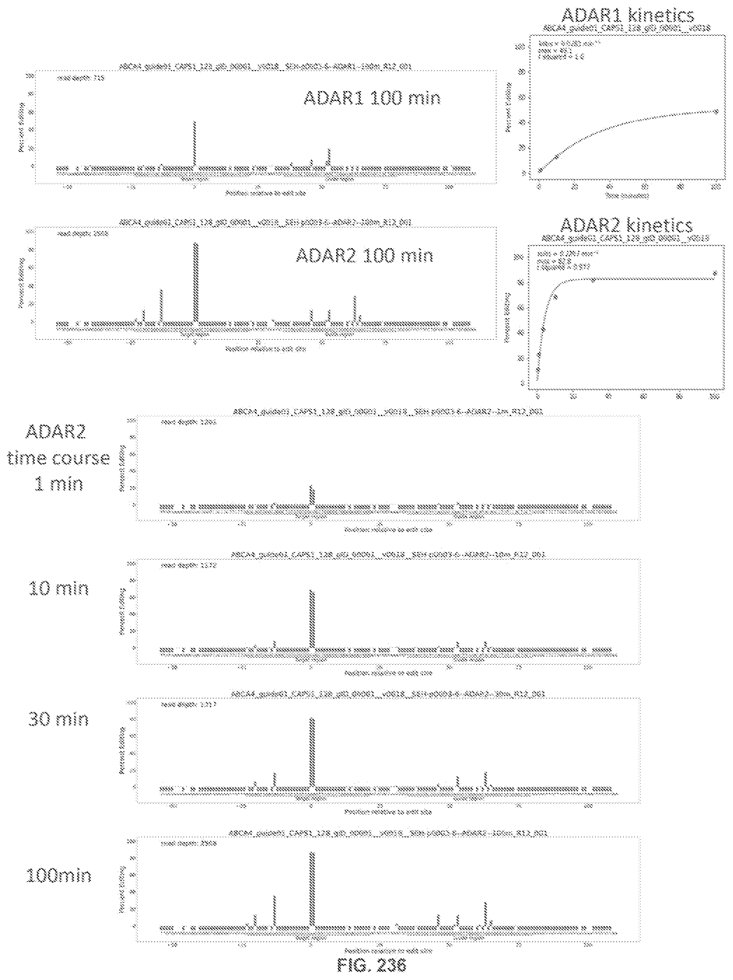

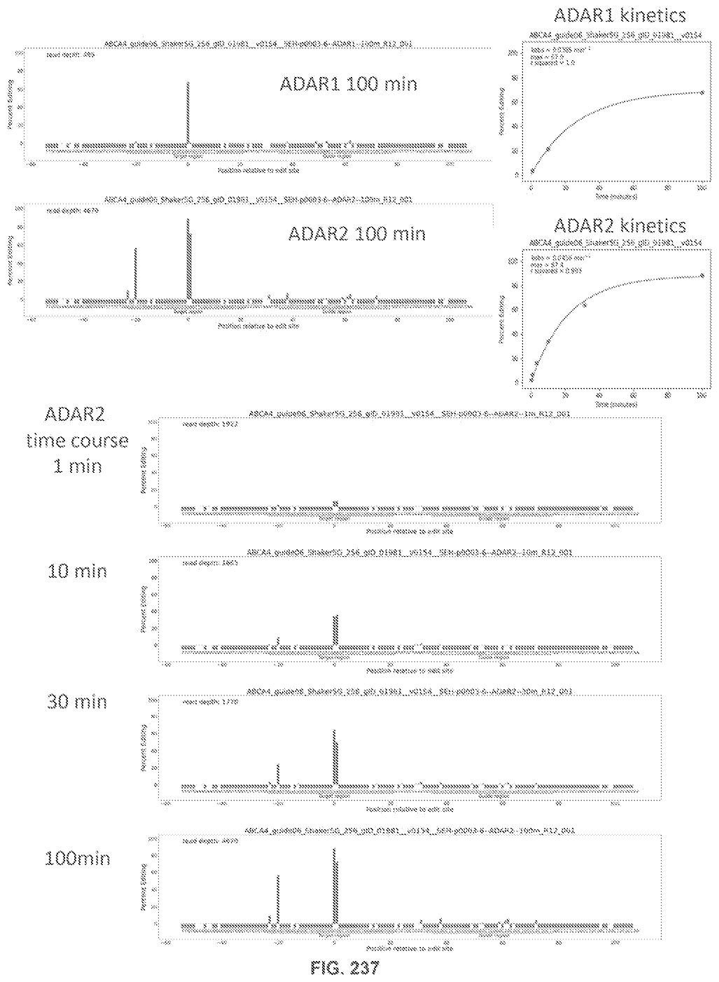

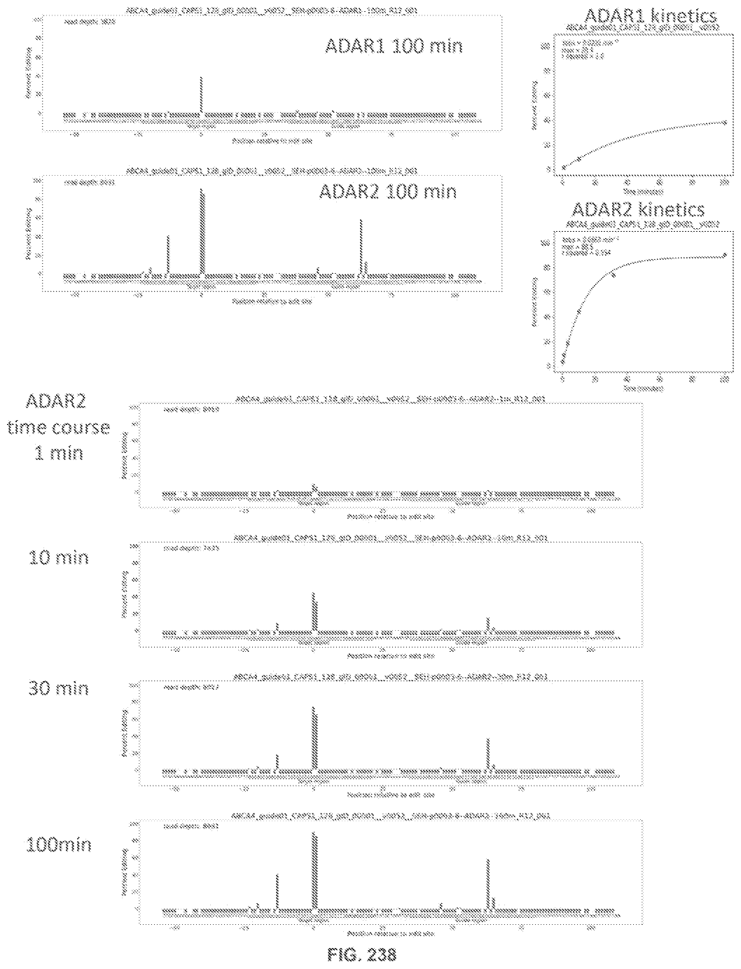

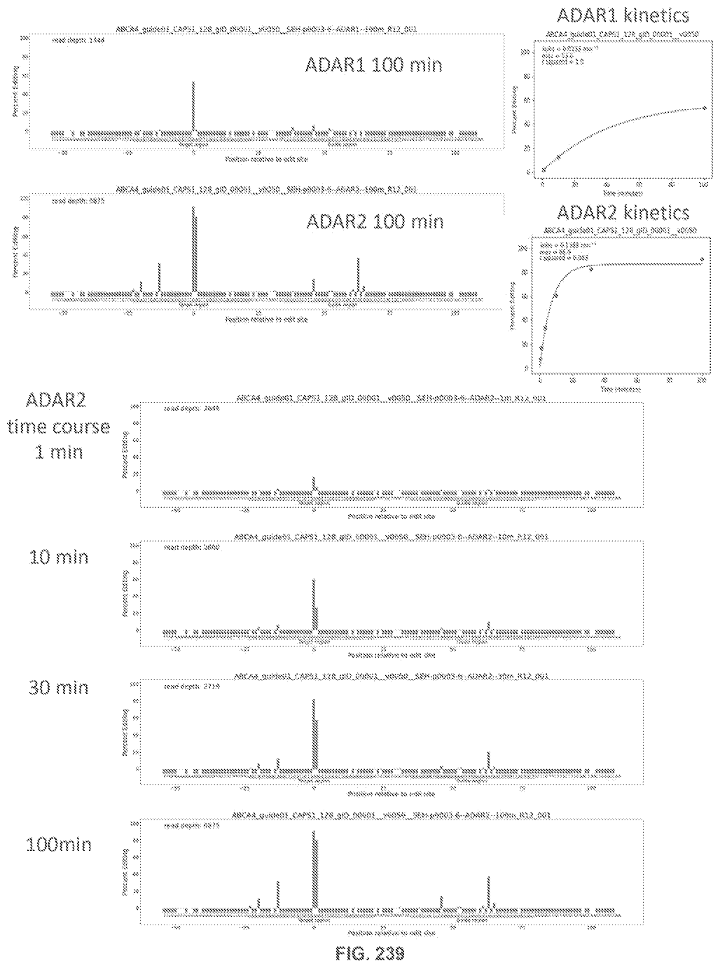

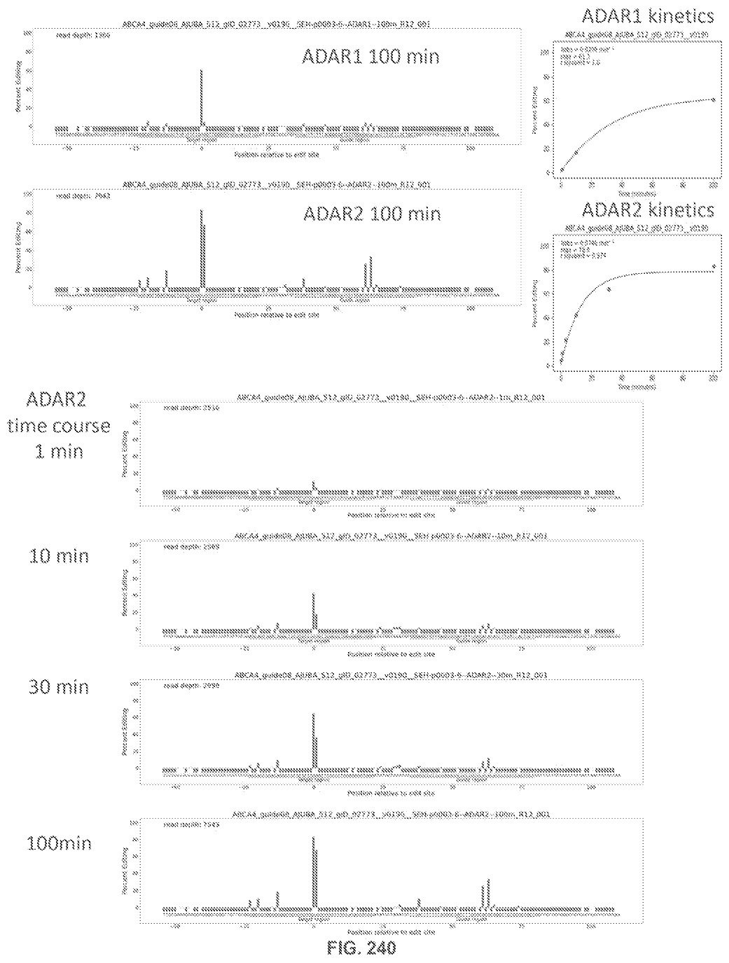

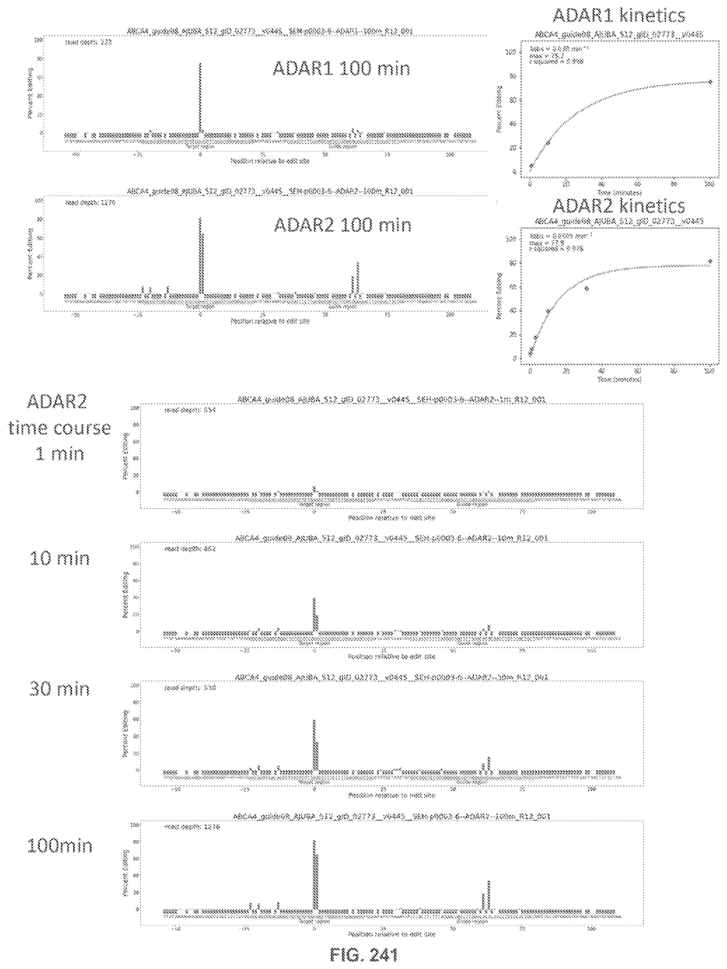

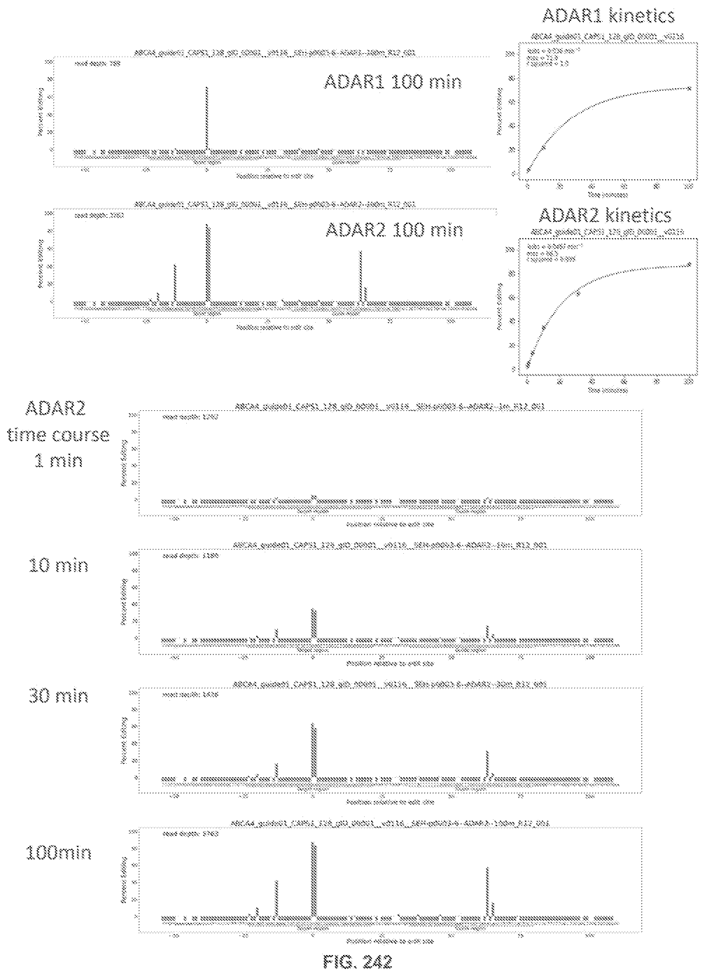

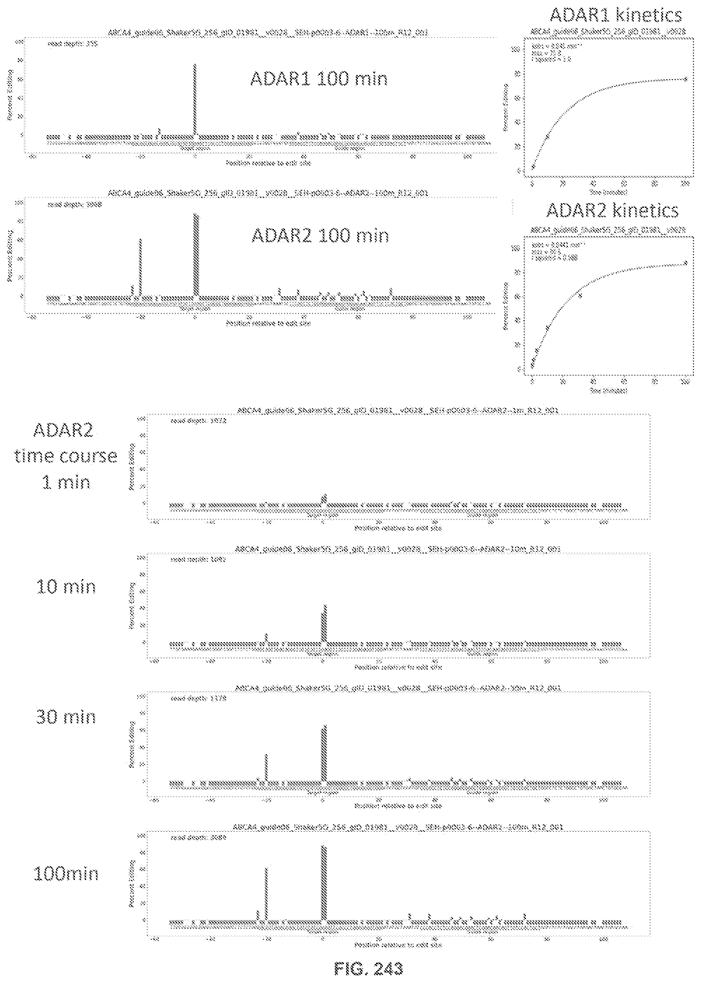

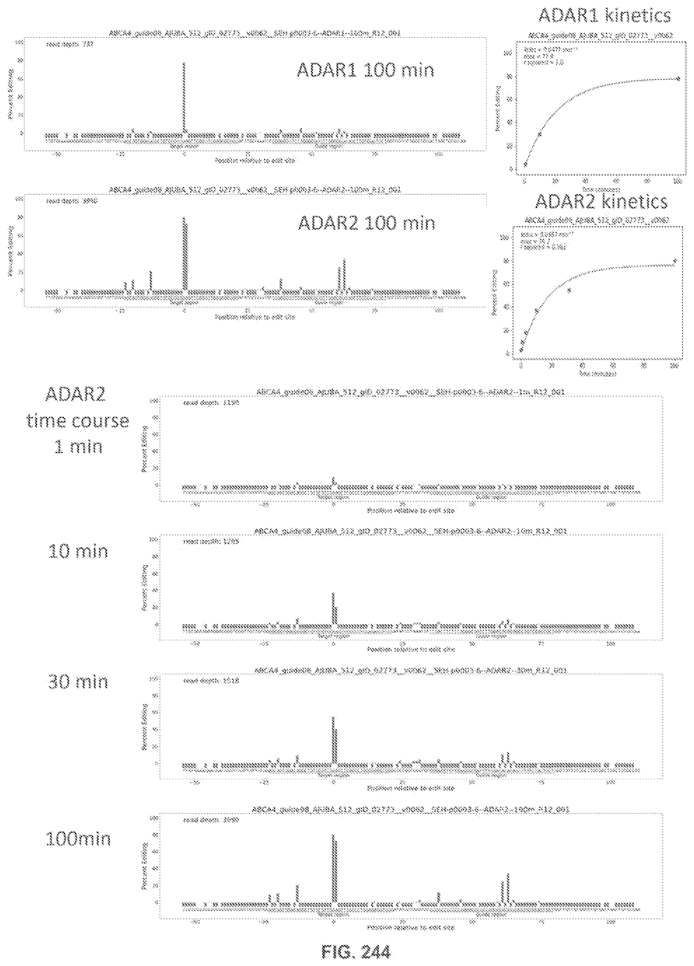

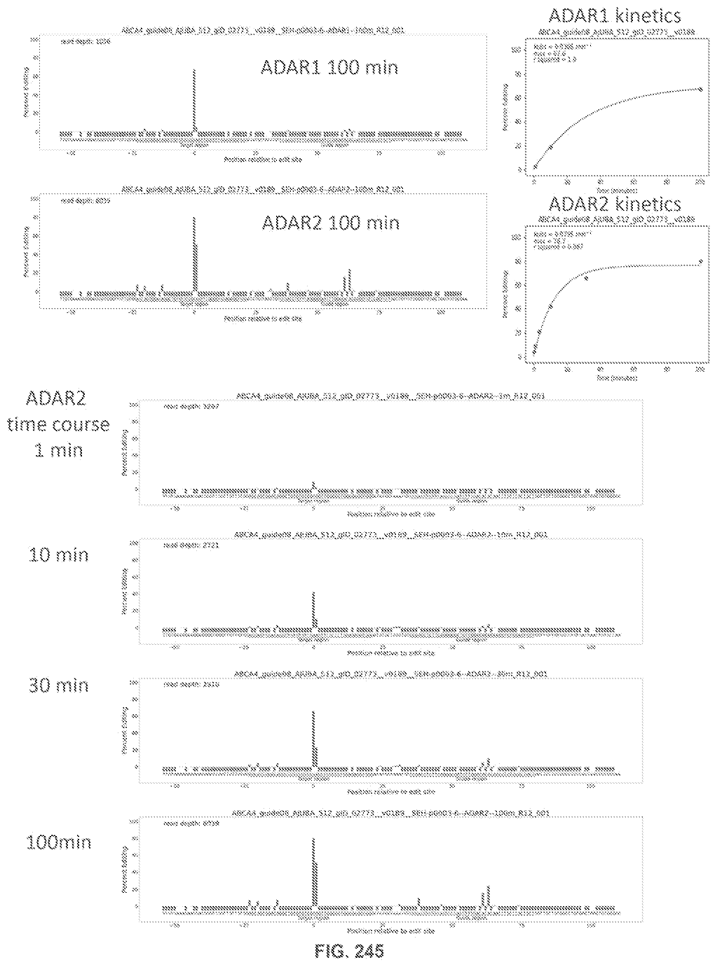

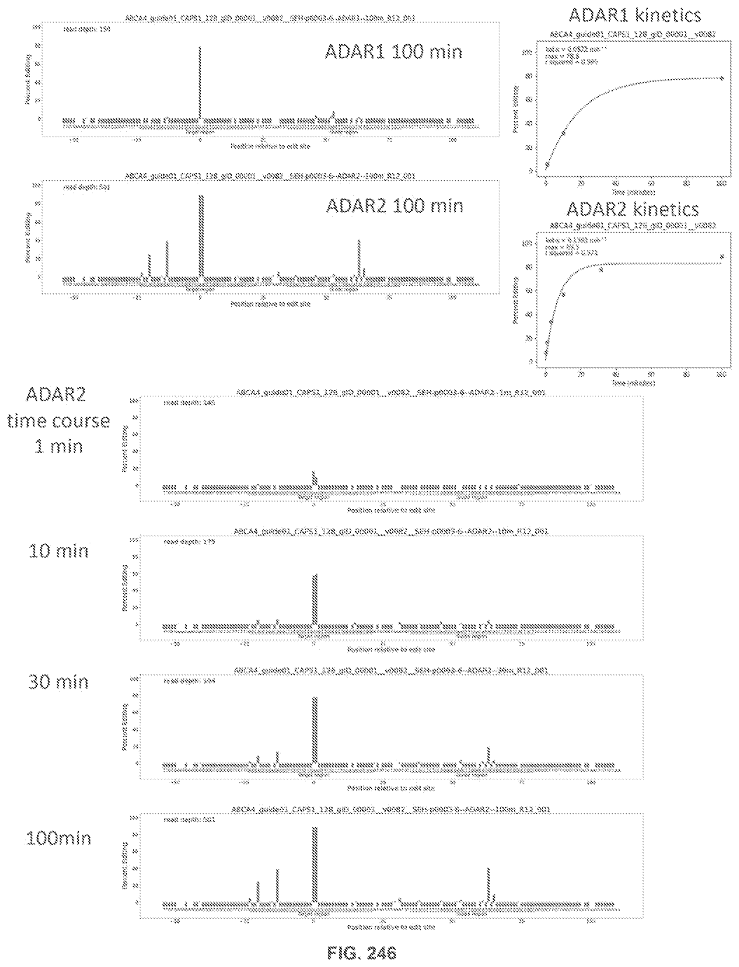

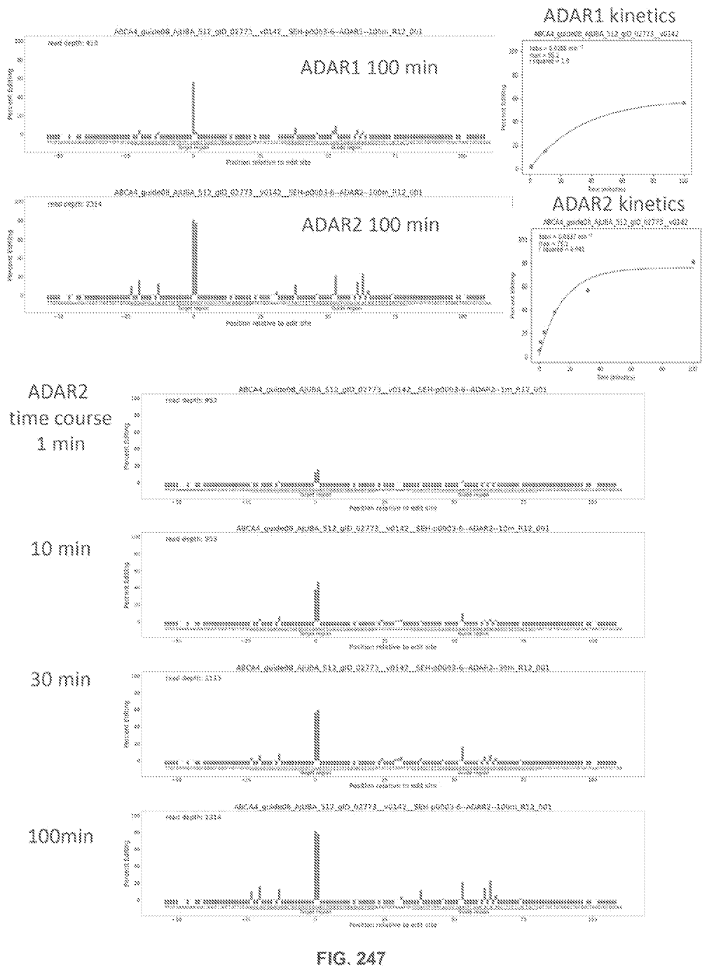

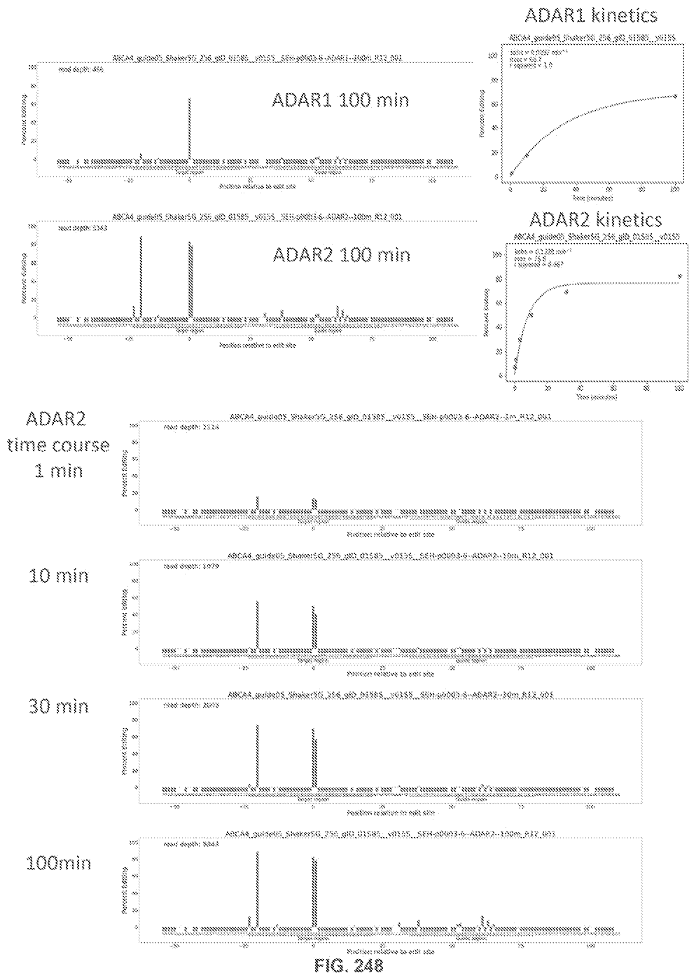

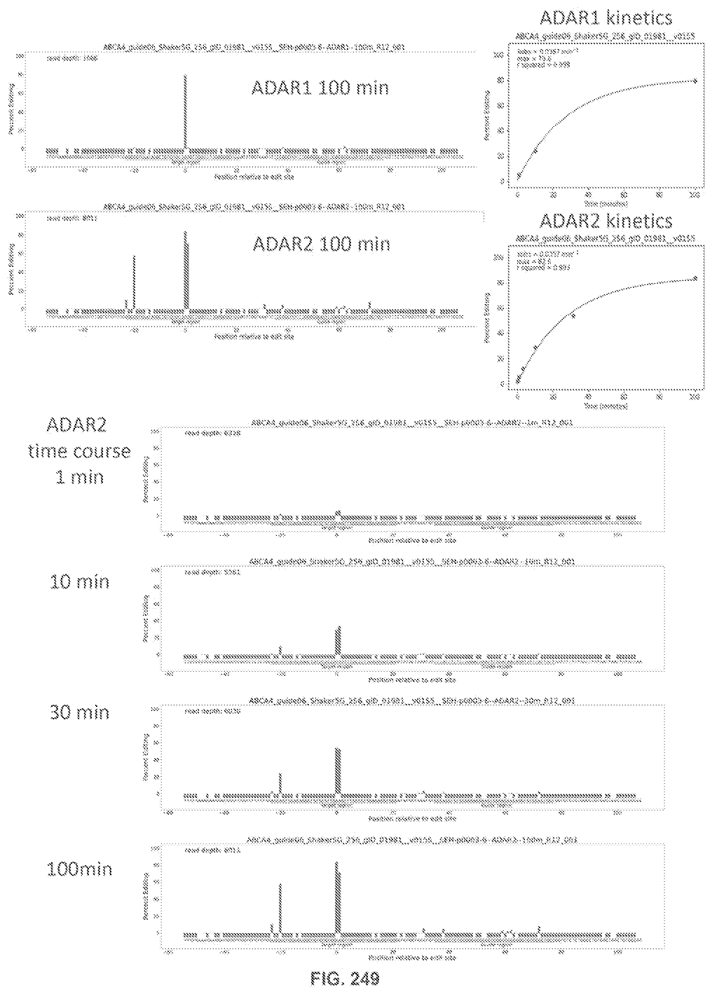

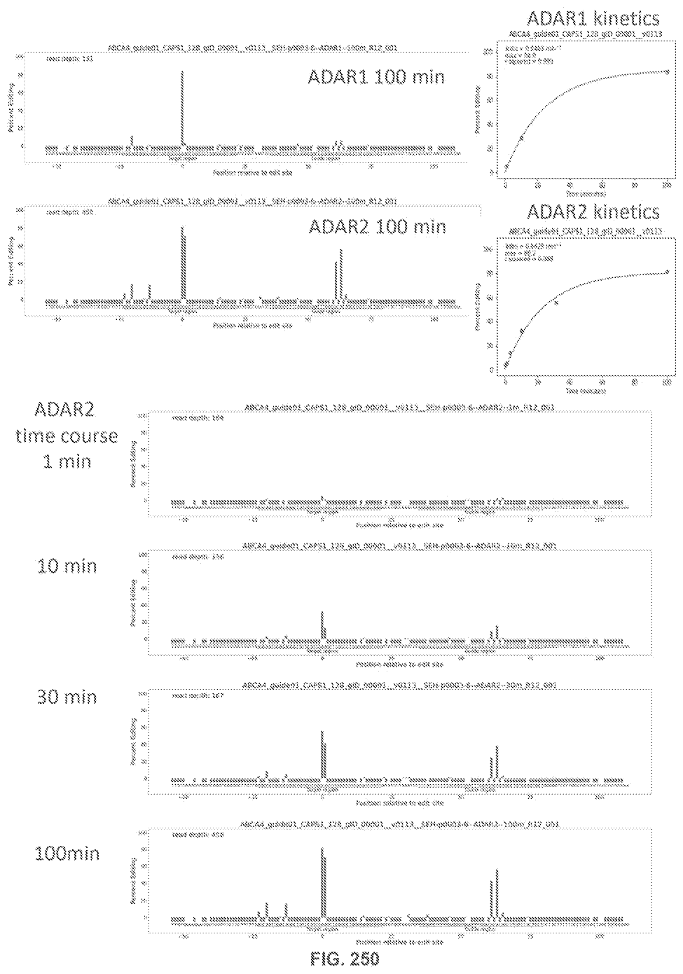

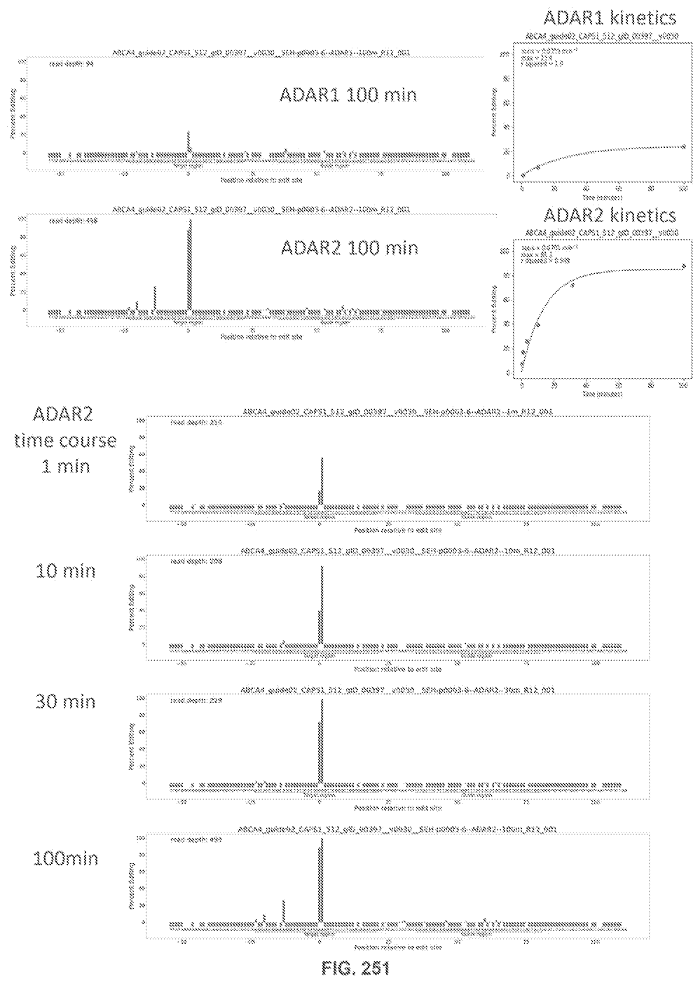

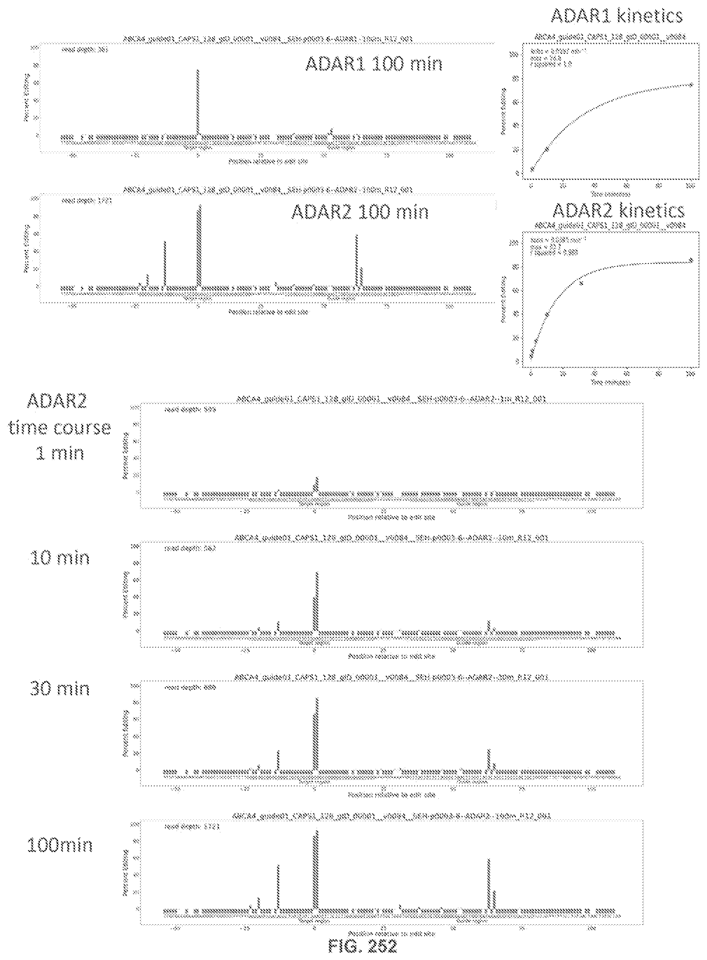

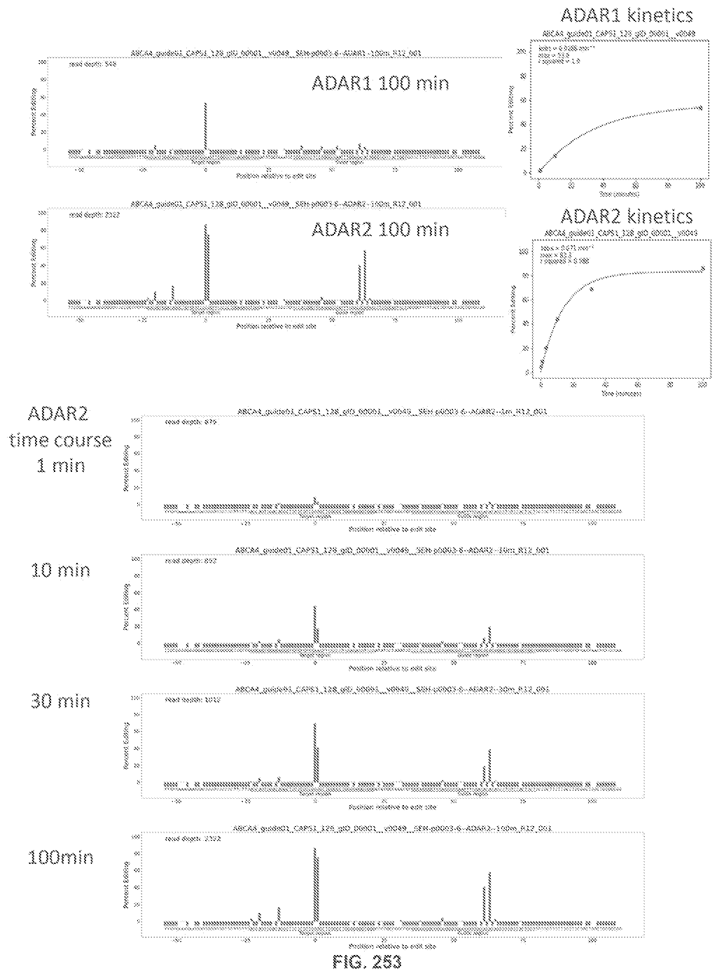

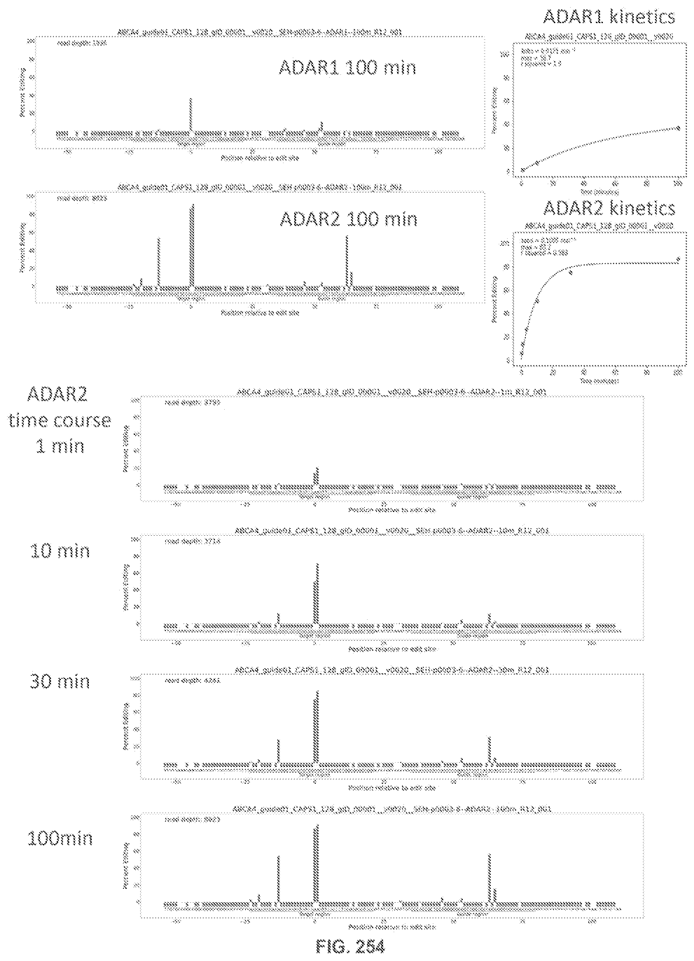

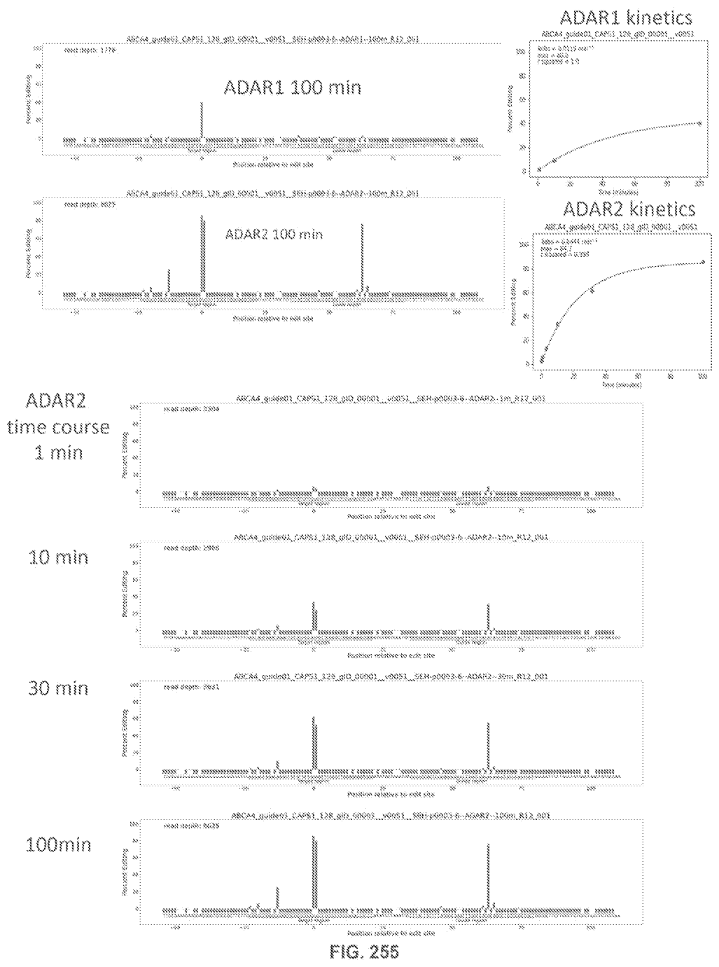

Shown in FIG. 218-FIG. 221 are structures of the self-annealing RNA structures comprising the engineered polynucleotide sequences of TABLE 18 and the sequence of the ABCA4 region targeted by the guide RNAs, highlighting the structural features summarized in TABLE 18. The target A was positioned towards the center of the guide-target RNA scaffold. FIG. 224-FIG. 255 show ADAR1 and ADAR2 on-target and off-target editing at the 100 min timepoint, ADAR1 and ADAR2 kinetics, and a timecourse of ADAR2 on-target and off-target editing at 1 min, 10 min, 30 min, and 100 min. Controls include a perfect duplex double stranded RNA substrate between the target sequence and the guide RNA (FIG. 222) and an engineered guide RNA that forms a single A/C mismatch upon hybridization to the target sequence (FIG. 223). Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 18
ABCA4 Engineered Guide RNAs, 45mers for In Cis-Editing Experiments

| SEQ | | | | |
|---|---|---|---|---|
| ID NO | | | Structural Features | |
| FIG | Name | Sequence | (target/guide) | Metrics |
| SEQ | Perfect | CCCACCTCTCCAGG | | ADAR1 on target: |
| ID | Duplex | GCGAACTTCGACAC | | 0.00% |
| NO: | | ACAGCCTGTCCACT | | ADAR2 on target: |
| 342 | | GCT | | 27.27% |
| FIG. | | | | ADAR1 specificity: 1 |
| 222 | | | | ADAR2 specificity: |
| | | | | 0.97 |
| SEQ | A/C | CCCACCTCTCCAGG | 1/1 A/C mismatch at 0 | ADAR1 on target: |
| ID | Mismatch | GCGAACTCCGACAC | position | 33.33% |
| NO: | | ACAGCCTGTCCACT | | ADAR2 on target: |
| 343 | | GCT | | 13.33% |
| FIG. | | | | ADAR1 specificity: |
| 223 | | | | 1.33 |
| | | | | ADAR2 specificity: |
| | | | | 1.06 |
| SEQ | >ABCA4_ | CCCACCTCTCCAGG | 1/1 G/U wobble base pair at | ADAR1 on target: |
| ID | guide01_ | GCGAATTTGGACAT | −6 position | 83.06% |
| NO: | CAPS1_128_ | ACAGCCTGTCCACT | 1/1 G/G mismatch at −1 | ADAR2 on target: |
| 218 | gID_ | GCT | position | 88.09% |
| FIG. | 00001_v0114 | | 1/1 G/U wobble base pair at | ADAR1 specificity: |
| 218 | | | +2 position | 1.79 |
| FIG. | | | | ADAR2 specificity: |
| 224 | | | | 1.46 |
| SEQ | >ABCA4_ | CCCACCTCTCCGGG | 1/1 G/U wobble base pair at | ADAR1 on target: |
| ID | guide06_ | ACGAACTCGGACA | −12 position | 68.15% |
| NO: | Shaker5G_ | ACAGTTGTCCACTG | 1/0 asymmetric bulge at −11 | ADAR2 on target: |
| 219 | 256_ | CT | position (G-) | 92.00% |
| FIG. | gID_01981_ | | 1/0 asymmetric bulge at −6 | ADAR1 specificity: 1.6 |
| 218 | v0156 | | position (G-) | ADAR2 specificity: 1.9 |
| FIG. | | | 2/2 symmetric bulge at −1 | |
| 225 | | | position spanning 0 position | |
| | | | (GA-CG) | |
| | | | 1/1 C/A mismatch at +7 | |
| | | | position | |
| | | | 1/1 U/G wobble base pair at | |
| | | | +10 position | |
| SEQ | >ABCA4_ | CCCACCTCTCCAGG | 2/1 asymmetric bulge at −11 | ADAR1 on target: |
| ID | guide06_ | ACGAACTCGGACA | position (GC-A) | 85.34% |
| NO: | Shaker5G_ | ACAGATGTCCACTG | 1/0 asymmetric bulge at −6 | ADAR2 on target: |
| 220 | 256_ | CT | position (G-) | 81.67% |
| FIG. | gID_01981_ | | 2/2 symmetric bulge at −1 | ADAR1 specificity: |
| 218 | v0025 | | position spanning 0 position | 1.84 |
| FIG. | | | (GA-CG) | ADAR2 specificity: 1.8 |
| 226 | | | 1/1 C/A mismatch at +7 | |
| | | | position | |
| SEQ | >ABCA4_ | CCCACCTCTCCGGG | 1/1 G/U wobble base pair at | ADAR1 on target: |
| ID | guide06_ | GCGAACTCGGACA | −12 position | 73.27% |
| NO: | Shaker5G_ | ACAGTTGTCCACTG | 1/0 asymmetric bulge at −11 | ADAR2 on target: |
| 221 | 256_ | CT | position (G-) | 82.46% |
| FIG. | gID_01981_ | | 1/0 asymmetric bulge at −6 | ADAR1 specificity: |
| 218 | v0220 | | position (G-) | 1.64 |
| FIG. | | | 2/2 symmetric bulge at −1 | ADAR2 specificity: 1.8 |
| 227 | | | position spanning 0 position | |
| | | | (GA-CG) | |
| | | | 1/1 U/G wobble base pair at | |
| | | | +10 position | |
| SEQ | >ABCA4_ | CCCACCTCTCCAGG | 1/1 U/U mismatch at −3 | ADAR1 on target: |
| ID | guide01_ | GCGAATTTGGTCAC | position | 76.06% |
| NO: | CAPS1_128_ | ACAGCCTGTCCACT | 1/1 G/G mismatch at −1 | ADAR2 on target: |
| 222 | gID_ | GCT | position | 83.92% |
| FIG. | 00001_v0115 | | 1/1 G/U wobble base pair at | ADAR1 specificity: |
| 218 | | | +2 position | 1.68 |
| FIG. | | | | ADAR2 specificity: |
| 228 | | | | 1.42 |
| SEQ | >ABCA4_ | CCCACCTCTCCAGG | 1/1 G/G mismatch at −1 | ADAR1 on target: |
| ID | guide01_ | GCGAACTTGGACAC | position | 67.69% |
| NO: | CAPS1_128_ | ACAGCCTGTCCACT | | ADAR2 on target: |
| 223 | gID_ | GCT | | 81.74% |
| FIG. | 00001_v0081 | | | ADAR1 specificity: |
| 218 | | | | 1.63 |
| FIG. | | | | ADAR2 specificity: |
| 229 | | | | 1.51 |
| SEQ | >ABCA4_ | CCCACCGCTCCAGG | 1/1 U/U mismatch at −3 | ADAR1 on target: |
| ID | guide01_ | GCGAACTTGGTCAC | position | 38.81% |
| NO: | CAPS1_128_ | ACAGCCTGTCCACT | 1/1 G/G mismatch at −1 | ADAR2 on target: |
| 224 | gID_ | GCT | position | 83.53% |
| FIG. | 00001_v0019 | | 1/1 A/G mismatch at +15 | ADAR1 specificity: |
| 218 | | | position | 1.37 |
| FIG. | | | | ADAR2 specificity: |
| 230 | | | | 1.34 |
| SEQ | >ABCA4_ | CCCACCTCTCCGGG | 2/1 asymmetric bulge at −11 | ADAR1 on target: |
| ID | guide06_ | ACGAACTCGGACA | position (GG-A) | 76.99% |
| NO: | Shaker5G_ | ACAGATGTCCACTG | 1/0 asymmetric bulge at −6 | ADAR2 on target: |
| 225 | 256_ | CT | position (G-) | 86.79% |
| FIG. | gID_01981_ | | 2/2 symmetric bulge at −1 | ADAR1 specificity: |
| 218 | v0153 | | position spanning 0 position | 1.76 |
| FIG. | | | (GA-CG) | ADAR2 specificity: |
| 231 | | | 1/1 C/A mismatch at +7 | 1.86 |
| | | | position | |
| | | | 1/1 U/G wobble base pair at | |
| | | | +10 position | |
| SEQ | >ABCA4_ | CCCACCTCTCCAGG | 1/1 G/G mismatch at −11 | ADAR1 on target: |
| ID | guide05_ | ACGAACTCGGACA | position | 75.98% |
| NO: | Shaker5G_ | CACAGGCTGTCCAC | 2/2 symmetric bulge at −1 | ADAR2 on target: |
| 226 | 256_ | TGCT | position spanning 0 position | 81.74% |
| FIG. | gID_01585_ | | (GA-CG) | ADAR1 specificity: |
| 219 | v0027 | | 1/1 C/A mismatch at +7 | 1.74 |
| FIG. | | | position | ADAR2 specificity: |
| 232 | | | | 1.78 |
| SEQ | >ABCA4_ | CCCACCTCTTCAGG | 1/1 G/G mismatch at −1 | ADAR1 on target: |
| ID | guide08_ | GCGATCTTGGACAC | position | 71.82% |
| NO: | AJUBA_512_ | ACAGCCTGTCCACT | 1/1 U/U mismatch at +3 | ADAR2 on target: |
| 227 | gID_02773_ | GCT | position | 82.46% |
| FIG. | v0446 | | 1/1 G/U wobble base pair at | ADAR1 specificity: |
| 219 | | | +12 position | 1.66 |
| FIG. | | | | ADAR2 specificity: |
| 233 | | | | 1.52 |
| SEQ | >ABCA4_ | CCCACCTCTCCAGG | 2/1 asymmetric bulge at −11 | ADAR1 on target: |
| ID | guide06_ | ACGAACTCGGACA | position (GG-A) | 82.07% |
| NO: | kShaer5G_ | ACAGCTGTCCACTG | 1/0 asymmetric bulge at −6 | ADAR2 on target: |
| 228 | 256_ | CT | position (G-) | 85.49% |
| FIG. | gID_01981_ | | 2/2 symmetric bulge at −1 | ADAR1 specificity: |
| 219 | v0026 | | position spanning 0 position | 1.82 |
| FIG. | | | (GA-CG) | ADAR2 specificity: |
| 234 | | | 1/1 C/A mismatch at +7 | 1.84 |
| | | | position | |
| SEQ | >ABCA4_ | CCCACCTCTTCAGG | 1/1 G/G mismatch at −1 | ADAR1 on target: |
| ID | guide08_ | GCGAACTTGGACAC | position | 71.05% |
| NO: | AJUBA_512_ | ACAGCCTGTCCACT | 1/1 G/U wobble base pair at | ADAR2 on target: |
| 229 | gID_02773_ | GCT | +12 position | 81.51% |
| FIG. | v0414 | | | ADAR1 specificity: |
| 219 | | | | 1.67 |
| FIG. | | | | ADAR2 specificity: |
| 235 | | | | 01.51 |
| SEQ | >ABCA4_ | CCCACCGCTCCAGG | 1/1 G/U wobble base pair at | ADAR1 on target: |
| ID | guide01_ | GCGAACTTGGACAT | −6 position | 49.09% |
| NO: | CAPS1_128_ | ACAGCCTGTCCACT | 1/1 G/G mismatch at −1 | ADAR2 on target: |
| 230 | gID_00001_ | GCT | position | 87.42% |
| FIG. | 0v018 | | 1/1 A/G mismatch at +15 | ADAR1 specificity: |
| 219 | | | position | 1.46 |
| FIG. | | | | ADAR2 specificity: |
| 236 | | | | 1.37 |
| SEQ | >ABCA4_ | CCCACCTCTCCGGG | 2/1 asymmetric bulge at −11 | ADAR1 on target: |
| ID | guide06_ | ACGAACTCGGACA | position (GG-A) | 67.94% |
| NO: | Shaker5G_ | ACAGCTGTCCACTG | 1/0 asymmetric bulge at −6 | ADAR2 on target: |
| 231 | 256_ | CT | position (G-) | 88.82% |
| FIG. | gID_01981_ | | 2/2 symmetric bulge at −1 | ADAR1 specificity: |
| 219 | v0154 | | position spanning 0 position | 1.68 |
| FIG. | | | (GA-CG) | ADAR2 specificity: |
| 237 | | | 1/1 C/A mismatch at +7 | 1.87 |
| | | | position | |
| | | | 1/1 U/G wobble base pair at | |
| | | | +10 position | |
| SEQ | >ABCA4_ | CCCACCGCTCCAGG | 1/1 G/U wobble base pair at | ADAR1 on target: |
| ID | guide01_ | GCGAATTTGGTCAT | −6 position | 38.38% |
| NO: | CAPS1_128_ | ACAGCCTGTCCACT | 1/1 U/U mismatch at −3 | ADAR2 on target: |
| 232 | gID_ | GCT | position | 90.74% |
| FIG. | 00001_v0052 | | 1/1 G/G mismatch at −1 | ADAR1 specificity: |
| 219 | | | position | 1.36 |
| FIG. | | | 1/1 G/U wobble base pair at | ADAR2 specificity: |
| 238 | | | +2 position | 1.35 |
| | | | 1/1 A/G mismatch at +15 | |
| | | | position | |
| SEQ | >ABCA4_ | CCCACCGCTCCAGG | 1/1 G/U wobble base pair at | ADAR1 on target: |
| ID | guide01_ | GCGAATTTGGACAT | −6 position | 53.60% |
| NO: | CAPS1_128_ | ACAGCCTGTCCACT | 1/1 G/G mismatch at −1 | ADAR2 on target: |
| 233 | gID_ | GCT | position | 91.32% |
| FIG. | 00001_v0050 | | 1/1 G/U wobble base pair at | ADAR1 specificity: |
| 219 | | | +2 position | 1.52 |
| FIG. | | | 1/1 A/G mismatch at +15 | ADAR2 specificity: |
| 239 | | | position | 1.45 |
| SEQ | >ABCA4_ | CCGACCTCTTCAGG | 1/1 G/G mismatch at −1 | ADAR1 on target: |
| ID | guide08_ | GCGATCTTGGACAC | position | 61.32% |
| NO | AJUBA_512_ | ACAGCCTGTCCACT | 1/1 U/U mismatch at +3 | ADAR2 on target: |
| 234 | gID_02773_ | GCT | position | 83.41% |
| FIG. | v0190 | | 1/1 G/U wobble base pair at | ADAR1 specificity: |
| 220 | | | +12 position | 1.56 |
| FIG. | | | 1/1 G/G mismatch at +19 | ADAR2 specificity: |
| 240 | | | position | 1.54 |
| SEQ | >ABCA4_ | CCCACCTCTTCAGG | 1/1 C/A mismatch at −10 | ADAR1 on target: |
| ID | guide08_ | GCGATCTTGGACAC | position | 75.23% |
| NO: | AJUBA_512_ | ACAACCTGTCCACT | 1/1 G/G mismatch at −1 | ADAR2 on target: |
| 235 | gID_02773_ | GCT | position | 81.74% |
| FIG. | v0445 | | 1/1 U/U mismatch at +3 | ADAR1 specificity: |
| 220 | | | position | 1.74 |
| FIG. | | | 1/1 G/U wobble base pair at | ADAR2 specificity: |
| 241 | | | +12 position | 1.67 |
| SEQ | >ABCA4_ | CCCACCTCTCCAGG | 1/1 G/U wobble base pair at | ADAR1 on target: |
| ID | guide01_ | GCGAATTTGGTCAT | −6 position | 71.78% |
| NO: | CAPS1_128_ | ACAGCCTGTCCACT | 1/1 U/U mismatch at −3 | ADAR2 on target: |
| 236 | gID_00001_ | GCT | position | 87.83% |
| FIG. | v0116 | | 1/1 G/G mismatch at −1 | ADAR1 specificity: |
| 220 | | | position | 1.69 |
| FIG. | | | 1/1 G/U wobble base pair at | ADAR2 specificity: |
| 242 | | | +2 position | 1.31 |
| SEQ | >ABCA4_ | CCCACCTCTCCAGG | 1/1 G/U wobble base pair at | ADAR1 on target: |
| ID | guide06_ | ACGAACTCGGACA | −12 position | 75.77% |
| NO: | Shaker5G_ | ACAGTTGTCCACTG | 2/1 asymmetric bulge at −11 | ADAR2 on target: |
| 237 | 256_ | CT | position (GG-A) | 88.24% |
| FIG. | gID_01981_ | | 1/0 asymmetric bulge at −6 | ADAR1 specificity: |
| 220 | v0028 | | position (G-) | 1.64 |
| FIG. | | | 2/2 symmetric bulge at −1 | ADAR2 specificity: |
| 243 | | | position spanning 0 position | 1.86 |
| | | | (GA-CG) | |
| | | | 1/1 C/A mismatch at +7 | |
| | | | position | |
| SEQ | >ABCA4_ | CCGACCTCTCCAGG | 1/1 G/G mismatch at −1 | ADAR1 on target: |
| ID | ugide08_ | GCGATCTTGGACAC | position | 77.88% |
| NO: | AJUBA_512_ | ACAGCCTGTCCACT | 1/1 U/U mismatch at +3 | ADAR2 on target: |
| 238 | gID_02773_ | GCT | position | 80.23% |
| FIG. | v0062 | | 1/1 G/G mismatch at +19 | ADAR1 specificity: |
| 220 | | | position | 1.75 |
| FIG. | | | | ADAR2 specificity: |
| 244 | | | | 1.48 |
| SEQ | >ABCA4_ | CCGACCTCTTCAGG | 1/1 C/A mismatch at −10 | ADAR1 on target: |
| ID | guide08_ | GCGATCTTGGACAC | position | 67.61% |
| NO: | BAJUA_512_ | ACAACCTGTCCACT | 1/1 G/G mismatch at −1 | ADAR2 on target: |
| 239 | gID_02773_ | GCT | position | 80.01% |
| FIG. | v0189 | | 1/1 U/U mismatch at +3 | ADAR1 specificity: |
| 220 | | | position | 1.65 |
| FIG. | | | 1/1 G/U wobble base pair at | ADAR2 specificity: |
| 245 | | | +12 position | 1.67 |
| | | | 1/1 G/G mismatch at +19 | |
| | | | position | |
| SEQ | >ABCA4_ | CCCACCTCTCCAGG | 1/1 G/U wobble base pair at | ADAR1 on target: |
| ID | guide01_ | GCGAACTTGGACAT | −6 position | 78.67% |
| NO: | CAPS1_128_ | ACAGCCTGTCCACT | 1/1 G/G mismatch at −1 | ADAR2 on target: |
| 240 | gID_00001_ | GCT | position | 89.22% |
| FIG. | v0082 | | | ADAR1 specificity: |
| 220 | | | | 1.75 |
| FIG. | | | | ADAR2 specificity: |
| 246 | | | | 1.35 |
| SEQ | >ABCA4_ | CCGACCTCTTCAGG | 2/2 symmetric bulge at −1 | ADAR1 on target: |
| ID | guide08_ | GCGAACTCGGACA | position spanning 0 position | 56.22% |
| NO: | BAJUA _512_ | CACAGCCTGTCCAC | (GA-CG) | ADAR2 on target: |
| 241 | gID_02773_ | TGCT | 1/1 G/U wobble base pair at | 81.20% |
| FIG. | v0142 | | +12 position | ADAR1 specificity: |
| 220 | | | 1/1 G/G mismatch at +19 | 1.52 |
| FIG. | | | position | ADAR2 specificity: |
| 247 | | | | 1.59 |
| SEQ | >ABCA4_ | CCCACCTCTCCGGG | 1/1 G/G mismatch at −11 | ADAR1 on target: |
| ID | ugide05_ | ACGAACTCGGACA | position | 66.74% |
| NO: | Shaker5G_ | CACAGGCTGTCCAC | 2/2 symmetric bulge at −1 | ADAR2 on target: |
| 242 | 256_ | TGCT | position spanning 0 position | 82.99% |
| FIG. | gID_01585_ | | (GA-CG) | ADAR1 specificity: |
| 221 | v0155 | | 1/1 C/A mismatch at +7 | 1.65 |
| FIG. | | | position | ADAR2 specificity: |
| 248 | | | 1/1 U/G wobble base pair at | 1.77 |
| | | | +10 position | |
| SEQ | >ABCA4_ | CCCACCTCTCCGGG | 2/1 asymmetric bulge at −11 | ADAR1 on target: |
| ID | guide06_ | ACGAACTCGGACA | position (GG-G) | 79.65% |
| NO: | Shaker5G_ | ACAGGTGTCCACTG | 1/0 asymmetric bulge at −6 | ADAR2 on target: |
| 243 | 256_ | CT | position (G-) | 83.65% |
| FIG. | gID_01981_ | | 2/2 symmetric bulge at −1 | ADAR1 specificity: 1.8 |
| 221 | v0155 | | position spanning 0 position | ADAR2 specificity: |
| FIG. | | | (GA-CG) | 1.83 |
| 249 | | | 1/1 C/A mismatch at +7 | |
| | | | position | |
| | | | 1/1 U/G wobble base pair at | |
| | | | +10 position | |
| SEQ | >ABCA4_ | CCCACCTCTCCAGG | 1/1 G/G mismatch at −1 | ADAR1 on target: |
| ID | guide01 | GCGAATTTGGACAC | position | 83.97% |
| NO: | CAPS1_128_ | ACAGCCTGTCCACT | 1/1 G/U wobble base pair at | ADAR2 on target: |
| 244 | gID_ | GCT | +2 position | 81.88% |
| FIG. | 00001_v0113 | | | ADAR1 specificity: 1.8 |
| 221 | | | | ADAR2 specificity: |
| FIG. | | | | 1.53 |
| 250 | | | | |
| SEQ | >ABCA4_ | CCCACCGCTCCAGG | 1/1 G/U wobble base pair at | ADAR1 on target: |
| ID | guide02_ | GCGAACTTATCACA | −6 position | 23.40% |
| NO: | CAPS1_512_ | TACAGCCTGTCCAC | 2/3 asymmetric bulge at −1 | ADAR2 on target: |
| 245 | gID_ | TGCT | position spanning 0 position | 88.15% |
| FIG. | 00397_v0030 | | (CG-AUC) | ADAR1 specificity: |
| 221 | | | 1/1 A/G wobble base pair at | 1.23 |
| FIG. | | | +15 position | ADAR2 specificity: |
| 251 | | | | 1.49 |
| SEQ | >ABCA4_ | CCCACCTCTCCAGG | 1/1 G/U wobble base pair at | ADAR1 on target: |
| ID | guide01_ | GCGAACTTGGTCAT | −6 position | 74.79% |
| NO: | CAPS1 128_ | ACAGCCTGTCCACT | 1/1 U/U mismatch at −3 | ADAR2 on target: |
| 246 | gID00001_ | GCT | position | 86.23% |
| FIG. | v0084 | | 1/1 G/G mismatch at −1 | ADAR1 specificity: |
| 221 | | | position | 1.72 |
| FIG. | | | | ADAR2 specificity: |
| 252 | | | | 1.23 |
| SEQ | >ABCA4_ | CCCACCGCTCCAGG | 1/1 G/G mismatch at −1 | ADAR1 on target: |
| ID | guide01_ | GCGAATTTGGACAC | position | 53.83% |
| NO: | CAPS1_128_ | ACAGCCTGTCCACT | 1/1 G/U wobble base pair at | ADAR2 on target: |
| 247 | gID00001_ | GCT | +2 position | 86.56% |
| FIG. | v0049 | | 1/1 A/G wobble base pair at | ADAR1 specificity: |
| 221 | | | +15 position | 1.53 |
| FIG. | | | | ADAR2 specificity: |
| 253 | | | | 1.59 |
| SEQ | >ABCA4_ | CCCACCGCTCCAGG | 1/1 G/U wobble base pair at | ADAR1 on target: |
| ID | guide01_ | GCGAACTTGGTCAT | −6 position | 36.67% |
| NO: | CAPS1_128_ | ACAGCCTGTCCACT | 1/1 U/U mismatch at −3 | ADAR2 on target: |
| 248 | gID_ | GCT | position | 87.11% |
| FIG. | 00001_v0020 | | 1/1 G/G mismatch at −1 | ADAR1 specificity: |
| 221 | | | position | 1.34 |
| FIG. | | | 1/1 A/G wobble base pair at | ADAR2 specificity: |
| 254 | | | +15 position | 1.21 |
| SEQ | >ABCA4_ | CCCACCGCTCCAGG | 1/1 U/U mismatch at −3 | ADAR1 on target: |
| ID | guide01_ | GCGAATTTGGTCAC | position | 40.04% |
| NO: | CAPS1_128_ | ACAGCCTGTCCACT | 1/1 G/G mismatch at −1 | ADAR2 on target: |
| 249 | gID_ | GCT | position | 85.77% |
| FIG. | 00001_v0051 | | 1/1 G/U wobble base pair at | ADAR1 specificity: |
| 221 | | | +2 position | 1.38 |
| FIG. | | | 1/1 A/G wobble base pair at | ADAR2 specificity: |
| 255 | | | +15 position | 1.48 |

Select engineered guide RNAs discovered from the high throughput screen described above were further adapted (e.g., the main segment of the guide RNA that forms the structural features upon hybridization was trimmed and/or elongated to a ˜100mer guide RNA) for in trans editing of ABCA4. The elongated 100mer guide RNAs tested in cells were engineered such that the structural features in the guide target RNA scaffold would be positioned asymmetrically (towards the 5′ end of the target and the 3′ end of the guide RNA). In these cellular experiments, the target adenosine was positioned to be across from around the 80^{th} nucleotide of the guide RNA.

HEK293 cells naturally expressing ADAR1 were transfected with a piggyBac vector carrying the ABCA4 minigene and ADAR2. Engineered guide RNAs were administered to cells and RNA editing was quantified 48 hours post transfection. As shown in FIG. 46A, guides in which the A/C (target/guide) mismatch was designed to occur at position 80 from the 5′ end of the engineered guide RNA facilitated higher percent RNA editing of the ABCA4 5882 G->A mutation.

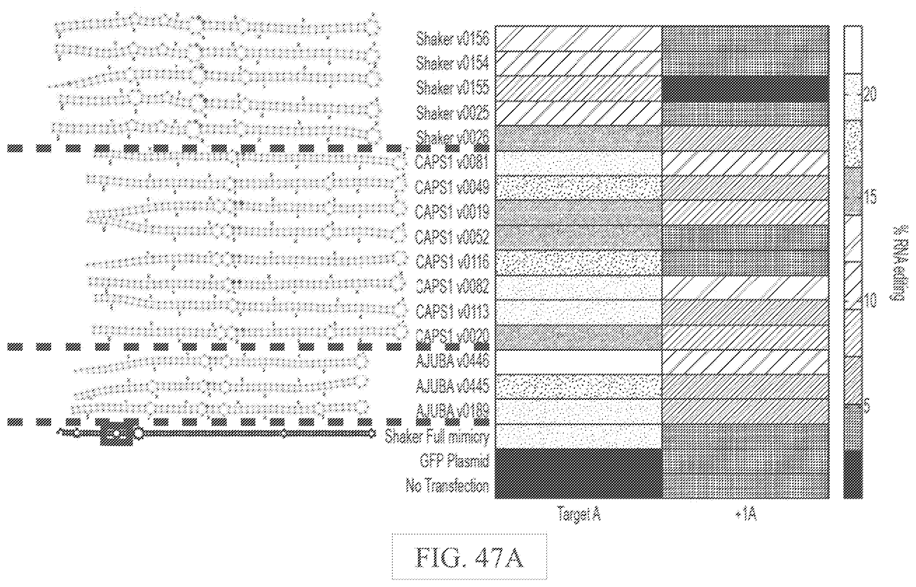

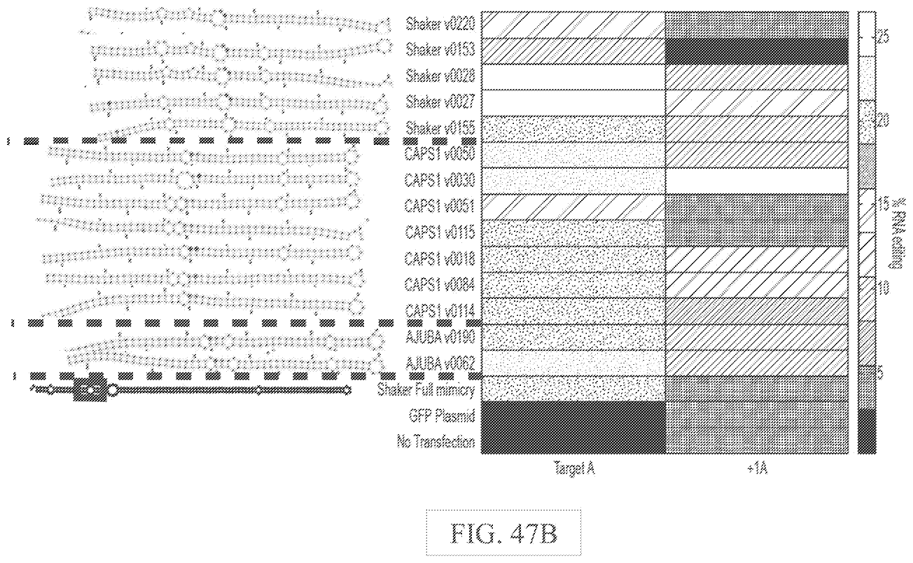

FIG. 47A and FIG. 47B show heatmaps illustrating percent RNA editing of the ABCA4 G5882A missense mutation facilitated by engineered polynucleotides encoding U1 promoter driven guide RNAs with an SmOPT sequence and a U7 hairpin sequence, where RNA editing was facilitated by ADAR1 and ADAR2. For these FIG. 47A and FIG. 47B, HEK293 cells naturally expressing ADAR1 were transfected with a piggyBac vector carrying the ABCA4 minigene and ADAR2. Engineered guide RNAs were administered to cells and RNA editing was quantified 48 hours post transfection. Heatmaps show the target A to be edited and an off-target A immediately 3′ of the target A to be edited. Structural features formed upon hybridization of the engineered guide RNA to the target ABCA4 RNA are shown at the left of the heatmaps in FIG. 47A and FIG. 47B. A summary of each engineered guide RNA is described below in TABLE 19.

Polynucleotide sequences encoding engineered guide RNAs targeting ABCA4 are provided below in TABLE 19. The column in TABLE 19 titled “Structural Features” describes structural features in the double stranded RNA substrate formed upon hybridization of the gRNA to the target RNA. The italicized sequence is the sequence of the 100mer engineered guide RNA.

TABLE 19
Guide RNA Sequences against ABCA4 for In Trans-Editing Experiments

| SEQ ID NO | | | Structural Features | % RNA |
|---|---|---|---|---|
| FIG | Name | Sequence | (target/guide) | Editing |
| SEQ ID NO: 250 | Shaker v0153 | TGCCGGCACCATTCACTCC | 1 C/A mismatch | 1 |
| FIG. 46A | U1 SmOPT | CAGGAGGCCAAAGCACTCT | 2/2 symmetrical bulge | |
| | 0.100.51 | CCGGGACGAACTCGGACA | 1/0 asymmetrical bulge | |
| | | ACAGATGTCCACTGCTGGG | 2/1 asymmetrical bulge | |
| | | CTGGAGGTGCCTGGATAAA | | |
| | | TCTTGGGgtgg | | |
| | | AATTTTTGGAG | | |
| | | CAGGTTTTCTGACTTCGG | | |
| | | TCGGAAAACCCCT | | |
| SEQ ID NO: 251 | Shaker v0153 | CCCAGTGAGCATCTTGAAT | 1 C/A mismatch | 8 |
| FIG. 46A | U1 SmOPT | GTGGTTGTTTTGCCGGCAC | 2/2 symmetrical bulge | |
| | 0.100.80 | CATTCACTCCCAGGAGGCC | 1/0 asymmetrical bulge | |
| | | AAAGCACTCTCCGGGACGA | 2/1 asymmetrical bulge | |
| | | ACTCGGACAACAGATGTCC | | |
| | | ACTGCgtgg | | |
| | | AATTTTTGGAG | | |
| | | CAGGTTTTCTGACTTCGG | | |
| | | TCGGAAAACCCCT | | |
| SEQ ID NO: 252 | Shaker v0155 | TGCCGGCACCATTCACTCC | 1 C/A mismatch | 7 |
| FIG. 46A | U1 SmOPT | CAGGAGGCCAAAGCACTCT | 2/2 symmetrical bulge | |
| | 0.100.51 | CCGGGACGAACTCGGACA | 1 G/G mismatch | |
| | | CACAGGCTGTCCACTGCTG | | |
| | | GGCTGGAGGTGCCTGGAT | | |
| | | AAATCTTgtgg | | |
| | | AATTTTTGGAG | | |
| | | CAGGTTTTCTGACTTCGG | | |
| | | TCGGAAAACCCCT | | |
| SEQ ID NO: 253 | Shaker v0155 | CCCAGTGAGCATCTTGAAT | 1 C/A mismatch | 19 |
| FIG. 46A | U1 SmOPT | GTGGTTGTTTTGCCGGCAC | 2/2 symmetrical bulge | |
| FIG. 47A | 0.100.80 | CATTCACTCCCAGGAGGCC | 1 G/G mismatch | |
| FIG. 47B | | AAAGCACTCTCCGGGACGA | | |
| | | ACTCGGACACACAGGCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 254 | AJUBA v0414 | TGCCGGCACCATTCACTCC | 1 G/G mismatch | 0.33 |
| FIG. 46A | U1 SmOPT | CAGGAGGCCAAAGCACTCT | | |
| | 0.100.49 | TCAGGGCGAACTTGGACAC | | |
| | | ACAGCCTGTCCACTGCTGG | | |
| | | GCTGGAGGTGCCTGGATA | | |
| | | AATCTTgtgg | | |
| | | AATTTTTGGAG | | |
| | | CAGGTTTTCTGACTTCGG | | |
| | | TCGGAAAACCCCT | | |
| SEQ ID NO: 255 | AJUBA v0414 | CCCAGTGAGCATCTTGAAT | 1 G/G mismatch | 21 |
| FIG. 46A | U1 SmOPT | GTGGTTGTTTTGCCGGCAC | | |
| | 0.100.78 | CATTCACTCCCAGGAGGCC | | |
| | | AAAGCACTCTTCAGGGCGA | | |
| | | ACTTGGACACACAGCCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 256 | AJUBA v0190 | TGCCGGCACCATTCACTCC | 1 G/G mismatch | 3 |
| FIG. 46A | U1 SmOPT | CAGGAGGCCAAAGCACTCT | 1 U/U mismatch | |
| | 0.100.49 | TCAGGGCGATCTTGGACAC | 1 G/G mismatch | |
| | | ACAGCCTGTCCACTGCTGG | | |
| | | GCTGGAGGTGCCTGGATA | | |
| | | AATCTTgtgg | | |
| | | AATTTTTGGAG | | |
| | | CAGGTTTTCTGACTTCGG | | |
| | | TCGGAAAACCCCT | | |
| SEQ ID NO: 257 | AJUBA v0190 | CCCAGTGAGCATCTTGAAT | 1 G/G mismatch | 21 |
| FIG. 46A | U1 SmOPT | GTGGTTGTTTTGCCGGCAC | 1 U/U mismatch | |
| FIG. 47B | 0.100.78 | CATTCACTCCCAGGAGGCC | 1 G/G mismatch | |
| | | AAAGCACTCTTCAGGGCGA | | |
| | | TCTTGGACACACAGCCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 258 | CAPS1 v0018 | TGCCGGCACCATTCACTCC | 1 G/A mismatch | 5 |
| FIG. 46A | U1 SmOPT | CAGGAGGCCAAAGCACGC | 1 G/G mismatch | |
| | 0.100.49 | TCCAGGGCGAACTTGGACA | | |
| | | TACAGCCTGTCCACTGCTG | | |
| | | GGCTGGAGGTGCCTGGAT | | |
| | | AAATCTTgtgg | | |
| | | AATTTTTGGAG | | |
| | | CAGGTTTTCTGACTTCGG | | |
| | | TCGGAAAACCCCT | | |
| SEQ ID NO: 259 | CAPS1 v0018 | CCCAGTGAGCATCTTGAAT | 1 G/A mismatch | 15 |
| FIG. 46A | U1 SmOPT | GTGGTTGTTTTGCCGGCAC | 1 G/G mismatch | |
| FIG. 47B | 0.100.78 | CATTCACTCCCAGGAGGCC | | |
| | | AAAGCACGCTCCAGGGCG | | |
| | | AACTTGGACATACAGCCTG | | |
| | | TCCACTgtgg | | |
| | | AATTTTTGGAG | | |
| | | CAGGTTTTCTGACTTCGG | | |
| | | TCGGAAAACCCCT | | |
| SEQ ID NO: 260 | CAPS1 v0084 | TGCCGGCACCATTCACTCC | 1 G/G mismatch | 0.33 |
| FIG. 46A | U1 SmOPT | CAGGAGGCCAAAGCACTCT | 1 U/U mismatch | |
| | 0.100.49 | CCAGGGCGAACTTGGTCAT | | |
| | | ACAGCCTGTCCACTGCTGG | | |
| | | GCTGGAGGTGCCTGGATA | | |
| | | AATCTTgtgg | | |
| | | AATTTTTGGAG | | |
| | | CAGGTTTTCTGACTTCGG | | |
| | | TCGGAAAACCCCT | | |
| SEQ ID NO: 261 | CAPS1 v0084 | CCCAGTGAGCATCTTGAAT | 1 G/G mismatch | 11 |
| FIG. 46A | Ul SmOPT | GTGGTTGTTTTGCCGGCAC | 1 U/U mismatch | |
| FIG. 47B | 0.100.78 | CATTCACTCCCAGGAGGCC | | |
| | | AAAGCACTCTCCAGGGCGA | | |
| | | ACTTGGTCATACAGCCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 262 | Shaker v0156 | CCCAGTGAGCATCTTGAAT | 1 C/A mismatch | 12 |
| FIG. 47A | U1 SmOPT | GTGGTTGTTTTGCCGGCAC | 2/2 symmetrical bulge | |
| | | CATTCACTCCCAGGAGGCC | 1/0 asymmetrical bulge | |
| | | AAAGCACTCTCCGGGACGA | 1/0 asymmetrical bulge | |
| | | ACTCGGACAACAGTTGTCC | | |
| | | ACTGCgtgg | | |
| | | AATTTTTGGAG | | |
| | | CAGGTTTTCTGACTTCGG | | |
| | | TCGGAAAACCCCT | | |
| SEQ ID NO: 263 | Shaker v0154 | CCCAGTGAGCATCTTGAAT | 1 C/A mismatch | 11 |
| FIG. 47A | U1 SmOPT | GTGGTTGTTTTGCCGGCAC | 2/2 symmetrical bulge | |
| | | CATTCACTCCCAGGAGGCC | 1/0 asymmetrical bulge | |
| | | AAAGCACTCTCCGGGACGA | 1/0 asymmetrical bulge | |
| | | ACTCGGACAACAGCTGTCC | | |
| | | ACTGCgtgg | | |
| | | AATTTTTGGAG | | |
| | | CAGGTTTTCTGACTTCGG | | |
| | | TCGGAAAACCCCT | | |
| SEQ ID NO: 265 | Shaker v0025 | CCCAGTGAGCATCTTGAAT | 1 C/A mismatch | 10 |
| FIG. 47A | U1 SmOPT | GTGGTTGTTTTGCCGGCAC | 2/2 symmetrical bulge | |
| | | CATTCACTCCCAGGAGGCC | 1/0 asymmetrical bulge | |
| | | AAAGCACTCTCCAGGACGA | 2/1 asymmetrical bulge | |
| | | ACTCGGACAACAGATGTCC | | |
| | | ACTGCgtgg | | |
| | | AATTTTTGGAG | | |
| | | CAGGTTTTCTGACTTCGG | | |
| | | TCGGAAAACCCCT | | |
| SEQ ID NO: 266 | Shaker v0026 | CCCAGTGAGCATCTTGAAT | 1 C/A mismatch | 15 |
| FIG. 47A | U1 SmOPT | GTGGTTGTTTTGCCGGCAC | 2/2 symmetrical bulge | |
| | | CATTCACTCCCAGGAGGCC | 1/0 asymmetrical bulge | |
| | | AAAGCACTCTCCAGGACGA | 1/0 asymmetrical bulge | |
| | | ACTCGGACAACAGCTGTCC | | |
| | | ACTGCgtgg | | |
| | | AATTTTTGGAG | | |
| | | CAGGTTTTCTGACTTCGG | | |
| | | TCGGAAAACCCCT | | |
| SEQ ID NO: 267 | CAPS1 v0081 | CCCAGTGAGCATCTTGAAT | 1 G/G mismatch | 20 |
| FIG. 47A | U1 SmOPT | GTGGTTGTTTTGCCGGCAC | | |
| | | CATTCACTCCCAGGAGGCC | | |
| | | AAAGCACTCTCCAGGGCGA | | |
| | | ACTTGGACACACAGCCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 268 | CAPS1 v0049 | CCCAGTGAGCATCTTGAAT | 1 A/G mismatch | 18 |
| FIG. 47A | U1 SmOPT | GTGGTTGTTTTGCCGGCAC | 1 G/G mismatch | |
| | | CATTCACTCCCAGGAGGCC | | |
| | | AAAGCACGCTCCAGGGCG | | |
| | | AATTTGGACACACAGCCTG | | |
| | | TCCACTgtgg | | |
| | | AATTTTTGGAG | | |
| | | CAGGTTTTCTGACTTCGG | | |
| | | TCGGAAAACCCCT | | |
| SEQ ID NO: 269 | CAPS1 v0019 | CCCAGTGAGCATCTTGAAT | 1 A/G mismatch | 15 |
| FIG. 47A | U1 SmOPT | GTGGTTGTTTTGCCGGCAC | 1 G/G mismatch | |
| | | CATTCACTCCCAGGAGGCC | 1 U/U mismatch | |
| | | AAAGCACGCTCCAGGGCG | | |
| | | AACTTGGTCACACAGCCTG | | |
| | | TCCACTgtgg | | |
| | | AATTTTTGGAG | | |
| | | CAGGTTTTCTGACTTCGG | | |
| | | TCGGAAAACCCCT | | |
| SEQ ID NO: 270 | CAPS1 v0052 | CCCAGTGAGCATCTTGAAT | 1 G/A mismatch | 14 |
| FIG. 47A | U1 SmOPT | GTGGTTGTTTTGCCGGCAC | 1 G/G mismatch | |
| | | CATTCACTCCCAGGAGGCC | 1 U/U mismatch | |
| | | AAAGCACGCTCCAGGGCG | | |
| | | AATTTGGTCATACAGCCTG | | |
| | | TCCACTgtgg | | |
| | | AATTTTTGGAG | | |
| | | CAGGTTTTCTGACTTCGG | | |
| | | TCGGAAAACCCCT | | |
| SEQ ID NO: 271 | CAPS1 v0116 | CCCAGTGAGCATCTTGAAT | 1 G/G mismatch | 16 |
| FIG. 47A | U1 SmOPT | GTGGTTGTTTTGCCGGCAC | 1 U/U mismatch | |
| | | CATTCACTCCCAGGAGGCC | | |
| | | AAAGCACTCTCCAGGGCGA | | |
| | | ATTTGGTCATACAGCCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 272 | CAPS1 v0082 | CCCAGTGAGCATCTTGAAT | 1 G/G mismatch | 20 |
| FIG. 47A | U1 SmOPT | GTGGTTGTTTTGCCGGCAC | | |
| | | CATTCACTCCCAGGAGGCC | | |
| | | AAAGCACTCTCCAGGGCGA | | |
| | | ACTTGGACATACAGCCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 273 | CAPS1 v0113 | CCCAGTGAGCATCTTGAAT | 1 G/G mismatch | 20 |
| FIG. 47A | U1 SmOPT | GTGGTTGTTTTGCCGGCAC | | |
| | | CATTCACTCCCAGGAGGCC | | |
| | | AAAGCACTCTCCAGGGCGA | | |
| | | ATTTGGACACACAGCCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 274 | CAPS1 v0020 | CCCAGTGAGCATCTTGAAT | 1 A/G mismatch | 14 |
| FIG. 47A | U1 SmOPT | GTGGTTGTTTTGCCGGCAC | 1 G/G mismatch | |
| | | CATTCACTCCCAGGAGGCC | 1 U/U mismatch | |
| | | AAAGCACGCTCCAGGGCG | | |
| | | AACTTGGTCATACAGCCTG | | |
| | | TCCACTgtgg | | |
| | | AATTTTTGGAG | | |
| | | CAGGTTTTCTGACTTCGG | | |
| | | TCGGAAAACCCCT | | |
| SEQ ID NO: 275 | AJUBA v0446 | CCCACCTCTTCAGGGCGAT | 1 U/U mismatch | 23 |
| FIG. 47A | U1 SmOPT | CTTGGACACACAGCCTGTC | 1 G/G mismatch | |
| | | CACTGCTgtgg | | |
| | | AATTTTTGGAG | | |
| | | CAGGTTTTCTGACTTCGG | | |
| | | TCGGAAAACCCCT | | |
| SEQ ID NO: 276 | AJUBA v0445 | CCCACCTCTTCAGGGCGAT | 1 U/U mismatch | 18 |
| FIG. 47A | U1 SmOPT | CTTGGACACACAACCTGTC | 1 G/G mismatch | |
| | | CACTGCTgtgg | 1 C/A mismatch | |
| | | AATTTTTGGAG | | |
| | | CAGGTTTTCTGACTTCGG | | |
| | | TCGGAAAACCCCT | | |
| SEQ ID NO: 277 | AJUBA v0189 | CCGACCTCTTCAGGGCGAT | 1 G/G mismatch | 20 |
| FIG. 47A | U1 SmOPT | CTTGGACACACAACCTGTC | 1 U/U mismatch | |
| | | CACTGCTgtgg | 1 G/G mismatch | |
| | | AATTTTTGGAG | 1 C/A mismatch | |
| | | CAGGTTTTCTGACTTCGG | | |
| | | TCGGAAAACCCCT | | |
| SEQ ID NO: 278 | Shaker v0220 | CCCAGTGAGCATCTTGAAT | 2/2 symmetrical bulge | 13 |
| FIG. 47B | U1 SmOPT | GTGGTTGTTTTGCCGGCAC | 1/0 asymmetrical bulge | |
| | | CATTCACTCCCAGGAGGCC | 1/0 asymmetrical bulge | |
| | | AAAGCACTCTCCGGGGCG | | |
| | | AACTCGGACAACAGTTGTC | | |
| | | CACTGCgtgg | | |
| | | AATTTTTGGAG | | |
| | | CAGGTTTTCTGACTTCGG | | |
| | | TCGGAAAACCCCT | | |
| SEQ ID NO: 279 | Shaker v0153 | CCCAGTGAGCATCTTGAAT | 1 C/A mismatch | 8 |
| FIG. 47B | U1 SmOPT | GTGGTTGTTTTGCCGGCAC | 2/2 symmetrical bulge | |
| | | CATTCACTCCCAGGAGGCC | 1/0 asymmetrical bulge | |
| | | AAAGCACTCTCCGGGACGA | 2/1 asymmetrical bulge | |
| | | ACTCGGACAACAGATGTCC | | |
| | | ACTGCgtgg | | |
| | | AATTTTTGGAG | | |
| | | CAGGTTTTCTGACTTCGG | | |
| | | TCGGAAAACCCCT | | |
| SEQ ID NO: 280 | Shaker v0028 | CCCAGTGAGCATCTTGAAT | 1 C/A mismatch | 24 |
| FIG. 47B | U1 SmOPT | GTGGTTGTTTTGCCGGCAC | 2/2 symmetrical bulge | |
| | | CATTCACTCCCAGGAGGCC | 1/0 asymmetrical bulge | |
| | | AAAGCACTCTCCAGGACGA | 1/0 asymmetrical bulge | |
| | | ACTCGGACAACAGTTGTCC | | |
| | | ACTGCgtgg | | |
| | | AATTTTTGGAG | | |
| | | CAGGTTTTCTGACTTCGG | | |
| | | TCGGAAAACCCCT | | |
| SEQ ID NO: 281 | Shaker v0027 | CCCAGTGAGCATCTTGAAT | 1 C/A mismatch | 27 |
| FIG. 47B | U1 SmOPT | GTGGTTGTTTTGCCGGCAC | 2/2 symmetrical bulge | |
| | | CATTCACTCCCAGGAGGCC | 1 G/G mismatch | |
| | | AAAGCACTCTCCAGGACGA | | |
| | | ACTCGGACACACAGGCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 283 | CAPS1 v0050 | CCCAGTGAGCATCTTGAAT | 1 G/A mismatch | 22 |
| FIG. 47B | U1 SmOPT | GTGGTTGTTTTGCCGGCAC | 1 G/G mismatch | |
| | | CATTCACTCCCAGGAGGCC | | |
| | | AAAGCACGCTCCAGGGCG | | |
| | | AATTTGGACATACAGCCTG | | |
| | | TCCACTgtgg | | |
| | | AATTTTTGGAG | | |
| | | CAGGTTTTCTGACTTCGG | | |
| | | TCGGAAAACCCCT | | |
| SEQ ID NO: 284 | CAPS1 v0030 | CCCAGTGAGCATCTTGAAT | 1 A/G mismatch | 22 |
| FIG. 47B | U1 SmOPT | GTGGTTGTTTTGCCGGCAC | 2/2 symmetrical bulge | |
| | | CATTCACTCCCAGGAGGCC | | |
| | | AAAGCACGCTCCAGGGCG | | |
| | | AACTTATCACATACAGCCT | | |
| | | GTCCACgtgg | | |
| | | AATTTTTGGAG | | |
| | | CAGGTTTTCTGACTTCGG | | |
| | | TCGGAAAACCCCT | | |
| SEQ ID NO: 285 | CAPS1 v0051 | CCCAGTGAGCATCTTGAAT | 1 A/G mismatch | 15 |
| FIG. 47B | U1 SmOPT | GTGGTTGTTTTGCCGGCAC | 1 G/G mismatch | |
| | | CATTCACTCCCAGGAGGCC | 1 U/U mismatch | |
| | | AAAGCACGCTCCAGGGCG | | |
| | | AATTTGGTCACACAGCCTG | | |
| | | TCCACTgtgg | | |
| | | AATTTTTGGAG | | |
| | | CAGGTTTTCTGACTTCGG | | |
| | | TCGGAAAACCCCT | | |
| SEQ ID NO: 286 | CAPS1 v0115 | CCCAGTGAGCATCTTGAAT | 1 G/G mismatch | 20 |
| FIG. 47B | U1 SmOPT | GTGGTTGTTTTGCCGGCAC | 1 U/U mismatch | |
| | | CATTCACTCCCAGGAGGCC | | |
| | | AAAGCACTCTCCAGGGCGA | | |
| | | ATTTGGTCACACAGCCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 289 | CAPS1 v0114 | CCCAGTGAGCATCTTGAAT | 1 G/G mismatch | 21 |
| FIG. 47B | U1 SmOPT | GTGGTTGTTTTGCCGGCAC | | |
| | | CATTCACTCCCAGGAGGCC | | |
| | | AAAGCACTCTCCAGGGCGA | | |
| | | ATTTGGACATACAGCCTGT | | |
| | | CCACTgtgg | | |
| | | AATTTTTGGAG | | |
| | | CAGGTTTTCTGACTTCGG | | |
| | | TCGGAAAACCCCT | | |
| SEQ ID NO: 291 | AJUBA v0062 | CCCAGTGAGCATCTTGAAT | 1 G/G mismatch | 23 |
| FIG. 47B | U1 SmOPT | GTGGTTGTTTTGCCGGCAC | 1 U/U mismatch | |
| | | CATTCACTCCCAGGAGGCC | 1 G/G mismatch | |
| | | AAAGCACTCTCCAGGGCGA | | |
| | | TCTTGGACACACAGCCTGT | | |
| | | CCACTgtgg | | |
| | | AATTTTTGGAG | | |
| | | CAGGTTTTCTGACTTCGG | | |
| | | TCGGAAAACCCCT | | |

### Example 11

### RNA Editing in Cells by LRRK2-Targeting Engineered Guide RNAs

This example describes in cell ADAR-mediated RNA editing of LRRK2, facilitated by engineered guide RNA of the present disclosure. Select engineered guide RNAs discovered from a high throughput screen described in EXAMPLE 9 were further adapted (e.g., the main segment of the guide RNA that forms the structural features upon hybridization was trimmed and/or elongated to a ˜100mer guide RNA) for in trans editing of LRRK2 in cells. Two guide RNA designs targeting the LRRK2 G2019S mutation were tested, both with a SmOPT sequence and a U7 hairpin (SEQ ID NO: 344 and SEQ ID NO: 345). The first engineered guide RNA (“V0118 0.100.50”) contained 100 nucleotides with the target A in LRRK2 to be edited across from the nucleotide at position 50 in the engineered guide RNA. The second engineered guide RNA (“V0118 0.100.80”) contained 100 nucleotides with the target A in LRRK2 to be edited across from the nucleotide at position 80 in the guide. The elongated 100mer guide RNAs tested in cells were engineered such that the structural features in the guide target RNA scaffold would be positioned symmetrically (towards the middle of the guide-target RNA scaffold; v0118 0.100.50) or asymmetrically (towards the 5′ end of the target and the 3′ end of the guide RNA; v0118 0.100.80). Both engineered guide RNAs further formed a 6/6 symmetrical internal loop and 2 other mismatches (an A/G mismatch and a C/U mismatch) apart from the A/C mismatch at the target adenosine. A summary of each engineered guide RNA is described below in TABLE 20. The column in TABLE 20 titled “Structural Features” describes structural features in the double stranded RNA substrate formed upon hybridization of the gRNA to the target RNA.

TABLE 20
Anti-LRRK2 Engineered Guide RNAs

| | | | | % RNA |
|---|---|---|---|---|
| | | | | Editing |
| | | | | [ADAR1, |
| | | Engineered Guide RNA | | ADAR1 + |
| SEQ ID NO | ID | Sequence | Structural Features | ADAR2] |
| SEQ ID NO: | V0118 | GTGCCCTCTGATGTTTTTATC | 6/6 symmetrical internal loop | [8, 28] |
| 344 | 0.100.50 | CCCATTCTACAGCATTACGG | 3 mismatches (A/C, A/G, C/U) | |
| | | AGCAGTGCCGTAGTGTCGTT | | |
| | | TCTTTGCAATGATGGCAGCA | | |
| | | TTGGGATACAGTGTGAAAAgt | | |
| | | ggAATTTTTGGAG | | |
| | | CAGGTTTTCTGACTTCGGTC | | |
| | | GGAAAACCCCT | | |
| SEQ ID NO: | V0118 | CTGGCAACTTCAGGTGCACG | 6/6 symmetrical internal loop | [19, 58] |
| 345 | 0.100.80 | AAACCCTGGTGTGCCCTCTG | 3 mismatches (A/C, A/G, C/U) | |
| | | ATGTTTTTATCCCCATTCTAC | | |
| | | AGCATTACGGAGCAGTGCCG | | |
| | | TAGTGTCGTTTCTTTGCAAgtg | | |
| | | gAATTTTTGGAG | | |

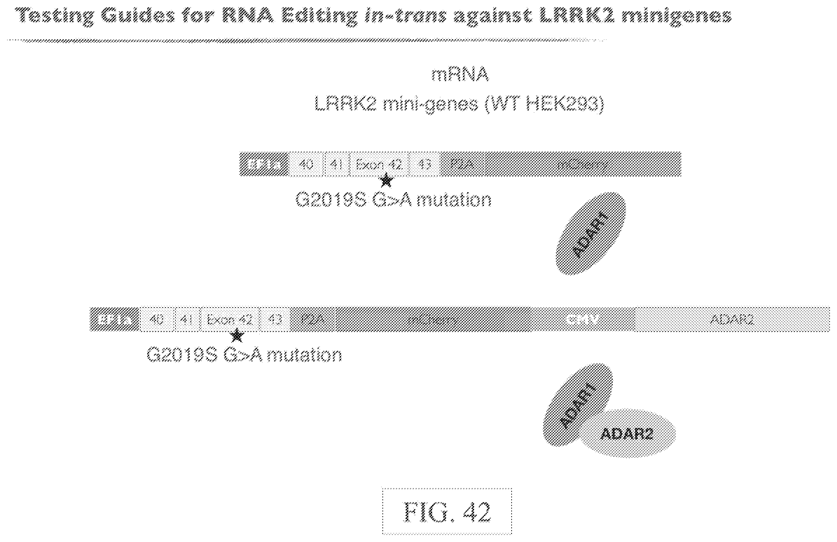

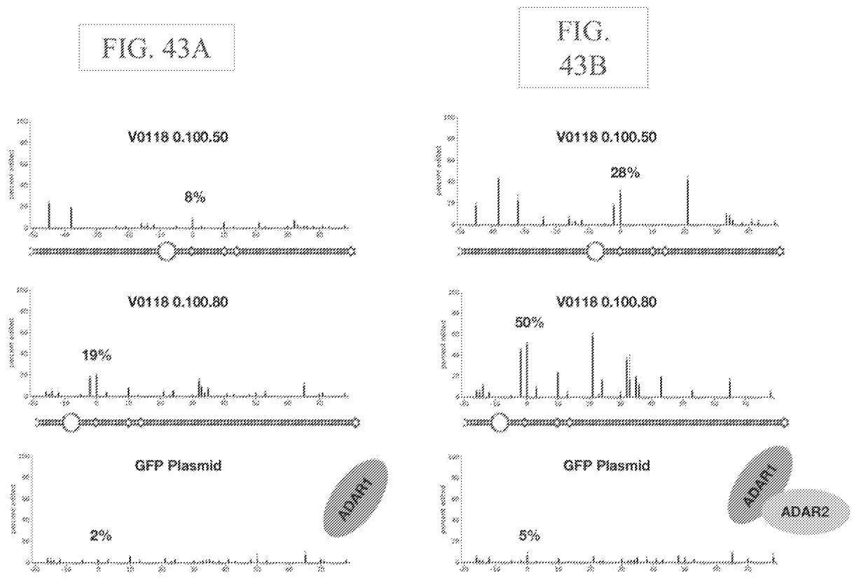

Engineered guide RNAs were tested for their ability to facilitate ADAR-mediated RNA editing of the G2019S LRRK2 mutation in WT HEK293 cells transfected with a piggyBac vector carrying a LRRK2 minigene. WT HEK293 cells naturally express ADAR1. In experiments in which RNA editing mediated via ADAR1 and ADAR2 was tested, ADAR2 was overexpressed in cells via the same piggyBac vector carrying the LRRK2 minigene. Schematics of the piggyBac constructs are shown in FIG. 42. Experiments were conducted in the presence of ADAR1 only (FIG. 43A) or ADAR1 and ADAR2 (FIG. 43B). A GFP plasmid was used as a control. FIG. 43A and FIG. 43B show that engineered guide RNAs containing a SmOPT sequence and a U7 hairpin sequence facilitated an on-target editing efficiency of 8% in the presence of ADAR1 only and 28% in the presence of ADAR1 and ADAR2. FIG. 43A and FIG. 43B show that guide RNAs containing a SmOPT sequence and a U7 hairpin sequence facilitated an on-target editing efficiency of 19% in the presence of ADAR1 only and 58% in the presence of ADAR1 and ADAR2. Further experimentation demonstrated that the first engineered guide RNA (“V0118 0.100.50”) had a Gibbs free energy (delta G) of −161.98 kcal/mol and the second engineered guide RNA (“V0118 0.100.80”) had a delta G of −169.44 kcal/mol. The structures of both engineered guide RNAs are shown beneath the graphs in FIG. 43A-FIG. 43B. As seen in the structural diagrams, the second engineered guide RNA (“V0118 0.100.50”) formed a longer continuous stretch of duplex RNA with the target RNA. These figures show that asymmetric positioning of the A/C mismatch (e.g., 0.100.80 designs) was generally preferable in gRNA designs as opposed to centering of the A/C mismatch (e.g., 0.100.50 designs).

### Example 12

### Engineered Guide RNAs Against SNCA

This example describes constructs of the present disclosure encoding engineered guide RNAs designed to target a start site, or translation initiation site (TIS) (also referred to as translation start site (TSS)) in the SNCA gene. Engineered guide constructs were designed to target SNCA TIS adenosine at nucleotide position 26 of Exon 2 (corresponding to nucleotide position 264 of most SNCA variants, including Exon 1 and Exon 2).

HEK293 cells were transfected with a plasmid encoding a guide RNA of interest and RNA editing was assessed 48 hours post-transfection. RNA editing was assessed for ADAR1 only, which is naturally expressed by HEK293 cells, and ADAR1 and ADAR2. In the latter experiment, HEK293 cells were co-transfected with a piggybac vector encoding ADAR2. Levels of RNA editing were quantified by Sanger sequencing and analyzed using a sequencing analysis script (EditADAR).

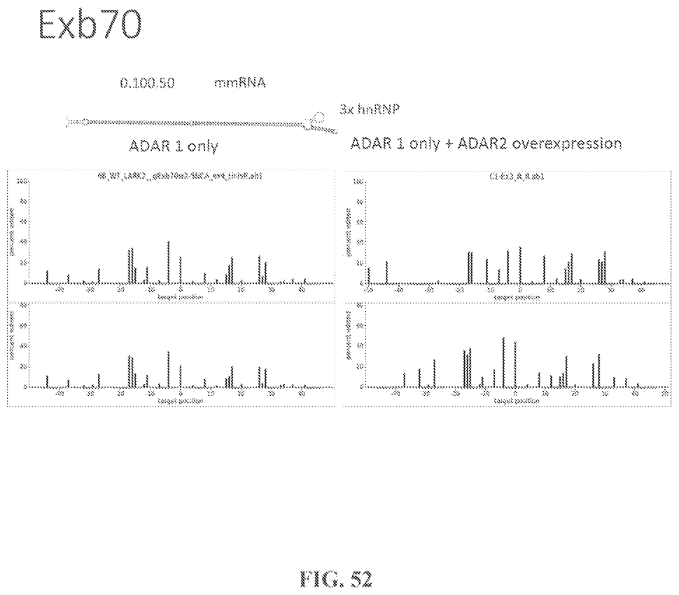

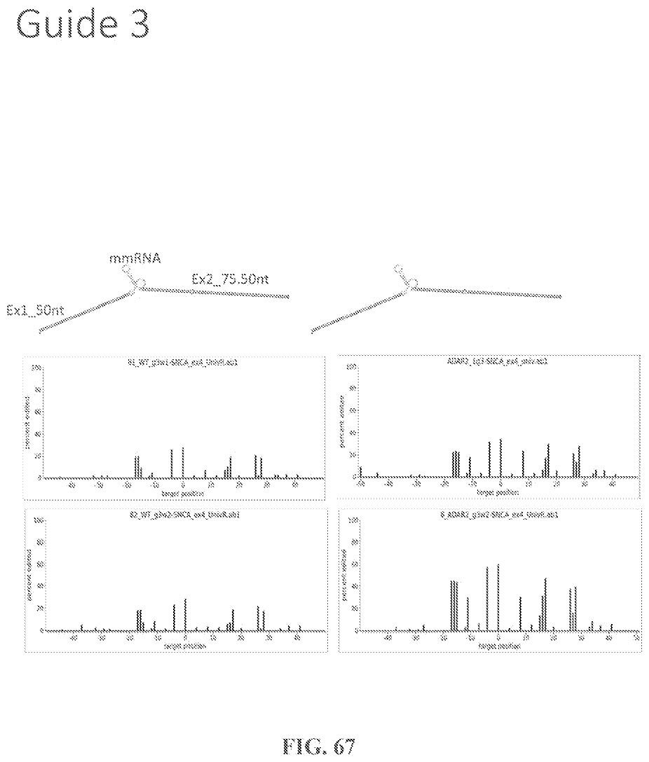

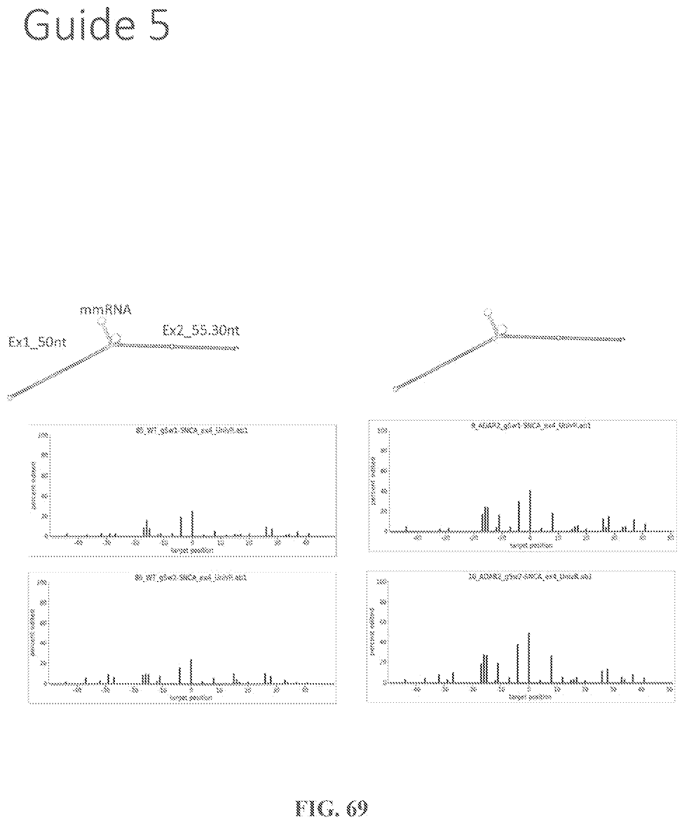

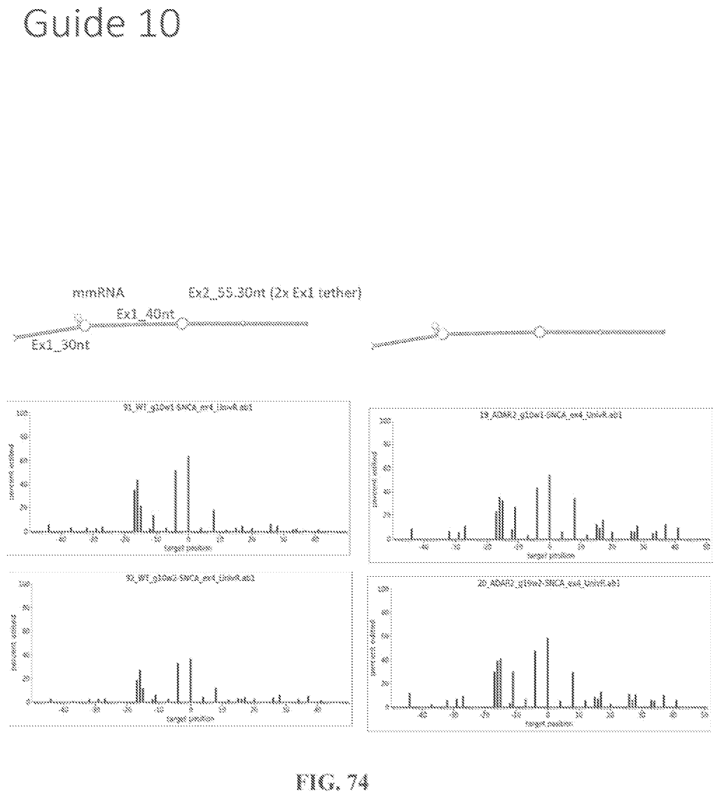

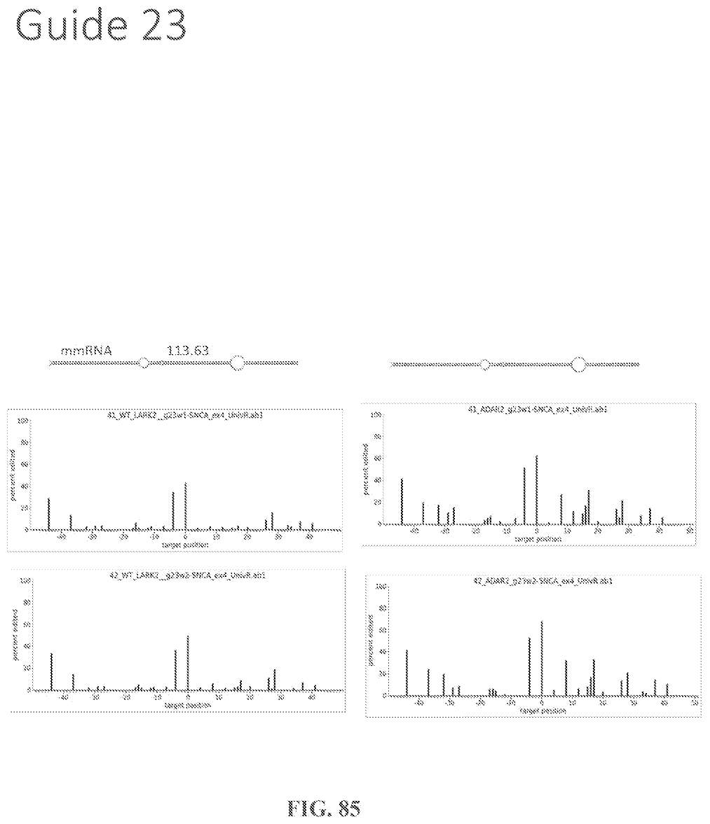

FIG. 52-FIG. 94 show plots of RNA editing at the target A to be edited (“0” on the x-axis) and at RNA editing at off-target positions (represented as black bars at positions that are not “0”). Biological replicates are shown in each column. High levels of RNA editing of SNCA were observed in several guide RNA constructs. Guide constructs shown in FIG. 69, FIG. 74, and FIG. 85, corresponding to SEQ ID NO: 76, SEQ ID NO: 81, and SEQ ID NO: 92, respectively, exhibited high levels of RNA editing and a high ratio of on-target to off-target edits. Guides shown in FIG. 67-FIG. 75 are guides of the present disclosure that comprise oligo tethers, which is a segment of the guide adjacent to the targeting sequence that has non-continuous complementarity to the target strand.

Sequences of guides shown in FIG. 52-FIG. 94 are described below in TABLE 21. TABLE 21 below also describes features formed upon hybridization of a given guide to a target RNA, along with a non-latent hU7 hairpin, the percent on-target RNA editing observed, on-target editing as a percentage of total RNA editing that is on-target RNA editing, and on-target editing as a percentage of RNA editing at the target in the start site and downstream of the start site in the coding region.

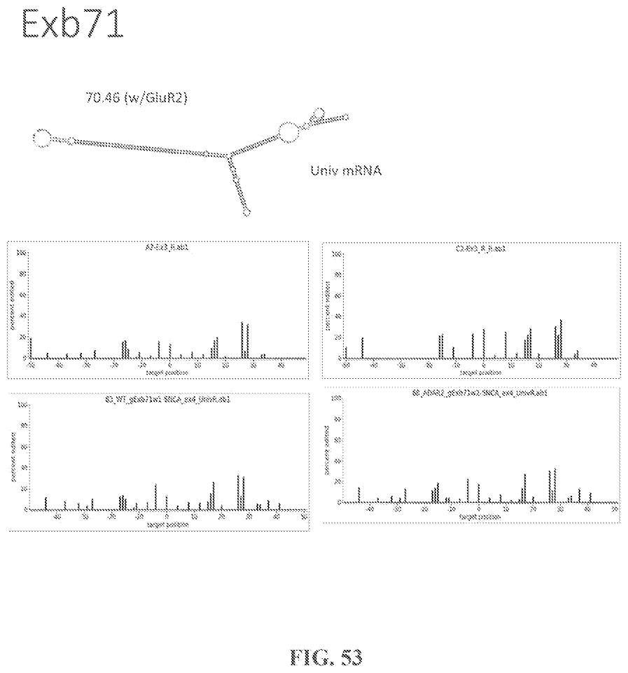

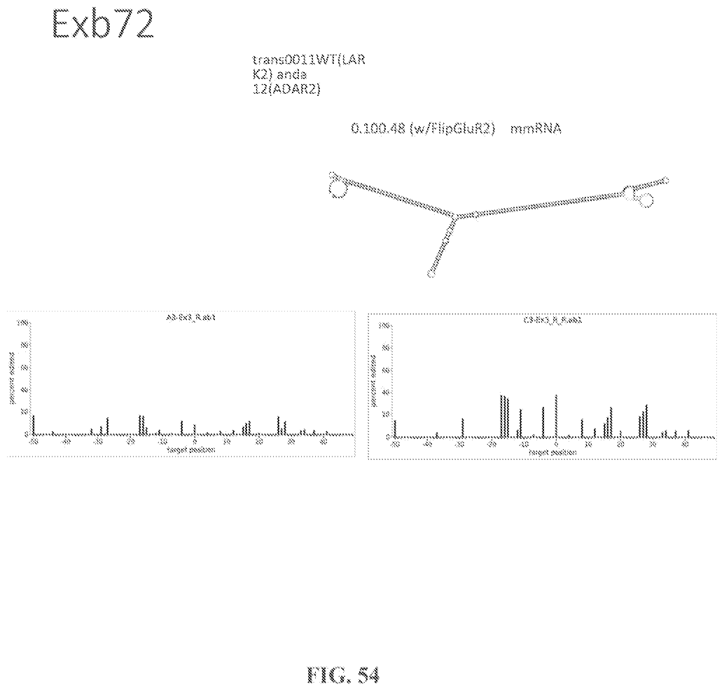

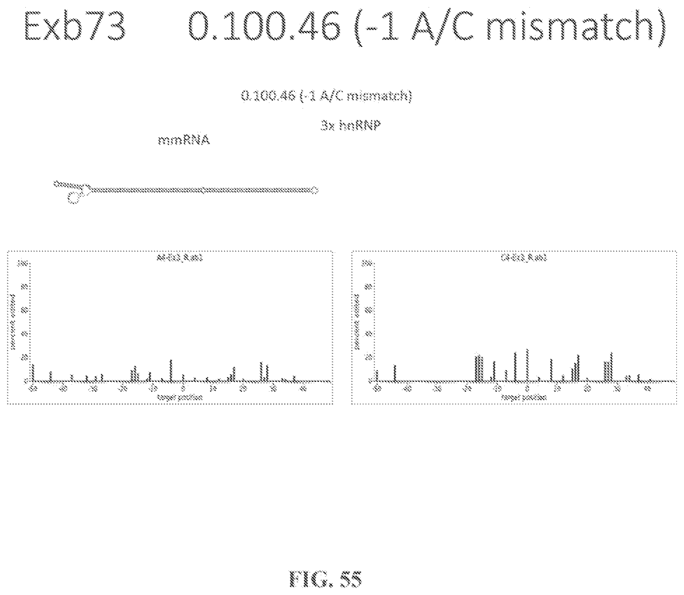

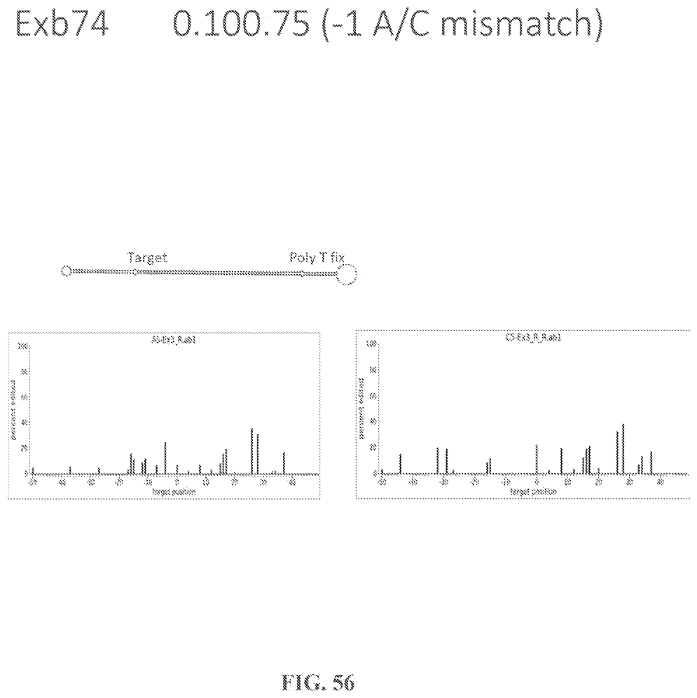

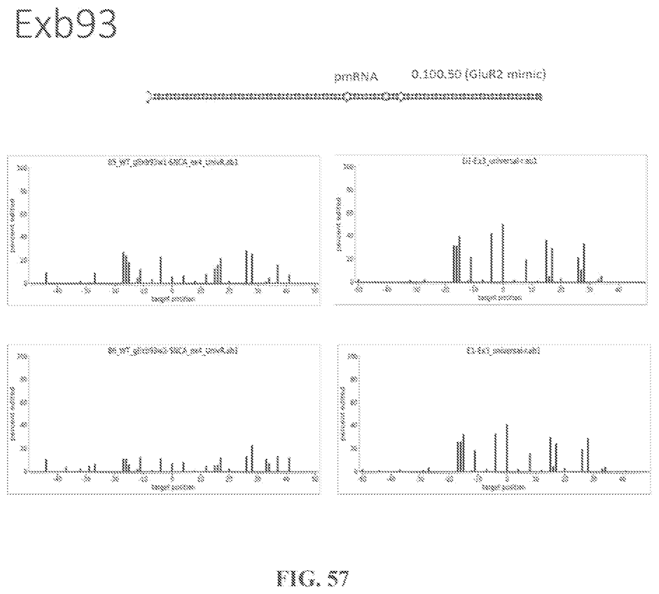

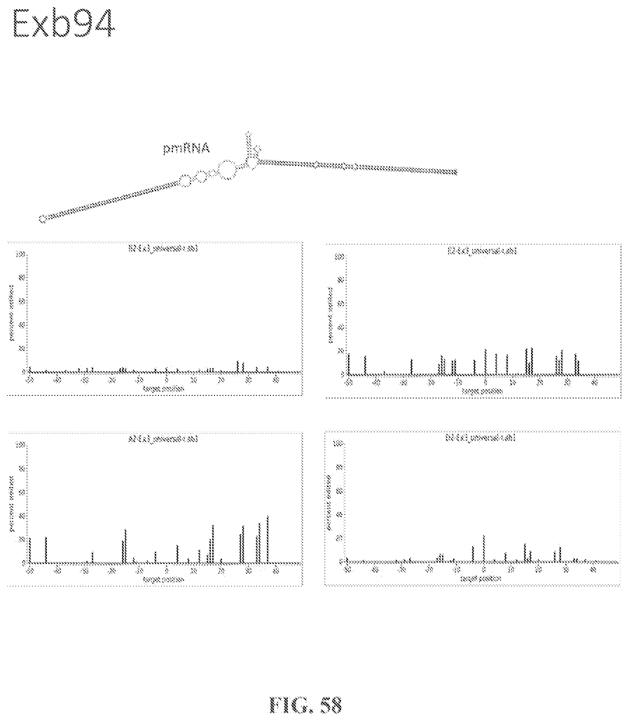

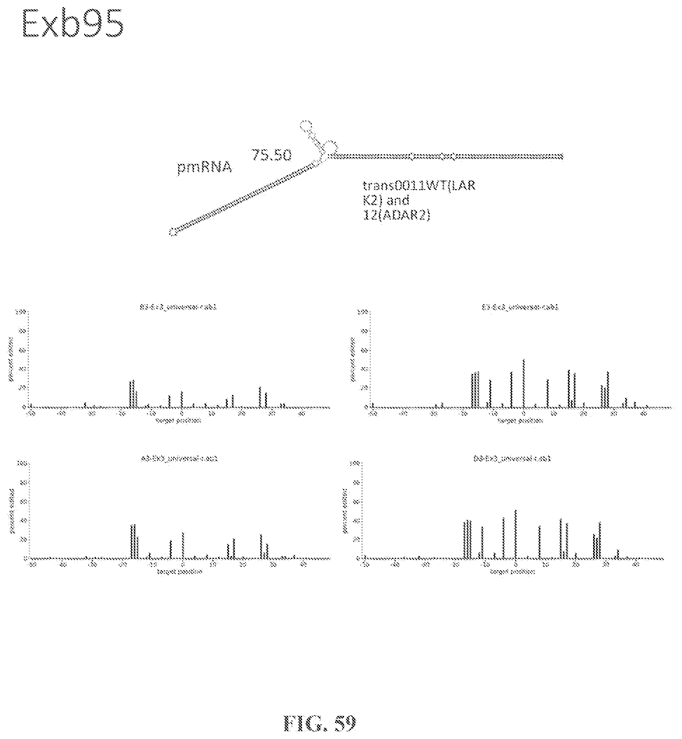

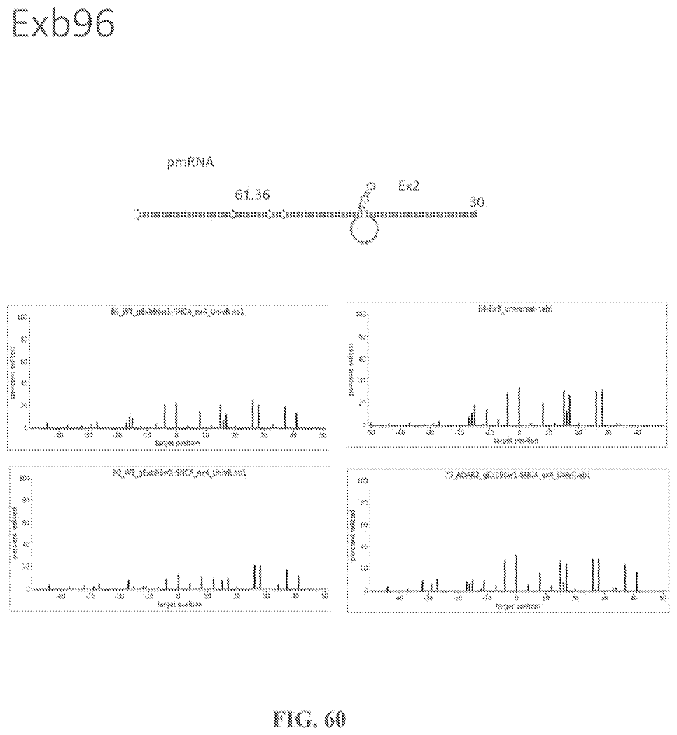

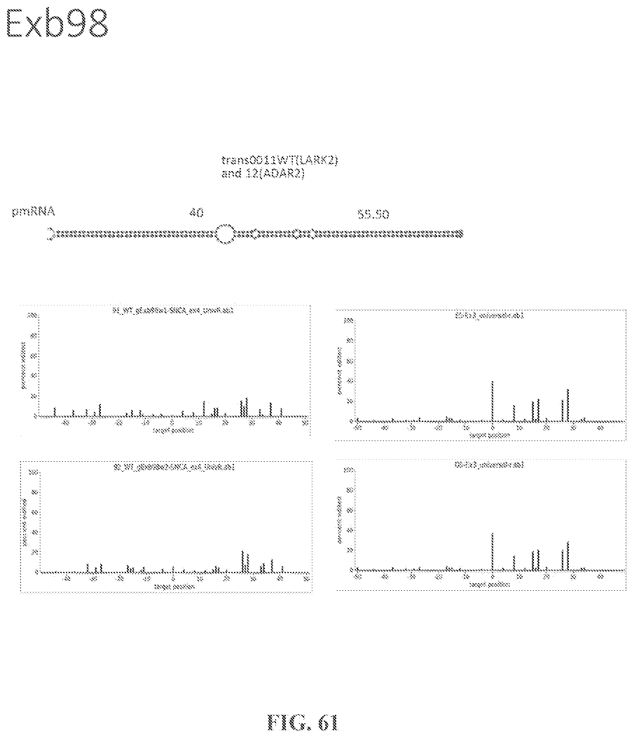

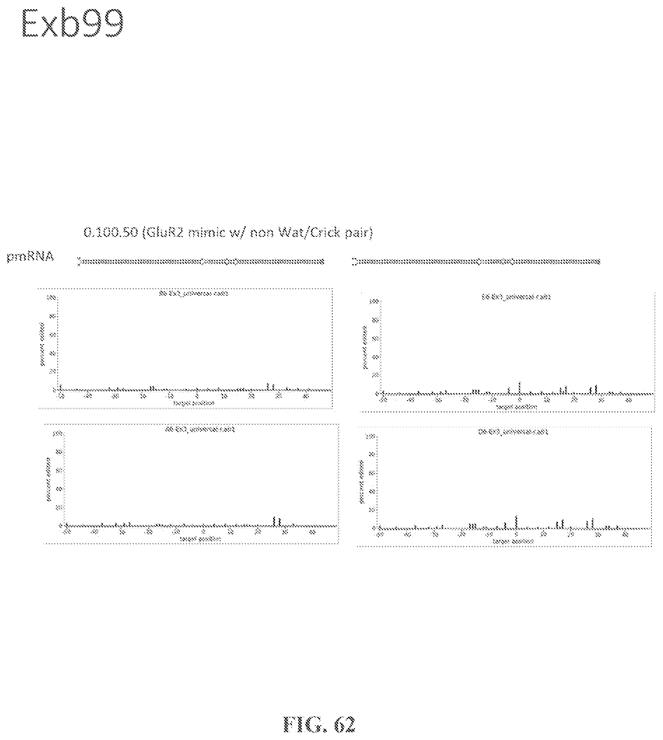

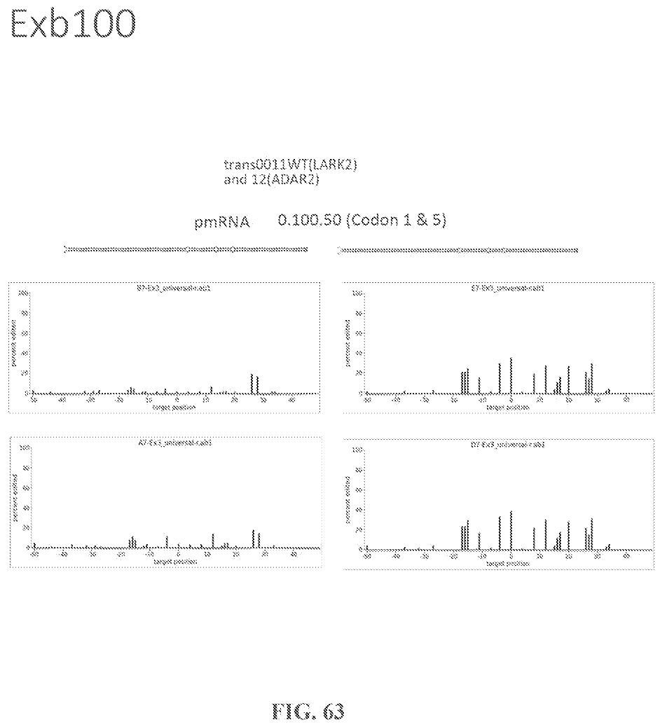

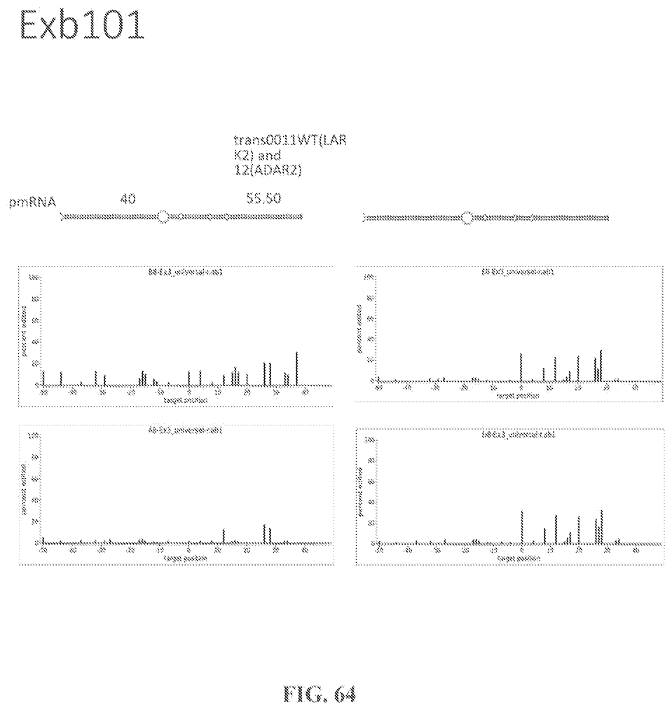

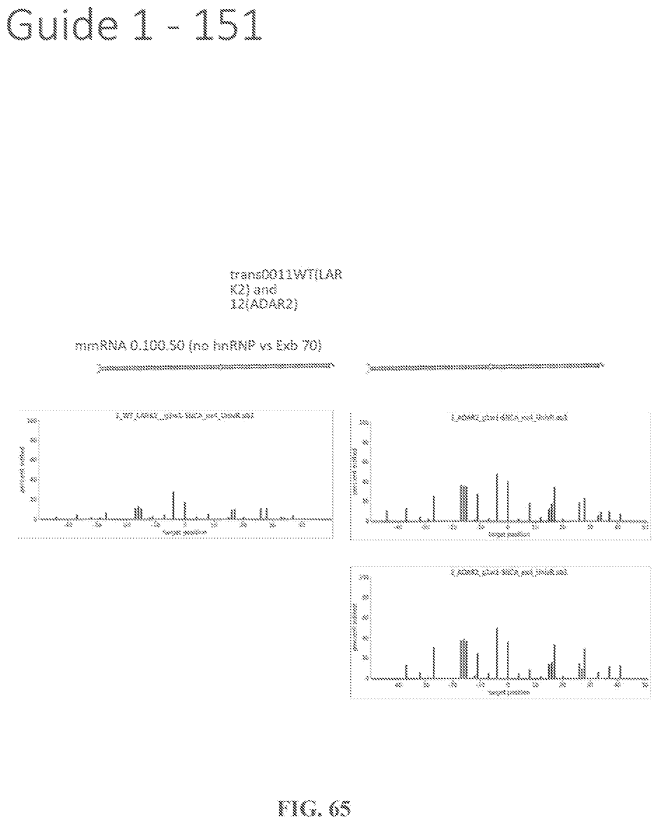

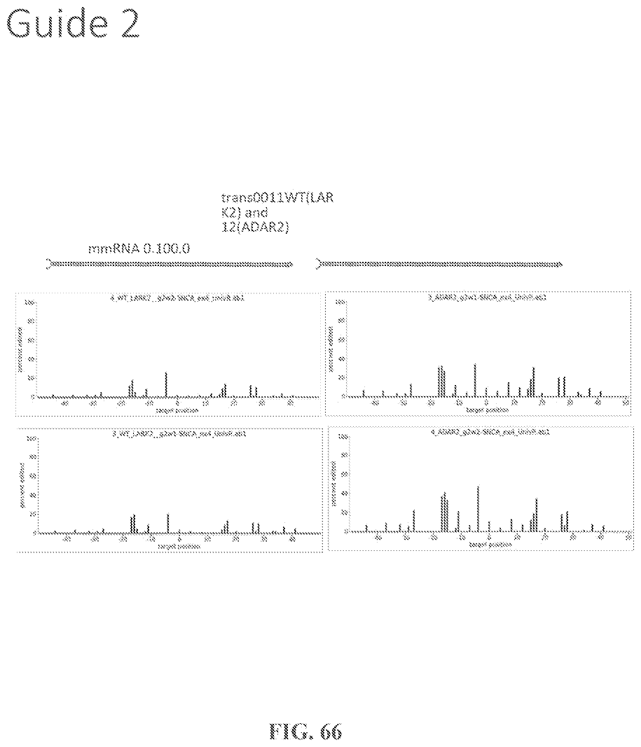

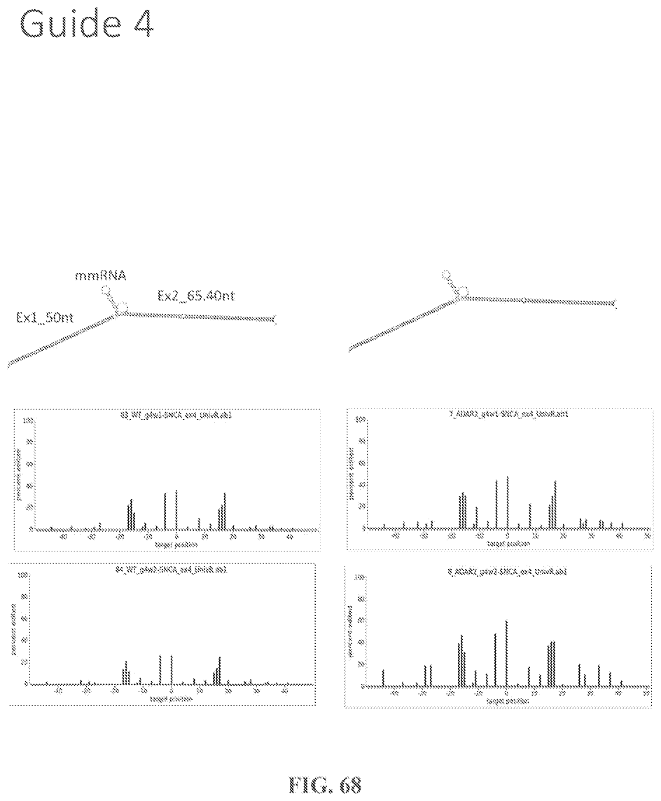

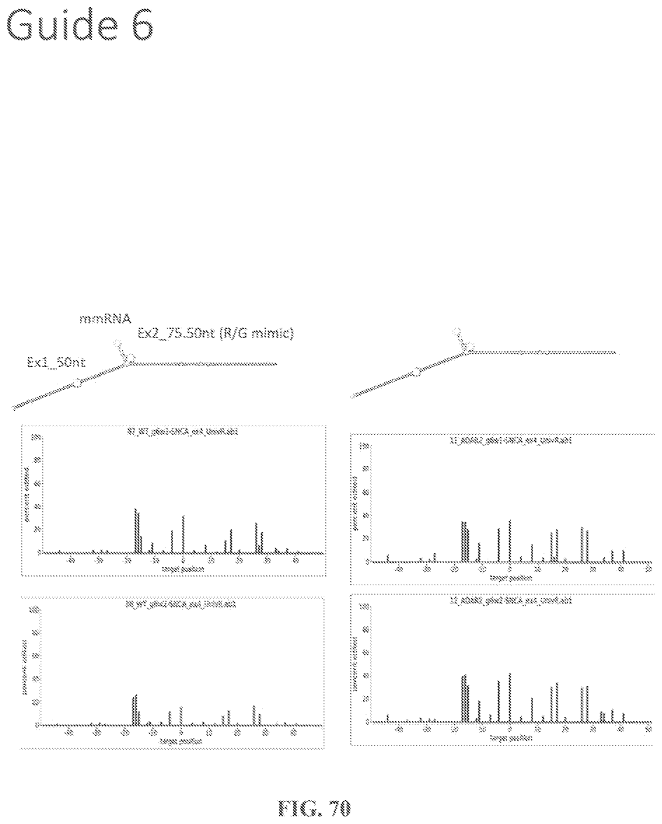

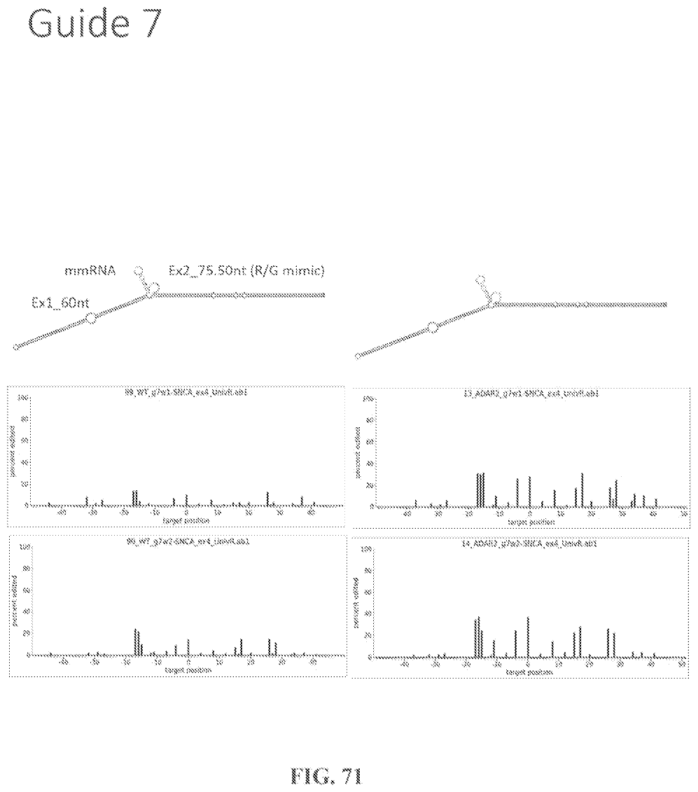

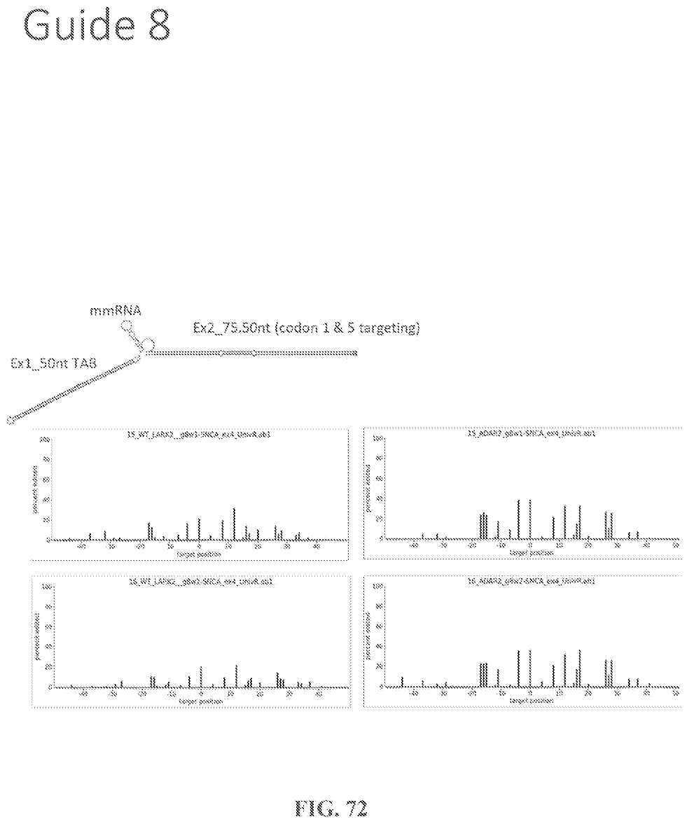

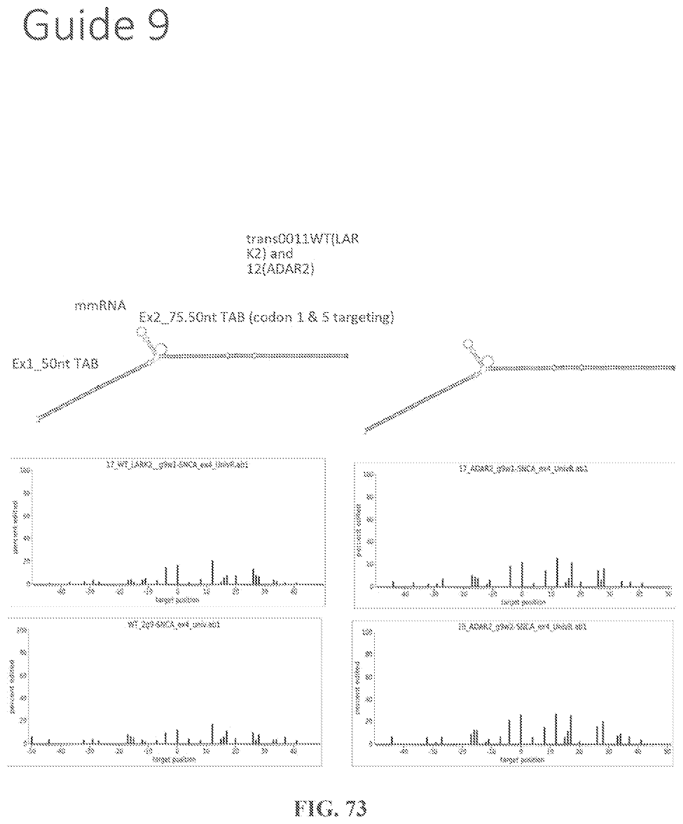

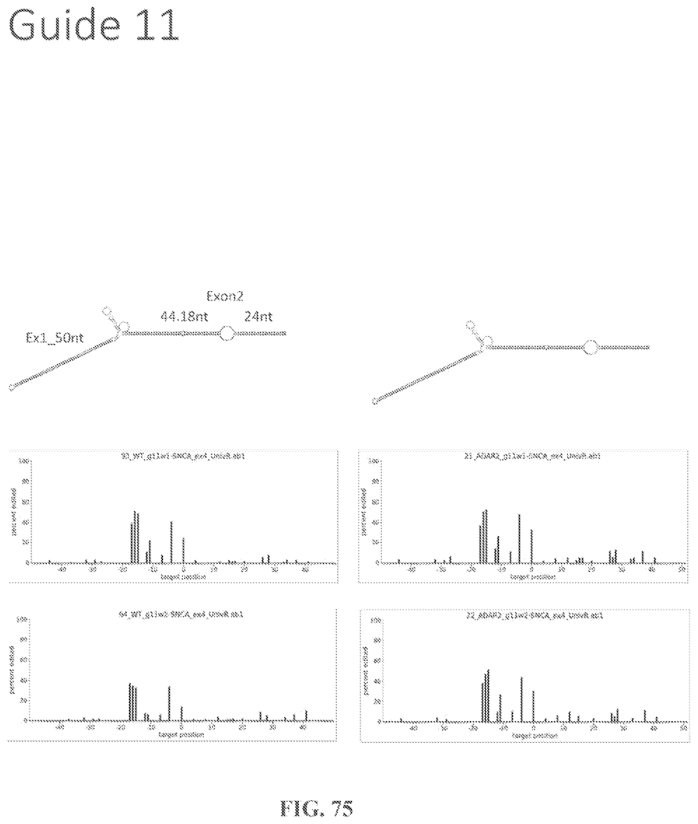

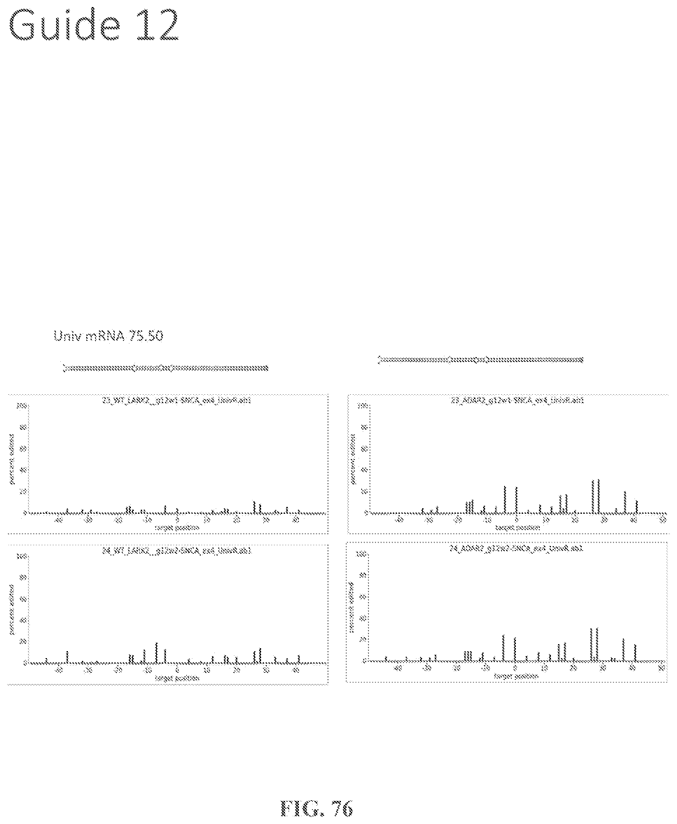

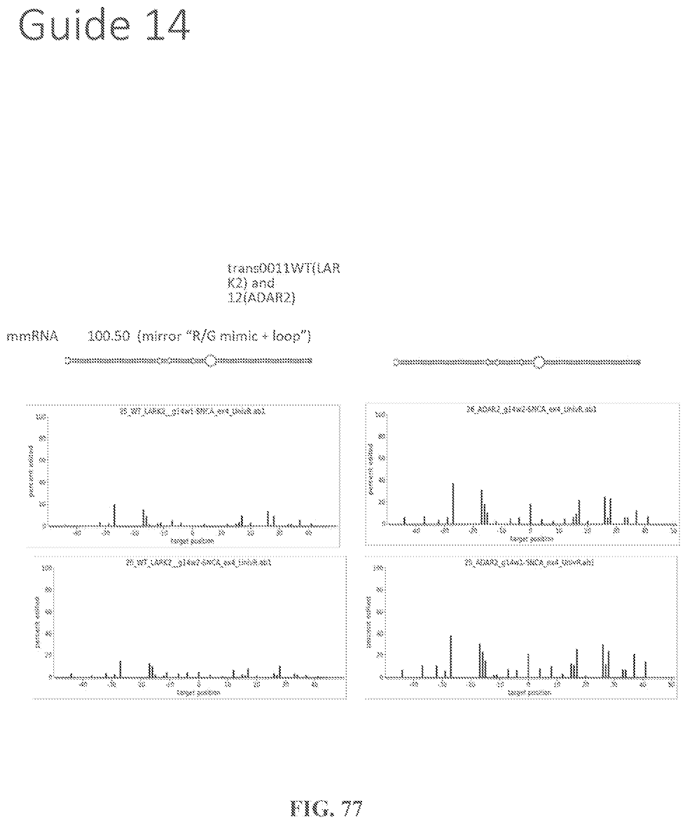

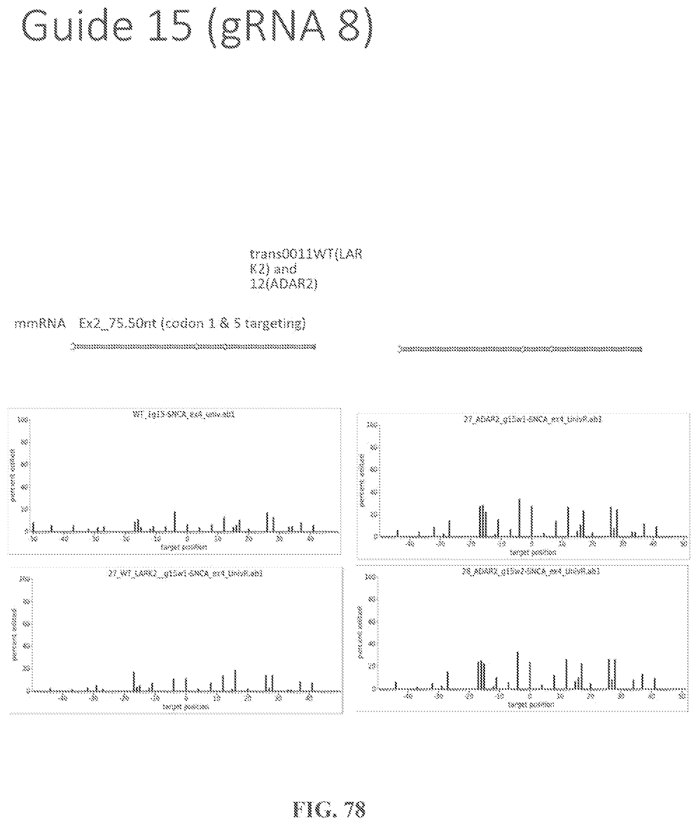

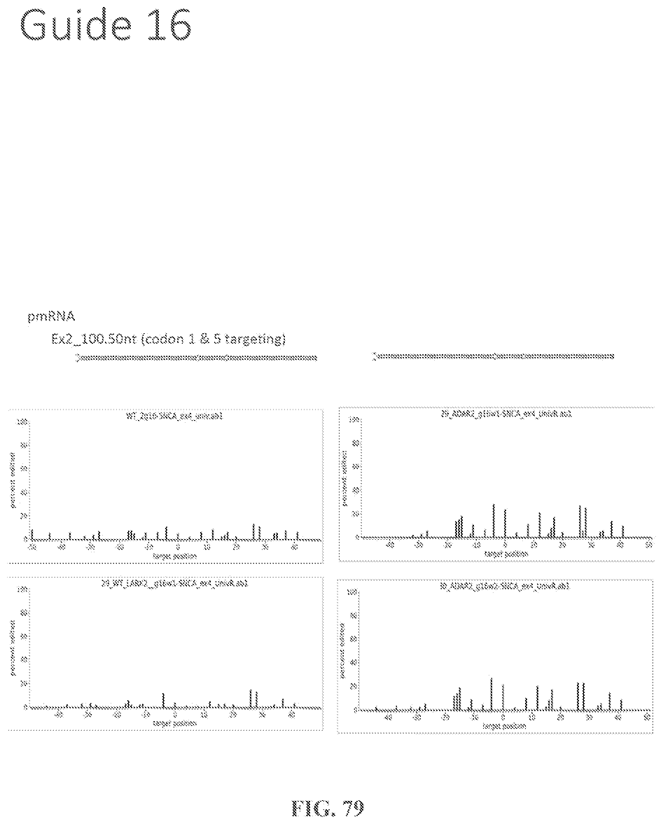

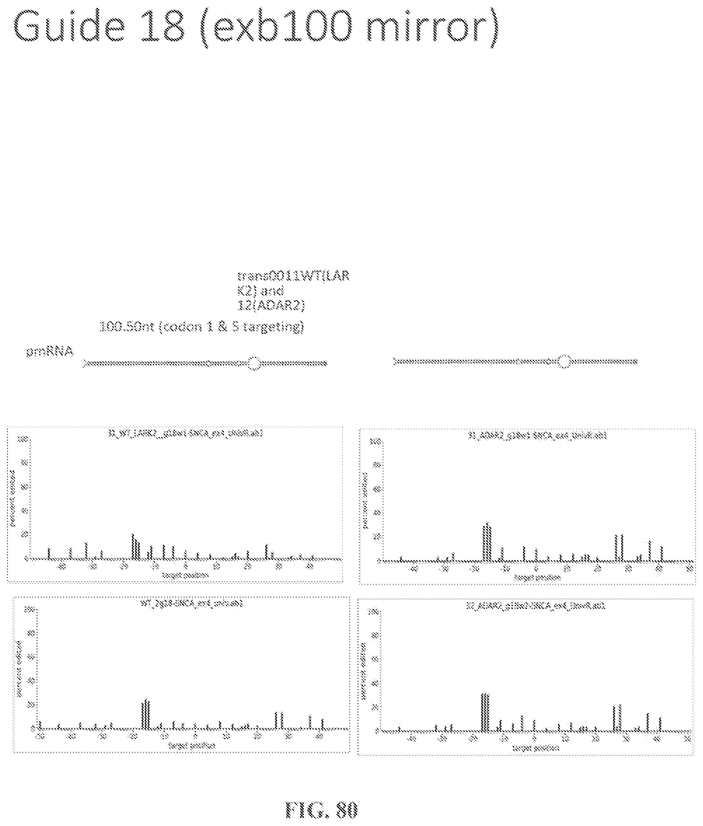

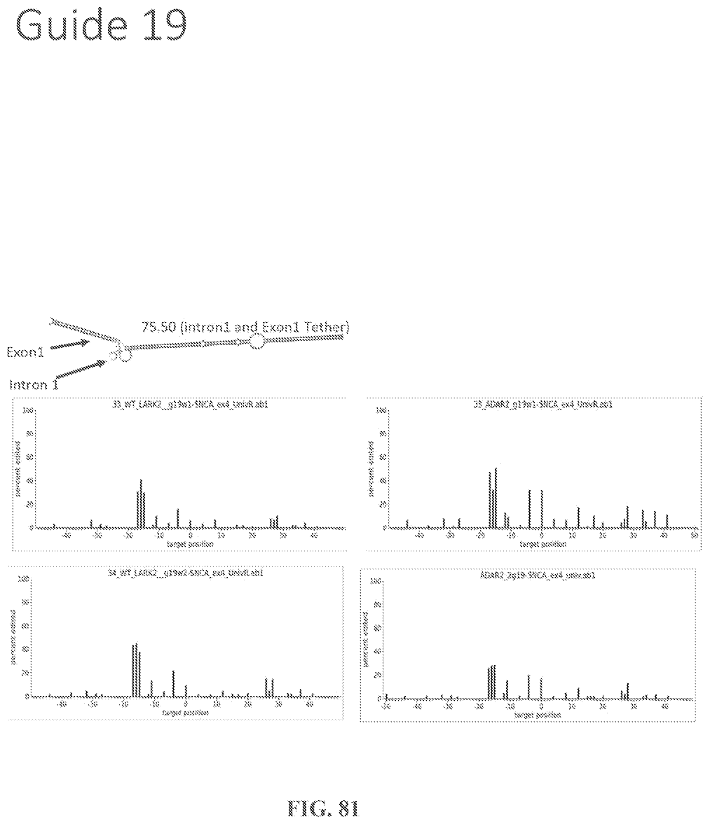

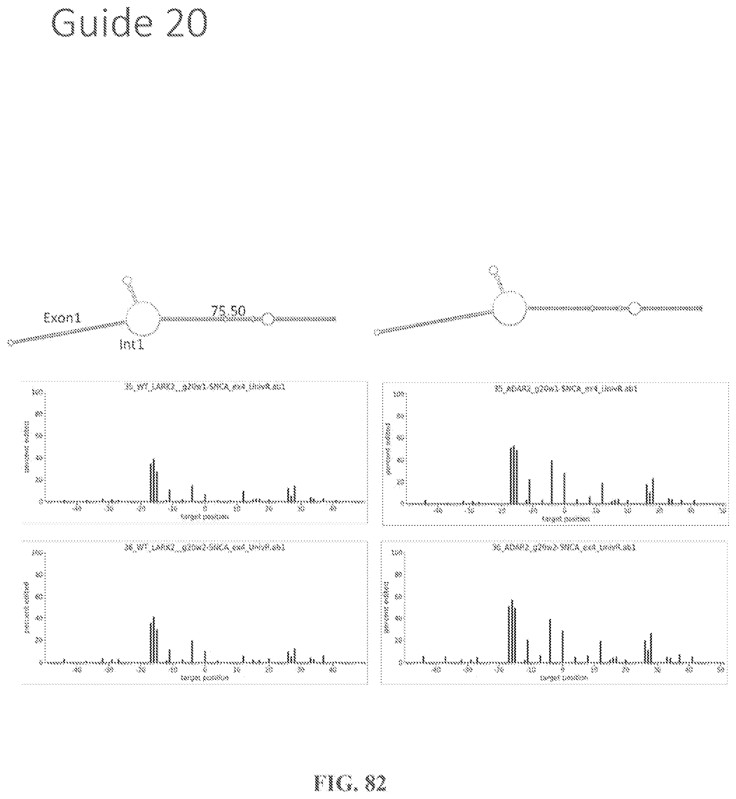

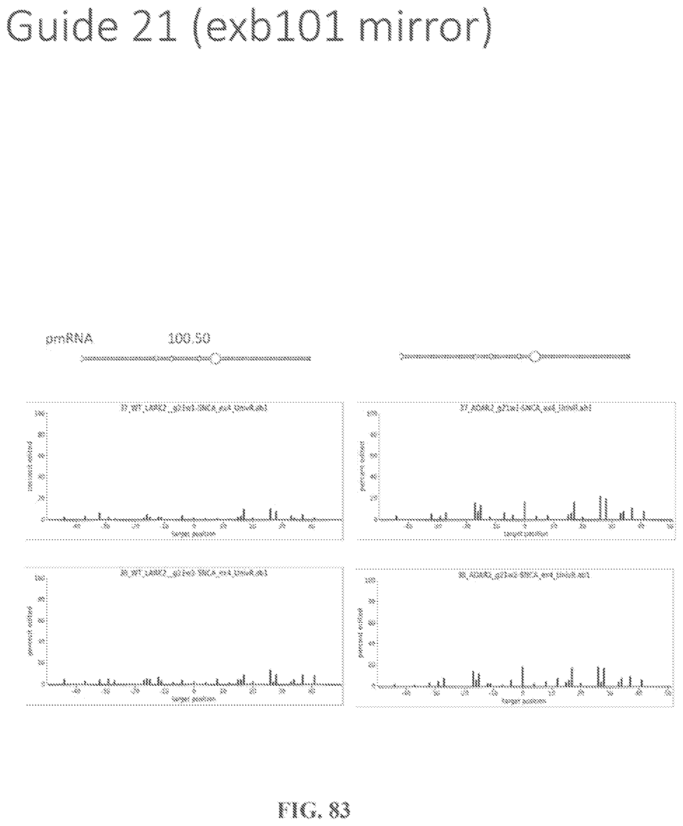

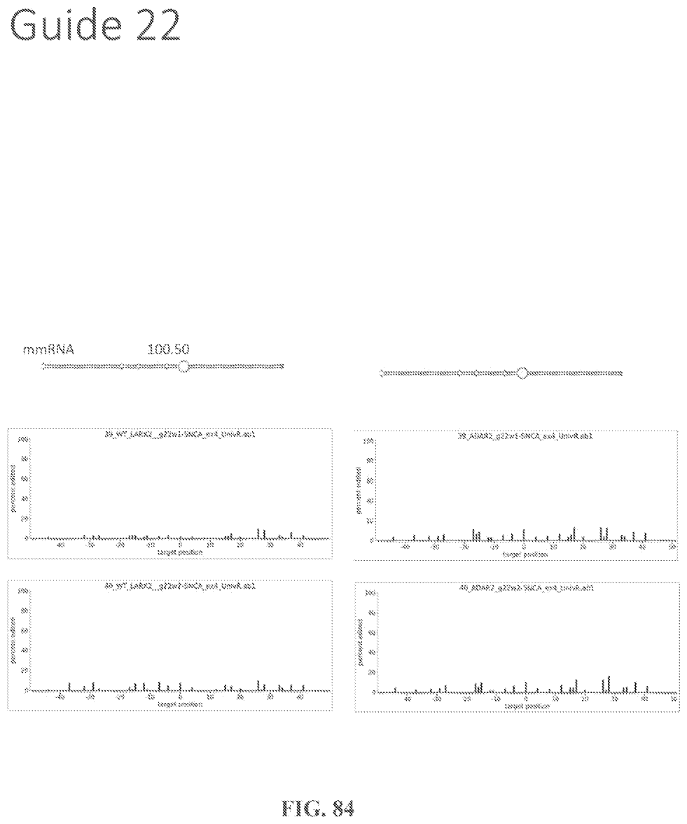

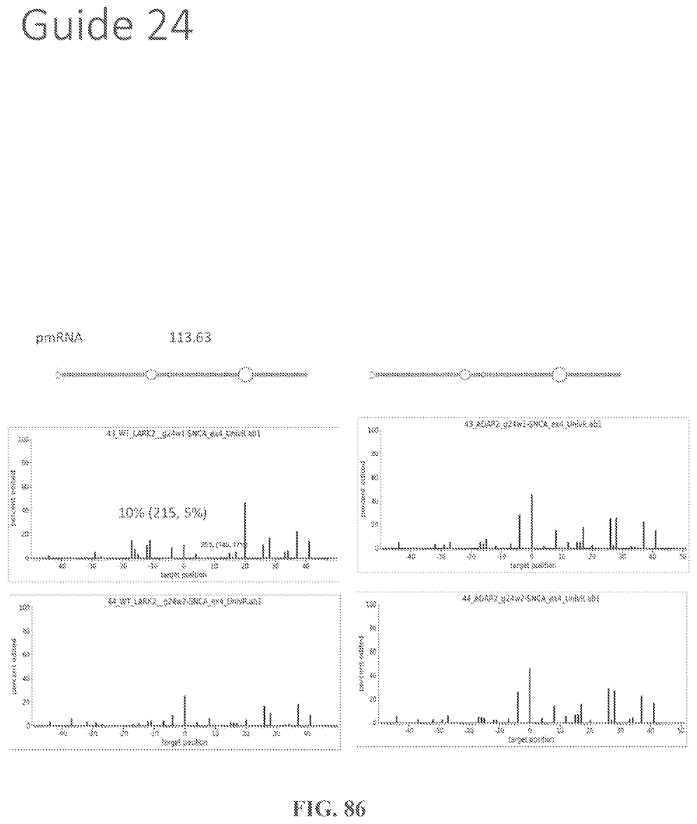

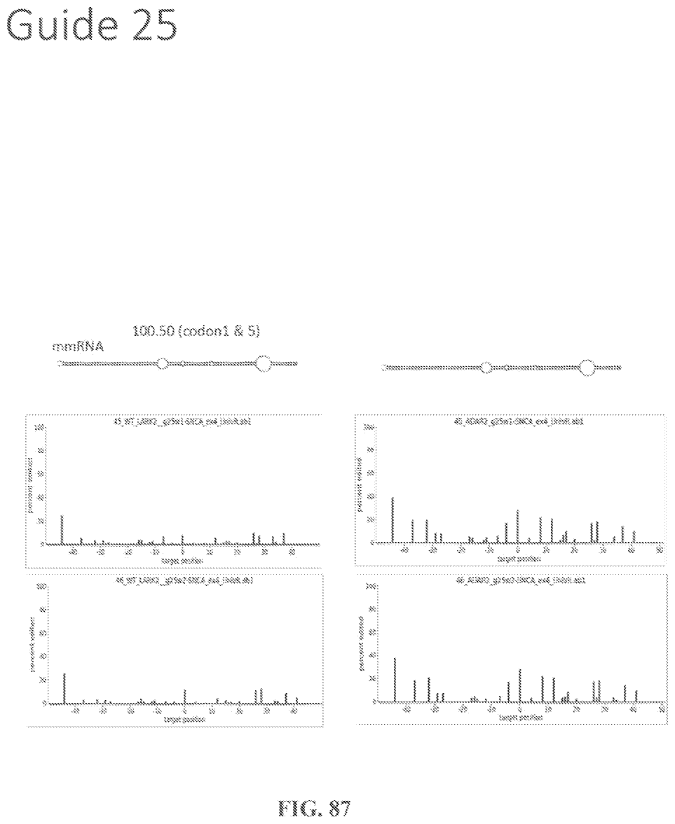

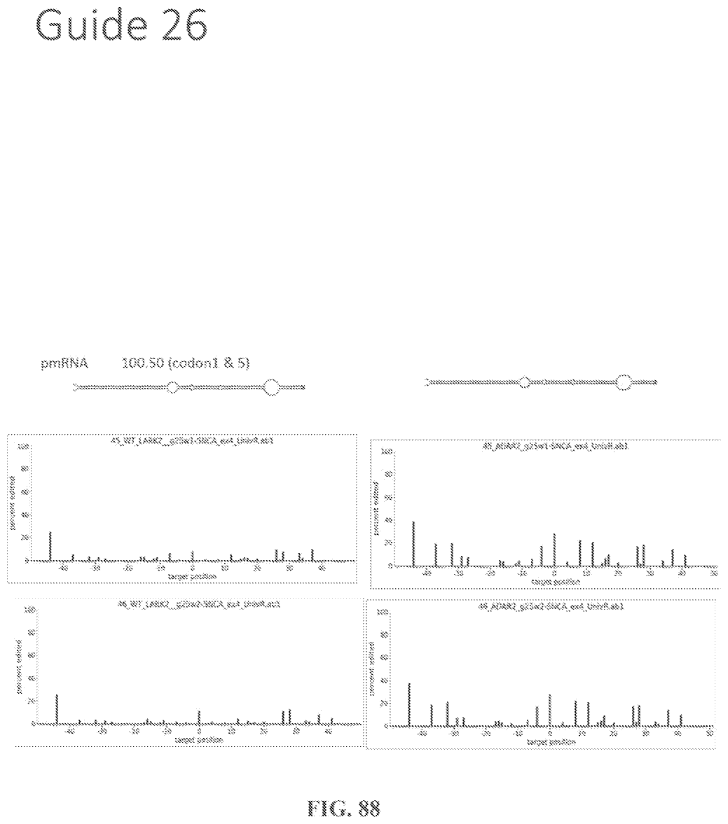

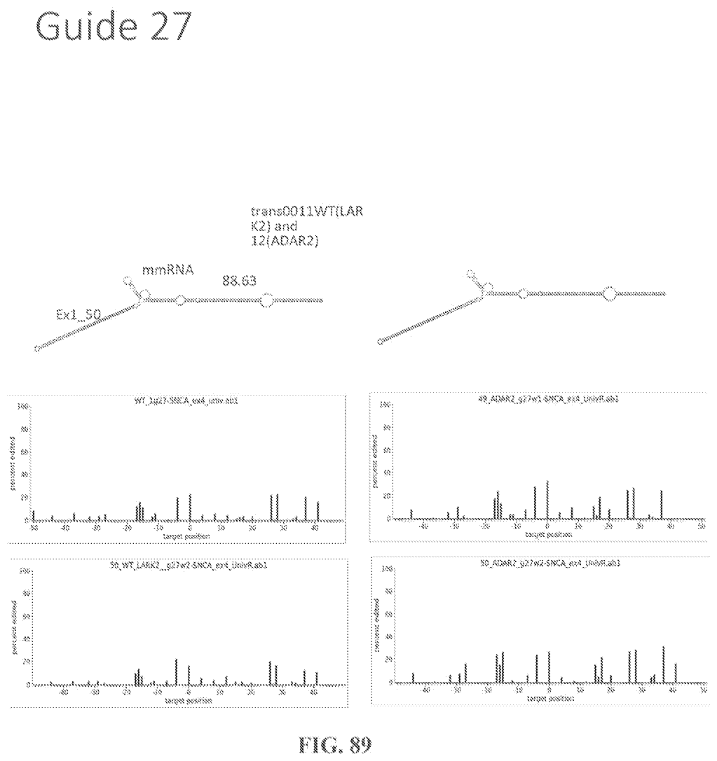

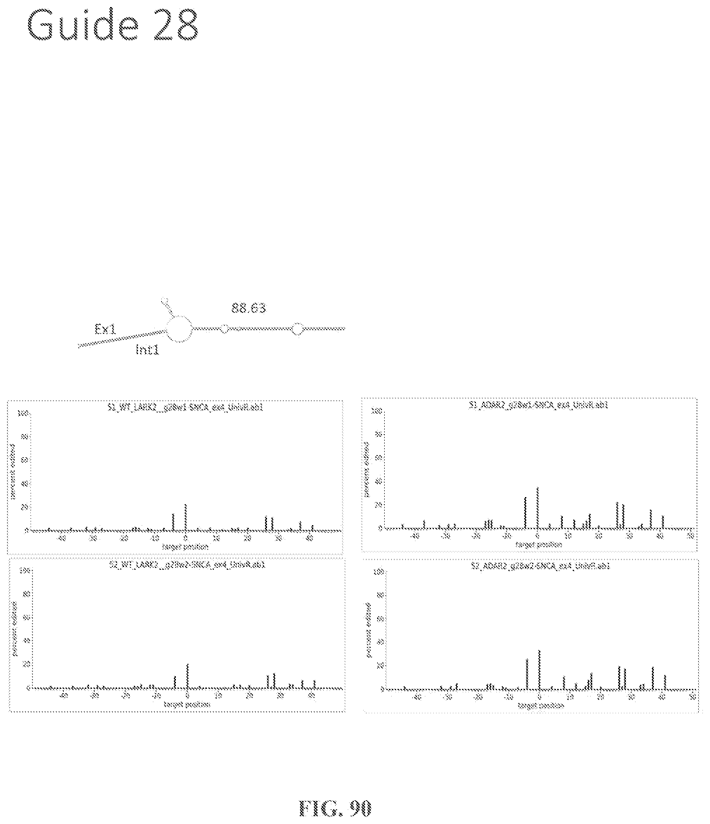

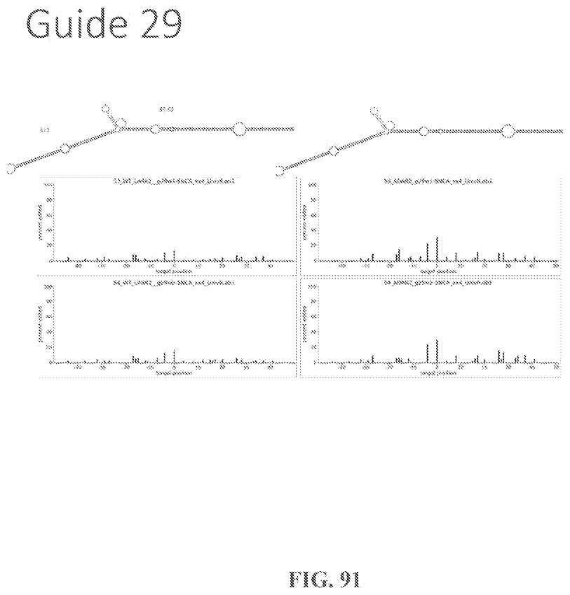

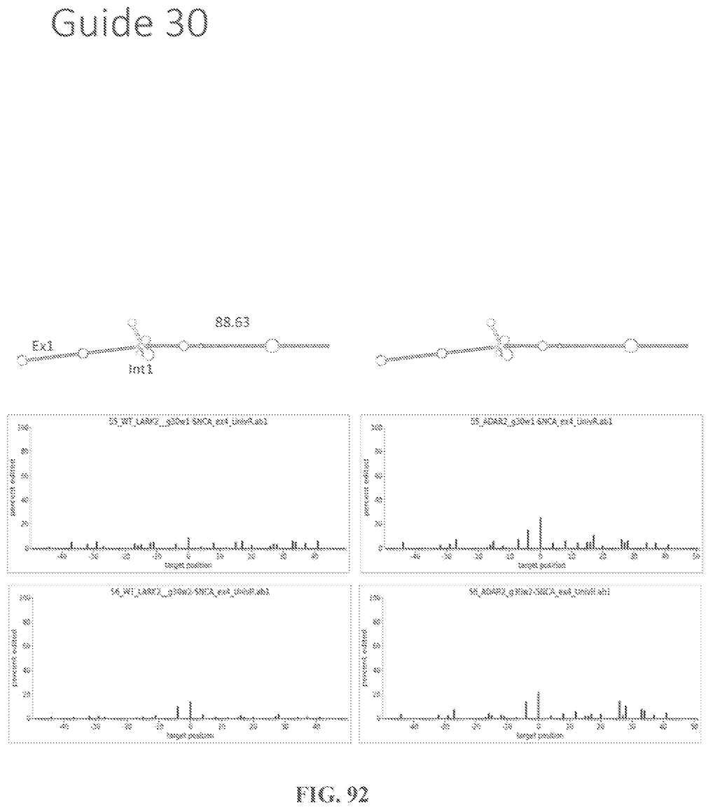

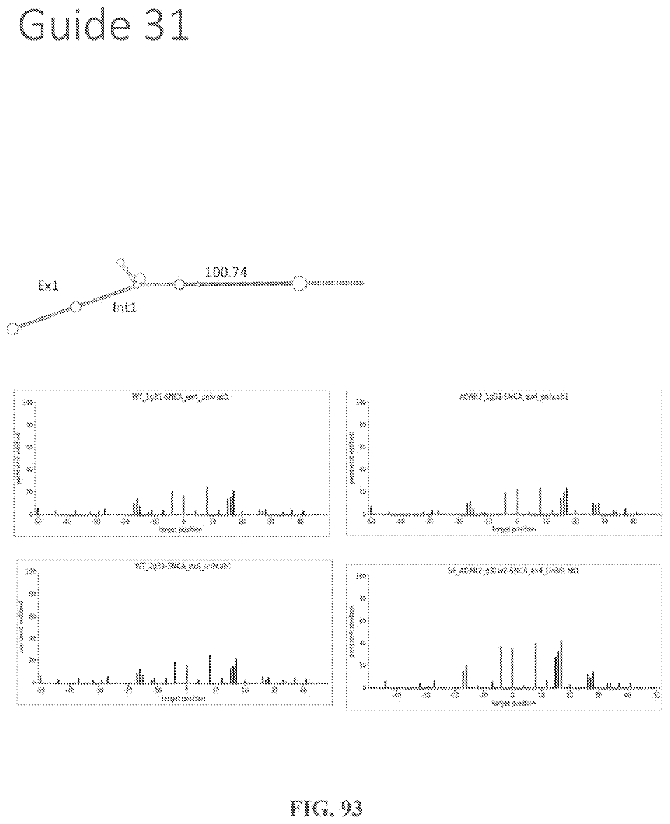

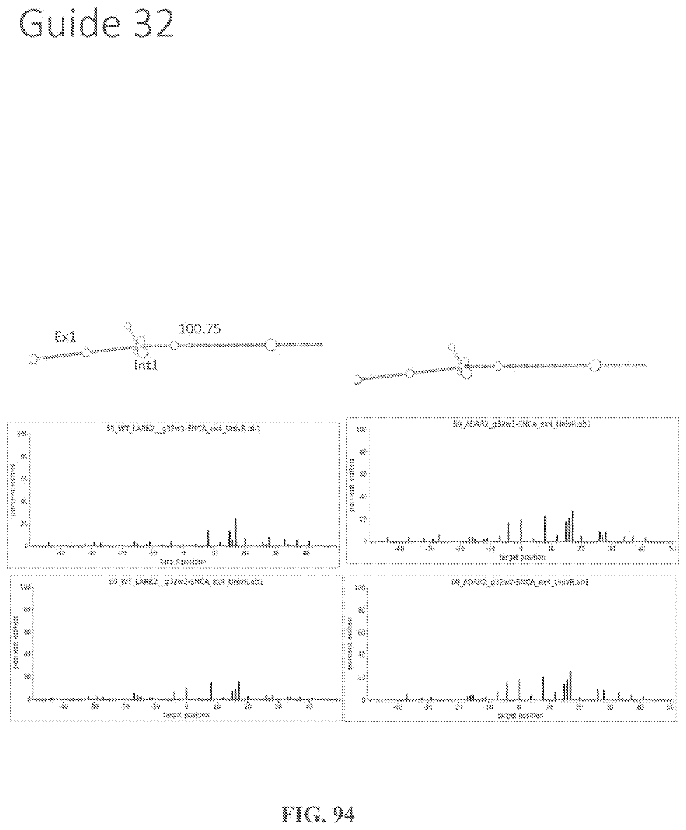

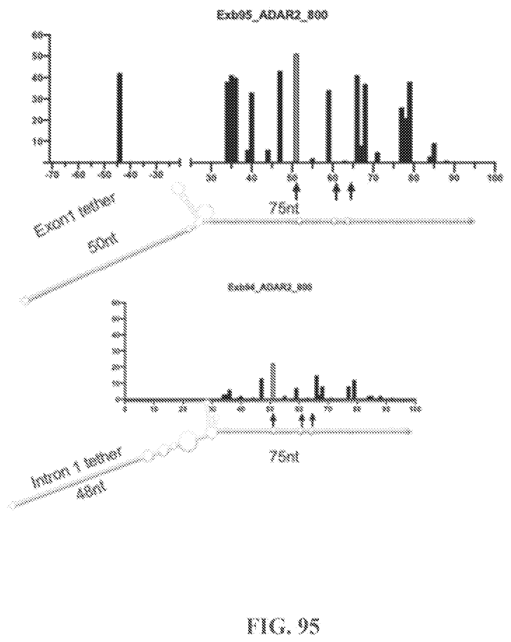

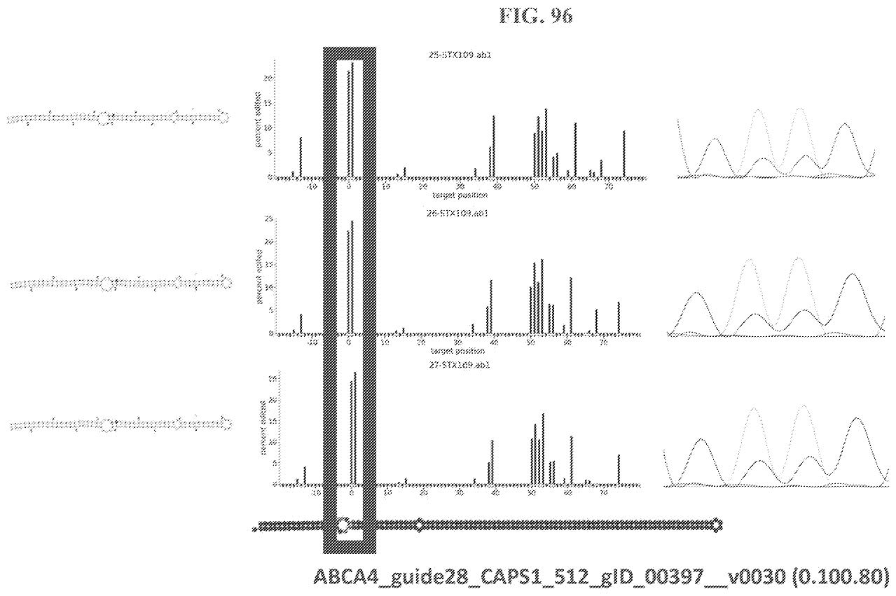

TABLE 21
Engineered Guide RNA Sequences Against SNCA

| | | | | Percent of Total |
|---|---|---|---|---|
| | | | | On-Target and |
| | | | Percent | Downstream |
| | | | On-Target | Editing that is |
| | | | Editing | On-Target |
| SEQ ID | | | [ADAR1, | [ADAR1, |
| NO | | Structural Features | ADAR1 + | ADAR1 + |
| FIG | Sequence | (target/guide) | ADAR2] | ADAR2] |
| Target | | — | — | — |
| Sequence | | | | |
| SEQ ID | TAATAGGGATAGGGATAGGGAC | 3x hnRNP, | [20, 40] | [12, 19] |
| NO: 59 | AACTCCCTCCTTGGCCTTTGAAA | A/C mismatch at 0 | | |
| FIG. 52 | GTCCTTTCATGAATACATCCACG | position SmOPT | | |
| | GCTAATGAATTCCTTTACACCAC | hU7 hairpin | | |
| | ACTGTCGTCGAATGGCCACTCCC | | | |
| | AGTTCTCATATAATTTTTGGAG | | | |
| | CAGGTTTTCTGACTTCGGTCG | | | |
| | GAAAACCCCTCC | | | |
| SEQ ID | TAATAGGGATAGGGATAGGGAC | 3x hnRNP, | [13, 23] | [7, 13.5] |
| NO: 60 | AACTCCCTCCTTGGCCTTTGAAA | A/C mismatch at 0 | | |
| FIG. 53 | GTCCTTTCATGAATACATCCACG | position | | |
| | GCTAATGTGGAATAGTATAACA | GluR2 recruitment | | |
| | ATATGCTAAATGTTGTTATAGTA | domain at −7 position | | |
| | TCCCACGAATTCCTTTACACCAC | SmOPT hU7 | | |
| | ATATAATTTTTGGAGCAGGTTT | hairpin | | |
| | TCTGACTTCGGTCGGAAAACC | | | |
| | CCTCC | | | |
| SEQ ID | TAAT_CAACTCCCTCCTTGGCCT | A/C mismatch at 0 | [10, 37] | [10,17] |
| NO: 61 | TTGAAAGTCCTTTCATGAATACA | position | | |
| FIG. 54 | CCCTATGATATTGTTGTAAATCG | flipGluR2 | | |
| | TATAACAATATGATAAGGTGCA | recruitment domain | | |
| | TCCACGGCTAATGAATTCCTTTA | at +6 position | | |
| | CACCACACTGTCGTCGAATGGC | SmOPT hU7 | | |
| | CACTCCCAGTTCTCATATAATTT | hairpin | | |
| | TTGGAGCAGGTTTTCTGACTT | | | |
| | CGGTCGGAAAACCCCTCC | | | |
| SEQ ID | TAATAGGGATAGGGATAGGGAC | 3x hnRNP | [6, 25] | [7,16] |
| NO: 62 | AACTCCCTCCTTGGCCTTTGAAA | A/C mismatch at −1 | | |
| FIG. 55 | GTCCTTTCATGAATACATCCATA | position SmOPT | | |
| | GCTAATGAATTCCTTTACACCAC | hU7 hairpin | | |
| | ACTGTCGTCGAATGGCCACTCCC | | | |
| | AGTTCTCATATAATTTTTGGAG | | | |
| | CAGGTTTTCTGACTTCGGTCG | | | |
| | GAAAACCCCTCC | | | |
| SEQ ID | TAATAGGGATAGGGATAGGGAC | 3x hnRNP, | [9, 22] | [6, 10.5] |
| NO: 63 | CCTGTTTGGTTCTCTCAGCAGCA | A/C mismatch at −1 | | |
| FIG. 56 | GCCACAACTCCCTCCTTGGCCTT | position | | |
| | TGAAAGTCCTTTCATGAATACAT | 1/1 mismatch at +62 | | |
| | CCATAGCTAATGAATTCCTTTAC | position | | |
| | ACCACATATAATTTTTGGAGCA | SmOPT hU7 | | |
| | GGTTTTCTGACTTCGGTCGGA | hairpin | | |
| | AAACCCCTCC | | | |
| SEQ ID | TAATGCCACAACTCCCTCCTTGG | A/C mismatch at 0 | [3, 45] | [5, 23] |
| NO: 64 | CCTTTGAAAGTCCTTTGATGCAT | position | | |
| FIG. 57 | ACATCCACGGCTAATGAATTCCT | 1/1 mismatch at +10 | | |
| | TTACACCACACTGGAAAACATA | position | | |
| | AAATACACTTTGATATAATTTTT | 1/1 mismatch at +14 | | |
| | GGAGCAGGTTTTCTGACTTCG | position | | |
| | GTCGGAAAACCCCTCC | SmOPT hU7 | | |
| | | hairpin | | |
| SEQ ID | TAATGCCACAACTCCCTCCTTGG | A/C mismatch at 0 | [3, 21] | [3, 18] |
| NO: 65 | CCTTTGAAAGTCCTTTGATGCAT | position | | |
| FIG. 58 | ACATCCACGGCTAATGAATTCCT | 1/1 mismatch at +10 | | |
| | TTACAGGAGAGTCAATTATGAT | position | | |
| | ATTATAGAGTTTGACTGAAAAA | 1/1 mismatch at +14 | | |
| | TCATGAGAAACCCTCCAAAGTG | position | | |
| | TGAGACTCTTTTAAGTGTATATA | 65/31 asymmetric | | |
| | ATTTTTGGAGCAGGTTTTCTG | loop at −21 position | | |
| | ACTTCGGTCGGAAAACCCCTC | SmOPT hU7 | | |
| | C | hairpin | | |
| SEQ ID | TAATGCCACAACTCCCTCCTTGG | A/C mismatch at 0 | [22, 50] | [18, 18] |
| NO: 66 | CCTTTGAAAGTCCTTTGATGCAT | position | | |
| FIG. 59 | ACATCCACGGCTAATGAATTCCT | 1/1 mismatch at +10 | | |
| | TTACACCACATTAGCCAGAAGG | position | | |
| | CTTGAAGGCAAGGCGTGAGGGA | 1/1 mismatch at +14 | | |
| | GCGCCCAGGACGCTCTCGGAGA | position | | |
| | TATAATTTTTGGAGCAGGTTTT | 49/4 asymmetric loop | | |
| | CTGACTTCGGTCGGAAAACCC | at −26 position | | |
| | CTCC | SmOPT hU7 | | |
| | | hairpin | | |
| SEQ ID | TAATGCTTCTGCCACACCCTGTT | A/C mismatch at 0 | [18, 37] | [12, 15] |
| NO: 67 | TGGTTTTCTCAAAGGGTATGAA | position | | |
| FIG. 60 | GAAGGGGAGCTTGGCCTTTGAA | 1/1 mismatch at +10 | | |
| | AGTCCTTTGATGCATACATCCAC | position | | |
| | GGCTAATGAATTCCTTTACACCA | 1/1 mismatch at +14 | | |
| | CATATAATTTTTGGAGCAGGTT | position | | |
| | TTCTGACTTCGGTCGGAAAAC | 20/20 symmetric loop | | |
| | CCCTCC | at +35 position | | |
| | | SmOPT hU7 | | |
| | | hairpin | | |
| SEQ ID | TAATGCCACAACTCCCTCCTTGG | A/C mismatch at 0 | [3, 38] | [5, 24] |
| NO: 68 | CCTTTGAAAGTCCTTTGATGCAT | position | | |
| FIG. 61 | ACATCCACGGCTATACTTTTCCT | 1/1 mismatch at +10 | | |
| | TTACACCACACTGGAAAACATA | position | | |
| | AAATACACTTTGATATAATTTTT | 1/1 mismatch at +14 | | |
| | GGAGCAGGTTTTCTGACTTCG | position | | |
| | GTCGGAAAACCCCTCC | 6/6 symmetric loop at | | |
| | | −6 position | | |
| | | SmOPT hU7 | | |
| | | hairpin | | |
| SEQ ID | TAATGCCACAACTCCCTCCTTGG | A/C mismatch at 0 | [1.5, 13] | [5, 21] |
| NO: 69 | CCTTTGAAAGTCCTTTGATGCAT | position | | |
| FIG. 62 | ATATTTACGGCTAATGAATTCCT | 2/2 Wobble pairs at | | |
| | TTACACCACACTGGAAAACATA | +2 to +3 positions | | |
| | AAATACACTTTGATATAATTTTT | 1/1 mismatch at +10 | | |
| | GGAGCAGGTTTTCTGACTTCG | position | | |
| | GTCGGAAAACCCCTCC | 1/1 mismatch at +14 | | |
| | | position | | |
| | | SmOPT hU7 | | |
| | | hairpin | | |
| SEQ ID | TAATGCCACAACTCCCTCCTTGG | A/C mismatch at 0 | [5, 37] | [5, 17] |
| NO: 70 | CCTTTGAAAGTGCTTTCACGAAT | position | | |
| FIG. 63 | ACATCCACGGCTAATGAATTCCT | 1/1 mismatch at +12 | | |
| | TTACACCACACTGGAAAACATA | position | | |
| | AAATACACTTTGATATAATTTTT | 1/1 mismatch at +19 | | |
| | GGAGCAGGTTTTCTGACTTCG | position | | |
| | GTCGGAAAACCCCTCC | SmOPT hU7 | | |
| | | hairpin | | |
| SEQ ID | TAATGCCACAACTCCCTCCTTGG | A/C mismatch at 0 | [7, 29] | [5, 16] |
| NO: 71 | CCTTTGAAAGTGCTTTCACGAAT | position | | |
| FIG. 64 | ACATCCACGGCTATACTTTTCCT | 1/1 mismatch at +12 | | |
| | TTACACCACACTGGAAAACATA | position | | |
| | AAATACACTTTGATATAATTTTT | 1/1 mismatch at +19 | | |
| | GGAGCAGGTTTTCTGACTTCG | position | | |
| | GTCGGAAAACCCCTCC | 6/6 symmetric loop at | | |
| | | −6 position | | |
| | | SmOPT hU7 | | |
| | | hairpin | | |
| SEQ ID | TAATCAACTCCCTCCTTGGCCTT | A/C mismatch at 0 | [7, 38] | [6, 19] |
| NO: 72 | TGAAAGTCCTTTCATGAATACAT | position | | |
| FIG. 65 | CCACGGCTAATGAATTCCTTTAC | SmOPT hU7 | | |
| | ACCACACTGTCGTCGAATGGCC | hairpin | | |
| | ACTCCCAGTTCTCATATATAAAT | | | |
| | TTTTGGAGCAGGTTTTCTGAC | | | |
| | TTCGGTCGGAAAACCCCTCC | | | |
| SEQ ID | TAATCAACTCCCTCCTTGGCCTT | No mismatches | [2, 9] | [3, 6] |
| NO: 73 | TGAAAGTCCTTTCATGAATACAT | SmOPT hU7 | | |
| FIG. 66 | CCATGGCTAATGAATTCCTTTAC | hairpin | | |
| | ACCACACTGTCGTCGAATGGCC | | | |
| | ACTCCCAGTTCTCATATATAAAT | | | |
| | TTTTGGAGCAGGTTTTCTGAC | | | |
| | TTCGGTCGGAAAACCCCTCC | | | |
| SEQ ID | TAAT_GCCACAACTCCCTCCTTG | A/C mismatch at 0 | [30, 59] | [24,19] |
| NO: 74 | GCCTTTGAAAGTCCTTTCATGAA | position | | |
| FIG. 67 | TACATCCACGGCTAATGAATTCC | 49/4 asymmetric loop | | |
| | TTTACACCACATTAGCCAGAAG | at −26 position | | |
| | GCTTGAAGGCAAGGCGTGAGGG | SmOPT hU7 | | |
| | AGCGCCCAGGACGCTCTCGGAG | hairpin | | |
| | ATATATAAATTTTTGGAGCAGG | | | |
| | TTTTCTGACTTCGGTCGGAAA | | | |
| | ACCCCTCC | | | |
| SEQ ID | TAATCCTCCTTGGCCTTTGAAAG | A/C mismatch at 0 | [31, 54] | [26, 22] |
| NO: 75 | TCCTTTCATGAATACATCCACGG | position | | |
| FIG. 68 | CTAATGAATTCCTTTACACCACA | 49/4 asymmetric loop | | |
| | TTAGCCAGAAGGCTTGAAGGCA | at −26 position | | |
| | AGGCGTGAGGGAGCGCCCAGGA | SmOPT hU7 | | |
| | CGCTCTCGGAGATATATAAATT | hairpin | | |
| | TTTGGAGCAGGTTTTCTGACT | | | |
| | TCGGTCGGAAAACCCCTCC | | | |
| SEQ ID | TAATCTTTGAAAGTCCTTTCATG | A/C mismatch at 0 | [24, 45] | [35, 32] |
| NO: 76 | AATACATCCACGGCTAATGAAT | position | | |
| FIG. 69 | TCCTTTACACCACATTAGCCAGA | 49/4 asymmetric loop | | |
| | AGGCTTGAAGGCAAGGCGTGAG | at −26 position | | |
| | GGAGCGCCCAGGACGCTCTCGG | SmOPT hU7 | | |
| | AGATATATAAATTTTTGGAGCA | hairpin | | |
| | GGTTTTCTGACTTCGGTCGGA | | | |
| | AAACCCCTCC | | | |
| SEQ ID | TAATGCCACAACTCCCTCCTTGG | A/C mismatch at 0 | [24, 39] | [23,18] |
| NO: 77 | CCTTTGAAAGTCCTTTGATGCAT | position | | |
| FIG. 70 | ACATCCACGGCTAATGAATTCCT | 1/1 mismatch at +10 | | |
| | TTACACCACATTAGCCAGAAGG | position | | |
| | CTTGAAGGCAAGGCGACAGGGA | 1/1 mismatch at +14 | | |
| | GCGCCCAGGACGCTCTCGGAGA | position | | |
| | TATATAAATTTTTGGAGCAGGT | 49/4 asymmetric loop | | |
| | TTTCTGACTTCGGTCGGAAAA | at −26 position | | |
| | CCCCTCC | 2/2 symmetric bulge | | |
| | | at −97 position | | |
| | | SmOPT hU7 | | |
| | | hairpin | | |
| SEQ ID | TAATGCCACAACTCCCTCCTTGG | A/C mismatch at 0 | [12, 32] | [18, 18] |
| NO: 78 | CCTTTGAAAGTCCTTTGATGCAT | position | | |
| FIG. 71 | ACATCCACGGCTAATGAATTCCT | 1/1 mismatch at +10 | | |
| | TTACACCACATTAGCAAAGGCA | position | | |
| | GAAGGCTTGAAGGCAAGGACAC | 1/1 mismatch at +14 | | |
| | CGGGAGCGCCCAGGACGCTCTC | position | | |
| | GGAGGGGCCGGGATATATAAAT | 49/4 asymmetric loop | | |
| | TTTTGGAGCAGGTTTTCTGAC | at −26 position | | |
| | TTCGGTCGGAAAACCCCTCC | 5/5 symmetric loop at | | |
| | | −96 to position | | |
| | | SmOPT hU7 | | |
| | | hairpin | | |
| SEQ ID | TAATGCCACAACTCCCTCCTTGG | A/C mismatch at 0 | [20, 37] | [15, 16] |
| NO: 79 | CCTTTGAAAGTCCTTTCACGAAT | position | | |
| FIG. 72 | ACATCCACGGCTAATGAATTCCT | 1/1 mismatch at +12 | | |
| | TTACACCACATTAGCCAGAAGG | position | | |
| | CTTGAAGGCAAGGCGGAGGGAG | 49/4 asymmetric loop | | |
| | CGCCCAGGACGCTCTCGGAGAT | at −26 position | | |
| | ATATAAATTTTTGGAGCAGGTT | 1/0 asymmetric bulge | | |
| | TTCTGACTTCGGTCGGAAAAC | at −97 position | | |
| | CCCTCC | SmOPT hU7 | | |
| | | hairpin | | |
| SEQ ID | TAATGCCACAACTCCCTCCTTGG | A/C mismatch at 0 | [17, 24] | [16, 15] |
| NO: 80 | CCTTTGAAAGTCCTTTCACGAAT | position | | |
| FIG. 73 | ACACCACGGCTAATGAATTCCTT | 1/0 asymmetric bulge | | |
| | TACACCACATTAGCCAGAAGGC | 1/1 mismatch at +12 | | |
| | TTGAAGGCAAGGCGGAGGGAGC | position | | |
| | GCCCAGGACGCTCTCGGAGATA | 49/4 asymmetric loop | | |
| | TATAAATTTTTGGAGCAGGTTT | at −26 position | | |
| | TCTGACTTCGGTCGGAAAACC | 1/0 asymmetric bulge | | |
| | CCTCC | at −97 position | | |
| | | SmOPT hU7 | | |
| | | hairpin | | |
| SEQ ID | TAATCTTTGAAAGTCCTTTCATG | A/C mismatch at 0 | [50, 57] | [50, 30] |
| NO: 81 | AATACATCCACGGCTAATGAAT | position | | |
| FIG. 74 | TCCTTTACACCACAAGGGGCGA | 7/5 asymmetric loop | | |
| | ATGGCCACTCCCAGTTCTCCGCT | at −26 position | | |
| | CACGAGGGTGGAAATAATTAAG | SmOPT hU7 | | |
| | GCGTGAGGGAGCGCCCAGGACG | hairpin | | |
| | CTCTCATATATAAATTTTTGGA | | | |
| | GCAGGTTTTCTGACTTCGGTC | | | |
| | GGAAAACCCCTCC | | | |
| SEQ ID | TAATGCCACAACTCCCTCCTTGG | A/C mismatch at 0 | [19, 31] | [33, 29] |
| NO: 82 | CCTTTCGCCAAAATTTCATGAAT | position | | |
| FIG. 75 | ACATCCACGGCTAATGAATTCCT | 1/1 mismatch at +12 | | |
| | TTACACCACATTAGCCAGAAGG | position | | |
| | CTTGAAGGCAAGGCGTGAGGGA | 8/8 symmetric loop at | | |
| | GCGCCCAGGACGCTCTCGGAGA | +18 position | | |
| | TATATAAATTTTTGGAGCAGGT | 49/4 asymmetric loop | | |
| | TTTCTGACTTCGGTCGGAAAA | at −26 position | | |
| | CCCCTCC | SmOPT hU7 | | |
| | | hairpin | | |
| SEQ ID | TAATGCCACAACTCCCTCCTTGG | A/C mismatch at 0 | [2, 23] | [4, 13] |
| NO: 83 | CCTTTGAAAGTCCTTTGATGCAT | position | | |
| FIG. 76 | ACATCCACGGCTAATGAATTCCT | 1/1 mismatch at +10 | | |
| | TTACACCACAATATATAAATTT | position | | |
| | TTGGAGCAGGTTTTCTGACTT | 1/1 mismatch at +14 | | |
| | CGGTCGGAAAACCCCTCC | position | | |
| | | SmOPT hU7 | | |
| | | hairpin | | |
| SEQ ID | TAATGCCACAACTCCCTCCTTGG | A/C mismatch at 0 | [2, 20] | [5, 11] |
| NO: 84 | CCTTTGAAAGTCCTTTCATGCCC | position | | |
| FIG. 78 | TGATCCACGGCTAATGACTTCAT | 5/5 symmetric loop at | | |
| | TTACACCACACTGTCGTCGAATG | 6 to 10 positions | | |
| | GCCACTCCCAGTATATATAAAT | 1/1 mismatch at −10 | | |
| | TTTTGGAGCAGGTTTTCTGAC | position | | |
| | TTCGGTCGGAAAACCCCTCC | 1/1 mismatch at −14 | | |
| | | position | | |
| | | SmOPT hU7 | | |
| | | hairpin | | |
| SEQ ID | TAATGCCACAACTCCCTCCTTGG | A/C mismatch at 0 | [11, 25] | [11, 13] |
| NO: 85 | CCTTTGAAAGTCCTTTCACGAAT | position | | |
| FIG. 78 | ACATCCACGGCTAATGAATTCCT | 1/1 mismatch at +12 | | |
| | TTACACCACACTGTCGTCGAATG | position | | |
| | GCCACTCCCAGTATATATAAAT | SmOPT hU7 | | |
| | TTTTGGAGCAGGTTTTCTGAC | hairpin | | |
| | TTCGGTCGGAAAACCCCTCC | | | |
| SEQ ID | TAATGCCACAACTCCCTCCTTGG | A/C mismatch at 0 | [4, 23] | [7, 13] |
| NO: 86 | CCTTTGAAAGTCCTTTCACGAAT | position | | |
| FIG. 79 | ACATCCACGGCTAATGAATTCCT | 1/1 mismatch at +12 | | |
| | TTACACCACACTGGAAAACATA | position | | |
| | AAATACACTTTGAATATATAAA | SmOPT hU7 | | |
| | TTTTTGGAGCAGGTTTTCTGA | hairpin | | |
| | CTTCGGTCGGAAAACCCCTCC | | | |
| SEQ ID | TAATGCCACAACTCCCTCCTTGG | A/C mismatch at 0 | [6, 9] | [12, 8] |
| NO: 87 | CCTTTGAATAACGGTTCACGAAT | position | | |
| FIG. 80 | ACATCCACGGCTAATGAATTCCT | 1/1 mismatch at +12 | | |
| | TTACACCACACTGGAAAACATA | position | | |
| | AAATACACTTTGAATATATAAA | 6/6 symmetric loop at | | |
| | TTTTTGGAGCAGGTTTTCTGA | +17 position | | |
| | CTTCGGTCGGAAAACCCCTCC | SmOPT hU7 | | |
| | | hairpin | | |
| SEQ ID | TAATGCCACAACTCCCTCCTTGG | A/C mismatch at 0 | [8, 32] | [12, 21] |
| NO: 88 | CCTTTGAATAACGGTTCACGAAT | position | | |
| FIG. 81 | ACATCCACGGCTAATGAATTCCT | 1/1 mismatch at +12 | | |
| | TTACACCACACTGGAAAACATA | position | | |
| | AAATACACTTTGACTGTCGTCGA | 6/6 symmetric loop at | | |
| | ATGGCCACTCCCAGTATATATA | +17 position | | |
| | AATTTTTGGAGCAGGTTTTCT | 0/25 asymmetric loop | | |
| | GACTTCGGTCGGAAAACCCCT | at | | |
| | CC | SmOPT hU7 | | |
| | | hairpin | | |
| SEQ ID | TAATGCCACAACTCCCTCCTTGG | A/C mismatch at 0 | [8, 28] | [12, 20] |
| NO: 89 | CCTTTGAATAACGGTTCACGAAT | position | | |
| FIG. 82 | ACATCCACGGCTAATGAATTCCT | 1/1 mismatch at +12 | | |
| | TTACACCACACTGGAAAACATA | position | | |
| | AAATACACTTTGATTAGCCAGA | 6/6 symmetric loop at | | |
| | AGGCTTGAAGGCAAGGCGTGAG | +17 position | | |
| | GGAGCGCCCAGGACGCTCTCGG | 46/28 asymmetric | | |
| | AGATATATAAATTTTTGGAGCA | loop at −29 position | | |
| | GGTTTTCTGACTTCGGTCGGA | SmOPT hU7 | | |
| | AAACCCCTCC | hairpin | | |
| SEQ ID | TAAT_GCCACAACTCCCTCCTTG | A/C mismatch at 0 | [2, 17] | [3, 13.5] |
| NO: 90 | GCCTTTGAAAGTCCTTTCATGCC | position | | |
| FIG. 83 | CTGATCCACGGCTAATGAATCC | 5/5 symmetric loop at | | |
| | CTTTAAACCACACTGGAAAACA | 6 position | | |
| | TAAAATACACTTTGA_ATATATA | 1/1 mismatch at −12 | | |
| | AATTTTTGGAGCAGGTTTTCT | position | | |
| | GACTTCGGTCGGAAAACCCCT | 1/1 mismatch at −19 | | |
| | CC | position | | |
| | | SmOPT hU7 | | |
| | | hairpin | | |
| SEQ ID | TAATGCCACAACTCCCTCCTTGG | A/C mismatch at 0 | [4.5, 10] | [8, 10] |
| NO: 90 | CCTTTGAAAGTCCTTTCATGCCC | position | | |
| FIG. 84 | TGATCCACGGCTAATGAATCCCT | 5/5 symmetric loop at | | |
| | TTAAACCACACTGGAAAACATA | 6 position | | |
| | AAATACACTTTGAATATATAAA | 1/1 mismatch at −12 | | |
| | TTTTTGGAGCAGGTTTTCTGA | position | | |
| | CTTCGGTCGGAAAACCCCTCC | 1/1 mismatch at −19 | | |
| | | position | | |
| | | SmOPT hU7 | | |
| | | hairpin | | |
| SEQ ID | TAATTTTCTCAGCAGCAGCCACA | A/C mismatch at 0 | [49, 68] | [42, 28] |
| NO: 92 | ACTCCGAGGAACCCCTTTGAAA | position | | |
| FIG. 85 | GTCCTTTCATGAATACATCCACG | 5/5 symmetric loop at | | |
| | GCTAAACTTCTCCTTTACACCAC | −7 to −11 positions | | |
| | ACTGTCGTCGAATGGCCACTCCC | 8/8 symmetric loop at | | |
| | AGTATATATAAATTTTTGGAGC | +31 position | | |
| | AGGTTTTCTGACTTCGGTCGG | SmOPT hU7 | | |
| | AAAACCCCTCC | hairpin | | |
| SEQ ID | TAATTTTCTCAGCAGCAGCCACA | A/C mismatch at 0 | [18, 46] | [16, 23] |
| NO: 93 | ACTCCGAGGAACCCCTTTGAAA | position | | |
| FIG. 86 | GTCCTTTCATGAATACATCCACG | 5/5 symmetric loop at | | |
| | GCTAAACTTCTCCTTTACACCAC | −7 to −11 positions | | |
| | ACTGGAAAACATAAAATACACT | 8/8 symmetric loop at | | |
| | TTGAATATATAAATTTTTGGAG | +31 position | | |
| | CAGGTTTTCTGACTTCGGTCG | SmOPT hU7 | | |
| | GAAAACCCCTCC | hairpin | | |
| SEQ ID | TAATGCCACAACTCCGAGGAAC | A/C mismatch at 0 | [9, 28] | [15, 18] |
| NO: 94 | CCCTTTGAAAGTCCTTTCACGAA | position | | |
| FIG. 87 | TACATCCACGGCTAAACTTCTCC | 5/5 symmetric loop at | | |
| | TTTACACCACACTGTCGTCGAAT | −7 position | | |
| | GGCCACTCCCAGTATATATAAA | 1/1 mismatch at +12 | | |
| | TTTTTGGAGCAGGTTTTCTGA | position | | |
| | CTTCGGTCGGAAAACCCCTCC | 8/8 symmetric loop at | | |
| | | +31 position | | |
| | | SmOPT hU7 | | |
| | | hairpin | | |
| SEQ ID | TAATGCCACAACTCCGAGGAAC | A/C mismatch at 0 | [3, 6] | [7, 8] |
| NO: 95 | CCCTTTGAAAGTCCTTTCACGAA | position | | |
| FIG. 88 | TACATCCACGGCTAAACTTCTCC | 5/5 symmetric loop at | | |
| | TTTACACCACACTGGAAAACAT | −7 position | | |
| | AAAATACACTTTGAATATATAA | 1/1 mismatch at +12 | | |
| | ATTTTTGGAGCAGGTTTTCTG | position | | |
| | ACTTCGGTCGGAAAACCCCTC | 8/8 symmetric loop at | | |
| | C | +31 position | | |
| | | SmOPT hU7 | | |
| | | hairpin | | |
| SEQ ID | TAATTTTCTCAGCAGCAGCCACA | A/C mismatch at 0 | [16, 32] | [16, 16] |
| NO: 96 | ACTCCGAGGAACCCCTTTGAAA | position | | |
| FIG. 89 | GTCCTTTCATGAATACATCCACG | 5/5 symmetric loop at | | |
| | GCTAAACTTCTCCTTTACACCAC | −7 position | | |
| | ATTAGCCAGAAGGCTTGAAGGC | 8/8 symmetric loop at | | |
| | AAGGCGTGAGGGAGCGCCCAGG | +31 position | | |
| | ACGCTCTCGGAGATATATAAAT | 49/4 asymmetric loop | | |
| | TTTTGGAGCAGGTTTTCTGAC | at −26 position | | |
| | TTCGGTCGGAAAACCCCTCC | SmOPT hU7 | | |
| | | hairpin | | |
| SEQ ID | TAATTTTCTCAGCAGCAGCCACA | A/C mismatch at 0 | [21, 34] | [29, 22] |
| NO: 97 | ACTCCGAGGAACCCCTTTGAAA | position | | |
| FIG. 90 | GTCCTTTCATGAATACATCCACG | 5/5 symmetric loop at | | |
| | GCTAAACTTCTCCTTTACACCAC | −7 position | | |
| | ACTGGAAAACATAAAATACACT | 8/8 symmetric loop at | | |
| | TTGATTAGCCAGAAGGCTTGAA | +31 position | | |
| | GGCAAGGCGTGAGGGAGCGCCC | 46/28 asymmetric | | |
| | AGGACGCTCTCGGAGATATATA | loop at − position | | |
| | AATTTTTGGAGCAGGTTTTCT | SmOPT hU7 | | |
| | GACTTCGGTCGGAAAACCCCT | hairpin | | |
| | CC | | | |
| SEQ ID | TAATTTTCTCAGCAGCAGCCACA | A/C mismatch at 0 | [15, 30] | [28, 27] |
| NO: 98 | ACTCCGAGGAACCCCTTTGAAA | position | | |
| FIG. 91 | GTCCTTTCATGAATACATCCACG | 5/5 symmetric loop at | | |
| | GCTAAACTTCTCCTTTACACCAC | −7 position | | |
| | ATTAGCAAAGGCAGAAGGCTTG | 8/8 symmetric loop at | | |
| | AAGGCAAGGACACCGGGAGCGC | +31 position | | |
| | CCAGGACGCTCTCGGAGGGGCC | 49/4 asymmetric loop | | |
| | GGGATATATAAATTTTTGGAGC | at −26 position | | |
| | AGGTTTTCTGACTTCGGTCGG | 5/5 symmetric loop at | | |
| | AAAACCCCTCC | −95 to position | | |
| | | SmOPT hU7 | | |
| | | hairpin | | |
| SEQ ID | TAAT_TTTCTCAGCAGCAGCCAC | A/C mismatch at 0 | [10, 25] | [29, 25] |
| NO: 99 | AACTCCGAGGAACCCCTTTGAA | position | | |
| FIG. 92 | AGTCCTTTCATGAATACATCCAC | 5/5 symmetric loop at | | |
| | GGCTAAACTTCTCCTTTACACCA | −7 position | | |
| | CACTGGAAAACATAAAATACAC | 8/8 symmetric loop at | | |
| | TTTGATTAGCAAAGGCAGAAGG | +31 position | | |
| | CTTGAAGGCAAGGACACCGGGA | 40/25 asymmetric | | |
| | GCGCCCAGGACGCTCTCGGAGG | loop at −29 position | | |
| | GGCCGGGATATATAAATTTTTG | 5/5 symmetric loop at | | |
| | GAGCAGGTTTTCTGACTTCGG | −95 position | | |
| | TCGGAAAACCCCTCC | SmOPT hU7 | | |
| | | hairpin | | |
| SEQ ID | TAATACCCTGTTTGGTTTCTCAG | 5/5 symmetric loop at | [19, 35] | [13, 14] |
| NO: 100 | CAGCAGGGGAGTGGCCCTCCTT | −7 to −11 position | | |
| FIG. 93 | GGCCTTTGAAAGTCCTTTCATGA | 8/8 symmetric loop at | | |
| | ATACATCCACGGCTAAACTTCTC | +41 position | | |
| | CTTTACACCACATTAGCAAAGG | 1/0 asymmetric bulge | | |
| | CAGAAGGCTTGAAGGCAAGGAC | at +60 position | | |
| | ACCGGGAGCGCCCAGGACGCTC | 40/25 asymmetric | | |
| | TCGGAGGGGCCGGGATATATAA | loop at −29 position | | |
| | ATTTTTGGAGCAGGTTTTCTG | 5/5 symmetric loop at | | |
| | ACTTCGGTCGGAAAACCCCTC | −95 position | | |
| | C | SmOPT hU7 | | |
| | | hairpin | | |
| SEQ ID | TAATACCCTGTTTGGTTTCTCAG | 5/5 symmetric loop at | [5, 20] | [7, 13] |
| NO: 101 | CAGCAGGGGAGTGGCCCTCCTT | −7 position | | |
| FIG. 94 | GGCCTTTGAAAGTCCTTTCATGA | 8/8 symmetric loop at | | |
| | ATACATCCACGGCTAAACTTCTC | +41 position | | |
| | CTTTACACCACACTGGAAAACA | 1/0 asymmetric bulge | | |
| | TAAAATACACTTTGATTAGCAA | at +60 position | | |
| | AGGCAGAAGGCTTGAAGGCAAG | 40/25 asymmetric | | |
| | GACACCGGGAGCGCCCAGGACG | loop at −29 position | | |
| | CTCTCGGAGGGGCCGGGATATA | 5/5 symmetric loop at | | |
| | TAAATTTTTGGAGCAGGTTTTC | −95 position SmOPT | | |
| | TGACTTCGGTCGGAAAACCCC | hU7 hairpin | | |
| | TCC | | | |

### Example 13

### Guide RNAs Against SERPINA1

This example describes constructs of the present disclosure encoding guide RNAs designed to target a SERPINA1 missense mutation (SERPINA1, G to A mutation at position 9989 yielding the SERPINA1 E342K mutation), implicated in Alpha-1 antitrypsin deficiency.

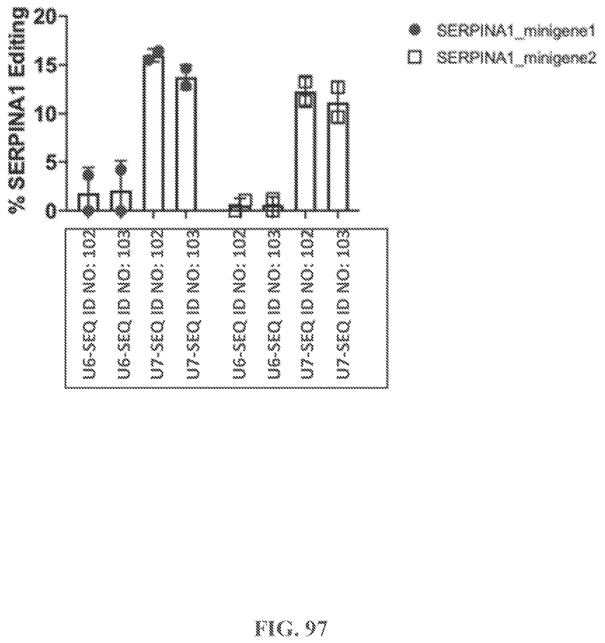

Briefly, SERPINA1 minigenes are transfected into K562 cells expressing endogenous ADAR1 via a piggyBac vector and cells were selected via puromycin selection. SERPINA1 minigenes integrated into K562 cells included a SERPINA1 minigene1 having a full length 3′ UTR or a SERPINA1 minigene2 having a truncated 3′ UTR. Both minigenes carried the G to A 9989 mutation of interest. K562 cells (2×10\hat{ }5 cells) were electroporated with plasmids encoding a guide RNA operably linked to the U7 hairpin and an SmOPT sequence. Expression was driven under a mouse U7 promoter. 24 hours post-electroporation, RNA was isolated, cDNA was synthesized via RT-PCR and RT-PCR products were sequenced via Sanger sequencing to quantify percent RNA editing. Control guide RNAs lacked the U7 hairpin and the SmOPT sequence and expression was driven under a U6 promoter. Guide RNA sequences are summarized in the table below. FIG. 97 and FIG. 98 show editing of SERPINA1 using the guide RNA sequences. Guide RNA sequences utilized include SEQ ID NO: 102 and SEQ ID NO: 103 in TABLE 22.

TABLE 22
Guide RNA Sequences

| SEQ ID NO | Sequence |
|---|---|
| SEQ ID | ACCGAUGGGUAUGGCCUCUAAAAACAUGGCCCCAGCAG |
| NO: 102 | CUUCAGUCCCUUUCUCGUCGAUGGUCAGCACAGCCUUA |
| | UGCACGGCCUGGAGGGGAGAGAAGCAGAGUGGAAUUUU |
| | UGGAGCAGGUUUUCUGACUUCGGUCGGAAAACCCCU |
| SEQ ID | ACCGAUGGGUAUGGCCUCUAAAAACAUGGCCCCAGCAG |
| NO: 103 | CUUCAGUCCCUUACUCGUCGAUGGUCAGCACAGCCUUA |
| | UGCACGGCCUGGAGGGGAGAGAAGCAGAGUGGAAUUUU |
| | UGGAGCAGGUUUUCUGACUUCGGUCGGAAAACCCCU |

### Example 14

### SNCA Editing with Circular Engineered Guide RNAs

This example describes constructs of the present disclosure encoding circular engineered guide RNAs with latent structure that manifest structural features, under the control of a U6 promoter, where the engineered guide RNA is designed to target a start site, or translation initiation site (TIS) (also referred to as translation start site (TSS)) in the SNCA gene. Engineered guide RNA constructs were designed to target SNCA TIS adenosine at nucleotide position 26 of Exon 2 (corresponding to nucleotide position 264 of most SNCA variants, including Exon 1 and Exon 2).

HEK293 cells were transfected with a plasmid encoding a guide RNA of interest and RNA editing was assessed post-transfection. RNA editing was assessed for ADAR1 only, which is naturally expressed by HEK293 cells, and ADAR1 and ADAR2. In the latter experiment, HEK293 cells were co-transfected with a piggybac vector encoding ADAR2. Levels of RNA editing were quantified by sequencing and analyzed.

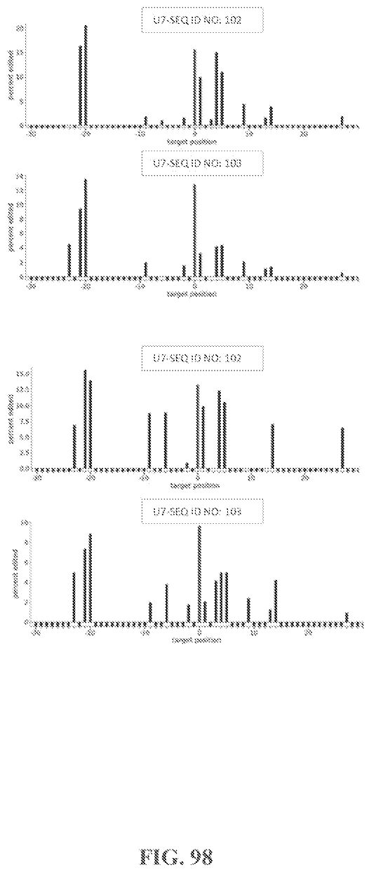

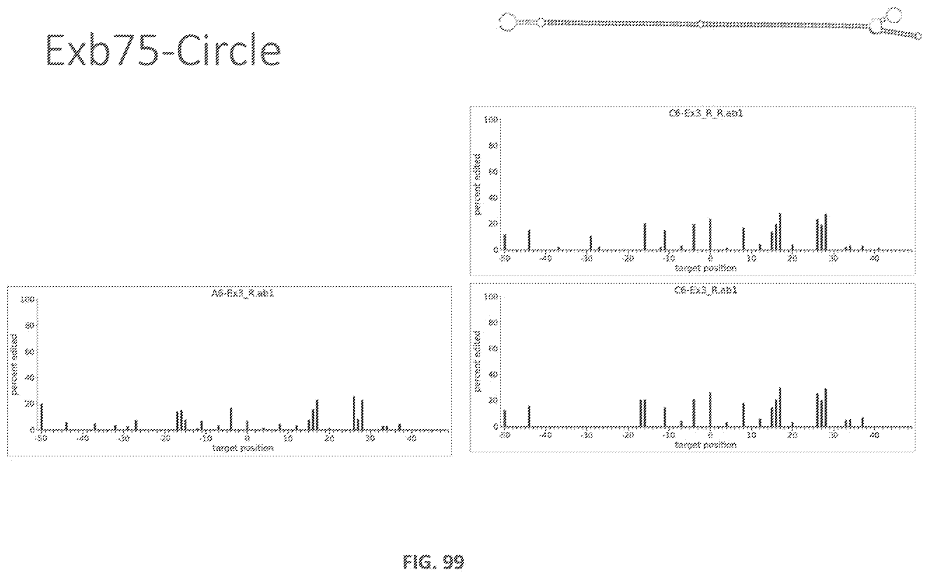

FIG. 99-FIG. 103 show plots of RNA editing at the target A to be edited (“0” on the x-axis) and at RNA editing at off-target positions (represented as black bars at positions that are not “0”). Biological replicates are shown in each column.

Sequences of guides shown in FIG. 99-FIG. 103 are described below in TABLE 23. TABLE 23 below also describes features formed upon hybridization of a given guide to a target RNA, the percent on-target RNA editing observed, on-target editing as a percentage of total RNA editing that is on-target RNA editing, and on-target editing as a percentage of RNA editing at the target in the start site and downstream of the start site in the coding region.

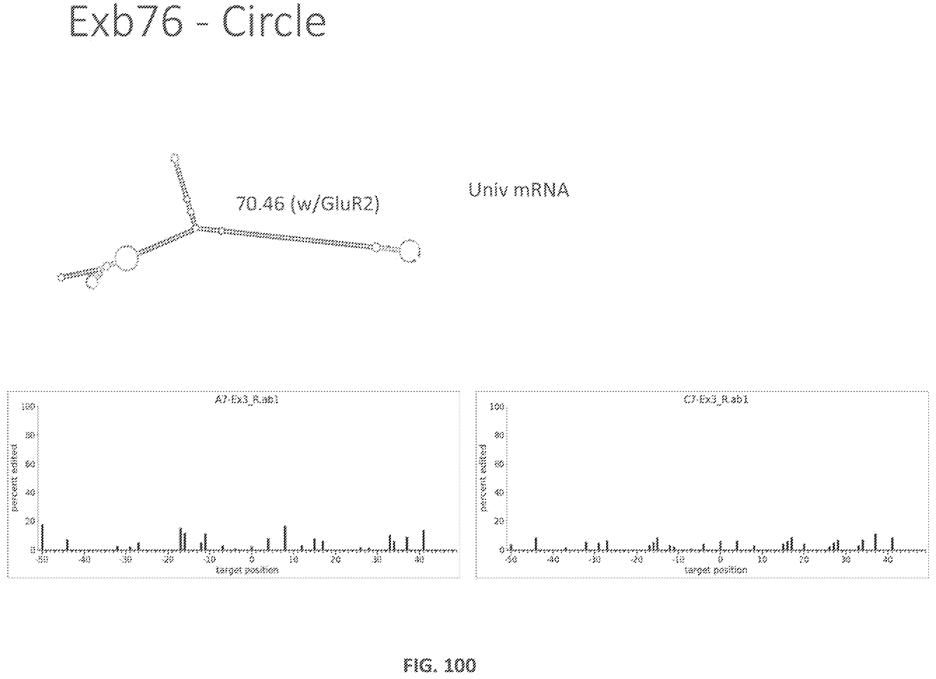

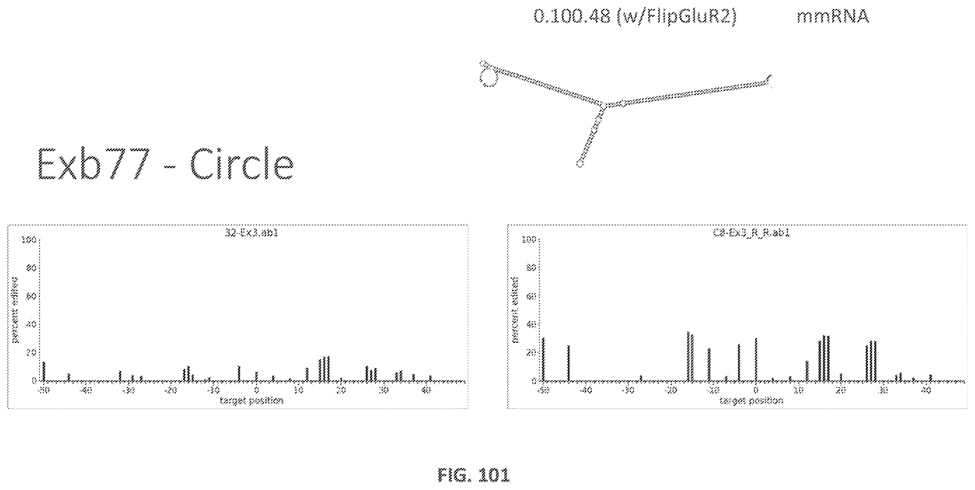

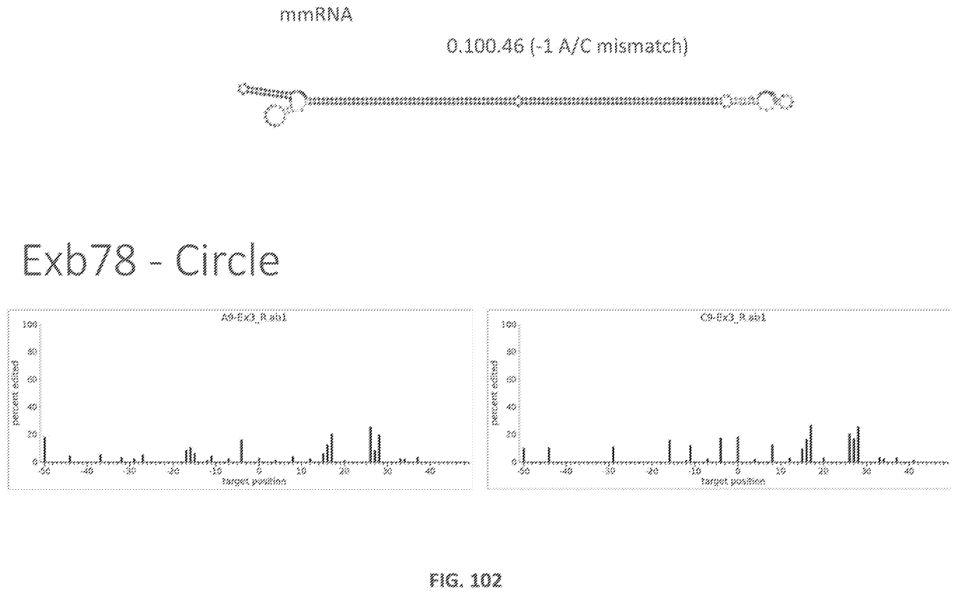

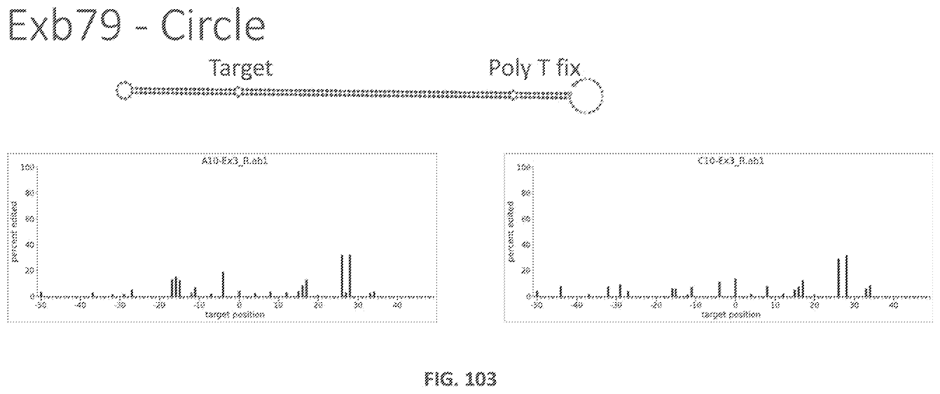

TABLE 23
Guide RNA Sequences

| | | | Percent On- |
|---|---|---|---|
| | | | Target |
| | | | Editing |
| | | | [ADAR1, |
| | | Structural | ADAR1 + |
| SEQ ID NO | Sequence | Features | ADAR2] |
| Target | | — | — |
| Sequenc | | | |
| FIG. 99 | TAGGGATAGGGATAGGGACAACTCCCTC | 3x hnRNP. 100 nt | [3, 22] |
| (SEQ ID NO: | CTTGGCCTTTGAAAGTCCTTTCATGAAT | antisense to target | |
| 104) | ACATCCACGGCTAATGAATTCCTTTACA | w/ 1/1 symmetric | |
| | CCACACTGTCGTCGAATGGCCACTCCC | bulge nt 50 | |
| | AGTTCTC | | |
| FIG. 100 | TAGGGATAGGGATAGGGACAACTCCCTC | 3x hnRNP. 115 nt | [2, 5] |
| (SEQ ID NO: | CTTGGCCTTTGAAAGTCCTTTCATGAAT | antisense to target, | |
| 105) | ACATCCACGGCTAAT [symbol] | w/ 1/1 symmetric | |
| | [symbol] [symbol] | bulge nt 46 and | |
| | [symbol] GAATTCCTTTACACCACA | GluR2 hairpin 54- | |
| | | 98 | |
| FIG. 101 | CAACTCCCTCCTTGGCCTTTGAAAGTC | 145 nt antisense to | [6, 30] |
| (SEQ ID NO: | CTTTCATGAATA [symbol] | target, w/ 1/1 | |
| 106) | [symbol] [symbol] C | symmetric bulge @ | |
| | ATCCACGGCTAATGAATTCCTTTACACC | nt 94 and flip | |
| | ACACTGTCGTCGAATGGCCACTCCCAG | GluR2 hairpin@ nt | |
| | TTCTCA | 42-84 | |
| FIG. 102 | TAGGGATAGGGATAGGGACAACTCCCTC | 3x hnRNP, 100 nt | [2, 18] |
| (SEQ ID NO: | CTTGGCCTTTGAAAGTCCTTTCATGAAT | antisense to target | |
| 107) | ACATCCATAGCTAATGAATTCCTTTACA | w/ 1/1 symmetric | |
| | CCACACTGTCGTCGAATGGCCACTCCC | bulge nt 47 | |
| | AGTTCTCA | | |
| FIG. 103 | TAGGGATAGGGATAGGGACCCTGTTTGG | 3x hnRNP, 100 nt | [4, 13] |
| (SEQ ID NO: | TTCTCTCAGCAGCAGCCACAACTCCCT | antisense to target | |
| 108) | CCTTGGCCTTTGAAAGTCCTTTCATGAA | w/ 1/1 symmetric | |
| | TACATCCATAGCTAATGAATTCCTTTAC | bulge nt 75 | |
| | ACCACA | | |

### Example 15

### Guide RNAs Comprising Targeting LRRK2 mRNA

This example describes LRRK2 editing with guide RNAs of the present disclosure. Self-annealing RNA structures comprising the guide RNA sequences of TABLE 25 and the sequences of the regions targeted by the engineered guide RNAs were contacted with an RNA editing entity (e.g., a recombinant ADAR1 and/or ADAR2) under conditions that allow for the editing of the regions targeted by the guide RNAs. The regions targeted by the guide RNAs were subsequently assessed for editing using next generation sequencing (NGS).

FIG. 212 shows heat maps and structures for exemplary engineered guide RNA sequences targeting a LRRK2 mRNA. The heat map provides visualization of the editing profile at the 10 minute time point. 5 engineered guide RNAs for on-target editing (with no-2 filter) are in the left graph and 5 engineered guide RNAs for on-target editing with minimal-2 editing are depicted on the right graph. The corresponding predicted secondary and tertiary structures are below the heat maps.

Exemplary full guide sequences corresponding to FIG. 212 are shown in TABLE 24.

TABLE 24
Exemplary Engineered Polynucleotide Sequences Targeting LRRK2
mRNA

| SEQ | ID | Full Giude Sequence |
|---|---|---|
| ID NO: | | |
| 145 | LRRK2_guide04_v2 | GCTAAATCGATTCTAAAGCAGGACGGGGTGGTGCC |
| | | GTAGTGTCGTATCTTTGCACTTACAGTC |
| 146 | LRRK2_guide04_v3 | GCTAAATCGATTCTAAAGCAGGACTGGGTGGTGCC |
| | | GTAGTGTCGTATCTTTGCACTTACAGTC |
| 147 | LRRK2_guide04_v1 | GCTAAATCGATTCTAAAGCAGGACCGGGTGGTGCC |
| | | GTAGTGTCGTATCTTTGCACTTACAGTC |
| 148 | LRRK2_guide04_v51 | GCTAAATCGATTCTAGAGCAGGACTGGGTGGTGCC |
| | | GTAGTGTCGTATCTTTGCACTTACAGTC |
| 149 | LRRK2_guide04_v27 | GCTAAATCGATTCTACAGCAGGACTGGGTGGTGCC |
| | | GTAGTGTCGTATCTTTGCACTTACAGTC |
| 150 | LRRK2_guide03_v15 | GCTAAATCGATTCTAAAGCATGACTGGGTGGTGCC |
| | | GTAGTCAGCAATCTTTGCACTTACAGTC |
| 151 | LRRK2_guide03_v3 | GCTAAATCGATTCTAAAGCAGGACTGGGTGGTGCC |
| | | GTAGTCAGCAATCTTTGCACTTACAGTC |
| 152 | LRRK2_guide03_v9 | GCTAAATCGATTCTAAAGCAGTACTGGGTGGTGCC |
| | | GTAGTCAGCAATCTTTGCACTTACAGTC |
| 153 | LRRK2 guide03_v63 | GCTAAATCGATTCTAGAGCATGACTGGGTGGTGCC |
| | | GTAGTCAGCAATCTTTGCACTTACAGTC |
| 154 | LRRK2_guide03_v50 | GCTAAATCGATTCTAGAGCAGGACGGGGTGGTGCC |
| | | GTAGTCAGCAATCTTTGCACTTACAGTC |

TABLE 25 below depicts the resulting editing for each guide. Each Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 25
Engineered Polynucleotide Sequences Targeting LRRK2 mRNA, 45 mers
for In Cis-Editing Experiments

| SEQ | | | | |
|---|---|---|---|---|
| ID | | Guide | | |
| NO | ID | Sequence | Structural Features (target/guide) | Metrics |
| SEQ | LRRK2_ | ATTCTAAAGCAGG | 5/5 symmetric internal loop at −6 | ADAR2 on target: 85.80% |
| ID | guide04_ | ACGGGGTGGTGCC | position (UGCUG-GUCGU) | ADAR2 specificity: 1.1 |
| NO: | v2 | GTAGTGTCGTATC | 1/1 A/C mismatch at 0 position 1/1 U/G | |
| 396 | | TTTGCA | wobble base pair at position +4 | |
| | | | 1/1 U/G wobble base pair at position +5 | |
| | | | 1/1 G/U wobble base pair at position +6 | |
| | | | 1/1 U/G wobble base pair at position +8 | |
| | | | 1/1 A/G mismatch at +10 position | |
| | | | 1/1 A/G mismatch at +13 position | |
| | | | 1/1 G/A mismatch at +19 position | |
| SEQ | LRRK2_ | ATTCTAAAGCAGG | 5/5 symmetric internal loop at −6 | ADAR2 on target: 87.17% |
| ID | guide04_ | ACTGGGTGGTGCC | position (UGCUG-GUCGU) | ADAR2 specificity: 1.09 |
| NO: | v3 | GTAGTGTCGTATC | 1/1 A/C mismatch at 0 position 1/1 U/G | |
| 397 | | TTTGCA | wobble base pair at position +4 | |
| | | | 1/1 U/G wobble base pair at position +5 | |
| | | | 1/1 G/U wobble base pair at position +6 | |
| | | | 1/1 U/G wobble base pair at position +8 | |
| | | | 1/1 A/G mismatch at +13 position | |
| | | | 1/1 G/A mismatch at +19 position | |
| SEQ | LRRK2_ | ATTCTAAAGCAGG | 5/5 symmetric internal loop at −6 | ADAR2 on target: 79.38% |
| ID | guide04_ | ACCGGGTGGTGCC | position (UGCUG-GUCGU) | ADAR2 specificity: 1.05 |
| NO: | v1 | GTAGTGTCGTATC | 1/1 A/C mismatch at 0 position 1/1 U/G | |
| 398 | | TTTGCA | wobble base pair at position +4 | |
| | | | 1/1 U/G wobble base pair at position +5 | |
| | | | 1/1 G/U wobble base pair at position +6 | |
| | | | 1/1 U/G wobble base pair at position +8 | |
| | | | 1/1 A/C mismatch at +10 position | |
| | | | 1/1 A/G mismatch at +13 position | |
| | | | 1/1 G/A mismatch at +19 position | |
| SEQ | LRRK2_ | ATTCTAGAGCAGG | 5/5 symmetric internal loop at −6 | ADAR2 on target: 78.91% |
| ID | guide04_ | ACTGGGTGGTGCC | position (UGCUG-GUCGU) | ADAR2 specificity: 1.07 |
| NO: | v51 | GTAGTGTCGTATC | 1/1 A/C mismatch at 0 position 1/1 U/G | |
| 399 | | TTTGCA | wobble base pair at position +4 | |
| | | | 1/1 U/G wobble base pair at position +5 | |
| | | | 1/1 G/U wobble base pair at position +6 | |
| | | | 1/1 U/G wobble base pair at position +8 | |
| | | | 1/1 A/G mismatch at +13 position | |
| | | | 1/1 G/G mismatch at +19 position | |
| SEQ | LRRK2_ | ATTCTACAGCAGG | 5/5 symmetric internal loop at −6 | ADAR2 on target: 79.32% |
| ID | guide04_ | ACTGGGTGGTGCC | position (UGCUG-GUCGU) | ADAR2 specificity: 1.08 |
| NO: | v27 | GTAGTGTCGTATC | 1/1 A/C mismatch at 0 position 1/1 U/G | |
| 400 | | TTTGCA | wobble base pair at position +4 | |
| | | | 1/1 U/G wobble base pair at position +5 | |
| | | | 1/1 G/U wobble base pair at position +6 | |
| | | | 1/1 U/G wobble base pair at position +8 | |
| | | | 1/1 A/G mismatch at +13 position | |
| SEQ | LRRK2_ | ATTCTAAAGCATG | 1/1 A/C mismatch at 0 position 1/1 U/G | ADAR2 on target: 74.97% |
| ID | guide03_ | ACTGGGTGGTGCC | wobble base pair at position +4 | ADAR2 specificity: 0.71 |
| NO: | v15 | GTAGTCAGCAATC | 1/1 U/G wobble base pair at position +5 | |
| 401 | | TTTGCA | 1/1 G/U wobble base pair at position +6 | |
| | | | 1/1 U/G wobble base pair at position +8 | |
| | | | 2/2 symmetric bulge at +13 position | |
| | | | (AC-UG) | |
| | | | 1/1 G/A mismatch at +19 position | |
| SEQ | LRRK2_ | ATTCTAAAGCAGG | 1/1 A/C mismatch at 0 position 1/1 U/G | ADAR2 on target: 74.86% |
| ID | guide03_ | ACTGGGTGGTGCC | wobble base pair at position +4 | ADAR2 specificity: 0.73 |
| NO: | v3 | GTAGTCAGCAATC | 1/1 U/G wobble base pair at position +5 | |
| 402 | | TTTGCA | 1/1 G/U wobble base pair at position +6 | |
| | | | 1/1 U/G wobble base pair at position | |
| | | | +8 | |
| | | | 1/1 A/G mismatch at +13 position | |
| | | | 1/1 G/A mismatch at +19 position | |
| SEQ | LRRK2_ | ATTCTAAAGCAGT | 1/1 A/C mismatch at 0 position 1/1 U/G | ADAR2 on target: 71.85% |
| ID | guide03_ | ACTGGGTGGTGCC | wobble base pair at position +4 | ADAR2 specificity: 0.56 |
| NO: | v9 | GTAGTCAGCAATC | 1/1 U/G wobble base pair at position +5 | |
| 403 | | TTTGCA | 1/1 G/U wobble base pair at position +6 | |
| | | | 1/1 U/G wobble base pair at position | |
| | | | +8 | |
| | | | 1/1 G/A mismatch at +19 position | |
| SEQ | LRRK2_ | ATTCTAGAGCATG | 1/1 A/C mismatch at 0 position 1/1 U/G | ADAR2 on target: 66.25% |
| ID | guide03_ | ACTGGGTGGTGCC | wobble base pair at position +4 | ADAR2 specificity: 0.81 |
| NO: | v63 | GTAGTCAGCAATC | 1/1 U/G wobble base pair at position +5 | |
| 404 | | TTTGCA | 1/1 G/U wobble base pair at position +6 | |
| | | | 1/1 U/G wobble base pair at position | |
| | | | +8 | |
| | | | 2/2 symmetric bulge at +13 position | |
| | | | (AC-UG) | |
| | | | 1/1 G/G mismatch at +19 position | |
| SEQ | LRRK2_ | ATTCTAGAGCAGG | 1/1 A/C mismatch at 0 position 1/1 U/G | ADAR2 on target: 69.87% |
| ID | guide03_ | ACGGGGTGGTGCC | wobble base pair at position +4 | ADAR2 specificity: 0.82 |
| NO: | v50 | GTAGTCAGCAATC | 1/1 U/G wobble base pair at position +5 | |
| 405 | | TTTGCA | 1/1 G/U wobble base pair at position +6 | |
| | | | 1/1 U/G wobble base pair at position | |
| | | | +8 | |
| | | | 1/1 A/G mismatch at +10 position | |
| | | | 1/1 A/G mismatch at +13 position | |
| | | | 1/1 G/G mismatch at +19 position | |

### Example 16

### Engineered Guide RNAs with a Dumbbell Design and Targeting LRRK2 mRNA

This example describes engineered guide RNA comprising a dumbbell design and targeting LRRK2 mRNA. A dumbbell design in an engineered guide RNA comprises two symmetrical internal loops, wherein the target A to be edited is positioned between the two symmetrical loops for selective editing of the target A. The two symmetrical internal loops are each formed by 6 nucleotides on the guide RNA side of the dsRNA substrate and 6 nucleotides on the target RNA side of the dsRNA substrate. In this example, the target A is an A at position 6055 of a LRRK2 mRNA, which encodes for a pathogenic G2019S mutant protein. Exemplary engineered guide RNAs with a dumbbell design and targeting LRRK2 mRNA are shown in TABLE 26 and FIG. 213.

TABLE 26
Exemplary Dumbbell Engineered Guide RNA Sequences

| SEQ | | | | |
|---|---|---|---|---|
| ID | | | Structural Features | |
| NO | ID | Sequence | (target/guide) | Metrics |
| SEQ | 871 113.57 | CCCTGGTGTGCCCTCTGAT | 6/6 symmetric internal loop | Target A editing: |
| ID | | GTTTTTTAGGGGATTCTAC | 7/7 symmetric internal loop | ADAR1 = 20%, |
| NO: | | AGTAGGACTGAGCACTGC | 1/1 TU/C mismatch | ADAR1/ADAR2 = |
| 155 | | CGAGCTGGGCAATCTTTG | 1 A/G mismatch | 25% |
| | | CATACTACGCAGCATTGG | 6/6 symmetric internal loop | −2 A editing: |
| | | GATACAGTGTGAAAAGCA | | ADAR1 = 2%, |
| | | GCACCCTGGTGTGCCCTCT | | ADAR1/ADAR2 = |
| | | GATGTTTTTTAGGGGATTC | | 2% |
| | | TACAGTAGGACTGAGCAC | | |
| | | TGCCGAGCTGGGCAATCT | | |
| | | TTGCATACTACGCAGCATT | | |
| | | GGGATACAGTGTGAAAAG | | |
| | | CAGCAgtgg | | |
| | | AATTTTTGGAG | | |
| | | CAGGTTTTCTGACTTCGGT | | |
| | | CGGAAAACCCCT | | |
| SEQ | 860 113.57 | CCCTGGTGTGCCCTCTGAT | 6/6 symmetric internal loop | Target A editing: |
| ID | | GTTTTTTAGGGGATTCTAC | 1 C/C mismatch | ADAR1 = 20%, |
| NO: | | AGCAGTACAGAGGACTGC | 3/3 symmetric bulge | ADAR1/ADAR2 = |
| 156 | | CGAGGTCACCAATCTTTGC | 3/3 symmetric bulge | 32% |
| | | ATACTACGCAGCATTGGG | 1 A/A mismatch | −2 A editing: |
| | | ATACAGTGTGAAAAGCAG | 6/6 symmetric internal loop | ADAR1 = 1%, |
| | | CACCCTGGTGTGCCCTCTG | | ADAR1/ADAR2 = |
| | | ATGTTTTTTAGGGGATTCT | | 5% |
| | | ACAGCAGTACAGAGGACT | | |
| | | GCCGAGGTCACCAATCTTT | | |
| | | GCATACTACGCAGCATTG | | |
| | | GGATACAGTGTGAAAAGC | | |
| | | AGCAgtggAATTTTTGGAG | | |
| | | CAGGTTTTCTGACTTCGGT | | |
| | | CGGAAAACCCCT | | |
| SEQ | 1976 113.57 | CCCTGGTGTGCCCTCTGAT | 6/6 symmetric internal loop | Target A editing: |
| ID | | GTTTTTTAGGGGATTCTAC | 1 C/A mismatch | ADAR1 = 30%, |
| NO: | | AACAGTACTGAGCTATCC | 7/7 symmetric internal loop | ADAR1/ADAR2 = |
| 157 | | CGAATTCAACAATCTTTGC | 1 T/TU/U mismatch | 40% |
| | | ATACTACGCAGCATTGGG | 1 C/A mismatch | −2 A editing: |
| | | ATACAGTGTGAAAAGCAG | 6/6 symmetric internal loop | ADAR1 = 6%, |
| | | CACCCTGGTGTGCCCTCTG | | ADAR1/ADAR2 = |
| | | ATGTTTTTTAGGGGATTCT | | 10% |
| | | ACAACAGTACTGAGCTAT | | |
| | | CCCGAATTCAACAATCTTT | | |
| | | GCATACTACGCAGCATTG | | |
| | | GGATACAGTGTGAAAAGC | | |
| | | AGCAgtggAATTTTTGGAG | | |
| | | CAGGTTTTCTGACTTCGGT | | |
| | | CGGAAAACCCCT | | |
| SEQ | 919 113.57 | CCCTGGTGTGCCCTCTGAT | 6/6 symmetric internal loop | Target A editing: |
| ID | | GTTTTTTAGGGGCTTCTAC | 5/5 symmetric internal loop | ADAR1 = 21%, |
| NO: | | AGCAGTTCGGAGGAATCC | 1 G/G mismatch | ADAR1/ADAR2 = |
| 158 | | CGAGGTCAGCAATCTTTG | 3/3 symmetric bulge | 30% |
| | | CATACTACGCAGCATTGG | 7/7 symmetric internal loop | −2 A editing: |
| | | GATACAGTGTGAAAAGCA | | ADAR1 = 1%, |
| | | GCACCCTGGTGTGCCCTCT | | ADAR1/ADAR2 = |
| | | GATGTTTTTTAGGGGCTTC | | 5% |
| | | TACAGCAGTTCGGAGGAA | | |
| | | TCCCGAGGTCAGCAATCTT | | |
| | | TGCATACTACGCAGCATT | | |
| | | GGGATACAGTGTGAAAAG | | |
| | | CAGCAgtgg | | |
| | | AATTTTTGGAG | | |
| | | CAGGTTTTCTGACTTCGGT | | |
| | | CGGAAAACCCCT | | |
| SEQ | 2108 113.57 | CCCTGGTGTGCCCTCTGAT | 6/6 symmetric internal loop | Target A editing: |
| ID | | GTTTTTTAGGGGATTCTAC | 3/3 symmetric bulge | ADAR1 = 22%, |
| NO: | | GGCGGTACTGACCAATCC | 1 C/C mismatch | ADAR1/ADAR2 = |
| 159 | | CGTAGTTAGCAATCTTTGC | 6/6 symmetric internal loop | 35% |
| | | ATACTACGCAGCATTGGG | | −2 A editing: |
| | | ATACAGTGTGAAAAGCAG | | ADAR1 = 5%, |
| | | CCCCTGGTGTGCCCTCTGA | | ADAR1/ADAR2 = |
| | | TGTTTTTTAGGGGATTCTA | | 20% |
| | | CGGCGGTACTGACCAATC | | |
| | | CCGTAGTTAGCAATCTTTG | | |
| | | CATACTACGCAGCATTGG | | |
| | | GATACAGTGTGAAAAGCA | | |
| | | GCgtggAATTTTTGGAG | | |
| | | CAGGTTTTCTGACTTCGGT | | |
| | | CGGAAAACCCCT | | |
| SEQ | 1700 113.57 | CCCTGGTGTGCCCTCTGAT | 6/6 symmetric internal loop | Target A editing: |
| ID | | GTTTTTTAGGGGATTCTAC | 10/10 symmetric internal loop | ADAR1 = 20%, |
| NO: | | CGCAGTACTACCCGATCC | 1 TU/C mismatch | ADAR1/ADAR2 = |
| 160 | | CGTAGTCAGCAATCTTTGC | 6/6 symmetric internal loop | 30% |
| | | ATACTACGCAGCATTGGG | | −2 A editing: |
| | | ATACAGTGTGAAAAGCAG | | ADAR1 = 3%, |
| | | CACCCTGGTGTGCCCTCTG | | ADAR1/ADAR2 = |
| | | ATGTTTTTTAGGGGATTCT | | 11% |
| | | ACCGCAGTACTACCCGAT | | |
| | | CCCGTAGTCAGCAATCTTT | | |
| | | GCATACTACGCAGCATTG | | |
| | | GGATACAGTGTGAAAAGC | | |
| | | AGCAgtggAATTTTTGGAG | | |
| | | CAGGTTTTCTGACTTCGGT | | |
| | | CGGAAAACCCCT | | |
| SEQ | 844 113.57 | CCCTGGTGTGCCCTCTGAT | 6/6 symmetric internal loop | Target A editing: |
| ID | | GTTTTTTAGGGGATTCTAC | 1 C/A mismatch | ADAR1 = 20%, |
| NO: | | GGCAGTTCATAGCAATCC | 3/3 symmetric bulge | ADAR1/ADAR2 = |
| 161 | | CGTAGTCAACAATCTTTGC | 4/4 symmetric bulge | 25% |
| | | CTACTACGCAGCATTGGG | 6/6 symmetric internal loop | −2 A editing: |
| | | ATACAGTGTGAAAAGCAG | | ADAR1 = 5%, |
| | | CACCCTGGTGTGCCCTCTG | | ADAR1/ADAR2 = |
| | | ATGTTTTTTAGGGGATTCT | | 22% |
| | | ACGGCAGTTCATAGCAAT | | |
| | | CCCGTAGTCAACAATCTTT | | |
| | | GCCTACTACGCAGCATTG | | |
| | | GGATACAGTGTGAAAAGC | | |
| | | AGCAgtggAATTTTTGGAG | | |
| | | CAGGTTTTCTGACTTCGGT | | |
| | | CGGAAAACCCCT | | |

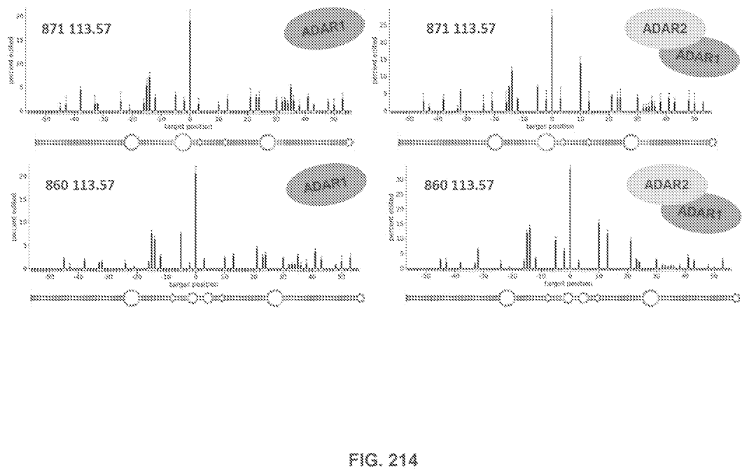

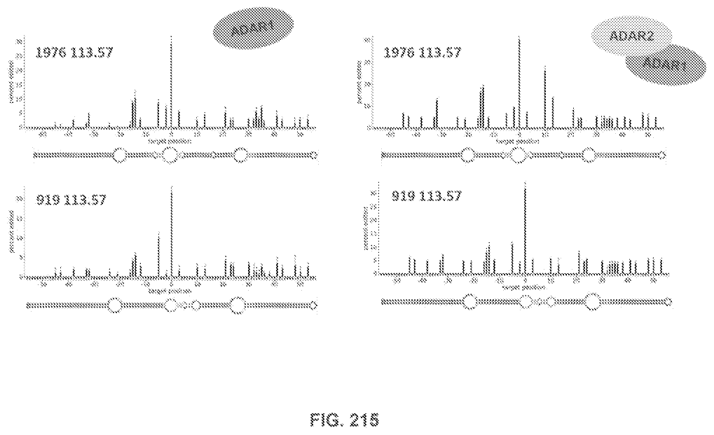

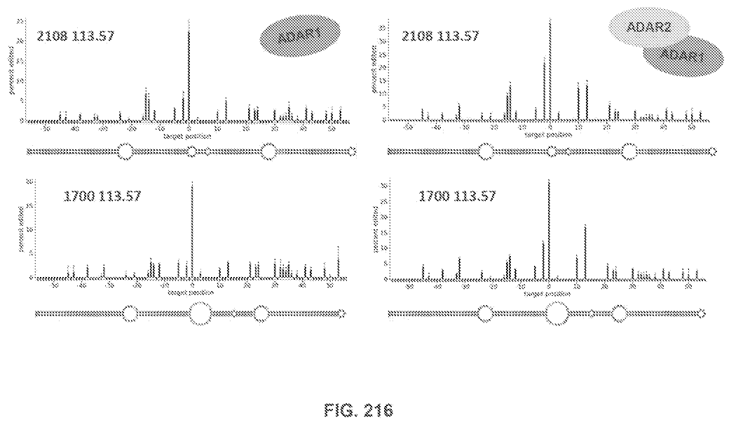

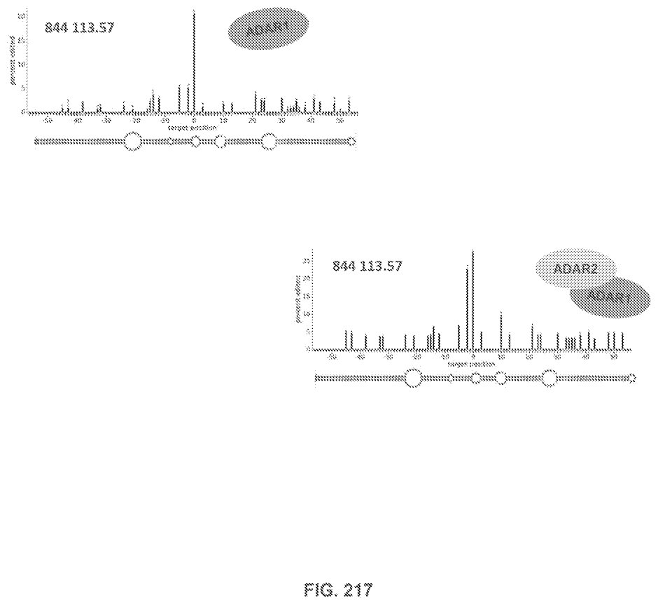

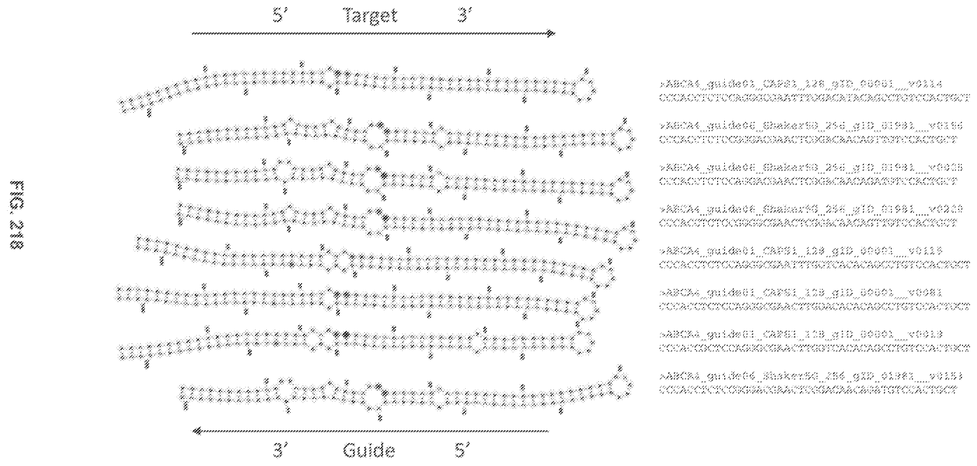

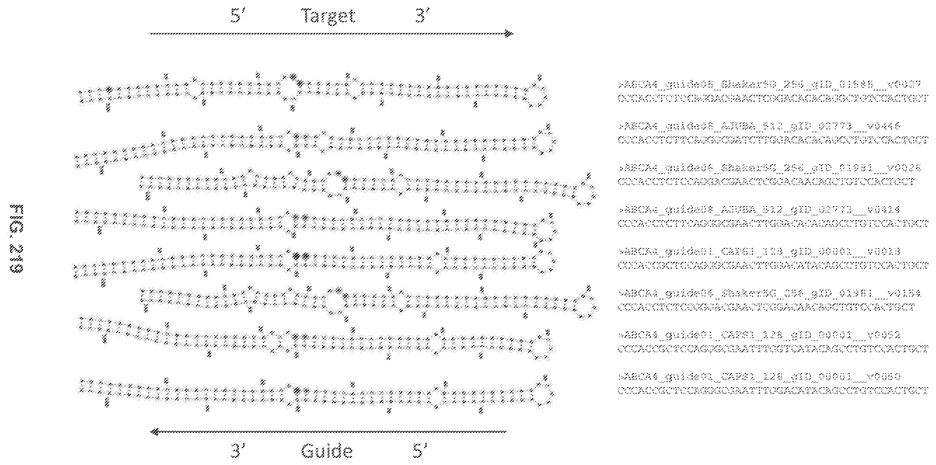

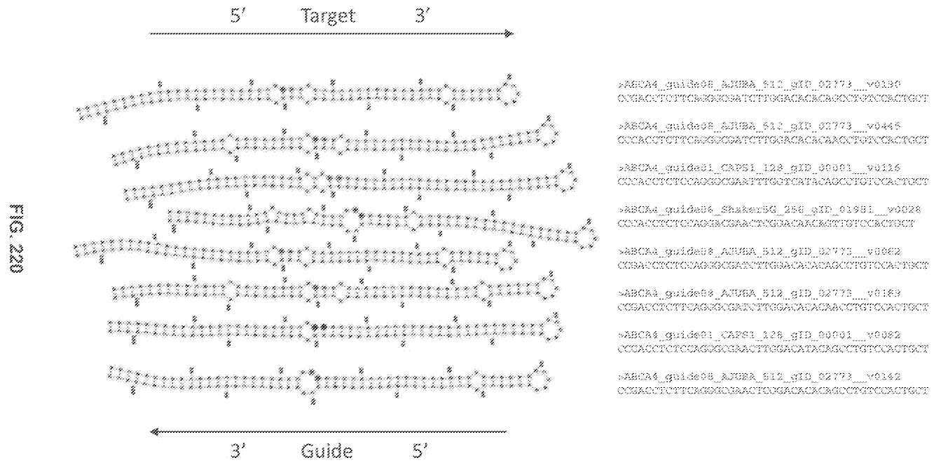

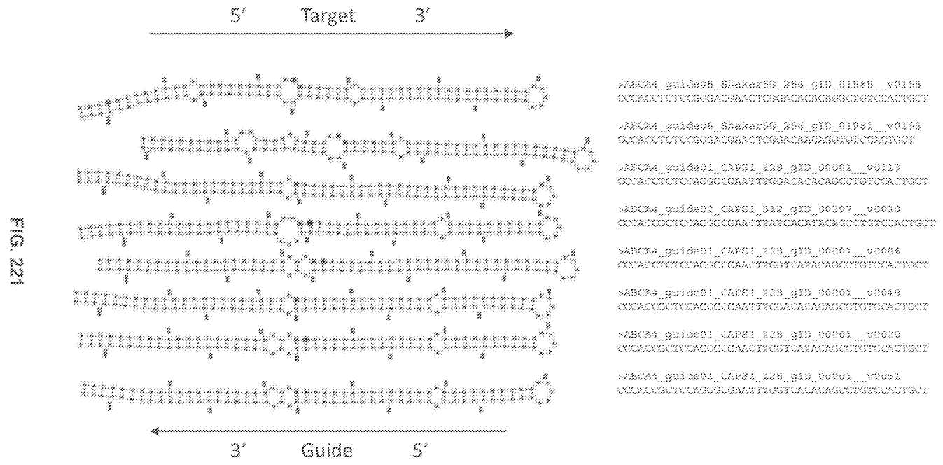

FIG. 214-FIG. 217 shows graphs of on-target and off-target ADAR1 editing and ADAR1+ADAR2 editing of the nucleotide of the codon encoding the G2019S mutation for exemplary dumbbell guide RNA sequences (see TABLE 26). For FIG. 214-FIG. 217, 750 ng of plasmid was transfected in 20,000 cells. HEK293 cells naturally expressing ADAR1 were transfected with a piggyBac vector carrying the LRRK2 minigene or were transfected with a piggyBac vector carrying the LRRK2 minigene and ADAR2. Engineered guide RNAs were administered to cells and RNA editing was quantified 48 hours post transfection.

### Example 17

### Engineered Guide RNAs Targeting LRRK2 mRNA

This example describes guide RNAs that target LRRK2 mRNA. Self-annealing RNA structures comprising the guide RNA sequences of TABLE 27 and the sequences of the regions targeted by the guide RNAs were contacted with an RNA editing entity (e.g., a recombinant ADAR1 and/or ADAR2) for 30 minutes under conditions that allow for the editing of the regions targeted by the guide RNAs. The regions targeted by the guide RNAs were subsequently assessed for editing using next generation sequencing (NGS). The guide RNAs of TABLE 27 showed specific editing of the A nucleotide at position 6055 of the mRNA encoding the LRRK2 G2019S. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 27
Exemplary Guide RNAs that target LRRK2 mRNA

| SEQ | | | | |
|---|---|---|---|---|
| ID | | | Structural Features | |
| NO | ID | Sequence | (target/guide) | Metrics |
| SEQ | LRRK2_ | ATTCTACAGCACGA | 1/1 C/U mismatch at −4 position | ADAR1 on target: 55.03% |
| ID | bnP_CR_ | CTGAGCAATGCCGT | 1/1 A/C mismatch at 0 position | ADAR2 on target: 89.21% |
| NO: | ID3796_ | ATTCAGCAATCTTTG | 2/2 symmetric bulge at +13 | ADAR1 specificity: 0.55 |
| 162 | count1909 | CA | position (AC-CG) | ADAR2 specificity: 1.12 |
| SEQ | LRRK2_ | ATTCTAGAGCAGTA | 1/1 C/C mismatch at −8 position | ADAR1 on target: 91.69% |
| ID | bnPCR_ | CTGAGCAATGCCGT | 1/1 G/U wobble base pair at −6 | ADAR2 on target: 89.35% |
| NO: | ID47662_ | GGTTACCAATCTTTG | position | ADAR1 specificity: 0.61 |
| 163 | count362 | CA | 1/1 U/G wobble base pair at −3 | ADAR2 specificity: 1.04 |
| | | | position | |
| | | | 1/1 A/C mismatch at 0 position | |
| | | | 1/1 G/G mismatch at +19 | |
| | | | position | |
| SEQ | LRRK2_ | ATTCTACAGCAGTA | 1/1 C/A mismatch at −17 | ADAR1 on target: 72.09% |
| ID | bnPCR_ | GTAAGCTTTGCCGA | position | ADAR2 on target: 89.77% |
| NO: | ID117434_ | AGTGAGCAATCTTTA | 1/1 G/G mismatch at -6 position | ADAR1 specificity: 0.75 |
| 164 | count761 | CA | 1/1 A/A mismatch at -2 position | ADAR2 specificity: 0.92 |
| | | | 1/1 A/C mismatch at 0 position | |
| | | | 2/2 symmetric bulge at +4 | |
| | | | position (UU-UU) | |
| | | | 1/1 C/A mismatch at +9 position | |
| | | | 1/1 G/G mismatch at +11 | |
| | | | position | |
| SEQ | LRRK2_ | ATTCTACAGCACGA | 1/1 U/G wobble base pair at −10 | ADAR1 on target: 69.21% |
| ID | bnPCR_ | CTGAGCAAGGCCGT | position | ADAR2 on target: 90.00% |
| NO: | ID70875_ | AGTCAGCGATCTTTG | 1/1 A/C mismatch at 0 position | ADAR1 specificity: 0.54 |
| 165 | count310 | CA | 1/1 A/G mismatch at +3 position | ADAR2 specificity: 1.32 |
| | | | 2/2 symmetric bulge at +13 | |
| | | | position (AC-CG) | |
| SEQ | LRRK2_ | ATTCTACAGCACTAC | 1/1 U/U mismatch at −7 position | ADAR1 on target: 29.21% |
| ID | bnPCR_ | GGGGCAATGCCGAA | 1/1 A/A mismatch at −2 position | ADAR2 on target: 90.13% |
| NO: | ID26374_ | GTCTGCAATCTTTGC | 1/1 A/C mismatch at 0 position | ADAR1 specificity: 0.46 |
| 166 | count337 | A | 1/1 U/G wobble base pair at +8 | ADAR2 specificity: 1.01 |
| | | | position | |
| | | | 1/1 A/G mismatch at +10 | |
| | | | position | |
| | | | 1/1 C/C mismatch at +14 | |
| | | | position | |
| SEQ | LRRK2_ | ATTCTACGGCGGTAC | 1/1 G/U wobble base pair at −6 | ADAR1 on target: 93.44% |
| ID | bnPCR_ | TGACCAATCCCGTA | position | ADAR2 on target: 90.18% |
| NO: | ID34601_ | GTTAGCAATCTTTGC | 1/1 A/C mismatch at 0 position | ADAR1 specificity: 0.91 |
| 167 | count2108 | A | 1/1 C/C mismatch at +2 position | ADAR2 specificity: 1.32 |
| | | | 1/1 C/C mismatch at +7 position | |
| | | | 1/1 U/G wobble base pair at +15 | |
| | | | position | |
| | | | 1/1 U/G wobble base pair at +18 | |
| | | | position | |
| SEQ | LRRK2_ | ATTCTACAGCGGTAC | 1/1 G/U wobble base pair at −13 | ADAR1 on target: 74.58% |
| ID | bnPCR_ | TCATCAATGCCGTAG | position | ADAR2 on target: 90.33% |
| NO: | ID8689_ | TCGTCAATTTTTGCA | 1/1 C/U mismatch at −8 position | ADAR1 specificity: 0.49 |
| 168 | count926 | | 1/1 U/G wobble base pair at −7 | ADAR2 specificity: 1.02 |
| | | | position | |
| | | | 1/1 A/C mismatch at 0 position | |
| | | | 3/3 symmetric bulge at +7 | |
| | | | position (CUC-CAU) | |
| | | | 1/1 U/G wobble base pair at +15 | |
| | | | position | |
| SEQ | LRRK2_ | ATTCTAAAGCCGTAC | 2/2 symmetric bulge at −7 | ADAR1 on target: 33.31% |
| ID | bnPCR _ | TAAGCAGTACCGTA | position (CU-CC) | ADAR2 on target: 90.37% |
| NO: | ID4669_ | GTCCCCAATCTTTGC | 1/1 A/C mismatch at 0 position | ADAR1 specificity: 0.47 |
| 169 | count1929 | A | 1/1 C/A mismatch at +2 position | ADAR2 specificity: 0.81 |
| | | | 1/1 U/G wobble base pair at +4 | |
| | | | position | |
| | | | 1/1 C/A mismatch at +9 position | |
| | | | 1/1 U/C mismatch at +15 | |
| | | | position | |
| | | | 1/1 G/A mismatch at +19 | |
| | | | position | |
| SEQ | LRRK2_ | ATTCTAGAGCCATAC | 1/1 A/A mismatch at −2 position | ADAR1 on target: 13.05% |
| ID | bnPCR_ | TGTTCAATGCCGAA | 1/1 A/C mismatch at 0 position | ADAR2 on target: 90.41% |
| NO: | ID24531_ | GTCAGCAATCTTTGC | 2/2 symmetric bulge at +7 | ADAR1 specificity: 0.69 |
| 170 | count2149 | A | position (CU-UU) | ADAR2 specificity: 1.31 |
| | | | 2/2 symmetric bulge at +14 | |
| | | | position (CU-CA) | |
| | | | 1/1 G/G mismatch at +19 | |
| | | | position | |
| SEQ | LRRK2_ | ATTCTACAGCGGTAC | 1/1 U/U mismatch at −7 position | ADAR1 on target: 79.59% |
| ID | bnPCR_ | TGGGCAATGCCGTG | 1/1 U/G wobble base pair at −3 | ADAR2 on target: 90.46% |
| NO: | ID38909_ | GTCTGCAATCTTTGC | position | ADAR1 specificity: 0.54 |
| 171 | count2105 | A | 1/1 A/C mismatch at 0 position | ADAR2 specificity: 1.16 |
| | | | 1/1 U/G wobble base pair at +8 | |
| | | | position | |
| | | | 1/1 U/G wobble base pair at +15 | |
| | | | position | |
| SEQ | LRRK2_ | ATTCTACAGCTGTAC | 1/1 C/C mismatch at −8 position | ADAR1 on target: 92.87% |
| ID | bnPCR_ | TGAGGAATGCCGTC | 1/1 G/U wobble base pair at −6 | ADAR2 on target: 90.75% |
| NO: | ID13205_ | TTTACCAATCTTTGC | position | ADAR1 specificity: 0.63 |
| 172 | count2438 | A | 2/2 symmetric bulge at −3 | ADAR2 specificity: 1.36 |
| | | | position (CU-CU) | |
| | | | 1/1 A/C mismatch at 0 position | |
| | | | 1/1 G/G mismatch at +6 position | |
| | | | 1/1 U/U mismatch at +15 | |
| | | | position | |
| SEQ | LRRK2_ | ATTCTACAGCAGTAC | 1/1 G/U wobble base pair at −6 | ADAR1 on target: 88.30% |
| ID | bnPCR_ | TTATCAGTGCCGTTG | position | ADAR2 on target: 91.15% |
| NO: | ID74600_ | TTAGCAATCTTTGCA | 1/1 U/U mismatch at −3 position | ADAR1 specificity: 0.59 |
| 173 | count480 | | 1/1 A/C mismatch at 0 position | ADAR2 specificity: 1.2 |
| | | | 1/1 U/G wobble base pair at +4 | |
| | | | position | |
| | | | 3/3 symmetric bulge at +7 | |
| | | | position (CUC-UAU) | |
| SEQ | LRRK2_ | ATTCTATAGCTGGAC | 1/1 C/U mismatch at −8 position | ADAR1 on target: 26.20% |
| ID | bnPCR_ | TGAGCTCTGCCGTAG | 1/1 U/G wobble base pair at −7 | ADAR2 on target: 91.42% |
| NO: | ID11052_ | TCGTCAATCTTTGCA | position | ADAR1 specificity: 0.74 |
| 174 | count1602 | | 1/1 A/C mismatch at 0 position | ADAR2 specificity: 1.23 |
| | | | 2/2 symmetric bulge at +4 | |
| | | | position (UU-UC) | |
| | | | 3/3 symmetric bulge at +13 | |
| | | | position (ACU-UGG) | |
| | | | 1/1 G/U wobble base pair at +19 | |
| | | | position | |
| SEQ | LRRK2_ | ATTCTACAGCAATAC | 1/1 C/C mismatch at −8 position | ADAR1 on target: 81.04% |
| ID | bnPCR_ | TCAGCAGTGCCGTA | 1/1 A/C mismatch at 0 position | ADAR2 on target: 91.91% |
| NO: | ID22281_ | GTCACCAATCTTTGC | 1/1 U/G wobble base pair at +4 | ADAR1 specificity: 0.64 |
| 175 | count2661 | A | position | ADAR2 specificity: 1.07 |
| | | | 1/1 C/C mismatch at +9 position | |
| | | | 1/1 C/A mismatch at +14 | |
| | | | position | |
| SEQ | LRRK2_ | ATTCTACAACAGTAC | 1/1 U/U mismatch at −7 position | ADAR1 on target: 92.15% |
| ID | bnPCR_ | TGCGCACCGCCGTA | 1/1 A/C mismatch at 0 position | ADAR2 on target: 92.65% |
| NO: | ID20990_ | GTCTGCAATCTTTGC | 2/2 symmetric bulge at +3 | ADAR1 specificity: 0.68 |
| 176 | count3129 | A | position (AU-CC) | ADAR2 specificity: 1.05 |
| | | | 1/1 U/C mismatch at +8 position | |
| | | | 1/1 C/A mismatch at +17 | |
| | | | position | |
| SEQ | LRRK2_ | ATTCTAGAGCCGTAC | 1/1 C/U mismatch at −8 position | ADAR1 on target: 14.68% |
| ID | bnPCR_ | TGCGCTTTGCCGTAG | 1/1 A/C mismatch at 0 position | ADAR2 on target: 92.90% |
| NO: | ID27527_ | TCATCAATCTTTGCA | 2/2 symmetric bulge at +4 | ADAR1 specificity: 0.54 |
| 177 | count488 | | position (UU-UU) | ADAR2 specificity: 1.15 |
| | | | 1/1 U/C mismatch at +8 position | |
| | | | 1/1 U/C mismatch at +15 | |
| | | | position | |
| | | | 1/1 G/G mismatch at +19 | |
| | | | position | |
| SEQ | LRRK2_ | ATTCTACAGCCGTAC | 3/3 symmetric bulge at −6 | ADAR1 on target: 62.87% |
| ID | CbnPR_ | TGCCCAATGCCGAA | position (CUG-AAC) | ADAR2 on target: 93.88% |
| NO: | ID18596_ | GTAACCAATCTTTGC | 1/1 A/A mismatch at −2 position | ADAR1 specificity: 0.6 |
| 178 | count3437 | A | 1/1 A/C mismatch at 0 position | ADAR2 specificity: 1.14 |
| | | | 2/2 symmetric bulge at +7 | |
| | | | position (CU-CC) | |
| | | | 1/1 U/C mismatch at +15 | |
| | | | position | |
| SEQ | LRRK2_ | ATTCTACCGTGGGAC | 1/1 U/U mismatch at −7 position | ADAR1 on target: 84.30% |
| ID | bnPCR_ | TGAGCAGTCCCGTA | 1/1 A/C mismatch at 0 position | ADAR2 on target: 94.10% |
| NO: | ID5048_ | GTCTGCAATCTTTGC | 1/1 C/C mismatch at +2 position | ADAR1 specificity: 1.02 |
| 179 | count2719 | A | 1/1 U/G wobble base pair at +4 | ADAR2 specificity: 1.29 |
| | | | position | |
| | | | 1/1 A/G mismatch at +13 | |
| | | | position | |
| | | | 1/1 U/G wobble base pair at +15 | |
| | | | position | |
| | | | 1/1 G/U wobble base pair at +16 | |
| | | | position | |
| | | | 1/1 U/C mismatch at +18 | |
| | | | position | |
| SEQ | LRRK2_ | ATTCTACAGCAGTAC | 1/1 G/A mismatch at −1 position | ADAR1 on target: 16.06% |
| ID | bnPCR_ | TGTTCAGTGCGCTAG | 2/2 symmetric bulge at −1 | ADAR2 on target: 94.39% |
| NO: | ID3292_ | TAAGCAATCTTTGCA | position spanning 0 position | ADAR1 specificity: 0.84 |
| 180 | count1951 | | (CA-GC) | ADAR2 specificity: 1.29 |
| | | | 1/1 U/G wobble base pair at +4 | |
| | | | position | |
| | | | 2/2 symmetric bulge at +7 | |
| | | | position (CU-UU) | |
| SEQ | LRRK2_ | ATTCTACAGGTGTAC | 1/1 U/C mismatch at −10 | ADAR1 on target: 71.92% |
| ID | bnPCR_ | TGTTCTATGCCGTTG | position | ADAR2 on target: 94.50% |
| NO: | ID92487_ | TTAGCCATCTTTGCA | 1/1 G/U wobble base pair at −6 | ADAR1 specificity: 0.72 |
| 181 | count914 | | position | ADAR2 specificity: 1.26 |
| | | | 1/1 U/U mismatch at −3 position | |
| | | | 1/1 A/C mismatch at 0 position | |
| | | | 1/1 U/U mismatch at +5 position | |
| | | | 2/2 symmetric bulge at +7 | |
| | | | position (CU-UU) | |
| | | | 2/2 symmetric bulge at +15 | |
| | | | position (UG-GU) | |
| SEQ | LRRK2_ | ATTCTAGGGCAGTA | 4/4 symmetric bulge at −3 | ADAR1 on target: 88.68% |
| ID | bnPCR_ | CTGAGAGATGCCGA | position spanning 0 position | ADAR2 on target: 30.88% |
| NO: | ID12934_ | TGTCAGCAATCTTTG | (UACA-CGAU) | ADAR1 specificity: 1.21 |
| 182 | count1814 | CA | 1/1 U/G wobble base pair at +5 | ADAR2 specificity: 0.64 |
| | | | position | |
| | | | 1/1 G/A mismatch at +6 position | |
| | | | 1/1 U/G wobble base pair at +18 | |
| | | | position | |
| | | | 1/1 G/G mismatch at +19 | |
| | | | position | |
| SEQ | LRRK2_ | ATTCTACAGCAGTAT | 1/1 A/C mismatch at 0 position | ADAR1 on target: 88.73% |
| ID | bnPCR_ | TGGGCAGTCCCGTA | 1/1 C/C mismatch at +2 position | ADAR2 on target: 78.61% |
| NO: | ID22125_ | GTCAGCAATCTTTGC | 1/1 U/G wobble base pair at +4 | ADAR1 specificity: 1.06 |
| 183 | count837 | A | position | ADAR2 specificity: 1.19 |
| | | | 1/1 U/G wobble base pair at +8 | |
| | | | position | |
| | | | 1/1 G/U wobble base pair at +11 | |
| | | | position | |
| SEQ | LRRK2_ | ATTCTACAGCAGGA | 1/1 C/U mismatch at −6 position | ADAR1 on target: 89.12% |
| ID | bnPCR_ | CTGGGCGATCCCGT | 1/1 A/C mismatch at 0 position | ADAR2 on target: 79.41% |
| NO: | ID31607_ | AGTCATCAATCTTTG | 1/1 C/C mismatch at +2 position | ADAR1 specificity: 1.08 |
| 184 | count1105 | CA | 1/1 U/G wobble base pair at +5 | ADAR2 specificity: 0.96 |
| | | | position | |
| | | | 1/1 U/G wobble base pair at +8 | |
| | | | position | |
| | | | 1/1 A/G mismatch at +13 | |
| | | | position | |
| SEQ | LRRK2_ | ATTCTACAGCAGTAC | 1/1 C/C mismatch at −8 position | ADAR1 on target: 89.96% |
| ID | bnPCR_ | TAAGCTATGGCGTA | 2/2 symmetric bulge at −4 | ADAR2 on target: 6.89% |
| NO: | ID17685_ | CGCACCAATCTTTGC | position (AC-CG) | ADAR1 specificity: 0.98 |
| 185 | count2957 | A | 2/2 symmetric bulge at 0 | ADAR2 specificity: 0.34 |
| | | | position (AG-GC) | |
| | | | 1/1 U/U mismatch at +5 position | |
| | | | 1/1 C/A mismatch at +9 position | |
| SEQ | LRRK2_ | ATTCTACAGTAGGA | 1/1 U/G wobble base pair at −7 | ADAR1 on target: 90.20% |
| ID | bnPCR_ | CTGAGCACTGCCGA | position | ADAR2 on target: 88.24% |
| NO: | ID29209_ | GCTGGGCAATCTTTG | 1/1 G/G mismatch at −6 position | ADAR1 specificity: 1.33 |
| 186 | count871 | CA | 0/1 asymmetric bulge at −4 | ADAR2 specificity: 1.07 |
| | | | position (-C) | |
| | | | 1/0 asymmetric bulge at −2 | |
| | | | position (C-) | |
| | | | 1/1 A/C mismatch at 0 position | |
| | | | 1/1 U/C mismatch at +4 position | |
| | | | 1/1 A/G mismatch at +13 | |
| | | | position | |
| | | | 1/1 G/U wobble base pair at +16 | |
| | | | position | |
| SEQ | LRRK2_ | ATTCTACCGCAGTAC | 3/3 symmetric bulge at −6 | ADAR1 on target: 90.49% |
| ID | bnPCR_ | TGAGCAAAGCCGAG | position (CUG-AAA) | ADAR2 on target: 78.40% |
| NO: | ID33976_ | GTAAACAATCTTTGC | 1/1 U/G wobble base pair at −3 | ADAR1 specificity: 1.02 |
| 187 | count681 | A | position | ADAR2 specificity: 0.74 |
| | | | 1/1 A/A mismatch at −2 position | |
| | | | 1/1 A/C mismatch at 0 position | |
| | | | 1/1 A/A mismatch at +3 position | |
| | | | 1/1 U/C mismatch at +18 | |
| | | | position | |
| SEQ | LRRK2_ | ATTCTACAGCAGTAC | 1/1 U/C mismatch at −7 position | ADAR1 on target: 90.61% |
| ID | CbnPR_ | TGAGCAATTCCGTTA | 3/3 symmetric bulge at −3 | ADAR2 on target: 52.23% |
| NO: | ID39201_ | ACCGCAATCTTTGCA | position (ACU-UAA) | ADAR1 specificity: 1.05 |
| 188 | count637 | | 1/1 A/C mismatch at 0 position | ADAR2 specificity: 0.69 |
| | | | 1/1 C/U mismatch at +2 position | |
| SEQ | LRRK2_ | ATTCTACAGTAGTCC | 1/1 U/C mismatch at −7 position | ADAR1 on target: 90.66% |
| ID | bnPCR_ | GGAGCTATTCCGTA | 1/1 A/C mismatch at 0 position | ADAR2 on target: 27.30% |
| NO: | ID87993_ | GTCCGCAATCTTTGC | 1/1 C/U mismatch at +2 position | ADAR1 specificity: 1.04 |
| 189 | count219 | A | 1/1 U/U mismatch at +5 position | ADAR2 specificity: 0.45 |
| | | | 1/1 A/G mismatch at +10 | |
| | | | position | |
| | | | 1/1 U/C mismatch at +12 | |
| | | | position | |
| | | | 1/1 G/U wobble base pair at +16 | |
| | | | position | |
| SEQ | LRRK2_ | ATTCTACAGCAGGA | 1/1 U/G wobble base pair at −7 | ADAR1 on target: 90.80% |
| ID | bnPCR_ | CTGAGCAATGGAGT | position | ADAR2 on target: 23.12% |
| NO: | ID31517_ | AGTCGGCAATCTTTG | 2/2 symmetric bulge at 0 | ADAR1 specificity: 1.01 |
| 190 | count1083 | CA | position (AG-GA) | ADAR2 specificity: 0.47 |
| | | | 1/1 A/G mismatch at +13 | |
| | | | position | |
| SEQ | LRRK2_ | ATTCTACCGCAGTAC | 1/1 A/C mismatch at 0 position | ADAR1 on target: 90.83% |
| ID | bnPCR_ | TACCCGATCCCGTAG | 1/1 C/C mismatch at +2 position | ADAR2 on target: 35.42% |
| NO: | ID28134 | TCAGCAATCTTTGCA | 1/1 U/G wobble base pair at +5 | ADAR1 specificity: 1.14 |
| 191 | count1700 | | position | ADAR2 specificity: 0.81 |
| | | | 3/3 symmetric bulge at +7 | |
| | | | position (CUC-ACC) | |
| | | | 1/1 U/C mismatch at +18 | |
| | | | position | |
| SEQ | LRRK2_ | ATTCTACAGGAGTTC | 1/1 C/U mismatch at −8 position | ADAR1 on target: 91.01% |
| ID | bnPCR_ | TGAGCATTCCCGTCG | 1/1 U/C mismatch at −3 position | ADAR2 on target: 39.14% |
| NO: | ID38170_ | TCATCAATCTTTGCA | 1/1 A/C mismatch at 0 position | ADAR1 specificity: 1.2 |
| 192 | count773 | | 3/3 symmetric bulge at +2 | ADAR2 specificity: 0.69 |
| | | | position (CAU-UUC) | |
| | | | 1/1 U/U mismatch at +12 | |
| | | | position | |
| | | | 1/1 G/G mismatch at +16 | |
| | | | position | |
| SEQ | LRRK2_ | ATTCTACAGCAGTAC | 1/1 C/C mismatch at −8 position | ADAR1 on target: 91.49% |
| ID | bnPCR_ | AGAGGACTGCCGAG | 1/1 U/G wobble base pair at −3 | ADAR2 on target: 81.85% |
| NO: | ID33405_ | GTCACCAATCTTTGC | position | ADAR1 specificity: 1.03 |
| 193 | count860 | A | 1/1 A/A mismatch at −2 position | ADAR2 specificity: 0.85 |
| | | | 1/1 A/C mismatch at 0 position | |
| | | | 1/1 U/C mismatch at +4 position | |
| | | | 1/1 G/G mismatch at +6 position | |
| | | | 1/1 A/A mismatch at +10 | |
| | | | position | |
| SEQ | LRRK2_ | ATTCTACAGAAGGA | 1/1 A/C mismatch at 0 position | ADAR1 on target: 91.67% |
| ID | bnPCR_ | CCGAGCAGTCCCGT | 1/1 C/C mismatch at +2 position | ADAR2 on target: 89.02% |
| NO: | ID39919_ | AGTCAGCAATCTTTG | 1/1 U/G wobble base pair at +4 | ADAR1 specificity: 0.73 |
| 194 | count1633 | CA | position | ADAR2 specificity: 0.8 |
| | | | 1/1 A/C mismatch at +10 | |
| | | | position | |
| | | | 1/1 A/G mismatch at +13 | |
| | | | position | |
| | | | 1/1 G/A mismatch at +16 | |
| | | | position | |
| SEQ | LRRK2_ | ATTCTACAGCATTAC | 3/3 symmetric bulge at −4 | ADAR1 on target: 92.10% |
| ID | bnPCR_ | TGAGCAATCCCGTAT | position (GAC-UUA) | ADAR2 on target: 92.36% |
| NO: | ID21333_ | TAAGCAATCTTTGCA | 1/1 A/C mismatch at 0 position | ADAR1 specificity: 1.03 |
| 195 | count1914 | | 1/1 C/C mismatch at +2 position | ADAR2 specificity: 0.7 |
| | | | 1/1 C/U mismatch at +14 | |
| | | | position | |
| SEQ | LRRK2_ | ATTCTACAGCAGTAC | 1/1 G/U wobble base pair at −6 | ADAR1 on target: 92.46% |
| ID | CbnPR_ | GAAGCAATCCCGCC | position | ADAR2 on target: 20.91% |
| NO: | ID97672_ | GTTAGCAATCTTTGC | 2/2 symmetric bulge at −2 | ADAR1 specificity: 1.01 |
| 196 | count397 | A | position (UA-CC) | ADAR2 specificity: 0.54 |
| | | | 1/1 A/C mismatch at 0 position | |
| | | | 1/1 C/C mismatch at +2 position | |
| | | | 2/2 symmetric bulge at +9 | |
| | | | position (CA-GA) | |
| SEQ | LRRK2_ | ATTCTACAGCAGTAC | 1/1 G/A mismatch at −6 position | ADAR1 on target: 93.75% |
| ID | bnPCR_ | TGTTCAATTCCGATG | 6/6 symmetric internal loop at | ADAR2 on target: 56.09% |
| NO: | ID36558_ | TAAGCAATCTTTGCA | −3 position spanning 0 position | ADAR1 specificity: 1.4 |
| 197 | count1785 | | (UACAGC-UCCGAU) | ADAR2 specificity: 0.92 |
| | | | 2/2 symmetric bulge at +7 | |
| | | | position (CU-UU) | |
| SEQ | LRRK2_ | ATTCTACAACAGTAC | 1/1 C/A mismatch at −8 position | ADAR1 on target: 94.19% |
| ID | bnPCR_ | TGAGCTATCCCGAAT | 10/10 symmetric internal loop at | ADAR2 on target: 90.41% |
| NO: | ID16690_ | TCAACAATCTTTGCA | −4 position spanning 0 position | ADAR1 specificity: 1.01 |
| 198 | count1976 | | (CUACAGCAUU- | ADAR2 specificity: 0.85 |
| | | | UAUCCCGAAU) | |
| | | | 1/1 C/A mismatch at +17 | |
| | | | position | |
| SEQ | LRRK2_ | ATTCTACGGCAGTTC | 1/1 C/A mismatch at −8 position | ADAR1 on target: 94.28% |
| ID | bnPCR_ | ATAGCAATCCCGTA | 1/1 A/C mismatch at 0 position | ADAR2 on target: 12.67% |
| NO: | ID22357_ | GTCAACAATCTTTGC | 1/1 C/C mismatch at +2 position | ADAR1 specificity: 1.15 |
| 199 | count844 | C | 2/2 symmetric bulge at +9 | ADAR2 specificity: 0.71 |
| | | | position (CA-AU) | |
| | | | 1/1 U/U mismatch at +12 | |
| | | | position | |
| | | | 1/1 U/G wobble base pair at +18 | |
| | | | position | |
| SEQ | LRRK2_ | CTTCTACAGCAGTTC | 1/1 U/G wobble base pair at −3 | ADAR1 on target: 94.31% |
| ID | bnPCR_ | GGAGGAATCCCGAG | position | ADAR2 on target: 74.70% |
| NO: | ID8030_ | GTCAGCAATCTTTGC | 1/1 A/1 mismatch at −2 position | ADAR1 specificity: 1.36 |
| 200 | count919 | A | 1/1 A/C mismatch at 0 position | ADAR2 specificity: 0.86 |
| | | | 1/1 C/C mismatch at +2 position | |
| | | | 1/1 G/G mismatch at +6 position | |
| | | | 3/3 symmetric bulge at +10 | |
| | | | position (AGU-UCG) | |
| | | | 5/0 asymmetric internal loop at | |
| | | | +24 position (AUAUA-) | |
| | | | 1/1 G/U wobble base pair at +29 | |
| | | | position | |
| SEQ | LRRK2_ | ATTCTACAGCTGTAC | 1/1 U/C mismatch at −7 position | ADAR1 on target: 95.93% |
| ID | bnPCR_ | TAGGCTATCCCGTAG | 1/1 A/C mismatch at 0 position | ADAR2 on target: 82.09% |
| NO: | ID43647_ | TCCGCAATCTTTGCA | 1/1 C/C mismatch at +2 position | ADAR1 specificity: 1.11 |
| 201 | count763 | | 1/1 U/U mismatch at +5 position | ADAR2 specificity: 0.96 |
| | | | 1/1 U/G wobble base pair at +8 | |
| | | | position | |
| | | | 1/1 C/A mismatch at +9 position | |
| | | | 1/1 U/U mismatch at +15 | |
| | | | position | |
| SEQ | LRRK2_ | ATTCTACAGCCGTAC | 1/1 U/U mismatch at −7 position | ADAR1 on target: 90.48% |
| ID | bnPCR_ | TGGGAAATCCCGTA | 1/1 A/C mismatch at 0 position | ADAR2 on target: 87.02% |
| NO: | ID4389_ | GTCTGCAATCTTTGC | 1/1 C/C mismatch at +2 position | ADAR1 specificity: 0.94 |
| 202 | count1135 | A | 1/1 G/A mismatch at +6 position | ADAR2 specificity: 1.14 |
| | | | 1/1 U/G wobble base pair at +8 | |
| | | | position | |
| | | | 1/1 U/C mismatch at +15 | |
| | | | position | |
| SEQ | LRRK2_ | ATTCTACAGAAGTA | 1/1 A/C mismatch at −16 | ADAR1 on target: 84.41% |
| ID | bnPCR_ | CAGAGACATCCCGT | position | ADAR2 on target: 82.65% |
| NO: | ID38794_ | AGTTAACAATCTTCG | 1/1 C/A mismatch at −8 position | ADAR1 specificity: 1.09 |
| 203 | count776 | CA | 1/1 G/U wobble base pair at −6 | ADAR2 specificity: 1.18 |
| | | | position | |
| | | | 5/4 asymmetric internal loop at 0 | |
| | | | position (AGCAU-UCCC) | |
| | | | 0/1 asymmetric bulge at +6 | |
| | | | position (-A) | |
| | | | 1/1 A/A mismatch at +10 | |
| | | | position | |
| | | | 1/1 G/A mismatch at +16 | |
| | | | position | |
| SEQ | LRRK2_ | ATTCTACAGCCGTAC | 1/1 U/C mismatch at −7 position | ADAR1 on target: 87.33% |
| ID | bnPCR_ | TGAGCTATCCCGTAG | 1/1 A/C mismatch at 0 position | ADAR2 on target: 88.42% |
| NO: | ID37802_ | TCCGCAATCTTTGCA | 1/1 C/C mismatch at +2 position | ADAR1 specificity: 0.97 |
| 204 | count2433 | | 1/1 U/U mismatch at +5 position | ADAR2 specificity: 1.06 |
| | | | 1/1 U/C mismatch at +15 | |
| | | | position | |
| SEQ | LRRK2_ | ATTCTACTGCAGCAC | 2/2 symmetric bulge at −7 | ADAR1 on target: 93.09% |
| ID | bnPCR_ | CGAGCAATCCCGCA | position (CU-CC) | ADAR2 on target: 84.08% |
| NO: | ID489_ | TTCCCCAATCTTTGC | 7/7 symmetric internal loop at | ADAR1 specificity: 0.62 |
| 205 | count3336 | A | −4 position spanning 0 position | ADAR2 specificity: 0.78 |
| | | | (CUACAGC-CCCGCAU) | |
| | | | 1/1 A/C mismatch at +10 | |
| | | | position | |
| | | | 1/1 A/C mismatch at +13 | |
| | | | position | |
| | | | 1/1 U/U mismatch at +18 | |
| | | | position | |
| SEQ | LRRK2_ | ATTCTACAGCAGTCC | 1/1 A/C mismatch at 0 position | ADAR1 on target: 85.47% |
| ID | bnPCR_ | TGAGCAGTACCGTA | 1/1 C/A mismatch at +2 position | ADAR2 on target: 84.08% |
| NO: | ID50412_ | GTCAGCAATCTTTGC | 1/1 U/G wobble base pair at +4 | ADAR1 specificity: 0.86 |
| 206 | count1318 | A | position | ADAR2 specificity: 0.96 |
| | | | 1/1 U/C mismatch at +12 | |
| | | | position | |
| SEQ | LRRK2_ | ATTCTAAGGCAGTA | 1/1 U/G wobble base pair at −7 | ADAR1 on target: 85.92% |
| ID | CbnPR_ | CTGAGCCGCGCCGT | position | ADAR2 on target: 82.19% |
| NO: | ID27560_ | AGTCGGCAATCTTTG | 1/1 A/C mismatch at 0 position | ADAR1 specificity: 0.98 |
| 207 | count2218 | CA | 1/1 A/C mismatch at +3 position | ADAR2 specificity: 0.88 |
| | | | 1/1 U/G wobble base pair at +4 | |
| | | | position | |
| | | | 1/1 U/C mismatch at +5 position | |
| | | | 1/1 U/G wobble base pair at +18 | |
| | | | position | |
| | | | 1/1 G/A mismatch at +19 | |
| | | | position | |

### Example 18

### Engineered Guide RNAs Targeting SNCA mRNA

This example describes engineered guide RNAs that target SNCA mRNA. Self-annealing RNA structures comprising the engineered guide RNA sequences of TABLE 28 and the sequences of the regions targeted by the guide RNAs were contacted with an RNA editing entity (e.g., a recombinant ADAR1 and/or ADAR2) under conditions that allow for the editing of the regions targeted by the guide RNAs. The regions targeted by the guide RNAs were subsequently assessed for editing using next generation sequencing (NGS). The guide RNAs of TABLE 28 showed specific editing of the A nucleotide at translation initiation start site (TIS; the A in the ATG start coding with genomic coordinates: hg38 chr4: 89835667 strand −1) of SNCA mRNA. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 28
Exemplary guide RNAs that target SNCA mRNA

| SEQ | | | | |
|---|---|---|---|---|
| ID | | Guide RNA | Structural Features | |
| NO | ID | Sequence | (target/guide) | Metrics |
| SEQ | SNCA_ | GAAAGTACTTTGAT | 1/1 A/C mismatch at 0 position | ADAR2 on target: 95.68% |
| ID | guide01_v7 | GAATACATCCACGG | 1/1 G/G mismatch at +14 position | ADAR2 specificity: 1.95 |
| NO: | | CTAATGAATTCCTT | 1/1 G/A mismatch at +19 position | |
| 208 | | TAC | | |
| SEQ | SNCA_ | GAAAGTCCTTTCAA | 4/4 symmetric bulge at −6 position | ADAR2 on target: 94.39% |
| ID | guide02_v25 | GAATACATCCACGG | (UCAU-UACU) | ADAR2 specificity: 1.94 |
| NO: | | CTATACTATTCCTT | 1/1 A/C mismatch at 0 position | |
| 209 | | TAC | 1/1 A/A mismatch at +12 position | |
| SEQ | SNCA_ | GAAAGTCCTTTGAT | 1/1 A/C mismatch at 0 position | ADAR2 on target: 95.92% |
| ID | guide01_v24 | GCATACATCCACGG | 1/1 U/C mismatch at +10 position | ADAR2 specificity: 1.93 |
| NO: | | CTAATGAATTCCTT | 1/1 G/G mismatch at +14 position | |
| 210 | | TAC | | |
| SEQ | SNCA_ | GAAAGTCCTTTGAT | 4/4 symmetric bulge at −6 position | ADAR2 on target: 94.10% |
| ID | guide02_v24 | GCATACATCCACGG | (UCAU-UACU) | ADAR2 specificity: 1.93 |
| NO: | | CTATACTATTCCTT | 1/1 A/C mismatch at 0 position | |
| 211 | | TAC | 1/1 U/C mismatch at +10 position | |
| | | | 1/1 G/G mismatch at +14 position | |
| SEQ | SNCA_ | GAAAGTCCTTTGAG | 4/4 symmetric bulge at −6 position | ADAR2 on target: 88.96% |
| ID | guide02_v22 | GCATACATCCACGG | (UCAU-UACU) | ADAR2 specificity: 1.88 |
| NO: | | CTATACTATTCCTT | 1/1 A/C mismatch at 0 position | |
| 212 | | TAC | 1/1 U/C mismatch at +10 position | |
| | | | 1/1 A/G mismatch at +12 position | |
| | | | 1/1 G/G mismatch at +14 position | |
| SEQ | SNCA | GAAAGTCCTTTGAG | 4/4 symmetric bulge at −6 position | ADAR2 on target: 93.74% |
| ID | guide02_v21 | GAATACATCCACGG | (UCAU-UACU) | ADAR2 specificity: 1.93 |
| NO: | | CTATACTATTCCTT | 1/1 A/C mismatch at 0 position | |
| 213 | | TAC | 1/1 A/G mismatch at +12 position | |
| | | | 1/1 G/G mismatch at +14 position | |
| SEQ | SNCA_ | GAAAGTCCTTTGAA | 4/4 symmetric bulge at −6 position | ADAR2 on target: 84.65% |
| ID | guide02_v18 | GCATACATCCACGG | (UCAU-UACU) | ADAR2 specificity: 1.84 |
| NO: | | CTATACTATTCCTT | 1/1 A/C mismatch at 0 position | |
| 214 | | TAC | 1/1 U/C mismatch at +10 position | |
| | | | 1/1 A/A mismatch at +12 position | |
| | | | 1/1 G/G mismatch at +14 position | |
| SEQ | SNCA_ | GAAAGTACTTTCAC | 4/4 symmetric bulge at −6 position | ADAR2 on target: 89.43% |
| ID | guide02_v11 | GAATACATCCACGG | (UCAU-UACU) | ADAR2 specificity: 1.88 |
| NO: | | CTATACTATTCCTT | 1/1 A/C mismatch at 0 position | |
| 215 | | TAC | 1/1 A/C mismatch at +12 position | |
| | | | 1/1 G/A mismatch at +19 position | |
| SEQ | SNCA_ | GAAAGTCCTTTCAC | 4/4 symmetric bulge at −6 position | ADAR2 on target: 93.20% |
| ID | guide02_v28 | GCATACATCCACGG | (UCAU-UACU) | ADAR2 specificity: 1.93 |
| NO: | | CTATACTATTCCTT | 1/1 A/C mismatch at 0 position | |
| 216 | | TAC | 1/1 U/C mismatch at +10 position | |
| | | | 1/1 A/C mismatch at +12 position | |
| SEQ | SNCA_ | GAAAGTCCTTTCAA | 4/4 symmetric bulge at −6 position | ADAR2 on target: 90.62% |
| ID | guide02_v26 | GCATACATCCACGG | (UCAU-UACU) | ADAR2 specificity: 1.91 |
| NO: | | CTATACTATTCCTT | 1/1 A/C mismatch at 0 position | |
| 217 | | TAC | 1/1 U/C mismatch at +10 position | |
| | | | 1/1 A/A mismatch at +12 position | |

### Example 19

### In Vitro Editing of LRRK2 mRNA in iPSC-Derived LRRK2-G2019S Dopaminergic Neurons

This example describes editing in vitro in induced pluripotent stem cell (iPSC)-derived neurons that can express LRRK2-G2019S mutant protein. Culturing, induction, maturation, and transfection or transduction of iPSC neurons are optimized for screening of editing facilitated by guide RNAs. Each dopaminergic neuronal phenotype is characterized and validated via TaqMan qPCR, flow cytometry, and immunofluorescence. iPSCs, neural stem cells (NSCs), neural progenitor cell (NPCs), or derived neuronal cells are then transfected or transduced with guide RNAs targeting a nucleotide of the codon encoding the LRRK2-G2019S mutation, and editing efficiency is quantified using Sanger sequencing, ddPCR, and amplicon next generation sequencing of the sequence encoding the LRRK2-G2019S locus. In vitro biochemical editing of LRRK2 mRNA that is translated into LRRK2 protein is assessed using LC-MS/MS of the LRRK2 protein, and Western Blot and Meso Scale Discovery (MSD) analysis of LRRK2 substrates (e.g., phospho-Rab (−8,−10,−35), LRRK2 autophosphorylation).

### Example 20

### Ex Vivo Editing of LRRK2 mRNA in Primary Cortical Neurons of hLRRK2-G2019S Mice

This example describes editing of the nucleotide of the codon encoding the LRRK2-G2019S mutation ex vivo in primary cortical neurons isolated from hLRRK2-G2019S mice. Primary microdissection and culturing of the primary neurons from the hLRRK2-G2019S mice are optimized prior to transfection or transduction of the guide RNAs. The isolated cortical neurons are then transfected with guide RNAs targeting the mRNA encoding the LRRK2-G2019S mutation, and editing efficiency is quantified using Sanger sequencing, ddPCR, and amplicon next generation sequencing of the LRRK2-G2019S encoding locus. Further, the ex vivo biochemical editing of LRRK2 mRNA that is translated into LRRK2 protein is assessed using LC-MS/MS of the LRRK2 protein, Western Blot and MSD analysis of LRRK2 substrates (e.g., phospho-Rab (−8,−10,−35), LRRK2 autophosphorylation).

### Example 21

### In Vivo Editing of LRRK2 mRNA in hLRRK2-G2019S Mice

This example describes editing of mRNA encoding LRRK2-G2019S in vivo in hLRRK2-G2019S mice. Guide RNAs targeting mRNA encoding the LRRK2-G2019S mutation are administered to the brain of the hLRRK2-G2019S mice via intracerebroventricular, intraparenchymal, intracisternal, or intrathecal injection. Brain tissue is then isolated from the hLRRK2-G2019S mice and processed to isolate LRRK2 nucleic acid and protein. Editing efficiency is quantified using ddPCR and amplicon next generation sequencing of the locus encoding LRRK2-G2019S. Further, the in vivo biochemical editing of LRRK2 mRNA that is translated into LRRK2 protein is assessed using LC-MS/MS of the LRRK2 protein, Western Blot and MSD analysis of LRRK2 substrates (e.g., phospho-Rab (−8,−10,−35), LRRK2 autophosphorylation).

### Example 22

### In Vitro Editing of SNCA mRNA in LUHMES and iPSC-Derived Dopaminergic Neurons

This example describes SNCA editing in vitro in LUHMES and iPSC-derived dopaminergic neurons. Culturing, induction, differentiation, and transfection and transduction of LUHMES and iPSC-derived neurons are optimized for screening of editing facilitated by guide RNAs. Each dopaminergic neuronal phenotype is characterized and validated via TaqMan qPCR, flow cytometry, and immunofluorescence. Neurons are then transfected or transduced with guide RNAs targeting SNCA, and editing efficiency is quantified using qPCR, ddPCR and amplicon next generation sequencing of the SNCA locus. The in vitro biochemical knockdown of SNCA protein is assessed using Western Blot, ELISA, and MSD analysis of SNCA protein levels.

### Example 23

### Ex Vivo Editing of SNCA mRNA in Primary Cortical Neurons of hSNCA Mice

This example describes SNCA mRNA editing and SNCA protein knockdown ex vivo in primary cortical neurons isolated from hSNCA mice. Primary microdissection and culturing of the primary neurons from the SNCA mice are optimized prior to transfection or transduction of the guide RNAs. The isolated cortical neurons are then transfected or transduced with guide RNAs targeting SNCA, and editing efficiency is quantified using qPCR<ddPCR, and amplicon next generation sequencing of the SNCA locus. Further, the ex vivo biochemical knockdown of SNCA protein is assessed using Western Blot, ELISA, and MSD analysis of SNCA protein levels.

### Example 24

### In Vivo Editing of SNCA mRNA in hSNCA Mice

This example describes SNCA editing in vivo in hSNCA mice. Guide RNAs targeting SNCA are administered to the brain of the hSNCA mice via intracerebroventricular, intraparenchymal, intracisternal, or intrathecal injection. Brain tissue is then isolated from the hSNCA mice and processed to isolate SNCA nucleic acid and protein. Editing efficiency is quantified using qPCR, ddPCR, and amplicon next generation sequencing of the SNCA locus. Further, the in vivo biochemical knockdown of SNCA protein is assessed from the isolated SNCA protein using Western Blot, ELISA, and MSD analysis of SNCA protein levels.

### Example 25

### Engineered Guide RNAs Targeting SERPINA1 mRNA

This example describes engineered guide RNAs that target SERPINA1 mRNA. High throughput screening (HTS) of gRNA sequences against the SERPINA1 E342K mutation identified designs with superior on-target activity and specificity.

Self-annealing RNA structures comprising the engineered guide RNA sequences of TABLE 29 and the sequences of the regions targeted by the guide RNAs were contacted with an RNA editing entity (e.g., a recombinant ADAR1 and/or ADAR2) for 30 minutes under conditions that allow for the editing of the regions targeted by the guide RNAs. The regions targeted by the guide RNAs were subsequently assessed for editing using next generation sequencing (NGS). The guide RNAs of TABLE 29 showed specific editing of the A nucleotide. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 29
Exemplary guide RNAs that target SERPINA1 mRNA

| SEQ | | | | |
|---|---|---|---|---|
| ID | | | | |
| NO | Name | Sequence | Structural Features (target/guide) | Metrics |
| SEQ | SERP_ | GCCCCAGCAGC | 1/1 G/U wobble base pair at −10 position | ADAR1 on target: |
| ID | 006448_ | TTCAGTTCCTTA | 2/0 asymmetric bulge at −8 position (AC-) | 53.40% |
| NO: | SERINA1- | ATCGTCGATGT | 1/1 A/C mismatch at 0 position | ADAR1 specificity: |
| 297 | 20_26_ | AGCACAGCC | 2/2 symmetric bulge at +2 position (GA-AA) | 1.49 |
| | 45.26 | | 1/1 G/U wobble base pair at +8 position | |
| SEQ | SERP_ | ATTCCCATGGC | 1/1 A/C mismatch at 0 position | ADAR1 on target: |
| ID | 006566_ | CCCAGCAGCTT | 2/1 asymmetric bulge at +3 position (AA-A) | 45.30% |
| NO: | SERINA1- | CAGTCCTTACT | 1/1 G/U wobble base pair at +6 position | ADAR1 specificity: |
| 298 | 12_34_ | CGTCGATGGTC | | 1.40 |
| | 45.34 | A | | |
| SEQ | SERP_ | CCTCTAAAAAC | 1/1 A/C mismatch at 0 position | ADAR1 on target: |
| ID | 007612_ | ATGGCTCCCCA | 1/0 asymmetric bulge at +8 position (G-) | 47.50% |
| NO: | RSEINA1- | CAGCTTCAGTC | 2/2 symmetric bulge at +19 position (CU- | ADAR1 specificity: |
| 299 | 6_40_ | CTTTCTCGTCGA | CA) | 1.46 |
| | 45.40 | | 0/1 asymmetric bulge at +23 position (-U) | |
| SEQ | SERP_ | ATGGCCTGTGC | 1/1 U/G wobble base pair at −14 position | ADAR1 on target: |
| ID | 007657_ | AGCTTCAGTCC | 1/1 G/A mismatch at −13 position | 64.20% |
| NO: | SERINA1- | CTTACTCGTCG | 1/1 U/G wobble base pair at −11 position | ADAR1 specificity: |
| 300 | 17_29_ | ATGGCCGTCGG | 0/3 asymmetric bulge at −8 position (-CCG) | 1.49 |
| | 45.29 | AGCTCA | 1/1 A/C mismatch at 0 position | |
| | | | 1/1 A/A mismatch at +3 position | |
| | | | 2/2 symmetric bulge at +20 position (UG- | |
| | | | GU) | |
| | | | 1/1 G/U wobble base pair at +22 position | |
| SEQ | SERP_ | AGTAAAACATG | 1/1 A/C mismatch at 0 position | ADAR1 on target: |
| ID | 010900_ | TACGCCCCAGC | 1/0 asymmetric bulge at +5 position (A-) | 52.60% |
| NO: | SERINA1- | CTTCAGTCCCTT | 2/0 asymmetric bulge at +16 position (CU-) | ADAR1 specificity: |
| 301 | 9_37_ | CTCGTCGA | 0/3 asymmetric bulge at +25 position (-UAC) | 1.52 |
| | 45.37 | | | |
| SEQ | SERP_ | AAAACAATACT | 1/1 A/C mismatch at 0 position | ADAR1 on target: |
| ID | 010980_ | ACCCAGCAGCT | 4/2 asymmetric bulge at +3 position (AAAG- | 49.90% |
| NO: | SERINA1- | TCAGTCCACCT | AC) | ADAR1 specificity: |
| 302 | 12_34_ | CGTCGATGGTC | 4/6 asymmetric internal loop at +24 position | 1.47 |
| | 45.34 | A | (GCCA-AUACUA) | |
| SEQ | SERP_ | CTAATCCATGG | 1/1 A/C mismatch at 0 position | ADAR1 on target: |
| ID | 011202_ | CTACCCCAGCA | 2/0 asymmetric bulge at +11 position (UG-) | 46.10% |
| NO: | SERINA1- | GCTTGTCCCTTT | 0/3 asymmetric bulge at +23 position (-UAC) | ADAR1 specificity: |
| 303 | 9_37_ | CTCGTCGATGG | 3/2 asymmetric bulge at +30 position (UUU- | 1.41 |
| | 45.37 | | UC) | |
| SEQ | SERP_ | TGCTAAAAACA | 1/1 U/C mismatch at -5 position | ADAR1 on target: |
| ID | 011563_ | TGGTGCAGCAG | 1/1 A/C mismatch at 0 position | 42.00% |
| NO: | SERINA1- | CTTCAGTCCTG | 3/2 asymmetric bulge at +3 position (AAA- | ADAR1 specificity: |
| 304 | 9_37_ | ACTCGTCGCTG | GA) | 1.41 |
| | 45.37 | G | 1/1 G/U wobble base pair at +6 position | |
| | | | 2/1 asymmetric bulge at +22 position (GG- | |
| | | | G) | |
| | | | 1/1 G/U wobble base pair at +24 position | |
| SEQ | SERP_ | AAAAACATGGC | 1/1 A/C mismatch at 0 position | ADAR1 on target: |
| ID | 012208_ | CCCAGCAGCTT | 3/3 symmetric bulge at +3 position (AAA- | 46.40% |
| NO: | SERINA1- | CAGTCCCCAAC | CAA) | ADAR1 specificity: |
| 305 | 11_35_ | TCGTCGATGGT | | 1.28 |
| | 45.35 | C | | |
| SEQ | SERP_ | AAAACATGGCC | 1/1 A/C mismatch at 0 position | ADAR1 on target: |
| ID | 013687_ | CCAGCAGCTTC | 2/2 symmetric bulge at +3 position (AA-GA) | 44.90% |
| NO: | SERINA1- | AGTCCCTGACT | | ADAR1 specificity: |
| 306 | 12_34_ | CGTCGATGGTC | | 1.28 |
| | 45.34 | A | | |
| SEQ | SERP_ | CTAAAAACATG | 1/1 C/A mismatch at -1 position | ADAR1 on target: |
| ID | 016311_ | GCACAGCAGCT | 3/3 symmetric bulge at +2 position (GAA- | 47.40% |
| NO: | SERINA1- | TCAGTCCCTCC | CCA) | ADAR1 specificity: |
| 307 | 9_37_ | ATTATCGATGG | 2/1 asymmetric bulge at +22 position (GG-A) | 1.45 |
| | 45.37 | | | |
| SEQ | SERP_ | AACTACAAGGC | 0/1 asymmetric bulge at -7 position (-U) | ADAR1 on target: |
| ID | 016711_ | CCCAGCAGCTT | 1/1 A/C mismatch at 0 position | 48.00% |
| NO: | SERINA1- | CATCCCTTTACT | 2/0 asymmetric bulge at +2 position (-A) | ADAR1 specificity: |
| 308 | 11_35_ | CGTCGATGTGG | 1/0 asymmetric bulge at +10 position (C-) | 1.44 |
| | 45.35 | | 1/1 A/S mismatch at +27 position | |
| SEQ | SERP_ | CTAAAAACATG | 1/0 asymmetric bulge at -6 position (A-) | ADAR1 on target: |
| ID | 017788_ | GCTCCCAGCAG | 0/2 asymmetric bulge at -1 position (-CU) | 51.30% |
| NO: | SERINA1- | CTTCATATCCTT | 1/1 A/C mismatch at 0 position | ADAR1 specificity: |
| 309 | 9_37_ | TCCTGCTTCGA | 1/1 G/U wobble base pair at +8 position | 1.51 |
| | 45.37 | GG | 2/2 symmetric bulge at +9 position (AC-UA) | |
| | | | 0/1 asymmetric bulge at +23 position (-U) | |
| SEQ | SERP_ | TAAAAACATGG | 0/1 asymmetric bulge at -4 position (-A) | ADAR1 on target: |
| ID | 019344_ | CCCCCAGTTCA | 1/1 C/A mismatch at -1 position | 45.90% |
| NO: | SERINA1- | | 0/2 asymmetric bulge at +4 position (-AA) | ADAR1 specificity: |
| 310 | 10_36_ | GTCCCTAATTCT | 1/0 asymmetric bulge at +15 position (G-) | 1.45 |
| | 45.36 | TATCGAATGGT | 2/0 asymmetric bulge at +19 position (CU-) | |
| SEQ | SERP_ | CTAAAAACGCC | 1/1 A/C mismatch at 0 position | ADAR1 on target: |
| ID | 020550_ | CCAGCAGCTTC | 0/3 asymmetric bulge at +5 position (-ACA) | 53.40% |
| NO: | SERINA1- | AGTCCCACATT | 3/0 asymmetric bulge at +26 position (CAU-) | ADAR1 specificity: |
| 311 | 9_37_ | TCTCGTCGATG | | 1.53 |
| | 45.37 | G | | |
| SEQ | SERP_ | GCCCCGTCAGT | 1/1 U/U mismatch at -16 position | ADAR1 on target: |
| ID | 021899_ | TCAGTCCCTGTT | 1/1 G/U wobble base pair at -15 position | 51.50% |
| NO: | SERINA1- | CTCGTCGATGC | 2/2 symmetric bulge at -8 position (AC-CA) | ADAR1 specificity: |
| 312 | 20_26_ | ACAGCATTGCC | 1/1 A/C mismatch at 0 position | 1.48 |
| | 45.26 | | 0/1 asymmetric bulge at +4 position (-G) | |
| | | | 1/0 asymmetric bulge at +15 position (G-) | |
| | | | 1/1 C/U mismatch at +19 position | |
| | | | 1/1 U/G wobble base pair at +20 position | |
| SEQ | SERP_ | CTAAAAACCCA | 1/1 A/C mismatch at 0 position | ADAR1 on target: |
| ID | 024066_ | TGGCGACAGCA | 1/1 A/C mismatch at +5 position | 55.80% |
| NO: | SERINA1- | GCTTCTCCCCTT | 2/0 asymmetric bulge at +10 position (CU-) | ADAR1 specificity: |
| 313 | 9_37_ | CTCGTCGATGG | 2/2 symmetric bulge at +22 position (GG- | 1.49 |
| | 45.37 | | GA | |
| | | | 0/2 asymmetric bulge at +28 position (-CC) | |
| SEQ | SERP_ | AAGCTATGGCC | 0/1 asymmetric bulge at -8 position (-A) | ADAR1 on target: |
| ID | 024794_ | CCAGCAGCTTC | 1/0 asymmetric bulge at -5 position (U-) | 42.30% |
| NO: | SERINA1- | ACCCTTTCTCGT | 1/1 A/C mismatch at 0 position | ADAR1 specificity: |
| 314 | 14_32_ | CGTGGATCC | 2/0 asymmetric bulge at +9 position (AC-) | 1.41 |
| | 45.32 | | 0/1 asymmetric bulge at +28 position (-U) | |
| | | | 1/1 U/G wobble base pair at +30 position | |
| SEQ | SERP_ | AAAACATGGCA | 2/2 symmetric bulge at -6 position (-CA/AC) | ADAR1 on target: |
| ID | 028982_ | CCCCAGCAGCT | 1/1 A/C mismatch at 0 position | 47.00% |
| NO: | SERINA1- | TCAGCCTTTCTC | 2/0 asymmetric bulge at +8 position (GA-) | ADAR1 specificity: |
| 315 | 12_34_ | GTCGAACGTCA | 0/2 asymmetric bulge at +23 position (-AC) | 1.43 |
| | 45.34 | | | |
| SEQ | SERP_ | ACATGGCCCCA | 0/3 asymmetric bulge at -10 position (-CUA) | ADAR1 on target: |
| ID | 031836_ | GAGCTTCAGTG | 1/1 A/C mismatch at 0 position | 42.20% |
| NO: | SERINA1- | CCCTTTCTCGTC | 0/1 asymmetric bulge at +8 position (-G) | ADAR1 specificity: |
| 316 | 15_31_ | GATGGTCCTAA | 1/0 asymmetric bulge at +18 position (G-) | 1.39 |
| | 45.31 | GCA | | |
| SEQ | SERP_ | CTCTAAAAACA | 1/1 C/A mismatch at -1 position | ADAR1 on target: |
| ID | 033465_ | TGACCCCAGCA | 4/3 asymmetric bulge at +2 position (GAAA- | 43.60% |
| NO: | SERINA1- | GCTTCAGTCCT | CCG) | ADAR1 specificity: |
| 317 | 7_39_ | CCGTTATCGAT | 1/1 G/U wobble base pair at +6 position | 1.42 |
| | 45.39 | | 1/1 C/A mismatch at +25 position | |
| SEQ | SERP_ | ACATCAGGCCC | 1/1 A/A mismatch at -9 position | ADAR1 on target: |
| ID | 034669_ | CACAGCTTCAG | 1/1 A/C mismatch at 0 position | 44.70% |
| NO | SERINA1- | TCCCTTCTCGTC | 1/0 asymmetric bulge at +5 position (A-) | ADAR1 specificity: |
| 318 | 15_31_ | GATGGACAG | 1/0 asymmetric bulge at +19 position (C-) | 1.19 |
| | 45.31 | | 0/2 asymmetric bulge at +26 position (-CA) | |
| SEQ | SERP_ | CCCCAGCGTCT | 1/0 asymmetric bulge at -14 position (U-) | ADAR1 on target: |
| ID | 034803_ | TCAGTCCCTTTC | 1/1 C/A mismatch at -12 position | 49.00% |
| NO: | SERINA1- | TCGTCGATGGT | 1/1 U/G wobble base pair at -11 position | ADAR1 specificity: |
| 319 | 21_25_ | CGACCAGCCT | 1/1 A/C mismatch at 0 position | 1.18 |
| | 45.25 | | 1/1 C/U mismatch at +16 position | |
| | | | 1/1 U/G wobble base pair at +17 position | |
| SEQ | SERP_ | GGCCCCAGCAG | 1/1 A/C mismatch at 0 position | ADAR1 on target: |
| ID | 034837_ | CTTCAGTCCCA | 3/3 symmetric bulge at +3 position (AAA- | 48.00% |
| NO: | SERINA1- | GACTCGTCGAT | AGA) | ADAR1 specificity: |
| 320 | 19_27_ | GGTCAGCACAA | | 1.37 |
| | 45.27 | C | | |
| SEQ | SERP_ | TAAAAAACATG | 1/1 A/C mismatch at 0 position | ADAR1 on target: |
| ID | 035444_ | GCCCCAGCAGT | 0/1 asymmetric bulge at +2 position (-A) | 42.60% |
| NO | SERINA1- | CAGTCCCTTTA | 2/0 asymmetric bulge at +13 position (AA-) | ADAR1 specificity: |
| 321 | 10_36_ | CTCGTCGATGG | 1/1 G/U wobble base pair at +15 position | 1.32 |
| | 45.36 | T | | |
| SEQ | SERP_ | GGCCAGCAGCT | 3/3 symmetric bulge at -6 position (CCA- | ADAR1 on target: |
| ID | 036632_ | TCACCAAGTTT | AUA) | 60.10% |
| NO: | SERINA1- | CTCGTCGAATA | 1/1 A/C mismatch at 0 position | ADAR1 specificity: |
| 322 | 21_25_ | TCAGCACAGCC | 5/5 symmetric internal loop at +6 position | 1.54 |
| | 45.25 | T | (GGGAC-CCAAG) | |
| SEQ | SERP_ | CTAAAAACATG | 3/3 symmetric bulge at -1 position spanning | ADAR1 on target: |
| ID | 039699_ | GCCCCAGCAGC | 0 position (CAA-GAU) | 63.60% |
| NO: | SERINA1- | TTCAGTCAACT | 0/1 asymmetric bulge at +1 position (-C) | ADAR1 specificity: |
| 323 | 9_37_ | TCTCGATTCGA | 1/2 asymmetric bulge at +7 position (G-AA) | 1.63 |
| | 745.3 | TGG | | |
| SEQ | SERP_ | TGGCGCCCCAG | 0/2 asymmetric bulge at -7 position (-CU) | ADAR1 on target: |
| ID | 040418_ | CAGCTTCATCC | 0/2 asymmetric bulge at -3 position (-GG) | 48.20% |
| NO: | SERINA1- | CTTCTCGTCGG | 1/1 A/C mismatch at 0 position | ADAR1 specificity: |
| 324 | 18_28_ | GATGCTGTCAG | 1/0 asymmetric bulge at +5 position (A-) | 1.45 |
| | 45.28 | CACAG | 1/0 asymmetric bulge at +10 position (C-) | |
| | | | 0/1 asymmetric bulge at +25 position (-C) | |

### Example 26

### Engineered Guide RNAs Targeting SERPINA1 mRNA

This example describes engineered guide RNAs that target SERPINA1 mRNA. A SERPINA1 guide RNA was identified from a high throughput screen for engineered guide RNAs against SERPINA1, similar to the screen described in EXAMPLE 25. Self-annealing RNA structures comprising engineered guide RNA sequences and the sequences of the regions targeted by the guide RNAs were contacted with an RNA editing entity (e.g., a recombinant ADAR1 and/or ADAR2) for 30 minutes under conditions that allow for the editing of the regions targeted by the guide RNAs. The regions targeted by the guide RNAs were subsequently assessed for editing using next generation sequencing (NGS).

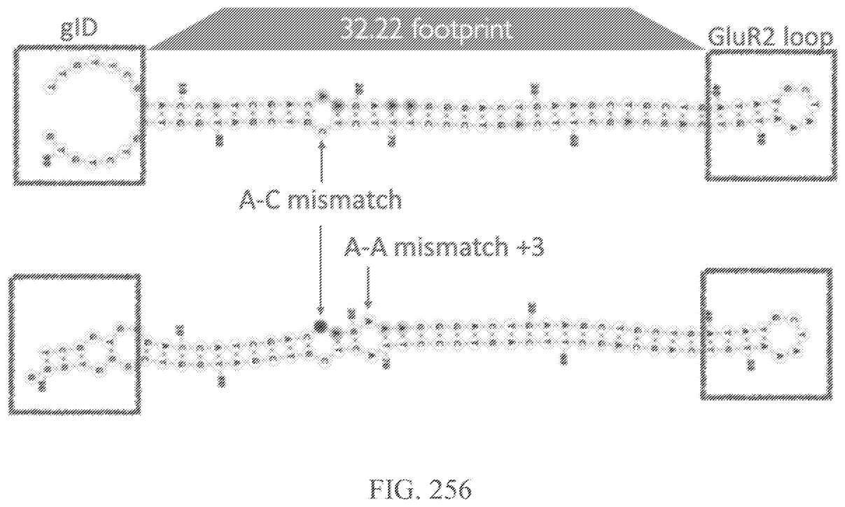

FIG. 256 shows a depiction of a first engineered guide RNA (at top) that forms a single mismatch with the target SERPINA1 mRNA sequence and a second exemplary engineered guide RNA (at bottom) targeting SERPINA1 mRNA, where the second engineered guide RNA forms two mismatches with the target SERPINA1 mRNA sequence. A summary of each engineered guide RNA is described below in TABLE 30. The column in TABLE 30 titled “Structural Features” describes structural features in the double stranded RNA substrate formed upon hybridization of the gRNA to the target RNA.

TABLE 30
Exemplary engineered guide RNA sequences that target SERPINA1

| SEQ | | | | |
|---|---|---|---|---|
| ID | | | Structural Features | |
| NO | Name | Sequence | (target/guide) | Metrics |
| SEQ | SerpRandom_ | GACCATCGACAAGAAAGG | 1 A/C mismatch | 14% on target |
| ID | ID38633_ | GACTGAAGCTGCTGATAT | | Specificity = 0.69 |
| NO: | count852 | GCTAAATGTCAGCAGCT | | |
| 346 | | TCAGTCCCTTTCTCGTCGA | | |
| | | TGGTC | | |
| SEQ | SerpRandom_ | GACCATCGACAAGAAAGG | 1 A/C mismatch | 35% on target |
| ID | ID26156_ | GACTGAAGCTGCTGATAT | 1 A/A mismatch | Specificity = 0.92 |
| NO: | count1057 | GCTAAATGTCAGCAGCTT | | |
| 347 | | CAGTCCCTTACTCGTCGAT | | |
| | | GGTC | | |

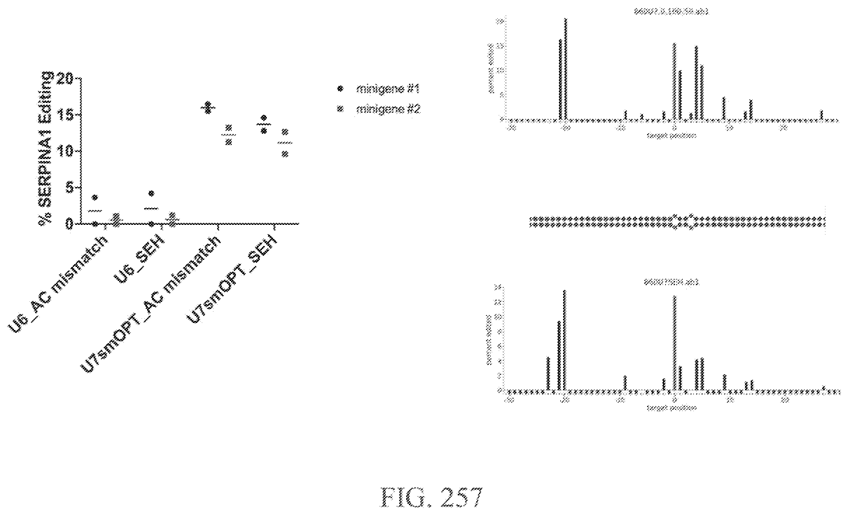

The engineered guide RNA sequences in TABLE 30 were adapted for in vitro testing in cells. The engineered guide RNA sequences in TABLE 30 were adapted to 100mer sequences, as disclosed in TABLE 31. K562 cells were stably transfected with a Piggybac vector containing one of two SERPINA1 minigene constructs containing the E342K mutation. Engineered guide RNA sequences were plasmid transfected (2 μg) into 2×10\hat{ }5 K562 cells. Plasmids encoded for the engineered guide RNA sequence plus a downstream mouse U7 hairpin (CAGGTTTTCTGACTTCGGTCGGAAAACCCCT; SEQ ID NO: 389) and SmOPT sequence (AATTTTTGGAG; SEQ ID NO: 390) and expression was driven under a U6 promoter (no U7 hairpin or SmOPT sequence) or a U7 promoter. 24 hours post-transfection, RNA was isolated, converted to cDNA and Sanger sequenced. FIG. 257 (at left) demonstrated that the constructs containing the U7 promoter, U7 hairpin, and the SmOPT sequences exhibited the highest levels of on-target RNA editing. FIG. 257 (at right) demonstrated that the second engineered guide RNA, which formed an A/C and A/A mismatch upon hybridization to SERPINA1 mRNA, exhibited less local off-target editing.

TABLE 31
Exemplary engineered guide RNA sequences that target SERPINA1

| SEQ | | | | |
|---|---|---|---|---|
| ID | | | Structural Features | Metrics (Replicate |
| NO | Name | Sequence | (target/guide) | values) |
| SEQ | 0,100,50_AC | ATGGGTATGGCCTCTAAA | 1 A/C mismatch | 15.53% and 16.43% |
| ID | mismatch | AACATGGCCCCAGCAGCT | | |
| NO: | | TCAGTCCCTTTCTCGTCGA | | |
| 348 | | TGGTCAGCACAGCCTTAT | | |
| | | GCACGGCCTGGAGGGGAG | | |
| | | AGAAGCAGA | | |
| SEQ | 0,100,50_SEH | ATGGGTATGGCCTCTAAA | 1 A/C mismatch | 12.83% and 14.62% |
| ID | | AACATGGCCCCAGCAGCT | 1 A/A mismatch | |
| NO: | | TCAGTCCCTTACTCGTCGA | | |
| 349 | | TGGTCAGCACAGCCTTAT | | |
| | | GCACGGCCTGGAGGGGAG | | |
| | | AGAAGCAGA | | |

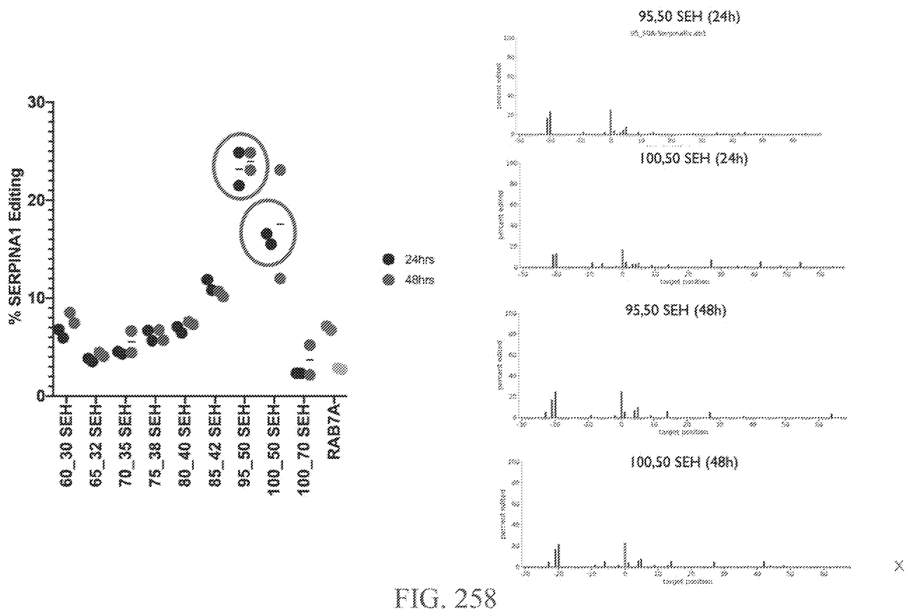

The engineered guide RNA sequence of SEQ ID NO: 349 in TABLE 31 was carried forward into further experiments where the structural features of the A/C mismatch and A/A mismatch were preserved, but the overall engineered guide RNA length was shortened to 95, 85, 80, 75, 70, 65, and 60 nucleotides long. Sequences of the engineered guide RNA sequences used in this experiment are described in TABLE 32. Cell experiments were run as described above, but with RNA editing assessed at 24 hours and 48 hours post-transfection. As demonstrated in FIG. 258 (at left), the engineered guide sequence with a length of 95 nucleotides exhibited the highest percentage of SERPINA1 mRNA editing. All engineered guide RNA sequences were tested in constructs containing a U7 hairpin and a SmOPT sequence, with expression driven via a U1 promoter.

TABLE 32
Exemplary engineered guide RNA sequences that target SERPINA1

| SEQ | | | | |
|---|---|---|---|---|
| ID | | | Structural Features | Metrics (Replicate |
| NO | Name | Sequence | (target/guide) | values) |
| SEQ | 60_30 SEH | CATGGCCCCAGCAGCTTC | 1 A/C mismatch | 6.81% & 5.95% |
| ID | | AGTCCCTTACTCGTCGATG | 1 A/A mismatch | |
| NO: | | GTCAGCACAGCCTTATGC | | |
| 350 | | ACGGC | | |
| SEQ | 65_32 SEH | AACATGGCCCCAGCAGCT | 1 A/C mismatch | 3.84% & 3.53% |
| ID | | TCAGTCCCTTACTCGTCGA | 1 A/A mismatch | |
| NO: | | TGGTCAGCACAGCCTTAT | | |
| 351 | | GCACGGCCTG | | |
| SEQ | 70_35 SEH | AAAAACATGGCCCCAGCA | 1 A/C mismatch | 4.29% & 4.56% |
| ID | | GCTTCAGTCCCTTACTCGT | 1 A/A mismatch | |
| NO: | | CGATGGTCAGCACAGCCT | | |
| 352 | | TATGCACGGCCTGGA | | |
| SEQ | 75_48 SEH | TCTAAAAACATGGCCCCA | 1 A/C mismatch | 6.71% & 5.66% |
| ID | | GCAGCTTCAGTCCCTTACT | 1 A/A mismatch | |
| NO: | | CGTCGATGGTCAGCACAG | | |
| 353 | | CCTTATGCACGGCCTGGA | | |
| | | GG | | |
| SEQ | 80_40 SEH | CCTCTAAAAACATGGCCC | 1 A/C mismatch | 7.08% & 6.45% |
| ID | | CAGCAGCTTCAGTCCCTTA | 1 A/A mismatch | |
| NO: | | CTCGTCGATGGTCAGCAC | | |
| 354 | | AGCCTTATGCACGGCCTG | | |
| | | GAGGGGA | | |
| SEQ | 85_42 SEH | GGCCTCTAAAAACATGGC | 1 A/C mismatch | 11.89% & 10.82% |
| ID | | CCCAGCAGCTTCAGTCCCT | 1 A/A mismatch | |
| NO: | | TACTCGTCGATGGTCAGC | | |
| 355 | | ACAGCCTTATGCACGGCC | | |
| | | TGGAGGGGAGAG | | |
| SEQ | 95_50 SEH | ATGGGTATGGCCTCTAAA | 1 A/C mismatch | 24.87% & 21.5% |
| ID | | AACATGGCCCCAGCAGCT | 1 A/A mismatch | |
| NO: | | TCAGTCCCTTACTCGTCGA | | |
| 356 | | TGGTCAGCACAGCCTTAT | | |
| | | GCACGGCCTGGAGGGGAG | | |
| | | AGAA | | |
| SEQ | 100_50 SEH | ATGGGTATGGCCTCTAAA | 1 A/C mismatch | 15.49% & 16.58% |
| ID | | AACATGGCCCCAGCAGCT | 1 A/A mismatch | |
| NO: | | TCAGTCCCTTACTCGTCGA | | |
| 349 | | TGGTCAGCACAGCCTTAT | | |
| | | GCACGGCCTGGAGGGGAG | | |
| | | AGAAGCAGA | | |
| SEQ | 100_70 SEH | TGACCTCGGGGGGGATAG | 1 A/C mismatch | 2.5% & 2.87% |
| ID | | ACATGGGTATGGCCTCTA | 1 A/A mismatch | |
| NO: | | AAAACATGGCCCCAGCAG | | |
| 358 | | CTTCAGTCCCTTACTCGTC | | |
| | | GATGGTCAGCACAGCCTT | | |
| | | ATGCACGGC | | |

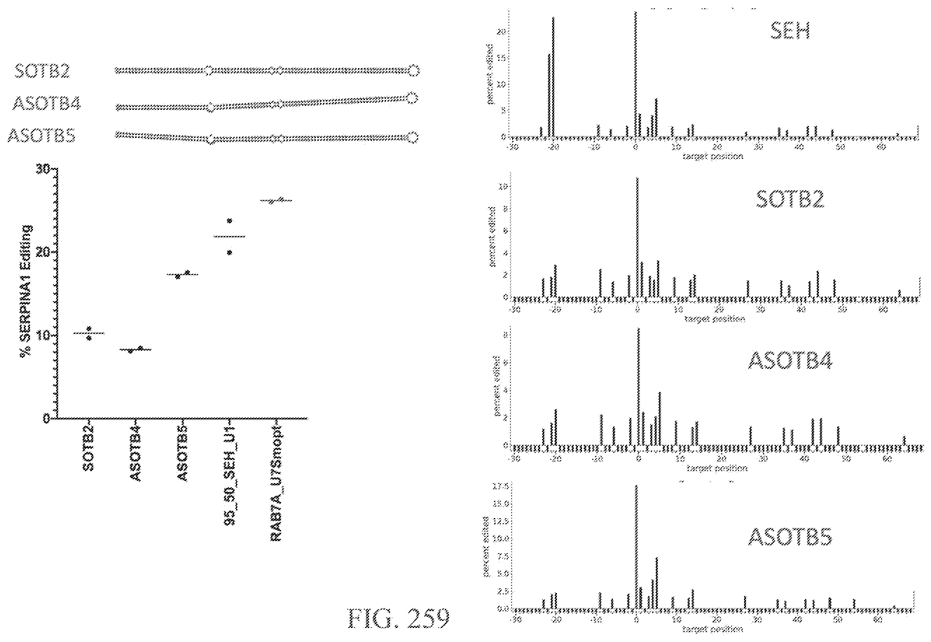

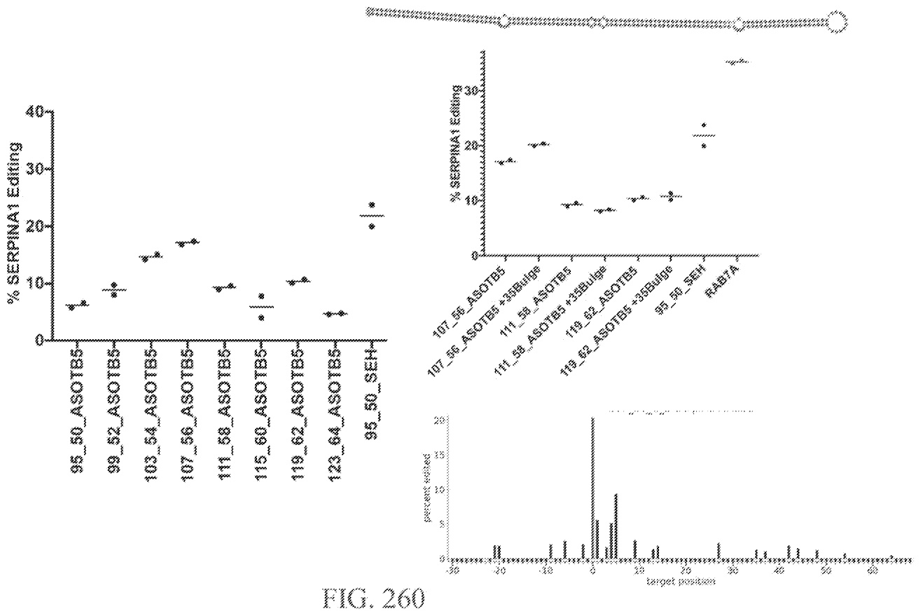

The engineered guide RNAs that were 95 nucleotides in length were carried forward into further experiments in cells. Cell experiments were run as described above, but with RNA editing assessed at 24 hours post-transfection. To examine the ability to reduce local off-target editing, guide RNAs were further engineered to form a symmetric, an asymmetric bulge, or an asymmetric internal loop at sites of local off-target editing (−20 and −21 positions relative to the target adenosine). Sequences of the engineered guide RNA sequences used in this experiment are described in TABLE 33. As shown in FIG. 259 (at left), SEQ ID NO: 361 (ASOTB) and SEQ ID NO: 362 (95_50_SEH_U1) exhibited the highest levels of on-target SERPINA1 RNA editing. As shown in FIG. 259 (at right), the presence of a symmetric bulge, asymmetric bulge, or asymmetric loop in the double stranded RNA substrate formed by the target RNA and the engineered guide RNA reduced local off-target editing. Thus, the engineered guide RNA of SEQ ID NO: 361 (ASOTB5) displayed the highest level of on-target editing while minimizing local off-target editing. Rab7A editing by a RAM7A guide RNA was tested as a positive control. All engineered guide RNA sequences were tested in constructs containing a U7 hairpin and a SmOPT sequence, with expression driven via a U1 promoter.

TABLE 33
Exemplary engineered guide RNA sequences that target SERPINA1

| SEQ | | | | Metrics |
|---|---|---|---|---|
| ID | | | Structural Features | (Replicate |
| NO | Name | Sequence | (target/guide) | values) |
| SEQ | SOTB2 | TATGGCCTCTAAAAACAT | 1 A/C mismatch | 10.8%, 9.7% |
| ID | | GGCCCCAGCAGCTTCAGT | 1 A/A mismatch | |
| NO: | | CCCTTACTCGTCGATGGTC | 2/2 symmetric bulge | |
| 359 | | AGCACAGCCAAATGCACG | | |
| | | GCCTGGAGGGGAGAGAAG | | |
| | | CAGA | | |
| SEQ | ASOTB4 | TATGGCCTCTAAAAACAT | 1 A/C mismatch | 8.48%, 8.1% |
| ID | | GGCCCCAGCAGCTTCAGT | 1 A/A mismatch | |
| NO: | | CCCTTACTCGTCGATGGTC | 1/4 asymmetric bulge | |
| 360 | | AGCACAGCCCCAGTATGC | | |
| | | ACGGCCTGGAGGGGAGAG | | |
| | | AAGCAGA | | |
| SEQ | ASOTB5 | TATGGCCTCTAAAAACAT | 1 A/C mismatch | 17.04%, 17.57% |
| ID | | GGCCCCAGCAGCTTCAGT | 1 A/A mismatch | |
| NO: | | CCCTTACTCGTCGATGGTC | 1/5 asymmetric | |
| 361 | | AGCACAGCCAGTCGTATG | internal loop | |
| | | CACGGCCTGGAGGGGAGA | | |
| | | GAAGCAGA | | |
| SEQ | 95_50_SEH_ | ATGGGTATGGCCTCTAAA | 1 A/C mismatch | 19.95%, 23.75% |
| ID | U1 | AACATGGCCCCAGCAGCT | 1 A/A mismatch | |
| NO: | | TCAGTCCCTTACTCGTCGA | | |
| 362 | | TGGTCAGCACAGCCTTAT | | |
| | | GCACGGCCTGGAGGGGAG | | |
| | | AGAA | | |

The engineered guide RNAs that formed the asymmetric internal loop described above and in TABLE 33 were carried forward into further experiments in cells where guide length was once again modulated. Cell experiments were run as described above, but with RNA editing assessed at 24 hours post-transfection. Sequences of the engineered guide RNA sequences used in this experiment are described in TABLE 34. As shown in FIG. 260 (at left), on-target editing was increased by increasing length (e.g., engineered guide RNAs of length 107, 111, and 119 nucleotides). All engineered guide RNA sequences were tested in constructs containing a U7 hairpin and a SmOPT sequence, with expression driven via a U1 promoter.

TABLE 34
Exemplary engineered guide RNA sequences that target SERPINA1

| SEQ | | | | Metrics |
|---|---|---|---|---|
| ID | | | Structural Features | (replicate |
| NO | Name | Sequence | (target/guide) | values) |
| SEQ | 95_50_ASOTB5 | ATGGGTATGGCCTCTAAA | 1 A/C mismatch | 5.76% & 6.64% |
| ID | | AACATGGCCCCAGCAGCT | 1 A/A mismatch | |
| NO: | | TCAGTCCCTTACTCGTCGA | 1/5 asymmetric | |
| 364 | | TGGTCAGCACAGCCAGTC | internal loop | |
| | | GTATGCACGGCCTGGAGG | | |
| | | GGAGAGAA | | |
| SEQ | 99_52_ASOTB5 | ACATGGGTATGGCCTCTA | 1 A/C mismatch | 9.727% & 8.007% |
| ID | | AAAACATGGCCCCAGCAG | 1 A/A mismatch | |
| NO: | | CTTCAGTCCCTTACTCGTC | 1/5 asymmetric | |
| 365 | | GATGGTCAGCACAGCCAG | internal loop | |
| | | TCGTATGCACGGCCTGGA | | |
| | | GGGGAGAGAAGC | | |
| SEQ | 103_54_ASOTB5 | AGACATGGGTATGGCCTC | 1 A/C mismatch | 14.187% & 15.094% |
| ID | | TAAAAACATGGCCCCAGC | 1 A/A mismatch | |
| NO: | | AGCTTCAGTCCCTTACTCG | 1/5 asymmetric | |
| 366 | | TCGATGGTCAGCACAGCC | internal loop | |
| | | AGTCGTATGCACGGCCTG | | |
| | | GAGGGGAGAGAAGCAG | | |
| SEQ | 107_56_ASOTB5 | ATAGACATGGGTATGGCC | 1 A/C mismatch | 16.84% & 14.39% |
| ID | | TCTAAAAACATGGCCCCA | 1 A/A mismatch | |
| NO: | | GCAGCTTCAGTCCCTTACT | 1/5 asymmetric | |
| 367 | | CGTCGATGGTCAGCACAG | internal loop | |
| | | CCAGTCGTATGCACGGCC | | |
| | | TGGAGGGGAGAGAAGCAG | | |
| | | AG | | |
| SEQ | 111_58_ | GGATAGACATGGGTATGG | 1 A/C mismatch | 9.59% & 8.977% |
| ID | ASOTB5 | CCTCTAAAAACATGGCCC | 1 A/A mismatch | |
| NO: | | CAGCAGCTTCAGTCCCTTA | 1/5 asymmetric | |
| 368 | | CTCGTCGATGGTCAGCAC | internal loop | |
| | | AGCCAGTCGTATGCACGG | | |
| | | CCTGGAGGGGAGAGAAGC | | |
| | | AGAGAC | | |
| SEQ | 115_60_ | GGGGATAGACATGGGTAT | 1 A/C mismatch | 7.786% & 3.99% |
| ID | ASOTB5 | GGCCTCTAAAAACATGGC | 1 A/A mismatch | |
| NO: | | CCCAGCAGCTTCAGTCCCT | 1/5 asymmetric | |
| 369 | | TACTCGTCGATGGTCAGC | internal loop | |
| | | ACAGCCAGTCGTATGCAC | | |
| | | GGCCTGGAGGGGAGAGAA | | |
| | | GCAGAGACAC | | |
| SEQ | 119_62_ | GGGGGGATAGACATGGGT | 1 A/C mismatch | 13.12% & 10.69% |
| ID | ASOTB5 | ATGGCCTCTAAAAACATG | 1 A/A mismatch | |
| NO: | | GCCCCAGCAGCTTCAGTC | 1/5 asymmetric | |
| 370 | | CCTTACTCGTCGATGGTCA | internal loop | |
| | | GCACAGCCAGTCGTATGC | | |
| | | ACGGCCTGGAGGGGAGAG | | |
| | | AAGCAGAGACACGT | | |
| SEQ | 123_64_5 | CGGGGGGGATAGACATGG | 1 A/C mismatch | 4.75% & 4.61% |
| ID | ASOTB | GTATGGCCTCTAAAAACA | 1 A/A mismatch | |
| NO: | | TGGCCCCAGCAGCTTCAG | 1/5 asymmetric | |
| 371 | | TCCCTTACTCGTCGATGGT | internal loop | |
| | | CAGCACAGCCAGTCGTAT | | |
| | | GCACGGCCTGGAGGGGAG | | |
| | | AGAAGCAGAGACACGTTG | | |
| SEQ | 95_50_SEH | ATGGGTATGGCCTCTAAA | 1 A/C mismatch | 21.5% & 24.87% |
| ID | | AACATGGCCCCAGCAGCT | 1 A/A mismatch | |
| NO: | | TCAGTCCCTTACTCGTCGA | | |
| 362 | | TGGTCAGCACAGCCTTAT | | |
| | | GCACGGCCTGGAGGGGAG | | |
| | | AGAA | | |

The engineered guide RNAs that were 107, 111, and 119 nucleotides long (see above and in TABLE 34) were carried forward into further experiments in cell where an additional bulge was placed at the +35 position relative to the target adenosine to be edited. Cell experiments were run as described above, but with RNA editing assessed at 24 hours post-transfection. Sequences of the engineered guide RNA sequences used in this experiment are described in TABLE 35. As shown in FIG. 260 (at right), a preferred gRNA length exists that drives an ideal delta G/binding affinity of the guide-target RNA scaffold, which enhances editing. A delta G that is too high is not ideal for efficient editing. Sanger sequencing data is shown in FIG. 260 for SEQ ID NO: 374. Rab7A editing by a RAB7A guide RNA was tested as a positive control. All engineered guide RNA sequences were tested in constructs containing a U7 hairpin and a SmOPT sequence, with expression driven via a U1 promoter.

TABLE 35
Exemplary engineered guide RNA sequences that target SERPINA1

| SEQ | | | | Metrics |
|---|---|---|---|---|
| ID | | | Structural Features | (Replicate |
| NO | Name | Sequence | (target/guide) | values) |
| SEQ | 107_56_ | ATAGACATGGGTATGGCC | 1 A/C mismatch | 16.84% & 14.39% |
| ID | 5ASOTB | TCTAAAAACATGGCCCCA | 1 A/A mismatch | |
| NO: | | GCAGCTTCAGTCCCTTACT | 1/5 asymmetric | |
| 373 | | CGTCGATGGTCAGCACAG | internal loop | |
| | | CCAGTCGTATGCACGGCC | | |
| | | TGGAGGGGAGAGAAGCAG | | |
| | | AG | | |
| SEQ | 107_56_ | ATAGACATGGGTATGGCC | 1 A/C mismatch | 19.982% & 20.39% |
| ID | ASOTB5 + | TCTCCACAAAACATGGCC | 1 A/A mismatch | |
| NO: | 35Bulge | CCAGCAGCTTCAGTCCCTT | 1/5 asymmetric | |
| 374 | | ACTCATCGATGGTCAGCA | internal loop | |
| | | CAGCCAGTCGTATGCACG | 1/4 asymmetric bulge | |
| | | GCCTGGAGGGGAGAGAAG | | |
| | | CAGAG | | |
| SEQ | 111_58_ | GGATAGACATGGGTATGG | 1 A/C mismatch | 9.59% & 8.977% |
| ID | ASOTB5 | CCTCTAAAAACATGGCCC | 1 A/A mismatch | |
| NO: | | CAGCAGCTTCAGTCCCTTA | 1/5 asymmetric | |
| 375 | | CTCGTCGATGGTCAGCAC | internal loop | |
| | | AGCCAGTCGTATGCACGG | | |
| | | CCTGGAGGGGAGAGAAGC | | |
| | | AGAGAC | | |
| SEQ | 111_58_ | GGATAGACATGGGTATGG | 1 A/C mismatch | 8.398% & 8.08% |
| ID | ASOTB5 + | CCTCTCCACAAAACATGG | 1 A/A mismatch | |
| NO: | 35Bulge | CCCCAGCAGCTTCAGTCCC | 1/5 asymmetric | |
| 376 | | TTACTCATCGATGGTCAGC | internal loop | |
| | | ACAGCCAGTCGTATGCAC | 1/4 asymmetric bulge | |
| | | GGCCTGGAGGGGAGAGAA | | |
| | | GCAGAGAC | | |
| SEQ | 119_62_ | GGGGGGATAGACATGGGT | 1 A/C mismatch | 13.12% & 10.69% |
| ID | ASOTB5 | ATGGCCTCTAAAAACATG | 1 A/A mismatch | |
| NO: | | GCCCCAGCAGCTTCAGTC | 1/5 asymmetric | |
| 377 | | CCTTACTCGTCGATGGTCA | internal loop | |
| | | GCACAGCCAGTCGTATGC | | |
| | | ACGGCCTGGAGGGGAGAG | | |
| | | AAGCAGAGACACGT | | |
| SEQ | 119_62_ | GGGGGGATAGACATGGGT | 1 A/C mismatch | 11.36% & 10.18% |
| ID | ASOTB5_ | ATGGCCTCTCCACAAAAC | 1 A/A mismatch | |
| NO: | 35Bulge | ATGGCCCCAGCAGCTTCA | 1/5 asymmetric | |
| 378 | | GTCCCTTACTCATCGATGG | internal loop | |
| | | TCAGCACAGCCAGTCGTA | 1/4 asymmetric bulge | |
| | | TGCACGGCCTGGAGGGGA | | |
| | | GAGAAGCAGAGACACGT | | |
| SEQ | 95_50_SEH | ATGGGTATGGCCTCTAAA | 1 A/C mismatch | 21.5% & 24.87% |
| ID | | AACATGGCCCCAGCAGCT | 1 A/A mismatch | |
| NO: | | TCAGTCCCTTACTCGTCGA | | |
| 362 | | TGGTCAGCACAGCCTTAT | | |
| | | GCACGGCCTGGAGGGGAG | | |
| | | AGAA | | |
| SEQ | RAB7A | TGATAAAAGGCGTACATA | | 35.467% & 35.026% |
| ID | | AGTCTTGTGTCTACTGTAC | | |
| NO: | | AGAAGACTGCCGCCAGCT | | |
| 380 | | GGATTTCCCAATTCTGAGT | | |
| | | AACACTCTGCAATCCAAA | | |
| | | CAGGGTTC | | |

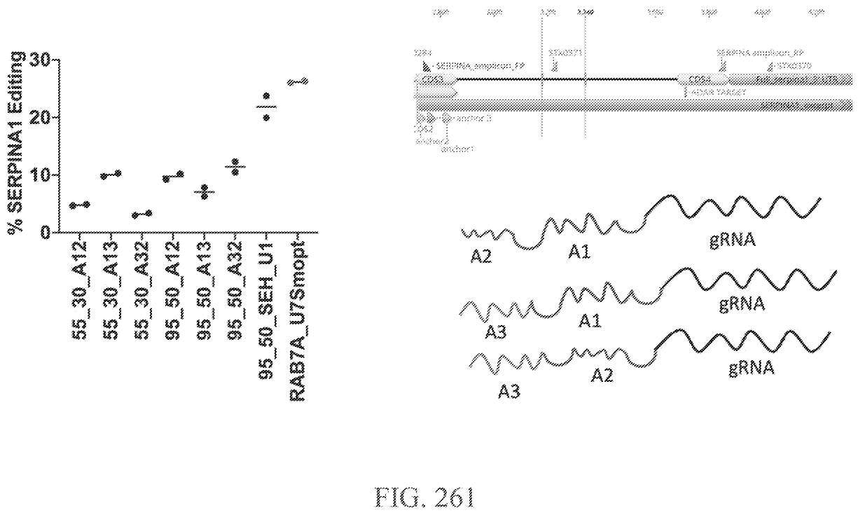

Polynucleotides encoding engineered guide RNAs and oligo tethers were tested for SERPINA1 RNA editing of the E342K. Cell experiments were run as described above, but with RNA editing assessed at 24 hours post-transfection. Sequences of the engineered guide RNA sequences used in this experiment are described in TABLE 36. FIG. 261 (at right) shows a schematic of the SERPINA1 target sequence and oligo tether engineered guide RNAs. As shown in FIG. 261 (at left), polynucleotides encoding engineered guide RNAs and oligo tethers elicited RNA editing of SERPINA1 mRNA. Rab7A editing by a RAB7A guide RNA was tested as a positive control. All engineered guide RNA sequences were tested in constructs containing a U7 hairpin and a SmOPT sequence, with expression driven via a U1 promoter.

TABLE 36
Exemplary engineered guide RNA sequences that target SERPINA1

| SEQ | | | | Metrics |
|---|---|---|---|---|
| ID | | | Structural Features | (replicate |
| NO | Name | Sequence | (target/guide) | values) |
| SEQ | 55_30_A12 | CCCCAGCAGCTTCAGTCCC | 1 A/C mismatch | 4.72% & 4.92% |
| ID | | TTACTCGTCGATGGTCAGC | 1 A/A mismatch | |
| NO: | | ACAGCCTTATGCACGGCC | 65/8 asymmetric loop | |
| 381 | | CAGTGTCTCCTCTGTGACC | | |
| | | CCGGAGAGGTCAGCCCCA | | |
| | | CCAGTGTCGGACAGTTTG | | |
| | | GGTAAATGTAAGCTGGCA | | |
| | | GA | | |
| SEQ | 55_30_A13 | CCCCAGCAGCTTCAGTCCC | 1 A/C mismatch | 9.78% & 10.35% |
| ID | | TTACTCGTCGATGGTCAGC | 1 A/A mismatch | |
| NO: | | ACAGCCTTATGCACGGCC | 28/8 asymmetric loop | |
| 382 | | CAGTGTCTCCTCTGTGACC | | |
| | | CCGGAGAGGTCAGCCCCA | | |
| | | CCAGTGTCGACCCAGGAC | | |
| | | GCTCTTCAGATCATAGGTT | | |
| | | C | | |
| SEQ | 55_30_A32 | CCCCAGCAGCTTCAGTCCC | 1 A/C mismatch | 3.4% & 3.01% |
| ID | | TTACTCGTCGATGGTCAGC | 1 A/A mismatch | |
| NO: | | ACAGCCTTATGCACGGCC | 7/8 asymmetric loop | |
| 383 | | CAGTGTCGACCCAGGACG | | |
| | | CTCTTCAGATCATAGGTTC | | |
| | | CCAGTGTCGGACAGTTTG | | |
| | | GGTAAATGTAAGCTGGCA | | |
| | | GA | | |
| SEQ | 95_50_A12 | TATGGCCTCTAAAAACAT | 1 A/C mismatch | 9.27% & 10.23% |
| ID | | GGCCCCAGCAGCTTCAGT | 1 A/A mismatch | |
| NO: | | CCCTTACTCGTCGATGGTC | 65/8 asymmetric loop | |
| 384 | | AGCACAGCCTTATGCACG | | |
| | | GCCTGGAGGGGAGAGAAG | | |
| | | CAGACCAGTGTCTCCTCTG | | |
| | | TGACCCCGGAGAGGTCAG | | |
| | | CCCCACCAGTGTCGGACA | | |
| | | GTTTGGGTAAATGTAAGC | | |
| | | TGGCAGA | | |
| SEQ | 95_50_A13 | TATGGCCTCTAAAAACAT | 1 A/C mismatch | 7.83% & 6.32% |
| ID | | GGCCCCAGCAGCTTCAGT | 1 A/A mismatch | |
| NO: | | CCCTTACTCGTCGATGGTC | 28/8 asymmetric loop | |
| 385 | | AGCACAGCCTTATGCACG | | |
| | | GCCTGGAGGGGAGAGAAG | | |
| | | CAGACCAGTGTCTCCTCTG | | |
| | | TGACCCCGGAGAGGTCAG | | |
| | | CCCCACCAGTGTCGACCC | | |
| | | AGGACGCTCTTCAGATCA | | |
| | | TAGGTTC | | |
| SEQ | 95_50_A32 | TATGGCCTCTAAAAACAT | 1 A/C mismatch | 10.54% & 12.34% |
| ID | | GGCCCCAGCAGCTTCAGT | 1 A/A mismatch | |
| NO: | | CCCTTACTCGTCGATGGTC | 7/8 asymmetric loop | |
| 386 | | AGCACAGCCTTATGCACG | | |
| | | GCCTGGAGGGGAGAGAAG | | |
| | | CAGACCAGTGTCGACCCA | | |
| | | GGACGCTCTTCAGATCAT | | |
| | | AGGTTCCCAGTGTCGGAC | | |
| | | AGTTTGGGTAAATGTAAG | | |
| | | CTGGCAGA | | |
| SEQ | 95_50_SEH_ | ATGGGTATGGCCTCTAAA | 1 A/C mismatch | 19.95% & 23.75% |
| ID | U1 | AACATGGCCCCAGCAGCT | 1 A/A mismatch | |
| NO: | | TCAGTCCCTTACTCGTCGA | | |
| 387 | | TGGTCAGCACAGCCTTAT | | |
| | | GCACGGCCTGGAGGGGAG | | |
| | | AGAA | | |
| SEQ | RAB7A_ | TGATAAAAGGCGTACATA | 1 A/C mismatch | 26.35 and 26.04 |
| ID | U7Smopt | AGTCTTGTGTCTACTGTAC | | |
| NO: | | AGAAGACTGCCGCCAGCT | | |
| 388 | | GGATTTCCCAATTCTGAGT | | |
| | | AACACTCTGCAATCCAAA | | |
| | | CAGGGTTC | | |

While preferred embodiments of the present disclosure have been shown and described herein, such embodiments are provided by way of example only. Numerous variations, changes, and substitutions can occur without departing from the present disclosure. It should be understood that various alternatives to the embodiments described herein may be employed.

1. An engineered guide RNA that, upon hybridization to a target RNA implicated in a disease or condition, forms a guide-target RNA scaffold comprising a structural feature selected from the group consisting of a bulge, an internal loop, a hairpin, and any combination thereof, wherein the structural feature substantially forms upon hybridization to the target RNA.

2. The engineered guide RNA of claim 1, wherein the guide-target RNA scaffold further comprises a mismatch.

3. The engineered guide RNA of claim 2, wherein the mismatch is an adenosine/cytosine (A/C) mismatch, wherein the adenosine (A) is present in the target RNA and the cytosine (C) is present in the engineered guide RNA.

4. The engineered guide RNA of any one of claims 1-3, wherein the guide-target RNA scaffold comprises a wobble base pair.

5. The engineered guide RNA of any one of claims 1-3, wherein the guide-target RNA scaffold is a substrate for an RNA editing entity that chemically modifies a base of a nucleotide in the target RNA.

6. The engineered guide RNA of any one of claims 3-5, wherein the RNA editing entity chemically modifies the adenosine in the target RNA to an inosine.

7. The engineered guide RNA of any one of claims 1-6, wherein the guide-target RNA scaffold comprises a structured motif comprising two or more structural features selected from the group consisting of a bulge, an internal loop, a hairpin, and any combination thereof.

8. The engineered guide RNA of any one of claims 1-6, wherein the guide-target RNA scaffold comprises at least two, three, four, five, six, seven, eight, nine, or 10 structural features selected from the group consisting of a bulge, an internal loop, a hairpin, and any combination thereof.

9. The engineered guide RNA of any one of claims 1-8, wherein the structural feature is a bulge.

10. The engineered guide RNA of claim 9, wherein the bulge is an asymmetric bulge.

11. The engineered guide RNA of claim 9, wherein the bulge is a symmetric bulge.

12. The engineered guide RNA of any one of claims 9-11, wherein the bulge comprises from 1 to 4 nucleotides of the engineered guide RNA and from 0 to 4 nucleotides of the target RNA.

13. The engineered guide RNA of any one of claims 9-11, wherein the bulge comprises from 0 to 4 nucleotides of the engineered guide RNA and from 1 to 4 nucleotides of the target RNA.

14. The engineered guide RNA of claim 10, wherein the asymmetric bulge is an X₁/X₂ asymmetric bulge, wherein X₁ is the number of nucleotides of the target RNA in the asymmetric bulge and X₂ is the number of nucleotides of the engineered guide RNA in the asymmetric bulge, wherein the X₁/X₂ asymmetric bulge is a 0/1 asymmetric bulge, a 1/0 asymmetric bulge, a 0/2 asymmetric bulge, a 2/0 asymmetric bulge, a 0/3 asymmetric bulge, a 3/0 asymmetric bulge, a 0/4 asymmetric bulge, a 4/0 asymmetric bulge, a 1/2 asymmetric bulge, a 2/1 asymmetric bulge, a 1/3 asymmetric bulge, a 3/1 asymmetric bulge, a 1/4 asymmetric bulge, a 4/1 asymmetric bulge, a 2/3 asymmetric bulge, a 3/2 asymmetric bulge, a 2/4 asymmetric bulge, a 4/2 asymmetric bulge, a 3/4 asymmetric bulge, or a 4/3 asymmetric bulge.

15. The engineered guide RNA of claim 11, wherein the symmetric bulge is an X₁/X₂ symmetric bulge, wherein X₁ is the number of nucleotides of the target RNA in the symmetric bulge and X₂ is the number of nucleotides of the engineered guide RNA in the symmetric bulge, and wherein the X₁/X₂ symmetric bulge a 2/2 symmetric bulge, a 3/3 symmetric bulge, or a 4/4 symmetric bulge.

16. The engineered guide RNA of any one of claims 1-8, wherein the structural feature comprises an internal loop.

17. The engineered guide RNA of claim 16, wherein the internal loop comprises an asymmetric internal loop.

18. The engineered guide RNA of claim 16, wherein the internal loop comprises a symmetric internal loop.

19. The engineered guide RNA of claim 17, wherein the asymmetric internal loop is an X₁/X₂ asymmetric internal loop, wherein X₁ is the number of nucleotides of the target RNA in the asymmetric internal loop and X₂ is the number of nucleotides of the engineered guide RNA in the asymmetric internal loop, and wherein the X₁/X₂ asymmetric internal loop is a 5/6 asymmetric internal loop, a 6/5 asymmetric internal loop, a 5/7 asymmetric internal loop, a 7/5 asymmetric internal loop, a 5/8 asymmetric internal loop, a 8/5 asymmetric internal loop, a 5/9 asymmetric internal loop, a 9/5 asymmetric internal loop, a 5/10 asymmetric internal loop, a 10/5 asymmetric internal loop, a 6/7 asymmetric internal loop, a 7/6 asymmetric internal loop, a 6/8 asymmetric internal loop, a 8/6 asymmetric internal loop, a 6/9 asymmetric internal loop, a 9/6 asymmetric internal loop, a 6/10 asymmetric internal loop, a 10/6 asymmetric internal loop, a 7/8 asymmetric internal loop, a 8/7 asymmetric internal loop, a 7/9 asymmetric internal loop, a 9/7 asymmetric internal loop, a 7/10 asymmetric internal loop, a 10/7 asymmetric internal loop, a 8/9 asymmetric internal loop, a 9/8 asymmetric internal loop, a 8/10 asymmetric internal loop, a 10/8 asymmetric internal loop, or a 9/10 asymmetric internal loop, or a 10/9 asymmetric internal loop.

20. The engineered guide RNA of claim 18, wherein the symmetric internal loop is an X₁/X₂ symmetric internal loop, wherein X₁ is the number of nucleotides of the target RNA in the symmetric internal loop and X₂ is the number of nucleotides of the engineered guide RNA in the symmetric internal loop, and wherein the X₁/X₂ symmetric internal loop is a 5/5 symmetric internal loop, a 6/6 symmetric internal loop, a 7/7 symmetric internal loop, a 8/8 symmetric internal loop, a 9/9 symmetric internal loop, a 10/10 symmetric internal loop, a 12/12 symmetric internal loop, a 15/15 symmetric internal loop, or a 20/20 symmetric internal loop.

21. The engineered guide RNA of any one of claims 16-20, wherein the internal loop is formed by at least 5 nucleotides on either the engineered guide RNA or the target RNA.

22. The engineered guide RNA of any one of claims 16-21, wherein the internal loop is formed by from 5 to 1000 nucleotides of either the engineered guide RNA or the target RNA.

23. The engineered guide RNA of any one of claims 16-22, wherein the internal loop is formed by from 5 to 50 nucleotides of either the engineered guide RNA or the target RNA.

24. The engineered guide RNA of any one of claims 16-23, wherein the internal loop is formed by from 5 to 20 nucleotides of either the engineered guide RNA or the target RNA.

25. The engineered guide RNA of any one of claims 1-8, wherein the structural feature comprises a hairpin.

26. The engineered guide RNA of claim 25, wherein the hairpin comprises a non-recruitment hairpin.

27. The engineered guide RNA of claim 25 or 26, wherein a loop portion of the hairpin comprises from about 3 to about 15 nucleotides in length.

28. The engineered guide RNA of any one of claims 1-27, wherein the engineered guide RNA further comprises at least two additional structural features that comprise at least two mismatches.

29. The engineered guide RNA of claim 28, wherein at least one of the at least two mismatches is a G/G mismatch.

30. The engineered guide RNA of any one of claims 1-29, wherein the engineered guide RNA further comprises an additional structural feature that comprises a wobble base pair.

31. The engineered guide RNA of claim 30, wherein the wobble base pair comprises a guanine paired with a uracil.

32. The engineered guide RNA of claim 6-31, wherein the target RNA comprises a 5′ guanosine adjacent to the adenosine in the target RNA that is chemically modified to an inosine by the RNA editing entity.

33. The engineered guide RNA of claim 32, wherein the engineered guide RNA comprises a 5′ guanosine adjacent to the cytosine of the A/C mismatch.

34. The engineered guide RNA of any one of claims 5-33, wherein the RNA editing entity is:
(a) an adenosine deaminase acting on RNA (ADAR);
(b) a catalytically active fragment of (a);
(c) a fusion polypeptide comprising (a) or (b); or
(d) any combination of these.

35. The engineered guide RNA of any one of claims 5-34, wherein the RNA editing entity is endogenous to a cell.

36. The engineered guide RNA of any one of claims 5-35, wherein the RNA editing entity comprises an ADAR.

37. The engineered guide RNA of claim 36, wherein the ADAR comprises human ADAR (hADAR).

38. The engineered guide RNA of claim 36, wherein the ADAR comprises ADAR1, ADAR2, ADAR3, or any combination thereof.

39. The engineered guide RNA of claim 36, wherein the ADAR1 comprises ADAR1p110, ADAR1p150, or a combination thereof.

40. The engineered guide RNA of any one of claims 1-39, wherein the engineered guide RNA comprises a modified RNA base, an unmodified RNA base, or a combination thereof.

41. The engineered guide RNA of any one of claims 1-40, wherein the target RNA is an mRNA molecule.

42. The engineered guide RNA of any one of claims 1-40, wherein the target RNA is a pre-mRNA molecule.

43. The engineered guide RNA of any one of claims 1-42, wherein the target RNA is APP, ABCA4, SERPINA1, HEXA, LRRK2, CFTR, SNCA, MAPT, or LIPA, a fragment any of these, or any combination thereof.

44. The engineered guide RNA of any one of claims 1-42, wherein the target RNA encodes amyloid precursor polypeptide, ATP-binding cassette, sub-family A, member 4 (ABCA4) polypeptide, alpha-1 antitrypsin (AAT) polypeptide, hexosaminidase A enzyme, leucine-rich repeat kinase 2 (LRRK2) polypeptide, CFTR polypeptide, alpha synuclein polypeptide, Tau polypeptide, or lysosomal acid lipase polypeptide.

45. The engineered guide RNA of claim 43 or 44, wherein the target RNA encodes ABCA4 polypeptide.

46. The engineered guide RNA of claim 45, wherein the target RNA comprises a G to A substitution at position 5882, 6320, or 5714, relative to a wildtype ABCA4 gene sequence of accession number NC_000001.11:c94121149-93992837.

47. The engineered guide RNA of claim 45 or 46, wherein the guide-target RNA scaffold comprises one or more structural features selected from TABLE 7, TABLE, 9, TABLE 10, TABLE 11, TABLE 18, or TABLE 19.

48. The engineered guide RNA of any one of claims 45-47, wherein the guide-target RNA scaffold comprises a structural features selected from the group consisting of: (i) one or more X₁/X₂ bulges, wherein X₁ is the number of nucleotides of the target RNA in the bulge and X₂ is the number of nucleotides of the engineered guide RNA in the bulge, and wherein the one or more bulges is a 2/1 asymmetric bulge, a 1/0 asymmetric bulge, a 2/2 symmetric bulge, a 3/3 symmetric bulge, or a 4/4 symmetric bulge; (ii) an X₁/X₂ internal loop, wherein X₁ is the number of nucleotides of the target RNA in the internal loop and X₂ is the number of nucleotides of the engineered guide RNA in the internal loop, and wherein the internal loop is a 5/5 symmetric loop (iii) one or more mismatches, wherein the one or more mismatches is a G/G mismatch, an A/C mismatch, or a G/A mismatch, (iv) a G/U wobble base pair or a U/G wobble base pair, and (v) any combination thereof.

49. The engineered guide RNA of claim 48, wherein the guide-target RNA scaffold comprises a 2/1 asymmetric bulge, a 1/0 asymmetric bulge, a G/G mismatch, an A/C mismatch, and a 3/3 symmetric bulge.

50. The engineered guide RNA of any one of claims 45-49, wherein the engineered guide RNA has a length of from 80 to 175 nucleotides.

51. The engineered guide RNA of any one of claims 45-50, wherein the engineered guide RNA comprises a polynucleotide having at least 80%, at least 85%, at least 90%, at least 95%, at least 97%, at least 99%, or 100% sequence identity to SEQ ID NO: 21, SEQ ID NO: 29, SEQ ID NO: 11, SEQ ID NO: 22, SEQ ID NO: 30, SEQ ID NO: 12, SEQ ID NO: 339-SEQ ID NO: 341, or SEQ ID NO: 292-SEQ ID NO: 296.

52. The engineered guide RNA of claim 45-50, wherein the engineered guide RNA comprises a polynucleotide at least 80%, at least 85%, at least 90%, at least 95%, at least 97%, at least 99%, or 100% sequence identity to any one of SEQ ID NO: 11-34, 58, 218-289, 291-296, or 328-343.

53. The engineered guide RNA of claim 43 or 44, wherein the target RNA encodes LRRK2 polypeptide.

54. The engineered guide RNA of claim 53, wherein the LRRK2 polypeptide comprises a mutation selected from the group consisting of: E10L, A30P, S52F, E46K, A53T, L119P, A211V, C228S, E334K, N363S, V366M, A419V, R506Q, N544E, N551K, A716V, M712V, 1723V, P755L, R793M, 1810V, K871E, Q923H, Q930R, R1067Q, S1096C, Q1111H, I1122V, A1151T, L1165P, I1192V, H1216R, 51228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, 51647T, Y1699C, R1728H, R1728L, L1795F, M1869V, M1869T, L1870F, E1874X, R1941H, Y2006H, 12012T, G2019S, I2020T, T2031S, N2081D, T2141M, R2143H, Y2189C, T2356I, G2385R, V2390M, E2395K, M2397T, L2466H, or Q2490NfsX3.

55. The engineered guide RNA of claim 53 or 54, wherein the guide-target RNA scaffold comprises one or more structural features selected from TABLE 12, TABLE 15, TABLE 25, TABLE 26, TABLE 27, TABLE 17, or TABLE 20.

56. The engineered guide RNA of any one of claims 53-55, wherein the guide-target RNA scaffold comprises one or more structural features selected from the group consisting of: (i) one or more X₁/X₂ bulges, wherein X₁ is the number of nucleotides of the target RNA in the bulge and X₂ is the number of nucleotides of the engineered guide RNA in the bulge, and wherein the one or more bulges is a 0/1 asymmetric bulge, a 2/2 symmetric bulge, a 3/3 symmetric bulge, or a 4/4 symmetric bulge; (ii) one or more X₁/X₂ internal loops, wherein X₁ is the number of nucleotides of the target RNA in the internal loop and X₂ is the number of nucleotides of the engineered guide RNA in the internal loop, and wherein the one or more internal loops is a 5/0 asymmetric internal loop, a 5/4 asymmetric internal loop, a 5/5 symmetric internal loop, a 6/6 symmetric internal loop, a 7/7 symmetric internal loop, or a 10/10 symmetric internal loop; (iii) one or more mismatches, wherein the one or more mismatches is an A/C mismatch, an A/G mismatch, a C/U mismatch, a G/A mismatch, or a C/C mismatch, (iv) a G/U wobble base pair or a U/G wobble base pair, and (v) any combination thereof.

57. The engineered guide RNA of claim 56, wherein the guide-target RNA scaffold comprises a 6/6 symmetrical internal loop, an A/C mismatch, an A/G mismatch, and a C/U mismatch.

58. The engineered guide RNA of any one of claims 53-57, wherein the engineered guide RNA has a length of from 80 to 175 nucleotides.

59. The engineered guide RNA of any one of claims 53-58, wherein the engineered guide RNA comprises a polynucleotide having at least 80%, at least 85%, at least 90%, at least 95%, at least 97%, at least 99%, or 100% sequence identity to SEQ ID NO: 30, SEQ ID NO: 344, or SEQ ID NO: 345.

60. The engineered guide RNA of claims 53-58, wherein the engineered guide RNA comprises a polynucleotide having at least 80%, at least 85%, at least 90%, at least 95%, at least 97%, at least 99%, or 100% sequence identity to any one of SEQ ID NO: 35-42, 46-52, 111-207, or 344-345.

61. The engineered guide RNA of claim 43 or 44, wherein the target RNA encodes SNCA polypeptide.

62. The engineered guide RNA of claim 61, wherein the engineered guide RNA hybridizes to a sequence of the target RNA selected from the group consisting of: a 5′ untranslated region (UTR), a 3′ UTR, and a translation initiation site of an SNCA gene.

63. The engineered guide RNA of claim 61 or 62, wherein the guide-target RNA scaffold comprises one or more structural features selected from TABLE 21, TABLE 23, or TABLE 28.

64. The engineered guide RNA of any one of claims 61-63, wherein the guide-target RNA scaffold comprises one or more structural features selected from the group consisting of: (i) an X₁/X₂ bulge, wherein X₁ is the number of nucleotides of the target RNA in the bulge and X₂ is the number of nucleotides of the engineered guide RNA in the bulge, and wherein the bulge is a 4/4 symmetric bulge; (ii) one or more X₁/X₂ internal loops, wherein X₁ is the number of nucleotides of the target RNA in the internal loop and X₂ is the number of nucleotides of the engineered guide RNA in the internal loop, and wherein the one or more internal loop is a 5/5 symmetric loop, an 8/8 symmetric loop, or a 49/4 asymmetric loop; (iii) one or more mismatches, wherein the one or more mismatches is an A/C mismatch, a G/G mismatch, a G/A mismatch, a U/C mismatch, or an A/A mismatch, (iv) any combination thereof.

65. The engineered guide RNA of claim 64, wherein the engineered guide RNA has a length of from 80 to 175 nucleotides.

66. The engineered guide RNA of any one of claims 61-64, wherein the engineered guide RNA comprises a polynucleotide having at least 80%, at least 85%, at least 90%, at least 95%, at least 97%, at least 99%, or 100% sequence identity to any one of SEQ ID NO: 59-101, 104-108, and 208-217.

67. The engineered guide RNA of claim 43 or 44, wherein the target RNA encodes SERPINA1.

68. The engineered guide RNA of claim 67, wherein the target RNA comprises a G to A substitution at position 9989, relative to a wildtype SERPINA1 gene sequence of accession number NC_000014.9:c94390654-94376747.

69. The engineered guide RNA of claim 67 or 68, wherein the guide-target RNA scaffold comprises one or more structural features selected from TABLE 5, TABLE 29, TABLE 30, TABLE 31, TABLE 32, TABLE 33, TABLE 34, TABLE 35, or TABLE 36.

70. The engineered guide RNA of any one of claims 67-69, wherein the guide-target RNA scaffold comprises one or more structural features selected from the group consisting of: (i) one or more X₁/X₂ bulges, wherein X₁ is the number of nucleotides of the target RNA in the bulge and X₂ is the number of nucleotides of the engineered guide RNA in the bulge, and wherein the bulge is a 0/2 asymmetric bulge, a 0/3 asymmetric bulge, a 1/0 asymmetric bulge, a 2/0 asymmetric bulge, a 2/2 symmetric bulge, a 3/0 asymmetric bulge, a 2/2 symmetric bulge, or a 3/3 symmetric bulge; (ii) an X₁/X₂ internal loop, wherein X₁ is the number of nucleotides of the target RNA in the internal loop and X₂ is the number of nucleotides of the engineered guide RNA in the internal loop, and wherein the internal loop is a 5/5 symmetric internal loop; (iii) one or more mismatches, wherein the one or more mismatches is an A/C mismatch, an A/A mismatch, and a G/A mismatch, (iv) a G/U wobble base pair, or a U/G wobble base pair; and (v) any combination thereof.

71. The engineered guide RNA of claim 70, wherein the engineered guide RNA has a length of from 80 to 175 nucleotides.

72. The engineered guide RNA of any one of claims 67-71, wherein the engineered guide RNA comprises a polynucleotide having at least 80%, at least 85%, at least 90%, at least 95%, at least 97%, at least 99%, or 100% sequence identity to any one of SEQ ID NO: 6-10, 102-103 or 297-327.

73. The engineered guide RNA of claim 1-72, wherein the base of the nucleotide of the target RNA that is modified by the RNA editing entity is comprised in a point mutation of the target RNA.

74. The engineered guide RNA of claim 73, wherein the point mutation comprises a missense mutation.

75. The engineered guide RNA of claim 73, wherein the point mutation is a nonsense mutation.

76. The engineered guide RNA of claim 75, wherein the nonsense mutation is a premature UAA stop codon.

77. The engineered guide RNA of any one of claims 1-76, wherein the structural feature increases selectivity of editing a target adenosine in the target RNA relative to an otherwise comparable guide RNA lacking the structural feature.

78. The engineered guide RNA of any one of claims 1-77, wherein the structural feature decreases an amount of RNA editing of local off-target adenosines within 200, within 100, within 50, within 25, within 10, within 5, within 2, or 1 within 1 nucleotide 5′ or 3′ of a target adenosine in the target RNA by the RNA editing entity, relative to an otherwise comparable guide RNA lacking the structural feature.

79. An engineered RNA comprising:
(a) the engineered guide RNA of any one of claims 1-78,
(b) a U7 snRNA hairpin sequence, a SmOPT sequence, or a combination thereof.

80. The engineered RNA of claim 79, wherein the U7 hairpin has a sequence of
(SEQ ID NO: 389)
TAGGCTTTCTGGCTTTTTACCGGAAAGCCCCT
or
(SEQ ID NO: 394)
CAGGTTTTCTGACTTCGGTCGGAAAACCCCT.

81. The engineered RNA of claim 79, wherein the SmOPT sequence has a sequence of
(SEQ ID NO: 390)
AATTTTTGGAG.

82. A polynucleotide encoding the engineered guide RNA of any one of claims 1-78 or the engineered RNA of any one of claims 79-81.

83. A delivery vector comprising the engineered guide RNA of any one of claims 1-78, the engineered RNA of any one of claims 79-81, or the polynucleotide of claim 82.

84. The delivery vector of claim 83, wherein the delivery vector is a viral vector.

85. The delivery vector of claim 84, wherein the viral vector is an adeno-associated viral (AAV) vector or a derivative thereof.

86. The delivery vector of claim 85, wherein the AAV vector is from an adeno-associated virus having a serotype selected from AAV1, AAV2, AAV3, AAV4, AAV5, AAV6, AAV7, AAV8, AAV9, AAV10, AAV 11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68.

87. The delivery vector of claim 85 or 86, wherein the AAV vector is a recombinant AAV (rAAV) vector, a hybrid AAV vector, a chimeric AAV vector, a self-complementary AAV (scAAV) vector, a single-stranded AAV or any combination thereof.

88. The delivery vector of any one of claims 85-87, wherein the AAV vector comprises a genome comprising a replication gene and inverted terminal repeats from a first AAV serotype and a capsid protein from a second AAV serotype.

89. The delivery vector of any one of claims 85-88, wherein the AAV vector is an AAV 2/5 vector, an AAV 2/6 vector, an AAV 2/7 vector, an AAV2/8 vector, or an AAV 2/9 vector.

90. The delivery vector of claim 88, wherein the inverted terminal repeats comprise a 5′ inverted terminal repeat, a 3′ inverted terminal repeat, and a mutated inverted terminal repeat.

91. The delivery vector of claim 90, wherein the mutated inverted terminal repeat lacks a terminal resolution site.

92. A pharmaceutical composition comprising:
(a) engineered guide RNA of any one of claims 1-78, the engineered RNA of any one of claims 79-81, the polynucleotide of claim 82, or the delivery vector of any one of claims 83-91, and
(b) a pharmaceutically acceptable: excipient, carrier, or diluent.

93. The pharmaceutical composition of claim 92, in unit dose form.

94. The pharmaceutical composition of claim 92 or 93, further comprising an additional therapeutic agent.

95. The pharmaceutical composition of claim 94, wherein the additional therapeutic agent comprises an ammonia reducer, a beta blocker, a synthetic hormone, an antibiotic, or an antiviral drug, a vascular endothelial growth factor (VEGF) inhibitor, a stem cell treatment, a vitamin or modified form thereof, or any combination thereof.

96. A method of editing a target RNA in a cell, the method comprising: administering to the cell an effective amount of the engineered guide RNA of any one of claims 1-78, the engineered RNA of any one of claims 79-81, the polynucleotide of claim 82, the delivery vector of any one of claims 83-91, or the pharmaceutical composition of any one of claims 92-95.

97. A method of treating a disease in a subject, the method comprising administering to the subject an effective amount of the engineered guide RNA of any one of claims 1-78, the engineered RNA of any one of claims 79-81, the polynucleotide of claim 82, the delivery vector of any one of claims 83-91, or the pharmaceutical composition of any one of claims 92-95.

98. The method of claim 96, wherein the engineered guide RNA is administered as a unit dose.

99. The method of claim 98, wherein the unit dose is an amount sufficient to treat the subject.

100. The method of any one of claims 96-99, wherein the administering is intrathecal, intraocular, intravitreal, retinal, intravenous, intramuscular, intraventricular, intracerebral, intracerebellar, intracerebroventricular, intraperenchymal, subcutaneous, or a combination thereof.

101. The method of any one of claims 96-100, wherein the disease comprises a neurological disease.

102. The method of claim 101, wherein the neurological disease comprises Parkinson's disease, Alzheimer's disease, a Tauopathy, or dementia.

103. The method of claim 101 or 102, wherein the neurological disease is associated with elevated levels of SNCA polypeptide, relative to a healthy subject that does not have the neurological disease or condition.

104. The method of claim 103, wherein the engineered guide RNA hybridizes to a sequence of a target RNA encoding the SNCA polypeptide selected from the group consisting of: a 5′ untranslated region (UTR), a 3′ UTR, and a translation initiation site of SNCA; wherein hybridization produces a guide-target RNA scaffold that is a substrate for an RNA editing entity that chemically modifies a base of a nucleotide in the sequence of the target RNA, thereby reducing levels of the SNCA polypeptide.

105. The method of claim 104, wherein the engineered guide RNA hybridizes to a sequence of a target RNA encoding the translation initiation site of SNCA.

106. The method of any one of claims 103-105, wherein the engineered guide RNA comprises a polynucleotide having at least 80%, at least 85%, at least 90%, at least 95%, at least 97%, at least 99%, or 100% sequence identity to any one of SEQ ID NO: 59-101, 104-108, and 208-217.

107. The method of any one of claims 103-106, wherein the engineered guide RNA comprises has a percent on-target editing for ADAR2 of at least about 90%.

108. The method of claim 101 or 102, wherein the neurological disease is associated with a mutation of an LRRK2 polypeptide encoded by the target RNA, wherein the mutation is selected from the group consisting of: E10L, A30P, S52F, E46K, A53T, L119P, A211V, C228S, E334K, N363S, V366M, A419V, R506Q, N544E, N551K, A716V, M712V, 1723V, P755L, R793M, 1810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, 12012T, G2019S, I2020T, T2031S, N2081D, T2141M, R2143H, Y2189C, T2356I, G2385R, V2390M, E2395K, M2397T, L2466H, or Q2490NfsX3.

109. The method of claim 101 or 102, wherein the neurological disease is associated with a mutation of an LRRK2 polypeptide encoded by the target RNA, wherein the mutation is a G2019S mutation.

110. The method of any one of claims 108-114, wherein the engineered guide RNA comprises a polynucleotide having at least 80%, at least 85%, at least 90%, at least 95%, at least 97%, at least 99%, or 100% sequence identity to any one of SEQ ID NO: 35-42, 46-52, 111-207, or 344-345.

111. The method of any one of claims 108-110, wherein the engineered guide RNA comprises has a percent on-target editing for ADAR1 of at least about 60% or a percent on-target editing for ADAR2 of at least about 90%.

112. The method of any one of claims 96-100, wherein the disease comprises a liver disease.

113. The method of claim 112, wherein the liver disease comprises liver cirrhosis.

114. The method of claim 112, wherein the liver disease is alpha-1 antitrypsin (AAT) deficiency.

115. The method of claim 114, wherein the AAT deficiency is associated with a G to A substitution at position 9989 of a wildtype SERPINA1 gene sequence of accession number NC_000014.9:c94390654-94376747.

116. The method of claim 114 or 115, wherein the engineered latent wherein the engineered guide RNA comprises a polynucleotide having at least 80%, at least 85%, at least 90%, at least 95%, at least 97%, at least 99%, or 100% sequence identity to any one of SEQ ID NO: 6-10, 102-103 or 297-327.

117. The method of any one of claims 114-116, wherein the engineered guide RNA comprises has a percent on-target editing for ADAR1 of at least about 60% or a percent on-target editing for ADAR2 of at least about 90%.

118. The method of any one of claims 96-100, wherein the disease is a macular degeneration.

119. The method of claim 118, wherein the macular degeneration is Stargardt Disease.

120. The method of claim 119, wherein the Stargardt disease is associated with a G to A substitution at position 5882, 6320, or 5714 of a wildtype ABCA4 gene sequence of accession number NC_000001.11:c94121149-93992837.

121. The method of claim 120, wherein the Stargardt disease is associated with a G to A substitution at position 5882.

122. The method of claim 119 or 120, wherein the engineered guide RNA comprises a polynucleotide having at least 80%, at least 85%, at least 90%, at least 95%, at least 97%, at least 99%, or 100% sequence identity to any one of SEQ ID NO: 11-34, 58, 218-289, 291-296, or 328-343.

123. The method of any one of claims 120-122, wherein the engineered guide RNA comprises has a percent on-target editing for ADAR1 of at least about 70% or a percent on-target editing for ADAR2 of at least about 80%.

124. The method of any one of claims 96-123, wherein the subject is diagnosed with the disease or the condition.

125. The engineered guide RNA of any one of claims 1-78, the engineered RNA of any one of claims 79-81, the polynucleotide of claim 82, the delivery vector of any one of claims 83-91, or the pharmaceutical composition of any one of claims 92-95, for use as a medicament.

126. The engineered guide RNA of any one of claims 1-78, the engineered RNA of any one of claims 79-81, the polynucleotide of claim 82, the delivery vector of any one of claims 83-91, or the pharmaceutical composition of any one of claims 92-95, for use in treatment of a neurological disease.

127. The engineered guide RNA, polynucleotide, delivery vector, or pharmaceutical composition for the use of claim 127, wherein the neurological disease is Parkinson's disease, Alzheimer's disease, a Tauopathy, or dementia.

128. The engineered guide RNA of any one of claims 1-78, the engineered RNA of any one of claims 79-81, the polynucleotide of claim 82, the delivery vector of any one of claims 83-91, or the pharmaceutical composition of any one of claims 92-95, for use in treatment of a liver disease.

129. The engineered guide RNA, polynucleotide, delivery vector, or pharmaceutical composition for the use of claim 128, wherein the liver disease comprises liver cirrhosis.

130. The engineered guide RNA, polynucleotide, delivery vector, or pharmaceutical composition for the use of claim 128, wherein the liver disease is alpha-1 antitrypsin (AAT) deficiency.

131. The engineered guide RNA of any one of claims 1-78, the engineered RNA of any one of claims 79-81, the polynucleotide of claim 82, the delivery vector of any one of claims 83-91, or the pharmaceutical composition of any one of claims 92-95, for use in treatment of macular degeneration.

132. The engineered guide RNA, polynucleotide, delivery vector, or pharmaceutical composition for the use of claim 131, wherein the macular degeneration is Stargardt disease.

133. Use of the engineered guide RNA of any one of claims 1-78, the engineered RNA of any one of claims 79-81, the polynucleotide of claim 82, the delivery vector of any one of claims 83-91, or the pharmaceutical composition of any one of claims 92-95, for the manufacture of a medicament.

134. Use of the engineered guide RNA of any one of claims 1-78, the engineered RNA of any one of claims 79-81, the polynucleotide of claim 82, the delivery vector of any one of claims 83-91, or the pharmaceutical composition of any one of claims 92-95, for the manufacture of a medicament for the treatment of a neurological disease, a liver disease or macular degeneration.
